(19) United States
(12) Patent Application Publication
(10) Pub. No.: US 2021/0024997 A1
(43) Pub. Date: Jan. 28, 2021

(54) CELL ATLAS OF HEALTHY AND DISEASED TISSUES

(71) Applicants: Massachusetts Institute of Technology, Cambridge, MA (US); Seattle Children's Hospital DBA Seattle Children's Research Institute, Seattle, WA (US)

(72) Inventors: Alexander K. Shalek, Cambridge, MA (US); Carly Ziegler, Cambridge, MA (US); Shaina Carroll, Cambridge, MA (US); Leslie Kean, Seattle, WA (US); Lucrezia Colonna, Seattle, WA (US)

(21) Appl. No.: 16/756,625

(22) PCT Filed: Oct. 16, 2018
(86) PCT No.: PCT/US2018/056166

§ 371 Date: Apr. 16, 2020

Related U.S. Application Data
(60) Provisional application No. 62/573,015, filed on Oct. 16, 2017.

(51) Int. Cl. C12Q 1/6876; C12Q 1/6881

(52) U.S. Cl. CPC C12Q 1/6876; C12Q 2600/158; C12Q 1/6881

(57) ABSTRACT

Embodiments disclosed herein provide a pan-tissue cell atlas of healthy and diseased subjects obtained by single cell sequencing. The present invention discloses novel markers for cell types. Moreover, genes associated with disease, including HIV infection and tuberculosis are identified. The invention provides for diagnostic assays based on gene markers and cell composition, as well as therapeutic targets for controlling immune regulations and cell-cell communication of the cell types disclosed herein. In addition, novel cell types and methods of quantitating, detecting and isolating the cell types are disclosed.

### CROSS-REFERENCE TO RELATED APPLICATIONS

This application claims the benefit of U.S. Provisional Application No. 62/573,015, filed Oct. 16, 2017. The entire contents of the above-identified application are hereby fully incorporated herein by reference.

### STATEMENT REGARDING FEDERALLY SPONSORED RESEARCH

This invention was made with government support under Grant No. HL095791 awarded by the National Institutes of Health. The government has certain rights in the invention.

### REFERENCE TO AN ELECTRONIC SEQUENCE LISTING

The contents of the electronic sequence listing (BROD-2830US.ST25.txt”; Size is 12 Kilobytes and it was created on Aug. 6, 2020) is herein incorporated by reference in its entirety.

### TECHNICAL FIELD

The subject matter disclosed herein is generally directed to use of tissue, cellular and gene biomarkers to determine the physiological state of a cell or tissue of interest. The subject matter further relates to a cell atlas of healthy tissues and a matched cell atlas of infectious disease and biomarkers thereof. cell types in healthy and disease states. The subject matter further relates to novel cell specific and disease specific markers. and infectious disease in

This invention relates generally to compositions and methods identifying and exploiting target genes or target gene products that modulate, control or otherwise influence cell-cell communication, differential expression, immune response in a variety of therapeutic and/or diagnostic indications.

### BACKGROUND

Immune systems play an essential role in ensuring our health. From decades of laboratory and clinical work, there has been a basic understanding of immune balance and its importance for a healthy immune system. For example, hyperactivity can lead to allergy, inflammation, tissue damage, autoimmune disease and excessive cellular death. On the other hand, immunodeficiency can lead to outgrowth of cancers and the inability to kill or suppress external invaders. The immune system has evolved multiple modalities and redundancies that balance the system, including but not limited to memory, exhaustion, anergy, and senescence. Despite this basic understanding, a comprehensive landscape of immune regulations remains missing. Given the importance of the immune system, a systematic understanding of immune regulations on cell, tissue, and organism levels is crucial for clinicians and researchers to efficiently diagnose and develop treatments for immune system related disease.

Different cells and tissues in a diseased organism are often not impacted at the same level. Analyzing immune regulations with a comprehensive approach allows for identification of cells and tissues that are impacted and that are representative of the disease, interaction between cells, as well as pathways that can be specifically targeted to restore diseased cell or tissues to a normal state. In practice, certain tissues or specimens, for example blood or body fluids, are more easily obtainable than others from a patient. A systematic understanding of immune responses allows clinicians to use easily obtainable tissues as a proxy to diagnose disease and monitor disease state through easily obtainable tissues, and may further allow for treatment or amelioration of symptoms by restoring the state of suppressed immune cells or eliminating severely infected cells, for example, cells impacted with a chronic infection such as HIV infected cells/MTB infected cells.

Despite years of clinical work, essential information including location and identity of the pathogen hosting cells or tissues, immunologic response and pathways involved in the infection and response status of such disease causing infections remain unclear. A comprehensive understanding focusing on diseased as well as healthy organisms will be able to locates key cells and tissues that represent the disease, location, identity, and phenotype of the disease harboring cells, pathways and mechanisms involved in disease response and pathogen replication, thus help developing diagnosis as well as treatment methods.

Reliable diagnosing of disease states and evaluation of therapies remains problematic. In human subjects, many cell type and tissues are inaccessible to non-invasive methods and further may be difficult to locate and test even where, for example, biopsy procedures are available. Such difficulties extend to non-human animals, including but not limited to non-human primates. For example, animal tissues may be available from animals that cannot be obtained from living human subjects, but such tissues may be inaccessible for other reasons, frequently expense.

### SUMMARY

In certain example embodiments, the present invention provides novel markers for cell types and physiological states of tissues of interests.

In one aspect, the present invention provides for a method of determining a physiological state of a first cell or tissue in a subject, the method comprising: measuring a physiological state of a second cell or tissue in the subject that is correlated with the physiological state of the first cell or tissue, wherein the correlation comprises a correlation between tissue types, cell types, or tissue types and cell types.

In another aspect, the present invention provides for a method of determining the effect of a modulating agent on a first cell or tissue in a subject, the method comprising: measuring the effect of the modulating agent on a second cell or tissue in the subject, wherein the physiological state of the second cell or tissue is correlated with the effect of the modulating agent on the first cell or tissue, wherein the correlation comprises a correlation between tissue types, cell types, or tissue types and cell types.

In certain embodiments, the composition and/or quantity of cell types in different tissues is correlated, or the same cell types in different tissues are correlated, or different cell types are correlated. In certain embodiments, the second cell or tissue is correlated with the first cell or tissue in another organism, whereby the correlation is used as a proxy to determine the physiological state of the first cell or tissue in the subject.

In certain embodiments, the organism is a non-human primate. In certain embodiments, the non-human primate is a Rhesus macaque.

In certain embodiments, the correlation is determined by measuring gene expression profiles in two or more cells or tissues obtained from the organism. In certain embodiments, the correlated physiological states of the first and second cells or tissues are the same physiological states. In certain embodiments, the correlated physiological states of the first and second cells or tissues are different physiological states. In certain embodiments, the physiological state of the second cell or tissue is measured by a gene expression profile comprising one or more genes. In certain embodiments, the physiological state of the second cell or tissue is measured by a gene expression profile comprising one or more gene clusters. In certain embodiments, the gene expression profile comprises single cell expression profiles. In certain embodiments, the gene clusters comprise one or more principle component genes. In certain embodiments, the one or more gene clusters comprise genes having similar function. In certain embodiments, the one or more gene clusters comprise genes that are co-regulated. In certain embodiments, the genes are co-regulated in the tissue or cell during disease. In certain embodiments, the one or more gene clusters comprise genes of a pathway.

In certain embodiments, the cell type is an immune cell or the tissue type is an immune tissue type. In certain embodiments, the cells comprise T cells from mesenteric lymph node, inguinal lymph node, CNS, jejunun, spleen, tonsil, or bone marrow. In certain embodiments, the cells comprise macrophages. In certain embodiments, the cells comprise pneumocytes or NK cells. In certain embodiments, the cells comprise cells of axillary lymph node, colon, ileum, liver, spleen, or thymus. In certain embodiments, the cell or tissue type is a diseased cell or tissue type.

In certain embodiments, the modulating agent is an immune modulating agent.

In certain embodiments, the physiological state comprises a disease state or an immunological state. In certain embodiments, the physiologic state indicates resistance or sensitivity to a therapy.

In certain embodiments, the second cell is a circulating immune cell and the physiological state is an immune state in a tissue.

In another aspect, the present invention provides for a method of identifying a biomarker as a proxy for a physiological state of a cell or tissue, the method comprising determining the expression profile of one or more genes in a test cell or tissue obtained from an organism, and identifying the expression profile in the test cell or tissue as a proxy for the physiological state of a second cell or tissue if the expression profile in the test cell or tissue is correlated with the expression profile in the second cell or tissue obtained from the organism.

In another aspect, the present invention provides for a method of identifying a biomarker as a proxy for a physiological state of a cell or tissue, the method comprising determining an expression profile of one or more genes in a test cell or tissue obtained from an organism that correlates with the expression profile in a second cell or tissue obtained from the organism.

In certain embodiments, the expression profile comprises one or more single cell expression profiles and the single cell expression profiles in the test cell or tissue correlates to the single cell expression profiles in the second cell or tissue. In certain embodiments, the test cell or tissue is from the same species as the second cell or tissue. In certain embodiments, the test cell or tissue and the second cell or tissue are from a non-human primate. In certain embodiments, the test cell or tissue and the second cell or tissue are from a Rhesus macaque. In certain embodiments, the expression profile determined in the test cell or tissue is a proxy for the physiological state of the second cell in a different species, preferably a related species. In certain embodiments, the test cell or tissue and the second cell or tissue are from different non-human primates. In certain embodiments, the test cell or tissue is from a human and the second cell or tissue is from a non-human primate. In certain embodiments, the biomarker identified in the non-human primate is used to determine the physiological state of a second cell or tissue in a human subject by detection or measuring the biomarker in the first cell or tissue in the human subject. In certain embodiments, the physiological state comprises a disease state or an immunological state. In certain embodiments, the physiologic state indicates resistance or sensitivity to a therapy.

In another aspect, the present invention provides for a method of diagnosing the physiological state of a cell or tissue in a subject, the method comprising measuring the expression of a biomarker in a test cell or tissue of the subject, wherein the biomarker was identified as a proxy for the physiological state of the diagnosed cell or tissue by determining the expression profile of the biomarker in a first cell or tissue, and identifying the expression profile in the first cell or tissue as a proxy for the physiological state of a second cell or tissue if the expression profile in the first cell or tissue is correlated with the expression profile in the second cell or tissue.

In certain embodiments, the first cell or tissue is from the same species as the second cell or tissue. In certain embodiments, the first cell or tissue and the second cell or tissue are from a non-human primate. In certain embodiments, the first cell or tissue and the second cell or tissue are from a Rhesus macaque.

In another aspect, the present invention provides for a method of identifying a biomarker as a proxy for determining the effect of a modulating agent on a cell or tissue in a subject, the method comprising determining an expression profile of one or more genes in a test cell or tissue obtained from an organism treated with the modulating agent that correlates with the expression profile in a second cell or tissue obtained from the treated organism.

In another aspect, the present invention provides for a method of identifying cell interactions comprising: providing single cell gene expression profiles obtained from sequencing single cells from one or more tissues from a non-human primate; determining expression of receptor/ligand pairs on the single cells from the one or more tissues; and determining cells that express a receptor and cells that express the ligand for the receptor.

In certain embodiments, cell interactions are determined in a diseased non-human primate.

In another aspect, the present invention provides for a method of identifying biomarkers of tissue homing comprising: generating single cell expression profiles of PBMC's obtained from two or more tissues of a non-human primate; and identifying tissue specific markers expressed by the PBMCs.

In another aspect, the present invention provides for a method of identifying the tissue of origin of PBMCs comprising detecting in PBMCs obtained from a subject one or markers selected from a marker described herein.

In certain embodiments, the tissue of origin of macrophages is identified by detecting in macrophages one or markers selected from one or more groups consisting of: S100A8, HBB, MNP1A, CAMP, LOC710097, gene 24745, gene 18845, LOC703853, LOC706282 and RTD1B; LOC106994075, PLAC8, CLEC9A, GZMB, IRF8, FCER1A, KNG1, IGFBP6, CCDC50 and NCOA7; C1QB, SEPP1, FABP4, C1QC, GPNMB, APOE, ACP5, YMRM176B, ADAMDEC1 and CCDC152; and/or S100A6, FCGR3, VCAN, FGR, LILRB1, FCN1, AHNAK, FN1, C5AR1, TIMP1.

In certain embodiments, the method further comprises using the PBMCs originating from a tissue of interest as a proxy for the physiological state of the tissue of interest.

In certain embodiments, the expression profile in a first tissue is a proxy for the expression profile in a second tissue. In certain embodiments, the expression of one or more genes selected from a marker described herein in the first tissue is a proxy for the physiological state of the second tissue.

In another aspect, the present invention provides for a method of identifying tissues and cells that are reservoirs for HIV comprising determining expression of SHIV genes in tissues and/or single cells obtained from a non-human primate infected with SHIV and treated with antiretroviral therapy. In certain embodiments, SHIV is reactivated in the tissues and/or single cells before determining expression.

In another aspect, the present invention provides for a method of identifying tissues and cells that are reservoirs for HIV comprising determining expression of HIV genes in tissues and/or single cells obtained from a subject infected with HIV and treated with antiretroviral therapy. In certain embodiments, HIV is reactivated in the tissues and/or single cells before determining expression.

In certain embodiments, the tissues and/or single cells are obtained from lymph nodes. In certain embodiments, the diseased cell or tissue type is infected with HIV. In certain embodiments, the physiological state comprises an immunological state associated with HIV infection.

In certain embodiments, the diseased cell or tissue type is infected with MTB. In certain embodiments, the physiological state comprises an immunological state associated with MTB infection.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Balance in the immune system determines health vs. disease. Hyperactivity can lead to tissue damage, allergy, inflammation, and cell death. Immunodeficiency can lead to outgrowth of cancers or external pathogens.

FIG. 2—Host-Pathogen Dynamics of HIV Infection. HIV preferentially infects CD4 T cells, reverse transcribes its DNA, and integrates into the host genome. Infection progresses through a spike in viral load, followed by a progressive decrease in CD4+ T cell count. Because of the high plasma viral load, and because T cells migrate throughout different locations, virtually all tissues can be exposed to the virus, causing profound, and often irreversible changes to the adaptive and innate immune systems, and establishing a permanent pool of integrated HIV termed the “reservoir.”

FIG. 3—Lymph node cells stain positive for HIV proteins such as p24 by flow cytometry indicating a significant fraction of cells are actively producing virus.

FIG. 4—Lymph node from an HIV-infected, antiretroviral-treated patient.

FIG. 5—HIV infection status of single cells. Detection of host mRNA and HIV-1 RNA from the same cell.

FIG. 6—HIV infection status of single cells. Detection of host mRNA and HIV-1 RNA from the same cell.

FIG. 7—Cellular identities of Active HIV Reservoir. Top: Single cell RNA detection distinguishes cells, including markers and pathways, that contribute to ongoing HIV replication. Bottom: Differential expression between HIV⁺ and HIV⁻ cells shown by gag-pol abundance identifies genes that drive HIV replication such as transcription factors that bind to HIV promoter regions. Genes associated with metabolism of anti-retroviral drugs are also detected and novel differentially expressed genes identified.

FIG. 8A-8E—MTB-infected macrophages. FIG. 8A Macrophage transcript mapping by macrophage/MTB ratio. FIG. 8B Examples of pathway expression correlated with MTB MOI. FIG. 8C Cellular response to variable copy number of internalized TB indicated by single cells, individually correlated with MTB/cell. FIG. 8D Spearman correlation between MTB/cell and gene expression. FIG. 8E Correlation between MTB/cell and pathway components at low MOI (top) and high MOI (bottom).

FIG. 9—Genes and pathways associated with TB abundance.

FIG. 10—Expression of macrophage genes and pathways enriched in cells infected with TP singly or as aggregates. FIG. 10A Genes and pathways enriched in cells infected with aggregates (red) or singles (blue). FIG. 10B Differential enrichment of cell death (left) and TNF (right) pathways in cells infected as aggregates or singles.

FIG. 11—Non-human primate model showing examples of cells and tissues useful for elaborating gene signatures associated with diseases and disorders.

FIG. 12—Single cell profiles define cells by tissue (left) and cell type (right).

FIG. 13—Single cell transcriptome expression profiles cluster by cell type.

FIG. 14—CD3E++CD3D++CD3G+ cells by tissue and cell type.

FIG. 15A—Tissue specific behavior of macrophages; FIG. 15B charts number of tissue specific cells of macrophages; FIG. 15C single cell transcriptomes of macrophages identify genes that define them. FIG. 15D single cell transcriptomes of macrophages identify tissue specific sub sets.

FIG. 16—Macrophage expression profiles correspond with tissues of origin.

FIG. 17—Single cell profiles define cells by tissue (left) and cell type (right).

FIG. 18—Identification of pneumocyte (FIG. 18A) and NK (FIG. 18B) cell clusters.

FIG. 19—Gene expression in pneumocytes indicates tissue-dependence.

FIG. 20—Gene expression in NK cells indicates common functions and potential differences driven by tissue-of-origin.

FIG. 21—Cell resolution looking at individual tissues.

FIG. 22—Cell expression profiles by tissue.

FIG. 23—Gene expression in PBMCs showing individual cell types and correlation with gene groups.

FIG. 24—Gene expression of cells in Ileum showing individual cell types and correlation with gene groups.

FIG. 25A-25C—Single cell genomics FIG. 25A Single cell genomics of cells from lymphoid tissue from healthy and SHIV-infected Rhesus macaques defines specific cell subsets. FIG. 25B Certain subsets have equal representation between healthy and SHIV, such as CD8 T cells or macrophages, while CD4 T cells and B cells, show major deviations due to prior SHIV infection. FIG. 25C Differential expression of genes in healthy and SHIV-infected CD4 T cells. As in humans, animals with suppressed viral replication as detected in blood show signatures in lymphoid resident T cells associated with ongoing viral replication and response to virus.

FIG. 26—Comparison of differentially expressed genes between HIV⁺ and HIV⁻ T cells in human lymph nodes with SHIV⁺ and SHIV⁻ T cells in non-human primates shows significant overlap.

FIG. 27A-27D—Impact of chronic SHIV infection on different tissue niches. FIG. 27A Single cell genomics of cells from lymphoid tissue and ileum compared. FIG. 27B In the mesenteric LN, T cells are affected by prior HIV infection, but in the ileum, a significant effect is not observed. FIG. 27C In the small intestine, T cells are more similar, but largest differential expression occurs among the epithelial enterocytes. FIG. 27D Identification of cell subsets altered by SHIV infection.

FIG. 28—Numbers of UMIs detected in 12 tissues obtained from a single healthy Rhesus macaque using shallow sequencing (3 seq-well arrays/NextSeq Run).

FIG. 29—T cell phenotypes across tissue of origin. Shown are bar graphs showing number of T cells detected in each tissue and the percent of tissue. tSNE plot showing T cells sorted by tissues. Cells were gated on CD3, TRBC. and TRAC.

FIG. 30—T cell phenotypes across tissue of origin. tSNE plots showing T cells sorted by tissue and cell type. Cells were gated on CD3, TRBC. and TRAC.

FIG. 31—Identification of markers of recent emigrants/immigrants (e.g., markers for tissue homing and specificity). tSNE plots showing cells sorted by tissue and with PBMCs highlighted.

FIG. 32—Schematic showing identification of cell-cell interactions and calculating an interaction score.

FIG. 33A-33D—FIG. 33A tSNE plot of cell types obtained from the Ileum. FIG. 33B Plot showing cell-cell pairing strength. The pairing uses all transmembrane molecules and does not use secreted interactions. FIG. 33C Circos plots for indicated cell types. Edges=coexpression of Receptorₓ and Ligand_{y}. Weight of edges corresponds to the interaction score. FIG. 33D Differential receptor ligand potential between health and disease.

FIG. 34—Schematic showing tissue workflow for constructing a comprehensive atlas of anti-retroviral therapy (ART) resistant and latent SHIV reservoir.

FIG. 35—Schematic showing tissue workflow for activating/reversing latency in single cells to increase detection of SHIV+ cells.

FIG. 36—Comparison of healthy vs. disease in non-human primates. tSNE plots and heatmap from two healthy macaques and two SHIV infected macaques. T cells were gated using CD3+ and were obtained from the mesenteric lymph node.

FIG. 37—Schematic showing computational methods for determining differential coexpression networks in healthy vs. disease (SHIV).

FIG. 38—Differential coexpression networks in healthy vs. disease (SHIV). Mesenteric lymph node T cells were analyzed.

FIG. 39—Comparison of pathways expressed in mesenteric LN from 2 Healthy Controls vs. 2 SHIV+, ARV-treated animals.

FIG. 40—A healthy cell atlas of lymphoid tissues. tSNE plots from lymphoid tissue obtained from healthy animals highlighted by tissue and cell types.

FIG. 41—Diagram showing computation modules for Transcriptomic Interaction Networks (TINDIR) to discover intercellular relationships.

FIG. 42—Diagram showing computation modules for Transcriptomic Interaction Networks (TINDIR) to discover intercellular relationships.

FIG. 43—Transcriptomic Interaction Networks (TINDIR) data input.

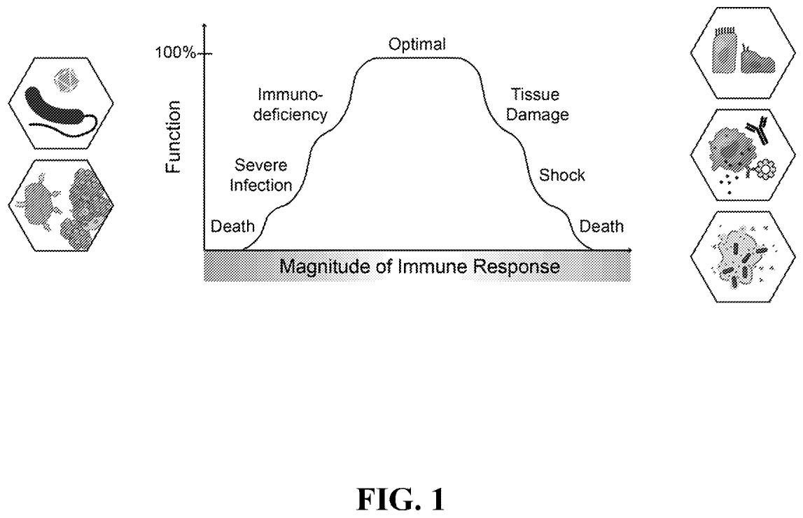

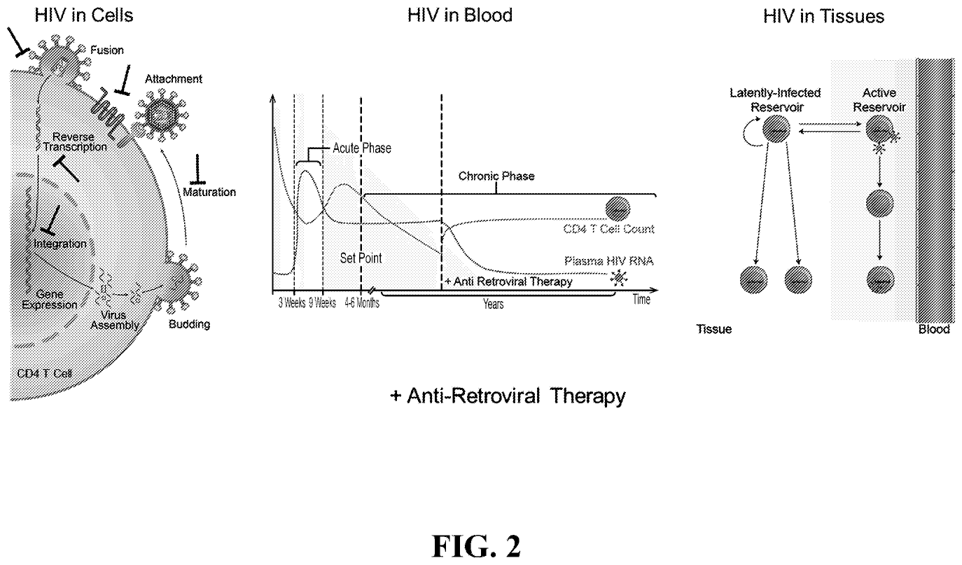

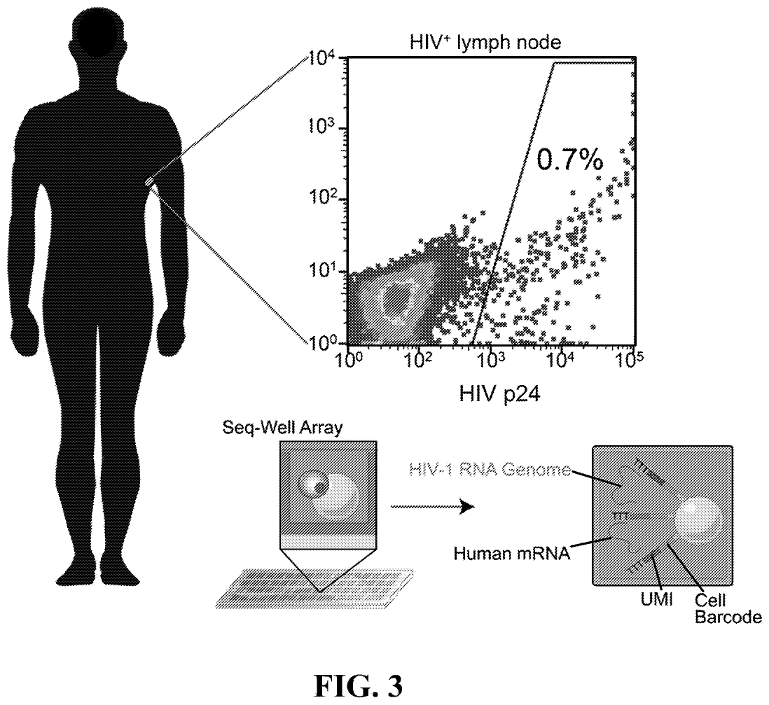

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +1-10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide novel markers for cell types and physiological states of tissues of interests. Moreover, genes associated with chronic infection and disease, including HIV infection and tuberculosis (TB) are identified. The invention provides for diagnostic assays based on gene markers and cell composition, as well as therapeutic targets for controlling differentiation, proliferation, maintenance and/or function of the cell types disclosed herein. In addition, novel cell types and methods of quantitating, detecting and isolating the cell types are disclosed.

Embodiments disclosed herein provide a pan-tissue cell atlas from healthy and diseased non-human primates. The atlas was generated using single cell sequencing of tissues obtained from non-human primates (e.g., lymph node, inguinal lymph node, CNS, jejunun, spleen, tonsil, bone marrow, axillary lymph node, colon, ileum, liver, spleen, thymus, brain, lung, stomach or liver). The healthy atlas provides for a map of single cellular composition in healthy tissues and provides mechanisms of homeostasis that specifically correlate to human subjects. Further, the atlas provides for identification of cell-cell interactions between cell types in and between tissues. Specifically, identifying interactions specific to certain tissues, both between immune cells and stroma, identifying heterogeneity between identical “healthy” tissues across individuals, emergent phenotypes of “health,” and identifying ensemble phenotypes that emerge from variable granular details. The atlas also provides tissue specific markers indicating tissue of origin or markers of tissue homing. In certain embodiments, biomarkers can be used to indicate recent emigrants or immigrants. In certain embodiments, recent migrating cells may maintain biomarkers specific to the tissue of origin. Identifying the cell state of these migrating cells may indicate the physiological state of a distant tissue. In certain embodiments, the atlas allows for determining physiological states of a cell or tissue of interest by using the identified correlations between the cells and/or tissues. Thus, the healthy atlas provides cellular biomarkers indicative of the physiological state of another cell or tissue.

Further, a matched disease atlas provides for identification of biomarkers indicative of the physiological state in disease. In certain embodiments, a cross comparison of “matched” cell types between the healthy and disease cell atlases can be used to assess the relative cell frequency and phenotype between the paired tissues. The disease atlas allows for identifying differential coexpression networks of genes in healthy vs. disease. In certain embodiments, using a novel computational and visualization approach is provided for discerning differences between “pathology” and “health.” The disease atlas allows for nominating and testing strategies to “renormalize” tissues from disease to healthy. The disease atlas allows for a comparison of mutational diversity across distinct tissues (e.g., for latent and active SHIV reservoirs). The disease atlas also can be used to infer methods of viral spread in infected individuals, and infer which tissues permit vs inhibit ongoing viral replication.

### Physiological States

The inventors have identified novel markers and networks in non-human primates and further identified correlations between and among those markers in different tissues and cell types. As used herein, the term “correlation” refers to a mutual relationship or connection between cells and/or tissues, in which one cell and/or tissue affects or depends on another cell and/or tissue (e.g., physiological state). As used herein “physiological state” refers to the way in which a living organism, tissue or cell functions, specifically, the condition or state of a cell and/or tissue. Physiological state may also refer to cellular state. Cellular state includes, but is not limited to, gene expression, epigenetic configuration, and nuclear structure. Cells may have a stem-cell like state, different states of differentiation, such as an intermediate state, an immune state (e.g., dysfunctional, effector, naïve, memory state) and a disease state (e.g., infected, malignant state). Tissues can have different states based upon the composition of cells in a microenvironment.

Within the present specification, the terms “differentiation”, “differentiating” or derivatives thereof, denote the process by which an unspecialised or relatively less specialised cell becomes relatively more specialised. In the context of cell ontogeny, the adjective “differentiated” is a relative term. Hence, a “differentiated cell” is a cell that has progressed further down a certain developmental pathway than the cell it is being compared with. The differentiated cell may, for example, be a terminally differentiated cell, i.e., a fully specialised cell capable of taking up specialised functions in various tissues or organs of an organism, which may but need not be post-mitotic; or the differentiated cell may itself be a progenitor cell within a particular differentiation lineage which can further proliferate and/or differentiate.

In certain embodiments, a gene expression profile of one cell correlates with the gene expression profile of a second cell and the correlation is associated with a physiological state. The gene expression profile can include genes that are up and/or downregulated (see, e.g., signature genes described further herein). These markers and correlations can be applied to closely related species. Closely related species can include mammals, primates and humans. As used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans), or any of ape, gibbon, gorilla, chimpanzees orangutan, and macaque. In some embodiments, the mammal may be a mammal of the order Rodentia, such as mice and hamsters. Preferably, the mammal is a non-human primate or a human. An especially preferred mammal is the human.

In certain embodiments, a first cell or tissue may be used as a proxy to measure or otherwise determine the physiological state of second cell or tissue. In a non-limiting example, when a second cell or tissue is not readily accessible, the physiological state of first cell, which may be readily accessible such as by a non-invasive means, can be measured or otherwise determined instead. The inventors have further identified novel markers and networks that overlap between or among non-human primates, normal, or having a disease, disorder, or infection. For example, markers and networks are shown to be comparable between humans and macaques, thus can be used to measure or otherwise determine the physiological state of a cell or tissue in one organism by comparison to a different cell or tissue of another organism. Furthermore, the inventors have shown significant overlap among primates, particularly between Rhesus macaques and humans. Thus, gene and gene cluster expression correlations determined in one organism can be mapped to a second organism. Thus, for example, SHIV-infected macaques are comparable to HIV-infected humans. Likewise, HIV and M. tuberculosis information herein may be applied to non-human primates and other mammals. Conversely, gene expression profiles of model animals may be applied to humans.

Accordingly, the invention provides a method of determining a physiological state of a first cell or tissue in a subject, the method comprising measuring a physiological state of a second cell or tissue in the subject that is correlated with the physiological state of the first cell or tissue. The correlation comprises evaluating gene expression by tissue type, cell type, or tissue type and cell type.

In certain embodiments, the correlation comprises evaluating gene expression by tissue type, cell type, or tissue type and cell type. In an embodiment, the physiological state of the first and second cells or tissues is measured by a gene expression profile comprising one or more genes. In an embodiment of the invention, the physiological state of the first and second cells or tissues is measured by a gene expression profile comprising one or more gene clusters. In certain embodiments, the one or more gene clusters comprise genes having similar function. In certain embodiments, the one or more gene clusters comprise genes that are co-regulated. In certain embodiments, the one or more gene clusters comprise genes of a pathway. In an embodiment, the cells or tissue comprise T cells from mesenteric lymph node, inguinal lymph node, CNS, jejunun, spleen, tonsil, or bone marrow. In an embodiment, the cells or tissue comprise macrophages. In an embodiment of the invention, the cells comprise pneumocytes or NK cells. In an embodiment of the invention the cells comprise cells from axillary lymphnode, colon, ileum, liver, spleen, or thymus.

The invention further provides a method of identifying a biomarker as a proxy for a physiological state of a cell or tissue, the method comprising determining the expression profile of one or more genes in the test cell or tissue, and identifying the expression profile in the test cell or tissue as a proxy for the physiological state of a second cell or tissue if the expression profile in the test cell or tissue is correlated with the expression profile in the second cell or tissue. In an embodiment of the invention, the test cell or tissue is from the same species as the second cell or tissue. In an embodiment of the invention, the test cell or tissue and the second cell or tissue are from a non-human primate. In an embodiment, the test cell or tissue and the second cell or tissue are from a Rhesus macaque. In an embodiment, the test cell or tissue is from a different species as the second cell or tissue. In another embodiment, the test cell or tissue and the second cell or tissue are from different non-human primates. In another embodiment, the test cell or tissue is from a human and the second cell or tissue is from a non-human primate.

The invention further provides a method of diagnosing the physiological state of a cell or tissue in a subject, the method comprising measuring the expression of a biomarker in a test cell or tissue of the subject, wherein the biomarker was identified as a proxy for the physiological state of the diagnosed cell or tissue by determining the expression profile of the biomarker in a first cell or tissue, and identifying the expression profile in the first cell or tissue as a proxy for the physiological state of a second cell or tissue if the expression profile in the first cell or tissue is correlated with the expression profile in the second cell or tissue. The first and second cell or tissue can be from divergent mammal species for genes and gene clusters having similar function and or regulation. In an embodiment, the first cell or tissue is from the same species as the second cell or tissue. In an embodiment, the first cell or tissue and the second cell or tissue are from a non-human primate. In an embodiment, the first cell or tissue and the second cell or tissue are from a Rhesus macaque. In an embodiment, the first cell or tissue is from a different species as the second cell or tissue. In another embodiment, the first cell or tissue and the second cell or tissue are from different non-human primates. In another embodiment, the first cell or tissue is from a human and the second cell or tissue is from a non-human primates.

### Immune States

In certain embodiments, determining an immune state is correlated to a disease state (e.g., HIV or MTB infection). As used herein immune state may also be referred to as an immune response of all the immune cells in an immune system or microenvironment. The immune state may be an immune state correlated with HIV or MTB infection. The immune state may correlate with a diagnosis or prognosis. The immune state may correlate with the ability to infect cells and replicate. In certain embodiments, the immune state may be detected in an immune cell. The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4⁺, CD8⁺, effector Th, memory Th, regulatory Th, CD4⁺/CD8⁺ thymocytes, CD4−/CD8− thymocytes, yδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

As used throughout this specification, “immune response” refers to a response by a cell of the immune system, such as a B cell, T cell (CD4⁺ or CD8⁺), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MEW class I restricted Cytotoxic T lymphocytes (CTLs), may include cytokine and/or cytolytic capabilities, such as lysis of target cells presenting an antigen peptide recognised by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MEW class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; and WO/2017/075478).

The invention also provides compositions and methods for detecting T cell balance, such as the balance between T cell types, e.g., between Th17 and other T cell types, for example, regulatory T cells (Tregs). For example, the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

As used herein, terms such as “pathogenic Th17 cell” and/or “pathogenic Th17 phenotype” and all grammatical variations thereof refer to Th17 cells that, when induced in the presence of TGF-β3, express an elevated level of one or more genes selected from Cxcl3, IL22, IL3, Ccl4, Gzmb, Lrmp, Ccl5, Casp1, Csf2, Ccl3, Tbx21, Icos, IL17r, Stat4, Lgals3 and Lag, as compared to the level of expression in a TGF-β3-induced Th17 cells. As used herein, terms such as “non-pathogenic Th17 cell” and/or “non-pathogenic Th17 phenotype” and all grammatical variations thereof refer to Th17 cells that, when induced in the presence of TGF-β3, express a decreased level of one or more genes selected from IL6st, IL1rn, Ikzf3, Maf, Ahr, IL9 and IL10, as compared to the level of expression in a TGF-β3-induced Th17 cells.

Depending on the cytokines used for differentiation, in vitro polarized Th17 cells can either cause severe autoimmune responses upon adoptive transfer (‘pathogenic Th17 cells’) or have little or no effect in inducing autoimmune disease (‘non-pathogenic cells’) (Ghoreschi et al., 2010; Lee et al., 2012). In vitro differentiation of naïve CD4 T cells in the presence of TGF-β1+IL-6 induces an IL-17A and IL-10 producing population of Th17 cells, that are generally nonpathogenic, whereas activation of naïve T cells in the presence IL-1β+1L-6+IL-23 induces a T cell population that produces IL-17A and IFN-γ, and are potent inducers of autoimmune disease induction (Ghoreschi et al., 2010).

A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p1413-142′7, 3 Dec. 2015; Gaublomme et al., Single-Cell Genomics Unveils Critical Regulators of Th17 Cell Pathogenicity, Cell Volume 163, Issue 6, p1400-1412, 3 Dec. 2015; and International publication numbers WO2016138488A2, WO2015130968, WO/2012/048265, WO/2014/145631 and WO/2014/134351, the contents of which are hereby incorporated by reference in their entirety).

The CD8⁺ T cell response within the tumor microenvironment (TME) is functionally (Sakuishi et al., 2010; Williams et al., 2017; Woo et al., 2012; Xu et al., 2015) and transcriptionally (Singer et al., 2016; Tirosh et al., 2016; Zheng et al., 2017) heterogeneous. At one end of the functional spectrum are CD8⁺ tumor-infiltrating lymphocytes (TILs) that lack the expression of co-inhibitory or immune checkpoint receptors (eg. CTLA-4 and PD-1) and exhibit effector potential, while at the opposite end are CD8⁺ TILs that co-express multiple checkpoint receptors and exhibit an “exhausted” or dysfunctional phenotype.

In certain embodiments, the presence of antigen specific immune cells may be used to detect an immune state. The term “antigen” as used throughout this specification refers to a molecule or a portion of a molecule capable of being bound by an antibody, or by a T cell receptor (TCR) when presented by MHC molecules. At the molecular level, an antigen is characterized by its ability to be bound at the antigen-binding site of an antibody. The specific binding denotes that the antigen will be bound in a highly selective manner by its cognate antibody and not by the multitude of other antibodies which may be evoked by other antigens. An antigen is additionally capable of being recognized by the immune system. In some instances, an antigen is capable of eliciting a humoral immune response in a subject. In some instances, an antigen is capable of eliciting a cellular immune response in a subject, leading to the activation of B- and/or T-lymphocytes. In some instances, an antigen is capable of eliciting a humoral and cellular immune response in a subject. Hence, an antigen may be preferably antigenic and immunogenic. Alternatively, an antigen may be antigenic and not immunogenic. Typically, an antigen may be a peptide, polypeptide, protein, nucleic acid, an oligo- or polysaccharide, or a lipid, or any combination thereof, a glycoprotein, proteoglycan, glycolipid, etc. In certain embodiments, an antigen may be a peptide, polypeptide, or protein. An antigen may have one or more than one epitope. The terms “antigenic determinant” or “epitope” generally refer to the region or part of an antigen that specifically reacts with or is recognized by the immune system, specifically by antibodies, B cells, or T cells.

The term “tumor antigen” as used throughout this specification refers to an antigen that is uniquely or differentially expressed by a tumor cell, whether intracellular or on the tumor cell surface (preferably on the tumor cell surface), compared to a normal or non-neoplastic cell. By means of example, a tumor antigen may be present in or on a tumor cell and not typically in or on normal cells or non-neoplastic cells (e.g., only expressed by a restricted number of normal tissues, such as testis and/or placenta), or a tumor antigen may be present in or on a tumor cell in greater amounts than in or on normal or non-neoplastic cells, or a tumor antigen may be present in or on tumor cells in a different form than that found in or on normal or non-neoplastic cells. The term thus includes tumor-specific antigens (TSA), including tumor-specific membrane antigens, tumor-associated antigens (TAA), including tumor-associated membrane antigens, embryonic antigens on tumors, growth factor receptors, growth factor ligands, etc. The term further includes cancer/testis (CT) antigens. Examples of tumor antigens include, without limitation, (3-human chorionic gonadotropin (βHCG), glycoprotein 100 (gp100/Pme117), carcinoembryonic antigen (CEA), tyrosinase, tyrosinase-related protein 1 (gp75/TRP1), tyrosinase-related protein 2 (TRP-2), NY-BR-1, NY—CO-58, NY-ESO-1, MN/gp250, idiotypes, telomerase, synovial sarcoma X breakpoint 2 (SSX2), mucin 1 (MUC-1), antigens of the melanoma-associated antigen (MAGE) family, high molecular weight-melanoma associated antigen (HMW-MAA), melanoma antigen recognized by T cells 1 (MART1), Wilms' tumor gene 1 (WT1), HER2/neu, mesothelin (MSLN), alphafetoprotein (AFP), cancer antigen 125 (CA-125), and abnormal forms of ras or p53 (see also, WO2016187508A2). Tumor antigens may also be subject specific (e.g., subject specific neoantigens; see, e.g., U.S. Pat. No. 9,115,402; and international patent application publication numbers WO2016100977A1, WO2014168874A2, WO2015085233A1, and WO2015095811A2).

### Disease States

In certain embodiments, the physiological state comprises a disease state. The disease state may include expression of genes in infected cells. The disease state may include a disease microenvironment and the expression of genes in cells within the microenvironment. The disease state may include an immune state. The disease state may include a microenvironment cell state. The disease state may indicate resistance or sensitivity to a treatment. The disease state may indicate the severity of a disease. Diseases or pathogens that lead to a disease state may include, but are not limited to cancer, an autoimmune disease, an inflammatory disease, or an infection (e.g., HIV or MTB, described further herein).

Examples of cancer include but are not limited to carcinoma, lymphoma, blastoma, sarcoma, and leukemia or lymphoid malignancies. More particular examples of such cancers include without limitation: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioma, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as CNS cancer, melanoma, head and neck cancer, bone cancer, bone marrow cancer, duodenum cancer, oesophageal cancer, thyroid cancer, or hematological cancer.

Other non-limiting examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumours, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumours, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumours, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumours, Exocrine Pancreatic Cancer, Extracranial Germ Cell Tumour, Extragonadal Germ Cell Tumour, Extrahepatic Bile Duct Cancer, Eye Cancer, Female Breast Cancer, Gaucher's Disease, Gallbladder Cancer, Gastric Cancer, Gastrointestinal Carcinoid Tumour, Gastrointestinal Tumours, Germ Cell Tumours, Gestational Trophoblastic Tumour, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Hi stiocytoma, Osteosarcoma/Malignant Fibrous Hi stiocytoma of Bone, Ovarian Epithelial Cancer, Ovarian Germ Cell Tumour, Ovarian Low Malignant Potential Tumour, Pancreatic Cancer, Paraproteinemias, Purpura, Parathyroid Cancer, Penile Cancer, Pheochromocytoma, Pituitary Tumour, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumours, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, or Wilms' Tumour.

As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Non-limiting examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

The disease may be an allergic inflammatory disease. The allergic inflammatory disease may be selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome). The asthma may be selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma. The allergy may be to an allergen selected from the group consisting of foods, pollen, mold, dust mites, animals, and animal dander. IBD may comprise a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon. The arthritis may be selected from the group consisting of osteoarthritis, rheumatoid arthritis and psoriatic arthritis.

Examples of pathogenic bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginate, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtherias, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Moraxella catarrhalis, Morganella sp., Mobiluncus sp., Micrococcus sp., Mycobacterium sp. (such as Mycobacterium leprae, Mycobacterium tuberculosis (MTB), Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

In certain example embodiments, the pathogen is a fungus. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, Histoplasma, Mucroymcosis, Pneumocystis, Sporothrix, fungal eye infections ringwork, Exserohilum, and Cladosporium.

In certain example embodiments, the fungus is a yeast. Examples of yeast that can be detected in accordance with disclosed methods include without limitation one or more of (or any combination of), Aspergillus species, a Geotrichum species, a Saccharomyces species, a Hansenula species, a Candida species, a Kluyveromyces species, a Debaryomyces species, a Pichia species, or combination thereof. In certain example embodiments, the fungus is a mold. Example molds include, but are not limited to, a Penicillium species, a Cladosporium species, a Byssochlamys species, or a combination thereof.

In certain example embodiments, the pathogen may be a virus. The virus may be a DNA virus, a RNA virus, or a retrovirus. Example of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV and SHIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zoster virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others.

In certain example embodiments, the pathogen may be a protozoon. Examples of protozoa include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

In certain example embodiments, the physiological state of a microbiota, including commensal microorganism is detected. The Human Microbiome Project sequenced the genome of the human microbiota, focusing particularly on the microbiota that normally inhabit the skin, mouth, nose, digestive tract, and vagina (see, e.g., hmpdacc.org/hmp/).

### Use of Cell Atlas to Determine Cell Interactions

A pan-tissue cell atlas obtained from single subjects may be used to determine connections between tissues and cells in an organism. In certain embodiments, the physiological state of one tissue or cell type may be used as a proxy for determining the physiological state of another tissue or cell. Such correlations between cell types can only be determined using a pan-tissue atlas. The cell atlas may be used as a proxy for tissues or cells in a subject where the tissues or cells are more difficult to obtain. Cell-cell interactions may be identified by determining receptor-ligand expression on interacting cells (see, e.g., Ramilowski et al., 2015, A draft network of ligand-receptor-mediated multicellular signalling in human. Nature Communications volume 6, Article number: 7866). A non-limiting set of ligand receptor pairs is available in the Database of Ligand-Receptor Partners (DLRP) (see, e.g., dip.doe-mbi.ucla.edu/dip/DLRP.cgi?file=dlrp; and Graeber and Eisenberg. Bioinformatic identification of potential autocrine signaling loops in cancers from gene expression profiles. Nature Genetics, 29(3):295-300 (November 2001).

### Biomarkers and Signatures

In certain embodiments, biomarkers are used to indicate a physiological state. The term “biomarker” is widespread in the art and commonly broadly denotes a biological molecule, more particularly an endogenous biological molecule, and/or a detectable portion thereof, whose qualitative and/or quantitative evaluation in a tested object (e.g., in or on a cell, cell population, tissue, organ, or organism, e.g., in a biological sample of a subject) is predictive or informative with respect to one or more aspects of the tested object's phenotype and/or genotype. The terms “marker” and “biomarker” may be used interchangeably throughout this specification. Biomarkers as intended herein may be nucleic acid-based or peptide-, polypeptide- and/or protein-based. For example, a marker may be comprised of peptide(s), polypeptide(s) and/or protein(s) encoded by a given gene, or of detectable portions thereof. Further, whereas the term “nucleic acid” generally encompasses DNA, RNA and DNA/RNA hybrid molecules, in the context of markers the term may typically refer to heterogeneous nuclear RNA (hnRNA), pre-mRNA, messenger RNA (mRNA), or complementary DNA (cDNA), or detectable portions thereof. Such nucleic acid species are particularly useful as markers, since they contain qualitative and/or quantitative information about the expression of the gene. Particularly preferably, a nucleic acid-based marker may encompass mRNA of a given gene, or cDNA made of the mRNA, or detectable portions thereof. Any such nucleic acid(s), peptide(s), polypeptide(s) and/or protein(s) encoded by or produced from a given gene are encompassed by the term “gene product(s)”.

Preferably, markers as intended herein may be extracellular or cell surface markers, as methods to measure extracellular or cell surface marker(s) need not disturb the integrity of the cell membrane and may not require fixation/permeabilization of the cells.

Unless otherwise apparent from the context, reference herein to any marker, such as a peptide, polypeptide, protein, or nucleic acid, may generally also encompass modified forms of said marker, such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “polypeptide” as used throughout this specification generally encompasses polymeric chains of amino acid residues linked by peptide bonds. Hence, insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally-occurring protein parts that ensue from processing of such full-length proteins.

The reference to any marker, including any peptide, polypeptide, protein, or nucleic acid, corresponds to the marker commonly known under the respective designations in the art. The terms encompass such markers of any organism where found, and particularly of animals, preferably warm-blooded animals, more preferably vertebrates, yet more preferably mammals, including humans and non-human mammals, still more preferably of humans.

The terms particularly encompass such markers, including any peptides, polypeptides, proteins, or nucleic acids, with a native sequence, i.e., ones of which the primary sequence is the same as that of the markers found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of markers are intended herein. Accordingly, all sequences of markers found in or derived from nature are considered “native”. The terms encompass the markers when forming a part of a living organism, organ, tissue or cell, when forming a part of a biological sample, as well as when at least partly isolated from such sources. The terms also encompass markers when produced by recombinant or synthetic means.

In certain embodiments, markers, including any peptides, polypeptides, proteins, or nucleic acids, may be human, i.e., their primary sequence may be the same as a corresponding primary sequence of or present in a naturally occurring human markers. Hence, the qualifier “human” in this connection relates to the primary sequence of the respective markers, rather than to their origin or source. For example, such markers may be present in or isolated from samples of human subjects or may be obtained by other means (e.g., by recombinant expression, cell-free transcription or translation, or non-biological nucleic acid or peptide synthesis).

In certain embodiments, markers, including any peptides, polypeptides, proteins, or nucleic acids, may originate from non-human primates, i.e., their primary sequence may be the same as a corresponding primary sequence of or present in a naturally occurring non-human primate markers. Hence, the qualifier “non-human primate” in this connection relates to the primary sequence of the respective markers, rather than to their origin or source. For example, such markers may be present in or isolated from samples of non-human primate subjects or may be obtained by other means (e.g., by recombinant expression, cell-free transcription or translation, or non-biological nucleic acid or peptide synthesis).

The reference herein to any marker, including any peptide, polypeptide, protein, or nucleic acid, also encompasses fragments thereof. Hence, the reference herein to measuring (or measuring the quantity of) any one marker may encompass measuring the marker and/or measuring one or more fragments thereof.

For example, any marker and/or one or more fragments thereof may be measured collectively, such that the measured quantity corresponds to the sum amounts of the collectively measured species. In another example, any marker and/or one or more fragments thereof may be measured each individually. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

The term “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein.

The term “fragment” as used throughout this specification with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of 5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid.

Cells such as central nerve system cells, stem cells, and immune cells as disclosed herein may in the context of the present specification be said to “comprise the expression” or conversely to “not express” one or more markers, such as one or more genes or gene products; or be described as “positive” or conversely as “negative” for one or more markers, such as one or more genes or gene products; or be said to “comprise” a defined “gene or gene product signature”.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells.

### Use of Signatures

The present invention is also directed to signatures and uses thereof. As used herein a “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any gene or genes, protein or proteins, or epigenetic element(s) may be substituted. Reference to a gene name throughout the specification encompasses the human gene, non-human primate gene, mouse gene and all other orthologues as known in the art in other organisms. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory, the signatures can be used to indicate cell-cell interaction in a particular pathological or physiological condition. Not being bound by a theory, the signatures may be indicative of regulatory pathways in immune regulations. Not being bound by a theory, the presence of specific cells and cell subtypes are indicative of a particular response to treatment, such as including increased or decreased susceptibility to treatment. The signature may indicate the presence of one particular cell type.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

In certain embodiments, a signature is characterized as being specific for a particular cell or cell (sub)population state if it is upregulated or only present, detected or detectable in that particular cell or cell (sub)population state (e.g., disease or healthy), or alternatively is downregulated or only absent, or undetectable in that particular cell or cell (sub)population state. In this context, a signature consists of one or more differentially expressed genes/proteins or differential epigenetic elements when comparing different cells or cell (sub)populations, including comparing different gut cell or gut cell (sub)populations, as well as comparing gut cell or gut cell (sub)populations with healthy or disease (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population or subpopulation level, refer to genes that are differentially expressed in all or substantially all cells of the population or subpopulation (such as at least 80%, preferably at least 90%, such as at least 95% of the individual cells). This allows one to define a particular subpopulation of immune cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferably it is meant: induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

In certain embodiments, signature genes and biomarkers related to HIV-infection may be identified by comparing single cell expression profiles obtained from HIV-infected individuals with healthy individuals.

In certain embodiments, signature genes and biomarkers related to HIV-infection may be identified by comparing single cell expression profiles obtained from healthy individuals with cART treated HIV infected individuals. In another embodiment, signature genes and biomarkers related to HIV-infection may be identified by comparing single cell expression profiles obtained from healthy individuals and single cell expression profile from cells obtained from cART treated HIV infected individuals and further reactivated.

In certain embodiments, signature genes and biomarkers related to MTB infection and TB symptoms may be identified by comparing single cell expression profiles obtained from uninfected cells and MTB infected cells.

In one particular embodiment, signature genes and biomarkers related MTB infection and TB symptoms may be identified by comparing single cell expression profiles obtained from uninfected cells and cells infected with detectable copies of MTB, such as MTB strain expressing fluorescence markers.

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In certain example embodiments, the signature genes may be used to distinguish cell types, characterize individual cell phenotypes, cell signatures, cell expression profiles or expression programs, and identify cell-cell interaction in the network of cells within a sampled population present in HIV infected individual or cells based on comparing them to data from bulk analysis of HIV infected sample. In certain example embodiments, the presence of specific immune cells and immune cell subtypes may be indicative of HIV infection, latent HIV infection, and/or resistance to treatment. In certain example embodiments, induction or suppression of specific signature genes may be indicative of HIV infection, latent HIV infection, and/or resistance to treatment. In one example embodiment, detection of one or more signature genes may indicate the presence of a particular cell type or cell types. In certain example embodiments, the presence of immune cell types within HIV infected cell population may indicate that the cells will be sensitive to a treatment.

### Detection of Cell Sub-Populations

In one embodiment, the method comprises detecting or quantifying HIV infected cells in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one embodiment, the method comprises detecting or quantifying a sub-population of cells harboring persistent or latent HIV-infection in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one embodiment, the method comprises detecting or quantifying MTB infected cells in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In one embodiment, the method comprises detecting or quantifying MTB infection state or MTB copy numbers in TB cells in a biological sample. A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

In a preferred embodiment, the method comprises detecting or quantifying pathogen in an easily obtainable sample such as blood or body fluid as a proxy or surrogate indicative of infection states of the tested sub population of cells, a different sub population of cells, a different tissue, or the whole organism.

The terms “increased” or “increase” or “upregulated” or “upregulate” as used herein generally mean an increase by a statically significant amount. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least 50%, or at least 60%, or at least 70%, or at least 80%, at least 90% or more, up to and including a 100% decrease (i.e., absent level as compared to a reference sample), or any decrease between 10-100% as compared to a reference level, as that.

The terms “sample” or “biological sample” as used throughout this specification include any biological specimen obtained from a subject. Particularly useful samples are those known to comprise, or expected or predicted to comprise gut cells as taught herein. Preferably, a sample may be readily obtainable by minimally invasive methods, such as blood collection or tissue biopsy, allowing the removal/isolation/provision of the sample from the subject (e.g., colonoscopy).

The terms “quantity”, “amount” and “level” are synonymous and generally well-understood in the art. The terms as used throughout this specification may particularly refer to an absolute quantification of a marker in a tested object (e.g., in or on a cell, cell population, tissue, organ, or organism, e.g., in a biological sample of a subject), or to a relative quantification of a marker in a tested object, i.e., relative to another value such as relative to a reference value, or to a range of values indicating a base-line of the marker. Such values or ranges may be obtained as conventionally known.

An absolute quantity of a marker may be advantageously expressed as weight or as molar amount, or more commonly as a concentration, e.g., weight per volume or mol per volume. A relative quantity of a marker may be advantageously expressed as an increase or decrease or as a fold-increase or fold-decrease relative to said another value, such as relative to a reference value. Performing a relative comparison between first and second variables (e.g., first and second quantities) may but need not require determining first the absolute values of said first and second variables. For example, a measurement method may produce quantifiable readouts (such as, e.g., signal intensities) for said first and second variables, wherein said readouts are a function of the value of said variables, and wherein said readouts may be directly compared to produce a relative value for the first variable vs. the second variable, without the actual need to first convert the readouts to absolute values of the respective variables.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value >second value; or decrease: first value <second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The term “monitoring” generally refers to the follow-up of a disease or a condition in a subject for any changes which may occur over time.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

### Methods of Detection and Isolation of Cell Types Using Biomarkers

In certain embodiments, the cell types disclosed herein may be detected, quantified or isolated using a technique selected from the group consisting of flow cytometry, mass cytometry, fluorescence activated cell sorting (FACS), fluorescence microscopy, affinity separation, magnetic cell separation, microfluidic separation, RNA-seq (e.g., bulk or single cell), quantitative PCR, MERFISH (multiplex (in situ) RNA FISH) and combinations thereof. The technique may employ one or more agents capable of specifically binding to one or more gene products expressed or not expressed by the gut cells, preferably on the cell surface of the gut cells. The one or more agents may be one or more antibodies. Other methods including absorbance assays and colorimetric assays are known in the art and may be used herein.

Depending on factors that can be evaluated and decided on by a skilled person, such as, inter alia, the type of a marker (e.g., peptide, polypeptide, protein, or nucleic acid), the type of the tested object (e.g., a cell, cell population, tissue, organ, or organism, e.g., the type of biological sample of a subject, e.g., whole blood, plasma, serum, tissue biopsy), the expected abundance of the marker in the tested object, the type, robustness, sensitivity and/or specificity of the detection method used to detect the marker, etc., the marker may be measured directly in the tested object, or the tested object may be subjected to one or more processing steps aimed at achieving an adequate measurement of the marker.

In other example embodiments, detection of a marker may include immunological assay methods, wherein the ability of an assay to separate, detect and/or quantify a marker (such as, preferably, peptide, polypeptide, or protein) is conferred by specific binding between a separable, detectable and/or quantifiable immunological binding agent (antibody) and the marker. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (MA), Western blot, etc.

In certain example embodiments, detection of a marker or signature may include biochemical assay methods, including inter alia assays of enzymatic activity, membrane channel activity, substance-binding activity, gene regulatory activity, or cell signaling activity of a marker, e.g., peptide, polypeptide, protein, or nucleic acid.

In other example embodiments, detection of a marker may include mass spectrometry analysis methods. Generally, any mass spectrometric (MS) techniques that are capable of obtaining precise information on the mass of peptides, and preferably also on fragmentation and/or (partial) amino acid sequence of selected peptides (e.g., in tandem mass spectrometry, MS/MS; or in post source decay, TOF MS), may be useful herein for separation, detection and/or quantification of markers (such as, preferably, peptides, polypeptides, or proteins). Suitable peptide MS and MS/MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 9780121828073) and may be used herein. MS arrangements, instruments and systems suitable for biomarker peptide analysis may include, without limitation, matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)n; atmospheric pressure photoionization mass spectrometry (APPI-MS); APPI-MS/MS; and APPI-(MS)n. Peptide ion fragmentation in tandem MS (MS/MS) arrangements may be achieved using manners established in the art, such as, e.g., collision induced dissociation (CID). Detection and quantification of markers by mass spectrometry may involve multiple reaction monitoring (MRM), such as described among others by Kuhn et al. 2004 (Proteomics 4: 1175-86). MS peptide analysis methods may be advantageously combined with upstream peptide or protein separation or fractionation methods, such as for example with the chromatographic and other methods.

In other example embodiments, detection of a marker may include chromatography methods. In a one example embodiment, chromatography refers to a process in which a mixture of substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography may be columnar. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilised metal affinity chromatography, and the like.

In certain embodiments, further techniques for separating, detecting and/or quantifying markers may be used in conjunction with any of the above described detection methods. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), capillary gel electrophoresis (CGE), capillary zone electrophoresis (CZE), micellar electrokinetic chromatography (MEKC), free flow electrophoresis (FFE), etc.

In certain examples, such methods may include separating, detecting and/or quantifying markers at the nucleic acid level, more particularly RNA level, e.g., at the level of hnRNA, pre-mRNA, mRNA, or cDNA. Standard quantitative RNA or cDNA measurement tools known in the art may be used. Non-limiting examples include hybridization-based analysis, microarray expression analysis, digital gene expression profiling (DGE), RNA-in-situ hybridization (RISH), Northern-blot analysis and the like; PCR, RT-PCR, RT-qPCR, end-point PCR, digital PCR or the like; supported oligonucleotide detection, pyrosequencing, polony cyclic sequencing by synthesis, simultaneous bi-directional sequencing, single-molecule sequencing, single molecule real time sequencing, true single molecule sequencing, hybridization-assisted nanopore sequencing, sequencing by synthesis, single-cell RNA sequencing (sc-RNA seq), or the like.

### Single Cell Sequencing

By means of an example, methods to profile the RNA content of large numbers of individual cells have been recently developed. The cell of origin is determined by a cellular barcode. In certain embodiments, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-6′73, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain example embodiments, using Seq-Well for massively parallel scRNA-seq (Shalek reference Re: Seq-well) of surgical resections from individuals infected by HIV (HIV+) and healthy individuals (HIV−), cells and tissues representative of infection states were located, and biomarkers related to (latent) infection in specific cells were identified.

In certain example embodiment, using Seq-Well for massively parallel scRNA-seq of surgical resections from individuals infected by MTB (MTB+) and healthy individuals (MTB−), cells and tissues representative of infection states were located, and biomarkers related to (latent) infection in specific cells were identified.

In certain example embodiments, using Seq-Well for massively parallel scRNA-seq (Gierahn et al., 2017) of surgical resections from individuals infected by HIV (HIV+) and healthy individuals (HIV−), cells and tissues representative of infection states were located, and biomarkers related to (latent) infection in specific cells were identified.

In certain example embodiment, using Seq-Well for massively parallel scRNA-seq of surgical resections from individuals infected by MTB (MTB+) and healthy individuals (MTB−), cells and tissues representative of infection states were located, and biomarkers related to (latent) infection in specific cells were identified.

### Isolating Cells

In certain embodiments, a first cell type or test cell is isolated from a subject. In certain embodiments, immune cells may be obtained using any method known in the art. In one embodiment, allogenic immune cells may be obtained from healthy subjects. In one embodiment, immune cells that have infiltrated a tumor are isolated. immune cells may be removed during surgery. immune cells may be isolated after removal of tumor tissue by biopsy. immune cells may be isolated by any means known in the art. In one embodiment, immune cells are obtained by apheresis. In one embodiment, the method may comprise obtaining a bulk population of immune cells from a tumor sample by any suitable method known in the art. For example, a bulk population of immune cells can be obtained from a tumor sample by dissociating the tumor sample into a cell suspension from which specific cell populations can be selected. Suitable methods of obtaining a bulk population of immune cells may include, but are not limited to, any one or more of mechanically dissociating (e.g., mincing) the tumor, enzymatically dissociating (e.g., digesting) the tumor, and aspiration (e.g., as with a needle).

Immune cells can be obtained from a number of sources, including peripheral blood mononuclear cells (PBMC), bone marrow, lymph node tissue, spleen tissue, and tumors. In certain embodiments of the present invention, immune cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted or isolated from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such isolation or depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

Immune cells can also be frozen for later analysis. In certain embodiments, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MEW tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-WIC tetramers can be generated using techniques known in the art and can be made with any WIC molecule of interest and any antigen of interest as described herein. Specific epitopes to be used in this context can be identified using numerous assays known in the art. For example, the ability of a polypeptide to bind to WIC class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

The terms “isolating” or “purifying” as used throughout this specification with reference to a particular component of a composition or mixture (e.g., the tested object such as the biological sample) encompass processes or techniques whereby such component is separated from one or more or (substantially) all other components of the composition or mixture (e.g., the tested object such as the biological sample). The terms do not require absolute purity. Instead, isolating or purifying the component will produce a discrete environment in which the abundance of the component relative to one or more or all other components is greater than in the starting composition or mixture (e.g., the tested object such as the biological sample). A discrete environment may denote a single medium, such as for example a single solution, dispersion, gel, precipitate, etc. Isolating or purifying the specified cells from the tested object such as the biological sample may increase the abundance of the specified cells relative to all other cells comprised in the tested object such as the biological sample, or relative to other cells of a select subset of the cells comprised in the tested object such as the biological sample, e.g., relative to other white blood cells, peripheral blood mononuclear cells, immune cells, antigen presenting cells, or dendritic cells comprised in the tested object such as the biological sample. By means of example, isolating or purifying the specified cells from the tested object such as the biological sample may yield a cell population, in which the specified cells constitute at least 40% (by number) of all cells of said cell population, for example, at least 45%, preferably at least 50%, at least 55%, more preferably at least 60%, at least 65%, still more preferably at least 70%, at least 75%, even more preferably at least 80%, at least 85%, and yet more preferably at least 90%, at least 95%, at least 96%, at least 97%, at least 98%, at least 99%, or even 100% of all cells of said cell population.

The method may allow a skilled person to detect or conclude the presence or absence of the specified cells in a tested object (e.g., in a cell population, tissue, organ, organism, or in a biological sample of a subject). The method may also allow a skilled person to quantify the specified cells in a tested object (e.g., in a cell population, tissue, organ, organism, or in a biological sample of a subject). The quantity of the specified cells in the tested object such as the biological sample may be suitably expressed for example as the number (count) of the specified immune cells per standard unit of volume (e.g., ml, μA or nl) or weight (e.g., g or mg or ng) of the tested object such as the biological sample. The quantity of the specified cells in the tested object such as the biological sample may also be suitably expressed as a percentage or fraction (by number) of all cells comprised in the tested object such as the biological sample, or as a percentage or fraction (by number) of a select subset of the cells comprised in the tested object such as the biological sample, e.g., as a percentage or fraction (by number) of white blood cells, peripheral blood mononuclear cells, immune cells, antigen presenting cells, or dendritic cells comprised in the tested object such as the biological sample. The quantity of the specified cells in the tested object such as the biological sample may also be suitably represented by an absolute or relative quantity of a suitable surrogate analyte, such as a peptide, polypeptide, protein, or nucleic acid expressed or comprised by the specified cells.

Where a marker is detected in or on a cell, the cell may be conventionally denoted as positive (+) or negative (−) for the marker. Semi-quantitative denotations of marker expression in cells are also commonplace in the art, such as particularly in flow cytometry quantifications, for example, “dim” vs. “bright”, or “low” vs. “medium”/“intermediate” vs. “high”, or “−” vs. “⁺” vs. “⁺⁺”, commonly controlled in flow cytometry quantifications by setting of the gates. Where a marker is quantified in or on a cell, absolute quantity of the marker may also be expressed for example as the number of molecules of the marker comprised by the cell.

Where a marker is detected and/or quantified on a single cell level in a cell population, the quantity of the marker may also be expressed as a percentage or fraction (by number) of cells comprised in said population that are positive for said marker, or as percentages or fractions (by number) of cells comprised in said population that are “dim” or “bright”, or that are “low” or “medium”/“intermediate” or “high”, or that are “−” or “⁺” or “⁺⁺”. By means of an example, a sizeable proportion of the tested cells of the cell population may be positive for the marker, e.g., at least about 20%, at least about 40%, at least about 50%, at least about 60%, at least about 70%, at least about 80%, at least about 90%, at least about 95%, or up to 100%.

### Use of Specific Binding Agents

In certain embodiments, the aforementioned methods and techniques may employ agent(s) capable of specifically binding to one or more gene products, e.g., peptides, polypeptides, proteins, or nucleic acids, expressed or not expressed by the cells as taught herein. In certain preferred embodiments, such one or more gene products, e.g., peptides, polypeptides, or proteins, may be expressed on the cell surface of the immune cells (i.e., cell surface markers, e.g., transmembrane peptides, polypeptides or proteins, or secreted peptides, polypeptides or proteins which remain associated with the cell surface). Hence, further disclosed are binding agents capable of specifically binding to markers, such as genes or gene products, e.g., peptides, polypeptides, proteins, or nucleic acids as taught herein. Binding agents as intended throughout this specification may include inter alia antibodies, aptamers, spiegelmers (L-aptamers), photoaptamers, protein, peptides, peptidomimetics, nucleic acids such as oligonucleotides (e.g., hybridization probes or amplification or sequencing primers and primer pairs), small molecules, or combinations thereof.

The term “aptamer” refers to single-stranded or double-stranded oligo-DNA, oligo-RNA or oligo-DNA/RNA or any analogue thereof that specifically binds to a target molecule such as a peptide. Advantageously, aptamers display fairly high specificity and affinity (e.g., KA in the order 1×109 M-1) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule. The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

Binding agents may be in various forms, e.g., lyophilised, free in solution, or immobilised on a solid phase. They may be, e.g., provided in a multi-well plate or as an array or microarray, or they may be packaged separately, individually, or in combination.

The term “specifically bind” as used throughout this specification means that an agent (denoted herein also as “specific-binding agent”) binds to one or more desired molecules or analytes (e.g., peptides, polypeptides, proteins, or nucleic acids) substantially to the exclusion of other molecules which are random or unrelated, and optionally substantially to the exclusion of other molecules that are structurally related. The term “specifically bind” does not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold, or at least about 1000-fold, or at least about 104-fold, or at least about 105-fold, or at least about 106-fold or more greater, than its affinity for a non-target molecule, such as for a suitable control molecule (e.g., bovine serum albumin, casein).

Preferably, the specific binding agent may bind to its intended target(s) with affinity constant (K_{A}) of such binding K_{A}≥1λ10⁶ M⁻¹, more preferably K_{A}≥1λ10⁷ M⁻¹, yet more preferably K_{A}≥1×10⁸ M⁻¹, even more preferably K_{A}≥1×10⁹ M⁻¹, and still more preferably K_{A}≥1×10¹⁰ M⁻¹ or K_{A}≥1×10¹¹ M⁻¹ or K_{A}≥1×10¹² M⁻¹, wherein K_{A}=[SBA_T]/[SBA][T], SBA denotes the specific-binding agent, T denotes the intended target. Determination of K_{A} can be carried out by methods known in the art, such as for example, using equilibrium dialysis and Scatchard plot analysis.

In certain embodiments, the one or more binding agents may be one or more antibodies. As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest, i.e., antigen-binding fragments), as well as multivalent and/or multi-specific composites of such fragments. The term “antibody” is not only inclusive of antibodies generated by methods comprising immunization, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo. Antibodies also encompasses chimeric, humanized and fully humanized antibodies.

An antibody may be any of IgA, IgD, IgE, IgG and IgM classes, and preferably IgG class antibody. An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

Antibody binding agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments, single domain (sd) Fv, such as VH domains, VL domains and VHH domains; diabodies; linear antibodies; single-chain antibody molecules, in particular heavy-chain antibodies; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Nucleic acid binding agents, such as oligonucleotide binding agents, are typically at least partly antisense to a target nucleic acid of interest. The term “antisense” generally refers to an agent (e.g., an oligonucleotide) configured to specifically anneal with (hybridise to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein, such as hybridisation probes or amplification or sequencing primers and primer pairs) may typically be capable of annealing with (hybridizing to) the respective target nucleic acid sequences at high stringency conditions, and capable of hybridising specifically to the target under physiological conditions. The terms “complementary” or “complementarity” as used throughout this specification with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The reference to oligonucleotides may in particular but without limitation include hybridization probes and/or amplification primers and/or sequencing primers, etc., as commonly used in nucleic acid detection technologies.

Binding agents as discussed herein may suitably comprise a detectable label. The term “label” refers to any atom, molecule, moiety or biomolecule that may be used to provide a detectable and preferably quantifiable read-out or property, and that may be attached to or made part of an entity of interest, such as a binding agent. Labels may be suitably detectable by for example mass spectrometric, spectroscopic, optical, colourimetric, magnetic, photochemical, biochemical, immunochemical or chemical means. Labels include without limitation dyes; radiolabels such as ³²P, ³³P, ³⁵S, ¹²⁵I, ¹³¹I; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; and fluorescent dyes alone or in combination with moieties that may suppress or shift emission spectra by fluorescence resonance energy transfer (FRET).

In some embodiments, binding agents may be provided with a tag that permits detection with another agent (e.g., with a probe binding partner). Such tags may be, for example, biotin, streptavidin, his-tag, myc tag, maltose, maltose binding protein or any other kind of tag known in the art that has a binding partner. Example of associations which may be utilised in the probe:binding partner arrangement may be any, and includes, for example biotin: streptavidin, his-tag:metal ion (e.g., Ni2⁺), maltose:maltose binding protein, etc.

The marker-binding agent conjugate may be associated with or attached to a detection agent to facilitate detection. Examples of detection agents include, but are not limited to, luminescent labels; colourimetric labels, such as dyes; fluorescent labels; or chemical labels, such as electroactive agents (e.g., ferrocyanide); enzymes; radioactive labels; or radiofrequency labels. The detection agent may be a particle. Examples of such particles include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal (hydrous) oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads. Preferable particles may be colloidal gold particles.

In certain embodiments, the one or more binding agents are configured for use in a technique selected from the group consisting of flow cytometry, fluorescence activated cell sorting, mass cytometry, fluorescence microscopy, affinity separation, magnetic cell separation, microfluidic separation, and combinations thereof.

### Modulating Agents

In an embodiment, the invention provides a method of determining the effect of a modulating agent on a first cell or tissue in a subject, the method comprising measuring the effect of the modulating agent on a second cell or tissue in the subject, wherein the physiological state of the second cell or tissue is correlated with the effect of the modulating agent on the first cell or tissue. In certain embodiments, the agent is a therapeutic agent. For example, an immunotherapy may be administered to a subject having an aberrant immune response in a tissue difficult to obtain cells from (e.g., IBD in the gut or a tumor in the brain). The effect of the immunotherapy in the tissue may be determined by correlating the effect on circulating immune cells.

A further aspect of the invention relates to a method for identifying an agent capable of modulating one or more phenotypic aspects of a cell or tissue (e.g., a healthy phenotype, immune cell and/or tissue, tumor microenvironment, pathogen infected cell, comprising: determining an expression profile of one or more genes in a test cell or tissue obtained from an organism treated with the modulating agent that correlates with the expression profile in a second cell or tissue obtained from the treated organism. In another aspect, a method for identifying an agent capable of modulating one or more phenotypic aspects of a cell that has a physiological state that correlates with a second cell comprising a) applying a candidate agent to the cell or cell population; b) detecting modulation of one or more phenotypic aspects of the cell or cell population that correlates with the phenotype in the second cell by the candidate agent, thereby identifying the agent.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of a gut cell or gut cell population may be modulated without substantially altering other (unintended, undesired) phenotypic aspect(s).

The term “agent” broadly encompasses any condition, substance or agent capable of modulating one or more phenotypic aspects of cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the gut cell or gut cell population (e.g., exposing the gut cell or gut cell population to the candidate agent or contacting the gut cell or gut cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof.

In certain embodiments, the present invention provides for one or more therapeutic agents or combinations of agents. In certain embodiments, the agents target correlating cells or tissues or a target cell or tissue. Targeting the cells or tissues may provide for enhanced or otherwise previously unknown activity in the treatment of disease. In certain embodiments, an agent against a target may already be known or used clinically. In certain embodiments, the agents are used to modulate cell types. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried. For example, in methods for treating cancer in a subject, an effective amount of a an agent is any amount that provides an anti-cancer effect, such as reduces or prevents proliferation of a cancer cell or is cytotoxic towards a cancer cell.

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). Specific small molecule degraders targeting bromodomain and extra-terminal (BET) family proteins, consisting of BRD2, BRD3, BRD4, and testis-specific BRDT members (e.g., BETd-260/ZBC260) are specifically applicable for targeting the identified synthetic lethal combinations comprising BRD4 (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481).

As described herein, small molecules targeting epigenetic proteins are currently being developed and/or used in the clinic to treat disease (see, e.g., Qi et al., HEDD: the human epigenetic drug database. Database, 2016, 1-10; and Ackloo et al., Chemical probes targeting epigenetic proteins: Applications beyond oncology. Epigenetics 2017, VOL. 12, NO. 5, 378-400). In certain embodiments, the one or more agents comprise a histone acetylation inhibitor, histone deacetylase (HDAC) inhibitor, histone lysine methylation inhibitor, histone lysine demethylation inhibitor, DNA methyltransferase (DNMT) inhibitor, inhibitor of acetylated histone binding proteins, inhibitor of methylated histone binding proteins, sirtuin inhibitor, protein arginine methyltransferase inhibitor or kinase inhibitor. In certain embodiments, any small molecule exhibiting the functional activity described above may be used in the present invention. In certain embodiments, the DNA methyltransferase (DNMT) inhibitor is selected from the group consisting of azacitidine (5-azacytidine), decitabine (5-aza-2′-deoxycytidine), EGCG (epigallocatechin-3-gallate), zebularine, hydralazine, and procainamide. In certain embodiments, the histone acetylation inhibitor is C646. In certain embodiments, the histone deacetylase (HDAC) inhibitor is selected from the group consisting of vorinostat, givinostat, panobinostat, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, abexinostat, resminostat, dacinostat, droxinostat, and trichostatin A (TSA). In certain embodiments, the histone lysine demethylation inhibitor is selected from the group consisting of pargyline, clorgyline, bizine, GSK2879552, GSK-J4, KDMS-C70, JIB-04, and tranylcypromine. In certain embodiments, the histone lysine methylation inhibitor is selected from the group consisting of EPZ-6438, GSK126, CPI-360, CPI-1205, CPI-0209, DZNep, GSK343, EIL BIX-01294, UNC0638, EPZ004777, GSK343, UNC1999 and UNC0224. In certain embodiments, the inhibitor of acetylated histone binding proteins is selected from the group consisting of AZD5153 (see e.g., Rhyasen et al., AZD5153: A Novel Bivalent BET Bromodomain Inhibitor Highly Active against Hematologic Malignancies, Mol Cancer Ther. 2016 November; 15(11):2563-2574. Epub 2016 Aug. 29), PFI-1, CPI-203, CPI-0610, RVX-208, OTX015, I-BET151, I-BET762, I-BET-726, dBET1, ARV-771, ARV-825, BETd-260/ZBC260 and MZ1. In certain embodiments, the inhibitor of methylated histone binding proteins is selected from the group consisting of UNC669 and UNC1215. In certain embodiments, the sirtuin inhibitor comprises nicotinamide.

In certain embodiments, the agent reactivates latent HIV or SHIV. In certain embodiments, the agent comprises phorbol myristate acetate (PMA) with or without ionomycin, or PHA/IL2.

In certain embodiments, the agent is an immunotherapy (e.g., checkpoint inhibitors, CAR T cells). Immunotherapies have been developed to enhance immune responses against cancer and lead to prolonged survival. Immune checkpoint inhibitors (ICI) have transformed the therapeutic landscape of several cancer types (Sharma and Allison, 2015 The future of immune checkpoint therapy. Science 348, 56-61). In particular, immune checkpoint inhibitors (ICI) lead to durable responses in ˜35% of patients with metastatic melanoma by unleashing T cells from oncogenic suppression (Sharma, et al., 2015; and Hodi, et al., 2016 Durable, long-term survival in previously treated patients with advanced melanoma who received nivolumab monotherapy in a phase I trial. 2016 AACR Annu. Meet. Abstr. CT001 Present. Apr. 17 2016). Nonetheless, many patients manifest ICI resistance (ICR), which is often intrinsic (Sharma et al., 2017 Primary, Adaptive, and Acquired Resistance to Cancer Immunotherapy. Cell 168, 707-723). ICR is often unpredictable and poorly understood (Sharma, et al., 2017), hampering appropriate selection of patients for therapies, rational enrollment to clinical trials and the development of new therapeutic strategies that could overcome ICR (Sharma, et al., 2015). The checkpoint blockade therapy may comprise anti-TIM3, anti-CTLA4, anti-PD-L1, anti-PD1, anti-TIGIT, anti-LAG3, or combinations thereof. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab).

By means of example but without limitation, agents can include low molecular weight compounds, but may also be larger compounds, or any organic or inorganic molecule effective in the given situation, including modified and unmodified nucleic acids such as antisense nucleic acids, RNAi, such as siRNA or shRNA, CRISPR/Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments, the agent is a small molecule having a chemical moiety. Agents can be known to have a desired activity and/or property, or can be selected from a library of diverse compounds.

In certain embodiments, an agent may be a hormone, a cytokine, a lymphokine, a growth factor, a chemokine, a cell surface receptor ligand such as a cell surface receptor agonist or antagonist, or a mitogen.

Non-limiting examples of hormones include growth hormone (GH), adrenocorticotropic hormone (ACTH), dehydroepiandrosterone (DHEA), cortisol, epinephrine, thyroid hormone, estrogen, progesterone, testosterone, or combinations thereof.

Non-limiting examples of cytokines include lymphokines (e.g., interferon-γ, IL-2, IL-3, IL-4, IL-6, granulocyte-macrophage colony-stimulating factor (GM-CSF), interferon-γ, leukocyte migration inhibitory factors (T-LIF, B-LIF), lymphotoxin-alpha, macrophage-activating factor (MAF), macrophage migration-inhibitory factor (MIF), neuroleukin, immunologic suppressor factors, transfer factors, or combinations thereof), monokines (e.g., IL-1, TNF-alpha, interferon-α, interferon-β, colony stimulating factors, e.g., CSF2, CSF3, macrophage CSF or GM-CSF, or combinations thereof), chemokines (e.g., beta-thromboglobulin, C chemokines, CC chemokines, CXC chemokines, CX3C chemokines, macrophage inflammatory protein (MIP), or combinations thereof), interleukins (e.g., IL-1, IL-2, IL-3, IL-4, IL-5, IL-6, IL-7, IL-8, IL-9, IL-10, IL-11, IL-12, IL-13, IL-14, IL-15, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof), and several related signaling molecules, such as tumour necrosis factor (TNF) and interferons (e.g., interferon-α, interferon-β, interferon-γ, interferon-λ, or combinations thereof).

Non-limiting examples of growth factors include those of fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, or combinations thereof.

Non-limiting examples of mitogens include phytohaemagglutinin (PHA), concanavalin A (conA), lipopolysaccharide (LPS), pokeweed mitogen (PWM), phorbol ester such as phorbol myristate acetate (PMA) with or without ionomycin, or combinations thereof.

Non-limiting examples of cell surface receptors the ligands of which may act as agents include Toll-like receptors (TLRs) (e.g., TLR1, TLR2, TLR3, TLR4, TLR5, TLR6, TLR7, TLR8, TLR9, TLR10, TLR11, TLR12 or TLR13), CD80, CD86, CD40, CCR7, or C-type lectin receptors.

Particular screening applications of this invention relate to the testing of pharmaceutical compounds in drug research. The reader is referred generally to the standard textbook In vitro Methods in Pharmaceutical Research, Academic Press, 1997, and U.S. Pat. No. 5,030,015. In certain aspects of this invention, the culture of the invention is used to grow and differentiate a cachectic target cell to play the role of test cells for standard drug screening and toxicity assays. Assessment of the activity of candidate pharmaceutical compounds generally involves combining the target cell (e.g., a myocyte, an adipocyte, a cardiomyocyte or a hepatocyte) with the candidate compound, determining any change in the morphology, marker phenotype, or metabolic activity of the cells that is attributable to the candidate compound (compared with untreated cells or cells treated with an inert compound, such as vehicle), and then correlating the effect of the candidate compound with the observed change. The screening may be done because the candidate compound is designed to have a pharmacological effect on the target cell, or because a candidate compound may have unintended side effects on the target cell. Alternatively, libraries can be screened without any predetermined expectations in hopes of identifying compounds with desired effects.

Cytotoxicity can be determined in the first instance by the effect on cell viability and morphology. In certain embodiments, toxicity may be assessed by observation of vital staining techniques, ELISA assays, immunohistochemistry, and the like or by analyzing the cellular content of the culture, e.g., by total cell counts, and differential cell counts or by metabolic markers such as MTT and XTT.

Additional further uses of the culture of the invention include, but are not limited to, its use in research e.g., to elucidate mechanisms leading to the identification of novel targets for therapies, and to generate genotype-specific cells for disease modeling, including the generation of new therapies customized to different genotypes. Such customization can reduce adverse drug effects and help identify therapies appropriate to the patient's genotype.

In certain embodiments, the present invention provides method for high-throughput screening. “High-throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus, “high-throughput screening” as used herein does not include handling large quantities of radioactive materials, slow and complicated operator-dependent screening steps, and/or prohibitively expensive reagent costs, etc.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures of the present invention may be used to screen for drugs that induce or reduce the signature in immune cells as described herein. The signature may be used for GE-HTS (Gene Expression-based High-Throughput Screening). In certain embodiments, pharmacological screens may be used to identify drugs that selectively activate gut cells.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature of the present invention in silico.

### Genetic Modification

In certain embodiments, one or more endogenous genes may be modified using a nuclease. The term “nuclease” as used herein broadly refers to an agent, for example a protein or a small molecule, capable of cleaving a phosphodiester bond connecting nucleotide residues in a nucleic acid molecule. In some embodiments, a nuclease may be a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. Preferably, the nuclease is an endonuclease. Preferably, the nuclease is a site-specific nuclease, binding and/or cleaving a specific phosphodiester bond within a specific nucleotide sequence, which may be referred to as “recognition sequence”, “nuclease target site”, or “target site”. In some embodiments, a nuclease may recognize a single stranded target site, in other embodiments a nuclease may recognize a double-stranded target site, for example a double-stranded DNA target site. Some endonucleases cut a double-stranded nucleic acid target site symmetrically, i.e., cutting both strands at the same position so that the ends comprise base-paired nucleotides, also known as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs”, e.g., “5′-overhang” or “3′-overhang”, depending on whether the unpaired nucleotide(s) form(s) the 5′ or the 5′ end of the respective DNA strand.

The nuclease may introduce one or more single-strand nicks and/or double-strand breaks in the endogenous gene, whereupon the sequence of the endogenous gene may be modified or mutated via non-homologous end joining (NHEJ) or homology-directed repair (HDR).

In certain embodiments, the nuclease may comprise (i) a DNA-binding portion configured to specifically bind to the endogenous gene and (ii) a DNA cleavage portion. Generally, the DNA cleavage portion will cleave the nucleic acid within or in the vicinity of the sequence to which the DNA-binding portion is configured to bind.

In certain embodiments, the DNA-binding portion may comprise a zinc finger protein or DNA-binding domain thereof, a transcription activator-like effector (TALE) protein or DNA-binding domain thereof, or an RNA-guided protein or DNA-binding domain thereof.

In certain embodiments, the DNA-binding portion may comprise (i) Cas9 or Cpf1 or any Cas protein described herein modified to eliminate its nuclease activity, or (ii) DNA-binding domain of Cas9 or Cpf1 or any Cas protein described herein.

In certain embodiments, the DNA cleavage portion comprises Fold or variant thereof or DNA cleavage domain of Fold or variant thereof.

In certain embodiments, the nuclease may be an RNA-guided nuclease, such as Cas9 or Cpf1 or any Cas protein described herein.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B 1 and EP 2 771 468 B 1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 Jun. 10, 2014; PCT/US2014/041808 filed Jun. 11, 2014; and PCT/US2014/62558 filed Oct. 28, 2014, and U.S. Provisional Patent Applications Ser. Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to US provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert BL, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al., “Cpf1 Is a Single RNA-Guided Endonuclease of a Class 2 CRISPR-Cas System,” Cell 163, 1-13 (Oct. 22, 2015)
Shmakov et al., “Discovery and Functional Characterization of Diverse Class 2 CRISPR-Cas Systems,” Molecular Cell 60, 1-13 (Available online Oct. 22, 2015)

each of which is incorporated herein by reference, may be considered in the practice of the instant invention, and discussed briefly below:

Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.

Konermann et al. (2013) addressed the need in the art for versatile and robust technologies that enable optical and chemical modulation of DNA-binding domains based CRISPR Cas9 enzyme and also Transcriptional Activator Like Effectors

Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.

Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.

Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.

Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.

Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.

Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.

Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.

Hsu et al. (2014) is a review article that discusses generally CRISPR-Cas9 history from yogurt to genome editing, including genetic screening of cells.

Wang et al. (2014) relates to a pooled, loss-of-function genetic screening approach suitable for both positive and negative selection that uses a genome-scale lentiviral single guide RNA (sgRNA) library.

Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

Swiech et al. demonstrate that AAV-mediated SpCas9 genome editing can enable reverse genetic studies of gene function in the brain.

Konermann et al. (2015) discusses the ability to attach multiple effector domains, e.g., transcriptional activator, functional and epigenomic regulators at appropriate positions on the guide such as stem or tetraloop with and without linkers.

Zetsche et al. demonstrates that the Cas9 enzyme can be split into two and hence the assembly of Cas9 for activation can be controlled.

Chen et al. relates to multiplex screening by demonstrating that a genome-wide in vivo CRISPR-Cas9 screen in mice reveals genes regulating lung metastasis.

Ran et al. (2015) relates to SaCas9 and its ability to edit genomes and demonstrates that one cannot extrapolate from biochemical assays.

Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.

Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.

Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.

Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.

Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Zetsche et al. (2015) reported the characterization of Cpf1, a putative class 2 CRISPR effector. It was demonstrated that Cpf1 mediates robust DNA interference with features distinct from Cas9. Identifying this mechanism of interference broadens our understanding of CRISPR-Cas systems and advances their genome editing applications.

Shmakov et al. (2015) reported the characterization of three distinct Class 2 CRISPR-Cas systems. The effectors of two of the identified systems, C2c1 and C2c3, contain RuvC like endonuclease domains distantly related to Cpf1. The third system, C2c2, contains an effector with two predicted HEPN RNase domains.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided Fold Nucleases that recognize extended sequences and can edit endogenous genes with high efficiencies in human cells.

In general, the CRISPR-Cas or CRISPR system is as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention the terms guide sequence and guide RNA, i.e. RNA capable of guiding Cas to a target genomic locus, are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In a classic CRISPR-Cas system, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In particularly preferred embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence.

The methods according to the invention as described herein comprehend inducing one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it will be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

The nucleic acid molecule encoding a Cas is advantageously codon optimized Cas. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also, the way how the Cas transgene is introduced in the cell is may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox (LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus, such as for instance one or more oncogenic mutations, as for instance and without limitation described in Platt et al. (2014), Chen et al., (2014) or Kumar et al. (2009).

In some embodiments, the Cas sequence is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Cas comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 1); the NLS from nucleoplasmin (e.g. the nucleoplasmin bipartite NLS with the sequence KRPAATKKAGQAKKKK) (SEQ ID NO: 2); the c-myc NLS having the amino acid sequence PAAKRVKLD (SEQ ID NO: 3) or RQRRNELKRSP (SEQ ID NO: 4); the hRNPA1 M9 NLS having the sequence NQSSNFGPMKGGNFGGRSSGPYGGGGQYFAKPRNQGGY(SEQ ID NO: 5); the sequence RMRIZFKNKGKDTAELRRRRVEVSVELRKAKKDEQILKRRNV (SEQ ID NO: 6) of the IBB domain from importin-alpha; the sequences VSRKRPRP (SEQ ID NO: 7) and PPKKARED (SEQ ID NO: 8) of the myoma T protein; the sequence POPKKKPL (SEQ ID NO: 9) of human p53; the sequence SALIKKKKKMAP (SEQ ID NO: 10) of mouse c-abl IV; the sequences DRLRR (SEQ ID NO: 11) and PKQKKRK (SEQ ID NO: 12) of the influenza virus NS1; the sequence RKLKKKIKKL (SEQ ID NO: 13) of the Hepatitis virus delta antigen; the sequence REKKKFLKRR (SEQ ID NO: 14) of the mouse Mx1 protein; the sequence KRKGDEVDGVDEVAKKKSKK (SEQ ID NO: 15) of the human poly(ADP-ribose) polymerase; and the sequence RKCLQAGMNLEARKTKK (SEQ ID NO: 16) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs.

### Zinc Finger and TALE

One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In advantageous embodiments of the invention, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), monomers with an RVD of NG preferentially bind to thymine (T), monomers with an RVD of HD preferentially bind to cytosine (C) and monomers with an RVD of NN preferentially bind to both adenine (A) and guanine (G). In yet another embodiment of the invention, monomers with an RVD of IG preferentially bind to T. Thus, the number and order of the polypeptide monomer repeats in the nucleic acid binding domain of a TALE determines its nucleic acid target specificity. In still further embodiments of the invention, monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, monomers having RVDs of H*, HA, KA, N*, NA, NC, NS, RA, and S* bind to adenine, guanine, cytosine and thymine with comparable affinity.

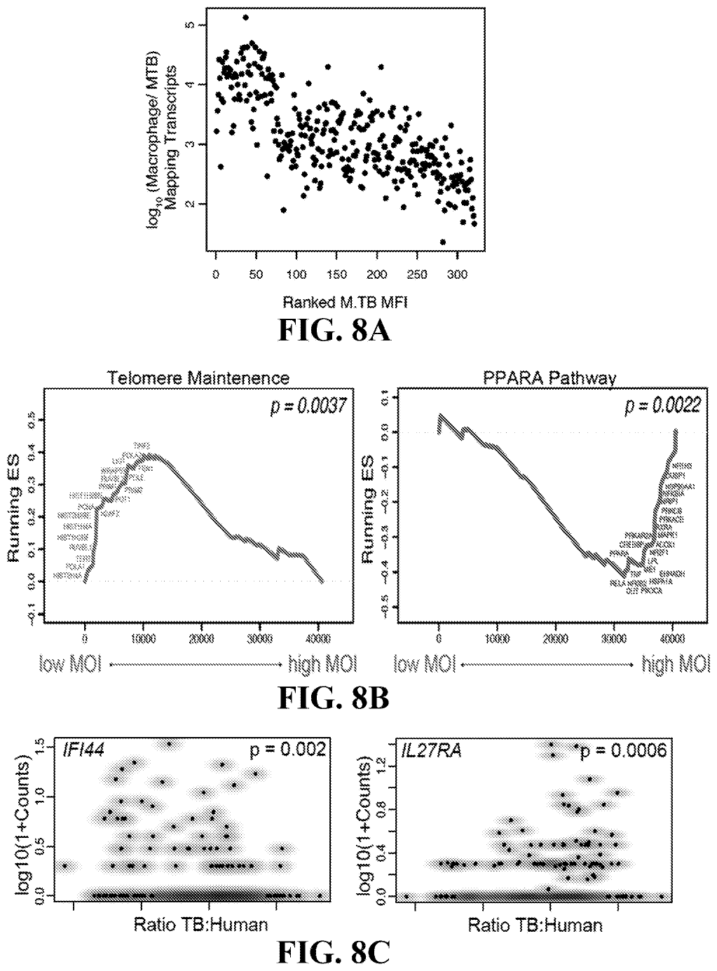

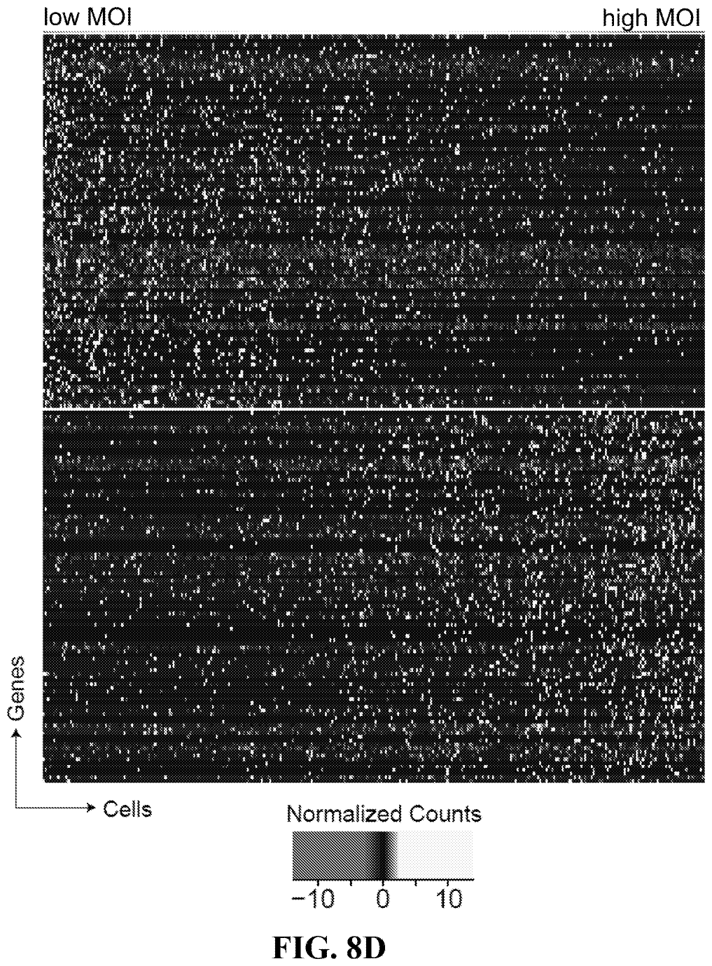

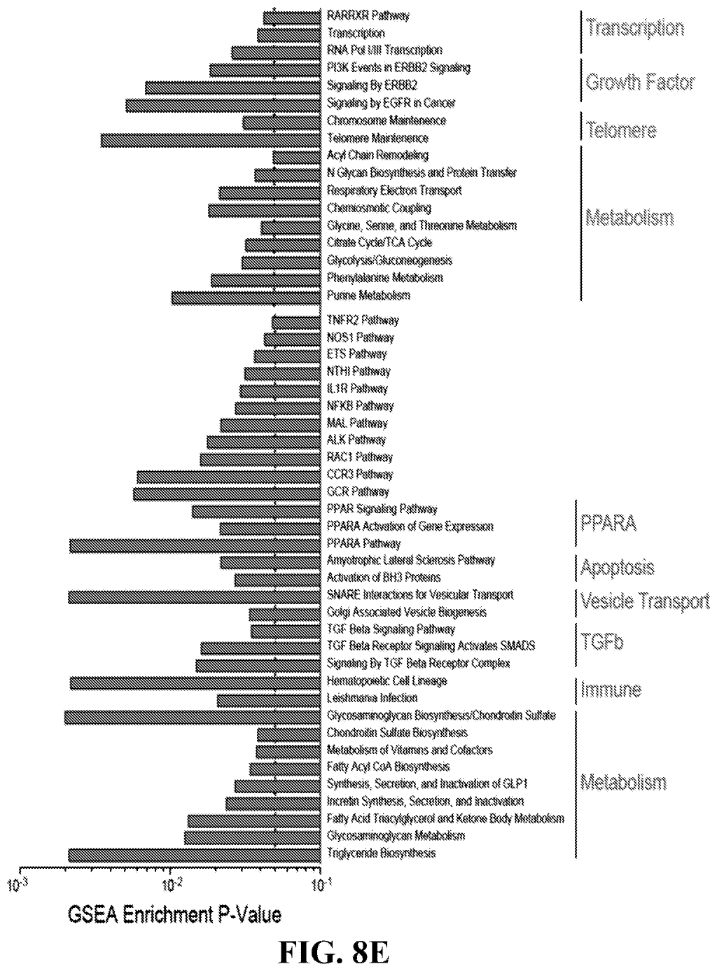

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8). Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 17)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 18)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Pharmaceuticals

Another aspect of the invention provides a composition, pharmaceutical composition or vaccine comprising the immune cells or populations thereof, as taught herein.

One aspect of the invention provides for a composition, pharmaceutical composition or vaccine directed to HIV-infected cells, including cells harbouring persistent HIV infections

One aspect of the invention provides for a composition, pharmaceutical composition or vaccine directed to MTB infected cells.

A “pharmaceutical composition” refers to a composition that usually contains an excipient, such as a pharmaceutically acceptable carrier that is conventional in the art and that is suitable for administration to cells or to a subject.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components (e.g., modulants, immunomodulants, antigens) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

For example, cells that have proliferated, or that are being differentiated in culture dishes, can be transferred onto three-dimensional solid supports in order to cause them to multiply and/or continue the differentiation process by incubating the solid support in a liquid nutrient medium of the invention, if necessary. Cells can be transferred onto a three-dimensional solid support, e.g. by impregnating the support with a liquid suspension containing the cells. The impregnated supports obtained in this way can be implanted in a human subject. Such impregnated supports can also be re-cultured by immersing them in a liquid culture medium, prior to being finally implanted. The three-dimensional solid support needs to be biocompatible so as to enable it to be implanted in a human. It may be biodegradable or non-biodegradable.

The cells or cell populations can be administered in a manner that permits them to survive, grow, propagate and/or differentiate towards desired cell types (e.g. differentiation) or cell states. The cells or cell populations may be grafted to or may migrate to and engraft within the intended organ. The terms “cell population” or “population” denote a set of cells having characteristics in common. The characteristics may include in particular the one or more marker(s) or gene or gene product signature(s) as taught herein.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified (e.g., epithelial cells, epithelial stem cells, or immune cells) and/or other active components. The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

A further aspect of the invention provides a population of the epithelial cells, epithelial stem cells, or epithelial immune cells as taught herein. The epithelial cells, epithelial stem cells, or epithelial immune cells (preferably mucosal immune cells) cells as taught herein may be comprised in a cell population. By means of example, the specified cells may constitute at least 40% (by number) of all cells of the cell population, for example, at least 45%, preferably at least 50%, at least 55%, more preferably at least 60%, at least 65%, still more preferably at least 70%, at least 75%, even more preferably at least 80%, at least 85%, and yet more preferably at least 90%, at least 95%, at least 96%, at least 97%, at least 98%, at least 99%, or even 100% of all cells of the cell population.

The isolated intestinal epithelial cells, intestinal epithelial stem cells, or intestinal immune cells (preferably intestinal epithelial cells) of populations thereof as disclosed throughout this specification may be suitably cultured or cultivated in vitro. The term “in vitro” generally denotes outside, or external to, a body, e.g., an animal or human body. The term encompasses “ex vivo”.

The terms “culturing” or “cell culture” are common in the art and broadly refer to maintenance of cells and potentially expansion (proliferation, propagation) of cells in vitro. Typically, animal cells, such as mammalian cells, such as human cells, are cultured by exposing them to (i.e., contacting them with) a suitable cell culture medium in a vessel or container adequate for the purpose (e.g., a 96-, 24-, or 6-well plate, a T-25, T-75, T-150 or T-225 flask, or a cell factory), at art-known conditions conducive to in vitro cell culture, such as temperature of 37° C., 5% v/v CO₂ and >95% humidity.

The term “medium” as used herein broadly encompasses any cell culture medium conducive to maintenance of cells, preferably conducive to proliferation of cells. Typically, the medium will be a liquid culture medium, which facilitates easy manipulation (e.g., decantation, pipetting, centrifugation, filtration, and such) thereof.

In certain example embodiments, the agent modulates HIV-infected cells by modulating one or more of the genes listed in Table 1. The genes identified in Table 1 and subsequent tables were determined using scRNA-seq analysis of a combination of healthy control, infected with HIV.

In certain example embodiments, the agent modulates HIV-infected cells by modulating one or more of the genes listed in Table 2. In another example embodiment, the agent modulates HIV-infected cells by modulating one or more of the genes listed in Table 2 (expression induced/increased in HIV+ cells) and/or Table 3 (expression suppressed/decreased in HIV+ cells). The cluster numbers in Table 2 and Table 3 refer to the clusters and cell types as labeled.

The invention having now been described by way of written description, those of skill in the art will recognize that the invention can be practiced in a variety of embodiments and that the foregoing description and examples below are for purposes of illustration and not limitation of the claims that follow.

Although the present invention and its advantages have been described in detail, it should be understood that various changes, substitutions and alterations can be made herein without departing from the spirit and scope of the invention as defined in the appended claims.

### EXAMPLES

### Example 1

HIV preferentially infects CD4 T cells, reverse transcribes its DNA, and integrates into the host genome. Infection progresses through a spike in viral load, followed by a progressive decrease in CD4⁺ T cell count. Because of the high plasma viral load, and because T cells migrate throughout different locations, virtually all tissues can be exposed to the virus, causing profound, and often irreversible changes to the adaptive and innate immune systems, and establishing a permanent pool of integrated HIV termed the “reservoir.”

Patients treated with anti-retrovirals may have undetectable virus in peripheral blood, but demonstrate HIV viral production and replication in about 1% of cells in harvested lymph nodes. Lymph nodes from suppressed donors were thawed, “reactivated/reanimated” for 18 hours with PHA/IL2 and sorted into Seq-Well arrays and evaluated for gene expression.

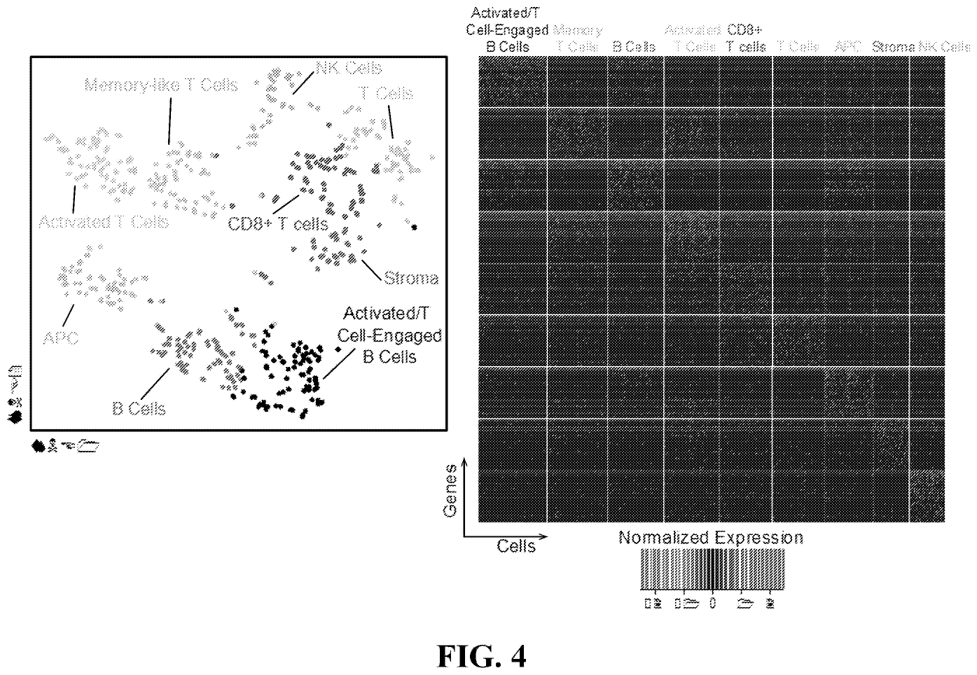

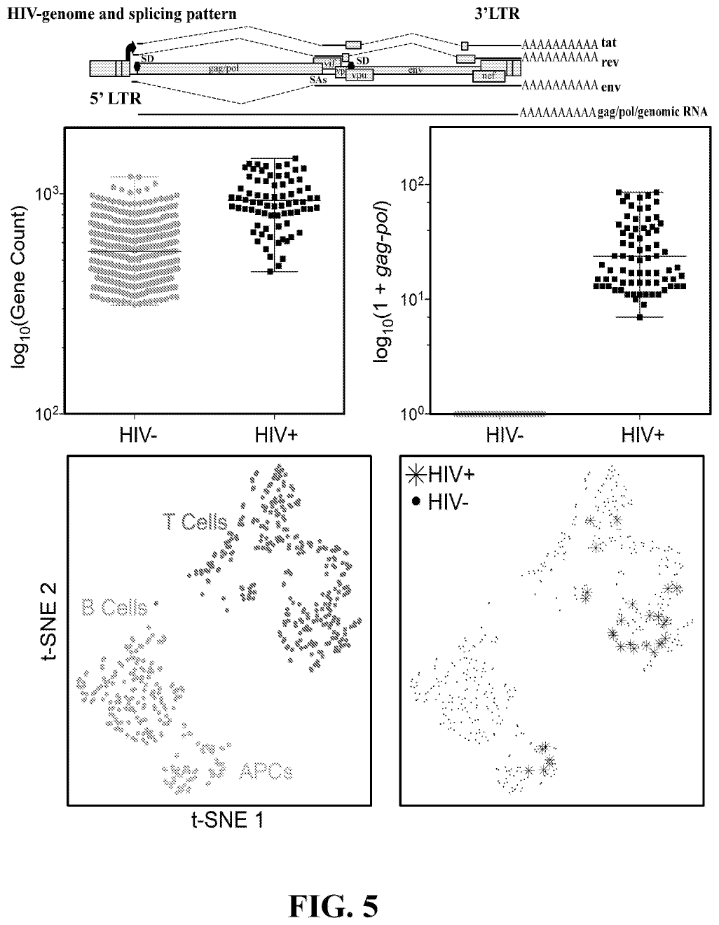

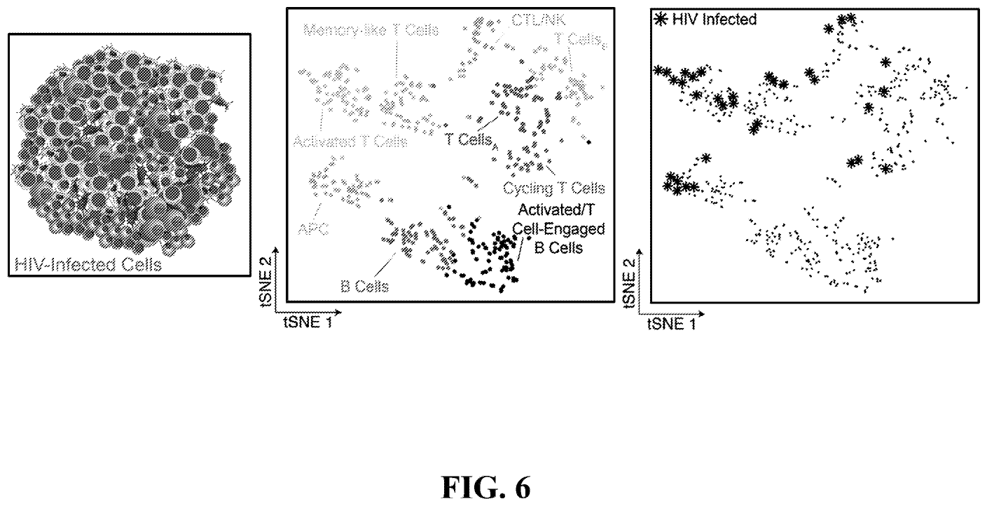

FIG. 4 provides an expression profile from lymph node from an HIV-infected, antiretroviral-treated patient. FIG. 5 shows HIV infection of subsets of T Cells and APCs. FIG. 6 shows infection status of single cells and HIV infection of subsets of T Cells and APCs.

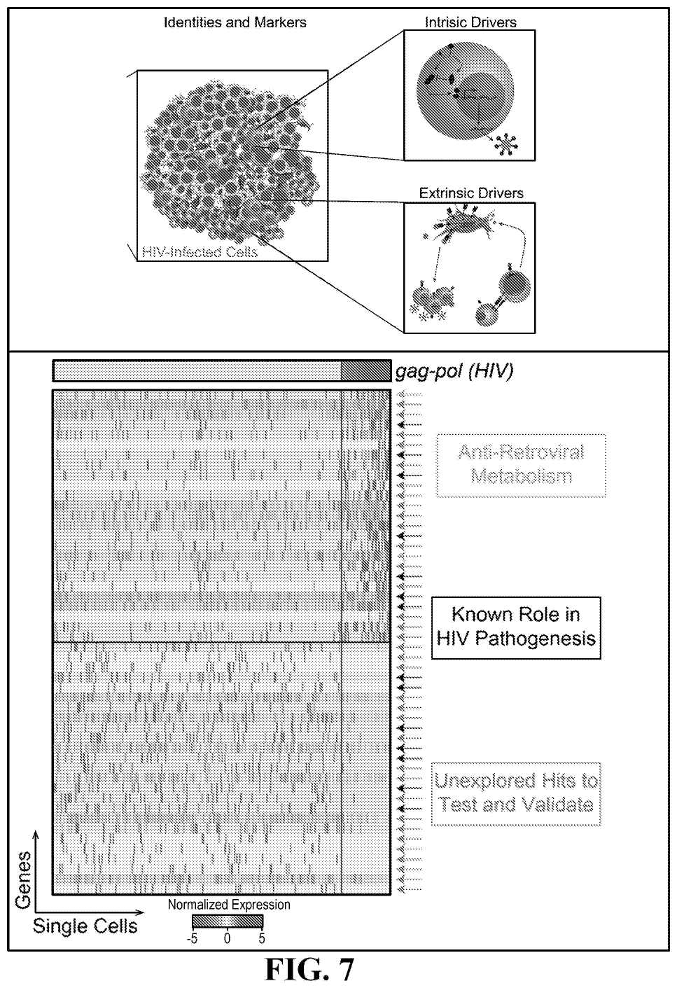

FIG. 7 demonstrates host cell gene expression in HIV infected cells of genes involved in anti-retroviral metabolism, HIV pathogenesis, as well as genes of unexplored function. The following tables provide genes differentially expressed in HIV infected cell. Approximately 16,000 genes were evaluated for differential expression between HIV⁺ and HIV⁻ cells. Table 1 identifies genes whose expression most positively correlated HIV infection. Table 2 provides a larger list of genes positively correlated with HIV infection though to a lesser extent (lower cutoff). Table 3 provides host genes most positively correlated with cell free of HIV.

TABLE 1
HIV + high cutoff

| Category | Count | Genes |
|---|---|---|
| Proteomics | 57 | TGOLN2, UTP18, CAPZA2, |
| identification | | STOML2, TCEAL8, CNOT7, |
| | | SKAP1, THADA, KLHL7, |
| | | NDUFS6, GTF2E2, WDR73, |
| | | NUDCD1, RAE1, MAPKAP1, |
| | | EIF1AY, PTBP3, BCL10, NCOA7, |
| | | TOPBP1, MESDC2, CCDC137, |
| | | ARL16, CCNC, RBBP7, MFN2, |
| | | PYCR1, DOK2, DCUN1D1, |
| | | NCOA4, ADSL, LRCH3, SNRPG, |
| | | SNX9, MEAF6, CRLF3, NOB1, |
| | | PXK, ARF5, VARS, SRRT, |
| | | CCDC124, NFAT5, STK38L, |
| | | USP33, TFDP1, PRPF40A, GPS1, |
| | | GCDH, TMEM120B, RBMX, |
| | | PTPN11, PWP1, PSMG4, MRPL28, |
| | | CUL4A, SNRNP25 |
| Acetylation | 28 | MEAF6, SNX9, CRLF3, CAPZA2, |
| | | STOML2, VARS, SRRT, NDUFS6, |
| | | GTF2E2, ATAD3B, MAPKAP1, |
| | | NFAT5, PTBP3, DDA1, STK38L, |
| | | PRPF40A, TFDP1, STX6, GCDH, |
| | | BCL10, NCOA7, RBBP7, RBMX, |
| | | PTPN11, PYCR1, DCUN1D1, |
| | | ADSL, HIGD2A |
| Phosphoprotein | 43 | TGOLN2, SNX9, MEAF6, UTP18, |
| | | CAPZA2, NOC3L, NOB1, |
| | | STOML2, VARS, SKAP1, THADA, |
| | | SRRT, GTF2E2, NUDCD1, RAE1, |
| | | CCDC124, MAPKAP1, NFAT5, |
| | | PTBP3, DDA1, STK38L, USP33, |
| | | TFDP1, PRPF40A, STX6, GPS1, |
| | | BCL10, EFCAB14, NCOA7, CCNC, |
| | | CCDC137, TOPBP1, RBBP7, |
| | | RBMX, PWP1, NOC2L, PTPN11, |
| | | MFN2, PYCR1, DOK2, CUL4A, |
| | | ADSL, LRCH3 |
| nucleoplasm | 22 | TGOLN2, GPS1, MEAF6, UTP18, |
| | | NOB1, TOPBP1, CCNC, CNOT7, |
| | | RBBP7, RBMX, NOC2L, KLHL7, |
| | | SRRT, GTF2E2, CUL4A, |
| | | MAPKAP1, NFAT5, USP33, |
| | | SNRNP25, TFDP1, SNRPG, |
| | | PRPF40A |
| Nucleus | 30 | MEAF6, UTP18, NOC3L, NOB1, |
| | | TCEAL8, CNOT7, SKAP1, KLHL7, |
| | | SRRT, GTF2E2, NUDCD1, RAE1, |
| | | MAPKAP1, NFAT5, TFDP1, |
| | | PRPF40A, GPS1, TMEM120B, |
| | | NCOA7, CCNC, TOPBP1, RBBP7, |
| | | RBMX, PTPN11, NOC2L, PWP1, |
| | | DCUN1D1, EIF5AL1, SNRNP25, |
| | | SNRPG |
| GO: 0044822~poly(A) | 12 | SRRT, GTF2E2, MRPL28, |
| RNA binding | | CCDC124, UTP18, NOC3L, PTBP3, |
| | | CCDC137, RBMX, PRPF40A, |
| | | NOC2L, SNRPG |
| GO: 0042101~T cell | 3 | BCL10, STOML2, SKAP1 |
| receptor complex | | |
| Activator | 8 | SRRT, MEAF6, NCOA4, NFAT5, |
| | | NCOA7, CCNC, RBMX, TFDP1 |
| GO: 0000398~mRNA | 5 | SRRT, RBMX, SNRNP25, |
| splicing, via spliceosome | | PRPF40A, SNRPG |
| SM00320: WD40 | 5 | WDR73, UTP18, RAE1, RBBP7, |
| | | PWP1 |
| GO: 1901796~regulation | 4 | MEAF6, TOPBP1, RBBP7, NOC2L |
| of signal transduction | | |
| by p53 class mediator | | |
| mRNA splicing | 5 | PTBP3, RBMX, SNRNP25, |
| | | PRPF40A, SNRPG |
| GO: 0005515~protein | 31 | SNX9, MEAF6, CRLF3, STOML2, |
| binding | | ARF5, VARS, CNOT7, SKAP1, |
| | | SRRT, GTF2E2, STK38L, USP33, |
| | | TFDP1, PRPF40A, STX6, BCL10, |
| | | ARL16, CCNC, TOPBP1, RBBP7, |
| | | RBMX, NOC2L, PTPN11, MFN2, |
| | | PYCR1, DCUN1D1, MRPL28, |
| | | CUL4A, HSPA13, SNRNP25, |
| | | SNRPG |
| repeat: WD 3 | 5 | WDR73, UTP18, RAE1, RBBP7, |
| | | PWP1 |
| GO: 0003723~RNA | 7 | RAE1, PTBP3, CNOT7, RBBP7, |
| binding | | RBMX, PRPF40A, SNRPG |
| WD repeat | 5 | WDR73, UTP18, RAE1, RBBP7, |
| | | PWP1 |
| repeat: WD 2 | 5 | WDR73, UTP18, RAE1, RBBP7, |
| | | PWP1 |
| repeat: WD 1 | 5 | WDR73, UTP18, RAE1, RBBP7, |
| | | PWP1 |
| IPR001680: WD40 | 5 | WDR73, UTP18, RAE1, RBBP7, |
| repeat | | PWP1 |
| IPR019775: WD40 | 4 | UTP18, RAE1, RBBP7, PWP1 |
| repeat, conserved site | | |
| mRNA processing | 5 | PTBP3, RBMX, SNRNP25, |
| | | PRPF40A, SNRPG |
| IPR017986: WD40- | 5 | WDR73, UTP18, RAE1, RBBP7, |
| repeat-containing | | PWP1 |
| domain | | |
| Ubl conjugation | 12 | MIN2, SNX9, MEAF6, CUL4A, |
| | | EIF1AY, NFAT5, ADSL, TOPBP1, |
| | | RBBP7, RBMX, USP33, PRPF40A |
| GO: 0005730~nucleolus | 8 | KLHL7, MEAF6, UTP18, RAE1, |
| | | NOC3L, CCDC137, PWP1, NOC2L |
| GO: 0043130~ubiquitin | 3 | BCL10, RAE1, USP33 |
| binding | | |
| IPR015943: WD40/Y | 5 | WDR73, UTP18, RAE1, RBBP7, |
| VTN repeat-like- | | PWP1 |
| containing domain | | |
| GO: 0015629~actin | 4 | CAPZA2, STOML2, TOPBP1, |
| cytoskeleton | | STK38L |
| Repressor | 6 | CCNC, PTBP3, CNOT7, RBBP7, |
| | | RBMX, NOC2L |
| IPR020472: G-protein | 3 | RAE1, RBBP7, PWP1 |
| beta WD-40 repeat | | |
| IPR011991: Winged | 4 | GPS1, GTF2E2, CUL4A, TFDP1 |
| helix-turn-helix | | |
| DNA-binding | | |
| domain | | |
| GO: 0005634~nucleus | 26 | UTP18, NOC3L, TCEAL8, PXK, |
| | | CNOT7, SKAP1, KLHL7, GTF2E2, |
| | | NUDCD1, RAE1, MAPKAP1, |
| | | NFAT5, PTBP3, TFDP1, BCL10, |
| | | NCOA7, TOPBP1, CCNC, RBBP7, |
| | | RBMX, PWP1, NOC2L, PTPN11, |
| | | DCUN1D1, NCOA4, SNRNP25 |
| repeat: WD 4 | 4 | UTP18, RAE1, RBBP7, PWP1 |
| mutagenesis site | 13 | TGOLN2, GCDH, BCL10, NCOA7, |
| | | PXK, SKAP1, PTPN11, MIN2, |
| | | CUL4A, NCOA4, EIF5AL1, USP33, |
| | | STK38L |
| Mitochondrion inner | 4 | NDUFS6, ATAD3B, STOML2, |
| membrane | | HIGD2A |
| Coiled coil | 16 | STX6, MEAF6, CRLF3, |
| | | TMEM120B, NOC3L, STOML2, |
| | | NCOA7, CCDC137, TCEAL8, |
| | | ARF5, VARS, THADA, MIN2, |
| | | SRRT, ATAD3B, CCDC124 |
| GO: 0071004~U2- | 2 | PRPF40A, SNRPG |
| type prespliceosome | | |
| GO: 0045944-positive | 8 | CRLF3, NFAT5, NCOA7, CCNC, |
| regulation of | | CNOT7, SKAP1, RBMX, TFDP1 |
| transcription from | | |
| RNA polymerase II | | |
| promoter | | |
| GO: 0005737~cytoplasm | 25 | GPS1, BCL10, SNX9, MEAF6, |
| | | CRLF3, TOPBP1, PXK, ARF5, |
| | | SKAP1, PTPN11, NOC2L, KLHL7, |
| | | SRRT, GTF2E2, NUDCD1, |
| | | MRPL28, CCDC124, RAE1, |
| | | MAPKAP1, NFAT5, LRCH3, |
| | | USP33, STK38L, SNRNP25, |
| | | PRPF40A |
| GO: 0005685~U1 snRNP | 2 | PRPF40A, SNRPG |
| GO: 0070469~respiratory | 2 | NDUFS6, HIGD2A |
| chain | | |
| Spliceosome | 3 | RBMX, SNRNP25, SNRPG |
| GO: | 5 | NDUFS6, ATAD3B, MRPL28, |
| 0005743~mitochondrial | | STOML2, HIGD2A |
| inner membrane | | |
| Mitochondrion | 8 | MFN2, GCDH, PYCR1, NDUFS6, |
| | | ATAD3B, MRPL28, STOML2, |
| | | HIGD2A |
| Isopeptide bond | 8 | MEAF6, CUL4A, EIF1AY, NFAT5, |
| | | ADSL, RBBP7, RBMX, PRPF40A |
| hsa03040: Spliceosome | 3 | RBMX, PRPF40A, SNRPG |
| GO: 0031625~ubiquitin | 4 | MFN2, BCL10, SNX9, CUL4A |
| protein ligase binding | | |
| GO: 0005794~Golgi | 7 | TGOLN2, STX6, MAPKAP1, |
| apparatus | | TOPBP1, ARF5, USP33, PWP1 |
| GO: 0005802~trans- | 3 | TGOLN2, STX6, SNX9 |
| Golgi network | | |
| Transcription | 13 | SRRT, MEAF6, GTF2E2, NCOA4, |
| | | NFAT5, NCOA7, CCNC, TCEAL8, |
| | | CNOT7, RBBP7, RBMX, NOC2L, |
| | | TFDP1 |
| GO: 0000715~nucleotide- | 2 | GPS1, CUL4A |
| excision repair, DNA | | |
| damage recognition | | |
| GO: 0046580~negative | 2 | MFN2, MAPKAP1 |
| regulation of Ras protein | | |
| signal transduction | | |
| GO: 0005689~U12-type | 2 | SNRNP25, SNRPG |
| spliceosomal complex | | |
| Protein biosynthesis | 3 | EIF5AL1, EIF1AY, VARS |
| Chromosomal | 4 | BCL10, MEAF6, NCOA4, THADA |
| rearrangement | | |
| GO: 0050852~T cell | 3 | BCL10, STOML2, SKAP1 |
| receptor signaling | | |
| pathway | | |

HIV + low cutoff

| | | |
|---|---|---|
| Category | Count | Genes |

Enrichment Score: 5.645742662204743

| | | |
|---|---|---|
| Mitochondrion | 43 | HSD17B10, MRPS35, OXA1L, |
| | | NDUFB6, MRPS33, COA3, |
| | | FKBP4, TIMM10, STOML2, |
| | | PTRH2, MTIF3, HADHA, |
| | | NDUFS6, MRPL13, ATAD3B, |
| | | DDX3X, TIMM9, REXO2, |
| | | MRPL54, ABHD10, YRDC, |
| | | APEX1, GCDH, MRPS26, |
| | | MRPL4, NDUFA2, MMADHC, |
| | | HCLS1, AK2, TOMM40, |
| | | TMEM126B, SOD2, MFN2, |
| | | PYCR1, MRPL22, MRPL28, |
| | | PPM1K, CLPP, ATP5C1, |
| | | MRPL48, SLC25A39, BCO2, |
| | | HIGD2A |
| GO: | 24 | MRPS35, MRPS26, MRPL4, |
| 0005743~mitochondrial | | NDUFA2, OXA1L, MRPS33, |
| inner membrane | | NDUFB6, TIMM10, STOML2, |
| | | AK2, TMEM126B, HADHA, |
| | | SOD2, NDUFS6, MRPL22, |
| | | MRPL13, ATAD3B, MRPL28, |
| | | TIMM9, MRPL54, ATP5C1, |
| | | MRPL48, SLC25A39, HIGD2A |
| transit peptide: | 20 | GCDH, MRPS35, MRPS26, |
| Mitochondrion | | OXA1L, MMADHC, PTRH2, |
| | | MTIF3, HADHA, SOD2, |
| | | NDUFS6, MRPL22, MRPL28, |
| | | PPM1K, CLPP, REXO2, |
| | | MRPL54, ATP5C1, ABHD10, |
| | | YRDC, MRPL48 |
| Transit peptide | 21 | GCDH, MRPS35, MRPS26, |
| | | OXA1L, MMADHC, STOML2, |
| | | PTRH2, MTIF3, HADHA, SOD2, |
| | | NDUFS6, MRPL22, MRPL28, |
| | | PPM1K, REXO2, CLPP, |
| | | MRPL54, ATP5C1, ABHD10, |
| | | YRDC, MRPL48 |

Enrichment Score: 4.175905846577495

| | | |
|---|---|---|
| mRNA splicing | 20 | HNRNPA1L2, SRSF1, DDX39A, |
| | | PRPF4B, YTHDC1, PRPF39, |
| | | RBMX, GCFC2, PRPF6, CIR1, |
| | | CD2BP2, CDC40, DHX15, |
| | | RBMXL1, PTBP3, SREK1IP1, |
| | | SNRNP25, THOC1, SNRPG, |
| | | PRPF40A |
| mRNA processing | 21 | HNRNPA1L2, SRSF1, DDX39A, |
| | | PRPF4B, YTHDC1, PRPF39, |
| | | RBMX, GCFC2, SLBP, PRPF6, |
| | | CIR1, CD2BP2, CDC40, DHX15, |
| | | RBMXL1, PTBP3, SREK1IP1, |
| | | SNRNP25, THOC1, SNRPG, |
| | | PRPF40A |
| GO: 0000398~mRNA | 15 | SRSF1, DDX39A, PRPF4B, |
| splicing, via spliceosome | | POLR2K, CWC27, RBMX, |
| | | PRPF6, SRRT, CD2BP2, |
| | | HTATSF1, CDC40, DHX15, |
| | | SNRNP25, SNRPG, PRPF40A |
| GO: 0008380~RNA | 12 | HNRNPA1L2, CIR1, PRPF4B, |
| splicing | | CDC40, DHX15, RBMXL1, |
| | | PTBP3, SREK1IP1, SNRNP25, |
| | | THOC1, PRPF6, SNRPG |
| hsa03040: Spliceosome | 10 | HNRNPA1L2, SRSF1, CDC40, |
| | | DHX15, RBMXL1, RBMX, |
| | | THOC1, PRPF6, PRPF40A, |
| | | SNRPG |
| Spliceosome | 8 | HNRNPA1L2, SRSF1, PRPF4B, |
| | | CDC40, RBMX, SNRNP25, |
| | | PRPF6, SNRPG |
| GO: 0071013~catalytic | 7 | SRSF1, PRPF4B, CWC27, |
| step 2 spliceosome | | CDC40, RBMX, PRPF6, SNRPG |
| GO: | 5 | HNRNPA1L2, DDX39A, CDC40, |
| 0005681~spliceosomal | | PRPF6, SNRPG |
| complex | | |

Enrichment Score: 3.1886930162792817

| | | |
|---|---|---|
| Protein biosynthesis | 16 | AARS, DENR, VARS, ETF1, |
| | | MTIF3, EIF2S1, EIF5AL1, EIF3F, |
| | | EIF1AY, HARS, TCEB3, TCEA1, |
| | | EIF1, SUPT5H, MCTS1, EIF4E2 |
| Initiation factor | 8 | EIF2S1, EIF1AY, EIF3F, EIF1, |
| | | DENR, MCTS1, MTIF3, EIF4E2 |
| GO: 0003743~translation | 8 | EIF2S1, EIF1AY, EIF3F, EIF1, |
| initiation factor activity | | DENR, MCTS1, MTIF3, EIF4E2 |
| GO: 0032790~ribosome | 3 | DENR, MCTS1, MTIF3 |
| disassembly | | |
| hsa03013: RNA transport | 10 | NXT1, NUP62, RAE1, EIF2S1, |
| | | EIF1AY, PABPC4, EIF3F, EIF1, |
| | | EIF4E2, THOC1 |
| GO: | 7 | EIF2S1, EIF1AY, EIF3F, RPL35, |
| 0006413~translational | | EIF1, RPL39, EIF4E2 |
| initiation | | |
| GO: 0001731~formation | 3 | EIF3F, DENR, MCTS1 |
| of translation preinitiation | | |
| complex | | |
| GO: 0008135~translation | 3 | EIF1, MTIF3, EIF4E2 |
| factor activity, RNA | | |
| binding | | |

Enrichment Score: 3.1727290299421798

| | | |
|---|---|---|
| Ribonucleoprotein | 18 | HNRNPA1L2, MRPS35, |
| | | MRPS26, MRPL4, MRPS33, |
| | | RPL35, RPL39, SRP19, RBMX, |
| | | RPS19BP1, SLBP, MRPL22, |
| | | MRPL13, MRPL28, MRPL54, |
| | | RBMXL1, MRPL48, SNRPG |
| GO: | 9 | MRPS35, MRPS26, MRPL22, |
| 0070125~mitochondrial | | MRPL4, MRPL13, MRPS33, |
| translational elongation | | MRPL28, MRPL54, MRPL48 |
| GO: | 9 | MRPS35, MRPS26, MRPL22, |
| 0070126~mitochondrial | | MRPL4, MRPL13, MRPS33, |
| translational termination | | MRPL28, MRPL54, MRPL48 |
| Ribosomal protein | 12 | MRPS35, MRPS26, MRPL22, |
| | | MRPL4, MRPL13, MRPS33, |
| | | MRPL28, MRPL54, RPL35, |
| | | MRPL48, RPL39, RPS19BP1 |
| GO: 0006412~translation | 10 | MRPL22, MRPL4, MRPL13, |
| | | MRPS33, MRPL28, PABPC4, |
| | | HARS, RPL35, SLC25A39, |
| | | RPL39 |
| GO: 0003735~structural | 9 | MRPS35, MRPL22, MRPL4, |
| constituent of ribosome | | MRPL13, MRPS33, MRPL28, |
| | | RPL35, SLC25A39, RPL39 |
| hsa03010: Ribosome | 6 | MRPL22, MRPL4, MRPL13, |
| | | MRPL28, RPL35, RPL39 |

Enrichment Score: 2.5160173816834086

| | | |
|---|---|---|
| GO: 0006406~mRNA | 9 | NXT1, SRSF1, DDX39A, NUP62, |
| export from nucleus | | RAE1, CDC40, SMG1, SLBP, |
| | | THOC1 |
| GO: 0006405~RNA | 6 | NXT1, SRSF1, DDX39A, NUP62, |
| export from nucleus | | CDC40, THOC1 |
| GO: 0006369~termination | 6 | SRSF1, DDX39A, CDC40, SLBP, |
| of RNA polymerase II | | THOC1, SNRPG |
| transcription | | |
| GO: 0031124~mRNA | 4 | SRSF1, DDX39A, CDC40, |
| 3′-end processing | | THOC1 |

Enrichment Score: 2.126966020626473

| | | |
|---|---|---|
| GO: | 7 | TAF11, ADRM1, GTF2E2, |
| 0006368~transcription | | POLR2K, TCEB3, TCEA1, |
| elongation from RNA | | SUPT5H |
| polymerase II promoter | | |
| Elongation factor | 4 | EIF5AL1, TCEB3, TCEA1, |
| | | SUPT5H |
| GO: 0003746~translation | 4 | EIF5AL1, TCEB3, TCEA1, |
| elongation factor activity | | SUPT5H |

Enrichment Score: 1.874526987901123

| | | |
|---|---|---|
| DNA repair | 12 | UBE2N, PSMD14, CUL4A, |
| | | BABAM1, SMG1, PRKDC, |
| | | TOPBP1, USP10, APEX1, SMC3, |
| | | TRIP12, BOD1L1 |
| DNA damage | 13 | PRKDC, SMG1, TOPBP1, SMC3, |
| | | BOD1L1, UBE2N, PSMD14, |
| | | CUL4A, BABAM1, USP10, |
| | | APEX1, MCTS1, TRIP12 |
| GO: 0006281~DNA repair | 7 | SMG1, TOPBP1, APEX1, |
| | | ASF1A, SMC3, TRIP12, BOD1L1 |

Enrichment Score: 1.8713442584451319

| | | |
|---|---|---|
| Neuropathy | 7 | MFN2, AARS, LMNA, HARS, |
| | | WNK1, DNMT1, DNM2 |
| Charcot-Marie-Tooth | 5 | MFN2, AARS, LMNA, HARS, |
| disease | | DNM2 |
| Neurodegenemtion | 7 | MFN2, ELOVL5, AARS, LMNA, |
| | | HARS, WNK1, DNM2 |

Enrichment Score: 1.8468573086564095

| | | |
|---|---|---|
| Cell division | 13 | SNX9, ATAD3B, GNAI2, |
| | | CCDC124, IST1, CDC40, CKS2, |
| | | CENPV, BABAM1, SMC2, |
| | | SMC3, MCM5, PRPF40A |
| Cell cycle | 18 | SNX9, USP8, GNAI2, SMC2, |
| | | MCM4, SMC3, MCM5, |
| | | ATAD3B, CSNK2A1, CCDC124, |
| | | IST1, CDC40, BABAM1, |
| | | CENPV, CKS2, MCTS1, TFDP1, |
| | | PRPF40A |
| GO: 0051301~cell | 12 | ATAD3B, GNAI2, CCDC124, |
| | | IST1, CDC40, CKS2, CENPV, |
| | | BABAM1, SMC2, MCM5, SMC3, |
| division | | PRPF40A |

Enrichment Score: 1.679241294734509

| | | |
|---|---|---|
| Isomerase | 7 | TOP1, FKBP4, CWC27, PPID, |
| | | FKBP3, TOPBP1, TSTA3 |
| Rotamase | 4 | FKBP4, CWC27, PPID, FKBP3 |
| GO: 0061077~chaperone- | 4 | CSNK2A1, FKBP4, PPID, FKBP3 |
| mediated protein folding | | |
| GO: 0000413~protein | 4 | FKBP4, CWC27, PPID, FKBP3 |
| peptidyl-prolyl | | |
| isomerization | | |
| GO: 0003755~peptidyl- | 4 | FKBP4, CWC27, PPID, FKBP3 |
| prolyl cis-trans isomerase | | |
| activity | | |

Enrichment Score: 1.6747845841936386

| | | |
|---|---|---|
| Repressor | 20 | RCOR1, CCNC, NFKB2, MAF1, |
| | | CNOT7, RBBP7, RBMX, GCFC2, |
| | | NOC2L, SUZ12, KDM1A, CIR1, |
| | | SP3, MLX, DNMT1, PTBP3, |
| | | SUPT5H, APEX1, C1D, KAT6A |
| Activator | 19 | MEAF6, FOXO1, NCOA7, |
| | | PHF11, CCNC, NFKB2, RBMX, |
| | | PURA, SRRT, NCOA4, SP3, |
| | | HTATSF1, MLX, NFAT5, |
| | | DNMT1, SUPT5H, APEX1, |
| | | KAT6A, TFDP1 |
| Transcription | 46 | MEAF6, POLR2K, FOXO1, |
| | | TCEAL8, NFKB2, MAF1, |
| | | CNOT7, VPS72, PRIM1, SRRT, |
| | | KDM1A, GTF2E2, CSNK2A1, |
| | | CIR1, DDX3X, HTATSF1, |
| | | GTF3C6, NFAT5, TCEA1, |
| | | SUPT5H, ASF1A, APEX1, |
| | | TFDP1, CHD3, RCOR1, NCOA7, |
| | | POLR1C, CCNC, PHF11, RBBP7, |
| | | UBE2L3, RBMX, GCFC2, PURA, |
| | | NOC2L, TAF11, SUZ12, NCOA4, |
| | | MLX, SP3, TCEB3, DNMT1, |
| | | MCTS1, THOC1, KAT6A, C1D |
| Transcription regulation | 41 | MEAF6, FOXO1, TCEAL8, |
| | | NFKB2, CNOT7, MAF1, VPS72, |
| | | SRRT, KDM1A, GTF2E2, |
| | | CSNK2A1, CIR1, DDX3X, |
| | | HTATSF1, NFAT5, TCEA1, |
| | | SUPT5H, ASF1A, APEX1, |
| | | TFDP1, CHD3, RCOR1, NCOA7, |
| | | CCNC, PHF11, RBBP7, UBE2L3, |
| | | GCFC2, PURA, NOC2L, TAF11, |
| | | SUZ12, NCOA4, MLX, SP3, |
| | | TCEB3, DNMT1, MCTS1, |
| | | THOC1, KAT6A, C1D |
| GO: | 38 | MEAF6, POLR2K, FOXO1, |
| 0006351~transcription, | | TCEAL8, CNOT7, MAF1, |
| DNA-templated | | VPS72, KDM1A, CIR1, |
| | | CSNK2A1, DDX3X, HTATSF1, |
| | | GTF3C6, TCEA1, ASF1A, |
| | | APEX1, TFDP1, CHD3, RCOR1, |
| | | NCOA7, POLR1C, PHF11, |
| | | RBBP7, UBE2L3, GCFC2, |
| | | PURA, PWP1, NOC2L, SUZ12, |
| | | NUP62, NCOA4, MLX, SP3, |
| | | DNMT1, TCEB3, MCTS1, |
| | | KAT6A, C1D |

Enrichment Score: 1.6402823990813864

| | | |
|---|---|---|
| Nucleotide-binding | 40 | PRPF4B, GNAI2, DTYMK, |
| | | CTPS1, RAB1B, UBA6, PRKDC, |
| | | ASNS, ARF5, VARS, HPRT1, |
| | | ATAD3B, CSNK2A1, DDX3X, |
| | | DHX15, STK38L, CHD3, |
| | | DDX39A, ITK, AARS, ATP11B, |
| | | WNK1, AK2, SMG1, ACLY, |
| | | ARL16, UBE2L3, MCM4, SMC2, |
| | | MCM5, SMC3, MFN2, UBE2N, |
| | | HYOU1, PSMC4, RFK, ARF3, |
| | | HARS, HSPA13, DNM2 |
| ATP-binding | 32 | PRPF4B, DTYMK, CTPS1, |
| | | UBA6, PRKDC, ASNS, VARS, |
| | | ATAD3B, CSNK2A1, DDX3X, |
| | | DHX15, STK38L, CHD3, |
| | | DDX39A, ITK, AARS, ATP11B, |
| | | WNK1, SMG1, AK2, ACLY, |
| | | UBE2L3, SMC2, MCM4, MCM5, |
| | | SMC3, UBE2N, HYOU1, PSMC4, |
| | | RFK, HARS, HSPA13 |
| GO: 0005524~ATP | 35 | PRPF4B, FKBP4, DTYMK, |
| binding | | CTPS1, UBA6, PRKDC, ASNS, |
| | | PXK, VARS, ATAD3B, |
| | | CSNK2A1, DDX3X, DHX15, |
| | | STK38L, CHD3, DDX39A, ITK, |
| | | SMCHD1, AARS, ATP11B, |
| | | WNK1, SMG1, AK2, ACLY, |
| | | UBE2L3, SMC2, MCM4, MCM5, |
| | | SMC3, UBE2N, HYOU1, |
| | | PSMC4, RFK, HARS, HSPA13 |
| nucleotide phosphate- | 19 | DDX39A, ITK, PRPF4B, |
| binding region: ATP | | DTYMK, WNK1, UBA6, AK2, |
| | | ACLY, SMC2, MCM4, SMC3, |
| | | MCM5, ATAD3B, CSNK2A1, |
| | | DDX3X, PSMC4, DHX15, |
| | | STK38L, CHD3 |

Enrichment Score: 1.5505018585302555

| | | |
|---|---|---|
| GO: 0006890~retrograde | 7 | COPB2, KDELR2, ARF3, |
| vesicle-mediated transport, | | TMED10, RAB1B, LMAN2, |
| Golgi to ER | | ARF5 |
| GO: 0030133~transport | 6 | TGOLN2, COPB2, KDELR2, |
| vesicle | | ERP29, TMED10, RAB1B |
| GO: 0033116~endoplasmic | 4 | TMED10, RAB1B, LMAN2, |
| reticulum-Golgi | | ERGIC3 |
| intermediate compartment | | |
| membrane | | |
| GO: 0006888~ER to Golgi | 5 | HYOU1, COPB2, TMED10, |
| vesicle-mediated transport | | RAB1B, LMAN2 |

Enrichment Score: 1.3417297598441402

| | | |
|---|---|---|
| GO: 0006270~DNA | 5 | PRIM1, TOPBP1, MCM4, |
| replication initiation | | MCM5, PURA |
| hsa03030: DNA replication | 4 | PRIM1, POLE4, MCM4, MCM5 |
| GO: 0000082~G1/S | 5 | PRIM1, CUL4A, CRLF3, MCM4, |
| transition of mitotic cell | | MCM5 |
| cycle | | |
| DNA replication | 4 | PRIM1, RBBP7, MCM4, MCM5 |
| GO: 0006260~DNA | 5 | TOP1, TOPBP1, RBBP7, MCM4, |
| replication | | MCM5 |

Enrichment Score: 1.312801362788877

| | | |
|---|---|---|
| hsa00240: Pyrimidine | 7 | PRIM1, POLE4, POLR2K, |
| metabolism | | DTYMK, CTPS1, POLR1C, |
| | | ENTPD4 |
| hsa00230: Purine | 8 | PRIM1, POLE4, POLR2K, ADSL, |
| metabolism | | AK2, POLR1C, ENTPD4, HPRT1 |
| DNA-directed RNA | 3 | PRIM1, POLR2K, POLR1C |
| polymerase | | |

Enrichment Score: 1.3089793413612678

| | | |
|---|---|---|
| SM00320: WD40 | 10 | COPB2, WDR36, WDR73, |
| | | UTP18, RAE1, CDC40, AAMP, |
| | | WDR4, RBBP7, PWP1 |
| repeat: WD 3 | 10 | COPB2, WDR36, WDR73, |
| | | UTP18, RAE1, CDC40, AAMP, |
| | | WDR4, RBBP7, PWP1 |
| WD repeat | 10 | COPB2, WDR36, WDR73, |
| | | UTP18, RAE1, CDC40, AAMP, |
| | | WDR4, RBBP7, PWP1 |
| repeat: WD 1 | 10 | COPB2, WDR36, WDR73, |
| | | UTP18, RAE1, CDC40, AAMP, |
| | | WDR4, RBBP7, PWP1 |
| repeat: WD 2 | 10 | COPB2, WDR36, WDR73, |
| | | UTP18, RAE1, CDC40, AAMP, |
| | | WDR4, RBBP7, PWP1 |
| IPR001680: WD40 | 10 | COPB2, WDR36, WDR73, |
| repeat | | UTP18, RAE1, CDC40, AAMP, |
| | | WDR4, RBBP7, PWP1 |
| repeat: WD 4 | 9 | COPB2, WDR36, UTP18, RAE1, |
| | | CDC40, AAMP, WDR4, RBBP7, |
| | | PWP1 |
| IPR019775: WD40 | 7 | WDR36, UTP18, RAE1, CDC40, |
| repeat, conserved | | AAMP, RBBP7, PWP1 |
| site | | |
| IPR017986: WD40- | 10 | COPB2, WDR36, WDR73, |
| repeat-containing | | UTP18, RAE1, CDC40, AAMP, |
| domain | | WDR4, RBBP7, PWP1 |
| IPR015943: WD40/ | 10 | COPB2, WDR36, WDR73, |
| YVTN repeat-like- | | UTP18, RAE1, CDC40, AAMP, |
| containing domain | | WDR4, RBBP7, PWP1 |
| repeat: WD 5 | 7 | COPB2, WDR36, UTP18, CDC40, |
| | | AAMP, RBBP7, PWP1 |
| repeat: WD 6 | 6 | COPB2, WDR36, UTP18, CDC40, |
| | | AAMP, RBBP7 |
| IPR020472: G- | 4 | COPB2, RAE1, RBBP7, PWP1 |
| protein beta WD-40 | | |
| repeat | | |
| repeat: WD 7 | 5 | COPB2, WDR36, CDC40, AAMP, |
| | | RBBP7 |
| repeat: WD 8 | 3 | COPB2, WDR36, AAMP |

Enrichment Score: 1.2247677909485253

| | | |
|---|---|---|
| SM00360: RRM | 8 | HNRNPA1L2, SRSF1, HTATSF1, |
| | | PABPC4, RBMXL1, RBM6, |
| | | PTBP3, RBMX |
| SM00361: RRM_1 | 3 | PABPC4, RBMXL1, RBMX |
| IPR012677: Nucleotide- | 9 | HNRNPA1L2, SRSF1, SRRT, |
| binding, alpha-beta plait | | HTATSF1, PABPC4, RBMXL1, |
| | | RBM6, PTBP3, RBMX |
| GO: 0000166~nucleotide | 11 | HNRNPA1L2, SRSF1, SRRT, |
| binding | | HTATSF1, PABPC4, RBMXL1, |
| | | RBM6, PTBP3, PXK, HPRT1, |
| | | RBMX |
| IPR003954: RNA | 3 | PABPC4, RBMXL1, RBMX |
| recognition motif | | |
| domain, eukaryote | | |
| IPR000504: RNA | 8 | HNRNPA1L2, SRSF1, HTATSF1, |
| recognition motif domain | | PABPC4, RBMXL1, RBM6, |
| | | PTBP3, RBMX |
| GO: 0030529~intracellular | 6 | HNRNPA1L2, NUP62, PABPC4, |
| ribonucleoprotein | | RBMXL1, RBMX, SLBP |
| complex | | |
| domain: RRM 1 | 5 | HNRNPA1L2, SRSF1, HTATSF1, |
| | | PABPC4, PTBP3 |
| domain: RRM 2 | 5 | HNRNPA1L2, SRSF1, HTATSF1, |
| | | PABPC4, PTBP3 |

Enrichment Score: 1.1799339425906339

| | | |
|---|---|---|
| GO: 0050852~T cell | 9 | UBE2N, ITK, BCL10, PSMB4, |
| receptor signaling | | PSMD14, PSMB7, PSMC4, |
| pathway | | STOML2, SKAP1 |
| GO: 0038061~NIK/ | 6 | PSMB4, PSMD14, PSMB7, |
| NF-kappaB | | PSMC4, NFKB2, PPP4C |
| signaling | | |
| Proteasome | 5 | PSMB4, ADRM1, PSMD14, |
| | | PSMB7, PSMC4 |
| GO: 0000502~proteasome | 5 | PSMB4, ADRM1, PSMD14, |
| complex | | PSMB7, PSMC4 |
| GO: 0038095~Fc-epsilon | 8 | UBE2N, ITK, BCL10, PSMB4, |
| receptor signaling | | PSMD14, PSMB7, PSMC4, |
| pathway | | PPP3R1 |
| GO: 0002223~stimulatory | 6 | UBE2N, BCL10, PSMB4, |
| C-type lectin receptor | | PSMD14, PSMB7, PSMC4 |
| signaling pathway | | |
| GO: 0006521~regulation | 4 | PSMB4, PSMD14, PSMB7, |
| of cellular amino acid | | PSMC4 |
| metabolic process | | |
| hsa03050: Proteasome | 4 | PSMB4, PSMD14, PSMB7, |
| | | PSMC4 |
| GO: 0000209~protein | 7 | PSMB4, PSMD14, PSMB7, |
| polyubiquitination | | PSMC4, UBE2V2, UBE2L3, |
| | | TRIP12 |
| GO: 0002479~antigen | 4 | PSMB4, PSMD14, PSMB7, |
| processing and presentation | | PSMC4 |
| of exogenous peptide | | |
| antigen via MHC class I, | | |
| TAP-dependent | | |
| GO: 0043488~regulation | 5 | PSMB4, PSMD14, PSMB7, |
| of mRNA stability | | PSMC4, APEX1 |
| GO: 0051436~negative | 4 | PSMB4, PSMD14, PSMB7, |
| regulation of ubiquitin- | | PSMC4 |
| protein ligase activity | | |
| involved in mitotic cell | | |
| cycle | | |
| GO: 0051437~positive | 4 | PSMB4, PSMD14, PSMB7, |
| regulation of ubiquitin- | | PSMC4 |
| protein ligase activity | | |
| involved in regulation | | |
| of mitotic cell cycle | | |
| transition | | |
| GO: 0031145~anaphase- | 4 | PSMB4, PSMD14, PSMB7, |
| promoting complex- | | PSMC4 |
| dependent catabolic | | |
| process | | |
| GO: 0060071~Wnt | 4 | PSMB4, PSMD14, PSMB7, |
| signaling pathway, planar | | PSMC4 |
| cell polarity pathway | | |
| GO: 0090090~negative | 5 | PSMB4, PSMD14, PSMB7, |
| regulation of canonical | | PSMC4, FOXO1 |
| Wnt signaling pathway | | |
| GO: 0033209~tumor | 4 | PSMB4, PSMD14, PSMB7, |
| necrosis factor-mediated | | PSMC4 |
| signaling pathway | | |
| GO: 0090263~positive | 4 | PSMB4, PSMD14, PSMB7, |
| regulation of canonical | | PSMC4 |
| Wnt signaling pathway | | |
| GO: 0043161~proteasome- | 5 | PSMB4, PSMD14, PSMB7, |
| mediated ubiquitin- | | CUL4A, PSMC4 |
| dependent protein catabolic | | |
| process | | |
| GO: 0000165~MAPK | 5 | PSMB4, PSMD14, PSMB7, |
| cascade | | PSMC4, CCL5 |

Enrichment Score: 0.9835956335357907

| | | |
|---|---|---|
| GO: 0005643~nuclear | 5 | NXT1, NUP62, RAE1, EIF5AL1, |
| pore | | KPNA3 |
| GO: 0075733~intracellular | 3 | NUP62, RAE1, KPNA3 |
| transport of virus | | |
| GO: 0006606~protein | 3 | NUP62, RAE1, KPNA3 |
| import into nucleus | | |

Enrichment Score: 0.9465955034396142

| | | |
|---|---|---|
| GO: 0032981~mitochondrial | 5 | NDUFS6, NDUFA2, OXA1L, |
| respiratory chain complex I | | NDUFB6, TMEM126B |
| assembly | | |
| hsa05010: Alzheimer's | 8 | HSD17B10, NDUFS6, NDUFA2, |
| disease | | CASP3, NDUFB6, PPP3R1, |
| | | ATP5C1, ITPR3 |
| Respiratory chain | 4 | NDUFS6, NDUFA2, NDUFB6, |
| | | HIGD2A |
| hsa05012: Parkinson's | 7 | NDUFS6, NDUFA2, CASP3, |
| disease | | NDUFB6, GNAI2, ATP5C1, |
| | | UBE2L3 |
| hsa05016: Huntington's | 8 | NDUFS6, NDUFA2, CASP3, |
| disease | | NDUFB6, POLR2K, RCOR1, |
| | | ATP5C1, SOD2 |
| GO: 0005747~mitochondrial | 3 | NDUFS6, NDUFA2, NDUFB6 |
| respiratory chain complex I | | |
| hsa04932: Non-alcoholic | 6 | NDUFS6, NDUFA2, CASP3, |
| fatty liver disease (NAFLD) | | NDUFB6, EIF2S1, MLX |
| GO: 0008137~NADH | 3 | NDUFS6, NDUFA2, NDUFB6 |
| dehydrogenase (ubiquinone) | | |
| activity | | |
| GO: 0006120~mitochondrial | 3 | NDUFS6, NDUFA2, NDUFB6 |
| electron transport, NADH to | | |
| ubiquinone | | |
| Electron transport | 4 | NDUFS6, NDUFA2, NDUFB6, |
| | | HIGD2A |
| hsa00190: Oxidative | 4 | NDUFS6, NDUFA2, NDUFB6, |
| phosphorylation | | ATP5C1 |

Enrichment Score: 0.9077271811850836

| | | |
|---|---|---|
| h_tnfr1Pathway: TNFR1 | 3 | CASP3, LMNA, PRKDC |
| Signaling Pathway | | |
| h_fasPathway: FAS | 3 | CASP3, LMNA, PRKDC |
| signaling pathway | | |
| ( CD95 ) | | |
| h_hivnefPathway: HIV-I | 3 | CASP3, LMNA, PRKDC |
| Nef: negative effector | | |
| of Fas and TNF | | |

Enrichment Score: 0.8974242711787185

| | | |
|---|---|---|
| Helicase | 6 | DDX39A, DDX3X, DHX15, |
| | | MCM4, MCM5, CHD3 |
| GO: 0004003~ATP- | 3 | DDX3X, MCM4, CHD3 |
| dependent DNA helicase | | |
| activity | | |
| GO: 0004386~helicase | 3 | DDX3X, MCM4, CHD3 |
| activity | | |

Enrichment Score: 0.8844871978021877

| | | |
|---|---|---|
| GO: 0005794~Golgi | 23 | TGOLN2, STX6, KDELR2, |
| apparatus | | USP8, ATP11B, NDFIP2, |
| | | RAB1B, TOPBP1, ARF5, |
| | | LMAN2, ERGIC3, PWP1, TAF11, |
| | | EI24, SP3, ARF3, MAPKAP1, |
| | | STX16, TMED10, USP33, FGD3, |
| | | DNM2, KAT6A |
| Golgi apparatus | 15 | TGOLN2, STX6, SNX9, ATP11B, |
| | | NDFIP2, ARF5, LMAN2, UXS1, |
| | | ERGIC3, COPB2, ARF3, STX16, |
| | | TMED10, ENTPD4, USP33 |
| GO: 0000139~Golgi | 10 | STX6, COPB2, KDELR2, ARF3, |
| membrane | | STX16, NDFIP2, TMED10, |
| | | RAB1B, LMAN2, DNM2 |

Enrichment Score: 0.8697007217508498

| | | |
|---|---|---|
| IPR016135: Ubiquitin- | 4 | UBE2N, UFC1, UBE2V2, |
| conjugating enzyme/RWD- | | UBE2L3 |
| like | | |
| GO: 0061631~ubiquitin | 3 | UBE2N, UBE2V2, UBE2L3 |
| conjugating enzyme | | |
| activity | | |
| IPR000608: Ubiquitin- | 3 | UBE2N, UBE2V2, UBE2L3 |
| conjugating enzyme, E2 | | |
| GO: 0016567~protein | 7 | KLHL7, UBE2N, NUB1, UBA6, |
| ubiquitination | | UBE2V2, UBE2L3, TRAF4 |

Enrichment Score: 0.8007359177589985

| | | |
|---|---|---|
| SM00312: PX | 3 | SNX9, PXK, SNX10 |
| domain: PX | 3 | SNX9, PXK, SNX10 |
| GO: | 4 | SNX9, PXK, ITPR3, SNX10 |
| 0035091~phosphatidylinositol | | |
| binding | | |
| IPR001683: Phoxhomologous | 3 | SNX9, PXK, SNX10 |
| domain | | |

Enrichment Score: 0.7905595110088506

| | | |
|---|---|---|
| Thiol protease | 6 | CASP3, USP8, EIF3F, USP10, |
| | | USP33, ALG13 |
| GO: 0004197~cysteine- | 4 | CASP3, USP8, USP10, USP33 |
| type endopeptidase activity | | |
| GO: 0004843~thiol- | 4 | USP8, EIF3F, USP10, USP33 |
| dependent ubiquitin-specific | | |
| protease activity | | |
| GO: 0016579~protein | 4 | USP8, EIF3F, USP10, USP33 |
| deubiquitination | | |
| IPR018200: Peptidase C19, | 3 | USP8, USP10, USP33 |
| ubiquitin carboxyl-terminal | | |
| hydrolase 2, conserved site | | |
| IPR001394: Peptidase C19, | 3 | USP8, USP10, USP33 |
| ubiquitin carboxyl-terminal | | |
| hydrolase 2 | | |

Enrichment Score: 0.7329253582520423

| | | |
|---|---|---|
| active site: Glycyl thioester | 5 | UBE2N, UFC1, UBA6, UBE2L3, |
| intermediate | | TRIP12 |
| GO: 0042787~protein | 5 | KLHL7, CUL4A, UBA6, |
| ubiquitination involved in | | UBE2L3, TRIP12 |
| ubiquitin-dependent | | |
| protein catabolic process | | |
| GO: 0006464~cellular | 4 | UBE2N, UBA6, PRKDC, |
| protein modification process | | UBE2L3 |
| hsa04120: Ubiquitin 5 | | UBE2N, CUL4A, UBA6, |
| mediated proteolysis | | UBE2L3, TRIP12 |
| GO: 0004842~ubiquitin- | 6 | KLHL7, UBE2N, UBE2L3, TTC3, |
| protein transferase activity | | TRIP12, TRAF4 |

Enrichment Score: 0.7159098666001777

| | | |
|---|---|---|
| Helicase | 6 | DDX39A, DDX3X, DHX15, |
| | | MCM4, MCM5, CHD3 |
| SM00490: HELICc | 4 | DDX39A, DDX3X, DHX15, |
| | | CHD3 |
| SM00487: DEXDc | 4 | DDX39A, DDX3X, DHX15, |
| | | CHD3 |
| domain: Helicase C-terminal | 4 | DDX39A, DDX3X, DHX15, |
| | | CHD3 |
| domain: Helicase ATP- | 4 | DDX39A, DDX3X, DHX15, |
| binding | | CHD3 |
| IPR001650: Helicase, | 4 | DDX39A, DDX3X, DHX15, |
| C-terminal | | CHD3 |
| IPR014001: Helicase, | 4 | DDX39A, DDX3X, DHX15, |
| superfamily 1/2, ATP-binding | | CHD3 |
| domain | | |
| GO: 0004004~ATP- | 3 | DDX39A, DDX3X, DHX15 |
| dependent RNA helicase | | |
| activity | | |
| IPR011545: DNA/RNA | 3 | DDX39A, DDX3X, DHX15 |
| helicase, DEAD/DEAH | | |
| box type, N-terminal | | |

Enrichment Score: 0.69498312277083

| | | |
|---|---|---|
| Aminoacyl-tRNA synthetase | 3 | AARS, HARS, VARS |
| GO: 0006418~tRNA | 3 | AARS, HARS, VARS |
| aminoacylation for protein | | |
| translation | | |
| Ligase | 8 | AARS, HARS, UBA6, CTPS1, |
| | | ASNS, VARS, TTC3, TRIP12 |
| hsa00970: Aminoacyl-tRNA | 3 | AARS, HARS, VARS |
| biosynthesis | | |

Enrichment Score: 0.5293095748164424

| | | |
|---|---|---|
| lipid moiety-binding region: | 5 | HPCAL1, GNAI2, ARF3, |
| N-myristoyl glycine | | PPP3R1, ARF5 |
| Myristate | 5 | HPCAL1, GNAI2, ARF3, |
| | | PPP3R1, ARF5 |
| GO: 0003924~GTPase | 6 | MFN2, DDX3X, GNAI2, ARF3, |
| activity | | ARF5, DNM2 |
| Lipoprotein | 10 | RGS10, HPCAL1, S1PR1, |
| | | GNAI2, ARF3, PPP3R1, LMNA, |
| | | RAB1B, STOML2, ARF5 |

Enrichment Score: 0.5161555683114979

| | | |
|---|---|---|
| IPR024156: Small GTPase | 3 | ARF3, ARL16, ARF5 |
| superfamily, ARF type | | |
| IPR006689: Small GTPase | 3 | ARF3, ARL16, ARF5 |
| superfamily, ARF/SAR type | | |
| nucleotide phosphate-binding | 7 | MFN2, GNAI2, ARF3, RAB1B, |
| region: GTP | | ARL16, ARF5, DNM2 |
| GO: 0003924~GTPase | 6 | MFN2, DDX3X, GNAI2, ARF3, |
| activity | | ARF5, DNM2 |
| GTP-binding | 7 | MFN2, GNAI2, ARF3, RAB1B, |
| | | ARL16, ARF5, DNM2 |
| GO: 0005525~GTP binding | 8 | MFN2, GNAI2, FKBP4, ARF3, |
| | | RAB1B, ARL16, ARF5, DNM2 |
| IPR005225: Small GTP- | 3 | ARF3, RAB1B, ARF5 |
| binding protein domain | | |
| GO: 0007264~small GTPase | 4 | ARF3, RAB1B, ARL16, ARF5 |
| mediated signal transduction | | |

Enrichment Score: 0.49661457171690665

| | | |
|---|---|---|
| IPR011990: | 7 | GPS1, NUB1, FKBP4, PPID, |
| Tetratricopeptide-like helical | | PRPF39, TTC3, PRPF6 |
| SM00028: TPR | 4 | FKBP4, PPID, TTC3, PRPF6 |
| IPR013026: | 4 | FKBP4, PPID, TTC3, PRPF6 |
| Tetratricopeptide repeat- | | |
| containing domain | | |
| IPR019734: | 4 | FKBP4, PPID, TTC3, PRPF6 |
| Tetratricopeptide repeat | | |
| repeat: TPR 3 | 4 | FKBP4, PPID, PRKDC, TTC3 |
| TPR repeat | 4 | FKBP4, PPID, PRKDC, TTC3 |
| repeat: TPR 2 | 4 | FKBP4, PPID, PRKDC, TTC3 |
| repeat: TPR 1 | 4 | FKBP4, PPID, PRKDC, TTC3 |

Enrichment Score: 0.46491176000453827

| | | |
|---|---|---|
| IPR002909: Cell surface | 3 | NFAT5, NFKB2, EXOC2 |
| receptor IPT/TIG | | |
| IPR014756: Immunoglobulin | 3 | NFAT5, NFKB2, EXOC2 |
| E-set | | |
| IPR013783: Immunoglobulin- | 4 | CRLF3, NFAT5, NFKB2, EXOC2 |
| like fold | | |

Enrichment Score: 0.4285527179810236

| | | |
|---|---|---|
| GO: 0016491~oxidoreductase | 6 | HSD17B10, KDM1A, AKR1A1, |
| activity | | TSTA3, KIAA1191, APEX1 |
| GO: 0055114~oxidation- | 12 | GLRX3, HSD17B10, KDM1A, |
| reduction process | | PYCR1, OXA1L, AKR1A1, |
| | | TSTA3, KIAA1191, APEX1, |
| | | BCO2, HIGD2A, SOD2 |
| Oxidoreductase | 10 | GCDH, HSD17B10, KDM1A, |
| | | PYCR1, AKR1A1, TSTA3, |
| | | KIAA1191, HADHA, BCO2, |
| | | SOD2 |
| NADP | 4 | PYCR1, AKR1A1, TSTA3, |
| | | KIAA1191 |

Enrichment Score: 0.36591445625467517

| | | |
|---|---|---|
| SM00249: PHD | 3 | PHF11, CHD3, KAT6A |
| IPR011011: Zinc finger, | 4 | PHF11, FGD3, CHD3, KAT6A |
| FYVE/PHD-type | | |
| IPR001965: Zinc finger, | 3 | PHF11, CHD3, KAT6A |
| PHD-type | | |
| IPR013083: Zinc finger, | 7 | PHF11, USP33, TTC3, TRAF4, |
| RING/FYVE/PHD-type | | FGD3, CHD3, KAT6A |

Enrichment Score: 0.25507693482602034

| | | |
|---|---|---|
| domain: SH3 | 4 | ITK, SNX9, HCLS1, SKAP1 |
| SM00326: SH3 | 4 | ITK, SNX9, HCLS1, SKAP1 |
| SH3 domain | 4 | ITK, SNX9, HCLS1, SKAP1 |
| IPR001452: Src homology-3 | 4 | ITK, SNX9, HCLS1, SKAP1 |
| domain | | |

Enrichment Score: 0.2406171678396523

| | | |
|---|---|---|
| SM00233: PH | 5 | ITK, DOK2, SKAP1, FGD3, |
| | | DNM2 |
| domain: PH | 5 | ITK, ADRM1, DOK2, SKAP1, |
| | | DNM2 |
| IPR001849: Pleckstrin | 5 | ITK, DOK2, SKAP1, FGD3, |
| homology domain | | DNM2 |
| IPR011993: Pleckstrin | 6 | ITK, DOK2, EPB41, SKAP1, |
| homology-like domain | | FGD3, DNM2 |

Enrichment Score: 0.1905601056044362

| | | |
|---|---|---|
| hsa04924: Renin secretion | 3 | GNAI2, PPP3R1, ITPR3 |
| hsa04724: Glutamatergic | 3 | GNAI2, PPP3R1, ITPR3 |
| synapse | | |
| hsa04921: Oxytocin signaling | 3 | GNAI2, PPP3R1, ITPR3 |
| pathway | | |
| hsa04022: cGMP-PKG | 3 | GNAI2, PPP3R1, ITPR3 |
| signaling pathway | | |

Enrichment Score: 0.1700736372529799

| | | |
|---|---|---|
| Kinase | 13 | ITK, PRPF4B, DTYMK, WNK1, |
| | | SMG1, PRKDC, AK2, PXK, |
| | | DOK2, CSNK2A1, RFK, |
| | | MAPKAP1, STK38L |
| GO: 0004672~protein kinase | 8 | PRPF4B, CSNK2A1, WNK1, |
| activity | | SMG1, PRKDC, PXK, CCL5, |
| | | STK38L |
| GO: 0004674~protein serine/ | 8 | PRPF4B, CSNK2A1, WNK1, |
| threonine kinase activity | | SMG1, PRKDC, CCNC, CPNE3, |
| | | STK38L |
| GO: 0018105~peptidyl-serine | 3 | SMG1, PRKDC, STK38L |
| phosphorylation | | |
| Serine/threonine-protein | 6 | PRPF4B, CSNK2A1, WNK1, |
| kinase | | SMG1, PRKDC, STK38L |
| IPR011009: Protein kinase- | 8 | ITK, PRPF4B, CSNK2A1, |
| like domain | | WNK1, SMG1, PRKDC, PXK, |
| | | STK38L |
| GO: 0006468~protein | 7 | PRPF4B, CSNK2A1, WNK1, |
| phosphorylation | | CCNC, CPNE3, PXK, STK38L |
| active site: Proton acceptor | 9 | GCDH, HSD17B10, ITK, |
| | | PRPF4B, CSNK2A1, WNK1, |
| | | ADSL, APEX1, STK38L |
| binding site: ATP | 7 | ITK, PRPF4B, CSNK2A1, RFK, |
| | | WNK1, VARS, STK38L |
| domain: Protein kinase | 6 | ITK, PRPF4B, CSNK2A1, |
| | | WNK1, PXK, STK38L |
| SM00220: S_TKc | 4 | PRPF4B, CSNK2A1, WNK1, |
| | | STK38L |
| IPR008271: Serine/threonine- | 4 | PRPF4B, CSNK2A1, WNK1, |
| protein kinase, active site | | STK38L |
| IPR000719: Protein kinase, | 6 | ITK, PRPF4B, CSNK2A1, |
| catalytic domain | | WNK1, PXK, STK38L |
| IPR017441: Protein kinase, | 3 | ITK, CSNK2A1, STK38L |
| ATP binding site | | |

Enrichment Score: 0.11135414381747946

| | | |
|---|---|---|
| topological domain: | 10 | KDELR2, SEC11A, ALG5, |
| Lumenal | | TMED10, SPCS1, LMAN2, |
| | | ENTPD4, UXS1, SSR2, |
| | | ERGIC3 |
| topological domain: | 21 | TGOLN2, STX6, KDELR2, |
| Cytoplasmic | | SEC11A, GPR171, ATP11B, |
| | | ALG5, CD99, LMAN2, ITPR3, |
| | | UXS1, ERGIC3, MFN2, S1PR1, |
| | | STX16, TMEM170A, TMED10, |
| | | SPCS1, ENTPD4, SSR2, HIGD2A |
| signal peptide | 15 | TGOLN2, HYOU1, CST7, |
| | | ERP29, CNPY3, CCDC47, |
| | | TMED10, CD99, LRCH3, |
| | | MESDC2, HSPA13, LMAN2, |
| | | CCL5, SSR2, SOD2 |
| Glycoprotein | 23 | TGOLN2, EPB41, CWC27, |
| | | GPR171, WNK1, CNPY3, ALG5, |
| | | CCDC47, CD99, MESDC2, |
| | | LMAN2, CCL5, UXS1, RBMX, |
| | | ERGIC3, HYOU1, S1PR1, |
| | | NUP62, CST7, TMEM170A, |
| | | TMED10, ENTPD4, SSR2 |
| Signal | 18 | TGOLN2, ERP29, WNK1, |
| | | CNPY3, CCDC47, CD99, |
| | | MESDC2, LMAN2, CCL5, |
| | | HYOU1, RAE1, CST7, TMED10, |
| | | ABHD10, LRCH3, HSPA13, |
| | | BCO2, SSR2 |
| glycosylation site: N-linked | 17 | TGOLN2, CWC27, GPR171, |
| (GlcNAc . . . ) | | CNPY3, ALG5, CCDC47, |
| | | MESDC2, LMAN2, UXS1, |
| | | ERGIC3, HYOU1, S1PR1, CST7, |
| | | TMEM170A, TMED10, ENTPD4, |
| | | SSR2 |

Enrichment Score: 0.0903744092851981

| | | |
|---|---|---|
| repeat: 5 | 3 | TGOLN2, NUP62, DNMT1 |
| repeat: 4 | 3 | TGOLN2, NUP62, DNMT1 |
| repeat: 3 | 3 | TGOLN2, NUP62, DNMT1 |
| repeat: 1 | 3 | TGOLN2, NUP62, DNMT1 |
| repeat: 2 | 3 | TGOLN2, NUP62, DNMT1 |

Enrichment Score: 0.069370372136031

| | | |
|---|---|---|
| GO: 0098609~cell-cell | 4 | SNX9, USP8, DDX3X, IST1 |
| adhesion | | |
| GO: 0098641~cadherin | 4 | SNX9, USP8, DDX3X, IST1 |
| binding involved in cell-cell | | |
| adhesion | | |
| GO: 0005913~cell-cell | 4 | SNX9, USP8, DDX3X, IST1 |
| adherens junction | | |

Enrichment Score: 0.05747010288539574

| | | |
|---|---|---|
| domain: EF-hand 2 | 3 | HPCAL1, EFCAB14, PPP3R1 |
| domain: EF-hand 1 | 3 | HPCAL1, EFCAB14, PPP3R1 |
| IPR002048: EF-hand | 3 | HPCAL1, EFCAB14, PPP3R1 |
| domain | | |
| IPR011992: EF-hand-like | 3 | HPCAL1, EFCAB14, PPP3R1 |
| domain | | |
| Calcium | 7 | HPCAL1, EFCAB14, PPP3R1, |
| | | TKT, LMAN2, ENTPD4, ITPR3 |
| GO: 0005509~calcium ion | 5 | HPCAL1, EFCAB14, PPP3R1, |
| binding | | CCDC47, ITPR3 |

Enrichment Score: 0.04306870373594361

| | | |
|---|---|---|
| IPR013083: Zinc finger, | 7 | PHF11, USP33, TTC3, TRAF4, |
| RING/FYVE/PHD-type | | FGD3, CHD3, KAT6A |
| GO: 0008270~zinc ion | 13 | POLR2K, TIM M9, AARS, |
| binding | | TIMM10, DNMT1, PHF11, |
| | | TCEA1, SREK1IP1, USP33, |
| | | TTC3, TRAF4, CHD3, KAT6A |
| Zinc | 24 | VP529, ITK, POLR2K, AARS, |
| | | TIMM10, NOB1, PHF11, TTC3, |
| | | SUZ12, PRIM1, LAP3, PSMD14, |
| | | RFK, SP3, TIM M9, DNMT1, |
| | | TCEA1, SREK1IP1, USP33, |
| | | FGD3, TRAF4, GPATCH8, |
| | | CHD3, KAT6A |
| Zinc-finger | 15 | ITK, POLR2K, PHF11, TTC3, |
| | | SUZ12, SP3, DNMT1, TCEA1, |
| | | SREK1IP1, USP33, TRAF4, |
| | | GPATCH8, FGD3, CHD3, |
| | | KAT6A |

Enrichment Score: 9.170272943712669E−6

| | | |
|---|---|---|
| Membrane | 78 | TGOLN2, OXA1L, COA3, |
| | | UTP18, CAPZA2, DNAJB14, |
| | | ALG5, STOML2, RAB1B, |
| | | LINC00116, SKAP1, UXS1, |
| | | THADA, NDUFS6, COPB2, |
| | | ATAD3B, S1PR1, ELOVL5, |
| | | DNAJC9, MAPKAP1, TIMM9, |
| | | YRDC, STX6, KDELR2, BCL10, |
| | | GPR171, CCDC47, TMEM126B, |
| | | ERGIC3, MFN2, EIF5AL1, |
| | | TMEM170A, ATP5C1, AAMP, |
| | | LRCH3, SLC25A39, VPS26B, |
| | | SNX10, HIGD2A, VPS29, SNX9, |
| | | USP8, NDUFB6, GNAI2, CRLF3, |
| | | PPP3R1, TIMM10, UBA6, ARF5, |
| | | LMAN2, PXK, PTRH2, DDX3X, |
| | | STX16, NFAT5, TMED10, |
| | | ENTPD4, STK38L, TRAF4, |
| | | NDUFA2, HPCAL1, SMCHD1, |
| | | FIBP, HCLS1, SEC11A, |
| | | TMEM120B, FDPS, ATP11B, |
| | | CD99, TOMM40, NDFIP2, |
| | | ITPR3, EI24, SPCS1, CPNE3, |
| | | TEX10, SSR2, DNM2 |
| GO: 0016021~integral | 46 | TGOLN2, OXA1L, NDUFB6, |
| component of membrane | | COA3, CRLF3, UTP18, CAPZA2, |
| | | DNAJB14, UBA6, ALG5, |
| | | LINC00116, PTRH2, UXS1, |
| | | THADA, S1PR1, ELOVL5, |
| | | STX16, NFAT5, TMED10, |
| | | ENTPD4, STX6, KDELR2, |
| | | SMCHD1, SEC11A, GPR171, |
| | | TMEM120B, ATP11B, FDPS, |
| | | NDFIP2, CCDC47, TOMM40, |
| | | CD99, TMEM126B, ITPR3, |
| | | ERGIC3, MFN2, EI24, |
| | | TMEM170A, TCEB3, LRCH3, |
| | | SPCS1, SLC25A39, VPS26B, |
| | | TEX10, SSR2, HIGD2A |
| Transmembrane | 46 | TGOLN2, OXA1L, NDUFB6, |
| | | COA3, CRLF3, UTP18, CAPZA2, |
| | | DNAJB14, UBA6, ALG5, |
| | | LINC00116, LMAN2, PTRH2, |
| | | UXS1, THADA, S1PR1, |
| | | ELOVL5, STX16, NFAT5, |
| | | TMED10, ENTPD4, STX6, |
| | | KDELR2, SMCHD1, SEC11A, |
| | | GPR171, TMEM120B, ATP11B, |
| | | FDPS, NDFIP2, CCDC47, |
| | | TOMM40, CD99, TMEM126B, |
| | | ITPR3, ERGIC3, MFN2, EI24, |
| | | TMEM170A, LRCH3, SPCS1, |
| | | SLC25A39, VPS26B, TEX10, |
| | | SSR2, HIGD2A |
| Transmembrane helix | 45 | TGOLN2, OXA1L, NDUFB6, |
| | | COA3, UTP18, CRLF3, CAPZA2, |
| | | DNAJB14, UBA6, ALG5, |
| | | LINC00116, LMAN2, PTRH2, |
| | | UXS1, THADA, S1PR1, |
| | | ELOVL5, STX16, NFAT5, |
| | | TMED10, ENTPD4, STX6, |
| | | KDELR2, SMCHD1, SEC11A, |
| | | GPR171, TMEM120B, ATP11B, |
| | | FDPS, NDFIP2, CCDC47, CD99, |
| | | TMEM126B, ITPR3, ERGIC3, |
| | | MFN2, EI24, TMEM170A, |
| | | LRCH3, SPCS1, SLC25A39, |
| | | VPS26B, TEX10, SSR2, HIGD2A |
| topological domain: | 21 | TGOLN2, STX6, KDELR2, |
| Cytoplasmic | | SEC11A, GPR171, ATP11B, |
| | | ALG5, CD99, LMAN2, ITPR3, |
| | | UXS1, ERGIC3, MFN2, S1PR1, |
| | | STX16, TMEM170A, TMED10, |
| | | SPCS1, ENTPD4, SSR2, HIGD2A |
| transmembrane region | 35 | TGOLN2, OXA1L, NDUFB6, |
| | | COA3, DNAJB14, ALG5, |
| | | LINC00116, LMAN2, UXS1, |
| | | S1PR1, ELOVL5, STX16, |
| | | TMED10, ENTPD4, SREK1IP1, |
| | | STX6, KDELR2, SEC11A, |
| | | GPR171, TMEM120B, ATP11B, |
| | | NDFIP2, CCDC47, CD99, |
| | | TMEM126B, ITPR3, ERGIC3, |
| | | MFN2, EI24, TMEM170A, |
| | | SPCS1, SLC25A39, SSR2, |
| | | TEX10, HIGD2A |
| Acetylation | 143 | MRPS33, PRPF4B, CAPZA2, |
| | | DTYMK, RAB1B, STOML2, |
| | | FOXO1, PRIM1, TOP1, GTF2E2, |
| | | S1PR1, ELOVL5, VPS13D, |
| | | TIMM9, EIF1, PTBP3, PPP4C, |
| | | SUPT5H, DDX39A, BCL10, |
| | | AARS, ERGIC3, ADRM1, |
| | | PYCR1, DCUN1D1, EIF2S1, |
| | | CLPP, ATP5C1, HARS, ADSL, |
| | | HIGD2A, SNX9, MEAF6, |
| | | HSD17B10, UBA6, CTPS1, |
| | | UBE2V2, BANF1, HADHA, |
| | | PSMB4, POLE4, DDX3X, |
| | | HTATSF1, MTPN, EIF3F, |
| | | STK38L, TFDP1, PRPF40A, |
| | | GCDH, ARL14EP, SMCHD1, |
| | | FIBP, CCDC25, MMADHC, |
| | | FDPS, AK2, SMG1, TKT, DENR, |
| | | TPD52L2, UBE2L3, RBMX, |
| | | PTPN11, HYOU1, EI24, NUP62, |
| | | PSMC4, PPID, METTL10, |
| | | WDR4, BABAM1, RBMXL1, |
| | | DNMT1, EIF4E2, GPATCH8, |
| | | KAT6A, SRSF1, HNRNPA1L2, |
| | | COA3, TCOF1, BOD1L1, |
| | | COPB2, NDUFS6, CASP3, |
| | | ATAD3B, MAPKAP1, USP10, |
| | | DDA1, STX6, NCOA7, POLR1C, |
| | | RBBP7, MCM4, MCM5, PURA, |
| | | UBE2N, TAF11, LAP3, KPNA3, |
| | | THOC1, VPS29, GLRX3, NXT1, |
| | | NDUFB6, CRLF3, FKBP4, |
| | | FKBP3, RNH1, RPL35, PRKDC, |
| | | ASNS, VARS, HPRT1, SRRT, |
| | | MRPL13, FAM107B, AKR1A1, |
| | | CD2BP2, REXO2, DHX15, |
| | | NFAT5, GTF3C6, TCEA1, |
| | | APEX1, EXOC2, TRIP12, |
| | | NDUFA2, CWC27, HCLS1, |
| | | LMNA, ACLY, ETF1, COTL1, |
| | | SMC2, SMC3, SOD2, PPP1R2, |
| | | SP3, CKS2, TCEB3, PSAT1, |
| | | SSNA1, DNM2 |
| Proteomics identification | 235 | TGOLN2, OXA1L, DNAJB14, |
| | | RAB1B, STOML2, UXS1, |
| | | PRIM1, C1ORF109, WDR73, |
| | | VPS13D, RAE1, EIF1AY, PTBP3, |
| | | SREK1IP1, SUPT5H, RCOR1, |
| | | AARS, WNK1, CCDC137, |
| | | ERGIC3, EIF2S1, HARS, ADSL, |
| | | LRCH3, MRPL48, CPSF3L, |
| | | HSD17B10, GNAI2, PABPC4, |
| | | UBA6, PXK, ARF5, BANF1, |
| | | HADHA, HTATSF1, STX16, |
| | | CDC40, TMED10, PRPF40A, |
| | | GCDH, HPCAL1, TMEM120B, |
| | | ATP11B, NDFIP2, YTHDC1, |
| | | TKT, TPD52L2, GCFC2, |
| | | PTPN11, HYOU1, EI24, NUP62, |
| | | MLX, METTL10, UTP14A, |
| | | SNRNP25, ALG13, BCO2, |
| | | KAT6A, SRSF1, UTP18, TCOF1, |
| | | NFKB2, RPS19BP1, BOD1L1, |
| | | COPB2, WDR36, USP10, |
| | | KDELR2, MRPL4, CNPY3, |
| | | PRPF39, POLR1C, CCNC, |
| | | MESDC2, RBBP7, DOK2, |
| | | THOC1, USP8, POLR2K, NOB1, |
| | | RPL35, SRRT, MRPL13, CIR1, |
| | | REXO2, TCEA1, TSTA3, USP33, |
| | | EXOC2, TRIP12, NUB1, CWC27, |
| | | HCLS1, LMNA, ACLY, PHF11, |
| | | ETF1, SOD2, PSMD14, MRPL22, |
| | | PPP1R2, MRPL28, IST1, TCEB3, |
| | | SSNA1, SSR2, TEX10, DNM2, |
| | | MRPS35, MRPS33, PRPF4B, |
| | | CAPZA2, DTYMK, RBM6, |
| | | TCEAL8, SRP19, CNOT7, MAF1, |
| | | KLHL7, KDM1A, GTF2E2, |
| | | CSNK2A1, NUDCD1, ELOVL5, |
| | | TIMM9, EIF1, PPP4C, IFRD2, |
| | | DDX39A, BCL10, ERP29, |
| | | HMCES, ARL16, TOPBP1, |
| | | TMEM126B, SUZ12, ADRM1, |
| | | PYCR1, DCUN1D1, SMARCE1, |
| | | RFK, CLPP, SLC25A39, VPS26B, |
| | | SNRPG, SNX9, MEAF6, |
| | | UBE2V2, LMAN2, PTRH2, |
| | | CCL5, FAM207A, TTC3, PSMB7, |
| | | DDX3X, MTPN, EIF3F, |
| | | ENTPD4, STK38L, TRAF4, |
| | | TFDP1, ITK, ARL14EP, |
| | | SMCHD1, FIBP, CCDC25, |
| | | EPB41, MMADHC, SEC11A, |
| | | FDPS, AK2, TOMM40, SMG1, |
| | | THUMPD2, DENR, RBMX, |
| | | PPID, BABAM1, DNMT1, |
| | | CPNE3, SPCS1, EIF4E2, |
| | | GPATCH8, COA3, SKAP1, |
| | | THADA, NDUFS6, CASP3, |
| | | MAPKAP1, ABHD10, FTSJ3, |
| | | METTL5, CCDC47, NCOA7, |
| | | MCM4, MCM5, LAP3, MFN2, |
| | | UBE2N, NCOA4, PPM1K, |
| | | AAMP, KPNA3, KIAA1191, |
| | | SNX10, C1D, VPS29, NDUF136, |
| | | CRLF3, FKBP4, FKBP3, PPP3R1, |
| | | RNH1, PRKDC, ASNS, VARS, |
| | | AKR1A1, FAM107B, UFM1, |
| | | CCDC124, NFAT5, APEX1, |
| | | CHD3, GPS1, CD99, COTL1, |
| | | SMC2, RSBN1, SLBP, PWP1, |
| | | PSMG4, CUL4A, SP3, CENPV |
| Phosphoprotein | 193 | TGOLN2, OXA1L, STOML2, |
| | | RAB1B, UXS1, S1PR1, RAE1, |
| | | VPS13D, PTBP3, SUPT5H, |
| | | SREK1IP1, EFCAB14, RCOR1, |
| | | AARS, WNK1, KRT10, |
| | | CCDC137, ERGIC3, EIF2S1, |
| | | HARS, ATP5C1, ADSL, LRCH3, |
| | | MCTS1, NOC3L, PABPC4, |
| | | UBA6, BANF1, HADHA, VPS72, |
| | | HTATSF1, CDC40, STX16, |
| | | PRPF40A, ATP11B, NDFIP2, |
| | | YTHDC1, TKT, TPD52L2, |
| | | GCFC2, PTPN11, HYOU1, EI24, |
| | | NUP62, MLX, METTL10, |
| | | RBMXL1, UTP14A, KAT6A, |
| | | SRSF1, UTP18, TCOF1, NFKB2, |
| | | RPS19BP1, BOD1L1, COPB2, |
| | | WDR36, USP10, STX6, PRPF39, |
| | | CCNC, POLR1C, RBBP7, |
| | | NOC2L, DOK2, THOC1, USP8, |
| | | RPL35, NOB1, HPRT1, NUFIP2, |
| | | SRRT, CIR1, CD2BP2, REXO2, |
| | | DHX15, TCEA1, USP33, TRIP12, |
| | | EXOC2, HCLS1, CWC27, |
| | | LMNA, ACLY, ITPR3, ETF1, |
| | | PSMD14, PPP1R2, IST1, TCEB3, |
| | | PSAT1, TEX10, DNM2, PRPF4B, |
| | | CAPZA2, FOXO1, RBM6, MAF1, |
| | | TOP1, KDM1A, GTF2E2, |
| | | CSNK2A1, ELOVL5, NUDCD1, |
| | | DNAJC9, EIF1, DDX39A, |
| | | BCL10, ERP29, HMCES, |
| | | TMEM126B, TOPBP1, SUZ12, |
| | | PYCR1, ADRM1, SMARCE1, |
| | | VPS26B, MEAF6, SNX9, CTPS1, |
| | | TTC3, FAM207A, PSMB4, |
| | | POLE4, DDX3X, MTPN, EIF3F, |
| | | ASF1A, STK38L, TRAF4, FGD3, |
| | | FDP1, ARL14EP, ITK, SMCHD1, |
| | | CCDC25, EPB41, SMG1, AK2, |
| | | DENR, RBMX, PSMC4, PPID, |
| | | BABAM1, WDR4, DNMT1, |
| | | CPNE3, GPATCH8, EIF4E2, |
| | | HNRNPA1L2, SKAP1, THADA, |
| | | CASP3, MAPKAP1, YRDC, |
| | | DDA1, FTSJ3, NCOA7, MCM4, |
| | | MCM5, PRPF6, PURA, LAP3, |
| | | MIN2, PPM1K, AAMP, |
| | | KIAA1191, KPNA3, C1D, |
| | | GLRX3, FKBP4, FKBP3, |
| | | PPP3R1, RNH1, PRKDC, ASNS, |
| | | VARS, RGS10, AKR1A1, |
| | | CCDC124, GTF3C6, NFAT5, |
| | | APEX1, CHD3, GPS1, CD99, |
| | | RSBN1, SMC3, PWP1, SLBP, |
| | | CUL4A, SP3, CENPV |
| GO: 0044822~poly(A) RNA | 63 | SRSF1, MRPS35, PRPF4B, |
| binding | | UTP18, TCOF1, RBM6, SRP19, |
| | | RPS19BP1, TOP1, GTF2E2, |
| | | WDR36, USP10, PTBP3, EIF1, |
| | | SUPT5H, FTSJ3, DDX39A, |
| | | MRPL4, CCDC47, CCDC137, |
| | | PURA, NOC2L, PRPF6, UBE2N, |
| | | EIF2S1, ATP5C1, SNRPG, |
| | | GLRX3, HSD17B10, FKBP4, |
| | | NOC3L, FKBP3, PABPC4, |
| | | RPL35, PRKDC, NUFIP2, SRRT, |
| | | MRPL13, DDX3X, CCDC124, |
| | | HTATSF1, CDC40, MRPL54, |
| | | DHX15, APEX1, CHD3, |
| | | PRPF40A, MRPS26, FDPS, |
| | | SMG1, YTHDC1, TPD52L2, |
| | | ETF1, UBE2L3, RBMX, SLBP, |
| | | MRPL22, MRPL28, CPNE3, |
| | | UTP14A, ALG13, EIF4E2, |
| | | GPATCH8 |
| GO: 0005654~nucleoplasm | 100 | TGOLN2, PRPF4B, FOXO1, |
| | | MAF1, CNOT7, KLHL7, PRIM1, |
| | | TOP1, KDM1A, GTF2E2, |
| | | CSNK2A1, SUPT5H, PPP4C, |
| | | DDX39A, RCOR1, TOPBP1, |
| | | SUZ12, ADRM1, SMARCE1, |
| | | SNRPG, CPSF3L, MEAF6, |
| | | GNAI2, UBE2V2, BANF1, |
| | | VPS72, PSMB4, PSMB7, |
| | | HTATSF1, CDC40, ASF1A, |
| | | TFDP1, PRPF40A, MRPS26, |
| | | FDPS, TOMM40, TKT, RBMX, |
| | | GCFC2, PSMC4, PPID, |
| | | BABAM1, WDR4, DNMT1, |
| | | UTP14A, SNRNP25, KAT6A, |
| | | SRSF1, UTP18, NFKB2, |
| | | RPS19BP1, BOD1L1, CASP3, |
| | | WDR36, MAPKAP1, USP10, |
| | | CCNC, POLR1C, RBBP7, |
| | | MCM4, MCM5, PRPF6, NOC2L, |
| | | UBE2N, TAF11, LAP3, KPNA3, |
| | | C1D, THOC1, NXT1, NDUFB6, |
| | | USP8, POLR2K, FKBP4, RNH1, |
| | | NOB1, PPP3R1, PRKDC, SRRT, |
| | | CD2BP2, GTF3C6, NFAT5, |
| | | TCEA1, APEX1, USP33, TRIP12, |
| | | CHD3, GPS1, LMNA, ACLY, |
| | | ITPR3, SMC2, SMC3, SLBP, |
| | | PSMD14, CUL4A, SP3, CENPV, |
| | | TCEB3, TEX10 |
| Nucleus | 128 | PRPF4B, RBM6, FOXO1, |
| | | TCEAL8, CNOT7, MAF1, |
| | | KLHL7, KDM1A, TOP1, |
| | | GTF2E2, CSNK2A1, NUDCD1, |
| | | RAE1, DNAJC9, PPP4C, |
| | | SUPT5H, DDX39A, RCOR1, |
| | | TOPBP1, SUZ12, ADRM1, |
| | | DCUN1D1, SMARCE1, |
| | | TMEM170A, SNRPG, CPSF3L, |
| | | MEAF6, NOC3L, BANF1, TTC3, |
| | | VPS72, PSMB4, POLE4, PSMB7, |
| | | DDX3X, MTPN, HTATSF1, |
| | | CDC40, ASF1A, TRAF4, TFDP1, |
| | | PRPF40A, FIBP, EPB41, |
| | | TMEM120B, YTHDC1, SMG1, |
| | | UBE2L3, RBMX, GCFC2, |
| | | PTPN11, EI24, NUP62, PSMC4, |
| | | PPID, MLX, WDR4, BABAM1, |
| | | RBMXL1, DNMT1, CPNE3, |
| | | UTP14A, SNRNP25, KAT6A, |
| | | HNRNPA1L2, SRSF1, UTP18, |
| | | TCOF1, NFKB2, SKAP1, |
| | | RPS19BP1, WDR36, MAPKAP1, |
| | | USP10, FTSJ3, NCOA7, PRPF39, |
| | | CCNC, POLR1C, RBBP7, |
| | | MCM4, MCM5, PURA, PRPF6, |
| | | NOC2L, UBE2N, TAF11, |
| | | EIF5AL1, KPNA3, THOC1, C1D, |
| | | NXT1, USP8, POLR2K, FKBP4, |
| | | FKBP3, NOB1, PRKDC, NUFIP2, |
| | | RGS10, SRRT, CIR1, CD2BP2, |
| | | UFM1, REXO2, DHX15, NFAT5, |
| | | GTF3C6, TCEA1, APEX1, |
| | | TRIP12, CHD3, GPS1, NUB1, |
| | | LMNA, PHF11, COTL1, SMC2, |
| | | SMC3, RSBN1, SLBP, PWP1, |
| | | IST1, SP3, CENPV, TCEB3, |
| | | SSNA1, TEX10 |
| Ubl conjugation | 59 | SRSF1, PRPF4B, TCOF1, RBM6, |
| | | FOXO1, NFKB2, MAF1, |
| | | BOD1L1, TOP1, EIF1AY, USP10, |
| | | SUPT5H, DDX39A, AARS, |
| | | WNK1, HMCES, TOPBP1, |
| | | RBBP7, MCM4, UBE2N, SUZ12, |
| | | MFN2, ADRM1, SMARCE1, |
| | | ADSL, C1D, THOC1, SNX9, |
| | | MEAF6, USP8, FKBP4, PRKDC, |
| | | PTRH2, HPRT1, NUFIP2, |
| | | DDX3X, UFM1, NFAT5, TCEA1, |
| | | USP33, APEX1, TRAF4, CHD3, |
| | | PRPF40A, ITK, SMCHD1, |
| | | LMNA, NDFIP2, YTHDC1, |
| | | ACLY, UBE2L3, RBMX, RSBN1, |
| | | CUL4A, SP3, DNMT1, RBMXL1, |
| | | UTP14A, EIF4E2 |
| GO: 0005737~cytoplasm | 127 | MRPS35, FOXO1, SRP19, MAF1, |
| | | KLHL7, GTF2E2, NUDCD1, |
| | | RAE1, DNAJC9, EIF1, PDRG1, |
| | | PPP4C, DDX39A, BCL10, AARS, |
| | | WNK1, KRT10, TOPBP1, |
| | | ADRM1, RFK, CST7, EIF2S1, |
| | | HARS, LRCH3, MCTS1, |
| | | CPSF3L, SNX9, MEAF6, |
| | | HSD17B10, GNAI2, PABPC4, |
| | | UBA6, UBE2V2, ARF5, PXK, |
| | | CCL5, BANF1, TTC3, PSMB4, |
| | | PSMB7, DDX3X, STX16, |
| | | STK38L, TRAF4, FGD3, |
| | | PRPF40A, ARL14EP, EPB41, |
| | | MMADHC, FDPS, NDFIP2, |
| | | TOMM40, SMG1, TPD52L2, |
| | | UBE2L3, PTPN11, EI24, NUP62, |
| | | PSMC4, PPID, MLX, METTL10, |
| | | WDR4, BABAM1, DNMT1, |
| | | CPNE3, EIF4E2, SNRNP25, |
| | | SRSF1, HNRNPA1L2, TCOF1, |
| | | UFC1, NFKB2, SKAP1, |
| | | RPS19BP1, CASP3, MAPKAP1, |
| | | USP10, YRDC, PURA, NOC2L, |
| | | UBE2N, LAP3, AAMP, KPNA3, |
| | | KIAA1191, THOC1, C1D, |
| | | VPS29, NXT1, USP8, CRLF3, |
| | | FKBP4, RNH1, RPL35, HPRT1, |
| | | NUFIP2, SRRT, CIR1, CCDC124, |
| | | CD2BP2, UFM1, DHX15, |
| | | NFAT5, TSTA3, USP33, APEX1, |
| | | TRIP12, CHD3, GPS1, NUB1, |
| | | HCLS1, LMNA, CD99, ACLY, |
| | | COTL1, ITPR3, ETF1, SMC2, |
| | | SMC3, SLBP, MRPL28, SP3, |
| | | CENPV, PSAT1, TEX10, DNM2 |
| Protein biosynthesis | 16 | AARS, DENR, VARS, ETF1, |
| | | MTIF3, EIF2S1, EIF5AL1, EIF3F, |
| | | EIF1AY, HARS, TCEB3, TCEA1, |
| | | EIF1, SUPT5H, MCTS1, EIF4E2 |
| mRNA splicing | 20 | HNRNPA1L2, SRSF1, DDX39A, |
| | | PRPF4B, YTHDC1, PRPF39, |
| | | RBMX, GCFC2, PRPF6, CIR1, |
| | | CD2BP2, CDC40, DHX15, |
| | | RBMXL1, PTBP3, SREK1IP1, |
| | | SNRNP25, THOC1, SNRPG, |
| | | PRPF40A |
| Mitochondrion | 43 | HSD17B10, MRPS35, OXA1L, |
| | | NDUFB6, MRPS33, COA3, |
| | | FKBP4, TIMM10, STOML2, |
| | | PTRH2, MTIF3, HADHA, |
| | | NDUFS6, MRPL13, ATAD3B, |
| | | DDX3X, TIMM9, REXO2, |
| | | MRPL54, ABHD10, YRDC, |
| | | APEX1, GCDH, MRPS26, |
| | | MRPL4, NDUFA2, MMADHC, |
| | | HCLS1, AK2, TOMM40, |
| | | TMEM126B, SOD2, MFN2, |
| | | PYCR1, MRPL22, MRPL28, |
| | | PPM1K, CLPP, ATP5C1, |
| | | MRPL48, SLC25A39, BCO2, |
| | | HIGD2A |
| Isopeptide bond | 43 | SRSF1, MEAF6, PRPF4B, |
| | | FKBP4, TCOF1, RBM6, NFKB2, |
| | | PTRH2, MAF1, HPRT1, NUFIP2, |
| | | BOD1L1, TOP1, DDX3X, UFM1, |
| | | EIF1AY, NFAT5, TCEA1, |
| | | SUPT5H, PRPF40A, CHD3, |
| | | DDX39A, SMCHD1, HMCES, |
| | | LMNA, YTHDC1, ACLY, |
| | | RBBP7, RBMX, RSBN1, UBE2N, |
| | | SUZ12, ADRM1, CUL4A, |
| | | SMARCE1, SP3, RBMXL1, |
| | | ADSL, DNMT1, UTP14A, |
| | | EIF4E2, THOC1, C1D |
| GO: 0005515~protein binding | 145 | OXA1L, PRPF4B, RAB1B, |
| | | STOML2, FOXO1, CNOT7, |
| | | KDM1A, C1ORF109, TOP1, |
| | | GTF2E2, CSNK2A1, ELOVL5, |
| | | PPP4C, SUPT5H, SREK1IP1, |
| | | DDX39A, BCL10, RCOR1, |
| | | ARL16, TOPBP1, SUZ12, |
| | | ADRM1, PYCR1, DCUN1D1, |
| | | SMARCE1, EIF2S1, CLPP, |
| | | TMEM170A, MRPL48, SNRPG, |
| | | CPSF3L, SNX9, MEAF6, |
| | | HSD17B10, GNAI2, UBA6, |
| | | UBE2V2, ARF5, PTRH2, CCL5, |
| | | BANF1, TTC3, VPS72, POLE4, |
| | | PSMB7, DDX3X, STX16, EIF3F, |
| | | TMED10, STK38L, ASF1A, |
| | | TRAF4, TFDP1, PRPF40A, ITK, |
| | | HPCAL1, EPB41, TSR2, |
| | | MMADHC, ATP11B, YTHDC1, |
| | | NDFIP2, SMG1, UBE2L3, |
| | | RBMX, PTPN11, PSMC4, |
| | | NUP62, PPID, WDR4, BABAM1, |
| | | DNMT1, SPCS1, CPNE3, |
| | | HSPA13, EIF4E2, SNRNP25, |
| | | KAT6A, SRSF1, COA3, UFC1, |
| | | NFKB2, SKAP1, RPS19BP1, |
| | | CASP3, USP10, STX6, CCNC, |
| | | POLR1C, RBBP7, MCM4, |
| | | MCM5, PURA, PRPF6, NOC2L, |
| | | MFN2, UBE2N, TAF11, PPM1K, |
| | | KPNA3, SNX10, THOC1, C1D, |
| | | VPS29, GLRX3, NXT1, USP8, |
| | | CRLF3, FKBP4, RNH1, PPP3R1, |
| | | PRKDC, HPRT1, VARS, |
| | | NUFIP2, SRRT, MRPL13, CIR1, |
| | | CD2BP2, DHX15, GTF3C6, |
| | | USP33, APEX1, EXOC2, TRIP12, |
| | | CHD3, NUB1, HCLS1, LMNA, |
| | | ACLY, ITPR3, ETF1, SMC2, |
| | | SMC3, SLBP, PSMD14, PPP1R2, |
| | | CUL4A, MRPL28, IST1, SP3, |
| | | CKS2, TCEB3, SSNA1, DNM2 |
| mRNA processing | 21 | HNRNPA1L2, SRSF1, DDX39A, |
| | | PRPF4B, YTHDC1, PRPF39, |
| | | RBMX, GCFC2, SLBP, PRPF6, |
| | | CIR1, CD2BP2, CDC40, DHX15, |
| | | RBMXL1, PTBP3, SREK1IP1, |
| | | SNRNP25, THOC1, SNRPG, |
| | | PRPF40A |
| GO: 0005739~mitochondrion | 47 | HSD17B10, MRPS35, OXA1L, |
| | | NDUFB6, MRPS33, COA3, |
| | | FKBP4, DTYMK, PPP3R1, |
| | | TIMM10, FOXO1, RAB1B, |
| | | PTRH2, SRP19, VARS, UXS1, |
| | | MTIF3, HADHA, TIMM9, |
| | | REXO2, ABHD10, YRDC, |
| | | APEX1, TFDP1, GCDH, |
| | | MRPS26, MRPL4, FIBP, |
| | | MMADHC, HCLS1, AARS, |
| | | NDFIP2, TOMM40, PTPN11, |
| | | SOD2, MIN2, LAP3, PYCR1, |
| | | MRPL22, MRPL28, RFK, |
| | | PPM1K, CLPP, HARS, ATP5C1, |
| | | ADSL, BCO2 |
| GO: 0005743~mitochondrial | 24 | MRPS35, MRPS26, MRPL4, |
| inner membrane | | NDUFA2, OXA1L, MRPS33, |
| | | NDUFB6, TIMM10, STOML2, |
| | | AK2, TMEM126B, HADHA, |
| | | SOD2, NDUFS6, MRPL22, |
| | | MRPL13, ATAD3B, MRPL28, |
| | | TIMM9, MRPL54, ATP5C1, |
| | | MRPL48, SLC25A39, HIGD2A |
| Cytoplasm | 106 | RAB1B, STOML2, FOXO1, |
| | | CNOT7, MAF1, SRP19, WDR73, |
| | | NUDCD1, RAE1, DNAJC9, |
| | | PDRG1, PPP4C, DDX39A, |
| | | BCL10, AARS, WNK1, TOPBP1, |
| | | ADRM1, RFK, CST7, HARS, |
| | | VPS26B, MCTS1, SNRPG, |
| | | CPSF3L, SNX9, GNAI2, |
| | | PABPC4, ARF5, PXK, BANF1, |
| | | PSMB4, PSMB7, DDX3X, |
| | | MTPN, EIF3F, STX16, STK38L, |
| | | TRAF4, FGD3, ARL14EP, ITK, |
| | | EPB41, MMADHC, FDPS, |
| | | SMG1, UBE2L3, PTPN11, EI24, |
| | | NUP62, PSMC4, PPID, ARF3, |
| | | MLX, METTL10, BABAM1, |
| | | CPNE3, HNRNPA1L2, SRSF1, |
| | | NFKB2, SKAP1, COPB2, CASP3, |
| | | USP10, UBE2N, LAP3, EIF5AL1, |
| | | AAMP, KPNA3, KIAA1191, |
| | | SNX10, THOC1, C1D, VPS29, |
| | | GLRX3, NXT1, USP8, CRLF3, |
| | | FKBP4, RNH1, PPP3R1, HPRT1, |
| | | NUFIP2, RGS10, SRRT, CIR1, |
| | | CCDC124, CD2BP2, UFM1, |
| | | REXO2, NFAT5, APEX1, USP33, |
| | | CHD3, GPS1, HCLS1, ACLY, |
| | | COTL1, ETF1, SMC2, SLBP, |
| | | IST1, CENPV, SSNA1, TEX10, |
| | | DNM2 |
| GO: 0005634~nucleus | 122 | DTYMK, RBM6, FOXO1, |
| | | TCEAL8, SRP19, CNOT7, MAF1, |
| | | KLHL7, KDM1A, TOP1, |
| | | GTF2E2, CSNK2A1, NUDCD1, |
| | | RAE1, DNAJC9, PTBP3, EIF1, |
| | | PPP4C, SUPT5H, IFRD2, |
| | | DDX39A, BCL10, RCOR1, |
| | | KRT10, TOPBP1, SUZ12, |
| | | ADRM1, DCUN1D1, SMARCE1, |
| | | EIF2S1, PABPC4, NOC3L, |
| | | UBE2V2, PXK, BANF1, TTC3, |
| | | VPS72, PSMB4, POLE4, PSMB7, |
| | | DDX3X, MTPN, HTATSF1, |
| | | ASF1A, TRAF4, TFDP1, EPB41, |
| | | FIBP, TSR2, YTHDC1, SMG1, |
| | | TKT, UBE2L3, RBMX, GCFC2, |
| | | PTPN11, PSMC4, PPID, MLX, |
| | | WDR4, BABAM1, RBMXL1, |
| | | DNMT1, CPNE3, SNRNP25, |
| | | KAT6A, SRSF1, UTP18, TCOF1, |
| | | NFKB2, SKAP1, CASP3, |
| | | MAPKAP1, USP10, FTSJ3, |
| | | NCOA7, CCNC, RBBP7, MCM4, |
| | | MCM5, PURA, PRPF6, NOC2L, |
| | | UBE2N, LAP3, NCOA4, KPNA3, |
| | | SNX10, THOC1, C1D, GLRX3, |
| | | POLR2K, FKBP3, NUFIP2, |
| | | RGS10, CIR1, CD2BP2, UFM1, |
| | | REXO2, DHX15, NFAT5, |
| | | TCEA1, APEX1, TRIP12, CHD3, |
| | | NUB1, HCLS1, LMNA, PHF11, |
| | | COTL1, ETF1, SMC2, SMC3, |
| | | RSBN1, SLBP, PWP1, PSMD14, |
| | | SP3, CENPV, TCEB3, SSNA1, |
| | | DNM2 |
| Chaperone | 15 | FKBP4, TIMM10, DNAJB14, |
| | | CNPY3, MESDC2, TMEM126B, |
| | | RBBP7, COTL1, HYOU1, |
| | | PSMG4, DNAJC9, PPID, TIMM9, |
| | | PDRG1, ASF1A |
| Ribonucleoprotein | 18 | HNRNPA1L2, MRPS35, |
| | | MRPS26, MRPL4, MRPS33, |
| | | RPL35, RPL39, SRP19, RBMX, |
| | | RPS19BP1, SLBP, MRPL22, |
| | | MRPL13, MRPL28, MRPL54, |
| | | RBMXL1, MRPL48, SNRPG |
| GO: 0005730~nucleolus | 34 | MEAF6, UTP18, NOC3L, |
| | | TCOF1, RPL35, PRKDC, SRP19, |
| | | MAF1, RPS19BP1, TTC3, |
| | | KLHL7, TOP1, WDR36, RAE1, |
| | | REXO2, DHX15, TCEA1, |
| | | APEX1, FTSJ3, CHD3, NUB1, |
| | | CCDC137, ITPR3, SMC2, |
| | | GCFC2, PWP1, NOC2L, SUZ12, |
| | | UBE2N, PPID, UTP14A, TEX10, |
| | | KAT6A, C1D |
| GO: 0003723~RNA binding | 27 | HNRNPA1L2, SRSF1, PABPC4, |
| | | RBM6, CNOT7, RPL39, NUFIP2, |
| | | DDX3X, RAE1, HTATSF1, |
| | | PTBP3, PRPF40A, MRPL4, |
| | | YTHDC1, THUMPD2, ETF1, |
| | | RBBP7, RBMX, PRPF6, PURA, |
| | | SUZ12, SMARCE1, DNMT1, |
| | | RBMXL1, THOC1, C1D, SNRPG |
| GO: 0005829~cytosol | 83 | TGOLN2, CAPZA2, DTYMK, |
| | | FOXO1, NFKB2, SRP19, MAF1, |
| | | CNOT7, SKAP1, COPB2, CASP3, |
| | | WDR73, CSNK2A1, S1PR1, |
| | | MAPKAP1, ABHD10, STX6, |
| | | BCL10, AARS, WNK1, POLR1C, |
| | | MFN2, UBE2N, DOK2, RFK, |
| | | EIF2S1, HARS, ADSL, VPS26B, |
| | | KPNA3, SNRPG, VPS29, NXT1, |
| | | USP8, GNAI2, POLR2K, FKBP4, |
| | | PPP3R1, NOB1, RPL35, UBA6, |
| | | CTPS1, PRKDC, ASNS, HPRT1, |
| | | VARS, RPL39, PTRH2, BANF1, |
| | | PSMB4, RGS10, PSMB7, |
| | | AKR1A1, MTPN, EIF3F, STX16, |
| | | TSTA3, EXOC2, TRIP12, FGD3, |
| | | ITK, MMADHC, LMNA, FDPS, |
| | | AK2, SMG1, TKT, ACLY, |
| | | ITPR3, ETF1, SMC2, SMC3, |
| | | PTPN11, SLBP, PSMD14, |
| | | PSMC4, IST1, WDR4, CPNE3, |
| | | PSAT1, SSNA1, EIF4E2, DNM2 |
| GO: 0000398~mRNA | 15 | SRSF1, DDX39A, PRPF4B, |
| splicing, via spliceosome | | POLR2K, CWC27, RBMX, |
| | | PRPF6, SRRT, CD2BP2, |
| | | HTATSF1, CDC40, DHX15, |
| | | SNRNP25, SNRPG, PRPF40A |
| Initiation factor | 8 | EIF2S1, EIF1AY, EIF3F, EIF1, |
| | | DENR, MCTS1, MTIF3, EIF4E2 |
| GO: 0003743~translation | 8 | EIF2S1, EIF1AY, EIF3F, EIF1, |
| initiation factor activity | | DENR, MCTS1, MTIF3, EIF4E2 |
| GO: 0070125~mitochondrial | 9 | MRPS35, MRPS26, MRPL22, |
| translational elongation | | MRPL4, MRPL13, MRPS33, |
| | | MRPL28, MRPL54, MRPL48 |
| mutagenesis site | 55 | SRSF1, TGOLN2, FOXO1, UFC1, |
| | | RAB1B, NFKB2, SKAP1, |
| | | KDM1A, TOP1, S1PR1, USP10, |
| | | SUPT5H, PPP4C, BCL10, |
| | | NCOA7, UBE2N, MFN2, |
| | | PPM1K, NCOA4, EIF5AL1, |
| | | MCTS1, THOC1, CPSF3L, |
| | | VPS29, NXT1, PRKDC, UBA6, |
| | | ASNS, PXK, BANF1, DDX3X, |
| | | AKR1A1, UFM1, HTATSF1, |
| | | REXO2, ASF1A, STK38L, |
| | | USP33, APEX1, GCDH, EPB41, |
| | | NUB1, LMNA, SMG1, COTL1, |
| | | SLBP, PTPN11, SOD2, CUL4A, |
| | | SP3, IST1, CENPV, DNMT1, |
| | | EIF4E2, KAT6A |
| GO: 0070126~mitochondrial | 9 | MRPS35, MRPS26, MRPL22, |
| translational termination | | MRPL4, MRPL13, MRPS33, |
| | | MRPL28, MRPL54, MRPL48 |
| Ribosomal protein | 12 | MRPS35, MRPS26, MRPL22, |
| | | MRPL4, MRPL13, MRPS33, |
| | | MRPL28, MRPL54, RPL35, |
| | | MRPL48, RPL39, RPS19BP1 |
| transit peptide: | 20 | GCDH, MRPS35, MRPS26, |
| Mitochondrion | | OXA1L, MMADHC, PTRH2, |
| | | MTIF3, HADHA, SOD2, |
| | | NDUFS6, MRPL22, MRPL28, |
| | | PPM1K, CLPP, REXO2, |
| | | MRPL54, ATP5C1, ABHD10, |
| | | YRDC, MRPL48 |
| RNA-binding | 24 | HNRNPA1L2, DDX39A, SRSF1, |
| | | PABPC4, AARS, YTHDC1, |
| | | RBM6, THUMPD2, CNOT7, |
| | | SRP19, RBMX, NUFIP2, SLBP, |
| | | DDX3X, EIF5AL1, HTATSF1, |
| | | EIF2S1, RBMXL1, PTBP3, |
| | | APEX1, EIF4E2, THOC1, C1D, |
| | | SNRPG |
| Transit peptide | 21 | GCDH, MRPS35, MRPS26, |
| | | OXA1L, MMADHC, STOML2, |
| | | PTRH2, MTIF3, HADHA, SOD2, |
| | | NDUFS6, MRPL22, MRPL28, |
| | | PPM1K, REXO2, CLPP, |
| | | MRPL54, ATP5C1, ABHD10, |
| | | YRDC, MRPL48 |
| GO: 0008380~RNA splicing | 12 | HNRNPA1L2, CIR1, PRPF4B, |
| | | CDC40, DHX15, RBMXL1, |
| | | PTBP3, SREK1IP1, SNRNP25, |
| | | THOC1, PRPF6, SNRPG |
| Protein transport | 22 | VPS29, NXT1, STX6, SNX9, |
| | | KDELR2, TIMM10, RAB1B, |
| | | TOMM40, ARF5, LMAN2, |
| | | COPB2, NUP62, PPID, EIF5AL1, |
| | | ARF3, STX16, TIMM9, TMED10, |
| | | VPS26B, KPNA3, SNX10, |
| | | EXOC2 |
| GO: 0006406~mRNA | 9 | NXT1, SRSF1, DDX39A, NUP62, |
| export from nucleus | | RAE1, CDC40, SMG1, SLBP, |
| | | THOC1 |
| GO: 0016607~nuclear | 12 | NXT1, SRSF1, CIR1, DDX3X, |
| speck | | CD2BP2, NOC3L, LMNA, |
| | | YTHDC1, APEX1, THOC1, |
| | | PRPF6, PRPF40A |
| Coiled coil | 65 | MRPS35, COA3, TCOF1, RBM6, |
| | | STOML2, TCEAL8, NFKB2, |
| | | THADA, COPB2, KDM1A, |
| | | TOP1, ATAD3B, DNAJC9, |
| | | SREK1IP1, SUPT5H, FTSJ3, |
| | | STX6, RCOR1, WNK1, CNPY3, |
| | | CCDC47, NCOA7, KRT10, |
| | | CCDC137, PURA, MFN2, |
| | | SMARCE1, EIF2S1, HARS, |
| | | MEAF6, USP8, CRLF3, FKBP4, |
| | | NOC3L, ARF5, VARS, TTC3, |
| | | SRRT, FAM107B, CCDC124, |
| | | STX16, EXOC2, TRAF4, CHD3, |
| | | SMCHD1, CCDC25, NUB1, |
| | | CWC27, HCLS1, TMEM120B, |
| | | LMNA, SMG1, YTHDC1, |
| | | TPD52L2, ITPR3, SMC2, GCFC2, |
| | | SMC3, NUP62, PSMC4, IST1, |
| | | MLX, UTP14A, SSNA1, |
| | | GPATCH8 |
| GO: 0003682~chromatin | 17 | NOC3L, FOXO1, NFKB2, |
| binding | | RBMX, SMC3, MCM5, NOC2L, |
| | | KDM1A, TOP1, SMARCE1, |
| | | NUP62, SP3, CKS2, DNMT1, |
| | | SUPT5H, ASF1A, TEX10 |
| Repressor | 20 | RCOR1, CCNC, NFKB2, MAF1, |
| | | CNOT7, RBBP7, RBMX, GCFC2, |
| | | NOC2L, SUZ12, KDM1A, CIR1, |
| | | SP3, MLX, DNMT1, PTBP3, |
| | | SUPT5H, APEX1, C1D, KAT6A |
| hsa03040: Spliceosome | 10 | HNRNPA1L2, SRSF1, CDC40, |
| | | DHX15, RBMXL1, RBMX, |
| | | THOC1, PRPF6, PRPF40A, |
| | | SNRPG |
| GO: 0032784~regulation of | 4 | HTATSF1, TCEA1, SUPT5H, |
| DNA-templated transcription, | | THOC1 |
| elongation | | |
| hsa03008: Ribosome | 8 | NXT1, WDR36, CSNK2A1, |
| biogenesis in eukalyotes | | UTP18, REXO2, TCOF1, NOB1, |
| | | UTP14A |
| Mitochondrion inner | 12 | NDUFS6, NDUFA2, OXA1L, |
| membrane | | NDUFB6, ATAD3B, COA3, |
| | | TIMM9, TIMM10, ATP5C1, |
| | | STOML2, SLC25A39, HIGD2A |
| GO: 0006270~DNA | 5 | PRIM1, TOPBP1, MCM4, |
| replication initiation | | MCM5, PURA |
| Neuropathy | 7 | MFN2, AARS, LMNA, HARS, |
| | | WNK1, DNMT1, DNM2 |
| GO: 0006890~retrograde | 7 | COPB2, KDELR2, ARF3, |
| vesicle-mediated transport, | | TMED10, RAB1B, LMAN2, |
| Golgi to ER | | ARF5 |
| GO: 0006405~RNA export | 6 | NXT1, SRSF1, DDX39A, NUP62, |
| from nucleus | | CDC40, THOC1 |
| Spliceosome | 8 | HNRNPA1L2, SRSF1, PRPF4B, |
| | | CDC40, RBMX, SNRNP25, |
| | | PRPF6, SNRPG |
| SM00968: SM00968 | 3 | SMCHD1, SMC2, SMC3 |
| GO: 0006368~transcription | 7 | TAF11, ADRM1, GTF2E2, |
| elongation from RNA | | POLR2K, TCEB3, TCEA1, |
| polymerase II promoter | | SUPT5H |
| Chromatin regulator | 12 | SUZ12, KDM1A, MEAF6, |
| | | SMARCE1, RCOR1, BABAM1, |
| | | DNMT1, RBBP7, ASF1A, VPS72, |
| | | CHD3, KAT6A |
| GO: 0071013~catalytic | 7 | SRSF1, PRPF4B, CWC27, |
| step 2 spliceosome | | CDC40, RBMX, PRPF6, SNRPG |
| GO: 0006511~ubiquitin- | 10 | UBE2N, PSMD14, USP8, |
| dependent protein catabolic | | CUL4A, NUB1, UBA6, USP10, |
| process | | UBE2L3, USP33, TTC3 |
| DNA repair | 12 | UBE2N, PSMD14, CUL4A, |
| | | BABAM1, SMG1, PRKDC, |
| | | TOPBP1, USP10, APEX1, SMC3, |
| | | TRIP12, BOD1L1 |
| GO: 0050852~T cell | 9 | UBE2N, ITK, BCL10, PSMB4, |
| receptor signaling pathway | | PSMD14, PSMB7, PSMC4, |
| | | STOML2, SKAP1 |
| IPR010935: SMCs flexible | 3 | SMCHD1, SMC2, SMC3 |
| hinge | | |
| GO: 0006369~termination | 6 | SRSF1, DDX39A, CDC40, SLBP, |
| of RNA polymerase II | | THOC1, SNRPG |
| transcription | | |
| GO: 0000784~nuclear | 8 | KDM1A, SMCHD1, PRKDC, |
| chromosome, telomeric | | APEX1, MCM4, MCM5, THOC1, |
| region | | PURA |
| DNA damage | 13 | PRKDC, SMG1, TOPBP1, SMC3, |
| | | BOD1L1, UBE2N, PSMD14, |
| | | CUL4A, BABAM1, USP10, |
| | | APEX1, MCTS1, TRIP12 |
| GO: 1901796~regulation of | 8 | TAF11, MEAF6, CSNK2A1, |
| signal transduction by p53 | | TOPBP1, RBBP7, CHD3, |
| class mediator | | KAT6A, NOC2L |
| GO: 0038061~NIK/NF- | 6 | PSMB4, PSMD14, PSMB7, |
| kappaB signaling | | PSMC4, NFKB2, PPP4C |
| Nucleotide-binding | 40 | PRPF4B, GNAI2, DTYMK, |
| | | CTPS1, RAB1B, UBA6, PRKDC, |
| | | ASNS, ARF5, VARS, HPRT1, |
| | | ATAD3B, CSNK2A1, DDX3X, |
| | | DHX15, STK38L, CHD3, |
| | | DDX39A, ITK, AARS, ATP11B, |
| | | WNK1, AK2, SMG1, ACLY, |
| | | ARL16, UBE2L3, MCM4, SMC2, |
| | | MCM5, SMC3, MFN2, UBE2N, |
| | | HYOU1, PSMC4, RFK, ARF3, |
| | | HARS, HSPA13, DNM2 |
| GO: 0043234~protein | 15 | BCL10, OXA1L, EPB41, FKBP4, |
| complex | | PXK, VPS72, PTPN11, UBE2N, |
| | | KDM1A, SMARCE1, NUP62, |
| | | DNMT1, USP10, ASF1A, DNM2 |
| Translation, ribosomal | 6 | CPSF3L, METTL5, EIF2S1, |
| structure and biogenesis | | HARS, MCTS1, MTIF3 |
| GO: 0005694~chromosome | 7 | TOP1, PRPF4B, SMCHD1, |
| | | CCDC137, TOPBP1, SMC3, |
| | | BOD1L1 |
| Charcot-Marie-Tooth | 5 | MFN2, AARS, LMNA, HARS, |
| disease | | DNM2 |
| GO: 0005758~mitochondrial | 6 | DTYMK, REXO2, TIMM9, |
| intermembrane space | | TIMM10, AK2, STOML2 |
| GO: 0070536~protein | 4 | PSMD14, USP8, BABAM1, |
| K63-linked deubiquitination | | USP33 |
| Activator | 19 | MEAF6, FOXO1, NCOA7, |
| | | PHF11, CCNC, NFKB2, RBMX, |
| | | PURA, SRRT, NCOA4, SP3, |
| | | HTATSF1, MLX, NFAT5, |
| | | DNMT1, SUPT5H, APEX1, |
| | | KAT6A, TFDP1 |
| Proteasome | 5 | PSMB4, ADRM1, PSMD14, |
| | | PSMB7, PSMC4 |
| GO: 0032790~ribosome | 3 | DENR, MCTS1, MTIF3 |
| disassembly | | |
| hsa03013: RNA transport | 10 | NXT1, NUP62, RAE1, EIF2S1, |
| | | EIF1AY, PABPC4, EIF3F, EIF1, |
| | | EIF4E2, THOC1 |
| GO: 0043130~ubiquitin | 6 | UBE2N, BCL10, NUP62, RAE1, |
| binding | | CKS2, USP33 |
| GO: 0043022~ribosome | 5 | EIF5AL1, EIF2S1, SPCS1, ETF1, |
| binding | | MTIF3 |
| Isomerase | 7 | TOP1, FKBP4, CWC27, PPID, |
| | | FKBP3, TOPBP1, TSTA3 |
| Ubl conjugation pathway | 19 | USP8, UFC1, UBA6, UBE2V2, |
| | | UBE2L3, TTC3, UBE2N, KLHL7, |
| | | DCUN1D1, PSMD14, CUL4A, |
| | | UFM1, EIF3F, BABAM1, DDA1, |
| | | USP10, USP33, ALG13, TRIP12 |
| Elongation factor | 4 | EIF5AL1, TCEB3, TCEA1, |
| | | SUPT5H |
| ATP-binding | 32 | PRPF4B, DTYMK, CTPS1, |
| | | UBA6, PRKDC, ASNS, VARS, |
| | | ATAD3B, CSNK2A1, DDX3X, |
| | | DHX15, STK38L, CHD3, |
| | | DDX39A, ITK, AARS, ATP11B, |
| | | WNK1, SMG1, AK2, ACLY, |
| | | UBE2L3, SMC2, MCM4, MCM5, |
| | | SMC3, UBE2N, HYOU1, PSMC4, |
| | | RFK, HARS, HSPA13 |
| Cell division | 13 | SNX9, ATAD3B, GNAI2, |
| | | CCDC124, IST1, CDC40, CKS2, |
| | | CENPV, BABAM1, SMC2, |
| | | SMC3, MCM5, PRPF40A |
| SM00320: WD40 | 10 | COPB2, WDR36, WDR73, |
| | | UTP18, RAE1, CDC40, AAMP, |
| | | WDR4, RBBP7, PWP1 |
| hsa01130: Biosynthesis of | 11 | GCDH, HSD17B10, PYCR1, |
| antibiotics | | AKR1A1, FDPS, ADSL, AK2, |
| | | ACLY, TKT, PSAT1, HADHA |
| GO: 0006457~protein | 9 | CSNK2A1, GNAI2, FKBP4, |
| folding | | CWC27, PPID, ERP29, AARS, |
| | | MESDC2, PDRG1 |
| hsa03060: Protein export | 4 | OXA1L, SEC11A, SPCS1, SRP19 |
| ER-Golgi transport | 6 | COPB2, KDELR2, ARF3, |
| | | TMED10, ARF5, ERGIC3 |
| Cell cycle | 18 | SNX9, USP8, GNAI2, SMC2, |
| | | MCM4, SMC3, MCM5, |
| | | ATAD3B, CSNK2A1, CCDC124, |
| | | IST1, CDC40, BABAM1, |
| | | CENPV, CKS2, MCTS1, TFDP1, |
| | | PRPF40A |
| GO: 0045739~positive | 4 | UBE2N, BABAM1, UBE2V2, |
| regulation of DNA repair | | APEX1 |
| GO: 0050699~WW domain | 4 | NDFIP2, TCEAL8, TRAF4, |
| binding | | DNM2 |
| GO: 0000502~proteasome | 5 | PSMB4, ADRM1, PSMD14, |
| complex | | PSMB7, PSMC4 |
| GO: 0005794~Golgi | 23 | TGOLN2, STX6, KDELR2, |
| apparatus | | USP8, ATP11B, NDFIP2, |
| | | RAB1B, TOPBP1, ARF5, |
| | | LMAN2, ERGIC3, PWP1, TAF11, |
| | | EI24, SP3, ARF3, MAPKAP1, |
| | | STX16, TMED10, USP33, FGD3, |
| | | DNM2, KAT6A |
| GO: 0038095~Fc-epsilon | 8 | UBE2N, ITK, BCL10, PSMB4, |
| receptor signaling pathway | | PSMD14, PSMB7, PSMC4, |
| | | PPP3R1 |
| GO: 0005840~ribosome | 8 | MRPL4, MRPL13, MRPS33, |
| | | EIF2S1, MRPL54, RPL35, |
| | | APEX1, RPS19BP1 |
| Rotamase | 4 | FKBP4, CWC27, PPID, FKBP3 |
| GO: 0030133~transport | 6 | TGOLN2, COPB2, KDELR2, |
| vesicle | | ERP29, TMED10, RAB1B |
| hsa00240: Pyrimidine | 7 | PRIM1, POLE4, POLR2K, |
| metabolism | | DTYMK, CTPS1, POLR1C, |
| | | ENTPD4 |
| repeat: WD 3 | 10 | COPB2, WDR36, WDR73, |
| | | UTP18, RAE1, CDC40, AAMP, |
| | | WDR4, RBBP7, PWP1 |
| GO: 0010494~cytoplasmic | 4 | DDX3X, EIF2S1, PABPC4, |
| stress granule | | NUFIP2 |
| GO: 0003746~translation | 4 | EIF5AL1, TCEB3, TCEA1, |
| elongation factor activity | | SUPT5H |
| GO: 0006303~double-strand | 5 | UBE2N, PSMD14, BABAM1, |
| break repair via | | PRKDC, UBE2V2 |
| nonhomologous end joining | | |
| GO: 0006626~protein | 4 | MFN2, TIMM9, TIMM10, |
| targeting to mitochondrion | | TOMM40 |
| GO: 0032981~mitochondrial | 5 | NDUFS6, NDUFA2, OXA1L, |
| respiratory chain complex I | | NDUFB6, TMEM126B |
| assembly | | |
| WD repeat | 10 | COPB2, WDR36, WDR73, |
| | | UTP18, RAE1, CDC40, AAMP, |
| | | WDR4, RBBP7, PWP1 |
| active site: Glycyl thioester | 5 | UBE2N, UFC1, UBA6, UBE2L3, |
| intermediate | | TRIP12 |
| GO: 0061077~chaperone- | 4 | CSNK2A1, FKBP4, PPID, FKBP3 |
| mediated protein folding | | |
| GO: 0003713~transcription | 10 | TAF11, BCL10, SMARCE1, |
| coactivator activity | | NCOA4, NFKB2, UBE2L3, |
| | | APEX1, PRPF6, KAT6A, TFDP1 |
| repeat: WD 1 | 10 | COPB2, WDR36, WDR73, |
| | | UTP18, RAE1, CDC40, AAMP, |
| | | WDR4, RBBP7, PWP1 |
| repeat: WD 2 | 10 | COPB2, WDR36, WDR73, |
| | | UTP18, RAE1, CDC40, AAMP, |
| | | WDR4, RBBP7, PWP1 |
| Chromosomal rearrangement | 11 | SUZ12, HSD17B10, BCL10, |
| | | TOP1, MEAF6, NCOA4, FOXO1, |
| | | TCEA1, NFKB2, THADA, |
| | | KAT6A |
| GO: 0006974~cellular | 9 | DDX39A, CASP3, CUL4A, |
| response to DNA damage | | FOXO1, TOPBP1, USP10, |
| stimulus | | MCTS1, TRIP12, BOD1L1 |
| IPR001680: WD40 repeat | 10 | COPB2, WDR36, WDR73, |
| | | UTP18, RAE1, CDC40, AAMP, |
| | | WDR4, RBBP7, PWP1 |
| SM00360: RRM | 8 | HNRNPA1L2, SRSF1, HTATSF1, |
| | | PABPC4, RBMXL1, RBM6, |
| | | PTBP3, RBMX |
| GO: 0005643~nuclear pore | 5 | NXT1, NUP62, RAE1, EIF5AL1, |
| | | KPNA3 |
| GO: 0006412~translation | 10 | MRPL22, MRPL4, MRPL13, |
| | | MRPS33, MRPL28, PABPC4, |
| | | HARS, RPL35, SLC25A39, |
| | | RPL39 |
| GO: 0006450~regulation of | 3 | AARS, YRDC, VARS |
| translational fidelity | | |
| GO: 0004407~histone | 4 | KDM1A, RCOR1, RBBP7, CHD3 |
| deacetylase activity | | |
| GO: 0006413~translational | 7 | EIF2S1, EIF1AY, EIF3F, RPL35, |
| initiation | | EIF1, RPL39, EIF4E2 |
| GO: 0051301~cell division | 12 | ATAD3B, GNAI2, CCDC124, |
| | | IST1, CDC40, CKS2, CENPV, |
| | | BABAM1, SMC2, MCM5, SMC3, |
| | | PRPF40A |
| GO: 0042795~snRNA | 5 | TAF11, CPSF3L, SRRT, GTF2E2, |
| transcription from RNA | | POLR2K |
| polymerase II promoter | | |
| GO: 0005682~U5 snRNP | 3 | CD2BP2, PRPF6, SNRPG |
| GO: 0071004~U2-type | 3 | PRPF39, PRPF40A, SNRPG |
| prespliceosome | | |
| GO: 0002223~stimulatory | 6 | UBE2N, BCL10, PSMB4, |
| C-type lectin receptor | | PSMD14, PSMB7, PSMC4 |
| signaling pathway | | |
| GO: 0031072~heat shock | 4 | FKBP4, DNAJC9, PPID, LMAN2 |
| protein binding | | |
| GO: 0005759~mitochondrial | 11 | GCDH, HSD17B10, PYCR1, |
| matrix | | PPM1K, DTYMK, REXO2, |
| | | CLPP, ATP5C1, ABHD10, BCO2, |
| | | SOD2 |
| GO: 0008565~protein | 5 | VPS29, TIMM9, TIMM10, |
| transporter activity | | KPNA3, VPS26B |
| GO: 0031372~UBC13- | 2 | UBE2N, UBE2V2 |
| MMS2 complex | | |
| GO: 0042101~T cell receptor | 3 | BCL10, STOML2, SKAP1 |
| complex | | |
| GO: 0016581~NuRD | 3 | CSNK2A1, RBBP7, CHD3 |
| complex | | |
| GO: 0006376~mRNA splice | 3 | SRSF1, YTHDC1, RBMXL1 |
| site selection | | |
| GO: 0051262~protein | 4 | OXA1L, ADSL, CCL5, UXS1 |
| tetramerization | | |
| GO: 0005524~ATP binding | 35 | PRPF4B, FKBP4, DTYMK, |
| | | CTPS1, UBA6, PRKDC, ASNS, |
| | | PXK, VARS, ATAD3B, |
| | | CSNK2A1, DDX3X, DHX15, |
| | | STK38L, CHD3, DDX39A, ITK, |
| | | SMCHD1, AARS, ATP11B, |
| | | WNK1, SMG1, AK2, ACLY, |
| | | UBE2L3, SMC2, MCM4, MCM5, |
| | | SMC3, UBE2N, HYOU1, |
| | | PSMC4, RFK, HARS, HSPA13 |
| GO: 0003735~structural | 9 | MRPS35, MRPL22, MRPL4, |
| constituent of ribosome | | MRPL13, MRPS33, MRPL28, |
| | | RPL35, SLC25A39, RPL39 |
| Transcription | 46 | MEAF6, POLR2K, FOXO1, |
| | | TCEAL8, NFKB2, MAF1, |
| | | CNOT7, VPS72, PRIM1, SRRT, |
| | | KDM1A, GTF2E2, CSNK2A1, |
| | | CIR1, DDX3X, HTATSF1, |
| | | GTF3C6, NFAT5, TCEA1, |
| | | SUPT5H, ASF1A, APEX1, |
| | | TFDP1, CHD3, RCOR1, NCOA7, |
| | | POLR1C, CCNC, PHF11, RBBP7, |
| | | UBE2L3, RBMX, GCFC2, PURA, |
| | | NOC2L, TAF11, SUZ12, NCOA4, |
| | | MLX, SP3, TCEB3, DNMT1, |
| | | MCTS1, THOC1, KAT6A, C1D |
| GO: 0000413~protein | 4 | FKBP4, CWC27, PPID, FKBP3 |
| peptidyl-prolyl isomerization | | |
| repeat: WD 4 | 9 | COPB2, WDR36, UTP18, RAE1, |
| | | CDC40, AAMP, WDR4, RBBP7, |
| | | PWP1 |
| GO: 0005685~U1 snRNP | 3 | PRPF39, PRPF40A, SNRPG |
| hsa03030: DNA replication | 4 | PRIM1, POLE4, MCM4, MCM5 |
| rRNA processing | 5 | WDR36, TSR2, UTP18, FTSJ3, |
| | | C1D |
| GO: 0007077~mitotic | 4 | NUP62, RAE1, LMNA, BANF1 |
| nuclear envelope disassembly | | |
| IPR012340: Nucleic acid- | 5 | EIF5AL1, EIF2S1, EIF1AY, |
| binding, OB-fold | | MCM4, MCM5 |
| GO: 0048037~cofactor | 3 | ACLY, TKT, ASNS |
| binding | | |
| compositionally biased | 8 | HTATSF1, TCOF1, MRPL48, |
| region: Poly-Lys | | TTC3, RSBN1, THADA, CHD3, |
| | | KAT6A |
| GO: 0006886~intracellular | 9 | STX6, VPS29, SNX9, COPB2, |
| protein transport | | KDELR2, STX16, ERP29, |
| | | TMED10, VPS26B |
| GO: 0005545~1- | 3 | SNX9, EPB41, SNX10 |
| phosphatidylinositol binding | | |
| IPR019775: WD40 repeat, | 7 | WDR36, UTP18, RAE1, CDC40, |
| conserved site | | AAMP, RBBP7, PWP1 |
| GO: 0003755~peptidyl-prolyl | 4 | FKBP4, CWC27, PPID, FKBP3 |
| cis-trans isomerase activity | | |
| GO: 0031625~ubiquitin | 10 | MFN2, UBE2N, BCL10, SNX9, |
| protein ligase binding | | CUL4A, FOXO1, UBE2V2, |
| | | UBE2L3, EIF4E2, TRAF4 |
| GO: 0008134~transcription | 10 | KDM1A, BCL10, DDX3X, |
| factor binding | | RCOR1, PPID, MLX, PRKDC, |
| | | KAT6A, TFDP1, PURA |
| GO: 0071008~U2-type | 2 | DHX15, GCFC2 |
| post-mRNA release | | |
| spliceosomal complex | | |
| GO: 1900087~positive | 3 | DDX3X, CUL4A, APEX1 |
| regulation of G1/S | | |
| transition of mitotic cell | | |
| cycle | | |
| SM00361: RRM_1 | 3 | PABPC4, RBMXL1, RBMX |
| IPR017986: WD40-repeat- | 10 | COPB2, WDR36, WDR73, |
| containing domain | | UTP18, RAE1, CDC40, AAMP, |
| | | WDR4, RBBP7, PWP1 |
| GO: 0031124~mRNA | 4 | SRSF1, DDX39A, CDC40, |
| 3′-end processing | | THOC1 |
| Protease | 14 | LAP3, PSMB4, PSMD14, CASP3, |
| | | PSMB7, USP8, SEC11A, EIF3F, |
| | | CLPP, HMCES, SPCS1, USP10, |
| | | USP33, ALG13 |
| GO: 0051443~positive | 3 | UBE2N, DCUN1D1, UBE2L3 |
| regulation of ubiquitin- | | |
| protein transferase activity | | |
| GO: 0006521~regulation of | 4 | PSMB4, PSMD14, PSMB7, |
| cellular amino acid | | PSMC4 |
| metabolic process | | |
| GO: 0032481~positive | 4 | POLR2K, PRKDC, POLR1C, |
| regulation of type I | | NFKB2 |
| interferon production | | |
| hsa05010: Alzheimer's | 8 | HSD17B10, NDUFS6, NDUFA2, |
| disease | | CASP3, NDUFB6, PPP3R1, |
| | | ATP5C1, ITPR3 |
| Amino-acid biosynthesis | 3 | PYCR1, ASNS, PSAT1 |
| GO: 0017053~transcriptional | 4 | SMARCE1, RCOR1, SP3, C1D |
| repressor complex | | |
| Hydrolase | 33 | CPSF3L, USP8, PTRH2, CNOT7, |
| | | PSMB4, CASP3, PSMB7, |
| | | DDX3X, REXO2, EIF3F, DHX15, |
| | | ABHD10, USP10, ENTPD4, |
| | | PPP4C, USP33, APEX1, CHD3, |
| | | DDX39A, SEC11A, HMCES, |
| | | ATP11B, MCM4, MCM5, |
| | | PTPN11, LAP3, MFN2, PSMD14, |
| | | PPM1K, CLPP, SPCS1, ALG13, |
| | | DNM2 |
| IPR012677: Nucleotide- | 9 | HNRNPA1L2, SRSF1, SRRT, |
| binding, alpha-beta plait | | HTATSF1, PABPC4, RBMXL1, |
| | | RBM6, PTBP3, RBMX |
| GO: 0001731~formation of | 3 | EIF3F, DENR, MCTS1 |
| translation preinitiation | | |
| complex | | |
| Helicase | 6 | DDX39A, DDX3X, DHX15, |
| | | MCM4, MCM5, CHD3 |
| GO: 0005802~trans-Golgi | 6 | TGOLN2, STX6, LAP3, SNX9, |
| network | | STX16, DNM2 |
| IPR016135: Ubiquitin- | 4 | UBE2N, UFC1, UBE2V2, |
| conjugating enzyme/RWD- | | UBE2L3 |
| like | | |
| GO: 0006357~regulation of | 13 | TCEAL8, NFKB2, PURA, SOD2, |
| transcription from RNA | | KDM1A, CIR1, SMARCE1, |
| polymerase II promoter | | HTATSF1, TCEB3, TCEA1, |
| | | SUPT5H, CHD3, TFDP1 |
| GO: 0032403~protein | 8 | CASP3, IST1, HCLS1, TMED10, |
| complex binding | | APEX1, SKAP1, HADHA, DNM2 |
| GO: 0005763~mitochondrial | 3 | MRPS35, MRPS26, MRPS33 |
| small ribosomal subunit | | |
| hsa03050: Proteasome | 4 | PSMB4, PSMD14, PSMB7, |
| | | PSMC4 |
| GO: 0005681~spliceosomal | 5 | HNRNPA1L2, DDX39A, CDC40, |
| complex | | PRPF6, SNRPG |
| GO: 0000166~nucleotide | 11 | HNRNPA1L2, SRSF1, SRRT, |
| binding | | HTATSF1, PABPC4, RBMXL1, |
| | | RBM6, PTBP3, PXK, HPRT1, |
| | | RBMX |
| Proto-oncogene | 8 | SUZ12, TOP1, DCUN1D1, |
| | | CSNK2A1, NCOA4, FOXO1, |
| | | NFKB2, KAT6A |
| IPR016040: NAD(P)-binding | 7 | HSD17B10, PYCR1, UBA6, |
| domain | | ACLY, TSTA3, UXS1, HADHA |
| IPR024969: Rpn11/EIF3F | 2 | PSMD14, EIF3F |
| C-terminal domain | | |
| IPR001950: Translation | 2 | EIF1, DENR |
| initiation factor SUI1 | | |
| GO: 1990391~DNA repair | 2 | KDM1A, RCOR1 |
| complex | | |
| GO: 0030906~retromer, | 2 | VPS29, VPS26B |
| cargo-selective complex | | |
| IPR003954: RNA recognition | 3 | PABPC4, RBMXL1, RBMX |
| motif domain, eukaryote | | |
| GO: 0005689~U12-type | 3 | DHX15, SNRNP25, SNRPG |
| spliceosomal complex | | |
| GO: 0009055~electron carrier | 5 | GLRX3, GCDH, NDUFS6, |
| activity | | AKR1A1, TSTA3 |
| GO: 0070062~extracellular | 54 | SRSF1, CAPZA2, RAB1B, UFC1, |
| exosome | | UXS1, VPS13D, KDELR2, |
| | | ERP29, AARS, KRT10, LAP3, |
| | | UBE2N, EIF2S1, ATP5C1, |
| | | VPS29, GLRX3, SNX9, GNAI2, |
| | | FKBP4, RNH1, UBE2V2, |
| | | LMAN2, ARF5, HPRT1, BANF1, |
| | | PSMB4, PSMB7, AKR1A1, |
| | | DDX3X, UFM1, MTPN, |
| | | TMED10, TSTA3, HPCAL1, |
| | | FIBP, CCDC25, SEC11A, AK2, |
| | | TOMM40, ACLY, TKT, UBE2L3, |
| | | COTL1, SMC2, RBMX, SOD2, |
| | | HYOU1, PSMD14, IST1, ARF3, |
| | | CPNE3, HSPA13, PSAT1, DNM2 |
| Respiratory chain | 4 | NDUFS6, NDUFA2, NDUFB6, |
| | | HIGD2A |
| GO: 0015031~protein | 12 | VPS29, KDELR2, IST1, ARF3, |
| transport | | EIF5AL1, PPID, TIMM9, |
| | | RAB1B, LMAN2, ARF5, SNX10, |
| | | EXOC2 |
| IPR000504: RNA | 8 | HNRNPA1L2, SRSF1, HTATSF1, |
| recognition motif domain | | PABPC4, RBMXL1, RBM6, |
| | | PTBP3, RBMX |
| hsa00230: Purine metabolism | 8 | PRIM1, POLE4, POLR2K, ADSL, |
| | | AK2, POLR1C, ENTPD4, HPRT1 |
| Mental retardation | 9 | HSD17B10, WDR73, DDX3X, |
| | | SMARCE1, ASNS, RBMX, |
| | | SMC3, KAT6A, PURA |
| GO: 0015949~nucleobase- | 3 | DTYMK, CTPS1, AK2 |
| containing small molecule | | |
| interconversion | | |
| GO: 0046580~negative | 3 | MFN2, NUP62, MAPKAP1 |
| regulation of Ras protein | | |
| signal transduction | | |
| GO: 0030529~intracellular | 6 | HNRNPA1L2, NUP62, PABPC4, |
| ribonucleoprotein complex | | RBMXL1, RBMX, SLBP |
| hsa05012: Parkinson's | 7 | NDUFS6, NDUFA2, CASP3, |
| disease | | NDUFB6, GNAI2, ATP5C1, |
| | | UBE2L3 |
| Thiol protease | 6 | CASP3, USP8, EIF3F, USP10, |
| | | USP33, ALG13 |
| Ribosome biogenesis | 4 | WDR36, DDX3X, UTP14A, |
| | | FTSJ3 |
| GO: 0006364~rRNA | 8 | WDR36, UTP18, NOB1, RPL35, |
| processing | | RPL39, UTP14A, ILX10, C1D |
| GO: 0045070~positive | 3 | DDX3X, PPID, CCL5 |
| regulation of viral genome | | |
| replication | | |
| GO: 0000245~spliceosomal | 3 | GCFC2, PRPF6, SNRPG |
| complex assembly | | |
| mRNA transport | 5 | HNRNPA1L2, SRSF1, NUP62, |
| | | EIF5AL1, THOC1 |
| GO: 0005761~mitochondrial | 3 | MRPL13, MRPL28, MRPL48 |
| ribosome | | |
| GO: 0014823~response to | 4 | DNMT1, PRKDC, CCL5, SOD2 |
| activity | | |
| IPR015943: WD40/YVTN | 10 | COPB2, WDR36, WDR73, |
| repeat-like-containing domain | | UTP18, RAE1, CDC40, AAMP, |
| | | WDR4, RBBP7, PWP1 |
| GO: 0051879~Hsp90 protein | 3 | CSNK2A1, NUP62, PPID |
| binding | | |
| GO: 0046966~thyroid | 3 | TAF11, NUP62, TRIP12 |
| hormone receptor binding | | |
| GO: 0030687~preribosome, | 3 | AAMP, FTSJ3, TEX10 |
| large subunit precursor | | |
| IPR024704: Structural | 2 | SMC2, SMC3 |
| maintenance of chromosomes | | |
| protein | | |
| IPR001509: NAD-dependent | 2 | TSTA3, UXS1 |
| epimemse/dehydratase | | |
| GO: 0042719~mitochondrial | 2 | TIMM9, TIMM10 |
| intermembrane space protein | | |
| transporter complex | | |
| GO: 0031313~extrinsic | 2 | USP8, SNX10 |
| component of endosome | | |
| membrane | | |
| GO: 0047485~protein | 5 | TAF11, CSNK2A1, SMARCE1, |
| N-terminus binding | | BANF1, EXOC2 |
| GO: 0006397~mRNA | 7 | HNRNPA1L2, SRSF1, CIR1, |
| processing | | DHX15, RBMXL1, PTBP3, |
| | | SREK1IP1 |
| GO: 0033116~endoplasmic | 4 | TMED10, RAB1B, LMAN2, |
| reticulum-Golgi | | ERGIC3 |
| intermediate compartment | | |
| membrane | | |
| GO: 0048471~perinuclear | 16 | STX6, BCL10, FKBP4, LMNA, |
| region of cytoplasm | | NDFIP2, ARF5, TPD52L2, |
| | | MAF1, ITPR3, ARF3, MTPN, |
| | | STX16, APEX1, USP33, TRAF4, |
| | | DNM2 |
| GO: 0051881~regulation | 3 | PYCR1, BCO2, SOD2 |
| of mitochondrial membrane | | |
| potential | | |
| SM01343: SM01343 | 2 | SMG1, PRKDC |
| GO: 0004197~cysteine-type | 4 | CASP3, USP8, USP10, USP33 |
| endopeptidase activity | | |
| GO: 0002192~IRES- | 2 | DENR, MCTS1 |
| dependent translational | | |
| initiation | | |
| GO: 0071569~protein | 2 | UFM1, UFC1 |
| ufmylation | | |
| GO: 1990592~protein | 2 | UFM1, UFC1 |
| K69-linked ufmylation | | |
| GO: 0051169~nuclear | 2 | NUP62, BANF1 |
| transport | | |
| GO: 0042531~positive | 2 | HCLS1, CCL5 |
| regulation of tyrosine | | |
| phosphorylation of STAT | | |
| protein | | |
| GO: 0045039~protein | 2 | TIMM9, TIMM10 |
| import into mitochondrial | | |
| inner membrane | | |
| GO: 0008283~cell | 11 | SRRT, KDM1A, USP8, GNAI2, |
| proliferation | | DTYMK, CKS2, PRKDC, |
| | | UBE2V2, RBBP7, UBE2L3, |
| | | TFDP1 |
| GO: 0000209~protein | 7 | PSMB4, PSMD14, PSMB7, |
| polyubiquitination | | PSMC4, UBE2V2, UBE2L3, |
| | | TRIP12 |
| GO: 0061631~ubiquitin | 3 | UBE2N, UBE2V2, UBE2L3 |
| conjugating enzyme activity | | |
| GO: 0008135~translation | 3 | EIF1, MTIF3, EIF4E2 |
| factor activity, RNA binding | | |
| h_tnfr1Pathway: TNFR1 | 3 | CASP3, LMNA, PRKDC |
| Signaling Pathway | | |
| Endoplasmic reticulum | 22 | KDELR2, SEC11A, ERP29, |
| | | ATP11B, DNAJB14, CNPY3, |
| | | ALG5, MESDC2, LMAN2, |
| | | ITPR3, ERGIC3, HYOU1, EI24, |
| | | ELOVL5, EIF5AL1, TMEM170A, |
| | | TMED10, SPCS1, HSPA13, |
| | | APEX1, ALG13, SSR2 |
| IPR002909: Cell surface | 3 | NFAT5, NFKB2, EXOC2 |
| receptor IPT/TIG | | |
| GO: 0016020~membrane | 43 | GNAI2, CAPZA2, RPL35, |
| | | DNAJB14, PRKDC, CTPS1, |
| | | STOML2, ALG5, LMAN2, |
| | | CNOT7, PTRH2, NUFIP2, |
| | | PRIM1, ELOVL5, STX16, EIF3F, |
| | | YRDC, STK38L, EXOC2, |
| | | PRPF40A, DDX39A, HPCAL1, |
| | | FIBP, HCLS1, ERP29, AARS, |
| | | ATP11B, WNK1, CCDC47, |
| | | KRT10, ACLY, ITPR3, MCM4, |
| | | RBMX, MCM5, PRPF6, ERGIC3, |
| | | ADRM1, HYOU1, EI24, PSMC4, |
| | | EIF2S1, ATP5C1 |
| GO: 0002479~antigen | 4 | PSMB4, PSMD14, PSMB7, |
| processing and presentation | | PSMC4 |
| of exogenous peptide | | |
| antigen via MHC class I, | | |
| TAP-dependent | | |
| domain: FATC | 2 | SMG1, PRKDC |
| domain: FAT | 2 | SMG1, PRKDC |
| short sequence motif: Twin | 2 | TIMM9, TIMM10 |
| CX3C motif | | |
| IPR027417: P-loop | 20 | DDX39A, GNAI2, DTYMK, |
| containing nucleoside | | AK2, RAB1B, CTPS1, ARL16, |
| | | ARF5, SMC2, MCM4, SMC3, |
| | | MCM5, MFN2, ATAD3B, |
| | | DDX3X, PSMC4, ARF3, DHX15, |
| triphosphate hydrolase | | CHD3, DNM2 |
| GO: 0000082~G1/S transition | 5 | PRIM1, CUL4A, CRLF3, MCM4, |
| of mitotic cell cycle | | MCM5 |
| IPR024156: Small GTPase | 3 | ARF3, ARL16, ARF5 |
| superfamily, ARF type | | |
| GO: 0005762~mitochondrial | 3 | MRPL22, MRPL13, MRPL28 |
| large ribosomal subunit | | |
| Apoptosis | 13 | BCL10, FOXO1, PTRH2, |
| | | NOC2L, MFN2, EI24, CASP3, |
| | | CSNK2A1, DDX3X, PPID, |
| | | TRAF4, C1D, THOC1 |
| GO: 0043488~regulation of | 5 | PSMB4, PSMD14, PSMB7, |
| mRNA stability | | PSMC4, APEX1 |
| IPR004217: Tim10/DDP | 2 | TIMM9, TIMM10 |
| family zinc finger | | |
| IPR003152: PIK-related | 2 | SMG1, PRKDC |
| kinase, FATC | | |
| IPR014009: PIK-related | 2 | SMG1, PRKDC |
| kinase | | |
| IPR018525: Mini- | 2 | MCM4, MCM5 |
| chromosome maintenance, | | |
| conserved site | | |
| hsa05016: Huntington's | 8 | NDUFS6, NDUFA2, CASP3, |
| disease | | NDUFB6, POLR2K, RCOR1, |
| | | ATP5C1, SOD2 |
| lipid moiety-binding region: | 5 | HPCAL1, GNAI2, ARF3, |
| N-myristoyl glycine | | PPP3R1, ARF5 |
| GO: 0004003~ATP- | 3 | DDX3X, MCM4, CHD3 |
| dependent DNA helicase | | |
| activity | | |
| h_fasPathway: FAS signaling | 3 | CASP3, LMNA, PRKDC |
| pathway ( CD95 ) | | |
| GO: 0051573~negative | 2 | KDM1A, DNMT1 |
| regulation of histone H3-K9 | | |
| methylation | | |
| GO: 0000395~mRNA | 2 | SRSF1, PRPF39 |
| 5′-splice site recognition | | |
| GO: 0019076~viral release | 2 | IST1, PPID |
| from host cell | | |
| GO: 0046939~nucleotide | 2 | DTYMK, AK2 |
| phosphorylation | | |
| GO: 0061133~endopeptidase | 2 | ADRM1, PSMD14 |
| activator activity | | |
| GO: 0007032~endosome | 3 | STX6, USP8, SNX10 |
| organization | | |
| GO: 0070911~global | 3 | UBE2N, CUL4A, UBE2V2 |
| genome nucleotide-excision | | |
| repair | | |
| IPR006689: Small GTPase | 3 | ARF3, ARL16, ARF5 |
| superfamily, ARF/SAR type | | |
| Magnesium | 13 | GNAI2, FDPS, ATP11B, TKT, |
| | | ACLY, HPRT1, CNOT7, LAP3, |
| | | PPM1K, RFK, ENTPD4, APEX1, |
| | | STK38L |
| repeat: HAT 7 | 2 | PRPF39, PRPF6 |
| region of interest: Flexible | 2 | SMC2, SMC3 |
| hinge | | |
| PIRSF005719: structural | 2 | SMC2, SMC3 |
| maintenance of chromosomes | | |
| protein | | |
| GO: 0042147~retrograde | 4 | STX6, VPS29, STX16, VPS26B |
| transport, endosome to Golgi | | |

TABLE 3
HIV-low cutoff

| Category | Count | Genes |
|---|---|---|
| Enrichment Score: | | |
| 8.518413481739762 | | |
| Zinc-finger | 282 | UTRN, RP9, RNF216, RORA, ZNF638, ZNRF1, BRPF1, CUL9, ZFP90, |
| | | ZNF106, ZNF394, ZNF101, ZNF43, ESCO1, POLK, RNF220, ZC3HC1, PAN3, |
| | | ZNF44, ROCK1, ZNF644, RXRB, ROCK2, ZHX1, VPS41, UBR2, OPTN, |
| | | BRAP, ZNF37A, UHRF2, UBR7, PIAS4, PARP12, MTF2, MLLT10, TRMT13, |
| | | ACAP1, ACAP2, PRDM2, AMFR, PIAS1, ZNHIT3, ZNF131, ZNF511, ASAP1, |
| | | MYO9B, RFFL, DIDO1, NR1H2, TCF20, ARIH2, RCHY1, PLAGL2, ASXL2, |
| | | IKZF5, DNMT3A, ESRRA, ZNF529, IKZF2, ZC3H18, KLF13, ZNF121, KLF10, |
| | | CREBBP, RYBP, ZBTB40, CBLB, RNF4, IRF2BPL, JAZF1, HGS, KAT6B, |
| | | RERE, TAF1B, ZNF292, ZNF534, ZMAT5, ZNF675, HELZ, ZEB1, ZBTB38, |
| | | SMAP1, MBTD1, ASH2L, ZNF148, NSMCE1, NSMCE2, USP16, ZNF493, |
| | | ZFP36, ZCCHC10, BRF1, ZC3H7A, BRF2, POGZ, ZC3H7B, APTX, GTF2H3, |
| | | HERC2, MBD1, GTF2B, PJA2, ASH1L, ZNF277, USP22, ZNF746, ZNF740, |
| | | REV3L, ZNF276, ZNF275, ZNF274, USP3, ZBTB10, ZBTB11, WRNIP1, USP5, |
| | | PML, TRIM14, EEA1, CBLL1, ZNF780B, ZNF780A, POLR2B, MYCBP2, |
| | | ZFP36L2, DGKE, MORC3, GATAD2A, ZSCAN25, THAP1, THAP2, MLLT6, |
| | | BAZ2B, ZNF268, BAZ2A, RASA2, RBM22, BRD1, ZNF28, TRIM27, TRIM26, |
| | | PPP1R10, PHF10, DGKH, TRIM25, SF3A2, ATMIN, TRIM22, ZNF664, |
| | | ZNF672, PLEKHF2, PHF14, YAF2, ARAF, WHSC1L1, MEX3C, DGKZ, |
| | | ZBTB2, ZNF764, ZNF766, BARD1, MKRN1, ZNF583, ZC3HAV1, GATA3, |
| | | RBCK1, RNF149, RBM10, RNF146, PHRF1, MTA2, NEIL2, ZNF814, ZNF7, |
| | | TRERF1, TRIM38, EP300, TRIM33, KDM2A, MIB2, NBR1, RNF139, RNF138, |
| | | SLU7, PYGO2, AKAP8, ZNF587, FBXO11, ZNF586, ZFAND6, TRAF2, |
| | | ARFGAP2, ZFAND5, ZNF430, AGFG2, ZFAND1, ZNF330, CXXC1, RNF166, |
| | | TRIM69, RNF168, RNF10, RNF167, RUNX1, TRAF5, TRAF3, ZMYM2, |
| | | TRIP4, VAV3, ZMYM4, ZMYM5, NR4A1, KAT5, FOXP3, VAV1, MSL2, |
| | | PHF3, PDZD8, PHF1, HIVEP2, HIVEP1, CTCF, ZKSCAN1, CBFA2T2, ZZEF1, |
| | | TRIM4, PCGF5, PEX2, RSPRY1, ZNF721, RANBP2, ZCCHC6, KDM5B, |
| | | KDM5C, ZCCHC7, NFX1, INO80B, EGR1, ZCCHC3, ZFX, PRKCI, PRKCH, |
| | | IRF2BP2, PRKCD, PRKCB, XPA, BPTF, ZFAND2A, ZFAND2B, CPSF4, |
| | | JMJD1C, MDM4, PRKD3, DPF2, ING4, ING3, ING2, KMT2A, XIAP, KMT2C, |
| | | ZNF800, EGLN1, RNF125, RPA1, RNF126, MAP3K1, XAF1, ZC3H12D, |
| | | DUS3L, L3MBTL2, CBL, RAF1, TAB3, ZBED2, RNF115, RPAP2, SP1, |
| | | ZBED5, KDM4C, ARAP2, RNF113A, RNF111 |
| Zinc | 339 | UTRN, RP9, RNF216, RORA, ZNF638, ZNRF1, BRPF1, CUL9, ZFP90, ERAP1, |
| | | ZNF106, ZNF394, ZNF101, ZNF43, ESCO1, POLK, RNF220, PAN3, ZC3HC1, |
| | | ZNF44, ROCK1, ZNF644, RXRB, ROCK2, ZHX1, VPS41, UBR2, OPTN, |
| | | BRAP, ZNF37A, UHRF2, UBR7, PIAS4, PARP12, MTF2, MLLT10, TRMT13, |
| | | ACAP1, ACAP2, PRDM2, PIAS1, AMFR, ZNHIT3, MOB4, ZNF131, ZNF511, |
| | | ASAP1, UBA5, MYO9B, DUSP12, RFFL, DIDO1, NR1H2, TCF20, ARIH2, |
| | | RCHY1, PLAGL2, IKZF5, ASXL2, DNMT3A, ESRRA, ZNF529, IKZF2, |
| | | ZC3H18, KLF13, ZNF121, KLF10, CREBBP, RYBP, ZBTB40, SMAD3, |
| | | CSRP1, CBLB, RNF4, LASP1, IRF2BPL, JAZFl, HGS, KAT6B, RERE, TAF1B, |
| | | SPG7, LIMA1, ZNF292, ZNF534, FAM96A, ZMAT5, ZNF675, HELZ, ZEB1, |
| | | ZBTB38, LNPEP, SMAP1, MBTD1, ASH2L, ZNF148, NSMCE1, NSMCE2, |
| | | USP16, ZNF493, ZFP36, ZCCHC10, BRF1, POGZ, BRF2, ZC3H7A, ZC3H7B, |
| | | POLR1A, GTF2H3, APTX, HERC2, GTF2B, MBD1, RAD50, PJA2, ASH1L, |
| | | ZNF277, USP22, ZNF746, ZNF740, REV3L, ZNF276, ZNF275, LIMS1, |
| | | ZNF274, USP3, ZBTB10, YPEL5, ZBTB11, WRNIP1, USP5, YPEL3, USP4, |
| | | MKNK2, PML, TRIM14, EEA1, CBLL1, QTRT1, ZNF780B, ZNF780A, |
| | | POLR2B, MYCBP2, ZFP36L2, LPXN, DGKE, MORC3, PITRM1, GATAD2A, |
| | | PPP3CB, ZSCAN25, THAP1, THAP2, MLLT6, BAZ2B, ZNF268, BAZ2A, |
| | | RASA2, RBM22, BRD1, ZNF28, TRIM27, TRIM26, PHF10, MSRB1, PPP1R10, |
| | | DGKH, TRIM25, SF3A2, ATMIN, TRIM22, MT1X, ZNF664, DNPEP, ZNF672, |
| | | PLEKHF2, PHF14, YAF2, ARAF, WHSC1L1, MEX3C, DGKZ, NLN, ZBTB2, |
| | | ZNF764, ZNF766, BARD1, MKRN1, ZNF583, ZC3HAV1, IDE, APOBEC3G, |
| | | APOBEC3C, APOBEC3D, GATA3, RBCK1, RNF149, RBM10, RNF146, |
| | | PHRF1, PDXK, MTA2, NEIL2, ZNF814, ZNF7, TRERF1, TIMM8A, TRIM38, |
| | | EP300, TRIM33, KDM2A, MIB2, NBR1, RNF139, RNF138, SLU7, PYGO2, |
| | | AKAP8, ZNF587, FBXO11, ZNF586, ZFAND6, TRAF2, ARFGAP2, ZFAND5, |
| | | ZNF430, ELAC2, AGFG2, ZFAND1, ZNF330, CXXCl, RNF166, TRIM69, |
| | | STAMBPL1, SLC30A5, RNF168, RNF10, RNF167, RUNX1, TRAF5, TRAF3, |
| | | SETDB1, ZMYM2, TRIP4, VAV3, EHMT1, RABIF, ZMYM4, ZMYM5, |
| | | NR4A1, KAT5, FOXP3, VAV1, MSL2, PDZD8, PHF3, PHF1, MIR, HIVEP2, |
| | | HIVEP1, UTY, CTCF, ZKSCAN1, CBFA2T2, ZZEF1, TRIM4, PCGF5, PEX2, |
| | | RSPRY1, MOB3A, ZYX, ZNF721, RANBP2, ZCCHC6, KDM5B, KDM5C, |
| | | ZCCHC7, NFX1, INO80B, EGR1, ZCCHC3, ZFX, PRKCI, PRKCH, IRF2BP2, |
| | | PRKCD, PRKCB, HAGH, XPA, BPTF, ZFAND2A, ZFAND2B, MDM4, CPSF4, |
| | | JMJD1C, CPSF3, PRKD3, ABLIM1, DPF2, ING4, ING3, MOB1B, ING2, |
| | | KMT2A, XIAP, KMT2C, MGMT, ZNF800, EGLN1, NGLY1, RNF125, RPA1, |
| | | RNF126, MAP3K1, SLC39A6, CCS, XAF1, SLC39A3, ZC3H12D, DUS3L, |
| | | STAMBP, DCTD, L3MBTL2, CBL, SAMHD1, RAF1, SIRT6, SIRT7, TAB3, |
| | | SIRT2, ZBED2, RNF115, RPAP2, SP1, ZBED5, KDM4C, ARAP2, RNF113A, |
| | | RNF111 |
| GO: 0008270~zinc | 202 | UTRN, ZNF638, RORA, ZNRF1, BRPF1, CUL9, ERAP1, ZC3HC1, RNF220, |
| ion binding | | RXRB, UBR2, VPS41, BRAP, UHRF2, PIAS4, UBR7, MTF2, MLLT10, |
| | | PRDM2, AMFR, PIAS1, DUSP12, OAS1, MYO9B, OAS2, RFFL, DIDO1, |
| | | NR1H2, TCF20, ARIH2, RCHY1, ESRRA, CREBBP, RYBP, SMAD3, CSRP1, |
| | | CBLB, RNF4, LASP1, KAT6B, RERE, LIMA1, SPG7, ZMAT5, ZNF675, ZEB1, |
| | | PTER, LNPEP, MBTD1, NSMCE1, NSMCE2, USP16, ZCCHC10, BRF1, |
| | | ZDHHC3, BRF2, ZDHHC8, POLR1A, HERC2, MBD1, GTF2B, TTF2, PJA2, |
| | | CHMP1A, ZDHHC16, ASH1L, ZDHHC12, USP22, ZNF276, LIMS1, USP3, |
| | | USP5, TRIM14, PML, EEA1, CBLL1, MYCBP2, LPXN, MORC3, PITRM1, |
| | | GATAD2A, THAP1, BAZ2B, MLLT6, BAZ2A, BRD1, TRIM27, TRIM26, |
| | | MSRB1, PHF10, TRIM25, SF3A2, TRIM22, MT1X, DNPEP, PHF14, YAF2, |
| | | WHSC1L1, MEX3C, BARD1, MKRN1, CNDP2, IDE, APOBEC3G, |
| | | APOBEC3C, APOBEC3D, GATA3, RBCK1, RNF149, RBM10, RNF146, |
| | | PHRF1, PDXK, MTA2, NEIL2, TRIM38, EP300, KDM2A, TRIM33, MIB2, |
| | | NBR1, RNF139, PYGO2, SLU7, RNF138, AKAP8, FBXO11, TRAF1, ZFAND6, |
| | | TRAF2, ZFAND5, ZFAND1, ZNF330, CXXCL RNF166, TRIM69, SLC30A5, |
| | | RNF10, RNF168, RNF167, TRAF5, TRAF3, SETDB1, ZMYM2, EHMT1, |
| | | TRIP4, ZMYM4, RABIF, ZMYM5, NR4A1, MSL2, PHF3, PHF1, MTR, |
| | | CRYZL1, UQCRC1, CTCF, ZZEF1, TRIM4, PCGF5, RSPRY1, PEX2, |
| | | RANBP2, ZYX, KDM5B, ZCCHC6, ZCCHC7, KDM5C, NFX1, EGR1, |
| | | ZCCHC3, PRKCB, BPTF, ZFAND2A, ZFAND2B, COMMD3, CPSF4, MDM4, |
| | | DPF2, ABLIM1, ING4, ING3, ING2, XIAP, KMT2A, KMT2C, RNF125, |
| | | RNF126, MAP3K1, CCS, XAF1, DCTD, L3MBTL2, CBL, SAMHD1, SIRT6, |
| | | SIRT2, TAB3, RNF115, KDM4C, RNF113A, RNF111 |
| Metal-binding | 467 | RP9, ZNF638, RORA, OGDH, CIAPIN1, BRPF1, PGP, CUL9, ZNF106, |
| | | ZNF394, MAP2K7, ZNF101, ZNF43, ZNF44, ROCK1, ZN1F644, RXRB, |
| | | ROCK2, ZHX1, VPS41, BRAP, MARK2, ZNF37A, NME3, ZNHIT3, MOB4, |
| | | ZNF131, ZNF511, UBA5, DUSP12, DIDO1, ARIH2, RCHY1, ASXL2, |
| | | DNMT3A, MGAT4A, ESRRA, ZNF529, ZC3H18, KLF13, ZNF121, KLF10, |
| | | ATP11A, HSPB11, LASP1, HGS, RERE, TAF1B, LIMA1, ZNF292, ZNF534, |
| | | ZNF675, LATS1, LNPEP, SMAP1, ZNF148, USP16, ZNF493, BRF1, ZC3H7A, |
| | | BRF2, ZC3H7B, GTF2H3, PAPD5, PJA2, ZNF277, CHSY1, ZNF746, USP22, |
| | | ZNF740, ZNF276, ZNF275, ZNF274, REPS1, WRNIP1, MKNK2, PML, |
| | | TRIM14, EEA1, ERI3, CBLL1, QTRT1, ZNF780B, HSCB, ZNF780A, |
| | | MYCBP2, SNRK, DGKE, PITRM1, PPP2CB, PPP3CB, ILVBL, ZSCAN25, |
| | | ZNF268, C1GALT1, RASA2, BRD1, MAT2A, ZNF28, TRIM27, TRIM26, |
| | | PPP1R10, PHF10, DGKH, TRIM25, SF3A2, PCK2, TRIM22, MT1X, ZNF664, |
| | | RPS6KA3, ZNF672, PLEKHF2, PHF14, SDHC, ARAF, MCFD2, WHSC1L1, |
| | | DGKZ, ZBTB2, ZNF764, ZNF766, BARD1, GNA13, ZNF583, CNOT8, |
| | | NT5C3A, ATOX1, CNDP2, IDE, IDH3G, PDE4B, CTDSP1, ENOPH1, RNF149, |
| | | SAR1B, RNF146, PHRF1, NUDT1, PDXK, NUDT4, ACTN4, MTA2, NUDT5, |
| | | NEIL2, STIM1, PDE4D, ZNF7, TATDN3, EP300, KDM2A, MIB2, RNF139, |
| | | RNF138, ZNF587, FBXO11, ZNF586, ARFGAP2, TRAF2, ZNF430, ELAC2, |
| | | ME2, ZNF330, CXXC1, RNF166, RNF168, LIAS, RNF167, TRAF5, SDF4, |
| | | TRAF3, B4GALT3, VAV3, TRIP4, EHMT1, SYT11, IREB2, NR4A1, HDDC3, |
| | | VAV1, FURIN, PDZD8, PDE7A, NDUFV2, HIVEP2, HIVEP1, ABL2, DICER1, |
| | | CTCF, ZKSCAN1, TRIM4, PEX2, CDK5RAP1, KDM5B, ZCCHC6, NDUFS1, |
| | | KDM5C, ZCCHC7, NFX1, INO80B, ZCCHC3, ZFX, IRF2BP2, CDK2, ARL3, |
| | | HAGH, ZFAND2A, ZFAND2B, CPSF4, JMJD1C, CPSF3, ABLIM1, DPF2, |
| | | GALNT2, KMT2A, ETHE1, KMT2C, PPM1A, EGLN1, NGLY1, RNF125, |
| | | RNF126, CCS, XAF1, EHD1, EHD4, ALKBH7, CBL, ANXA1, SAMHD1, |
| | | RAF1, TAB3, ADI1, ZBED2, RNF115, ZBED5, KDM4C, JAK2, ALKBH5, |
| | | RNF113A, RNF111, RCN2, S100A4, ADCY7, UTRN, RNASEH1, RNF216, |
| | | ZNRF1, ATP2B4, ZFP90, ERAP1, YDJC, ESCO1, POLK, RNF220, ZC3HC1, |
| | | PAN3, CAPNS1, TRABD2A, PIM1, POLB, UBR2, OPTN, MGAT1, UHRF2, |
| | | UBR7, PIAS4, PARP12, ATP2C1, MTF2, TRMT13, ACAP1, MLLT10, PGM1, |
| | | ACAP2, FBXL5, PRDM2, AMFR, PIAS1, ASAP1, OAS1, MYO9B, RFFL, |
| | | OAS2, PPAT, NR1H2, TCF20, KRAS, IDH2, PLAGL2, IKZF5, IKZF2, |
| | | CREBBP, S100A11, RYBP, ZBTB40, SMAD3, OXSR1, CSRP1, CBLB, |
| | | ATP13A1, RNF4, IRF2BPL, DCP2, JAZFl, KAT6B, SPG7, FAM96A, ZMAT5, |
| | | HELZ, ZEB1, ZBTB38, EFHD2, PTER, MBTD1, ASH2L, NSMCE1, NSMCE2, |
| | | TOP2B, NT5C, ZFP36, ZCCHC10, POGZ, POLR1A, APTX, CYB5A, HERC2, |
| | | MBD1, GTF2B, RAD50, ASH1L, MAP3K13, REV3L, LIMS1, USP3, ZBTB10, |
| | | YPEL5, ZBTB11, USP5, USP4, YPEL3, CETN2, POLR2B, ZFP36L2, LPXN, |
| | | GNPTAB, MORC3, TYW1, GATAD2A, AGO2, THAP1, PRKAA1, THAP2, |
| | | NENF, MLLT6, BAZ2B, BAZ2A, RBM22, MSRB1, ATMIN, DNPEP, JMJD6, |
| | | YAF2, MEX3C, NLN, MKRN1, ZC3HAV1, APOBEC3G, APOBEC3C, |
| | | APOBEC3D, GATA3, RBCK1, RBM10, ZNF814, CHP1, PPP1CB, TRERF1, |
| | | TIMM8A, TRIM38, TRIM33, NBR1, SLU7, PYGO2, AKAP8, PRPS2, PRPS1, |
| | | ZFAND6, ZFAND5, AGFG2, ITGAE, AGMAT, ZFAND1, ITGB1, PEF1, |
| | | TRIM69, CNOT6L, STAMBPL1, RNF10, RUNX1, SETDB1, ZMYM2, RABIF, |
| | | ZMYM4, ZMYM5, KAT5, FOXP3, MSL2, PHF3, PHF1, MTR, RHOT1, |
| | | RHOT2, UTY, FOXK2, UQCRFS1, ZZEF1, CBFA2T2, GSS, PCGF5, TPP1, |
| | | RSPRY1, MOB3A, DBR1, ATP8B2, ZYX, ZNF721, RANBP2, EGR1, PFKL, |
| | | PRKCI, PRKCH, PRKCD, PRKCB, NUCB1, XPA, BPTF, NUCB2, COMMD1, |
| | | MDM4, PRKD3, ING4, GLRX5, MOB1B, ING3, ING2, XIAP, MGMT, |
| | | ZNF800, RSAD2, GLRX2, RPA1, MTHFS, MAP3K3, MAP3K1, ZC3H12D, |
| | | SCO2, DUS3L, STAMBP, DCTD, L3MBTL2, SIRT6, SIRT7, SIRT2, RPAP2, |
| | | SP1, TDP2, ARAP2 |
| Enrichment Score: | | |
| 6.51824669041824 | | |
| IPR019787: Zinc | 31 | DPF2, ING4, ING3, ING2, KMT2A, KMT2C, DIDO1, CXXC1, BRPF1, BAZ2B, |
| finger, PHD-finger | | MLLT6, KDM5B, BAZ2A, KDM5C, NFX1, BRD1, PHRF1, PHF10, PHF3, |
| | | UHRF2, KDM2A, PHF14, BPTF, TRIM33, PHF1, MTF2, MLLT10, WHSC1L1, |
| | | ASH1L, PYGO2, KAT6B |
| SM00249: PHD | 32 | DPF2, ING4, ING3, ING2, KMT2A, KMT2C, DIDO1,CXXC1, TCF20, BRPF1, |
| | | BAZ2B, MLLT6, KDM5B, BAZ2A, KDM5C, BRD1, PHRF1, PHF10, PHF3, |
| | | UHRF2, KDM2A, BPTF, TRIM33, PHF14, PHF1, MTF2, MLLT10, WHSC1L1, |
| | | ASH1L, KDM4C, PYGO2, KAT6B |
| IPR001965: Zinc | 32 | DPF2, ING4, ING3, ING2, KMT2A, KMT2C, DIDO1, CXXC1, TCF20, BRPF1, |
| finger, PHD-type | | BAZ2B, MLLT6, KDM5B, BAZ2A, KDM5C, BRD1, PHRF1, PHF10, PHF3, |
| | | UHRF2, KDM2A, BPTF, TRIM33, PHF14, PHF1, MTF2, MLLT10, WHSC1L1, |
| | | ASH1L, KDM4C, PYGO2, KAT6B |
| IPR011011: Zinc | 38 | DPF2, ING4, ING3, ING2, KMT2A, KMT2C, EEA1, RFFL, DIDO1, CXXC1, |
| finger, FYVE/PHD- | | BRPF1, BAZ2B, MLLT6, KDM5B, BAZ2A, KDM5C, BRD1, PHRF1, CREBBP, |
| type | | PHF10, PHF3, PLEKHF2, UHRF2, KDM2A, UBR7, BPTF, TRIM33, PHF14, |
| | | PHF1, MTF2, MLLT10, WHSC1L1, ASH1L, KDM4C, HGS, PYGO2, SYTL3, |
| | | KAT6B |
| IPR019786: Zinc | 23 | ING4, BRD1, PHRF1, ING3, ING2, DIDO1, CXXC1, BRPF1, PHF3, PHF14, |
| finger, PHD-type, | | KDM2A, BPTF, PHF1, TRIM33, MTF2, MLLT10, WHSC1L1, ASH1L, PYGO2, |
| conserved site | | MLLT6, KDM5B, KDM5C, NFX1 |
| zinc finger | 14 | DPF2, PHF14, BPTF, KMT2A, PHF1, MTF2, MLLT10, KMT2C, WHSC1L1, |
| region: PHD-type 2 | | KDM4C, KAT6B, MLLT6, KDM5B, KDM5C |
| zinc finger | 14 | DPF2, PHF14, BPTF, KMT2A, PHF1, MTF2, MLLT10, KMT2C, WHSC1L1, |
| region: PHD-type 1 | | KDM4C, KAT6B, MLLT6, KDM5B, KDM5C |
| zinc finger | 16 | BRD1, ING4, PHRF1, ING3, ING2, DIDO1, CXXC1, BRPF1, PHF3, UHRF2, |
| region: PHD-type | | KDM2A, TRIM33, ASH1L, PYGO2, BAZ2B, BAZ2A |
| Enrichment Score: | | |
| 6.341978088960009 | | |
| Ubl conjugation | 142 | MKRN1, RNF216, SAE1, ZNRF1, CUL3, CUL2, CUL9, KLHL9, FBXO25, |
| pathway | | RBCK1, RNF149, RNF146, ZC3HC1, RNF220, SOCS3, ANAPC4, SOCS1, |
| | | UBE2J1, UBR2, UBE2J2, BRAP, TRIM38, UHRF2, KDM2A, UBR7, PIAS4, |
| | | TRIM33, MIB2, FBXL5, RNF139, RNF138, PIAS1, AMFR, FBXO11, TRAF2, |
| | | ZFAND5, UBA5, ANAPC10, KEAP1, RFFL, COMMD9, UBAC1, COMMD10, |
| | | UBE2D4, FBXW7, ARIH2, KRAS, FBXW5, TRIM69, FBXO6, STAMBPL1, |
| | | FBXW2, HECTD4, RNF168, RCHY1, RNF167, TRAF3, HECTD1, SPOP, |
| | | PELI1, KIAA1586, CDC23, MALT1, CDC27, ATE1, MSL2, CBLB, RNF4, |
| | | UBA3, SMURF2, FBXO34, UBE2E1, UBE2G1, BAP1, TRIM4, NSMCE2, |
| | | RANBP2, USP16, FBXL15, USP15, DCAF16, DCAF15, NFX1, VCPIP1, |
| | | TBL1XR1, UBE2A, HERC6, HERC5, HERC2, PJA2, WDR48, UFL1, ATG4B, |
| | | MED8, DDB2, COMMD3, UBE2W, COMMD1, UCHL3, CANDI, USP22, |
| | | USP24, OTUD5, UBE2Z, USP3, XIAP, USP5, USP4, CBLL1, FEM1B, STUB1, |
| | | FEM1A, UBE2R2, MYCBP2, PRPF19, RNF125, RNF126, USP36, USP34, |
| | | FBXW11, STAMBP, WDTC1, USP40, UBE4A, VHL, LRRC41, CBL, TRIM27, |
| | | BIRC6, TRIM25, TRIM22, NAE1, WSB1, RNF115, USP47, MEX3C, |
| | | TRPC4AP, CUL4B, TBL1X, USP42, RNF111, BARD1 |
| IPR013083: Zinc | 96 | MKRN1, ZNRF1, TRIM4, PCGF5, BRPF1, PEX2, RSPRY1, NSMCE2, RBCK1, |
| finger, | | RNF149, USP16, KDM5B, KDM5C, RNF146, PHRF1, RNF220, VPS41, BRAP, |
| RING/FYVE/PHD- | | PJA2, TRIM38, UHRF2, KDM2A, PIAS4, TRIM33, BPTF, UBR7, MTF2, MIB2, |
| type | | MLLT10, ASH1L, COMMD3, RNF139, PYGO2, RNF138, MDM4, USP22, |
| | | AMFR, ZNHIT3, DPF2, TRAF2, ING4, ING3, ING2, KMT2A, USP3, USP5, |
| | | KMT2C, PML, EEA1, RFFL, CBLL1, STUB1, DIDO1, CXXC1, MYCBP2, |
| | | RNF125, PRPF19, RNF126, ARIH2, RNF166, TRIM69, MAP3K1, RNF10, |
| | | RNF168, RNF167, RCHY1, MLLT6, BAZ2B, TRAF5, BAZ2A, TRAF3, BRD1, |
| | | UBE4A, CBL, CREBBP, TRIM27, TRIM26, PHF10, TRIM25, TRIM22, CBLB, |
| | | PHF3, RNF115, PLEKHF2, PHF1, PHF14, RNF4, WHSC1L1, MEX3C, HGS, |
| | | KDM4C, SYTL3, KAT6B, RNF113A, BARD1, RNF111 |
| Ligase | 76 | MKRN1, SAE1, RNF216, ZNRF1, GSS, TRIM4, NSMCE1, RBCK1, NSMCE2, |
| | | RNF149, RANBP2, RNF146, NFX1, RNF220, HERC6, HERC5, UBR2, HERC2, |
| | | GMPS, BRAP, UFL1, PJA2, TRIM38, GLUL, UHRF2, PIAS4, TRIM33, UBR7, |
| | | MIB2, FARSB, RNF139, RNF138, AMFR, YARS2, PIAS1, PCCB, TRAF2, |
| | | XIAP, FARS2, WARS2, RFFL, CBLL1, STUB1, MYCBP2, RNF125, PRPF19, |
| | | MTHFS, RNF126, ARIH2, TRIM69, HECTD4, RNF168, RNF167, RCHY1, |
| | | ACSL4, ACSL3, ACSL5, TRAF3, HECTD1, PELI1, KIAA1586, UBE4A, CBL, |
| | | TRIM27, BIRC6, TRIM25, TRIM22, MSL2, CBLB, RNF115, RNF4, UBA3, |
| | | MEX3C, SMURF2, BARD1,RNF111 |
| GO: 0016874~ligase | 63 | MKRN1, RNF216, ZNRF1, TRIM4, NSMCE1, RBCK1, NSMCE2, RNF149, |
| activity | | RANBP2, RNF146, NFX1, RNF220, HERC6, HERC5, UBR2, HERC2, BRAP, |
| | | PJA2, UFL1, TRIM38, UHRF2, PIAS4, TRIM33, UBR7, MIB2, RNF139, |
| | | RNF138, AMFR, PIAS1, PCCB, TRAF2, XIAP, RFFL, CBLL1, STUB1, |
| | | MYCBP2, PRPF19, RNF125, RNF126, ARIH2, TRIM69, HECTD4, RNF168, |
| | | RNF167, RCHY1, ACSL3, TRAF3, HECTD1, PELI1, KIAA1586, UBE4A, CBL, |
| | | TRIM27, BIRC6, TRIM22, MSL2, CBLB, RNF115, RNF4, MEX3C, SMURF2, |
| | | BARD1, RNF111 |
| GO: 0004842~ubiquitin- | 71 | MKRN1, BACH2, UBE2G1, RNF216, ZNRF1, CUL3, NSMCE1, KLHL9, |
| protein | | FBXO25, RBCK1, KLHL24, FBXL15, RNF146, UBE2A, RNF220, HERC6, |
| transferase activity | | ANAPC4, HERC5, UBR2, HERC2, BRAP, PJA2, UHRF2, TRIM33, MIB2, |
| | | DDB2, RNF139, FBXL5, UBE2W, AMFR, FBXO11, TRAF2, XIAP, KEAP1, |
| | | CBLL1, FEM1B, STUB1, FEM1A, UBE2R2, PRPF19, UBE2D4, ARIH2, |
| | | FBXW7, KBTBD2, TRIM69, FBXO6, FBXW2, HECTD4, RNF10, RNF168, |
| | | RNF167, RCHY1, TRAF5, FBXW11, TRAF3, HECTD1, VHL, CBL, TRIM27, |
| | | CDC23, BIRC6, MALT1, TRIM25, TSPAN17, WSB1, CBLB, RNF115, RNF4, |
| | | SMURF2, UBE2E1, BARD1 |
| GO: 0016567~protein | 73 | BACH2, SAE1, CUL3, TRIM4, NSMCE1, CUL9, KLHL9, FBXO25, KLHL24, |
| ubiquitination | | RNF149, FBXL15, DCAF16, VCPIP1, DCAF15, ZC3HC1, RNF220, SOC53, |
| | | SOCS1, UBE2J1, MED11, HERC2, TMEM189, BRAP, PJA2, UHRF2, TRIM33, |
| | | UBR7, MED17, MED8, FBXL5, RNF139, RNF138, CAND1 NFE2L2, MDM4, |
| | | MED1, FBXO11, XIAP, KEAP1, CBLL1, UBAC1, FEM1B, STUB1, FEM1A, |
| | | MYCBP2, UBE2D4, ARIH2, FBXW7, KBTBD2, FBXW5, FBXW2, RNF168, |
| | | RCHY1, TRAF5, FBXW11, TRAF3, WDTC1, VHL, LRRC41, SPSB3, CBL, |
| | | BIRC6, MALT1, TSPAN17, TRIM22, WSB1, MED31, MSL2, RNF4, |
| | | TRPC4AP, UBE2E1, BARD1, RNF111 |
| IPR001841: Zinc | 58 | MKRN1, ZNRF1, TRIM4, PCGF5, RSPRY1, PEX2, CUL9, NSMCE1, RBCK1, |
| finger, RING-type | | RNF149, RNF146, NFX1, PHRF1, RNF220, VPS41, BRAP, PJA2, TRIM38, |
| | | UHRF2, TRIM33, MIB2, COMMD3, RNF139, RNF138, AMFR, MDM4, |
| | | TRAF2, XIAP, KMT2C, PML, RFFL, CBLL1, MYCBP2, RNF125, RNF126, |
| | | ARIH2, RNF166, TRIM69, MAP3K1, RNF168, RNF10, RCHY1, RNF167, |
| | | TRAF5, TRAF3, CBL, TRIM27, TRIM26, TRIM25, TRIM22, MSL2, CBLB, |
| | | RNF115, RNF4, MEX3C, RNF113A, RNF111, BARD1 |
| SM00184: RING | 48 | MKRN1, TRAF2, XIAP, KMT2C, PML, RFFL, ZNRF1, MYCBP2, TRIM4, |
| | | RNF125, PCGF5, RNF126, ARIH2, RNF166, RSPRY1, PEX2, TRIM69, |
| | | RBCK1, RNF168, RNF10, RCHY1, RNF149, RNF167, TRAF5, RNF146, NFX1, |
| | | PHRF1, CBL, TRIM27, TRIM26, TRIM25, TRIM22, BRAP, PJA2, TRIM38, |
| | | CBLB, UHRF2, RNF115, TRIM33, RNF4, MIB2, COMMD3, MEX3C, RNF139, |
| | | RNF138, AMFR, RNF113A, RNF111 |
| zinc finger | 44 | MKRN1, TRAF2, CHMP3, XIAP, KMT2C, PML, RFFL, CBLL1, TRIM4, |
| region: RING-type | | RNF125, PCGF5, RNF126, RNF166, RSPRY1, PEX2, TRIM69, MAP3K1, |
| | | RBCK1, RNF168, RNF10, RCHY1, TRAF5, RNF146, TRAF3, RNF220, CBL, |
| | | TRIM27, TRIM26, TRIM25, TRIM22, BRAP, TRIM38, MSL2, CBLB, UHRF2, |
| | | RNF115, TRIM33, RNF4, MEX3C, RNF138, MDM4, AMFR, RNF113A, |
| | | BARD1 |
| IPR017907: Zinc | 30 | MKRN1, TRAF2, PML, CBLL1, RNF125, TRIM4, PCGF5, ARIH2, RNF166, |
| finger, RING-type, | | PEX2, TRIM69, CUL9, RBCK1, RNF10, TRAF5, RNF146, TRAF3, PHRF1, |
| conserved site | | CBL, TRIM27, TRIM25, TRIM22, TRIM38, CBLB, UHRF2, TRIM33, RNF4, |
| | | COMMD3, BARD1, RNF113A |
| Enrichment Score: | | |
| 5.35889838477318 | | |
| GO: 0051607~defense | 43 | ABCF3, CD8A, IFITM1, ZC3HAV1, IFITM2, UNC93B1, PML, BNIP3, RSAD2, |
| response to virus | | OAS1, APOBEC3G, OAS2, APOBEC3C, APOBEC3D, SERINC3, NLRC5, |
| | | BCL2, C19ORF66, IFNG, PYCARD, MX1, MX2, POLR3F, POLR3H, RELA, |
| | | FAM111A, EXOSC5, HERC5, SAMHD1, FADD, TRIM25, POLR3C, TRIM22, |
| | | POLR3E, IFNAR1, IFIT3, IFNAR2, PLSCR1, UNC13D, IFIT5, BNIP3L, IRF3, |
| | | GBP3 |
| Antiviral defense | 31 | ABCF3, IFITM1, ZC3HAV1, IFITM2, UNC93B1, PML, RSAD2, OAS1, |
| | | APOBEC3G, OAS2, APOBEC3C, APOBEC3D, SERINC3, C19ORF66, IFNG, |
| | | MX1, MX2, POLR3F, POLR3H, FAM111A, HERC5, SAMHD1, TRIM25, |
| | | POLR3C, TRIM22, POLR3E, IFIT3, PLSCR1, IFIT5, IRF3, GBP3 |
| Innate immunity | 50 | ZC3HAV1, APOBEC3G, APOBEC3C, APOBEC3D, TRIM4, NLRC5, |
| | | ANKRD17, GATA3, MX1, MX2, IRAK1, LY96, HERC5, FADD, ECSIT, CD84, |
| | | TRIM38, CHID1, RIPK2, AKAP8, IFITM1, IFITM2, CSF1, PML, UNC93B1, |
| | | RSAD2, OAS1, OA5S, SEC14L1, SERINC3, IRAK4, PSTPIP1, PYCARD, MR1, |
| | | TBKBP1, POLR3F, POLR3H, ANXA1, MSRB1, SAMHD1, TRIM25, SLAMF7, |
| | | POLR3C, POLR3E, SIRT2, IFIT3, CD55, IFIT5, JAK2, IRF3 |
| Enrichment Score: | | |
| 5.143098283847529 | | |
| Cell cycle | 120 | ITGB3BP, CHMP3, MAU2, KNTC1, INO80, CASP8AP2, KLHL9, RALB, |
| | | VPS4A, TLK1, CDCA4, STAG1, ESCO1, ZC3HC1, ANAPC4, RINT1, PIM1, |
| | | HMG20B, PPP1CB, MAPK1, UHRF2, EP300, RCC2, MAPK6, PRCC, BIN3, |
| | | CDCA7L, PDCD6IP, ARL8B, MPLKIP, CACUL1, STK10, AHCTF1, ARF6, |
| | | CEP164, ANAPC10, CCNG1, CCNG2, NIPBL, PPP2R2D, SSSCA1, CINP, |
| | | WDR6, CDC23, PMF1, CDC27, ATM, CDKN1B, DMTF1, UBA3, CCNT2, |
| | | E2F3, E2F4, TSG101, CCNT1, LATS1, NDE1, NSMCE2, CDK10, USP16, |
| | | CDK13, ARL2, RBBP4, POGZ, CCNH, DYNLT3, PKN2, DYNLT1, PAPD5, |
| | | BANP, CDK7, PRKCD, RAD50, CDK2, MCM6, ARL3, GAK, SASS6, |
| | | CHMP1A, NSL1, CDK11B, MAPRE2, WASL, USP22, MAPRE1, PDCD2L, |
| | | SPAST, HAUS3, ING4, HAUS6, RABGAP1, ASUN, USP3, HAUS2, HAUS1, |
| | | CETN2, NUMA1, MAP10, TSPYL2, MDC1, RB1CC1, NPAT, PAFAH1B1, |
| | | THAP1, FBXW11, TERF2, TERF1, CSNK1A1, BOD1, PDS5B, SMC5, BIRC6, |
| | | RGS14, SIRT2, NAE1, SMC4, RPS6KA3, MAPK13, CUL4B, C9ORF69 |
| Cell division | 73 | ITGB3BP, CCNT2, CHMP3, MAU2, TSG101, CCNT1, KNTC1, INO80, LATS1, |
| | | NDE1, KLHL9, RALB, VPS4A, NSMCE2, CDK10, USP16, CDCA4, CDK13, |
| | | STAG1, ZC3HC1, POGZ, ANAPC4, PKN2, DYNLT3, DYNLT1, PAPD5, |
| | | CDK7, PPP1CB, CDK2, ARL3, CHMP1A, RCC2, NSL1, BIN3, CDK11B, |
| | | MAPRE2, CDCA7L, ARL8B, WASL, MAPRE1, PDCD6IP, SPAST, HAUS3, |
| | | HAUS6, ASUN, MPLKIP, HAUS2, HAUS1, CETN2, AHCTF1, ARF6, |
| | | ANAPC10, CEP164, CCNG1, CCNG2, NUMA1, MAP10, PAFAH1B1, |
| | | PPP2R2D, TERF1, CSNK1A1, SSSCA1, BOD1, PDS5B, CINP, SMC5, CDC23, |
| | | BIRC6, PMF1, CDC27, SIRT2, RGS14, SMC4 |
| GO: 0051301~cell | 66 | ITGB3BP, CCNT2, MAU2, TSG101, CCNT1, KNTC1, INO80, LATS1, NDE1, |
| division | | NSMCE2, VPS4A, CDK10, TUBA1A, USP16, CDCA4, TUBA1C, CDK13, |
| | | STAG1, ZC3HC1, POGZ, ANAPC4, PKN2, DYNLT3, PAPD5, DYNLT1, |
| | | CDK7, PPP1CB, CDK2, CHMP1A, RCC2, NSL1, CDK11B, MAPRE2, |
| | | CDCA7L, ARL8B, WASL, MAPRE1, HAUS3, HAUS6, ASUN, MPLKIP, |
| | | HAUS2, HAUS1, CETN2, ARF6, ANAPC10, CEP164, CCNG1, CCNG2, |
| | | NUMA1, MAP10, PPP2R2D, TERF1, CSNK1A1, SSSCA1, BOD1, PDS5B, |
| | | CINP, SMC5, CDC23, BIRC6, PMF1, CDC27, SIRT2, RGS14, SMC4 |
| GO: 0007067~mitotic | 49 | ITGB3BP, HAUS3, HAUS6, ASUN, MPLKIP, HAUS2, HAUS1, KNTC1, |
| nuclear division | | CETN2, ANAPC10, CEP164, CLTC, CCNG1, CCNG2, LATS1, OFD1, |
| | | NUMA1, FBXW5, KLHL9, NSMCE2, PAFAH1B1, USP16, PPP2R2D, STAG1, |
| | | TERF1, VCPIP1, CSNK1A1, SSSCA1, BOD1, ZC3HC1, TADA2A, ANAPC4, |
| | | SMC5, DYNLT3, CDC23, BIRC6, PAPD5, DYNLT1, PMF1, SIRT2, RGS14, |
| | | CDK2, RCC2, NSL1, CDK11B, MAPRE2, MAPRE1, WASL, AKAP8 |
| Mitosis | 46 | ITGB3BP, HAUS3, HAUS6, ASUN, MPLKIP, MAU2, HAUS2, HAUS1, |
| | | KNTC1, INO80, CETN2, CEP164, ANAPC10, CCNG1, CCNG2, LATS1, |
| | | NUMA1, NDE1, KLHL9, NSMCE2, PAFAH1B1, USP16, PPP2R2D, STAG1, |
| | | TERF1, CSNK1A1, SSSCA1, BOD1, ZC3HC1, PDS5B, ANAPC4, SMC5, |
| | | DYNLT3, CDC23, BIRC6, PAPD5, DYNLT1, PMF1, SIRT2, CDK2, SMC4, |
| | | RCC2, NSL1, MAPRE2, MAPRE1, WASL |
| Enrichment Score: | | |
| 5.023865225499397 | | |
| DNA damage | 68 | RAD51C, INO80, NSMCE1, AEN, NSMCE2, TLK1, BRD4, INO80D, INO80C, |
| | | INO80B, POLK, UBE2A, NEIL2, FMR1, APTX, GTF2H3, CDK9, MBD4, |
| | | POLB, HERC2, CDK7, CDK2, RAD50, RAD1, XPA, NABP1, XPC, HIPK2, |
| | | DDB2, RNF138, UBE2W, PSME4, REV3L, USP3, WRNIP1, HUS1, MGMT, |
| | | CETN2, MUM1, CEP164, MAPKAPK2, STUB1, XAB2, PRPF19, RPA1, |
| | | CHD1L, MDC1, FBXO6, RNF168, ACTL6A, ERCC3, MSH6, MSH2, TAOK1, |
| | | TP53BP1, CINP, SMC5, SMC6, ATMIN, UIMC1, ATM, MPG, PHF1, TDP2, |
| | | U5P47, CUL4B, OGG1, BARD1 |
| DNA repair | 56 | RAD51C, INO80, NSMCE1, NSMCE2, INO80D, INO80C, INO80B, POLK, |
| | | UBE2A, NEIL2, APTX, GTF2H3, CDK9, MBD4, POLB, HERC2, CDK7, |
| | | CDK2, RAD50, RAD1, XPA, NABP1, XPC, DDB2, UBE2W, RNF138, PSME4, |
| | | REV3L, MGMT, CETN2, MUM1, CEP164, STUB1, XAB2, RPA1, PRPF19, |
| | | CHD1L, MDC1, FBXO6, RNF168, ACTL6A, ERCC3, MSH6, TAOK1, MSH2, |
| | | TP53BP1, CINP, SMC5, SMC6, UIMC1, MPG, TDP2, USP47, CUL4B, OGG1, |
| | | BARD1 |
| GO: 0006281~DNA | 49 | RAD51C, USP3, MGMT, HUS1, INO80, MUM1, HSPA1A, CEP164, TRRAP, |
| repair | | STUB1, RPA1, CHD1L, NSMCE1, FBXO6, ACTL6A, ERCC3, INO80D, |
| | | INO80C, INO80B, POLK, MSH6, UBE2A, NUDT1, PDS5B, TAOK1, MSH2, |
| | | NEIL2, CINP, APTX, GTF2H3, CDK9, MBD4, POLB, ATM, RAD50, CDK2, |
| | | RAD1, XPA, RECQL, NABP1, XPC, CSNK1D, BTG2, CSNK1E, DDB2, |
| | | UBE2W, PSME4, PARP4, OGG1 |
| Enrichment Score: | | |
| 4.995051000869093 | | |
| Nucleotide-binding | 267 | RAD51C, DYNC1LI2, ADCY7, ATP2B4, PSKH1, CLK2, CUL9, CLK4, ILK, |
| | | DHX34, VPS4A, TLK1, DDX10, MAP2K7, PAN3, TNIK, ROCK1, ROCK2, |
| | | PIM1, UBE2J1, UBE2J2, MARK2, MAPK1, GLUL, NME3, RAB18, MAPK6, |
| | | DHX29, CAMK4, ATP2C1, RFC2, MAPK8, ARL8B, CLCN3, PFKFB3, FARS2, |
| | | UBA5, WARS2, HSPA1A, OAS1, MYO9B, ARF6, OAS2, MTIF2, NAGK, |
| | | UHMK1, MOV10, KRAS, VRK3, RAC1, ZAP70, NAT10, KIF3B, MOCS2, |
| | | TAOK1, MAP2K4, ATP11A, OXSR1, ATM, ATP13A1, UBA3, ARF4, RIT1, |
| | | SPG7, ABCF3, RAB5B, RAB5C, FASTK, UBE2G1, GTPBP10, GNL3L, HELZ, |
| | | PMVK, LATS1, ATAD3A, LONP1, ARL5A, DYNC1H1, TOP2B, NT5C, |
| | | SRPK2, RAP2C, PIK3C2A, PI4KB, DGUOK, GMPS, SRPK1, RAD50, TTF2, |
| | | CBWD2, TRAP1, RIPK1, RRM1, RAB5A, FARSB, CDK11B, MAP3K14, |
| | | ARL4C, MAP3K13, ARL4A, WRNIP1, MKNK2, KTI12, SNRK, DGKE, |
| | | STK40, DDX19A, TYW1, RAB11B, DHX16, PRKAA1, CERK, ACSL4, |
| | | ACSL3, SPATA5, ACSL5, CSNK1A1, DNM3, MAT2A, PDK3, DGKH, PCK2, |
| | | NRAS, RPS6KA3, RAB30, CSNK1D, CSNK1E, MAPK13, RAB35, GSK3B, |
| | | ARAF, DGKZ, GNA13, NT5C3A, IDE, PASK, HBS1L, INO80, DSTYK, |
| | | PI4K2B, NLRC5, PRKAR2A, IDH3G, DDX23, AAK1, ORC4, RALB, |
| | | PRKACB, SAR1B, MX1, MX2, MATK, PDXK, CSNK1G2, EFTUD2, RIPK2, |
| | | CSNK1G3, YARS2, SMARCA2, PCCB, GBP3, PRPS2, PRPS1, GPN3, MVD, |
| | | STK10, MAPKAPK5, MAP4K1, MAPKAPK2, RRAGC, IRAK4, UBE2D4, |
| | | GFM2, FICD, GFM1, DDX42, NIN, RYK, MYO1G, ABCB7, RAB33A, |
| | | RAB33B, PSMC5, PSMC2, ULK3, DYRK1A, GTF2F2, DDX50, RHOT1, |
| | | RHOT2, ABL2, DDX51, UBE2E1, ATL3, PRKAG2, DICER1, HINT2, PPIP5K2, |
| | | SKIV2L2, SLFN5, PIP5K1A, GSS, SLK, CDK12, MKKS, ATP8B2, CDK10, |
| | | TUBA1A, RHOF, CHUK, TUBA1C, CDK13, AKT2, ARL2, IRAK1, UBE2A, |
| | | PFKL, PRKCI, PKN2, PRKCH, CDK9, CDK7, PRKCD, CDK2, ARL3, PRKCB, |
| | | GAK, MCM6, TOR2A, PANK4, RECQL, PANK2, HIPK1, HIPK2, UBE2W, |
| | | PRKD3, SPAST, NKIRAS2, BTAF1, PGS1, UBE2Z, DCK, UBE2R2, N4BP2, |
| | | MTHFS, CHD9, CHD7, CHD1L, MAP3K3, MAP3K1, UCK1, HSPA4, ERCC3, |
| | | EHD1, CHD6, EHD4, MSH6, GIMAP5, MSH2, SMC5, SMC6, RAF1, DRG1, |
| | | DRG2, SMC4, GIMAP1, JAK2 |
| SM00220: S_TKc | 74 | PASK, DSTYK, LATS1, PSKH1, SLK, CLK2, AAK1, CLK4, CDK12, TLK1, |
| | | CDK10, PRKACB, MAP2K7, CHUK, CDK13, AKT2, SRPK2, IRAK1, TNIK, |
| | | ROCK1, CSNK1G2, ROCK2, PRKCI, PKN2, PIM1, PRKCH, CDK9, CDK7, |
| | | PRKCD, SRPK1, CDK2, MARK2, GAK, PRKCB, MAPK1, MAPK6, CAMK4, |
| | | HIPK1, RIPK1, HIPK2, RIPK2, CDK11B, MAPK8, CSNK1G3, MAP3K14, |
| | | MAP3K13, PRKD3, STK10, MAPKAPK5, MKNK2, MAP4K1, MAPKAPK2, |
| | | UHMK1, TRIB2, IRAK4, MAP3K3, VRK3, SNRK, STK40, MAP3K1, |
| | | PRKAA1, CSNK1A1, TAOK1, MAP2K4, RAF1, OXSR1, RPS6KA3, CSNK1D, |
| | | CSNK1E, MAPK13, GSK3B, ARAF, ULK3, DYRK1A |
| ATP-binding | 210 | RAD51C, DYNC1LI2, ADCY7, ATP2B4, PSKH1, CLK2, CUL9, CLK4, ILK, |
| | | DHX34, VPS4A, TLK1, DDX10, MAP2K7, PAN3, TNIK, ROCK1, ROCK2, |
| | | UBE2J1, PIM1, UBE2J2, MARK2, MAPK1, GLUL, NME3, MAPK6, DHX29, |
| | | CAMK4, RFC2, ATP2C1, MAPK8, CLCN3, PFKFB3, FARS2, UBA5, WARS2, |
| | | HSPA1A, OAS1, MYO9B, OAS2, NAGK, UHMK1, MOV10, KRAS, ZAP70, |
| | | NAT10, KIF3B, TAOK1, MAP2K4, ATP11A, OXSR1, ATM, ATP13A1, UBA3, |
| | | ABCF3, SPG7, FASTK, UBE2G1, HELZ, PMVK, LATS1, LONP1, ATAD3A, |
| | | DYNC1H1, TOP2B, SRPK2, PIK3C2A, DGUOK, PI4KB, GMPS, SRPK1, |
| | | RAD50, TTF2, CBWD2, TRAP1, RIPK1, RRM1, FARSB, CDK11B, MAP3K14, |
| | | MAP3K13, WRNIP1, MKNK2, SNRK, DGKE, STK40, KTI12, DDX19A, |
| | | DHX16, PRKAA1, CERK, ACSL4, ACSL3, SPATA5, ACSL5, CSNK1A1, |
| | | MAT2A, PDK3, DGKH, RPS6KA3, CSNK1D, CSNK1E, MAPK13, GSK3B, |
| | | ARAF, DGKZ, PASK, IDE, DSTYK, INO80, PI4K2B, NLRC5, IDH3G, DDX23, |
| | | AAK1, ORC4, PRKACB, MATK, PDXK, CSNK1G2, RIPK2, CSNK1G3, |
| | | YARS2, PCCB, SMARCA2, PRPS2, PRPS1, MVD, STK10, MAPKAPK5, |
| | | MAP4K1, MAPKAPK2, IRAK4, UBE2D4, FICD, DDX42, RYK, MYO1G, |
| | | ABCB7, PSMC5, PSMC2, ULK3, DYRK1A, GTF2F2, DDX50, ABL2, DDX51, |
| | | UBE2E1, DICER1, PRKAG2, PPIP5K2, SKIV2L2, SLFN5, PIP5K1A, GSS, |
| | | SLK, CDK12, MKKS, ATP8B2, CDK10, CHUK, CDK13, AKT2, IRAK1, |
| | | UBE2A, PFKL, PRKCI, PKN2, PRKCH, CDK9, CDK7, PRKCD, CDK2, |
| | | PRKCB, GAK, MCM6, TOR2A, PANK4, RECQL, PANK2, HIPK1, HIPK2, |
| | | UBE2W, PRKD3, SPAST, BTAF1, PGS1, UBE2Z, DCK, UBE2R2, N4BP2, |
| | | CHD9, MTHFS, CHD1L, CHD7, MAP3K3, MAP3K1, UCK1, HSPA4, CHD6, |
| | | ERCC3, EHD1, EHD4, MSH6, MSH2, SMC5, SMC6, RAF1, SMC4, JAK2 |
| IPR008271: Serine/ | 66 | PASK, DSTYK, LATS1, PSKH1, SLK, CLK2, AAK1, CLK4, CDK12, TLK1, |
| threonine-protein | | CDK10, PRKACB, MAP2K7, CHUK, CDK13, AKT2, SRPK2, MAKI, TNIK, |
| kinase, active site | | ROCK1, CSNK1G2, ROCK2, PRKCI, PKN2, PIM1, PRKCH, CDK9, CDK7, |
| | | PRKCD, SRPK1, CDK2, MARK2, GAK, PRKCB, MAPK1, MAPK6, CAMK4, |
| | | HIPK1, RIPK1, HIPK2, RIPK2, CDK11B, MAPK8, CSNK1G3, MAP3K14, |
| | | MAP3K13, PRKD3, MAPKAPK5, STK10, MKNK2, MAPKAPK2, SNRK, |
| | | STK40, MAP3K1, PRKAA1, CSNK1A1, TAOK1, MAP2K4, RAF1, RPS6KA3, |
| | | CSNK1D, CSNK1E, GSK3B, ARAF, ULK3, DYRK1A |
| Serine/threonine- | 75 | FASTK, PASK, DSTYK, LATS1, PSKH1, SLK, CLK2, AAK1, CLK4, ILK, |
| protein kinase | | CDK12, TLK1, CDK10, PRKACB, MAP2K7, CHUK, CDK13, AKT2, SRPK2, |
| | | IRAK1, TNIK, ROCK1, CSNK1G2, ROCK2, PRKCI, PKN2, PIM1, PRKCH, |
| | | CDK9, CDK7, PRKCD, SRPK1, CDK2, MARK2, GAK, PRKCB, MAPK1, |
| | | MAPK6, CAMK4, HIPK1, RIPK1, HIPK2, RIPK2, CDK11B, MAPK8, |
| | | CSNK1G3, MAP3K14, MAP3K13, PRKD3, MAPKAPK5, STK10, MKNK2, |
| | | MAP4K1, MAPKAPK2, UHMK1, IRAK4, MAP3K3, SNRK, STK40, MAP3K1, |
| | | PRKAA1, CSNK1A1, TAOK1, MAP2K4, RAF1, OXSR1, ATM, RPS6KA3, |
| | | CSNK1D, CSNK1E, MAPK13, GSK3B, ARAF, ULK3, DYRK1A |
| nucleotide | 156 | RAD51C, DYNC1LI2, PASK, INO80, DSTYK, NLRC5, PSKH1, IDH3G, |
| phosphate-binding | | DDX23, CLK2, CUL9, AAK1, CLK4, ILK, DHX34, ORC4, VPS4A, TLK1, |
| region: ATP | | PRKACB, DDX10, MAP2K7, MATK, TNIK, PDXK, ROCK1, CSNK1G2, |
| | | ROCK2, PIM1, MARK2, MAPK1, MAPK6, DHX29, CAMK4, RFC2, RIPK2, |
| | | MAPK8, CSNK1G3, SMARCA2, PRPS2, PRPS1, CLCN3, PFKFB3, |
| | | MAPKAPK5, STK10, MAP4K1, MYO9B, MAPKAPK2, NAGK, UHMK1, |
| | | IRAK4, MOV10, VRK3, ZAP70, NAT10, DDX42, KIF3B, RYK, TAOK1, |
| | | MAP2K4, OXSR1, ABCB7, PSMC5, PSMC2, UBA3, GTF2F2, DYRK1A, |
| | | ULK3, DDX50, ABL2, DDX51, SPG7, DICER1, HELZ, SKIV2L2, SLFN5, |
| | | PMVK, LATS1, GSS, ATAD3A, LONP1, SLK, CDK12, MKKS, CDK10, |
| | | TOP2B, DYNC1H1, CHUK, CDK13, AKT2, SRPK2, IRAK1, PFKL, PRKCI, |
| | | PKN2, PRKCH, CDK9, DGUOK, CDK7, GMPS, PRKCD, SRPK1, CDK2, |
| | | RAD50, TTF2, PRKCB, MCM6, CBWD2, TOR2A, RECQL, HIPK1, RIPK1, |
| | | HIPK2, CDK11B, MAP3K14, MAP3K13, PRKD3, SPAST, BTAF1, PGS1, |
| | | WRNIP1, MKNK2, DCK, N4BP2, CHD9, MTHFS, CHD1L, CHD7, KTI12, |
| | | STK40, MAP3K3, SNRK, DDX19A, MAP3K1, DHX16, PRKAA1, UCK1, |
| | | CHD6, EHD1, ERCC3, EHD4, CSNK1A1, MSH6, MAT2A, MSH2, PDK3, |
| | | SMC5, SMC6, RAF1, SMC4, RPS6KA3, CSNK1D, CSNK1E, MAPK13, |
| | | GSK3B, ARAF, JAK2 |
| Kinase | 120 | PASK, NELL2, DSTYK, PI4K2B, PRKAR2A, PSKH1, CLK2, AAK1, CLK4, |
| | | ILK, TLK1, PRKACB, MAP2K7, MATK, TNIK, PDXK, ROCK1, CSNK1G2, |
| | | ROCK2, PRKAB1, PIM1, PKIA, MARK2, WDR83, MAPK1, NME3, CAMK4, |
| | | MAPK6, RIPK2, MAPK8, CSNK1G3, PRPS2, PRPS1, PHKA2, PFKFB3, |
| | | STK10, MAPKAPK5, MAP4K1, AKAP10, MAPKAPK2, NAGK, UHMK1, |
| | | IRAK4, VRK3, PRKRA, ZAP70, TAOK1, RYK, CINP, MAP2K4, FN3KRP, |
| | | OXSR1, ATM, CDKN1B, DYRK1A, ULK3, HGS, ABL2, FASTK, PRKAG2, |
| | | PPIP5K2, PIP5K1A, PMVK, LATS1, SLK, CDK12, CDK10, CHUK, CDK13, |
| | | AKT2, SRPK2, IRAK1, PFKL, PIK3C2A, PKN2, PRKCI, CDK9, PRKCH, |
| | | DGUOK, PI4KB, CDK7, PRKCD, SRPK1, CDK2, GAK, PRKCB, PANK4, |
| | | PANK2, HIPK1, RIPK1, HIPK2, CDK11B, MAP3K14, MAP3K13, PRKD3, |
| | | MOB1B, DCK, MKNK2, MAP3K3, STK40, DGKE, SNRK, MAP3K1, UCK1, |
| | | PRKAA1, CERK, PIK3R1, CSNK1A1, PDK3, RAF1, DGKH, PCK2, RPS6KA3, |
| | | CSNK1D, CSNK1E, MAPK13, GSK3B, ARAF, DGKZ, JAK2 |
| binding site: ATP | 94 | SPG7, PASK, DSTYK, PMVK, LATS1, GSS, PSKH1, SLK, CLK2, AAK1, |
| | | CLK4, ILK, CDK12, TLK1, CDK10, PRKACB, MAP2K7, CHUK, CDK13, |
| | | AKT2, MATK, SRPK2, IRAK1, TNIK, ROCK1, CSNK1G2, ROCK2, PIM1, |
| | | PKN2, PRKCI, CDK9, PRKCH, CDK7, PRKCD, SRPK1, CDK2, MARK2, |
| | | PRKCB, TRAP1, MAPK1, NME3, HIPK1, CAMK4, MAPK6, RIPK1, HIPK2, |
| | | RIPK2, CDK11B, MAPK8, CSNK1G3, YARS2, MAP3K14, MAP3K13, PRKD3, |
| | | PRPS2, PRPS1, STK10, MAPKAPK5, MKNK2, MAP4K1, UBA5, MAPKAPK2, |
| | | NAGK, UHMK1, IRAK4, MTHFS, MAP3K3, VRK3, SNRK, STK40, MAP3K1, |
| | | ZAP70, PRKAA1, UCK1, EHD1, EHD4, CSNK1A1, MAT2A, RYK, TAOK1, |
| | | PDK3, MAP2K4, RAF1, OXSR1, RPS6KA3, CSNK1D, CSNK1E, MAPK13, |
| | | GSK3B, ARAF, ULK3, DYRK1A, JAK2, ABL2 |
| GO: 0004674~protein | 72 | CCNT2, FASTK, PASK, CCNT1, DSTYK, LATS1, PSKH1, SLK, CLK2, |
| serine/threonine | | AAK1, CLK4, ILK, TLK1, CDK10, PRKACB, CDK13, AKT2, SRPK2, IRAK1, |
| kinase activity | | TNIK, ROCK1, CSNK1G2, ROCK2, PRKCI, PKN2, PIM1, PRKCH, CDK9, |
| | | CDK7, PRKCD, SRPK1, CDK2, MARK2, GAK, PRKCB, MAPK1, MAPK6, |
| | | HIPK1, RIPK1, HIPK2, RIPK2, CDK11B, MAPK8, CSNK1G3, MAP3K14, |
| | | MAP3K13, MAPKAPK5, STK10, MKNK2, MAP4K1, MAPKAPK2, UHMK1, |
| | | IRAK4, VRK3, SNRK, STK40, MAP3K1, PRKAA1, CSNK1A1, TAOK1, |
| | | PDK3, RAF1, OXSR1, ATM, RPS6KA3, CSNK1D, CSNK1E, MAPK13, |
| | | GSK3B, ARAF, ULK3, DYRK1A |
| GO: 0006468~protein | 83 | CCNT2, FASTK, PRKAG2, PASK, CCNT1, LATS1, ST3GAL1, PSKH1, CLK2, |
| phosphorylation | | AAK1, ILK, TLK1, CDK10, PRKACB, CHUK, MATK, SRPK2, IRAK1, |
| | | CTBP1, PAN3, TNIK, ROCK1, CSNK1G2, ROCK2, CCNH, PIM1, PRKCI, |
| | | PKN2, PRKAB1, PRKCH, CDK9, DGUOK, CDK7, PRKCD, SRPK1, MARK2, |
| | | GAK, HCST, PRKCB, MAPK1, MAPK6, CAMK4, HIPK1, HIPK2, CDK11B, |
| | | MAPK8, MAP3K13, PRKD3, PHKA2, STK10, HUS1, MKNK2, MAP4K1, |
| | | MAPKAPK2, NPRL2, TRIB2, SNRK, STK40, MORC3, MAP3K1, PRKRA, |
| | | PPP3CB, ZAP70, PRKAA1, ERCC3, PIK3R1, CSNK1A1, FYB, TAOK1, RYK, |
| | | CREB1, RAF1, BIRC6, OXSR1, ATM, GMFB, RPS6KA3, CSNK1D, CSNK1E, |
| | | RSRC1, GSK3B, DYRK1A, JAK2 |
| active site: Proton | 105 | CNDP2, PASK, IDE, DSTYK, PSKH1, CLK2, AAK1, CLK4, TLK1, PRKACB, |
| acceptor | | MAP2K7, MATK, TNIK, ROCK1, CSNK1G2, ROCK2, PIM1, MARK2, |
| | | MAPK1, CAMK4, MAPK6, RIPK2, KDSR, MAPK8, CSNK1G3, MDH1, |
| | | HSD17B11, ME2, STK10, MAPKAPK5, MAP4K1, MAPKAPK2, ACAT2, |
| | | UHMK1, IRAK4, GALM, VRK3, IVD, ZAP70, TAOK1, RYK, MAP2K4, |
| | | OXSR1, ULK3, DYRK1A, ABL2, DCXR, HTATIP2, DHRSX, LATS1, SLK, |
| | | CDK12, CDK10, CHUK, CDK13, AKT2, IRAK1, SRPK2, PFKL, PKN2, |
| | | PRKCI, CDK9, PRKCH, CDK7, PRKCD, CDK2, SRPK1, GAK, DHRS7, |
| | | PRKCB, G6PD, HIPK1, RIPK1, TGDS, HIPK2, RRM1, TXNRD1, CDK11B, |
| | | MAP3K14, MAP3K13, PRKD3, ALDH9A1, MKNK2, ERI3, MAP3K3, STK40, |
| | | SNRK, MAP3K1, PITRM1, PRKAA1, HSD17B8, CSNK1A1, RAF1, SIRT6, |
| | | SIRT7, SIRT2, SDHA, RPS6KA3, CSNK1D, CSNK1E, TDP2, MAPK13, |
| | | GSK3B, ARAF, JAK2 |
| GO: 0004672~protein | 67 | FASTK, PASK, CLK2, AAK1, ILK, CDK12, CDK10, MAP2K7, CHUK, |
| kinase activity | | CDK13, AKT2, SRPK2, IRAK1, TNIK, PAN3, ROCK1, CSNK1G2, PRKCI, |
| | | PKN2, PRKAB1, GTF2H3, PRKCH, CDK9, CDK7, PRKCD, SRPK1, CDK2, |
| | | MARK2, GAK, PRKCB, CAMK4, HIPK1, RIPK1, HIPK2, CDK11B, |
| | | CSNK1G3, MAP3K14, CCL3, MAPKAPK5, MKNK2, MAP4K1, MAPKAPK2, |
| | | NPRL2, TRIB2, IRAK4, VRK3, SNRK, MAP3K3, MAP3K1, PRKAA1, ERCC3, |
| | | CSNK1A1, TAOK1, RYK, PDK3, MAP2K4, RAF1, RPS6KA3, CSNK1D, |
| | | CSNK1E, MAPK13, GSK3B, ARAF, ULK3, DYRK1A, JAK2, ABL2 |
| domain: Protein | 79 | PASK, DSTYK, LATS1, PSKH1, SLK, CLK2, AAK1, CLK4, ILK, CDK12, |
| kinase | | TLK1, CDK10, PRKACB, MAP2K7, CHUK, CDK13, AKT2, MATK, SRPK2, |
| | | IRAK1, PAN3, TNIK, ROCK1, CSNK1G2, ROCK2, PIM1, PRKCI, PKN2, |
| | | PRKCH, CDK9, CDK7, PRKCD, SRPK1, CDK2, MARK2, GAK, PRKCB, |
| | | MAPK1, MAPK6, CAMK4, HIPK1, RIPK1, HIPK2, RIPK2, CDK11B, MAPK8, |
| | | CSNK1G3, MAP3K14, MAP3K13, PRKD3, STK10, MAPKAPK5, MKNK2, |
| | | MAP4K1, MAPKAPK2, UHMK1, TRIB2, IRAK4, MAP3K3, VRK3, SNRK, |
| | | STK40, MAP3K1, ZAP70, PRKAA1, CSNK1A1, TAOK1, RYK, MAP2K4, |
| | | RAF1, OXSR1, CSNK1D, CSNK1E, MAPK13, GSK3B, ARAF, ULK3, |
| | | DYRK1A, ABL2 |
| IPR000719: Protein | 81 | PASK, DSTYK, LATS1, PSKH1, SLK, CLK2, AAK1, CLK4, ILK, CDK12, |
| kinase, catalytic | | TLK1, CDK10, PRKACB, MAP2K7, CHUK, CDK13, AKT2, MATK, SRPK2, |
| domain | | IRAK1, PAN3, TNIK, ROCK1, CSNK1G2, ROCK2, PIM1, PRKCI, PKN2, |
| | | PRKCH, CDK9, CDK7, PRKCD, SRPK1, CDK2, MARK2, GAK, PRKCB, |
| | | MAPK1, CAMK4, MAPK6, HIPK1, RIPK1, HIPK2, RIPK2, CDK11B, MAPK8, |
| | | CSNK1G3, MAP3K14, MAP3K13, PRKD3, STK10, MAPKAPK5, MKNK2, |
| | | MAP4K1, MAPKAPK2, UHMK1, TRIB2, IRAK4, MAP3K3, VRK3, SNRK, |
| | | STK40, MAP3K1, ZAP70, PRKAA1, CSNK1A1, TAOK1, RYK, MAP2K4, |
| | | RAF1, OXSR1, RPS6KA3, CSNK1D, CSNK1E, MAPK13, GSK3B, ARAF, |
| | | ULK3, DYRK1A, JAK2, ABL2 |
| IPR011009: Protein | 86 | PASK, DSTYK, LATS1, PSKH1, SLK, CLK2, AAK1, CLK4, ILK, CDK12, |
| kinase-like domain | | TLK1, CDK10, PRKACB, MAP2K7, CHUK, CDK13, AKT2, MATK, SRPK2, |
| | | IRAK1, PAN3, TNIK, ROCK1, CSNK1G2, ROCK2, PIK3C2A, PIM1, PRKCI, |
| | | PKN2, CDK9, PRKCH, PI4KB, CDK7, PRKCD, SRPK1, CDK2, GAK, MARK2, |
| | | PRKCB, MAPK1, CAMK4, MAPK6, HIPK1, RIPK1, HIPK2, RIPK2, CDK11B, |
| | | MAPK8, CSNK1G3, MAP3K14, MAP3K13, PRKD3, STK10, MAPKAPK5, |
| | | MKNK2, MAP4K1, MAPKAPK2, TRRAP, UHMK1, TRIB2, IRAK4, MAP3K3, |
| | | VRK3, SNRK, STK40, MAP3K1, ZAP70, PRKAA1, CSNK1A1, TAOK1, RYK, |
| | | MAP2K4, RAF1, FN3KRP, OXSR1, ATM, RPS6KA3, CSNK1D, CSNK1E, |
| | | MAPK13, GSK3B, ARAF, ULK3, DYRK1A, JAK2, ABL2 |
| GO: 0005524~ATP | 216 | RAD51C, DYNC1LI2, ADCY7, ATP2B4, PSKH1, CLK2, CUL9, CLK4, ILK, |
| binding | | DHX34, VPS4A, TLK1, DDX10, MAP2K7, PAN3, TNIK, ROCK1, ROCK2, |
| | | UBE2J1, PIM1, UBE2J2, MARK2, MAPK1, GLUL, NME3, MAPK6, DHX29, |
| | | CAMK4, ATP2C1, RFC2, MAPK8, CLCN3, PFKFB3, FARS2, UBA5, WARS2, |
| | | HSPA1A, OAS1, MYO9B, OAS2, NAGK, UHMK1, MOV10, KRAS, VRK3, |
| | | ZAP70, NAT10, KIF3B, TAOK1, MAP2K4, ATP11A, OXSR1, ATM, |
| | | ATP13A1, UBA3, ABCF3, SPG7, FASTK, UBE2G1, HELZ, PMVK, LATS1, |
| | | LONP1, ATAD3A, DYNC1H1, TOP2B, SRPK2, PIK3C2A, DGUOK, PI4KB, |
| | | GMPS, SRPK1, RAD50, TTF2, CBWD2, TRAP1, RIPK1, RRM1, FARSB, |
| | | CDK11B, MAP3K14, MAP3K13, WRNIP1, MKNK2, SNRK, DGKE, STK40, |
| | | KTI12, DDX19A, DHX16, PRKAA1, CERK, ACSL4, ACSL3, SPATA5, |
| | | ACSL5, CSNK1A1, PDS5B, MAT2A, PDK3, DGKH, RPS6KA3, CSNK1D, |
| | | CSNK1E, MAPK13, GSK3B, ARAF, DGKZ, PASK, IDE, DSTYK, INO80, |
| | | PI4K2B, NLRC5, IDH3G, DDX23, AAK1, ORC4, PRKACB, MATK, PDXK, |
| | | CSNK1G2, PNPLA8, RIPK2, CSNK1G3, YARS2, SLFN12L, SMARCA2, |
| | | PCCB, PRPS2, PRPS1, MVD, STK10, MAPKAPK5, MAP4K1, MAPKAPK2, |
| | | IRAK4, UBE2D4, FICD, RUNX1, DDX42, RYK, MYO1G, ABCB7, PSMC5, |
| | | PSMC2, ULK3, DYRK1A, GTF2F2, DDX50, ABL2, DDX51, UBE2E1, |
| | | DICER1, PRKAG2, PPIP5K2, SKIV2L2, SLFN5, PIP5K1A, GSS, SLK, CDK12, |
| | | MKKS, ATP8B2, CDK10, CHUK, CDK13, AKT2, IRAK1, UBE2A, PFKL, |
| | | PRKCI, PKN2, PRKCH, CDK9, CDK7, PRKCD, CDK2, PRKCB, GAK, MCM6, |
| | | TOR2A, PANK4, RECQL, PANK2, HIPK1, HIPK2, UBE2W, PRKD3, SPAST, |
| | | BTAF1, PGS1, UBE2Z, DCK, TRIB2, UBE2R2, N4BP2, CHD9, MTHFS, |
| | | CHD1L, CHD7, MAP3K3, MAP3K1, UCK1, HSPA4, CHD6, ERCC3, EHD1, |
| | | EHD4, MSH6, MSH2, SMC5, SMC6, RAF1, SMC4, JAK2 |
| IPR017441: Protein | 62 | PASK, DSTYK, PSKH1, SLK, CLK2, CLK4, CDK12, TLK1, CDK10, |
| kinase, ATP binding | | PRKACB, CHUK, CDK13, MATK, AKT2, IRAK1, SRPK2, TNIK, ROCK1, |
| site | | CSNK1G2, ROCK2, PRKCI, PKN2, PIM1, PRKCH, CDK9, CDK7, PRKCD, |
| | | CDK2, SRPK1, MARK2, PRKCB, MAPK1, MAPK6, CAMK4, HIPK1, HIPK2, |
| | | CSNK1G3, MAP3K14, PRKD3, STK10, MKNK2, MAP4K1, MAPKAPK2, |
| | | SNRK, MAP3K1, ZAP70, PRKAA1, CSNK1A1, TAOK1, MAP2K4, RAF1, |
| | | OXSR1, RPS6KA3, CSNK1D, CSNK1E, MAPK13, GSK3B, ARAF, ULK3, |
| | | DYRK1A, JAK2, ABL2 |
| Enrichment Score: | | |
| 4.4354405219010475 | | |
| Mitochondrion | 173 | RAD51C, TSPO, MRPL42, CMC2, MALSUl, GFER, MPV17, TMEM11, |
| | | OGDH, CIAPIN1, FAM210A, HIBADH, MFF, IDH3G, VPS13C, CASP8AP2, |
| | | CPOX, SLC25A28, MRPL34, MRPL35, TIMMDC1, CRLS1, NUDT1, |
| | | BCL2L11, TIMM8A, NFU1, SLC25A32, GLUL, SLC25A38, YARS2, TFB1M, |
| | | PCCB, MTFMT, MPST, MRPL44, ME2, ELAC2, MRPS14, MCL1, GLUD2, |
| | | TXN2, FARS2, MRPS11, SFXN4, WARS2, AKAP10, OAS1, CHCHD4, OAS2, |
| | | RBFA, PIN4, AGMAT, MTIF2, SDHAF1, FAM65B, SLC11A2, FIS1, GFM2, |
| | | C12ORF10, IVD, GFM1, BLOC1S1, IDH2, MRPL55, LIAS, TRAF3, FH, |
| | | MRPS23, MRPS25, C21ORF33, MPC1, MPC2, GLOD4, MRRF, ABCB7, PPIF, |
| | | TEFM, NDUFV3, METTL12, SYNE2, BBC3, NDUFV2, RHOT1, RHOT2, |
| | | SLC25A16, C19ORF12, PHYKPL, METTL17, NDUFAF4, SPG7, COX11, |
| | | UQCRC1, FASTK, HINT2, BNIP3, UQCRFS1, ARL2BP, ACOT9, LONP1, |
| | | DNAJC15, ATAD3A, PARL, DNAJC11, ATP5H, NDUFS1, ARL2, SQRDL, |
| | | AIFM1, DGUOK, PI4KB, ECSIT, RHBDD1, NDUFA10, MRPS2, TIMM22, |
| | | IMMP1L, HAGH, TRAP1, PANK2, MRPS9, BNIP1, TCHP, ATPAF1, |
| | | C7ORF73, GLRX5, PGS1, BCAT2, NDUFB7, ETHE1, RSAD2, QTRT1, HSCB, |
| | | TACO1, GLRX2, NUDT9, BCL2, PITRM1, MRPL16, PYCARD, XAF1, |
| | | ACSL4, LACTB, PDHX, ACSL3, SPATA5, SCO2, ACSL5, ETFA, C14ORF119, |
| | | HSD17B8, ECI1, DLST, ALKBH7, ECI2, GIMAP5, IMMT, NDUFA9, PDK3, |
| | | RAF1, BAD, PCK2, IFIT3, SDHA, MPG, APOPT1, SDHC, TSFM, MTFP1, |
| | | BNIP3L, NLN, OGG1, SCP2, SLC25A53 |
| Transit peptide | 87 | COX11, MRPL42, UQCRC1, HINT2, OGDH, UQCRFS1, HIBADH, ACOT9, |
| | | LONP1, IDH3G, PARL, CPDX, MRPL34, NDUFS1, MRPL35, SQRDL, |
| | | NUDT1, AIFM1, DGUOK, NDUFA10, ECSIT, HAGH, TRAP1, NFUl, PANK2, |
| | | MRPS9, YARS2, TFB1M, ATPAF1, PCCB, MTFMT, MRPL44, PGS1, GLRX5, |
| | | BCAT2, ELAC2, ME2, TXN2, GLUD2, FARS2, ETHEL MRPS11, WARS2, |
| | | AKAP10, RBFA, MTIF2, AGMAT, HSCB, GLRX2, GFM2, C12ORF10, |
| | | NUDT9, IVD, GFM1, MRPL16, PITRM1, IDH2, MRPL55, LIAS, LACTB, |
| | | PDHX, SCO2, ETFA, FH, ECI1, ECI2, DLST, ALKBH7, NDUFA9, IMMT, |
| | | PDK3, C21ORF33, PCK2, MRRF, ABCB7, NDUFV3, TEFM, PPIF, SDHA, |
| | | METTL12, MPG, APOPT1, SDHC, TSFM, NDUFV2, NLN, METTL17 |
| transit | 80 | COX11, MRPL42, UQCRC1, HINT2, OGDH, UQCRFS1, HIBADH, ACOT9, |
| peptide: Mitochondrion | | LONP1, IDH3G, PARL, CPDX, MRPL34, NDUFS1, MRPL35, SQRDL, AIFM1, |
| | | DGUOK, NDUFA10, ECSIT, HAGH, TRAP1, NFUl, PANK2, MRPS9, YARS2, |
| | | TFB1M, ATPAF1, PCCB, MTFMT, MRPL44, PGS1, BCAT2, ME2, TXN2, |
| | | GLUD2, FARS2, ETHE1, MRPS11, WARS2, AKAP10, RBFA, MTIF2, |
| | | AGMAT, HSCB, GLRX2, GFM2, C1O0RF10, NUDT9, IVD, GFM1, MRPL16, |
| | | PITRM1, IDH2, MRPL55, LIAS, LACTB, PDHX, SCO2, ETFA, FH, ECI1, |
| | | DLST, ECI2, NDUFA9, PDK3, C21ORF33, PCK2, MRRF, ABCB7, NDUFV3, |
| | | PPIF, SDHA, METTL12, TXNDC12, SDHC, TSFM, NDUFV2, NLN, METTL17 |
| GO: 0005759~mito- | 57 | FASTK, MALSUl, OGDH, HIBADH, ARL2BP, ACOT9, GPX1, LONP1, |
| chondrial matrix | | IDH3G, NDUFS1, ARL2, NUDT1, DGUOK, NDUFA10, HAGH, TRAP1, |
| | | YARS2, TFB1M, PCCB, GLRX5, ME2, ELAC2, BCAT2, MCL1, TXN2, |
| | | ETHE1, FARS2, WARS2, PIN4, SDHAF1, GLRX2, MTHFS, GFM2, NUDT9, |
| | | IVD, GFM1, PITRM1, BLOC1S1, IDH2, LIAS, PDHX, SCO2, ETFA, |
| | | HSD17B8, FH, ECI1, ALKBH7, DLST, NDUFA9, CREB1, PDK3, MRRF, |
| | | PCK2, TEFM, PPIF, TSFM, PHYKPL |
| Enrichment Score: | | |
| 4.367762624189855 | | |
| domain: MBD | 8 | SETDB1, MECP2, MBD6, MBD5, MBD4, BAZ2B, MBD1, BAZ2A |
| SM00391: MBD | 8 | SETDB1, MECP2, MBD6, MBD5, MBD4, BAZ2B, MBD1, BAZ2A |
| IPR016177: DNA- | 8 | SETDB1, MECP2, MBD6, MBD5, MBD4, BAZ2B, MBD1, BAZ2A |
| binding, integrase- | | |
| type | | |
| IPR001739: Methyl- | 8 | SETDB1, MECP2, MBD6, MBD5, MBD4, BAZ2B, MBD1, BAZ2A |
| CpG DNA binding | | |
| Enrichment Score: | | |
| 4.255632723903859 | | |
| SM00320: WD40 | 55 | COPA, SEC31B, SEC31A, STRN, TSSC1, WDR74, SHKBP1, WDR77, ZNF106, |
| | | MLST8, NSMAF, TBL1XR1, ELP2, RBBP4, GNB1L, STRN3, TLE3, PRPF4, |
| | | ARPC1A, WDR48, WDR83, EML3, SMU1, MED16, NOL10, DDB2, THOC6, |
| | | DYNC1I2, WDR45, WDR60, LRBA, WDR45B, PRPF19, PHIP, FBXW7, |
| | | NUP214, WDR54, FBXW5, FBXW2, WDR12, PAFAH1B1, FBXW11, |
| | | PPP2R2D, GEMIN5, WDTC1, WDR5, WDR6, WIPI2, PWP2, WSB1, POC1B, |
| | | WDR26, WRAP73, TBL1X, CSTF1 |
| IPR017986: WD40- | 62 | COPA, TAF1C, SEC31B, SEC31A, KNTC1, STRN, TSSC1, WDR74, SHKBP1, |
| repeat-containing | | WDR77, ZNF106, MLST8, NSMAF, ITFG2, TBL1XR1, ELP2, RBBP4, GNB1L, |
| domain | | STRN3, ANAPC4, TLE3, VPS41, PRPF4, ARPC1A, WDR48, WDR83, EML3, |
| | | SMU1, MED16, DDB2, NOL10, THOC6, NOL11, DYNC1I2, WDR45, LRBA, |
| | | WDR60, SF3B3, WDR45B, PRPF19, PHIP, FBXW7, WDR54, FBXW5, |
| | | WDR12, FBXW2, PAFAH1B1, FBXW11, VPS39, PPP2R2D, GEMIN5, |
| | | WDTC1, WDR5, WDR6, WIPI2, PWP2, WSB1, POC1B, WDR26, WRAP73, |
| | | TBL1X, CSTF1 |
| repeat: WD 3 | 54 | COPA, SEC31B, SEC31A, STRN, TSSC1, WDR74, SHKBP1, WDR77, ZNF106, |
| | | MLST8, NSMAF, TBL1XR1, ELP2, RBBP4, GNB1L, STRN3, TLE3, HERC2, |
| | | PRPF4, ARPC1A, WDR48, WDR83, EML3, SMU1, MED16, NOL10, DDB2, |
| | | THOC6, DYNC1I2, WDR45, WDR60, LRBA, PRPF19, PHIP, FBXW7, WDR54, |
| | | FBXW5, FBXW2, WDR12, PAFAH1B1, FBXW11, PPP2R2D, GEMIN5, |
| | | WDTC1, WDR5, WDR6, WIPI2, PWP2, WSB1, POC1B, WDR26, WRAP73, |
| | | TBL1X, CSTF1 |
| IPR001680: WD40 | 55 | COPA, SEC31B, SEC31A, STRN, TSSC1, WDR74, SHKBP1, WDR77, ZNF106, |
| repeat | | MLST8, NSMAF, TBL1XR1, ELP2, RBBP4, GNB1L, STRN3, TLE3, PRPF4, |
| | | ARPC1A, WDR48, WDR83, EML3, SMU1, MED16, NOL10, DDB2, THOC6, |
| | | DYNC1I2, WDR45, WDR60, LRBA, WDR45B, PRPF19, PHIP, FBXW7, |
| | | NUP214, WDR54, FBXW5, FBXW2, WDR12, PAFAH1B1, FBXW11, |
| | | PPP2R2D, GEMIN5, WDTC1, WDR5, WDR6, WIPI2, PWP2, WSB1, POC1B, |
| | | WDR26, WRAP73, TBL1X, CSTF1 |
| repeat: WD 1 | 55 | COPA, SEC31B, SEC31A, STRN, TSSC1, WDR74, SHKBP1, WDR77, ZNF106, |
| | | MLST8, NSMAF, TBL1XR1, ELP2, RBBP4, GNB1L, STRN3, TLE3, HERC2, |
| | | PRPF4, ARPC1A, WDR48, WDR83, EML3, SMU1, MED16, NOL10, DDB2, |
| | | THOC6, DYNC1I2, WDR45, WDR60, LRBA, WDR45B, PRPF19, PHIP, |
| | | FBXW7, WDR54, FBXW5, FBXW2, WDR12, PAFAH1B1, FBXW11, |
| | | PPP2R2D, GEMIN5, WDTC1, WDR5, WDR6, WIPI2, PWP2, WSB1, POC1B, |
| | | WDR26, WRAP73, TBL1X, CSTF1 |
| repeat: WD 2 | 55 | COPA, SEC31B, SEC31A, STRN, TSSC1, WDR74, SHKBP1, WDR77, ZNF106, |
| | | MLST8, NSMAF, TBL1XR1, ELP2, RBBP4, GNB1L, STRN3, TLE3, HERC2, |
| | | PRPF4, ARPC1A, WDR48, WDR83, EML3, SMU1, MED16, NOL10, DDB2, |
| | | THOC6, DYNC1I2, WDR45, WDR60, LRBA, WDR45B, PRPF19, PHIP, |
| | | FBXW7, WDR54, FBXW5, FBXW2, WDR12, PAFAH1B1, FBXW11, |
| | | PPP2R2D, GEMIN5, WDTC1, WDR5, WDR6, WIPI2, PWP2, WSB1, POC1B, |
| | | WDR26, WRAP73, TBL1X, CSTF1 |
| repeat: WD 4 | 51 | COPA, SEC31B, SEC31A, STRN, TSSC1, SHKBP1, WDR74, WDR77, ZNF106, |
| | | MLST8, NSMAF, TBL1XR1, ELP2, RBBP4, GNB1L, STRN3, TLE3, HERC2, |
| | | PRPF4, ARPC1A, WDR48, WDR83, EML3, SMU1, MED16, NOL10, DDB2, |
| | | THOC6, DYNC1I2, WDR60, LRBA, PRPF19, PHIP, FBXW7, WDR54, |
| | | FBXW2, WDR12, PAFAH1B1, FBXW11, PPP2R2D, GEMIN5, WDTC1, |
| | | WDR5, WDR6, PWP2, WSB1, POC1B, WDR26, WRAP73, TBL1X, CSTF1 |
| WD repeat | 54 | COPA, SEC31B, SEC31A, STRN, TSSC1, WDR74, SHKBP1, WDR77, ZNF106, |
| | | MLST8, NSMAF, TBL1XR1, ELP2, RBBP4, GNB1L, STRN3, TLE3, PRPF4, |
| | | ARPC1A, WDR48, WDR83, EML3, SMU1, MED16, NOL10, DDB2, THOC6, |
| | | DYNC1I2, WDR45, WDR60, LRBA, WDR45B, PRPF19, PHIP, FBXW7, |
| | | WDR54, FBXW5, FBXW2, WDR12, PAFAH1B1, FBXW11, PPP2R2D, |
| | | GEMIN5, WDTC1, WDR5, WDR6, WIPI2, PWP2, WSB1, POC1B, WDR26, |
| | | WRAP73, TBL1X, CSTF1 |
| repeat: WD 6 | 42 | COPA, SEC31B, SEC31A, LRBA, STRN, WDR74, PRPF19, PHIP, FBXW7, |
| | | WDR12, ZNF106, PAFAH1B1, MLST8, NSMAF, FBXW11, PPP2R2D, |
| | | GEMIN5, TBL1XR1, ELP2, WDTC1, RBBP4, GNB1L, STRN3, WDR5, WDR6, |
| | | TLE3, HERC2, PRPF4, PWP2, WDR48, ARPC1A, WSB1, WDR83, EML3, |
| | | SMU1, POC1B, WDR26, WRAP73, THOC6, TBL1X, CSTF1, DYNC1I2 |
| repeat: WD 5 | 48 | COPA, SEC31B, SEC31A, LRBA, STRN, TSSC1, WDR74, PRPF19, SHKBP1, |
| | | PHIP, FBXW7, WDR77, WDR12, PAFAH1B1, ZNF106, MLST8, NSMAF, |
| | | FBXW11, PPP2R2D, GEMIN5, TBL1XR1, ELP2, WDTC1, RBBP4, GNB1L, |
| | | STRN3, WDR5, WDR6, TLE3, HERC2, PRPF4, PWP2, WDR48, ARPC1A, |
| | | WSB1, WDR83, EML3, SMU1, POC1B, WDR26, WRAP73, MED16, NOL10, |
| | | THOC6, DDB2, TBL1X, CSTF1, DYNC1I2 |
| IPR020472: G- | 24 | COPA, TBL1XR1, RBBP4, STRN3, WDR5, STRN, PRPF4, PWP2, WDR48, |
| protein beta WD-40 | | WDR83, PRPF19, WSB1, SMU1, FBXW7, POC1B, WDR26, FBXW2, WDR12, |
| repeat | | PAFAH1B1, MLST8, TBL1X, CSTF1, FBXW11, GEMIN5 |
| IPR015943: WD40/ | 58 | COPA, SEC31B, SEC31A, STRN, TSSC1, WDR74, SHKBP1, WDR77, ZNF106, |
| YVTN repeat-like- | | MLST8, NSMAF, TBL1XR1, ELP2, RBBP4, GNB1L, STRN3, ANAPC4, TLE3, |
| containing domain | | VPS41, PRPF4, ARPC1A, WDR48, WDR83, EML3, SMU1, MED16, NOL10, |
| | | DDB2, THOC6, DYNC1I2, WDR45, WDR60, LRBA, WDR45B, PRPF19, PHIP, |
| | | FBXW7, NUP214, WDR54, FBXW5, WDR12, FBXW2, PAFAH1B1, FBXW11, |
| | | PPP2R2D, GEMIN5, WDTC1, WDR5, WDR6, BIRC6, WIPI2, PWP2, WSB1, |
| | | POC1B, WDR26, WRAP73, TBL1X, CSTF1 |
| IPR019775: WD40 | 31 | COPA, SEC31B, STRN, TSSC1, PRPF19, PHIP, FBXW7, WDR77, FBXW2, |
| repeat, conserved | | WDR12, PAFAH1B1, MLST8, FBXW11, GEMIN5, TBL1XR1, RBBP4, |
| site | | GNB1L, WDR5, STRN3, TLE3, PRPF4, PWP2, WDR48, WDR83, WSB1, |
| | | SMU1, POC1B, THOC6, DDB2, TBL1X, CSTF1 |
| repeat: WD 7 | 26 | SEC31B, SEC31A, PHIP, PRPF19, FBXW7, WDR12, PAFAH1B1, MLST8, |
| | | FBXW11, PPP2R2D, GEMIN5, TBL1XR1, WDTC1, ELP2, WDR5, WDR6, |
| | | TLE3, PRPF4, PWP2, WDR48, WDR83, EML3, POC1B, THOC6, TBL1X, |
| | | DYNC1I2 |
| Enrichment Score: | | |
| 3.829453652617158 | | |
| Transcription | 336 | ITGB3BP, MEF2A, BBX, MED23, RORA, ZNF638, MXI1, TBPL2, BRPF1, |
| | | SIN3A, ZFP90, ZNF394, TBPL1, ZNF101, ZNF43, ZNF44, TADA2A, ZNF644, |
| | | RXRB, PCBD1, ZHX1, MECP2, MED11, HMG20B, MED13, PPARGC1A, |
| | | ZNF37A, MED19, MAPK1, PIAS4, ASCC2, HES4, MED16, MLLT10, JUN, |
| | | MED17, PRDM2, CDCA7L, PIAS1, SUDS3, CRTC3, CRTC2, ZNF131, TAF9B, |
| | | ZNF511, XAB2, NR1H2, MOV10, TCF20, LEO1, TCF3, PLAGL2, IKZF5, |
| | | ASXL2, TCF7, ESRRA, ZNF529, IKZF2, NRBF2, KLF13, TP53BP1, ZNF121, |
| | | KLF10, CREBBP, RYBP, ZBTB40, SMAD3, PMF1, RNF4, DMTF1, PPRC1, |
| | | JAZFL HOPX, KAT6B, RERE, NCOR2, NKAP, CCNT2, CREBRF, TAF1B, |
| | | TAF1C, ZNF292, ELF2, BACH2, ZNF534, EZH1, CCNT1, COPRS, ZNF675, |
| | | ZEB1, RFXANK, DAXX, ZBTB38, DNAJC17, MBTD1, ASH2L, ZNF148, |
| | | BRD4, USP16, TWISTNB, MYB, DEDD2, ZNF493, SERTAD2, BRD8, |
| | | ATF7IP, NFKBIZ, CTBP1, BRF1, RBBP4, POLR1E, BRF2, CCNH, POLR1A, |
| | | TLE3, GTF2H3, SPEN, MBD1, GTF2B, TTF2, MXD4, TAF10, TAF13, |
| | | CHMP1A, GTF2I, MED8, ASH1L, ZNF277, NOL11, CNOT11, USP22, |
| | | ZNF746, ZNF740, MED1, ZNF276, ZNF275, ETV7, ZNF274, ZBTB10, |
| | | ZBTB11, PML, ZNF780B, ZNF780A, POLR2B, MYCBP2, STAT6, LPXN, |
| | | RB1CC1, NPAT, GATAD2A, ZSCAN25, BCL3, AGO2, THAP1, ACTL6A, |
| | | PRKAA1, HBP1, BAZ2B, ZNF268, BAZ2A, POLR3F, POLR3H, ZNF28, |
| | | PPHLN1, TRIM27, PHF10, POLR3C, ATMIN, TRIM22, POLR3E, IWS1, |
| | | ZNF664, ZNF672, BRMS1, JMJD6, YAF2, MAPK13, WHSC1L1, ZBTB2, |
| | | ZNF764, ZNF766, RALY, ZNF583, CNOT8, IL16, TBP, CBX7, TCEAL4, |
| | | GABPB1, DPY30, CASP8AP2, CGGBP1, MDFIC, TARDBP, GATA3, RELA, |
| | | ZNF7, ARID1B, TRERF1, EP300, TRIM33, KDM2A, NFE2L2, AKAP8, |
| | | NFE2L3, ZNF587, TFB1M, SMARCA2, ZNF586, CAMTA2, ZNF430, LITAF, |
| | | SETD1A, KEAP1, C14ORF166, ELK3, TRRAP, COMMD9, SRF, COMMD10, |
| | | CXXC1, PELP1, CNOT6L, RNF10, RUNX1, SETDB1, ZMYM2, TRIP4, |
| | | CREBZF, TAF6, RFX5, WDR5, ZMYM5, NR4A1, SNVV1, KAT5, MED13L, |
| | | FOXP3, ATF7IP2, UIMC1, SAFB2, SREBF2, CTR9, TEFM, ATF6, ATF5, |
| | | NRF1, PHF1, GTF2F1, GTF2F2, CPNE1, HIVEP2, HIVEP1, E2F3, E2F4, |
| | | ARID4A, GPBP1, YLPM1, FOXK2, CTCF, ZKSCAN1, CBFA2T2, PCGF5, |
| | | GTF2A1, ZNF721, INO80D, KDM5B, INO80C, KDM5C, NFX1, INO80B, |
| | | ELMSAN1, EGR1, TBL1XR1, ELP2, SSBP3, LRIF1, ELP6, ELP5, ARID5A, |
| | | ZFX, PKN2, CDK9, IRF2BP2, BANP, CDK7, FOXJ3, LPIN1, NRIP1, PRKCB, |
| | | NCOA1, NCOA2, BTG2, BPTF, HIPK1, FAM120B, HIPK2, KHSRP, |
| | | COMMD3, COMMD1, WASL, JMJD1C, DPF2, ING3, SBNO2, ING2, KMT2A, |
| | | FRYL, KMT2C, ZNF800, NFYC, NFYB, PAXBP1, CHD9, CHD7, TSPYL2, |
| | | ECD, NFATC2, ERCC3, CHD6, GTF3C1, GTF3C3, L3MBTL2, L3MBTL3, |
| | | CREB1, SIRT7, MRGBP, SIRT2, MED31, RPAP2, SP1, DR1, KDM4C, IRF3, |
| | | TBL1X, VPS25 |
| Transcription | 324 | ITGB3BP, MEF2A, BBX, MED23, RORA, ZNF638, MXIL TBPL2, BRPF1, |
| regulation | | SIN3A, ZFP90, ZNF394, TBPL1, ZNF101, ZNF43, ZNF44, TADA2A, ZNF644, |
| | | RXRB, PCBD1, ZHX1, MECP2, MED11, HMG20B, MED13, PPARGC1A, |
| | | ZNF37A, MED19, MAPK1, PIAS4, ASCC2, HES4, MED16, MLLT10, JUN, |
| | | MED17, PRDM2, CDCA7L, PIAS1, SUDS3, CRTC3, CRTC2, ZNF131, TAF9B, |
| | | ZNF511, NR1H2, MOV10, TCF20, LEO1, TCF3, PLAGL2, IKZF5, ASXL2, |
| | | TCF7, ESRRA, ZNF529, IKZF2, NRBF2, KLF13, TP53BP1, ZNF121, KLF10, |
| | | CREBBP, RYBP, ZBTB40, SMAD3, PMF1, RNF4, DMTF1, PPRC1, JAZFL |
| | | HOPX, KAT6B, RERE, NCOR2, NKAP, CCNT2, CREBRF, TAF1B, TAF1C, |
| | | ZNF292, ELF2, ZNF534, BACH2, EZH1, CCNT1, COPRS, ZNF675, ZEB1, |
| | | RFXANK, DAXX, ZBTB38, DNAJC17, MBTD1, ASH2L, ZNF148, BRD4, |
| | | USP16, MYB, DEDD2, ZNF493, SERTAD2, BRD8, ATF7IP, NFKBIZ, CTBP1, |
| | | BRF1, RBBP4, BRF2, CCNH, TLE3, GTF2H3, SPEN, MBD1, GTF2B, TTF2, |
| | | MXD4, TAF10, TAF13, CHMP1A, GTF2I, MED8, ASH1L, ZNF277, NOL11, |
| | | CNOT11, USP22, ZNF746, ZNF740, MED1, ZNF276, ZNF275, ETV7, ZNF274, |
| | | ZBTB10, ZBTB11, PML, ZNF780B, ZNF780A, MYCBP2, STAT6, LPXN, |
| | | RB1CC1, NPAT, GATAD2A, ZSCAN25, BCL3, AGO2, THAP1, ACTL6A, |
| | | PRKAA1, HBP1, BAZ2B, ZNF268, BAZ2A, ZNF28, PPHLN1, TRIM27, |
| | | PHF10, ATMIN, TRIM22, IWS1, ZNF664, ZNF672, BRMS1, JMJD6, YAF2, |
| | | MAPK13, WHSC1L1, ZBTB2, ZNF764, ZNF766, RALY, ZNF583, CNOT8, |
| | | IL16, TBP, CBX7, TCEAL4, GABPB1, DPY30, CASP8AP2, CGGBP1, MDFIC, |
| | | TARDBP, GATA3, RELA, ZNF7, ARID1B, TRERF1, EP300, TRIM33, |
| | | KDM2A, NFE2L2, AKAP8, NFE2L3, ZNF587, TFB1M, SMARCA2, ZNF586, |
| | | CAMTA2, ZNF430, LITAF, SETD1A, KEAP1, C14ORF166, ELK3, TRRAP, |
| | | COMMD9, SRF, COMMD10, CXXCL CNOT6L, RNF10, RUNX1, SETDB1, |
| | | ZMYM2, TRIP4, CREBZF, TAF6, RFX5, WDR5, ZMYM5, NR4A1, SNW1, |
| | | KAT5, MED13L, FOXP3, ATF7IP2, UIMC1, SAFB2, SREBF2, CTR9, ATF6, |
| | | TEFM, ATF5, NRF1, PHF1, GTF2F1, GTF2F2, CPNE1, HIVEP2, HIVEP1, |
| | | E2F3, E2F4, ARID4A, GPBP1, YLPM1, FOXK2, CTCF, ZKSCAN1, CBFA2T2, |
| | | PCGF5, GTF2A1, ZNF721, INO80D, KDM5B, INO80C, KDM5C, NFX1, |
| | | INO80B, ELMSAN1, EGR1, TBL1XR1, ELP2, SSBP3, LRIF1, ELP6, ELP5, |
| | | ZFX, ARID5A, PKN2, CDK9, IRF2BP2, BANP, CDK7, FOXJ3, LPIN1, NRIP1, |
| | | PRKCB, NCOA1, NCOA2, BTG2, BPTF, HIPK1, FAM120B, HIPK2, KHSRP, |
| | | COMMD3, COMMD1, WASL, JMJD1C, DPF2, ING3, SBNO2, ING2, KMT2A, |
| | | FRYL, KMT2C, ZNF800, NFYC, NFYB, PAXBP1, CHD9, CHD7, TSPYL2, |
| | | ECD, NFATC2, ERCC3, CHD6, L3MBTL2, L3MBTL3, CREB1, SIRT7, |
| | | MRGBP, SIRT2, MED31, RPAP2, SP1, DR1, KDM4C, IRF3, TBL1X, VPS25 |
| GO: 0006351~tran- | 271 | ITGB3BP, MEF2A, BBX, RORA, ZNF638, MXI1, BRPF1, SIN3A, ZFP90, |
| sription, DNA- | | ZNF394, ZNF101, ZNF43, ZNF44, ZNF644, RXRB, PCBD1, ZHX1, MECP2, |
| templated | | MED11, HMG20B, ZNF37A, MED19, MAPK1, PIAS4, ASCC2, HES4, |
| | | MLLT10, PRDM2, CDCA7L, PIAS1, SUDS3, CRTC3, CRTC2, NFKBIB, |
| | | ZNF131, ZNF511, DIDO1, XAB2, NR1H2, MOV10, TCF20, TCF3, ASXL2, |
| | | IKZF5, ESRRA, TCF7, ZNF529, IKZF2, TP53BP1, ZNF121, KLF10, RYBP, |
| | | ZBTB40, SMAD3, RNF4, DMTF1, HOPX, JAZF1, PPRC1, KAT6B, RERE, |
| | | NCOR2, NKAP, CCNT2, CREBRF, TAF1B, ELF2, ZNF534, EZH1, CCNT1, |
| | | COPRS, ZNF675, ZEB1, RFXANK, DAXX, ZBTB38, DNAJC17, MBTD1, |
| | | ASH2L, BRD4, USP16, DEDD2, ZNF493, BRD8, SERTAD2, ATF7IP, |
| | | NFKBIZ, CTBP1, RBBP4, POLR1E, LIN52, CCNH, POLR1A, TLE3, SPEN, |
| | | MXD4, CHMP1A, ZNF277, NOL11, CNOT11, USP22, ZN1F746, ZNF740, |
| | | ZNF276, ZNF275, ZNF274, ZBTB10, ZBTB11, PML, ZNF780B, ZNF780A, |
| | | POLR2B, MYCBP2, STAT6, LPXN, RB1CC1, NPAT, GATAD2A, ZSCAN25, |
| | | BCL3, AGO2, THAP1, ACTL6A, PRKAA1, HBP1, BAZ2B, ZNF268, BAZ2A, |
| | | POLR3H, ZNF28, PPHLN1, TRIM27, PPP1R10, PHF10, POLR3C, ATMIN, |
| | | TRIM22, POLR3E, IWS1, ZNF664, ZNF672, BRMS1, JMJD6, YAF2, MAPK13, |
| | | WHSC1L1, ZBTB2, ZNF764, ZNF766, RALY, ZNF583, CNOT8, IL16, INO80, |
| | | CBX7, TCEAL4, GABPB1, DPY30, CASP8AP2, MDFIC, CGGBP1, ZNF7, |
| | | ARID1B, TRERF1, TRIM33, KDM2A, NFE2L2, AKAP8, NFE2L3, ZNF587, |
| | | TFB1M, SMARCA2, ZNF586, ZNF430, LITAF, SETD1A, KEAP1, |
| | | C14ORF166, TRRAP, COMMD9, COMMD10, RRAGC, COCCI, PELP1, |
| | | CNOT6L, RNF10, SETDB1, ZMYM2, TRIP4, CREBZF, RFX5, WDR5, |
| | | ZMYM5, NR4A1, KAT5, MED13L, FOXP3, ATF7IP2, UIMC1, SAFB2, |
| | | SREBF2, CTR9, ATF6, PHF3, PHF1, CPNE1, E2F3, E2F4, GPBP1, YLPM1, |
| | | ZKSCAN1, CBFA2T2, PCGF5, ZNF721, INO80D, KDM5B, KDM5C, INO80C, |
| | | ELMSAN1, INO80B, TBL1XR1, SSBP3, LRIF1, ELP6, LDB1, ELP5, ZFX, |
| | | ARID5A, PKN2, BANP, IRF2BP2, FOXJ3, LPIN1, NRIP1, PRKCB, NCOA1, |
| | | NCOA2, BPTF, HIPK1, BTG2, FAM120B, HIPK2, COMMD3, KHSRP, |
| | | COMMD1, WASL, JMJD1C, DPF2, ING3, SBNO2, ING2, FRYL, KMT2C, |
| | | ZNF800, NFYB, PAXBP1, CHD9, CHD7, TSPYL2, CHD6, GTF3C1, GTF3C3, |
| | | L3MBTL2, L3MBTL3, DRG1, MRGBP, SIRT2, DR1, KDM4C, TBL1X, VPS25 |
| GO: 0006355~regulation | 196 | ITGB3BP, MEF2A, BBX, MED23, ZNF638, RORA, MXI1, SIN3A, ZFP90, |
| of transcription, | | ZNF394, TBPL1, ZNF101, ZNF43, ZNF44, ZNF644, RXRB, HMG20B, |
| DNA-templated | | PPARGC1A, ZNF37A, ASCC2, HES4, CDCA7L, PRDM2, ZNHIT3, ZNF131, |
| | | ZNF511, AHCTF1, TCF20, MOV10, TCF3, ASXL2, IKZF5, ZNF529, ESRRA, |
| | | NRBF2, ZNF121, CREBBP, ZBTB40, SMAD3, PMF1, DMTF1, KAT6B, |
| | | TAF1B, CREBRF, TAF1C, ZNF534, COPRS, ZNF675, DAXX, MBTD1, |
| | | ASH2L, MYB, ZNF493, BRD8, RBBP4, BRF1, BRF2, TLE3, GTF2H3, GTF2B, |
| | | TTF2, TAF10, POGK, ZNF277, CNOT11, CDK11B, ZNF746, ZNF740, ZNF276, |
| | | ZNF275, ZNF274, ZBTB10, ZBTB11, PML, ZNF780B, ZNF780A, MLF2, |
| | | MYCBP2, ZFP36L2, LPXN, RB1CC1, ZSCAN25, PRKAA1, HBP1, THAP1, |
| | | ZNF268, BAZ2B, USP34, MLLT6, BAZ2A, ZNF28, PPHLN1, PHF10, AFF1, |
| | | TRIM22, IWS1, ZNF664, ZNF672, MAPK13, JMJD6, WHSC1L1, ZBTB2, |
| | | ZNF764, OGG1, ZNF766, GOLGB1, RALY, CNOT8, ZNF583, IL16, |
| | | AKAP17A, CASP8AP2, ZNF814, ZNF7, RALGAPA1, EP300, KDM2A, |
| | | AKAP8, TH31M, ZNF587, SMARCA2, ZNF586, ZNF430, SETD1A, KEAP1, |
| | | TRRAP, COMMD9, CXXC1, COMMD10, CNOT6L, TRIP4, RFX5, FOXP3, |
| | | KAT5, VAV1, ATF7IP2, CTR9, SAFB2, TEFM, ATF5, PHF1, CDKN2AIP, |
| | | CPNE1, HIVEP2, GPBP1, FOXK2, YLPM1, ZKSCAN1, GTF2A1, RBAK- |
| | | RBAKDN, ZNF721, ZNF720, INO80D, INO80C, INO80B, SSBP2, LRIF1, |
| | | LDB1, ZFX, ELP5, PKN2, IRF2BP2, NCOA1, NCOA2, HIPK1, BPTF, |
| | | FAM120B, COMMD3, KHSRP, JMJD1C, WASL, DPF2, SBNO1, SBNO2, |
| | | ING3, ING2, FRYL, KMT2C, ZNF800, NFYC, NFYB, GLRX2, CHD9, |
| | | TSC22D3, TSPYL2, CHD7, NFATC2, L3MBTL2, L3MBTL3, VHL, CREB1, |
| | | CBL, RGS19, SP1, POFUT1, VPS25 |
| DNA-binding | 227 | RAD51C, HMGN3, MEF2A, BBX, H1FX, ZNF638, RORA, MXI1, HMGN4, |
| | | TBPL2, BRPF1, ZFP90, ZNF394, TBPL1, ZNF101, ZNF43, POLK, ZNF44, |
| | | ZNF644, TADA2A, RXRB, ZHX1, MECP2, HMG20B, POLB, TOX4, ZNF37A, |
| | | MAPK1, UHRF2, PIAS4, MTF2, HES4, MLLT10, JUN, PRDM2, PIAS1, |
| | | ZNF131, AHCTF1, ZNF511, PIN4, NR1H2, TCF20, TCF3, PLAGL2, IKZF5, |
| | | DNMT3A, ZNF529, ESRRA, TCF7, IKZF2, KLF13, ZNF121, KLF10, TP53BP1, |
| | | RYBP, ZBTB40, SMAD3, ATM, RNF4, DMTF1, H3F3A, NCOR2, TAF1B, |
| | | TAF1C, ZNF292, ELF2, ZNF534, BACH2, ZNF675, ZEB1, RFXANK, ZBTB38, |
| | | LONP1, ASH2L, ZNF148, TOP2B, MYB, DEDD2, ZNF493, ZFP36, POGZ, |
| | | AIFM1, APTX, MBD4, PAPD5, SPEN, MBD1, TTF2, MXD4, POGK, GTF2I, |
| | | ZNF277, ZNF746, MED1, REV3L, ZNF276, ZNF275, ETV7, ZNF274, ZBTB10, |
| | | ZBTB11, PML, ZNF780B, ZNF780A, STAT6, ZFP36L2, ZSCAN25, THAP1, |
| | | HBP1, THAP2, BAZ2B, ZNF268, BAZ2A, ZNF28, TRIM27, PPP1R10, ZNF664, |
| | | PLSCR1, ZNF672, ZBTB2, ZNF764, ZNF766, ZNF583, INO80, TBP, CGGBP1, |
| | | TARDBP, GATA3, ORC4, RELA, MTA2, NEIL2, ZNF7, ARID1B, TRERF1, |
| | | NABP1, TRIM33, KDM2A, RNF138, AKAP8, NFE2L2, TH31M, ZNF587, |
| | | NFE2L3, SMARCA2, ZNF586, ZNF430, CERS6, CERS4, ELK3, SRF, CXXC1, |
| | | POLE3, CERS2, RNF10, RUNX1, RFX5, NR4A1, TSN, FOXP3, SAFB2, |
| | | SREBF2, ATF6, ATF5, NRF1, GTF2F1, GTF2F2, HIVEP2, HIVEP1, GLYR1, |
| | | IER2, HIST4H4, E2F3, E2F4, GPBP1, FOXK2, ZKSCAN1, CTCF, FOS, |
| | | ZNF721, NFX1, ELMSAN1, EGR1, SSBP3, SSBP2, ZFX, ARID5A, BANP, |
| | | FOXJ3, MCM6, NUCB1, XPA, RECQL, XPC, HIPK1, HIPK2, NUCB2, DDB2, |
| | | KHSRP, BTAF1, KMT2A, KMT2C, MGMT, ZNF800, NFYC, NFYB, PAXBP1, |
| | | APLP2, RPA1, CHD9, CHD7, HMGXB4, HMGXB3, NFATC2, ERCC3, CHD6, |
| | | GTF3C1, TERF2, GTF3C3, TERF1, MSH6, MSH2, CREB1, TOX, SP1, DR1, |
| | | IRF3 |
| Enrichment Score: | | |
| 3.4484346569016893 | | |
| Immunity | 81 | CD8A, ZC3HAV1, CD8B, PTPN22, APOBEC3G, PDCD1, APOBEC3C, |
| | | APOBEC3D, TRIM4, ANKRD17, NLRC5, GATA3, IL4R, ERAP1, JAGN1, |
| | | MX1, MX2, DBNL, IRAK1, SIT1, LY96, HERC5, FADD, ECSIT, PRKCB, |
| | | CD84, TRIM38, BTN3A1, CHID1, TNFSF13B, CAMK4, RIPK2, LRMP, HLA- |
| | | DPA1, AKAP8, GBP3, BTN3A2, ORAI1, IFITM1, IFITM2, CSF1, UNC93B1, |
| | | PML, RSAD2, OAS1, OAS2, SEC14L1, RNF125, IRAK4, SERINC3, PYCARD, |
| | | ZAP70, PSTPIP1, HLA-DPB1, INPP5D, MR1, TBKBP1, TRAF3, POLR3F, |
| | | POLR3H, MYO1G, CTLA4, ANXA1, SAMHD1, MSRB1, TRIM25, PMF1, |
| | | SLAMF7, POLR3C, LGALS9, POLR3E, SIRT2, IFIT3, BTLA, CD55, IFIT5, |
| | | CD79B, JAK2, IRF3, TAPBPL, IL2 |
| Innate immunity | 50 | ZC3HAV1, APOBEC3G, APOBEC3C, APOBEC3D, TRIM4, NLRC5, |
| | | ANKRD17, GATA3, MX1, MX2, IRAK1, LY96, HERC5, FADD, ECSIT, CD84, |
| | | TRIM38, CHID1, RIPK2, AKAP8, IFITM1, IFITM2, CSF1, PML, UNC93B1, |
| | | RSAD2, OAS1, OAS2, SEC14L1, SERINC3, IRAK4, PSTPIP1, PYCARD, MR1, |
| | | TBKBP1, POLR3F, POLR3H, ANXA1, MSRB1, SAMHD1, TRIM25, SLAMF7, |
| | | POLR3C, POLR3E, SIRT2, IFIT3, CD55, IFIT5, JAK2, IRF3 |
| GO: 0045087~innate | 56 | ZC3HAV1, APOBEC3G, IGHM, APOBEC3C, APOBEC3D, TRIM4, NLRC5, |
| immune response | | ANKRD17, GATA3, MX1, KLRD1, MX2, CHUK, MATK, IRAK1, SRPK2, |
| | | LY96, HERC5, FADD, ECSIT, SRPK1, CD84, CHID1, IPO7, RIPK2, AKAP8, |
| | | CSF1, PML, TRIM14, UNC93B1, TRDC, SEC14L1, SERINC3, IRAK4, |
| | | PSTPIP1, ZAP70, PYCARD, MR1, TBKBP1, TRAF3, POLR3F, POLR3H, |
| | | TRIM27, ANXA1, TRIM26, MSRB1, MALT1, TRIM25, POLR3C, POLR3E, |
| | | SIRT2, CD55, APOL1, IFIT5, JAK2, ABL2 |
| Enrichment Score: | | |
| 3.4148927968910225 | | |
| hsa04668: TNF | 31 | TRAF1, CSF2, TRAF2, CSF1, LIF, TNFRSF1A, BAG4, FOS, CASP7, CASP8, |
| signaling pathway | | BCL3, MAP2K7, TRAF5, CHUK, PIK3R1, AKT2, TRAF3, ICAM1, IL18R1, |
| | | SOCS3, CREB1, RELA, MAP2K4, FADD, TAB3, MAPK1, MAPK13, JUN, |
| | | RIPK1, MAPK8, MAP3K14 |
| hsa04620: Toll-like | 24 | IRAK1, CCL3, LY96, RELA, MAP2K4, FADD, CCL4, IFNAR1, IRAK4, |
| receptor signaling | | MAPK1, FOS, IFNAR2, MAPK13, JUN, RIPK1, CASP8, RAC1, IRF3, MAPK8, |
| pathway | | MAP2K7, CHUK, PIK3R1, AKT2, TRAF3 |
| hsa04380: Osteoclast | 25 | TRAF2, CSF1, FOS, TNFRSF1A, IFNG, RAC1, PPP3CB, NFATC2, MAP2K7, |
| differentiation | | IFNGR2, PIK3R1, CHUK, AKT2, SOCS3, RELA, CREB1, SOCS1, IFNAR1, |
| | | MAPK1, IFNAR2, CAMK4, MAPK13, JUN, MAPK8, MAP3K14 |
| Enrichment Score: | | |
| 3.2064615359892623 | | |
| Bromodomain | 14 | BRD1, KMT2A, CREBBP, PHIP, BRPF1, EP300, TRIM33, BPTF, ASH1L, |
| | | BRD4, BAZ2B, BAZ2A, SMARCA2, BRD8 |
| SM00297: BROMO | 14 | BRD1, KMT2A, CREBBP, PHIP, BRPF1, EP300, TRIM33, BPTF, ASH1L, |
| | | BRD4, BAZ2B, BAZ2A, SMARCA2, BRD8 |
| IPR001487: Bromodomain | 14 | BRD1, KMT2A, CREBBP, PHIP, BRPF1, EP300, TRIM33, BPTF, ASH1L, |
| | | BRD4, BAZ2B, BAZ2A, SMARCA2, BRD8 |
| IPR018359: Bromodomain, | 10 | PHIP, BRD1, BRPF1, EP300, BPTF, CREBBP, BRD4, BAZ2B, SMARCA2, |
| conserved site | | BAZ2A |
| domain: Bromo | 10 | BRD1, BRPF1, EP300, TRIM33, BPTF, CREBBP, ASH1L, BAZ2B, SMARCA2, |
| | | BAZ2A |
| Enrichment Score: | | |
| 3.1111268555872935 | | |
| mRNA processing | 63 | RALY, SCAF1, CRNKL1, ZMAT5, U2AF2, SKIV2L2, SART3, SART1, |
| | | AKAP17A, DDX23, TARDBP, CDK12, QKI, DBR1, LSM3, RBM10, LSM1, |
| | | TSEN2, CDK13, SRPK2, SYMPK, PAN3, EFTUD2, FMR1, PAPD5, CSTF2T, |
| | | PRPF4, SRPK1, TTF2, WDR83, PCF11, KHSRP, THOC6, SLU7, CPSF4, |
| | | CPSF3, FIP1L1, XAB2, SF3B4, SF3B3, PRPF19, CNOT6L, ECD, ISY1, |
| | | DHX16, GEMIN6, RBM28, GEMIN5, RBM22, TSEN54, SREK1, ALYREF, |
| | | SNW1, CASC3, SF3A2, IWS1, SUGP1, CLASRP, JMJD6, RSRC1, LSM10, |
| | | RNPC3, CSTF1 |
| mRNA splicing | 50 | RALY, SCAF1, ZMAT5, CRNKL1, U2AF2, SKIV2L2, SART3, SART1, |
| | | AKAP17A, DDX23, TARDBP, CDK12, QKI, LSM3, LSM1, RBM10, CDK13, |
| | | SRPK2, EFTUD2, FMR1, PRPF4, TTF2, SRPK1, WDR83, KHSRP, THOC6, |
| | | SLU7, XAB2, SF3B4, SF3B3, PRPF19, ECD, ISY1, DHX16, GEMIN6, RBM28, |
| | | GEMIN5, RBM22, SREK1, ALYREF, SNW1, CASC3, SF3A2, IWS1, SUGP1, |
| | | CLASRP, JMJD6, RSRC1, LSM10, RNPC3 |
| GO: 0008380~RNA | 36 | SCAF1, ZMAT5, RP9, ZNF638, IVNS1ABP, SF3B4, SF3B3, RRAGC, |
| splicing | | AKAP17A, DDX23, TARDBP, ECD, CDK12, QKI, DHX16, RBM10, LSM1, |
| | | RBM28, SRPK2, EFTUD2, SREK1, FMR1, SF3A2, PPARGC1A, PRPF4, IWS1, |
| | | TTF2, SRPK1, PPIG, CLASRP, JMJD6, RSRC1, KHSRP, THOC6, LSM10, |
| | | RNPC3 |
| GO: 0000398~mRN | 44 | RALY, FIP1L1, ZMAT5, CRNKL1, U2AF2, SKIV2L2, SART3, SF3B4, XAB2, |
| A splicing, via | | SART1, SF3B3, POLR2B, PRPF19, METTL3, DDX23, ISY1, DHX16, DBR1, |
| spliceosome | | LSM3, GEMIN6, GEMIN5, RBM22, EFTUD2, ALYREF, ELAVL1, SNW1, |
| | | CASC3, SPEN, SF3A2, PRPF4, WDR83, PCF11, HNRNPH2, UPF3B, SUGP1, |
| | | GTF2F1, RSRC1, GTF2F2, RBMX2, SLU7, RNPC3, CPSF3, RBM15, CSTF1 |
| Spliceosome | 27 | RALY, ZMAT5, CRNKL1, U2AF2, SKIV2L2, SF3B4, XAB2, SART1, SF3B3, |
| | | PRPF19, AKAP17A, DDX23, ISY1, LSM3, RBM28, RBM22, SREK1, EFTUD2, |
| | | ALYREF, SNW1, SF3A2, PRPF4, TTF2, WDR83, SUGP1, SLU7, RNPC3 |
| GO: 0006397~mRN | 37 | SCAF1, U2AF2, HNRNPLL, SF3B4, SF3B3, AKAP17A, METTL3, CNOT6L, |
| A processing | | TARDBP, ECD, CDK12, QKI, LSM3, TSEN2, RBM10, LSM1, RBM28, PHRF1, |
| | | TSEN54, RBM23, PAN3, EFTUD2, SREK1, FMR1, PAPD5, CASC3, SF3A2, |
| | | PPARGC1A, IWS1, TTF2, SRPK1, CLASRP, JMJD6, KHSRP, LSM10, CPSF4, |
| | | ALKBH5 |
| GO: 0071013~catalytic | 18 | RBM22, RALY, CRNKL1, EFTUD2, ALYREF, SKIV2L2, SNW1, SF3A2, |
| step 2 | | XAB2, SART1, SF3B3, WDR83, PRPF19, DDX23, ISY1, RBMX2, SLU7, |
| spliceosome | | LSM3 |
| hsa03040: Spliceosome | 22 | RBM22, CHERP, CRNKL1, CCDC12, U2AF2, EFTUD2, ALYREF, SNW1, |
| | | HSPA1A, SF3A2, PRPF4, XAB2, SF3B4, SART1, SF3B3, PRPF19, DDX23, |
| | | ISY1, SLU7, DHX16, LSM3, DDX42 |
| Enrichment Score: | | |
| 3.0807772438702044 | | |
| hsa03022: Basal | 16 | TAF6, CCNH, TAF9B, GTF2H3, TBP, CDK7, GTF2B, TBPL2, TAF10, TAF13, |
| transcription factors | | GTF2A1, GTF2I, GTF2F1, GTF2F2, ERCC3, TBPL1 |
| GO: 0006367~transcription | 33 | E2F3, TAF9B, MED23, TBP, RORA, POLR2B, NR1H2, GTF2A1, ERCC3, |
| initiation | | ESRRA, NRBF2, TAF6, CCNH, RXRB, CREBBP, GTF2H3, CDK9, NR4A1, |
| from RNA | | SNW1, MED13, CDK7, GTF2B, PPARGC1A, MED31, TAF10, TAF13, GTF2I, |
| polymemse II | | MED16, GTF2F1, MED8, MED17, GTF2F2, MEDI |
| promoter | | |
| GO: 0006368~transcription | 19 | CCNT2, ELP2, TAF6, CCNH, CCNT1, TAF9B, GTF2H3, CDK9, TBP, CDK7, |
| elongation | | GTF2B, POLR2B, TAF10, TAF13, GTF2A1, GTF2F1, GTF2F2, LEO1, ERCC3 |
| from RNA | | |
| polymerase II | | |
| promoter | | |
| Enrichment Score: | | |
| 3.020215334274786 | | |
| hsa04662: B cell | 22 | VAV3, NFKBIB, RELA, RAF1, MALT1, VAV1, NRAS, MAPK1, FOS, KRAS, |
| receptor signaling | | JUN, GSK3B, SOS1, CD81, RAC1, PPP3CB, CD79B, INPP5D, NFATC2, |
| pathway | | CHUK, PIK3R1, AKT2 |
| h_fcer1Pathway: Fc | 14 | MAP2K4, RAF1, VAV1, PRKCB, MAPK1, FOS, MAP3K1, SOS1, JUN, |
| Epsilon Receptor I | | PPP3CB, MAPK8, NFATC2, MAP2K7, PIK3R1 |
| Signaling in Mast | | |
| Cells | | |
| GO: 0038095~Fc- | 30 | PSMB10, FOS, KRAS, MAP3K1, SOS1, RAC1, PPP3CB, PSMD3, PSMD5, |
| epsilon receptor | | NFATC2, MAP2K7, FBXW11, CHUK, PIK3R1, VAV3, RELA, MAP2K4, |
| signaling pathway | | MALT1, VAV1, TAB3, NRAS, MAPK1, PSMC5, PSMD13, PSMD12, JUN, |
| | | PSMC2, MAPK8, PSME4, GRAP2 |
| Enrichment Score: | | |
| 2.697299540563712 | | |
| hsa04722: Neurotrophin | 30 | ZNF274, NFKBIB, MAPKAPK2, IRAK4, KRAS, MAP3K3, BCL2, SOS1, |
| signaling | | MAP3K1, RAC1, MAP2K7, RAPGEF1, PIK3R1, MATK, AKT2, IRAK1, |
| pathway | | RELA, RAF1, BAD, PRKCD, NRAS, MAPK1, RPS6KA3, CRKL, CAMK4, |
| | | MAPK13, JUN, GSK3B, RIPK2, MAPK8 |
| hsa04012: EibB | 21 | MAP2K4, CBL, RAF1, BAD, PRKCB, NRAS, MAPK1, NCK2, CBLB, CRKL, |
| signaling pathway | | KRAS, CDKN1B, JUN, GSK3B, SOS1, ARAF, MAPK8, MAP2K7, ABL2, |
| | | PIK3R1, AKT2 |
| hsa04912: GnRH | 17 | ADCY7, MAP2K4, RAF1, PRKCD, PRKCB, ITPR2, NRAS, MAPK1, KRAS, |
| signaling pathway | | MAP3K3, MAPK13, JUN, MAP3K1, SOS1, MAPK8, PRKACB, MAP2K7 |
| Enrichment Score: | | |
| 2.6555136612397368 | | |
| domain.LisH | 10 | OFD1, TBL1XR1, SMU1, MKLN1, SSBP3, SSBP2, WDR26, NPAT, |
| | | PAFAH1B1, TBL1X |
| SM00667: LisH | 9 | OFD1, TBL1XR1, SMU1, MKLN1, SSBP3, SSBP2, NPAT, PAFAH1B1, |
| | | TBL1X |
| IPR006594: LisH | 10 | OFD1, TBL1XR1, SMU1, MKLN1, SSBP3, SSBP2, WDR26, NPAT, |
| dimerisation motif | | PAFAH1B1, TBL1X |
| Enrichment Score: | | |
| 2.5486858895909723 | | |
| Nuclear pore | 17 | NUP98, NUP160, AHCTF1, NUP93, NUP85, NUP188, PARP11, NUP155, |
| complex | | NDC1, NUP214, DDX19A, NUP210, RANBP2, XPO7, MX2, EIF5A2, MVP |
| GO: 0016925~protein | 30 | NUP98, NUP160, PML, NUP93, CETN2, SAE1, NUP188, RANGAP1, NDC1, |
| sumoylation | | RPA1, NUP214, MDC1, NUP210, NSMCE1, NSMCE2, RNF168, RANBP2, |
| | | TOP2B, STAG1, L3MBTL2, KIAA1586, TP53BP1, SMC5, SMC6, NUP85, |
| | | HERC2, NUP155, XPC, PIAS4, PIAS1 |
| GO: 1900034~regulation | 22 | NUP98, NUP160, CREBBP, NUP93, NUP85, NUP188, HSPA1A, MAPKAPK2, |
| of cellular | | NUP155, ATM, NDC1, RPA1, BAGS, BAG4, MAPK1, NUP214, EP300, |
| response to heat | | GSK3B, NUP210, MLST8, RANBP2, DNAJB6 |
| mRNA transport | 26 | NUP98, NUP160, NUP93, AHCTF1, NUP188, NDC1, NUP214, DDX19A, |
| | | NUP210, QKI, RANBP2, MX2, FMR1, ALYREF, NUP85, PARP11, CASC3, |
| | | NUP155, IWS1, UPF3B, POLDIP3, THOC6, KHSRP, XPO7, EIF5A2, MVP |
| Translocation | 21 | NUP98, NUP160, AHCTF1, NUP93, NUP85, NUP188, PARP11, CHCHD4, |
| | | NUP155, TIMM22, TIMM8A, NDC1, NUP214, DNAJC15, DDX19A, NUP210, |
| | | RANBP2, XPO7, MX2, EIF5A2, MVP |
| GO: 0005643~nuclear | 19 | NUP98, NUP160, AHCTF1, NUP93, PARP11, RANGAP1, NUP155, NDC1, |
| pore | | IPO7, DDX19A, NUP210, KPNA6, NUTF2, RANBP2, XPO7, MX2, EIF5A2, |
| | | KPNA1, MVP |
| GO: 0006406~mRN | 24 | NUP98, FIP1L1, NUP160, SMG5, U2AF2, ALYREF, NUP93, NUP85, NUP188, |
| A export from | | CASC3, NUP155, NDC1, NUP214, UPF3B, EIF4E, DDX19A, POLDIP3, |
| nucleus | | NUP210, RBMX2, THOC6, SLU7, RANBP2, CPSF3, ALKBH5 |
| GO: 0007077~mitotic | 13 | NDC1, NUP214, NUP98, NUP160, NUP210, NUP93, CNEP1R1, NUP85, |
| nuclear envelope | | NUP188, RANBP2, NUP155, LPIN1, PRKCB |
| disassembly | | |
| hsa03013: RNA | 32 | RPP38, NUP98, ELAC2, NUP160, NUP93, RANGAP1, NUP188, NDC1, |
| transport | | NUP214, EIF4EBP2, NUP210, RANBP2, GEMIN6, EIF2B4, EIF2B5, GEMIN5, |
| | | ALYREF, FMR1, NUP85, EIF1B, CASC3, FXR2, NUP155, TACC3, EIF2B1, |
| | | EIF4G3, EIF4E, UPF3B, THOC6, POP4, POPS, POP7 |
| GO: 0006409~tRNA | 10 | NDC1, NUP214, NUP98, NUP160, NUP210, NUP93, NUP85, NUP188, |
| export from nucleus | | RANBP2, NUP155 |
| GO: 0010827~regulation | 10 | NDC1, NUP214, NUP98, NUP160, NUP210, NUP93, NUP85, NUP188, |
| of glucose transport | | RANBP2, NUP155 |
| GO: 0075733~intracellular | 13 | NDC1, NUP214, NUP98, NUP160, TSG101, NUP210, NUP93, VPS37B, |
| transport of virus | | NUP85, NUP188, RANBP2, NUP155, KPNA1 |
| GO: 0017056~structural | 7 | NDC1, NUP214, NUP98, NUP93, NUP85, NUP188, NUP155 |
| constituent of nuclear pore | | |
| GO: 0031047~gene | 21 | NUP98, HIST4H4, NUP160, FMR1, DICER1, NUP93, NUP85, NUP188, TSN, |
| silencing by RNA | | NUP155, POLR2B, NDC1, NUP214, CNOT6L, NUP210, PRKRA, CNOT11, |
| | | H3F3A, AGO2, RANBP2, TNRC6A |
| GO: 0006606~protein | 11 | NUP214, IPO7, PTTG1IP, NUP93, KPNA6, PPP1R10, NUP85, NUTF2, |
| import into nucleus | | NUP188, NUP155, KPNA1 |
| GO: 0019083~vira1 | 11 | NDC1, RPL17, NUP214, NUP98, NUP160, NUP210, NUP93, NUP85, NUP188, |
| transcription | | RANBP2, NUP155 |
| Enrichment Score: | | |
| 2.3663661637495768 | | |
| IPR009060: UBA- | 18 | USP5, CBL, UBAC1, LATS1, N4BP2, CBLB, C6ORF106, ASCC2, TDP2, |
| like | | NBR1, TSFM, UBAP2L, FAF2, UBASH3A, SPATS2L, UBAP2, USP24, UBAP1 |
| IPR015940: Ubiquitin- | 13 | USP5, CBL, UBAC1, LATS1, MARK2, CBLB, SNRK, NBR1, UBAP2L, |
| associated/translation | | UBASH3A, UBAP2, USP24, UBAP1 |
| elongation factor | | |
| EF1B, N-terminal, | | |
| eukaryote | | |
| domain: UBA | 11 | CBLB, SNRK, NBR1, CBL, UBAP2L, UBASH3A, FAF2, UBAP2, USP24, |
| | | LATS1, MARK2 |
| SM00165: UBA | 7 | CBLB, USP5, CBL, UBAP2L, UBAC1, UBAP2, MARK2 |
| Enrichment Score: | | |
| 2.185234142205886 | | |
| GO: 1904115~axon | 13 | KIF3B, SPG7, NDEL1, BLOC1S5, AP3M2, AP3M1, BLOC1S1, AP3S1, |
| cytoplasm | | SNAPIN, RANGAP1, PAFAH1B1, DTNBP1, SPAST |
| GO: 0008089~antero | 10 | SPG7, KIF3B, BLOC1S5, AP3M2, AP3M1, BLOC1S1, AP3S1, SNAPIN, |
| grade axonal transport | | DTNBP1, SPAST |
| GO: 0048490~antero | 7 | BLOC1S5, AP3M2, AP3M1, BLOC1S1, AP3S1, SNAPIN, DTNBP1 |
| grade synaptic | | |
| vesicle transport | | |
| GO: 0031083~BLOC-1 | 6 | BLOC1S5, KXD1, BLOC1S1, KIAA1033, SNAPIN, DTNBP1 |
| complex | | |
| GO: 0032438~melanosome | 5 | BLOC1S5, AP1G1, BLOC1S1, SNAPIN, DTNBP1 |
| organization | | |
| Enrichment Score: | | |
| 2.109254171296502 | | |
| IPR004939: Anaphase- | 6 | HSPB11, CUL9, ANAPC10, HERC2, ZZEF1, MYCBP2 |
| promoting complex, subunit | | |
| 10/DOC domain | | |
| SM01337: SM01337 | 5 | CUL9, ANAPC10, HERC2, ZZEF1, MYCBP2 |
| domain: DOC | 5 | CUL9, ANAPC10, HERC2, ZZEF1, MYCBP2 |
| IPR008979: Galactose- | 13 | ANAPC10, HERC2, FURIN, ZZEF1, MYCBP2, NGLY1, MKLN1, HSPB11, |
| binding domain-like | | CUL9, FBXO6, PCSK7, HECTD1, SUCO |
| Enrichment Score: | | |
| 2.052960731204601 | | |
| GO: 0005913~cell- | 54 | RTN4, ABCF3, LIMA1, ZC3HAV1, VAPB, H1FX, RANGAP1, EFHD2, SLK, |
| cell adherens | | LRRC59, ZYX, DBNL, BSG, PKN2, TXNDC9, CLIC1, FLNA, MARK2, CRKL, |
| junction | | DHX29, USO1, SDCBP, MAPRE1, UBAP2, CD226, ADD1, SNX2, ASAP1, |
| | | KEAP1, HSPA1A, ITGB1, SH3GLB2, RAB11B, FASN, CNN2, CCS, EHD1, |
| | | EHD4, PLEC, APC, CBL, ARFIP2, S100A11, ANXA1, TRIM25, GLOD4, |
| | | TMEM2, ANXA2, TIGIT, CSNK1D, LASP1, AHSA1, YKT6, CD200 |
| GO: 0098641~cadherin | 48 | RTN4, ABCF3, LIMA1, ZC3HAV1, VAPB, SNX2, ASAP1, H1FX, HSPA1A, |
| binding involved | | RANGAP1, ITGB1, EFHD2, SLK, SH3GLB2, LRRC59, FASN, RAB11B, |
| in cell-cell adhesion | | CNN2, CCS, EHD1, PLEC, EHD4, DBNL, BSG, CBL, ANXA1, S100A11, |
| | | ARFIP2, PKN2, TRIM25, TXNDC9, CLIC1, GLOD4, FLNA, ANXA2, MARK2, |
| | | TMEM2, CRKL, CSNK1D, DHX29, LASP1, USO1, SDCBP, MAPRE1, UBAP2, |
| | | YKT6, AHSA1, ADD1 |
| GO: 0098609~cell- | 45 | RTN4, LIMA1, ABCF3, ZC3HAV1, VAPB, SNX2, ASAP1, H1FX, HSPA1A, |
| cell adhesion | | RANGAP1, EFHD2, SLK, SH3GLB2, LRRC59, FASN, RAB11B, CNN2, CCS, |
| | | EHD1, PLEC, EHD4, DBNL, BSG, CREBBP, CBL, S100A11, ARFIP2, PKN2, |
| | | TRIM25, TXNDC9, GLOD4, ANXA2, MARK2, TMEM2, CRKL, CSNK1D, |
| | | DHX29, LASP1, USO1, SDCBP, MAPRE1, UBAP2, YKT6, AHSA1, ADD1 |
| Enrichment Score: | | |
| 1.9864300666381447 | | |
| IPRO05225: Small | 34 | RAB5B, RAB5C, ARF6, MTIF2, GFM2, ARL5A, KRAS, GFM1, RAC1, RALB, |
| GTP-binding protein | | RAB11B, SAR1B, RHOF, ARL2, RAP2C, EFTUD2, DRG1, DRG2, RAB33A, |
| domain | | RAB33B, ARL3, NRAS, RAB30, RAB18, RAB35, ARF4, RHOT1, RAB5A, |
| | | RHOT2, RIT1, ARL8B, ARL4C, NKIRAS2, ARL4A |
| GO: 0007264~small | 47 | RAB5B, RAB5C, RGL4, IQGAP2, ARF6, RRAGC, DOCK2, ARL5A, KRAS, |
| GTPase mediated | | SOS1, RAC1, RAPGEF6, RAB11B, DOCK10, RAPGEF1, RHOF, ARL2, |
| signal transduction | | RAP2C, VAV3, RABIF, RALBP1, ARFIP2, RGS19, CHP1, DOCK8, VAV1, |
| | | RAB33A, RALGDS, RAB33B, ARL3, ARHGAP30, NRAS, SH2D3C, RAB30, |
| | | SH2D3A, RAB18, RAB35, KRIT1, ARF4, RAB5A, RHOT1, RHOT2, RIT1, |
| | | ARL8B, ARL4C, NKIRAS2, ARL4A |
| GO: 0003924~GTPa | 41 | GNA13, RAB5B, RAB5C, HBS1L, GTPBP10, ATL3, GNL3L, ARF6, MTIF2, |
| se activity | | RRAGC, GFM2, KRAS, GFM1, RAC1, RAB11B, RALB, TUBA1A, MX1, |
| | | SAR1B, RHOF, MX2, TUBA1C, ARL2, DNM3, NUDT1, EFTUD2, RAB33A, |
| | | RAB33B, ARL3, RAB30, RAB18, RAB35, ARF4, RAB5A, RHOT1, RHOT2, |
| | | RIT1, ARL8B, ARL4C, GBP3, NKIRAS2 |
| nucleotide | 48 | GNA13, GPN3, RAB5B, RAB5C, HBS1L, GTPBP10, ATL3, GNL3L, ARF6, |
| phosphate-binding | | MTIF2, RRAGC, GFM2, ARL5A, KRAS, GFM1, RAC1, RAB11B, RALB, |
| region: GTP | | TUBA1A, MX1, SAR1B, RHOF, MX2, TUBA1C, ARL2, DNM3, RAP2C, |
| | | GIMAP5, NIN, EFTUD2, DRG1, DRG2, PCK2, RAB33A, RAB33B, ARL3, |
| | | NRAS, RAB30, RAB18, RAB35, ARF4, RAB5A, RIT1, ARL8B, ARL4C, GBP3, |
| | | NKIRAS2, ARL4A |
| GTP-binding | 51 | GNA13, RAB5B, RAB5C, GTPBP10, ATL3, HBS1L, GNL3L, ARL5A, RALB, |
| | | SAR1B, MX1, TUBA1A, RHOF, MX2, TUBA1C, ARL2, RAP2C, EFTUD2, |
| | | ARL3, RAB18, RAB5A, ARL8B, GBP3, ARL4C, NKIRAS2, ARL4A, GPN3, |
| | | ARF6, MTIF2, RRAGC, GFM2, KRAS, GFM1, RAC1, RAB11B, DNM3, |
| | | GIMAP5, NIN, DRG1, DRG2, PCK2, RAB33A, RAB33B, GIMAP1, NRAS, |
| | | RAB30, RAB35, ARF4, RHOT1, RHOT2, RIT1 |
| IPR027417: P-loop | 119 | GNA13, RAD51C, DYNC1LI2, HBS1L, IQGAP2, INO80, NLRC5, DDX23, |
| containing | | DHX34, RALB, AAGAB, ORC4, VPS4A, DDX10, MX1, SAR1B, MX2, |
| nucleoside | | EFTUD2, IFI44, DHX29, RAB18, RFC2, ARL8B, GBP3, SMARCA2, GPN3, |
| triphosphate | | PFKFB3, MYO9B, ARF6, MTIF2, RRAGC, GFM2, MOV10, KRAS, GFM1, |
| hydrolase | | RAC1, DDX42, SMG9, KIF3B, MYO1G, ABCB7, RAB33A, RAB33B, PSMC5, |
| | | PSMC2, ARF4, RHOT1, DDX50, RHOT2, RIT1, DDX51, ABCF3, SPG7, |
| | | RAB5B, RAB5C, ATL3, GTPBP10, DICER1, YLPM1, GNL3L, HELZ, |
| | | SKIV2L2, SLFN5, PMVK, ARL5A, ATAD3A, LONP1, DYNC1H1, RHOF, |
| | | ARL2, RAP2C, MPP6, DGUOK, NDUFA10, TTF2, RAD50, MCM6, ARL3, |
| | | TOR2A, CBWD2, RECQL, RAB5A, ARL4C, NKIRAS2, SPAST, ARL4A, |
| | | BTAF1, SBNO1, SBNO2, WRNIP1, DCK, N4BP2, CHD9, CHD1L, CHD7, |
| | | KTI12, DDX19A, RAB11B, DHX16, UCK1, CHD6, EHD1, ERCC3, SPATA5, |
| | | EHD4, DNM3, MSH6, GIMAP5, MSH2, SMC5, SMC6, DRG1, DRG2, |
| | | GIMAP1, SMC4, NRAS, RAB30, RAB35, SAMD9 |
| GO: 0005525~GTP | 58 | GNA13, RAB5B, RAB5C, HBS1L, GTPBP10, ATL3, GNL3L, ARL5A, RALB, |
| binding | | TUBA1A, SAR1B, MX1, RHOF, MX2, TUBA1C, ARL2, RAP2C, EFTUD2, |
| | | ARL3, RAB18, RAB5A, ARL8B, IRGQ, GBP3, ARL4C, NKIRAS2, ARL4A, |
| | | GPN3, GLUD2, ARF6, MTIF2, RRAGC, GFM2, KRAS, GFM1, RAC1, |
| | | RAB11B, ERCC3, EHD1, EHD4, DNM3, GIMAP5, NIN, ARFIP2, DRG1, |
| | | DRG2, PCK2, RAB33A, GIMAP1, RAB33B, NRAS, RAB30, RAB35, ARF4, |
| | | RHOT1, RHOT2, RIT1, C9ORF69 |
| IPR001806: Small | 19 | RAP2C, RAB5B, RAB5C, RAB33A, RAB33B, NRAS, RAB30, KRAS, RAB18, |
| GTPase superfamily | | RAB35, RAC1, RALB, RAB11B, RHOT1, RAB5A, RHOT2, RIT1, RHOF, |
| | | NKIRAS2 |
| Enrichment Score: | | |
| 1.9444899254576018 | | |
| GO: 0097296~activation | 7 | TNFRSF10A, TRAF2, RIPK1, CASP8, SMAD3, FADD, JAK2 |
| of cysteine-type | | |
| endopeptidase | | |
| activity involved in | | |
| apoptotic signaling | | |
| pathway | | |
| GO: 0097191~extrinsic | 11 | TNFRSF10A, HIPK1, RIPK1, CASP8, IFNG, PML, SMAD3, FADD, JAK2, |
| apoptotic signaling pathway | | BAD, CD27 |
| GO: 0006919-activat | 17 | TRAF2, AIFM1, PML, SMAD3, FADD, BAD, BCL2L11, TNFRSF10A, |
| ion of cysteine-type | | SLC11A2, CDKN1B, CASP8AP2, BBC3, RIPK1, CASP8, PYCARD, JAK2, |
| endopeptidase | | DAP |
| activity involved in | | |
| apoptotic process | | |
| Enrichment Score: | | |
| 1.9286031923588833 | | |
| SM00291: ZnF_ZZ | 7 | EP300, NBR1, MIB2, UTRN, CREBBP, HERC2, ZZEF1 |
| IPR000433: Zinc | 7 | EP300, NBR1, MIB2, UTRN, CREBBP, HERC2, ZZEF1 |
| finger, ZZ-type | | |
| zinc finger | 6 | EP300, NBR1, MIB2, UTRN, CREBBP, HERC2 |
| region: ZZ-type | | |
| Enrichment Score: | | |
| 1.9043308100071867 | | |
| IPR000571: Zinc | 17 | ZFP36, RBM22, MKRN1, PAN3, ZC3H7A, ZMAT5, ZC3HAV1, ZC3H18, |
| finger, CCCH-type | | ZC3H7B, PPP1R10, HELZ, ZFP36L2, PARP12, CPSF4, ZC3H12D, RNF113A, |
| | | DUS3L |
| SM00356: ZnF_C3H1 | 14 | ZFP36, MKRN1, RBM22, PAN3, ZMAT5, ZC3H18, ZC3H7A, ZC3H7B, |
| | | PPP1R10, HELZ, ZFP36L2, PARP12, CPSF4, RNF113A |
| zinc finger | 9 | ZFP36, MKRN1, ZFP36L2, ZC3H7A, ZC3HAV1, PARP12, ZC3H7B, CPSF4, |
| region: C3H1-type 2 | | DUS3L |
| zinc finger | 9 | ZFP36, MKRN1, ZFP36L2, ZC3H7A, ZC3HAV1, PARP12, ZC3H7B, CPSF4, |
| region: C3H1-type 1 | | DUS3L |
| zinc finger | 7 | RBM22, ZC3H18, ZMAT5, PPP1R10, HELZ, ZC3H12D, RNF113A |
| region: C3H1-type | | |
| zinc finger | 5 | MKRN1, ZC3HAV1, PARP12, ZC3H7B, CPSF4 |
| region: C3H1-type 4 | | |
| zinc finger | 6 | MKRN1, ZC3H7A, ZC3HAV1, PARP12, ZC3H7B, CPSF4 |
| region: C3H1-type 3 | | |
| Enrichment Score: | | |
| 1.889023537159269 | | |
| active site: Glycyl | 18 | UBE2A, UBE2Z, UBE2G1, HERC6, UBE2J1, HERC5, BIRC6, UBA5, HERC2, |
| thioester | | UBE2J2, UBE2R2, UBE2D4, UBA3, UBE2W, SMURF2, HECTD4, HECTD1, |
| intermediate | | UBE2E1 |
| IPR016135: Ubiquitin- | 14 | UBE2A, UBE2Z, TSG101, UBE2G1, IMPACT, UBE2J1, BIRC6, UBE2J2, |
| conjugating enzyme/RWD-like | | UBE2R2, UBE2D4, KRAS, UBE2W, RWDD3, UBE2E1 |
| GO: 0061631-ubiquitin | 9 | UBE2D4, UBE2A, UBE2Z, UBE2G1, UBE2J1, BIRC6, UBE2J2, UBE2E1, |
| conjugating enzyme activity | | UBE2R2 |
| IPR000608: Ubiquitin- | 11 | UBE2D4, UBE2A, UBE2Z, KRAS, UBE2G1, UBE2J1, UBE2W, BIRC6, |
| conjugating enzyme, E2 | | UBE2J2, UBE2E1, UBE2R2 |
| IPR023313: Ubiquitin- | 6 | UBE2D4, UBE2A, KRAS, UBE2G1, UBE2E1, UBE2R2 |
| conjugating enzyme, active site | | |
| Enrichment Score: | | |
| 1.888346683338545 | | |
| IPR000313: PWWP | 8 | BRD1, DNMT3A, MSH6, BRPF1, PWWP2A, WHSC1L1, MBD5, GLYR1 |
| domain: PWWP | 7 | BRD1, DNMT3A, MSH6, BRPF1, PWWP2A, MBD5, GLYR1 |
| SM00293: PWWP | 7 | BRD1, DNMT3A, MSH6, BRPF1, PWWP2A, WHSC1L1, GLYR1 |
| Enrichment Score: | | |
| 1.8759534254878043 | | |
| short sequence | 9 | ASXL2, CHD9, NCOA1, NCOA2, PELP1, MED13, MED13L, NRIP1, MED1 |
| motif: LXXLL motif 1 | | |
| short sequence | 9 | ASXL2, CHD9, NCOA1, NCOA2, PELP1, MED13, MED13L, NRIP1, MED1 |
| motif: LXXLL motif 2 | | |
| short sequence | 5 | CHD9, NCOA1, NCOA2, PELP1, NRIP1 |
| motif: LXXLL motif 4 | | |
| short sequence | 5 | CHD9, NCOA1, NCOA2, PELP1, NRIP1 |
| motif: LXXLL motif 3 | | |
| short sequence | 4 | CHD9, NCOA1, PELP1, NRIP1 |
| motif: LXXLL motif 5 | | |
| short sequence | 3 | NCOA1, PELP1, NRIP1 |
| motif: LXXLL motif 7 | | |
| GO: 0035257~nuclear | 7 | NCOA1, NCOA2, EP300, ACTN4, SNW1, NRIP1, MED1 |
| hormone receptor binding | | |
| short sequence | 3 | NCOA1, PELP1, NRIP1 |
| motif: LXXLL motif 6 | | |
| Enrichment Score: | | |
| 1.8475951130819195 | | |
| GO: 0036258~multivesicular | 14 | CHMP3, TSG101, VTA1, CHMP6, CHMP4A, STAM2, CHMP7, VPS37B, |
| body assembly | | CHMP1A, VPS4A, HGS, STAM, PDCD6IP, VPS25 |
| GO: 0016197~endosomal | 19 | CHMP3, TSG101, STAM2, CHMP6, VTA1, CHMP4A, CHMP7, KIAA0196, |
| transport | | VPS37B, WAS, DPY30, RAB35, BLOC1S1, KIAA1033, VPS4A, HGS, STAM, |
| | | AP5M1, VPS25 |
| GO: 0006997~nucleus | 10 | NUMA1, CHMP1A, CHMP3, CHMP4A, CHMP6, CHMP7, VPS4A, H3F3A, |
| organization | | PDCD6IP, BIN1 |
| GO: 0039702~viral | 9 | CHMP1A, CHMP3, TSG101, CHMP4A, CHMP6, CHMP7, VPS4A, VPS37B, |
| budding via host ESCRT complex | | PDCD6IP |
| GO: 0000815~ESCR | 6 | CHMP1A, CHMP3, CHMP4A, CHMP6, CHMP7, VPS4A |
| T III complex | | |
| GO: 0019058~viral | 10 | CHMP3, TSG101, CHMP4A, CHMP6, VTA1, CHMP7, VPS4A, VPS37B, |
| life cycle | | PDCD6IP, FURIN |
| GO: 0000920~cell | 7 | CHMP1A, CHMP3, CHMP4A, CHMP6, CHMP7, VPS4A, PDCD6IP |
| separation after cytokinesis | | |
| GO: 0007080~mitotic | 10 | CUL3, CHMP1A, CHMP3, CHMP4A, CHMP6, CHMP7, VPS4A, CDC23, |
| metaphase plate congression | | PIBF1, PDCD6IP |
| GO: 1903774~positive regulation | 3 | TSG101, VPS4A, VPS37B |
| of viral budding via host | | |
| ESCRT complex | | |
| IPR005024: Snf7 | 5 | CHMP1A, CHMP3, CHMP4A, CHMP6, CHMP7 |
| GO: 0007034~vacuolar transport | 5 | CHMP1A, CHMP4A, CHMP6, CHMP7, ATP6V0D1 |
| GO: 1904903~ESCR | 4 | CHMP1A, VTA1, CHMP7, VPS4A |
| T III complex disassembly | | |
| GO: 1902188~positive regulation | 4 | CHMP3, TSG101, VPS4A, VPS37B |
| of viral release from host cell | | |
| GO: 0010824~regulation of | 4 | CHMP1A, CHMP3, FBXW5, PDCD6IP |
| centrosome duplication | | |
| GO: 1901673~regulation of mitotic | 3 | CHMP1A, CHMP3, PDCD6IP |
| spindle assembly | | |
| Enrichment Score: | | |
| 1.827917602900078 | | |
| GO: 0003684~damaged | 19 | MSH6, POLK, MSH2, CREBBP, NEIL2, MGMT, APTX, GTF2H3, POLB, |
| DNA binding | | RAD1, RPAL XPA, MPG, XPC, EP300, DDB2, CUL4B, OGG1, ERCC3 |
| GO: 0006294~nucleotide-excision | 11 | RPA1, XPA, XPC, CHD1L, CCNH, DDB2, GTF2H3, CETN2, CDK7, CUL4B, |
| repair, preincision complex | | ERCC3 |
| assembly | | |
| GO: 0000717~nucleotide-excision | 8 | XPA, XPC, CHD1L, DDB2, GTF2H3, CETN2, CUL4B, ERCC3 |
| repair, DNA duplex unwinding | | |
| GO: 0006283~transcription-coupled | 17 | POLK, CCNH, COPS7A, GTF2H3, COPS7B, CDK7, COPS8, XAB2, POLR2B, |
| nucleotide-excision repair | | PRPF19, RPA1, XPA, EP300, RFC2, ISY1, CUL4B, ERCC3 |
| GO: 0000715~nucleotide-excision | 8 | XPA, XPC, DDB2, COPS7A, CETN2, COPS7B, COPS8, CUL4B |
| repair, DNA damage recognition | | |
| hsa03420: Nucleotid e | 12 | RPA1, XPA, XPC, CCNH, POLE3, RFC2, DDB2, GTF2H3, CETN2, CDK7, |
| excision repair | | CUL4B, ERCC3 |
| GO: 0070911~global genome | 9 | XPA, XPC, CHD1L, DDB2, GTF2H3, CETN2, CUL4B, ERCC3, RNF111 |
| nucleotide-excision repair | | |
| GO: 0033683~nucleotide-excision | 10 | RPA1, XPA, POLK, CHD1L, RFC2, DDB2, GTF2H3, CUL4B, OGG1, ERCC3 |
| repair, DNA incision | | |
| GO: 0006293~nucleotide-excision | 7 | RPA1, XPA, CHD1L, DDB2, GTF2H3, CUL4B, ERCC3 |
| repair, preincision complex | | |
| stabilization | | |
| GO: 0070914~UV- | 5 | XPA, XPC, DDB2, INO80, CUL4B |
| damage excision repair | | |
| GO: 0006295~nucleotide-excision | 7 | RPA1, XPA, CHD1L, DDB2, GTF2H3, CUL4B, ERCC3 |
| repair, DNA incision, 3′-to lesion | | |
| GO: 0006289~nucleotide- | 10 | RPA1, XPA, XPC, NEIL2, HUS1, DDB2, GTF2H3, CETN2, OGG1, ERCC3 |
| excision repair | | |
| GO: 0006296-nucleotide-excision | 9 | RPA1, XPA, POLK, CHD1L, RFC2, DDB2, GTF2H3, CUL4B, ERCC3 |
| repair, DNA incision, 5′-to lesion | | |
| Xeroderma | 4 | XPA, XPC, DDB2, ERCC3 |
| pigmentosum | | |
| Enrichment Score: | | |
| 1.8167726915130153 | | |
| SM00154: ZnF_AN1 | 5 | ZFAND6, ZFAND5, ZFAND2A, ZFAND2B, ZFAND1 |
| IPR000058: Zinc | 5 | ZFAND6, ZFAND5, ZFAND2A, ZFAND2B, ZFAND1 |
| finger, AN1-type | | |
| zinc finger | 3 | ZFAND2A, ZFAND2B, ZFAND1 |
| region: AN1-type 2 | | |
| zinc finger | 3 | ZFAND2A, ZFAND2B, ZFAND1 |
| region: AN1-type 1 | | |
| Enrichment Score: | | |
| 1.8153950109695944 | | |
| Centromere | 27 | ITGB3BP, ZNF276, PPP2R5A, KNTC1, AHCTF1, CTCF, RANGAP1, DAXX, |
| | | ZNF330, NDE1, PPP2CB, STAG1, CSNK1A1, BOD1, CENPM, KANSL1, |
| | | TP53BP1, FMR1, DYNLT3, NUP85, DCTN5, PMF1, DCTN6, NDEL1, RCC2, |
| | | NSL1, NGDN |
| Kinetochore | 18 | CSNK1A1, ZNF276, ITGB3BP, BOD1, CENPM, KANSL1, TP53BP1, KNTC1, |
| | | DYNLT3, AHCTF1, NUP85, DCTN5, RANGAP1, PMF1, DCTN6, NDE1, |
| | | NDEL1, NSL1 |
| GO: 0000777~condensed | 16 | CSNK1A1, ITGB3BP, ZNF276, BOD1, CENPM, KANSL1, TP53BP1, KNTC1, |
| chromosome kinetochore | | DYNLT3, AHCTF1, NUP85, DCTN5, RANGAP1, DCTN6, NDE1, NDEL1 |
| Enrichment Score: | | |
| 1.7131033299195293 | | |
| hsa05210: Colorectal | 19 | MSH6, TCF7, MSH2, RAF1, SMAD3, BAD, RALGDS, MAPK1, FOS, KRAS, |
| cancer | | JUN, BCL2, GSK3B, ARAF, RAC1, MAPK8, PIK3R1, AKT2, APC |
| hsa05211: Renal cell | 18 | VHL, CREBBP, RAF1, EGLN1, CUL2, NRAS, MAPK1, EP300, CRKL, KRAS, |
| carcinoma | | JUN, SOS1, RAC1, ARAF, RAPGEF1, PIK3R1, AKT2, FH |
| hsa04012: ErbB | 21 | MAP2K4, CBL, RAF1, BAD, PRKCB, NRAS, MAPK1, NCK2, CBLB, CRKL, |
| signaling pathway | | KRAS, CDKN1B, JUN, GSK3B, SOS1, ARAF, MAPK8, MAP2K7, ABL2, |
| | | PIK3R1, AKT2 |
| hsa05215: Prostate | 21 | E2F3, TCF7, CREB1, RELA, CREBBP, RAF1, BAD, PIEN, CDK2, NRAS, |
| cancer | | MAPK1, EP300, KRAS, CDKN1B, BCL2, GSK3B, SOS1, ARAF, CHUK, |
| | | PIK3R1, AKT2 |
| hsa05160: Hepatitis | 28 | TRAF2, OAS1, OAS2, TNFRSF1A, KRAS, SOS1, PPP2CB, PPP2R2D, PIK3R1, |
| C | | CHUK, AKT2, TRAF3, SOCS3, RELA, RAF1, BAD, IFNAR1, NRAS, MAPK1, |
| | | IFNAR2, MAPK13, GSK3B, RIPK1, ARAF, CD81, IRF3, MAPK8, PIAS1 |
| hsa05220: Chronic | 18 | CTBP1, E2F3, RELA, CBL, RAF1, BAD, NRAS, MAPK1, CBLB, CRKL, |
| myeloid leukemia | | KRAS, CDKN1B, SOS1, ARAF, RUNX1, CHUK, PIK3R1, AKT2 |
| hsa05221: Acute | 15 | TCF7, RELA, PML, PIM1, RAF1, BAD, NRAS, MAPK1, KRAS, SOS1, ARAF, |
| myeloid leukemia | | RUNX1, PIK3R1, CHUK, AKT2 |
| hsa04664: Fc epsilon | 17 | CSF2, VAV3, MAP2K4, RAF1, VAV1, PRKCB, NRAS, MAPK1, KRAS, |
| RI signaling | | MAPK13, SOS1, RAC1, MAPK8, INPP5D, MAP2K7, PIK3R1, AKT2 |
| pathway | | |
| hsa05213: Endometrial | 14 | TCF7, RAF1, BAD, PTEN, NRAS, MAPK1, KRAS, SOS1, GSK3B, ARAF, ILK, |
| cancer | | PIK3R1, AKT2, APC |
| hsa05212: Pancreatic | 16 | E2F3, RALBP1, RELA, RAF1, SMAD3, BAD, RALGDS, MAPK1, KRAS, |
| cancer | | ARAF, RAC1, RALB, MAPK8, PIK3R1, CHUK, AKT2 |
| hsa04370: VEGF | 15 | RAF1, BAD, MAPKAPK2, PRKCB, SH2D2A, NRAS, MAPK1, KRAS, |
| signaling pathway | | MAPK13, RAC1, PPP3CB, HSPB1, NFATC2, PIK3R1, AKT2 |
| hsa04917: Prolactin | 16 | SOCS3, RELA, SOCS1, LHCGR, RAF1, NRAS, MAPK1, FOS, KRAS, |
| signaling pathway | | MAPK13, SOS1, GSK3B, MAPK8, JAK2, PIK3R1, AKT2 |
| hsa05223: Non-small | 12 | MAPK1, NRAS, E2F3, KRAS, RXRB, SOS1, ARAF, RAF1, BAD, PIK3R1, |
| cell lung cancer | | PRKCB, AKT2 |
| hsa05214: Glioma | 11 | MAPK1, NRAS, E2F3, KRAS, SOS1, ARAF, RAF1, PTEN, PIK3R1, PRKCB, |
| | | AKT2 |
| hsa05230: Central | 10 | MAPK1, NRAS, KRAS, G6PD, PFKL, RAF1, SIRT6, PIEN, PIK3R1, AKT2 |
| carbon metabolism | | |
| in cancer | | |
| hsa04730: Long-term | 9 | GNA13, MAPK1, NRAS, KRAS, PPP2CB, ARAF, RAF1, PRKCB, ITPR2 |
| depression | | |
| hsa05218: Melanoma | 10 | MAPK1, NRAS, E2F3, KRAS, ARAF, RAF1, BAD, PTEN, PIK3R1, AKT2 |
| hsa05219: Bladder | 6 | MAPK1, NRAS, E2F3, KRAS, ARAF, RAF1 |
| cancer | | |
| hsa04550: Signaling pathways | 16 | SETDB1, IL6ST, RAF1, SMAD3, LIF, PCGF5, NRAS, MAPK1, KRAS, |
| regulating pluripotency of stem cells | | MAPK13, GSK3B, JAK2, TCF3, PIK3R1, AKT2, APC |
| hsa04726: Serotonergic synapse | 8 | MAPK1, NRAS, KRAS, ARAF, RAF1, PRKACB, PRKCB, ITPR2 |
| Enrichment Score: | | |
| 1.6875120920381086 | | |
| GO: 0015035~protein disulfide | 9 | GLRX5, ENOX2, TXN2, GEER, CCS, TXNRD1, CHCHD4, GLRX2, GLRX |
| oxidoreductase activity | | |
| Redox-active center | 12 | TXNDC12, GLRX5, TXNDC11, TXN2, TMX3, TXNRD1, CHCHD4, PDIA4, |
| | | MIEN1, GLRX2, GLRX, MPST |
| domain: Glutaredoxin | 4 | GLRX5, TXNRD1, GLRX2, GLRX |
| IPR002109: Glutaredoxin | 4 | GLRX5, TXNRD1, GLRX2, GLRX |
| Enrichment Score: | | |
| 1.6621257484122156 | | |
| GO: 0006406~mRN | 24 | NUP98, FIP1L1, NUP160, SMG5, U2AF2, ALYREF, NUP93, NUP85, NUP188, |
| A export from | | CASC3, NUP155, NDC1, NUP214, UPF3B, EIF4E, DDX19A, POLDIP3, |
| nucleus | | NUP210, RBMX2, THOC6, SLU7, RANBP2, CPSF3, ALKBH5 |
| GO: 0006405~RNA | 12 | NUP214, NUP98, EIF4E, UPF3B, POLDIP3, U2AF2, ALYREF, THOC6, SLU7, |
| export from nucleus | | NUP188, CASC3, NUP155 |
| GO: 0031124~mRN | 11 | PCF11, FIP1L1, UPF3B, POLDIP3, U2AF2, ALYREF, THOC6, SLU7, CASC3, |
| A 3′-end processing | | CPSF3, CSTF1 |
| GO: 0006369~termination of RNA | 13 | PCF11, FIP1L1, UPF3B, POLDIP3, U2AF2, ALYREF, THOC6, SLU7, LSM10, |
| polymerase II transcription | | CASC3, CPSF3, CSTF1, TTF2 |
| Enrichment Score: | | |
| 1.6151522301062358 | | |
| IPR012677: Nucleotide- | 48 | RALY, RBM33, ENOX2, U2AF2, SETD1A, KIAA0430, TMEM63A, ZNF638, |
| binding, alpha-beta plait | | HNRNPLL, SART3, SF3B4, UHMK1, DNAJC17, R3HCC1L, AKAP17A, TIA1, |
| | | TARDBP, PPIL4, MSI2, RBM10, RBM28, TNRC6A, RBM22, R3HCC1, |
| | | RBM42, RBM23, SREK1, ALYREF, ELAVL1, MTHFSD, SPEN, CSTF2T, |
| | | RCAN3, PPARGC1A, LARP4B, BRAP, SAFB2, TRNAU1AP, HNRNPH2, |
| | | UPF3B, POLDIP3, RBM18, PPRC1, RBMX2, DDX50, RBM19, RNPC3, |
| | | RBM15 |
| SM00360: RRM | 37 | RALY, RBM33, ENOX2, U2AF2, KIAA0430, SETD1A, ZNF638, HNRNPLL, |
| | | SART3, SF3B4, UHMK1, TARDBP, TIA1, PPIL4, MSI2, RBM10, RBM28, |
| | | RBM22, RBM42, RBM23, SREK1, ALYREF, ELAVL1, MTHFSD, SPEN, |
| | | CSTF2T, PPARGC1A, SAFB2, TRNAU1AP, HNRNPH2, POLDIP3, RBM18, |
| | | PPRC1, RBMX2, RBM19, RNPC3, RBM15 |
| IPR000504: RNA | 38 | RALY, RBM33, ENOX2, U2AF2, KIAA0430, SETD1A, ZNF638, HNRNPLL, |
| recognition motif | | SART3, SF3B4, UHMK1, DNAJC17, TARDBP, TIA1, PPIL4, MSI2, RBM10, |
| domain | | RBM28, RBM22, RBM42, RBM23, SREK1, ALYREF, ELAVL1, MTHFSD, |
| | | SPEN, CSTF2T, PPARGC1A, SAFB2, TRNAU1AP, HNRNPH2, POLDIP3, |
| | | RBM18, PPRC1, RBMX2, RBM19, RNPC3, RBM15 |
| GO: 0000166~nucleotide | 54 | RALY, ENOX2, NT5C3A, U2AF2, KIAA0430, HINT2, HNRNPLL, ZNF638, |
| binding | | SART3, DNAJC17, AKAP17A, TIA1, TARDBP, ORC4, RBM10, R3HCC1, |
| | | RBM42, SPEN, CSTF2T, PPARGC1A, BRAP, TRNAU1AP, RBMX2, REV3L, |
| | | RBM33, SETD1A, TMEM63A, SF3B4, TRIB2, EXOSC10, R3HCC1L, CHD1L, |
| | | PPIL4, MSI2, RBM28, TNRC6A, RBM22, RBM23, MOCS2, SREK1, ALYREF, |
| | | ELAVL1, MTHFSD, RCAN3, LARP4B, SAFB2, HNRNPH2, UPF3B, POLDIP3, |
| | | PPRC1, RBM18, RBM19, RNPC3, RBM15 |
| domain: RRM | 23 | RBM22, RALY, RBM33, RBM42, ENOX2, SREK1, ALYREF, KIAA0430, |
| | | SETD1A, MTHFSD, CSTF2T, PPARGC1A, LARP4B, UHMK1, SAFB2, |
| | | DNAJC17, AKAP17A, POLDIP3, PPIL4, RBM18, PPRC1, RBMX2, TNRC6A |
| domain: RRM 2 | 19 | RBM23, U2AF2, ELAVL1, ZNF638, HNRNPLL, SPEN, SART3, SF3B4, |
| | | TRNAU1AP, HNRNPH2, TARDBP, TIA1, CPNE1, RBM19, MSI2, RNPC3, |
| | | RBM10, RBM28, RBM15 |
| domain: RRM 1 | 19 | RBM23, U2AF2, ELAVL1, ZNF638, HNRNPLL, SPEN, SART3, SF3B4, |
| | | TRNAU1AP, HNRNPH2, TARDBP, TIA1, CPNE1, RBM19, MSI2, RNPC3, |
| | | RBM10, RBM28, RBM15 |
| domain: RRM 3 | 10 | HNRNPH2, TIA1, U2AF2, CPNE1, ELAVL1, RBM19, SPEN, HNRNPLL, |
| | | RBM28, RBM15 |
| Enrichment Score: | | |
| 1.6134569375887315 | | |
| GO: 0006362~transcription | 10 | TAF1B, TAF1C, POLR1E, CCNH, POLR1A, GTF2H3, TBP, CDK7, TWISTNB, |
| elongation from RNA | | ERCC3 |
| polymerase I promoter | | |
| GO: 0006363~termination of | 10 | TAF1B, TAF1C, POLR1E, CCNH, POLR1A, GTF2H3, TBP, CDK7, TWISTNB, |
| RNA polymerase I transcription | | ERCC3 |
| GO: 0006361~transription initiation | 10 | TAF1B, TAF1C, POLR1E, CCNH, POLR1A, GTF2H3, TBP, CDK7, TWISTNB, |
| from RNA polymerase I promoter | | ERCC3 |
| GO: 0005675~holo | 5 | CCNH, GTF2F2, GTF2H3, CDK7, ERCC3 |
| TFIIH complex | | |
| GO: 0045815~positive regulation | 9 | TAF1B, TAF1C, HIST4H4, EP300, POLR1E, POLR1A, H3F3A, TBP, |
| of gene expression, epigenetic | | TWISTNB |
| Enrichment Score: | | |
| 1.611575588162036 | | |
| GO: 0000178~exosome | 10 | DIS3, ZFP36, EXOSC10, EXOSC6, EXOSC7, EXOSC5, KHSRP, EXOSC3, |
| (RNase complex) | | SKIV2L2, MPHOSPH6 |
| hsa03018: RNA | 21 | CNOT8, PAN3, PFKL, EXOSC6, EXOSC7, TTC37, EXOSC5, EXOSC3, |
| degradation | | SKIV2L2, PAPD5, EXOSC10, DIS3, BTG2, CNOT6L, DCP2, DCP1A, LSM3, |
| | | MPHOSPH6, LSM1, TOB2, ZCCHC7 |
| rRNA processing | 20 | EXOSC6, EXOSC7, EXOSC5, EXOSC3, LAS1L, SKIV2L2, PAPD5, RBFA, |
| | | RPF1, EXOSC10, DIS3, CHD7, NOL11, WDR12, NAT10, TFB1M, |
| | | MPHOSPH6, UTP20, NSUN5, DDX51 |
| GO: 0000176~nuclear exosome | 7 | DIS3, EXOSC10, EXOSC6, EXOSC7, EXOSC5, EXOSC3, MPHOSPH6 |
| (RNase complex) | | |
| GO: 0043928~exonucleolytic nuclear- | 10 | DIS3, CNOT8, EXOSC6, DCP2, EXOSC7, DCP1A, EXOSC5, EXOSC3, LSM3, |
| transcribed mRNA catabolic process | | LSM1 |
| involved in deadenylation- | | |
| dependent decay | | |
| Exosome | 6 | DIS3, EXOSC10, EXOSC6, EXOSC7, EXOSC5, EXOSC3 |
| GO: 0000175~3′-5′- | 7 | DIS3, EXOSC10, CNOT8, EXOSC7, EXOSC5, EXOSC3, ISG20L2 |
| exoribonuclease activity | | |
| GO: 0000177~cytoplasmic exosome | 5 | DIS3, EXOSC6, EXOSC7, EXOSC5, EXOSC3 |
| (RNase complex) | | |
| GO: 0034475~U4 snRNA 3′-end | 4 | EXOSC6, EXOSC7, EXOSC5, EXOSC3 |
| processing | | |
| GO: 0045006~DNA | 3 | EXOSC6, EXOSC5, EXOSC3 |
| deamination | | |
| GO: 0071034~CUT | 3 | DIS3, EXOSC10, EXOSC3 |
| catabolic process | | |
| GO: 0004532~exoribonuclease | 5 | EXOSC10, EXOSC6, EXOSC7, EXOSC5, EXOSC3 |
| activity | | |
| GO: 0034427~nuclear-transcribed | 4 | EXOSC6, EXOSC7, EXOSC5, EXOSC3 |
| mRNA catabolic process, | | |
| exonucleolytic, 3′-5′ | | |
| GO: 0071028~nuclerar mRNA | 4 | EXOSC10, EXOSC6, EXOSC7, EXOSC5 |
| surveillance | | |
| GO: 0035327-transcriptionally active | 6 | EXOSC10, PELP1, EXOSC5, TTC37, EXOSC3, CTR9 |
| chromatin | | |
| GO: 0071051~polyadenylation- | 3 | EXOSC6, EXOSC5, EXOSC3 |
| dependent snoRNA | | |
| 3′-end processing | | |
| GO: 0016075~rRNA catabolic process | 4 | DIS3, EXOSC6, EXOSC5, DEDD2 |
| IPR027408: PNPase/ | 3 | EXOSC6, EXOSC7, EXOSC5 |
| RNase PH domain | | |
| GO: 0071035~nuclear polyadenylation- | 3 | EXOSC10, EXOSC7, EXOSC3 |
| dependent rRNA catabolic process | | |
| IPR001247: Exoribo nuclease, | 3 | EXOSC6, EXOSC7, EXOSC5 |
| phosphorolytic domain 1 | | |
| IPR015847: Exoribo nuclease, | 3 | EXOSC6, EXOSC7, EXOSC5 |
| phosphorolytic domain 2 | | |
| Enrichment Score: | | |
| 1.610351623898193 | | |
| h_tnfr1Pathway: TN | 12 | TRAF2, TNFRSF1A, BAG4, LMNB2, MADD, JUN, RIPK1, MAP3K1, CASP8, |
| FR1 Signaling Pathway | | MAP2K4, MAPK8, FADD |
| h_fasPathway: FAS | 11 | LMNB2, CASP7, JUN, MAP3K1, CASP8, MAP2K4, RIPK2, MAPK8, FADD, |
| signaling pathway (CD95) | | FAF1, DAXX |
| 77.IkBa_Kinase_JNK_MEKK1 | 4 | JUN, MAP3K1, MAP2K4, MAPK8 |
| Enrichment Score: | | |
| 1.591424971929435 | | |
| IPR002219: Protein kinase C-like, | 17 | VAV3, ROCK1, ROCK2, PRKCI, PRKCH, RAF1, DGKH, MYO9B, PRKCD, |
| phorbol ester/diacylglycerol binding | | VAV1, PRKCB, PDZD8, DGKE, NSMCE1, ARAF, DGKZ, PRKD3 |
| SM00109: C1 | 16 | VAV3, ROCK1, ROCK2, PRKCI, PRKCH, RAF1, DGKH, MYO9B, VAV1, |
| | | PRKCD, PRKCB, PDZD8, DGKE, ARAF, DGKZ, PRKD3 |
| GO: 0004697~protein kinase C activity | 6 | PRKCI, PKN2, PRKCH, PRKCD, PRKD3, PRKCB |
| zinc finger region: Phorbol- | 7 | DGKE, DGKZ, PRKCH, DGKH, PRKCD, PRKD3, PRKCB |
| ester/DAG-type 2 | | |
| zinc finger region: Phorbol- | 7 | DGKE, DGKZ, PRKCH, DGKH, PRKCD, PRKD3, PRKCB |
| ester/DAG-type 1 | | |
| IPR020454: Diacylglycerol/phorbol- | 7 | ARAF, PRKCI, PRKCH, RAF1, PRKCD, PRKD3, PRKCB |
| ester binding | | |
| zinc finger region: Phorbol- | 9 | PDZD8, VAV3, ROCK1, ROCK2, ARAF, PRKCI, RAF1, MYO9B, VAV1 |
| ester/DAG-type | | |
| GO: 0030168~platelet | 17 | GNA13, VAV3, PRKCH, RAF1, DGKH, PRKCD, VAV1, SRF, FLNA, PRKCB, |
| activation | | ITPR2, PLSCR1, MAPK1, DGKE, RAC1, DGKZ, PIK3R1 |
| Enrichment Score: | | |
| 1.5519301743432432 | | |
| h_PPARgPathway: Role of PPAR- | 6 | NCOA1, NCOA2, EP300, CREBBP, PPARGC1A, MED1 |
| gamma Coactivators in Obesity and | | |
| Thermogenesis | | |
| IPR009110: Nuclear receptor coactivator, | 4 | NCOA1, NCOA2, EP300, CREBBP |
| interlocking | | |
| GO: 0035257~nuclear hormone | 7 | NCOA1, NCOA2, EP300, ACTN4, SNW1, NRIP1, MED1 |
| receptor binding | | |
| h_vdrPathway: Control of Gene | 7 | NCOA1, NCOA2, EP300, CREBBP, ACTL6A, TOP2B, MED1 |
| Expression by Vitamin D Receptor | | |
| Enrichment Score: | | |
| 1.5475594146838438 | | |
| GO: 0042800~histone methyltransferase | 8 | DPY30, ASH2L, KMT2A, KMT2C, WDR5, SETD1A, ASH1L, CXXCl |
| activity (H3-K4 specific) | | |
| hsa00310: Lysine | 14 | SETDB1, DLST, EHMT1, KMT2A, KMT2C, SETD1A, OGDH, ACAT2, |
| degradation | | COLGALT1, ALDH3A2, WHSC1L1, ASH1L, PHYKPL, ALDH9A1 |
| GO: 0051568~histone H3-K4 | 8 | DPY30, ASH2L, KMT2A, KMT2C, WDR5, SETD1A, ASH1L, CXXC1 |
| methylation | | |
| Methyltransferase | 33 | KMT2A, EZH1, KMT2C, MGMT, TRMT2B, SETD1A, VCPKMT, ASH2L, |
| | | METTL3, ASMTL, NSUN3, NSUN5, SETDB1, DNMT3A, EHMT1, METTL6, |
| | | METTL2A, METTL2B, METTL12, METTL13, KDM2A, JMJD6, TRMT13, |
| | | WHSC1L1, MTR, ASH1L, SETD6, PCMTD1, PRDM2, TH31M, SETD4, |
| | | METTL17, COMTD1 |
| GO: 0018024~histone-lysine N- | 11 | SETDB1, DPY30, EHMT1, ASH2L, KMT2A, EZH1, KMT2C, WDR5, |
| methyltransferase activity | | WHSC1L1, SETD1A, PRDM2 |
| domain: Post-SET | 6 | SETDB1, KMT2A, KMT2C, WHSC1L1, SETD1A, ASH1L |
| IPR003616: Post-SET domain | 6 | SETDB1, KMT2A, KMT2C, WHSC1L1, SETD1A, ASH1L |
| GO: 0048188~Set1C/COMPASS | 5 | DPY30, ASH2L, WDR5, SETD1A, CXXC1 |
| complex | | |
| S-adenosyl-L- | 28 | KMT2A, KMT2C, EZH1, TRMT2B, SETD1A, RSAD2, VCPKMT, METTL3, |
| methionine | | TYW1, ASMTL, LIAS, NSUN3, CDK5RAP1, NSUN5, SETDB1, DNMT3A, |
| | | EHMT1, METTL2A, METTL2B, TRMT13, WHSC1L1, MTR, ASH1L, SETD6, |
| | | PRDM2, TFB1M, SETD4, COMTD1 |
| domain: SET | 11 | SETDB1, EHMT1, KMT2A, EZH1, KMT2C, WHSC1L1, SETD1A, ASH1L, |
| | | SETD6, PRDM2, SETD4 |
| GO: 0035097~histone methyl- | 7 | DPY30, ASH2L, KMT2A, KMT2C, WDR5, SETD1A, CXXC1 |
| transferase complex | | |
| SM00317: SET | 9 | SETDB1, EHMT1, KMT2A, EZH1, KMT2C, WHSC1L1, SETD1A, ASH1L, |
| | | PRDM2 |
| IPR001214: SET | 11 | SETDB1, EHMT1, KMT2A, EZH1, KMT2C, WHSC1L1, SETD1A, ASH1L, |
| domain | | SETD6, PRDM2, SETD4 |
| GO: 0018026~peptidyl-lysine | 4 | EHMT1, KMT2A, SETD6, SETD4 |
| monomethylation | | |
| GO: 0044666~MLL3/4 complex | 4 | DPY30, ASH2L, KMT2C, WDR5 |
| zinc finger | 4 | KMT2A, KMT2C, WHSC1L1, KDM5B |
| region: PHD-type 3 | | |
| SM00508: PostSET | 4 | KMT2A, KMT2C, WHSC1L1, SETD1A |
| GO: 0034968~histone lysine | 4 | SETDB1, WHSC1L1, SETD6, PRDM2 |
| methylation | | |
| Enrichment Score: | | |
| 1.4904218811914152 | | |
| h_ceramidePathway: | 15 | TRAF2, AIFM1, RELA, MAP2K4, RAF1, FADD, BAD, MAPK1, TNFRSF1A, |
| Ceramide Signaling | | MAP3K1, BCL2, RIPK1, CASP8, MAPK8, NSMAF |
| Pathway | | |
| GO: 0071550~death-inducing signaling | 6 | TRAF2, TNFRSF1A, RIPK1, CASP8, RAF1, FADD |
| complex assembly | | |
| GO: 0097296~activation of cysteine-type | 7 | TNFRSF10A, TRAF2, RIPK1, CASP8, SMAD3, FADD, JAK2 |
| endopeptidase activity involved in | | |
| apoptotic signaling pathway | | |
| h_tnfr1Pathway: TN | 12 | TRAF2, TNFRSF1A, BAG4, LMNB2, MADD, JUN, RIPK1, MAP3K1, CASP8, |
| FR1 Signaling Pathway | | MAP2K4, MAPK8, FADD |
| GO: 0005123~death | 7 | CASP8AP2, MADD, RIPK1, CASP8, FADD, TMBIM1, FEM1B |
| receptor binding | | |
| GO: 0010803~regulation of tumor | 10 | TRAF1, TRAF2, TNFRSF1A, HIPK1, MADD, RIPK1, CASP8, PYCARD, |
| necrosis factor-mediated signaling | | RBCK1, CHUK |
| pathway | | |
| GO: 1902041~regulation of extrinsic | 7 | TNFRSF10A, TRAF2, MADD, RIPK1, CASP8, FADD, FEM1B |
| apoptotic signaling pathway via death | | |
| domain receptors | | |
| GO: 1902042~negative regulation of | 10 | TNFRSF10A, ICAM1, TRAF2, GPX1, RIPK1, CASP8, RAF1, FADD, TMBIM1, |
| extrinsic apoptotic signaling pathway | | RFFL |
| via death domain receptors | | |
| h_soddPathway: SODD/TNFR1 | 6 | TRAF2, TNFRSF1A, BAG4, RIPK1, CASP8, FADD |
| Signaling Pathway | | |
| 99.NF- | 9 | IRAK1, TRAF2, TNFRSF1A, RELA, BCL2, CREBBP, FADD, MAP3K14, |
| kB_activation | | TRAF5 |
| h_deathPathway: Induction of apoptosis | 11 | TNFRSF10A, TRAF2, XIAP, CASP7, RELA, RIPK1, BCL2, CASP8, FADD, |
| through DR3 and DR4/5 Death | | MAP3K14, CHUK |
| Receptors | | |
| h_relaPathway: Acetylation and | 7 | TNFRSF1A, EP300, RELA, RIPK1, CREBBP, FADD, CHUK |
| Deacetylation of RelA in The | | |
| Nucleus | | |
| GO: 0036462~TRAIL-activated | 3 | TNFRSF10A, CASP8, FADD |
| apoptotic signaling pathway | | |
| GO: 0045651~positive regulation of | 5 | LIF, CSF1, RIPK1, CASP8, FADD |
| macrophage differentiation | | |
| h_nfkbPathway: NF-kB Signaling | 8 | IRAK1, TNFRSF1A, RELA, RIPK1, MAP3K1, FADD, MAP3K14, CHUK |
| Pathway | | |
| GO: 2001238~positive regulation of | 7 | TRAF2, RIPK1, PML, PYCARD, RBCK1, FADD, DEDD2 |
| extrinsic apoptotic signaling pathway | | |
| 46.P13K_PTEN | 6 | TNFRSF1A, CASP7, BCL2, CASP8, FADD, BAD |
| GO: 0035666~TRIF-dependent toll-like | 7 | LY96, RIPK1, CASP8, FADD, IRF3, CHUK, TRAF3 |
| receptor signaling pathway | | |
| 40.Deg_of Chrom_DNA_TNF- | 5 | TRAF2, TNFRSF1A, RIPK1, CASP8, FADD |
| ind_apoptosis | | |
| 44.Sig_Trans_TNFR | 4 | TRAF2, RIPK1, CASP8, FADD |
| 1-DR3-DR4_DR5 | | |
| GO: 0010939~regulation of | 4 | PPIF, TRAF2, RIPK1, CASP8 |
| necrotic cell death | | |
| domain: Death | 7 | TNFRSF10A, IRAK4, TNFRSF1A, MADD, RIPK1, FADD, MALT1 |
| GO: 0031264~death-inducing | 3 | RIPK1, CASP8, FADD |
| signaling complex | | |
| GO: 0097342~ripoptosome | 3 | RIPK1, CASP8, FADD |
| 72.IAP_inter- | 5 | TNFRSF1A, XIAP, CASP7, CASP8, FADD |
| action_with_cell_death_pathways | | |
| IPR011029: Death- | 12 | TNFRSF10A, IRAK4, IRAK1, TNFRSF1A, CARD16, RIPK1, CASP8, |
| like domain | | PYCARD, RIPK2, FADD, MALT1, DEDD2 |
| 150.caspase_and_NFKB_activation | 5 | TNFRSF1A, RELA, RIPK1, CASP8, FADD |
| IPR000488: Death domain | 5 | TNFRSF10A, IRAK1, TNFRSF1A, RIPK1, FADD |
| SM00005: DEATH | 4 | TNFRSF10A, TNFRSF1A, RIPK1, FADD |
| Enrichment Score: | | |
| 1.45222941807591 | | |
| domain: VHS | 5 | STAM2, HGS, STAM, GGA1, GGA3 |
| SM00288: VHS | 5 | STAM2, HGS, STAM, GGA1, GGA3 |
| IPR002014: VHS | 5 | STAM2, HGS, STAM, GGA1, GGA3 |
| GO: 0033565~ESCR | 3 | STAM2, HGS, STAM |
| T-0 complex | | |
| repeat: UIM | 4 | RNF166, STAM2, HGS, STAM |
| IPR008942: ENTH/VHS | 7 | PCF11, CHERP, STAM2, HGS, STAM, GGA1, GGA3 |
| IPR003903: Ubiquitin | 6 | STAM2, ZFAND2B, HGS, DNAJB2, STAM, UIMC1 |
| interacting motif | | |
| GO: 0042059~negative regulation | 8 | RNF126, RNF115, TSG101, AP2A1, STAM2, CBL, HGS, STAM |
| of epidermal growth factor receptor | | |
| signaling pathway | | |
| Enrichment Score: | | |
| 1.419973773347531 | | |
| hsa04130: SNARE | 12 | SNAP29, BNIP1, STX17, BET1, VAMP5, USE1, SEC22B, BET1L, SNAP23, |
| interactions in | | VAMP2, STX10, YKT6 |
| vesicular transport | | |
| GO: 0061025~membrane fusion | 13 | SNAP29, DNM3, RABIF, UBXN2A, UBXN2B, STX17, USO1, CHP1, BET1L, |
| | | NAPA, SNAP23, VAMP2, STX10 |
| GO: 0031201~SNARE complex | 14 | SNAP29, BET1, STXBP2, NAPA, SNX4, BNIP1, STX17, VAMPS, SEC22B, |
| | | BET1L, VAMP2, SNAP23, STX10, YKT6 |
| GO: 0005484~SNAP | 10 | SNAP29, BNIP1, STX17, VAMP5, SEC22B, BET1L, SNAP23, VAMP2, STX10, |
| receptor activity | | YKT6 |
| IPR000727: Target | 6 | SNAP29, STX17, BET1, BET1L, SNAP23, STX10 |
| SNARE coiled-coil domain | | |
| domain: t-SNARE | 5 | SNAP29, STX17, BET1, BET1L, STX10 |
| coiled-coil homology | | |
| SM00397: t_SNARE | 5 | SNAP29, STX17, BET1, SNAP23, STX10 |
| GO: 0019905~syntax in binding | 9 | SNAP29, SYT11, BET1, SEC22B, NAPA, SYTL3, SNAP23, VAMP2, STX10 |
| Enrichment Score: | | |
| 1.4061663679110412 | | |
| h_41BBPathway: The 4-1BB- | 9 | TRAF2, TNFRSF9, RELA, JUN, MAP3K1, IFNG, MAPK8, CHUK, IL2 |
| dependent immune response | | |
| h_tall1Pathway: TA | 8 | TRAF2, TNFSF13B, RELA, MAPK8, MAP3K14, TRAF5, CHUK, TRAF3 |
| CI and BCMA | | |
| stimulation of B cell | | |
| immune responses. | | |
| h_stressPathway: TNF/Stress | 10 | TRAF2, TNFRSF1A, RELA, JUN, RIPK1, MAP3K1, MAP2K4, MAPK8, |
| Related Signaling | | MAP3K14, CHUK |
| h_tnfr2Pathway: TN | 8 | TRAF1, TRAF2, RELA, RIPK1, MAP3K1, MAP3K14, CHUK, TRAF3 |
| FR2 Signaling Pathway | | |
| h_nfkbPathway: NF-kB | 8 | IRAK1, TNFRSF1A, RELA, RIPK1, MAP3K1, FADD, MAP3K14, CHUK |
| Signaling Pathway | | |
| h_tollPathway: Toll-Like | 11 | IRAK1, FOS, LY96, RELA, JUN, MAP3K1, MAP2K4, MAPK8, MAP3K14, |
| Receptor Pathway | | ECSIT, CHUK |
| h_cd40Pathway: CD | 5 | RELA, MAP3K1, MAP3K14, CHUK, TRAF3 |
| 40L Signaling Pathway | | |
| h_i11rPathway: Signal | 8 | IRAK1, RELA, JUN, MAP3K1, MAPK8, MAP3K14, ECSIT, CHUK |
| transduction through IL 1R | | |
| h_rnaPathway: Double Stranded | 3 | RELA, MAP3K14, CHUK |
| RNA Induced Gene Expression | | |
| Enrichment Score: | | |
| 1.3724915407975329 | | |
| GO: 0000781~chromosome, | 12 | DPY30, TP53BP1, NSMCE1, SMC5, NSMCE2, SMC6, TINF2, SIRT2, TERF2, |
| telomeric region | | ATM, CDK2, TERF1 |
| GO: 0090398~cellular senescence | 8 | ULK3, PML, SMC5, NSMCE2, SMC6, PRKCD, SRF, TERF2 |
| GO: 0030915~Smc5-Smc6 complex | 4 | NSMCE1, SMC5, NSMCE2, SMC6 |
| Telomere | 8 | NSMCE1, SMC5, NSMCE2, SMC6, TINF2, TERF2, RAD50, TERF1 |
| GO: 0000722~telomere maintenance | 7 | RPA1, RAD51C, RFC2, SMC5, NSMCE2, SMC6, RAD50 |
| via recombination | | |
| Enrichment Score: | | |
| 1.3688179640343465 | | |
| Biological rhythms | 25 | ENOX2, KMT2A, ROCK2, CREB1, KLF10, CREBBP, PML, RORA, |
| | | PPARGC1A, PPP1CB, NRIP1, EP300, NCOA2, SIN3A, SP1, CSNK1D, |
| | | METTL3, CSNK1E, GSK3B, GFPT1, MAPK8, PRKAA1, KDM5B, FBXW11, |
| | | KDM5C |
| GO: 0042752~regulation of | 10 | CSNK1D, CSNK1E, ROCK2, CREB1, KLF10, PML, PRKAA1, MAPK8, |
| circadian rhythm | | PPARGC1A, PPP1CB |
| GO: 0032922~circadian regulation | 10 | NCOA2, CSNK1D, KMT2A, CSNK1E, GFPT1, PML, RORA, PPARGC1A, |
| of gene expression | | PPP1CB , NRIP1 |
| Enrichment Score: | | |
| 1.341911280785786 | | |
| repeat: RCC1 1 | 7 | IBTK, SERGEF, RCC2, HERC6, HERC5, HERC2, MYCBP2 |
| repeat: RCC1 2 | 7 | IBTK, SERGEF, RCC2, HERC6, HERC5, HERC2, MYCBP2 |
| repeat: RCC1 3 | 7 | IBTK, SERGEF, RCC2, HERC6, HERC5, HERC2, MYCBP2 |
| IPR000408: Regulator of | 7 | IBTK, SERGEF, RCC2, HERC6, HERC5, HERC2, MYCBP2 |
| chromosome condensation, RCC1 | | |
| IPR009091: Regulator of chromosome | 7 | IBTK, SERGEF, RCC2, HERC6, HERC5, HERC2, MYCBP2 |
| condensation 1/betalactamase- | | |
| inhibitor protein II | | |
| repeat: RCC1 5 | 6 | SERGEF, RCC2, HERC6, HERC5, HERC2, MYCBP2 |
| repeat: RCC1 4 | 6 | SERGEF, RCC2, HERC6, HERC5, HERC2, MYCBP2 |
| Cell division and chromosome | 7 | IBTK, SERGEF, RCC2, HERC6, HERC5, HERC2, MYCBP2 |
| partitioning/Cytoskeleton | | |
| repeat: RCC1 7 | 3 | SERGEF, RCC2, HERC2 |
| repeat: RCC1 6 | 3 | SERGEF, RCC2, HERC2 |
| Enrichment Score: | | |
| 1.3338979660581836 | | |
| SM00490: HELICc | 21 | BTAF1, DICER1, INO80, SKIV2L2, TTF2, CHD9, RECQL, CHD7, CHD1L, |
| | | DHX29, DDX23, DDX19A, DHX34, DDX50, DHX16, DDX10, CHD6, ERCC3, |
| | | SMARCA2, DDX51, DDX42 |
| domain: Helicase C- | 21 | BTAF1, DICER1, INO80, SKIV2L2, TTF2, CHD9, RECQL, CHD7, CHD1L, |
| terminal | | DHX29, DDX23, DDX19A, DHX34, DDX50, DHX16, DDX10, CHD6, ERCC3, |
| | | SMARCA2, DDX51, DDX42 |
| SM00487: DEXDc | 21 | BTAF1, DICER1, INO80, SKIV2L2, TTF2, CHD9, RECQL, CHD7, CHD1L, |
| | | DHX29, DDX23, DDX19A, DHX34, DDX50, DHX16, DDX10, CHD6, ERCC3, |
| | | SMARCA2, DDX51, DDX42 |
| Helicase | 25 | BTAF1, DICER1, INO80, HELZ, SKIV2L2, CHD9, MOV10, CHD1L, CHD7, |
| | | DDX23, DDX19A, DHX34, DHX16, DDX10, ERCC3, CHD6, DDX42, TTF2, |
| | | MCM6, RECQL, DHX29, GTF2F2, DDX50, SMARCA2, DDX51 |
| IPR001650: Helicase, | 21 | BTAF1, DICER1, INO80, SKIV2L2, TTF2, CHD9, RECQL, CHD7, CHD1L, |
| C-terminal | | DHX29, DDX23, DDX19A, DHX34, DDX50, DHX16, DDX10, CHD6, ERCC3, |
| | | SMARCA2, DDX51, DDX42 |
| IPR014001: Helicase, | 21 | BTAF1, DICER1, INO80, SKIV2L2, TTF2, CHD9, RECQL, CHD7, CHD1L, |
| superfamily ½, | | DHX29, DDX23, DDX19A, DHX34, DDX50, DHX16, DDX10, CHD6, ERCC3, |
| ATP-binding domain | | SMARCA2, DDX51, DDX42 |
| domain: Helicase | 21 | BTAF1, DICER1, INO80, SKIV2L2, TTF2, CHD9, RECQL, CHD7, CHD1L, |
| ATP-binding | | DHX29, DDX23, DDX19A, DHX34, DDX50, DHX16, DDX10, CHD6, ERCC3, |
| | | SMARCA2, DDX51, DDX42 |
| GO: 0004386~helicase | 17 | BTAF1, DICER1, ANXA1, HELZ, CHD9, MOV10, CHD7, DDX23, DDX19A, |
| activity | | GTF2F2, DHX34, DDX50, DDX10, ERCC3, SMARCA2, DDX51, DDX42 |
| IPR000330: SNF2- | 8 | CHD9, BTAF1, CHD7, CHD1L, INO80, CHD6, SMARCA2, TTF2 |
| related | | |
| GO: 0008026~ATP- | 7 | RECQL, CHD1L, DDX23, DHX29, DHX16, CHD6, TTF2 |
| dependent helicase | | |
| activity | | |
| IPR011545: DNA/RNA | 12 | RECQL, DDX23, DHX29, DDX19A, DICER1, DHX34, DDX50, DHX16, |
| helicase, DEAD/DEAH box | | SKIV2L2, DDX10, DDX51, DDX42 |
| type, N-terminal | | |
| IPR002464: DNA/RNA helicase, | 5 | CHD1L, DHX29, DHX16, CHD6, TTF2 |
| ATP-dependent, DEAH- | | |
| box type, conserved site | | |
| short sequence | 7 | CHD9, CHD7, CHD1L, DHX29, DHX16, CHD6, TTF2 |
| motif: DEAH box | | |
| GO: 0004004~ATP- | 9 | DDX23, DHX29, DDX19A, DHX34, DDX50, DHX16, DDX10, DDX51, |
| dependent RNA | | DDX42 |
| helicase activity | | |
| Enrichment Score: | | |
| 1.3229064054856214 | | |
| domain: PCI | 7 | PSMD13, PSMD12, PCID2, COPS7A, PSMD3, COPS7B, COPS8 |
| IPR000717: Proteasome | 6 | PSMD13, PSMD12, PCID2, COPS7A, PSMD3, COPS7B |
| component (PCI) domain | | |
| GO: 0022624~proteasome | 6 | PSMD13, PSMC5, PSMD12, PSMC2, PSMD3, PSMD5 |
| accessory complex | | |
| SM00088: PINT | 5 | PSMD13, PSMD12, COPS7A, PSMD3, COPS7B |
| GO: 0008541~proteasome regulatory | 3 | PSMD13, PSMD12, PSMD3 |
| particle, lid subcomplex | | |
| Enrichment Score: | | |
| 1.318551447098944 | | |
| Iron-sulfur | 13 | GLRX5, TYW1, NDUFV2, IREB2, RSAD2, LIAS, UQCRFS1, CDK5RAP1, |
| | | CIAPIN1, NDUFS1, PPAT, GLRX2, REV3L |
| 2Fe-2S | 6 | GLRX5, NDUFV2, UQCRFS1, CIAPIN1, NDUFS1, GLRX2 |
| GO: 0051537~2 iron, 2 sulfur | 6 | GLRX5, NDUFV2, UQCRFS1, CIAPIN1, NDUFS1, GLRX2 |
| cluster binding | | |
| Enrichment Score: | | |
| 1.309987729796792 | | |
| IPR016159: Cullin | 6 | CUL3, CUL2, EXOC7, CACUL1, VPS51, CUL4B |
| repeat-like- | | |
| containing domain | | |
| IPR001373: Cullin, | 5 | CUL3, CUL2, CACUL1, CUL9, CUL4B |
| N-terminal | | |
| IPR016157: Cullin, | 4 | CUL3, CUL2, CUL9, CUL4B |
| conserved site | | |
| SM00884: SM00884 | 4 | CUL3, CUL2, CUL9, CUL4B |
| IPR019559: Cullin | 4 | CUL3, CUL2, CUL9, CUL4B |
| protein, neddylation | | |
| domain | | |
| IPR016158: Cullin | 4 | CUL3, CUL2, CUL9, CUL4B |
| homology | | |
| GO: 0031461~cullin- | 4 | CUL3, CUL2, CUL9, CAND1 |
| RING ubiquitin | | |
| ligase complex | | |
| cross-link: Glycyl | 3 | CUL3, CUL2, CUL4B |
| lysine isopeptide | | |
| (Lys-Gly) | | |
| (interchain with G- | | |
| Cter in NEDD8) | | |
| SM00182: CULLIN | 3 | CUL3, CUL2, CUL4B |
| Enrichment Score: | | |
| 1.3095823857533413 | | |
| zinc finger | 6 | YAF2, RYBP, RBCK1, MDM4, RBM10, TAB3 |
| region: RanBP2-type | | |
| IPR001876: Zinc | 7 | YAF2, RYBP, RBCK1, MDM4, RANBP2, RBM10, TAB3 |
| finger, RanBP2-type | | |
| SM00547: ZnF_RBZ | 6 | YAF2, RYBP, RBCK1, RANBP2, RBM10, TAB3 |
| Enrichment Score: | | |
| 1.3076994990472324 | | |
| active site: Glycyl | 18 | UBE2A, UBE2Z, UBE2G1, HERC6, UBE2J1, HERC5, BIRC6, UBA5, HERC2, |
| thioester | | UBE2J2, UBE2R2, UBE2D4, UBA3, UBE2W, SMURF2, HECTD4, HECTD1, |
| intermediate | | UBE2E1 |
| repeat: RCC1 5 | 6 | SERGEF, RCC2, HERC6, HERC5, HERC2, MYCBP2 |
| repeat: RCC1 4 | 6 | SERGEF, RCC2, HERC6, HERC5, HERC2, MYCBP2 |
| domain: HECT | 6 | HERC6, HERC5, HECTD4, SMURF2, HERC2, HECTD1 |
| SM00119: HECTc | 6 | HERC6, HERC5, HECTD4, SMURF2, HERC2, HECTD1 |
| IPR000569: HECT | 6 | HERC6, HERC5, HECTD4, SMURF2, HERC2, HECTD1 |
| Enrichment Score: | | |
| 1.303394556655991 | | |
| h_metPathway: Signaling of | 13 | MAP4K1, RAF1, ITGB1, PTEN, FOS, MAPK1, CRKL, SOS1, JUN, MAPK8, |
| Hepatocyte Growth Factor Receptor | | RAPGEF1, RASA1, PIK3R1 |
| h_integrinPathway: | 11 | MAPK1, CAPNS1, CRKL, ROCK1, JUN, SOS1, RAF1, MAPK8, ZYX, |
| Integrin Signaling Pathway | | RAPGEF1, ITGB1 |
| hsa04510: Focal | 27 | XIAP, ITGB1, PTEN, SOS1, BCL2, ILK, RAC1, ZYX, RAPGEF1, PIK3R1, |
| adhesion | | AKT2, PARVG, VAV3, ACTN4, ROCK1, ROCK2, RAF1, BAD, VAV1, |
| | | PPP1CB, FLNA, PRKCB, MAPK1, CRKL, JUN, GSK3B, MAPK8 |
| Enrichment Score: | | |
| 1.3018570193497914 | | |
| GO: 0000175~3′-5′- | 7 | DIS3, EXOSC10, CNOT8, EXOSC7, EXOSC5, EXOSC3, ISG20L2 |
| exoribonuclease activity | | |
| GO: 0090503~RNA phosphodiester | 8 | DIS3, EXOSC10, CNOT8, PAN3, CNOT6L, DCP2, EXOSC5, ISG20L2 |
| bond hydrolysis, exonucleolytic | | |
| Exonuclease | 9 | DIS3, EXOSC10, RAD1, CNOT8, CNOT6L, AEN, REXO1, ERI3, ISG20L2 |
| Enrichment Score: | | |
| 1.3001157372818273 | | |
| domain: J | 12 | DNAJC17, DNAJC15, DNAJC16, DNAJB9, DNAJC11, SACS, DNAJC5, |
| | | DNAJB2, HSCB, DNAJB6, DNAJC30, GAK |
| IPR001623: DnaJ | 12 | DNAJC17, DNAJC15, DNAJC16, DNAJB9, DNAJC11, SACS, DNAJC5, |
| domain | | DNAJB2, HSCB, DNAJB6, DNAJC30, GAK |
| SM00271: DnaJ | 10 | DNAJC17, DNAJC15, DNAJC16, DNAJB9, DNAJC11, DNAJC5, DNAJB2, |
| | | DNAJB6, DNAJC30, GAK |
| IPR018253: DnaJ | 6 | DNAJC16, DNAJB9, DNAJC11, DNAJC5, DNAJB2, DNAJB6 |
| domain, conserved site | | |
| Enrichment Score: | | |
| 1.296835482359286 | | |
| hsa04130: SNARE | 12 | SNAP29, BNIP1, STX17, BET1, VAMP5, USE1, SEC22B, BET1L, SNAP23, |
| interactions in vesicular transport | | VAMP2, STX10, YKT6 |
| GO: 0005484~SNAP | 10 | SNAP29, BNIP1, STX17, VAMP5, SEC22B, BET1L, SNAP23, VAMP2, STX10, |
| receptor activity | | YKT6 |
| domain: v-SNARE | 4 | VAMP5, SEC22B, VAMP2, YKT6 |
| coiled-coil homology | | |
| IPR001388: Synaptobrevin | 4 | VAMP5, SEC22B, VAMP2, YKT6 |
| GO: 0000149~SNARE binding | 7 | GABARAPL2, STX17, VAMPS, SEC22B, SNAPIN, VAMP2, YKT6 |
| Enrichment Score: | | |
| 1.2920732660982135 | | |
| GO: 0016578~histone deubiquitination | 6 | TAF10, USP3, USP22, TRRAP, USP16, USP34 |
| zinc finger region: UBP-type | 5 | USP3, USP5, USP22, USP16, BRAP |
| IPR001607: Zinc finger, UBP-type | 5 | USP3, USP5, USP22, USP16, BRAP |
| SM00290: ZnF_UBP | 4 | USP3, USP5, USP16, BRAP |
| Enrichment Score: | | |
| 1.271806287569803 | | |
| GO: 0042809~vitamin | 6 | MED16, MED17, SNW1, MED13, TOB2, MED1 |
| D receptor binding | | |
| GO: 0046966~thyroid hormone | 8 | HMGN3, MED16, MED17, JMJD1C, MED13, GTF2B, MEDI, ZNHIT3 |
| receptor binding | | |
| GO: 0016592~mediator complex | 9 | MED31, MED19, MED16, MED8, MED17, MED11, MED13, MED13L, MED1 |
| GO: 0030518~intracellular steroid | 4 | MED16, MED17, MED13, MEDI |
| hormone receptor signaling pathway | | |
| Enrichment Score: | | |
| 1.271330707752596 | | |
| zinc finger region: TFIIB-type | 3 | BRF1, BRF2, GTF2B |
| IPR000812: Transcription factor TFIIB | 3 | BRF1, BRF2, GTF2B |
| IPR013137: Zinc finger, TFEB-type | 3 | BRF1, BRF2, GTF2B |
| IPR013763: Cyclin-like | 9 | CCNT2, BRF1, BRF2, CCNH, CCNT1, CCNG1, CCNG2, GTF2B, CASD1 |
| Enrichment Score: | | |
| 1.269935983244461 | | |
| h_tcrPathway: T Cell | 18 | PTPN7, RELA, CD247, MAP2K4, RAF1, VAV1, PRKCB, FOS, JUN, MAP3K1, |
| Receptor Signaling Pathway | | SOS1, RAC1, ZAP70, PPP3CB, MAPK8, NFATC2, RASA1, PIK3R1 |
| h_gleevecPathway: Inhibition of | 11 | FOS, CRKL, JUN, SOS1, MAP3K1, MAP2K4, RAF1, MAPK8, JAK2, BAD, |
| Cellular Proliferation by Gleevec | | PIK3R1 |
| h_fcer1Pathway: Fc Epsilon Receptor | 14 | MAP2K4, RAF1, VAV1, PRKCB, MAPK1, FOS, MAP3K1, SOS1, JUN, |
| I Signaling in Mast Cells | | PPP3CB, MAPK8, NFATC2, MAP2K7, PIK3R1 |
| h_egfPathway: EGF | 11 | FOS, JUN, SOS1, MAP3K1, MAP2K4, RAF1, MAPK8, SRF, PIK3R1, RASA1, |
| Signaling Pathway | | PRKCB |
| h_metPathway: Signaling of Hepatocyte | 13 | MAP4K1, RAF1, ITGB1, PTEN, FOS, MAPK1, CRKL, SOS1, JUN, MAPK8, |
| Growth Factor Receptor | | RAPGEF1, RASA1, PIK3R1 |
| h_pdgfPathway: PD GF Signaling | 11 | FOS, JUN, SOS1, MAP3K1, MAP2K4, RAF1, MAPK8, SRF, PIK3R1, RASA1, |
| Pathway | | PRKCB |
| h_arenrf2Pathway: Oxidative Stress | 9 | MAPK1, FOS, JUN, CREB1, AKR7A2, MAPK8, KEAP1, NFE2L2, PRKCB |
| Induced Gene Expression Via Nrf2 | | |
| h_bcrPathway: BCR | 12 | FOS, JUN, SOS1, MAP3K1, RAC1, PPP3CB, RAF1, MAPK8, CD79B, |
| Signaling Pathway | | NFATC2, VAV1, PRKCB |
| h_pyk2Pathway: Links between | 10 | MAPK1, CRKL, JUN, SOS1, MAP3K1, RAC1, MAP2K4, RAF1, MAPK8, |
| Pyk2 and Map Kinases | | PRKCB |
| h_insulinPathway: Insulin Signaling | 8 | FOS, JUN, SOS1, RAF1, MAPK8, SRF, PIK3R1, RASA1 |
| Pathway | | |
| h_igf1Pathway: IGF-1 Signaling | 8 | FOS, JUN, SOS1, RAF1, MAPK8, SRF, PIK3R1, RASA1 |
| Pathway | | |
| hsa04912: GnRH | 17 | ADCY7, MAP2K4, RAF1, PRKCD, PRKCB, ITPR2, NRAS, MAPK1, KRAS, |
| signaling pathway | | MAP3K3, MAPK13, JUN, MAP3K1, SOS1, MAPK8, PRKACB, MAP2K7 |
| h_at1rPathway: Angiotensin II | 10 | MAPK1, MEF2A, JUN, SOS1, MAP3K1, RAC1, MAP2K4, RAF1, MAPK8, |
| mediated activation of JNK Pathway | | PRKCB |
| via Pyk2 dependent signaling | | |
| h_malPathway: Role of MAL in Rho- | 7 | MAPK1, ROCK1, MAP3K1, RAC1, RAF1, MAPK8, SRF |
| Mediated Activation of SRF | | |
| h_tpoPathway: TPO Signaling Pathway | 8 | FOS, JUN, SOS1, RAF1, JAK2, PIK3R1, RASA1, PRKCB |
| 102.Cholesterol_Stress_Response | 4 | MAP3K1, RAC1, RAF1, MAPK8 |
| 67.Ikaros_and_signaling_inhibitors | 9 | MAPK1, FOS, CD8A, JUN, SOS1, RAF1, NFATC2, VAV1, IL2 |
| h_il6Pathway: IL 6 signaling pathway | 7 | FOS, IL6ST, JUN, SOS1, RAF1, JAK2, SRF |
| h_cdmacPathway: Cadmium induces | 6 | MAPK1, FOS, RELA, JUN, RAF1, PRKCB |
| DNA synthesis and proliferation in | | |
| macrophages | | |
| h_ccr5Pathway: Pertussis toxin- | 6 | FOS, CXCR4, JUN, MAPK8, CCL4, PRKCB |
| insensitive CCR5 Signaling in | | |
| Macrophage | | |
| h_il2Pathway: IL 2 signaling pathway | 7 | FOS, IL2RB, JUN, SOS1, RAF1, MAPK8, IL2 |
| h_crebPathway: Transcription factor | 8 | MAPK1, PRKAR2A, CREB1, SOS1, RAC1, PRKACB, PIK3R1, PRKCB |
| CREB and its extracellular signals | | |
| h_ghPathway: Growth Hormone | 8 | MAPK1, SOS1, SOCS1, RAF1, JAK2, SRF, PIK3R1, PRKCB |
| Signaling Pathway | | |
| 68.Mitogen_signaling_in_growth_con- | 4 | MAPK1, SOS1, MAP3K1, RAF1 |
| trol | | |
| GO: 0051090~regulation of sequence- | 6 | MAPK1, FOS, TAF6, CREBZF, JUN, MAPK8 |
| specific DNA binding transcription | | |
| factor activity | | |
| h_epoPathway: EPO Signaling Pathway | 6 | FOS, JUN, SOS1, RAF1, MAPK8, JAK2 |
| 77.IkBa_Kinase_JNK_MEKK1 | 4 | JUN, MAP3K1, MAP2K4, MAPK8 |
| h_ngfPathway: Nerve growth factor | 6 | FOS, JUN, SOS1, RAF1, MAPK8, PIK3R1 |
| pathway (NGF) | | |
| 3.T_cell_receptor | 7 | FOS, JUN, CTLA4, ZAP70, MAPK8, VAV1, IL2 |
| GO: 0035994~response | 4 | FOS, RELA, JUN, RAF1 |
| to muscle stretch | | |
| 100.MAPK_signaling_cascades | 7 | MAPK1, MAP3K3, MAP3K1, MAP2K4, RAF1, MAPK8, MAP2K7 |
| h_il3Pathway: IL 3 signaling pathway | 4 | FOS, SOS1, RAF1, JAK2 |
| GO: 0061029~eyelid development in | 3 | JUN, SOS1, SRF |
| camem-type eye | | |
| h_trkaPathway: Trka Receptor | 3 | SOS1, PIK3R1, PRKCB |
| Signaling Pathway | | |
| Enrichment Score: | | |
| 1.263372828431808 | | |
| h_tall1Pathway: TACI and BCMA | 8 | TRAF2, TNFSF13B, RELA, MAPK8, MAP3K14, TRAF5, CHUK, TRAF3 |
| stimulation of B cell immune responses. | | |
| h_tnfr2Pathway: TNFR2 Signaling | 8 | TRAF1, TRAF2, RELA, RIPK1, MAP3K1, MAP3K14, CHUK, TRAF3 |
| Pathway | | |
| GO: 0031996~thioesterase binding | 6 | TRAF1, TRAF2, RAC1, ARF6, TRAF5, TRAF3 |
| IPR012227: TNF receptor-associated | 4 | TRAF1, TRAF2, TRAF5, TRAF3 |
| factor TRAF | | |
| PIRSF015614: TNF receptor-associated | 4 | TRAF1, TRAF2, TRAF5, TRAF3 |
| factor (TRAF) | | |
| domain: MATH | 5 | TRAF1, TRAF2, TRAF5, SPOP, TRAF3 |
| SM00061: MATH | 5 | TRAF1, TRAF2, TRAF5, SPOP, TRAF3 |
| IPR002083: MATH | 5 | TRAF1, TRAF2, TRAF5, SPOP, TRAF3 |
| zinc finger region: TRAF-type 1 | 3 | TRAF2, TRAF5, TRAF3 |
| zinc finger region: TRAF-type 2 | 3 | TRAF2, TRAF5, TRAF3 |
| GO: 0005164~tumor necrosis factor | 7 | TRAF1, TRAF2, TRAP1, TNFSF13B, CASP8, FADD, TRAF3 |
| receptor binding | | |
| GO: 0035631~CD40 receptor complex | 4 | TRAF2, TRAF5, CHUK, TRAF3 |
| IPR008974: TRAF-like | 6 | TRAF1, TRAF2, XAF1, TRAF5, SPOP, TRAF3 |
| IPR001293: Zinc finger, TRAF-type | 4 | TRAF2, XAF1, TRAF5, TRAF3 |
| 51.CD40_and_EBV | 4 | TRAF1, TRAF2, MAPK8, TRAF3 |
| IPR018957: Zinc finger, C3HC4 | 5 | MKRN1, TRAF2, PEX2, TRAF5, TRAF3 |
| RING-type | | |
| Enrichment Score: | | |
| 1.261550763230275 | | |
| DNA-binding region: A.T hook 2 | 5 | PDS5B, KMT2A, ASH1L, MECP2, BAZ2A |
| DNA-binding region: A.T hook 1 | 5 | PDS5B, KMT2A, ASH1L, MECP2, BAZ2A |
| DNA-binding region: A.T hook 3 | 4 | PDS5B, KMT2A, ASH1L, BAZ2A |
| Enrichment Score: | | |
| 1.2578929146832885 | | |
| Redox-active center | 12 | TXNDC12, GLRX5, TXNDC11, TXN2, TMX3, TXNRD1, CHCHD4, PDIA4, |
| | | MIEN1, GLRX2, GLRX, MPST |
| GO: 0045454~cell | 17 | TMX2, GLRX5, TXN2, AIFM1, TMX3, TXNDC9, PDIA4, GLRX2, GPX1, |
| redox homeostasis | | TXNDC12, TXNDC11, DNAJC16, KRIT1, TXNRD1, NFE2L2, SCO2, GLRX |
| IPR012336: Thioredoxin-like fold | 24 | TMX2, GLRX5, TXN2, MRPS25, TMX3, TXNDC9, PDIA4, CLIC1, AAED1, |
| | | MIEN1, GLRX2, GPX1, SH3BGRL, TXNDC12, TXNDC11, DNAJC16, |
| | | EEF1E1, NDUFV2, FAF2, TXNRD1, FAF1, GPX7, SCO2, GLRX |
| IPR013766: Thioredoxin domain | 9 | TMX2, TXNDC12, TXNDC11, DNAJC16, TXN2, TMX3, TXNDC9, PDIA4, |
| | | SCO2 |
| domain: Thioredoxin | 6 | TMX2, DNAJC16, TXN2, TMX3, TXNDC9, SCO2 |
| GO: 0005623~cell | 13 | TMX2, TXNDC11, DNAJC16, AIFM1, TXN2, NELL2, TMX3, MCPH1, |
| | | SLC41A1, TXNRD1, PDIA4, XCL1, GLRX2 |
| Enrichment Score: | | |
| 1.2512406598500696 | | |
| GO: 0035267~NuA4 histone | 6 | ING3, ACTL6A, TRRAP, KAT5, MRGBP, BRD8 |
| acetyltransferase complex | | |
| GO: 0040008~regulation of growth | 12 | ING3, ING2, ENOX2, SOCS3, NELL2, SOCS1, CD81, IFNG, ACTL6A, KAT5, |
| | | MRGBP, BRD8 |
| Growth regulation | 11 | ING3, ING2, ENOX2, TSG101, SOCS3, SOCS1, IFNG, ACTL6A, KAT5, |
| | | MRGBP, BRD8 |
| Enrichment Score: | | |
| 1.2449747006931964 | | |
| GO: 0008536~Ran GTPase binding | 9 | XPO6, IPO7, RANGAP1, NUTF2, RANGRF, RANBP2, TNPO2, XPO7, TNPO1 |
| domain: Importin N-terminal | 5 | XPO6, IPO7, TNPO2, XPO7, TNPO1 |
| SM00913: SM00913 | 5 | XPO6, IPO7, TNPO2, XPO7, TNPO1 |
| IPR001494: Importin-beta, N-terminal | 5 | XPO6, IPO7, TNPO2, XPO7, TNPO1 |
| Enrichment Score: | | |
| 1.219152476389818 | | |
| SM00455: RBD | 4 | TIAM1, ARAF, RAF1, RGS14 |
| IPR003116: Raf-like Ras-binding | 4 | TIAM1, ARAF, RAF1, RGS14 |
| GO: 0005057~receptor signaling | 9 | BAG4, IFITM1, TIAM1, IL4R, ARAF, RAF1, NSMAF, DAXX, RGS14 |
| protein activity | | |
| domain: RBD | 3 | TIAM1, ARAF, RAF1 |
| Enrichment Score: | | |
| 1.2191033373017977 | | |
| GO: 0000974~Prp19 complex | 6 | RBM22, PRPF19, CRNKL1, U2AF2, ISY1, XAB2 |
| GO: 0071014~post-mRNA release | 3 | CRNKL1, ISY1, XAB2 |
| spliceosomal complex | | |
| GO: 0071012~catalytic step 1 | 3 | CRNKL1, ISY1, XAB2 |
| spliceosome | | |
| Enrichment Score: | | |
| 1.2188361598202275 | | |
| IPR000814: TATA-box binding protein | 3 | TBPL2, TBP, TBPL1 |
| GO: 0006352~DNA-templated | 9 | TBPL2, TAF10, TAF13, HIST4H4, BRF2, TAF6, TBP, GTF2B, TBPL1 |
| transcription, initiation | | |
| IPR012295: Beta2-adaptin/TBP, C- | 3 | TBPL2, TBP, TBPL1 |
| terminal domain | | |
| Enrichment Score: | | |
| 1.2120424551901947 | | |
| GO: 0004697~protein kinase C activity | 6 | PRKCI, PKN2, PRKCH, PRKCD, PRKD3, PRKCB |
| GO: 0034351~negative regulation of | 4 | TRAF2, PRKCI, PRKCH, PRKCD |
| glial cell apoptotic process | | |
| IPR020454: Diacylglycerol/phorbol- | 7 | ARAF, PRKCI, PRKCH, RAF1, PRKCD, PRKD3, PRKCB |
| ester binding | | |
| domain.AGC-kinase | 11 | RPS6KA3, ROCK1, ROCK2, PRKCI, PKN2, PRKCH, PRKACB, PRKCD, |
| C-terminal | | LATS1, PRKCB, AKT2 |
| SM00133: S_TK_X | 10 | RPS6KA3, ROCK1, ROCK2, PRKCI, PKN2, PRKCH, PRKACB, PRKCD, |
| | | PRKCB, AKT2 |
| IPR000961: AGC- | 11 | RPS6KA3, ROCK1, ROCK2, PRKCI, PKN2, PRKCH, PRKACB, PRKCD, |
| kinase, C-terminal | | LATS1, PRKCB, AKT2 |
| IPR017892: Protein | 7 | RPS6KA3, PRKCI, PKN2, PRKCH, PRKCD, PRKCB, AKT2 |
| kinase, C-terminal | | |
| Enrichment Score: | | |
| 1.1705954418260636 | | |
| IPR027267: Arfaptin homology (AH) | 9 | ICA1, SH3GLB2, ACAP1, ACAP2, ARFIP2, ASAP1, ARHGAP17, BIN3, BIN1 |
| domain/BAR domain | | |
| domain: BAR | 6 | SH3GLB2, ACAP1, ACAP2, ARHGAP17, BIN3, BIN1 |
| SM00721: BAR | 4 | SH3GLB2, ARHGAP17, BIN3, BIN1 |
| IPR004148: BAR domain | 4 | SH3GLB2, ARHGAP17, BIN3, BIN1 |
| Enrichment Score: | | |
| 1.167006763733174 | | |
| IPR006689: Small GTPase superfamily, | 9 | ARL2, ARL5A, ARF4, ARF6, ARL8B, SAR1B, ARL4C, ARL3, ARL4A |
| ARF/SAR type | | |
| IPR024156: Small GTPase superfamily, | 8 | ARL2, ARL5A, ARF4, ARF6, ARL8B, ARL4C, ARL3, ARL4A |
| ARF type | | |
| binding site: GTP; via amide nitrogen | 3 | ARL2, ARL5A, ARL3 |
| Enrichment Score: | | |
| 1.1575764161081228 | | |
| GO: 0009165~nucleotide | 6 | DCTD, DCK, DGUOK, PRPS2, PRPS1, PRPSAP2 |
| biosynthetic process | | |
| Nucleotide biosynthesis | 4 | DCTD, PRPS2, PRPS1, PRPSAP2 |
| IPR005946: Ribose phosphate | 3 | PRPS2, PRPS1, PRPSAP2 |
| diphosphokinase | | |
| GO: 0004749~ribose phosphate | 3 | PRPS2, PRPS1, PRPSAP2 |
| diphosphokinase activity | | |
| IPR000836: Phosphoribosyltransferase | 4 | PRPS2, PPAT, PRPS1, PRPSAP2 |
| domain | | |
| Enrichment Score: | | |
| 1.1356123741363102 | | |
| domain.ARID | 5 | ARID4A, ARID5A, ARID1B, KDM5B, KDM5C |
| SM00501: BRIGHT | 5 | ARID4A, ARID5A, ARID1B, KDM5B, KDM5C |
| IPR001606: ARID/BRIGHT DNA- | 5 | ARID4A, ARID5A, ARID1B, KDM5B, KDM5C |
| binding domain | | |
| Enrichment Score: | | |
| 1.1355492828606781 | | |
| hsa00020: Citrate cycle (TCA cycle) | 9 | SDHA, DLST, IDH3G, SDHC, IDH2, PCK2, OGDH, MDH1, FH |
| Tricarboxylic acid cycle | 7 | SDHA, DLST, IDH3G, SDHC, IDH2, MDH1, FH |
| GO: 0006099~tricarboxylic acid cycle | 8 | SDHA, DLST, IDH3G, SDHC, IDH2, OGDH, MDH1, FH |
| GO: 0006734~NADH metabolic process | 4 | DLST, IDH3G, OGDH, MDH1 |
| hsa01200: Carbon | 18 | DLST, ME2, PFKL, GLUD2, OGDH, ACAT2, SDHA, PGP, G6PD, IDH3G, |
| metabolism | | SDHC, PHGDH, IDH2, PCCB, PRPS2, MDH1, FH, PRPS1 |
| GO: 0006103~2-oxoglutarate | 4 | DLST, IDH3G, IDH2, OGDH |
| metabolic process | | |
| Enrichment Score: | | |
| 1.1310275733652524 | | |
| hsa05212: Pancreatic | 16 | E2F3, RALBP1, RELA, RAF1, SMAD3, BAD, RALGDS, MAPK1, KRAS, |
| cancer | | ARAF, RAC1, RALB, MAPK8, PIK3R1, CHUK, AKT2 |
| h_rasPathway: Ras Signaling Pathway | 8 | RALBP1, RELA, RAC1, RAF1, BAD, PIK3R1, CHUK, RALGDS |
| h_raccycdPathway: Influence of Ras | 8 | MAPK1, CDKN1B, RELA, RAC1, RAF1, PIK3R1, CHUK, CDK2 |
| and Rho proteins on G1 to S Transition | | |
| h_aktPathway: AKT Signaling Pathway | 4 | RELA, BAD, PIK3R1, CHUK |
| Enrichment Score: | | |
| 1.1043180593977402 | | |
| zinc finger region: C4-type | 16 | ARFGAP2, TRIP4, AGFG2, GTF2H3, ASAP1, POLR2B, RPA1, BRPF1, |
| | | SMAP1, ASH2L, ACAP1, MLLT10, ACAP2, MLLT6, ARAP2, REV3L |
| domain.Arf-GAP | 7 | ARFGAP2, SMAP1, AGFG2, ACAP1, ACAP2, ASAP1, ARAP2 |
| SM00105: ArfGap | 7 | ARFGAP2, SMAP1, AGFG2, ACAP1, ACAP2, ASAP1, ARAP2 |
| IPR001164: Arf GTPase | 7 | ARFGAP2, SMAP1, AGFG2, ACAP1, ACAP2, ASAP1, ARAP2 |
| activating protein | | |
| Enrichment Score: | | |
| 1.0984383664834843 | | |
| GO: 0060334~regulation of interferon- | 6 | SOCS3, SOCS1, IFNG, JAK2, PIAS1, IFNGR2 |
| gamma-mediated signaling pathway | | |
| h_tidPathway: Chaperones modulate | 6 | TNFRSF1A, RELA, IFNG, HSPA1A, JAK2, IFNGR2 |
| interferon Signaling Pathway | | |
| h_ifngPathway: IFN gamma | 3 | IFNG, JAK2, IFNGR2 |
| signaling pathway | | |
| Enrichment Score: | | |
| 1.0873412249855727 | | |
| GO: 0001844~protein insertion into | 4 | MOAP1, BBC3, BAD, BCL2L11 |
| mitochondrial membrane involved | | |
| in apoptotic signaling pathway | | |
| GO: 2001244~positive regulation of | 8 | FIS1, BBC3, BCL2, PRKRA, BAD, BCL2L11, PLAGL2, BCAP31 |
| intrinsic apoptotic signaling pathway | | |
| short sequence motif: BH3 | 5 | MCL1, BBC3, BCL2, BAD, BCL2L11 |
| GO: 1900740~positive regulation of | 7 | BBC3, BCL2, CASP8, MAPK8, GZMB, BAD, BCL2L11 |
| protein insertion into mitochondrial | | |
| membrane involved in apoptotic | | |
| signaling pathway | | |
| Enrichment Score: | | |
| 1.0840410197958643 | | |
| TPR repeat | 29 | MAU2, UTY, FKBP5, FEM1B, FEM1A, STUB1, FIS1, FICD, KLC1, VPS13A, |
| | | RANBP2, GTF3C3, TTC31, TTC14, WDTC1, ZC3H7A, ZC3H7B, TTC37, |
| | | CDC23, NAA25, CLUH, CDC27, SGTB, TTC17, CTR9, IFIT3, RPAP3, IFIT5, |
| | | EMC2 |
| IPR011990: Tetratricopeptide- | 37 | CRNKL1, MAU2, FKBP5, UTY, NAPA, HELZ, TRRAP, CLTC, SART3, |
| like helical | | STUB1, FEM1A, XAB2, FIS1, FICD, KLC1, PSMD3, RANBP2, GTF3C3, |
| | | TTC31, TTC14, WDTC1, ZC3H7A, ZC3H7B, SMG5, TTC37, NAA25, CDC23, |
| | | CLUH, VPS41, TTC17, CDC27, SGTB, CTR9, IFIT3, RPAP3, IFIT5, EMC2 |
| IPR013026: Tetratricopeptide | 24 | TTC31, TTC14, WDTC1, CRNKL1, ZC3H7B, UTY, FKBP5, TTC37, NAA25, |
| repeat-containing domain | | CDC23, CDC27, TTC17, XAB2, SGTB, STUB1, CTR9, IFIT3, RPAP3, FICD, |
| | | KLC1, IFIT5, EMC2, RANBP2, GTF3C3 |
| SM00028: TPR | 22 | TTC31, TTC14, WDTC1, MAU2, ZC3H7B, UTY, FKBP5, TTC37, CDC23, |
| | | CDC27, TTC17, STUB1, XAB2, SGTB, CTR9, IFIT3, RPAP3, KLC1, IFIT5, |
| | | EMC2, RANBP2, GTF3C3 |
| repeat: TPR 1 | 26 | MAU2, FKBP5, UTY, STUB1, FEM1A, FICD, KLC1, VPS13A, GTF3C3, |
| | | TTC31, WDTC1, TTC14, ZC3H7A, ZC3H7B, TTC37, CDC23, NAA25, CLUH, |
| | | SGTB, TTC17, CDC27, CTR9, RPAP3, IFIT3, IFIT5, EMC2 |
| repeat: TPR 2 | 26 | MAU2, FKBP5, UTY, STUB1, FEM1A, FICD, KLC1, VPS13A, GTF3C3, |
| | | TTC31, WDTC1, TTC14, ZC3H7A, ZC3H7B, TTC37, CDC23, NAA25, CLUH, |
| | | SGTB, TTC17, CDC27, CTR9, RPAP3, IFIT3, IFIT5, EMC2 |
| IPR019734: Tetratricopeptide | 23 | TTC31, TTC14, WDTC1, MAU2, ZC3H7B, UTY, FKBP5, TTC37, CDC23, |
| repeat | | CDC27, TTC17, STUB1, XAB2, SGTB, CTR9, IFIT3, RPAP3, FICD, KLC1, |
| | | IFIT5, EMC2, RANBP2, GTF3C3 |
| repeat: TPR 4 | 17 | TTC14, MAU2, UTY, TTC37, CDC23, NAA25, CLUH, CDC27, SGTB, TTC17, |
| | | CTR9, IFIT3, RPAP3, KLC1, IFIT5, VPS13A, GTF3C3 |
| repeat: TPR 3 | 23 | TTC31, TTC14, ZC3H7A, MAU2, ZC3H7B, UTY, FKBP5, TTC37, NAA25, |
| | | CDC23, CLUH, CDC27, TTC17, SGTB, STUB1, CTR9, IFIT3, RPAP3, KLC1, |
| | | IFIT5, VPS13A, EMC2, GTF3C3 |
| repeat: TPR 6 | 12 | RPAP3, IFIT3, UTY, KLC1, IFIT5, TTC37, CDC23, VPS13A, CDC27, TTC17, |
| | | GTF3C3, CTR9 |
| repeat: TPR 8 | 9 | IFIT3, UTY, IFIT5, TTC37, CDC23, VPS13A, CDC27, GTF3C3, CTR9 |
| repeat: TPR 7 | 10 | RPAP3, IFIT3, UTY, IFIT5, TTC37, CDC23, VPS13A, CDC27, GTF3C3, CTR9 |
| repeat: TPR 5 | 12 | RPAP3, IFIT3, UTY, KLC1, IFIT5, TTC37, CDC23, VPS13A, CDC27, TTC17, |
| | | GTF3C3, CTR9 |
| repeat: TPR 9 | 6 | TTC37, CDC23, VPS13A, CDC27, GTF3C3, CTR9 |
| repeat: TPR 10 | 4 | TTC37, VPS13A, GTF3C3, CTR9 |
| repeat: TPR 11 | 3 | TTC37, GTF3C3, CTR9 |
| Enrichment Score: | | |
| 1.0812045401748829 | | |
| GO: 0051287~NAD | 11 | CTBP1, ME2, IDH3G, PHGDH, IDH2, AHCYL1, GRHPR, GLYR1, HIBADH, |
| binding | | ALDH9A1, MDH1 |
| nucleotide phosphate-binding | 15 | CTBP1, ME2, SIRT6, SIRT7, HIBADH, ALDH3A2, SIRT2, DHRS7, IDH3G, |
| region: NAD | | PHGDH, OXNAD1, GLYR1, ALDH9A1, MDH1, HSD17B8 |
| binding site: NAD | 6 | CTBP1, ME2, PHGDH, GLYR1, HIBADH, MDH1 |
| Enrichment Score: | | |
| 1.0596664654758672 | | |
| GO: 0032481~positive regulation | 13 | IRAK1, POLR3F, POLR3H, ZC3HAV1, RELA, CREBBP, PTPN22, POLR3GL, |
| of type I interferon production | | POLR3C, POLR3E, STAT6, EP300, IRF3 |
| hsa00240: Pyrimidine | 21 | DCTD, POLR3F, POLR3H, NT5C3A, POLR1E, POLR1A, UPP1, DCK, |
| metabolism | | POLR3GL, POLR3C, PNP, POLR2B, POLR3E, NME3, POLE3, RRM1, |
| | | ENTPD6, TXNRD1, UCK1, TWISTNB, NT5C |
| hsa03020: RNA | 9 | POLR3F, POLR3H, POLR1E, POLR1A, POLR3GL, TWISTNB, POLR3C, |
| polymerase | | POLR2B, POLR3E |
| GO: 0006383~transcription from RNA | 9 | POLR3F, POLR3H, BRF1, TBP, IVNS1ABP, POLR3C, GTF3C1, POLR3E, |
| polymerase III promoter | | GTF3C3 |
| DNA-directed RNA | 8 | POLR3F, POLR3H, POLR1E, POLR1A, TWISTNB, POLR3C, POLR2B, |
| polymemse | | POLR3E |
| hsa04623: Cytosolic 12 DNA-sensing | | POLR3F, POLR3H, RELA, NFKBIB, RIPK1, PYCARD, POLR3GL, IRF3, |
| pathway | | POLR3C, CCL4, CHUK, POLR3E |
| GO: 0006359~regulation of transcription | 4 | POLR3F, BRF2, POLR3GL, POLR3C |
| from RNA polymerase III promoter | | |
| GO: 0003899~DNA-directed RNA | 8 | POLR3F, POLR3H, POLR1E, POLR1A, TWISTNB, POLR3C, POLR2B, |
| polymerase activity | | POLR3E |
| GO: 0001056~RNA polymerase III | 5 | POLR3F, POLR3H, POLR3GL, POLR3C, POLR3E |
| activity | | |
| GO: 0005666~DNA-directed RNA | 5 | POLR3F, POLR3H, POLR3GL, POLR3C, POLR3E |
| polymerase III complex | | |
| GO: 0001054~RNA | 3 | POLR1E, POLR1A, TWISTNB |
| polymerase I activity | | |
| GO: 0005736~DNA-directed RNA | 3 | POLR1E, POLR1A, TWISTNB |
| polymerase I complex | | |
| Enrichment Score: | | |
| 1.0231268518510328 | | |
| GO: 0051536-~iron-sulfur cluster | 7 | NFUl, TYW1, RSAD2, CDK5RAP1, CIAPIN1, NDUFS1, PPAT |
| binding | | |
| Iron-sulfur | 13 | GLRX5, TYW1, NDUFV2, IREB2, RSAD2, LIAS, UQCRFS1, CDK5RAP1, |
| | | CIAPIN1, NDUFS1, PPAT, GLRX2, REV3L |
| IPR007197: Radical SAM | 4 | TYW1, RSAD2, LIAS, CDK5RAP1 |
| 4Fe-4S | 8 | TYW1, IREB2, RSAD2, LIAS, CDK5RAP1, NDUFS1, PPAT, REV3L |
| GO: 0051539~4 iron, | 9 | NFU1, TYW1, IREB2, RSAD2, LIAS, CDK5RAP1, NDUFS1, PPAT, REV3L |
| 4 sulfur cluster binding | | |
| metal ion-binding site: Iron- | 3 | TYW1, RSAD2, CDK5RAP1 |
| sulfur (4Fe-4S-S-AdoMet) | | |
| SM00729: Elp3 | 3 | RSAD2, LIAS, CDK5RAP1 |
| IPR006638: Elongator protein | 3 | RSAD2, LIAS, CDK5RAP1 |
| 3/MiaB/NifB | | |
| metal ion-binding | 3 | IREB2, CDK5RAP1, PPAT |
| site: Iron-sulfur (4Fe-4S) | | |
| Enrichment Score: | | |
| 1.0174059112189755 | | |
| SM00800: uDENN | 5 | DENND5A, SBF1, MADD, DENND4B, DENND2D |
| SM00799: DENN | 5 | DENND5A, SBF1, MADD, DENND4B, DENND2D |
| SM00801: dDENN | 5 | DENND5A, SBF1, MADD, DENND4B, DENND2D |
| IPR005113: uDENN domain | 5 | DENND5A, SBF1, MADD, DENND4B, DENND2D |
| IPR001194: DENN domain | 5 | DENND5A, SBF1, MADD, DENND4B, DENND2D |
| IPR005112: dDENN domain | 5 | DENND5A, SBF1, MADD, DENND4B, DENND2D |
| domain.uDENN | 5 | DENND5A, SBF1, MADD, DENND4B, DENND2D |
| domain: DENN | 5 | DENND5A, SBF1, MADD, DENND4B, DENND2D |
| domain.dDENN | 5 | DENND5A, SBF1, MADD, DENND4B, DENND2D |
| GO: 0017112~Rabguanyl-nucleotide | 7 | RAB3GAP2, DENND5A, SBF1, MADD, TRAPPC4, DENND4B, DENND2D |
| exchange factor activity | | |
| Enrichment Score: | | |
| 1.013340358660417 | | |
| SM00809: Alpha_adaptinC2 | 4 | AP1G1, AP2A1, GGA1, GGA3 |
| IPR008152: Clathrin adaptor, | 4 | AP1G1, AP2A1, GGA1, GGA3 |
| alpha/beta/gamma-adaptin, appendage, | | |
| Ig-like subdomain | | |
| domain: GAE | 3 | AP1G1, GGA1, GGA3 |
| GO: 0030131~clathrin adaptor complex | 5 | AP3M2, AP1G1, AP3M1, GGA1, GGA3 |
| IPR008153: Clathrin adaptor, gamma- | 3 | AP1G1, GGA1, GGA3 |
| adaptin, appendage | | |
| IPR013041: Coatomer/clathrin adaptor | 4 | AP1G1, AP2A1, GGA1, GGA3 |
| appendage, Ig-like subdomain | | |
| Enrichment Score: | | |
| 0.997471155798655 | | |
| domain: CUE | 4 | N4BP2, ASCC2, AMFR, TAB3 |
| IPR003892: Ubiquitin system | 4 | N4BP2, ASCC2, AMFR, TAB3 |
| component Cue | | |
| SM00546: CUE | 3 | ASCC2, AMFR, TAB3 |
| Enrichment Score: | | |
| 0.9788942570619484 | | |
| GO: 0035267~NuA4 histone | 6 | ING3, ACTL6A, TRRAP, KAT5, MRGBP, BRD8 |
| acetyltransferase complex | | |
| GO: 0000812~Swr1 complex | 4 | ING3, TRRAP, KAT5, BRD8 |
| GO: 0043967~histone H4 acetylation | 7 | ING3, NCOA1, EP300, ACTL6A, USP22, TRRAP, BRD8 |
| GO: 0043968~histone H2A acetylation | 4 | ING3, ACTL6A, TRRAP, BRD8 |
| Enrichment Score: | | |
| 0.9757250476428545 | | |
| IPR005225: Small GTP-binding | 34 | RAB5B, RAB5C, ARF6, MTIF2, GFM2, ARL5A, KRAS, GFM1, RAC1, RALB, |
| protein domain | | RAB11B, SAR1B, RHOF, ARL2, RAP2C, EFTUD2, DRG1, DRG2, RAB33A, |
| | | RAB33B, ARL3, NRAS, RAB30, RAB18, RAB35, ARF4, RHOT1, RAB5A, |
| | | RHOT2, RIT1, ARL8B, ARL4C, NKIRAS2, ARL4A |
| GO: 0019003~GDP | 13 | RAP2C, RAB5B, RAB5C, RRAGC, ARL3, KRAS, RAB18, RAB35, RALB, |
| binding | | RAB11B, RAB5A, ARL8B, PRPS1 |
| IPR001806: Small | 19 | RAP2C, RAB5B, RAB5C, RAB33A, RAB33B, NRAS, RAB30, KRAS, RAB18, |
| GTPase superfamily | | RAB35, RAC1, RALB, RAB11B, RHOT1, RAB5A, RHOT2, RIT1, RHOF, |
| | | NKIRAS2 |
| short sequence motif: | 14 | RAP2C, RAB5B, RAB5C, NRAS, RAB30, KRAS, RAB18, RAB35, RAC1, |
| Effector region | | RAB11B, RAB5A, RALB, RHOF, NKIRAS2 |
| Prenylation | 21 | PHKA2, RAP2C, RAB5B, RAB5C, LMNB2, BROX, MIEN1, RAB33A, |
| | | RAB33B, NRAS, RAB30, KRAS, RAB18, PEX19, RAB35, RAC1, RALB, |
| | | RAB11B, RAB5A, YKT6, RHOF |
| lipid moiety-binding | 13 | RAP2C, RAB30, RAB18, RAB5B, RAB5C, RAB35, RAC1, RALB, RAB5A, |
| region: S-geranylgeranyl cysteine | | RAB11B, RHOF, RAB33A, RAB33B |
| propeptide: Removed | 13 | PSMB10, NRAS, CD55, RAP2C, KRAS, RAB30, RAB18, TPP1, CD59, RAC1, |
| in mature form | | RALB, RAB11B, RHOF |
| Enrichment Score: | | |
| 0.9749120956358032 | | |
| GO: 0005777~peroxisome | 20 | MVD, IDE, KIAA0430, MPV17, AKAP11, SZT2, PEX3, PMVK, ALDH3A2, |
| | | ACBD5, FAR1, MFF, FIS1, PNPLA8, GBF1, PEX19, PEX16, IDH2, GNPAT, |
| | | SCP2 |
| GO: 0016557~peroxisome | 3 | PEX19, PEX16, PEX3 |
| membrane biogenesis | | |
| Peroxisome biogenesis | 4 | PEX19, PEX2, PEX16, PEX3 |
| Peroxisome | 16 | ECI2, KIAA0430, SZT2, PEX3, PMVK, ACBD5, FAR1, MFF, FIS1, PEX19, |
| | | PEX2, PEX16, GNPAT, ACSL4, ACSL3, SCP2 |
| GO: 0005778~peroxisomal | 11 | FAR1, PNPLA8, PEX19, PEX2, PEX16, GNPAT, PEX3, ACSL4, ACSL3, |
| membmne | | ALDH3A2, ACBD5 |
| Zellweger syndrome | 4 | PEX19, PEX2, PEX16, PEX3 |
| GO: 0045046~protein import into | 3 | PEX19, PEX16, PEX3 |
| peroxisome membrane | | |
| GO: 0007031~peroxisome organization | 5 | PEX19, PEX2, PEX16, PEX3, SCP2 |
| hsa04146: Peroxisome | 14 | ECI2, MPV17, PEX3, PMVK, FAR1, PEX19, PEX2, PEX16, IDH2, GNPAT, |
| | | ACSL4, ACSL3, SCP2, ACSL5 |
| GO: 0005779~integral component | 4 | FIS1, PEX2, PEX16, PEX3 |
| of peroxisomal membrane | | |
| Peroxisome biogenesis disorder | 4 | PEX19, PEX2, PEX16, PEX3 |
| Enrichment Score: | | |
| 0.9705769247177995 | | |
| SM00571: DDT | 3 | BPTF, BAZ2B, BAZ2A |
| IPR018501: DDT domain superfamily | 3 | BPTF, BAZ2B, BAZ2A |
| domain: DDT | 3 | BPTF, BAZ2B, BAZ2A |
| Enrichment Score: | | |
| 0.9586769438708357 | | |
| GO: 0043015~gamma-tubulin binding | 7 | OFD1, TUBGCP5, CEP57, PDE4B, B9D2, MZT1, TUBGCP2 |
| GO: 0000923~equatorial | 3 | TUBGCP5, MZT1, TUBGCP2 |
| microtubule organizing center | | |
| GO: 0051415~interphase microtubule | 3 | TUBGCP5, MZT1, TUBGCP2 |
| nucleation by interphase microtubule | | |
| organizing center | | |
| Enrichment Score: | | |
| 0.9480710390828418 | | |
| compositionally biased region: | 4 | SMC5, SMC6, RAD50, SMC4 |
| Ala/Asp-rich (DA-box) | | |
| region of interest: Flexible hinge | 3 | SMC5, SMC6, SMC4 |
| IPR003395: RecF/RecN/SMC | 3 | SMC5, SMC6, SMC4 |
| Enrichment Score: | | |
| 0.9463520308175999 | | |
| SM00592: BRK | 4 | CHD9, CHD7, CHD6, SMARCA2 |
| IPR006576: BRK domain | 4 | CHD9, CHD7, CHD6, SMARCA2 |
| IPR000330: SNF2-related | 8 | CHD9, BTAF1, CHD7, CHD1L, INO80, CHD6, SMARCA2, TTF2 |
| IPR016197: Chromo domain-like | 7 | CHD9, CHD7, ARID4A, CBX1, KAT5, CHD6, CBX7 |
| SM00298: CHROMO | 7 | CHD9, CHD7, ARID4A, CBX1, KAT5, CHD6, CBX7 |
| IPR000953: Chromo domain/shadow | 7 | CHD9, CHD7, ARID4A, CBX1, KAT5, CHD6, CBX7 |
| domain.Chromo 1 | 4 | CHD9, CHD7, CBX1, CHD6 |
| domain.Chromo 2 | 3 | CHD9, CHD7, CHD6 |
| short sequence motif: DEAH box | 7 | CHD9, CHD7, CHD1L, DHX29, DHX16, CHD6, TTF2 |
| IPR023780: Chromo domain | 5 | CHD9, CHD7, CBX1, CHD6, CBX7 |
| Enrichment Score: | | |
| 0.9456837667749796 | | |
| domain: PCI | 7 | PSMD13, PSMD12, PCID2, COPS7A, PSMD3, COPS7B, COPS8 |
| Signalosome | 4 | COPS7A, COPS7B, COPS8, LESPA1 |
| GO: 0010388~cullin deneddylation | 3 | COPS7A, COPS7B, COPS8 |
| GO: 0008180~COP9 signalosome | 5 | WDR6, COPS7A, COPS7B, COPS8, LESPA1 |
| Enrichment Score: | | |
| 0.9432970300617087 | | |
| GO: 0003950~NAD + ADP- | 7 | ZC3HAV1, PARP12, PARP8, PARP11, SIRT6, PARP4, SIRT2 |
| ribosyltransferase activity | | |
| domain: PARP catalytic | 5 | ZC3HAV1, PARP12, PARP8, PARP11, PARP4 |
| IPR012317: Poly(AD P-ribose) | 5 | ZC3HAV1, PARP12, PARP8, PARP11, PARP4 |
| polymerase, catalytic domain | | |
| IPR004170: WWE domain | 4 | ZC3HAV1, PARP12, PARP11, RNF146 |
| Enrichment Score: | | |
| 0.916012864240317 | | |
| active site: Schiff-base intermediate | 3 | NEIL2, POLB, OGG1 |
| with DNA | | |
| GO: 0006284~base-excision repair | 8 | RPA1, XPA, MPG, NEIL2, USP47, SIRT6, POLB, OGG1 |
| hsa03410: Base excision repair | 7 | MPG, POLE3, NEIL2, MBD4, POLB, PARP4, OGG1 |
| Enrichment Score: | | |
| 0.9063741277695219 | | |
| Protein biosynthesis | 25 | FARS2, HBS1L, WARS2, MTIF2, TCEAL4, GFM2, EIF4EBP2, GFM1, EIF2B4, |
| | | EIF2B5, EIF4ENIF1, BRF1, EIF1B, MRRF, EIF2B1, EIF4G3, TRNAU1AP, |
| | | EIF4E, DHX29, TSFM, EEF1E1, FARSB, YARS2, EIF5A2, MTFMT |
| Initiation factor | 11 | EIF4ENIF1, EIF4G3, EIF4EBP2, EIF4E, BRF1, DHX29, ElF1B, MTIF2, |
| | | EIF2B1, EIF2B4, EIF2B5 |
| GO: 0003743~translation initiation | 12 | EIF4ENIF1, EIF4G3, EIF4EBP2, EIF4E, BRF1, DHX29, AG02, EIF1B, MTIF2, |
| factor activity | | EIF2B1, EIF2B4, EIF2B5 |
| GO: 0006413~translational initiation | 10 | RPL17, EIF4G3, EIF4E, DHX29, AGO2, EIF1B, EIF2B1, MTFMT, EIF2B4, |
| | | EIF2B5 |
| Enrichment Score: | | |
| 0.9057102063061746 | | |
| IPR014721: Ribosomal protein | 7 | GFM2, LONP1, MRPS9, MVD, GFM1, EFTUD2, TOP2B |
| S5 domain 2-type fold, subgroup | | |
| GO: 0032790~ribosome disassembly | 4 | GFM2, HBS1L, MTIF2, MRRF |
| SM00889: SM00889 | 3 | GFM2, GFM1, EFTUD2 |
| IPR005517: Translation elongation | 3 | GFM2, GFM1, EFTUD2 |
| factor EFG/EF2, domain IV | | |
| SM00838: SM00838 | 3 | GFM2, GFM1, EFTUD2 |
| IPR000640: Translation elongation | 3 | GFM2, GFM1, EFTUD2 |
| factor EFG, V domain | | |
| IPR009022: Elongation factor G, | 3 | GFM2, GFM1, EFTUD2 |
| III-V domain | | |
| IPR000795: Elongation factor, GTP- | 5 | GFM2, GFM1, EFTUD2, HBS1L, MTIF2 |
| binding domain | | |
| GO: 0003746~translation elongation | 7 | GFM2, TSFM, EEF1E1, GFM1, HBS1L, EIF5A2, TCEAL4 |
| factor activity | | |
| Elongation factor | 6 | GFM2, TSFM, GFM1, HBS1L, EIF5A2, TCEAL4 |
| 201 | | |
| IPR004161: Translation elongation | 4 | GFM2, GFM1, EFTUD2, HBS1L |
| factor EFTu/EF1A, domain 2 | | |
| IPR009000: Translation elongation/ | 5 | GFM2, GFM1, EFTUD2, HBS1L, MTIF2 |
| initiation factor/Ribosomal, beta-barrel | | |
| Enrichment Score: | | |
| 0.9035692912975634 | | |
| GO: 0070652~HAUS complex | 4 | HAUS3, HAUS6, HAUS2, HAUS1 |
| GO: 0051297-centrosome organization | 7 | ARL2, HAUS3, HAUS6, CEP120, BNIP2, HAUS2, HAUS1 |
| GO: 0051225-spindle assembly | 6 | HAUS3, HAUS6, CSNK1D, HAUS2, HAUS1, INO80 |
| Enrichment Score: | | |
| 0.9019097275135531 | | |
| GO: 0090630~activation | 19 | TBC1D10C, RABGAP1, RALGAPB, RABGAP1L, PIP5K1A, TBC1D22B, |
| of GTPase | | TBC1D15, RALGAPA1, NDEL1, TSC1, RCC2, SGSM2, TIAM1, SIPA1L1, |
| activity | | TBC1D13, TBC1D4, TBC1D1, AKT2, TBC1D9B |
| GO: 0031338~regulation of | 10 | TBC1D15, RABGAP1, TBC1D10C, SGSM2, TBC1D13, TBC1D4, RABGAP1L, |
| vesicle fusion | | TBC1D1, TBC1D22B, TBC1D9B |
| domain: Rab-GAP | 10 | TBC1D15, RABGAP1, TBC1D10C, SGSM2, TBC1D13, TBC1D4, RABGAP1L, |
| TBC | | TBC1D1, TBC1D22B, TBC1D9B |
| GO: 0017137~Rab | 22 | RAB3GAP2, DENND5A, TBC1D10C, RABGAP1, AP1G1, RABGAP1L, OPTN, |
| GTPase binding | | TBC1D22B, ANXA2, TBC1D15, UNC13D, PDE6D, SGSM2, AP3M1, |
| | | TBC1D13, RAC1, ACAP2, TBC1D4, SYTL3, EHD1, TBC1D1, TBC1D9B |
| GO: 0012505~endo | 19 | RABGAP1, TBC1D10C, TBC1D22B, BCL2L11, RTN3, TBC1D15, DOCK2, |
| membrane system | | CHMP1A, SGSM2, PGRMC1, PGRMC2, TBC1D13, TBC1D4, RNF167, |
| | | NSMAF, TBC1D1, NENF, CDC42EP3, TBC1D9B |
| SM00164: TBC | 10 | TBC1D15, RABGAP1, TBC1D10C, SGSM2, TBC1D13, TBC1D4, RABGAP1L, |
| | | TBC1D1, TBC1D22B, TBC1D9B |
| IPR000195: Rab-GTPase-TBC | 10 | TBC1D15, RABGAP1, TBC1D10C, SGSM2, TBC1D13, TBC1D4, RABGAP1L, |
| domain | | TBC1D1, TBC1D22B, TBC1D9B |
| GO: 1902017~regulation | 7 | TBC1D15, RABGAP1, TBC1D10C, TBC1D13, TBC1D1, TBC1D22B, |
| of cilium assembly | | TBC1D9B |
| domain: PID | 4 | ANKS1A, RABGAP1, RABGAP1L, TBC1D1 |
| SM00462: PTB | 5 | ANKS1A, RABGAP1, TBC1D4, RABGAP1L, TBC1D1 |
| IPR006020: Phospho | 5 | ANKS1A, RABGAP1, TBC1D4, RABGAP1L, TBC1D1 |
| tyrosine interaction domain | | |
| Enrichment Score: | | |
| 0.8869149577293349 | | |
| SM00717: SANT | 11 | TADA2A, DMTF1, MTA2, EZH1, MYB, TRERF1, NCOR2, RERE, TERF2, |
| | | ELMSAN1, TERF1 |
| IPR001005: SANT/ | 11 | TADA2A, DMTF1, MTA2, EZH1, MYB, TRERF1, NCOR2, RERE, TERF2, |
| Myb domain | | ELMSAN1, TERF1 |
| IPR017930: Myb domain | 4 | DMTF1, MYB, 1ERF2, TERF1 |
| GO: 0000118~histone deacetylase | 8 | TBL1XR1, MTA2, TBL1X, TRERF1, NCOR2, RERE, NRIP1, ELMSAN1 |
| complex | | |
| domain: SANT | 5 | TADA2A, MTA2, TRERF1, RERE, ELMSAN1 |
| IPR017884: SANT domain | 6 | TADA2A, MTA2, TRERF1, NCOR2, RERE, ELMSAN1 |
| domain: ELM2 | 4 | MTA2, TRERF1, RERE, ELMSAN1 |
| SM01189: SM01189 | 4 | MTA2, TRERF1, RERE, ELMSAN1 |
| IPR000949: ELM2 domain | 4 | MTA2, TRERF1, RERE, ELMSAN1 |
| domain: HTH myb-type | 3 | DMTF1, TERF2, TERF1 |
| DNA-binding | 4 | DMTF1, MYB, TERF2, TERF1 |
| region: H-T-H motif | | |
| IPR009057: Homeodomain-like | 20 | RABGAP1, POGZ, TADA2A, MTA2, ZHX1, CERS6, CERS4, ZEB1, TRERF1, |
| | | CASP8AP2, POGK, CERS2, DMTF1, HOPX, MYB, NCOR2, TERF2, RERE, |
| | | ELMSAN1, TERF1 |
| Enrichment Score: | | |
| 0.8828129877584019 | | |
| GO: 0070979~protein K11-finked | 8 | UBE2D4, UBE2A, RNF4, ANAPC4, UBE2W, CDC23, ANAPC10, CDC27 |
| ubiquitination | | |
| GO: 0051439~regulation of ubiquitin- | 6 | ANAPC4, CDC23, ANAPC10, CDC27, CDK2, UBE2E1 |
| protein ligase activity involved in | | |
| mitotic cell cycle | | |
| GO: 0005680~anaphase-promoting | 5 | CACUL1, ANAPC4, CDC23, ANAPC10, CDC27 |
| complex | | |
| GO: 0030071~regulation of mitotic | 3 | ANAPC4, CDC23, ANAPC10 |
| metaphase/anaphase transition | | |
| Enrichment Score: | | |
| 0.8760120979730552 | | |
| domain: Cytochrome | 5 | PGRMC1, PGRMC2, CYB5A, HERC2, NENF |
| b5 heme-binding | | |
| SM01117: SM01117 | 5 | PGRMC1, PGRMC2, CYB5A, HERC2, NENF |
| IPR001199: Cytochrome b5-like | 5 | PGRMC1, PGRMC2, CYB5A, HERC2, NENF |
| heme/steroid binding domain | | |
| GO: 0020037~heme binding | 5 | PGRMC1, SDHC, PGRMC2, CYB5A, JAK2 |
| Enrichment Score: | | |
| 0.8696720280102501 | | |
| domain: EH | 3 | SYNRG, EHD1, EHD4 |
| SM00027: EH | 4 | SYNRG, REPS1, EHD1, EHD4 |
| IPR000261: EPS15 homology (EH) | 4 | SYNRG, REPS1, EHD1, EHD4 |
| domain: EF-hand | 7 | GNPTAB, REPS1, STIM1, EHD1, ZZEF1, EHD4, TBC1D9B |
| Enrichment Score: | | |
| 0.859210233084765 | | |
| GO: 0045862~positive | 6 | EP300, CASP8, FADD, BAD, FBXW11, CLN6 |
| regulation of proteolysis | | |
| 46.P13K_PTEN | 6 | TNFRSF1A, CASP7, BCL2, CASP8, FADD, BAD |
| GO: 0097202~activation of cysteine- | 4 | CASP8, PYCARD, FADD, BAD |
| type endopeptidase activity | | |
| 86.Apoptosis_Nematode&_Vert | 4 | BCL2, CASP8, FADD, BAD |
| Enrichment Score: | | |
| 0.8578437407621964 | | |
| IPR020850: GTPase | 4 | DNM3, CREBZF, MX1, MX2 |
| effector domain, GED | | |
| IPR022812: Dynamin | 5 | DNM3, MX1, EHD1, MX2, EHD4 |
| SM00302: GED | 3 | DNM3, MX1, MX2 |
| IPR019762: Dynamin, GTPase region, | 3 | DNM3, MX1, MX2 |
| conserved site | | |
| IPR000375: Dynamin central domain | 3 | DNM3, MX1, MX2 |
| domain.GED | 3 | DNM3, MX1, MX2 |
| SM00053: DYNc | 3 | DNM3, MX1, MX2 |
| IPR003130: Dynamin | 3 | DNM3, MX1, MX2 |
| GTPase effector | | |
| IPR001401: Dynamin, | 3 | DNM3, MX1, MX2 |
| GTPase domain | | |
| Enrichment Score: | | |
| 0.85187387805759 | | |
| GO: 0000422~mitophagy | 11 | ATG2B, GABARAPL2, FIS1, ATG4B, RB1CC1, BNIP3, WIPI2, PPARGC1A, |
| | | WDR45B, WDR45, MARK2 |
| GO: 0034045~pre-autophagosomal | 5 | ATG2B, RB1CC1, WIPI2, WDR45B, WDR45 |
| structure membrane | | |
| GO: 0044804~nucleophagy | 5 | ATG2B, ATG4B, WIPI2, WDR45B, WDR45 |
| GO: 0034497~protein localization to | 4 | STX17, WIPI2, WDR45B, WDR45 |
| pre-autophagosomal structure | | |
| GO: 0080025~phosphatidylinositol-3,5- | 5 | GBF1, COMMD1, WIPI2, WDR45B, WDR45 |
| bisphosphate binding | | |
| GO: 0000045~autophagosome assembly | 7 | ATG2B, GABARAPL2, ATG4B, RB1CC1, WIPI2, WDR45B, WDR45 |
| GO: 0006497~protein lipidation | 3 | WIPI2, WDR45B, WDR45 |
| GO: 0032266~phosphatidylinosito1-3- | 5 | SNX19, WIPI2, SNX13, WDR45B, WDR45 |
| phosphate binding | | |
| Enrichment Score: | | |
| 0.842501378699483 | | |
| GO: 0061158~3′-UTR-mediated | 5 | ZFP36, ZFP36L2, KHSRP, QKI, ZC3H12D |
| mRNA destabilization | | |
| GO: 0017091~AU- | 5 | ZFP36, ZFP36L2, EXOSC7, TIA1, ELAVL1 |
| rich element binding | | |
| GO: 0035925~mRNA | 4 | ZFP36, ZFP36L2, KHSRP, ELAVL1 |
| 3′-UTR AU-rich region binding | | |
| GO: 0006402~mRNA | 5 | DI53, ZFP36, ZFP36L2, DCP2, KHSRP |
| catabolic process | | |
| GO: 0003730~mRNA 3′-UTR | 7 | ZFP36, ZFP36L2, TARDBP, FMR1, KHSRP, PUM1, ELAVL1 |
| binding | | |
| Enrichment Score: | | |
| 0.8299126897208382 | | |
| IPR000980: SH2 | 21 | VAV3, SOCS3, SOCS1, CBL, VAV1, STAT6, NCK2, SH2D3C, SH2D2A, |
| domain | | CBLB, SH2D3A, CRKL, RINL, ZAP70, JAK2, INPP5D, GRAP2, ABL2, |
| | | RASA1, PIK3R1, MATK |
| SM00252: SH2 | 18 | VAV3, SOCS3, SOCS1, VAV1, STAT6, NCK2, SH2D3C, SH2D2A, SH2D3A, |
| | | CRKL, ZAP70, JAK2, INPP5D, GRAP2, ABL2, RASA1, PIK3R1, MATK |
| SH2 domain | 18 | VAV3, SOCS3, SOCS1, VAV1, STAT6, NCK2, SH2D3C, SH2D2A, SH2D3A, |
| | | CRKL, ZAP70, JAK2, INPP5D, GRAP2, ABL2, RASA1, PIK3R1, MATK |
| GO: 0005070~SH3/SH2 | 10 | SH2D2A, NCK2, SH2D3C, SH3BGRL, VAV3, CRKL, SH2D3A, LASP1, |
| adaptor activity | | STAM, GRAP2 |
| domain.SH2 | 14 | VAV3, SOCS3, SOCS1, VAV1, STAT6, SH2D3C, SH2D2A, NCK2, SH2D3A, |
| | | CRKL, INPP5D, GRAP2, ABL2, MATK |
| domain SH3 1 | 5 | NCK2, VAV3, CRKL, GRAP2, VAV1 |
| domain.SH3 2 | 5 | NCK2, VAV3, CRKL, GRAP2, VAV1 |
| Enrichment Score: | | |
| 0.8204630664582663 | | |
| GO: 0050072~m7G(5′)pppN | 5 | NUDT1, NUDT4, DCP2, NUDT5, NUDT16L1 |
| diphosphatase activity | | |
| domain: Nudix hydrolase | 6 | NUDT1, NUDT4, DCP2, NUDT9, NUDT22, NUDT5 |
| IPR020084: NUDIX | 4 | NUDT1, NUDT4, DCP2, NUDT5 |
| hydrolase, conserved site | | |
| IPR000086: NUDIX hydrolase domain | 6 | NUDT1, NUDT4, DCP2, NUDT9, NUDT22, NUDT5 |
| GO: 0034656~nucleobase-containing | 3 | NUDT1, NUDT9, NUDT5 |
| small molecule catabolic process | | |
| IPR015797: NUDIX | 6 | NUDT1, NUDT4, DCP2, NUDT9, NUDT5, NUDT16L1 |
| hydrolase domain-like | | |
| short sequence motif: Nudix box | 5 | NUDT1, NUDT4, DCP2, NUDT9, NUDT5 |
| GO: 0030515-snoRNA binding | 5 | NUDT1, NUDT4, NUDT5, NUDT16L1, PWP2 |
| Enrichment Score: | | |
| 0.8167271376205175 | | |
| zinc finger region: MYM-type 2 | 3 | ZMYM2, ZMYM4, ZMYM5 |
| zinc finger region: MYM-type 3 | 3 | ZMYM2, ZMYM4, ZMYM5 |
| zinc finger region: MYM-type 1 | 3 | ZMYM2, ZMYM4, ZMYM5 |
| zinc finger region: MYM-type 4 | 3 | ZMYM2, ZMYM4, ZMYM5 |
| IPR010507: Zinc finger, MYM-type | 3 | ZMYM2, ZMYM4, ZMYM5 |
| SM00746: TRASH | 3 | ZMYM2, ZMYM4, ZMYM5 |
| IPR011017: TRASH domain | 3 | ZMYM2, ZMYM4, ZMYM5 |
| 0.8116396013515808 | | |
| repeat: HAT 6 | 4 | CRNKL1, SFIl, SART3, XAB2 |
| repeat: HAT 5 | 4 | CRNKL1, SFIl, SART3, XAB2 |
| repeat: HAT 8 | 3 | CRNKL1, SART3, XAB2 |
| repeat: HAT 4 | 4 | CRNKL1, SFIl, SART3, XAB2 |
| repeat: HAT 2 | 4 | CRNKL1, SFIl, SART3, XAB2 |
| repeat: HAT 1 | 4 | CRNKL1, SFIl, SART3, XAB2 |
| repeat: HAT 3 | 4 | CRNKL1, SFIl, SART3, XAB2 |
| repeat: HAT 7 | 3 | CRNKL1, SART3, XAB2 |
| SM00386: HAT | 3 | CRNKL1, SART3, XAB2 |
| IPR003107: RNA-processing protein, | 3 | CRNKL1, SART3, XAB2 |
| HAT helix | | |
| Enrichment Score: | | |
| 0.8053738526498081 | | |
| GO: 0005655~nucleolar ribonuclease | 4 | RPP38, POP4, POPS, POP7 |
| P complex | | |
| GO: 0004526~ribonuclease P activity | 4 | RPP38, POP4, POPS, POP7 |
| GO: 0001682-tRNA 5′-leader | 4 | RPP38, POP4, POPS, POP7 |
| removal | | |
| hsa03008: Ribosome biogenesis in | 10 | RPP38, REX01, GNL3L, NATIO, POP4, POPS, SPATA5, RBM28, POP7, PWP2 |
| eukaryotes | | |
| Enrichment Score: | | |
| 0.8035933138094512 | | |
| GO: 0004722~protein serine/threonine | 12 | MTMR14, RPAP2, PPP2CB, PPP3CB, DUSP23, PPM1A, MTMR6, PPP1R15B, |
| phosphatase activity | | UBL CP1, P IEN, PPP1CB, PPP2R2D |
| Protein phosphatase | 20 | PTPN7, PTPRE, PTPRA, STYX, DUSP23, PPM1A, DUSP22, PTPN22, |
| | | DUSP12, PPP1CB, PTEN, DUSP4, DUSP28, PGP, RPAP2, PPP2CB, DUSP16, |
| | | PPP3CB, CTD SP1, UBL CP1 |
| GO: 0006470~protein | 16 | PTPN7, PTPRE, STYX, PPM1A, DUSP22, PTPN22, PPP1CB, PIEN, SBF1, |
| dephosphmylation | | BCL2, PPP2CB, PPP3CB, CTDSP1, UBLCP1, MTMR6, FBXW11 |
| Enrichment Score: | | |
| 0.8010857129206969 | | |
| GO: 0034450~ubiquitin-ubiquitin | 5 | PRPF19, PELI1, UBE4A, AMFR, STUB1 |
| ligase activity | | |
| SM00504: Ubox | 3 | PRPF19, UBE4A, STUB1 |
| IPR003613: U box domain | 3 | PRPF19, UBE4A, STUB1 |
| Enrichment Score: | | |
| 0.7901368844841442 | | |
| GO: 0005868-cytoplasmic | 8 | DYNC1LI2, DYNLT3, SNX4, DYNLT1, DYNC1H1, DYNLRB1, BCL2L11, |
| dynein complex | | DYNC1I2 |
| Dynein | 6 | DYNC1LI2, DYNLT3, DYNLT1, DYNC1H1, DYNLRB1, DYNC1I2 |
| GO: 0007018~microtubule- | 9 | KIF3B, DYNC1LI2, SNX29, AP2A1, KLC1, DYNC1H1, DYNLRB1, DYNC1I2, |
| based movement | | ACTR10 |
| GO: 0003777~microtubule | 7 | KIF3B, DYNC1LI2, SNX29, KLC1, DYNC1H1, DYNLRB1, DYNC1I2 |
| motor activity | | |
| Motor protein | 11 | DNM3, KIF3B, DYNC1LI2, KLC1, MY01G, DYNLT3, MY09B, DYNLT1, |
| | | DYNC1H1, DYNLRB1, DYNC1I2 |
| Enrichment Score: | | |
| 0.7810728163809908 | | |
| domain: Leucine-zipper | 23 | E2F3, BACH2, E2F4, CREBZF, CREB1, TSN, MED13L, FOXP3, SREBF2, |
| | | ATF6, ATF5, FOS, TSC22D3, TCF20, JUN, MLLT10, NFE2L2, MLLT6, MYB, |
| | | NFE2L3, TCF3, CHUK, API5 |
| IPR004827: Basic- | 10 | CREBRF, ATF6, ATF5, FOS, BACH2, CREBZF, JUN, CREB1, NFE2L2, |
| leucine zipper domain | | NFE2L3 |
| IPR008917: Eukaryotic | 4 | BACH2, JUN, NFE2L2, NFE2L3 |
| transcription factor, | | |
| Skn-l-like, DNA-binding | | |
| SM00338: BRLZ | 8 | ATF6, ATF5, FOS, BACH2, JUN, CREB1, NFE2L2, NFE2L3 |
| IPR004826: Basic leucine zipper | 3 | BACH2, NFE2L2, NFE2L3 |
| domain, Maf-type | | |
| DNA-binding | 16 | BACH2, CREBZF, CREB1, MXI1, MXD4, SREBF2, ATF6, ATF5, FOS, |
| region: Basic motif | | NCOA1, NCOA2, HES4, JUN, NFE2L2, NFE2L3, TCF3 |
| Enrichment Score: | | |
| 0.7790223053007594 | | |
| IPR018503: Tetraspanin, | 6 | CD37, TSPAN5, CD81, CD63, CD151, TSPAN17 |
| conserved site | | |
| PIR5F002419: tetraspanin | 7 | CD37, TSPAN31, TSPAN5, CD81, CD63, CD151, TSPAN17 |
| IPR000301: Tetraspanin | 7 | CD37, TSPAN31, TSPAN5, CD81, CD63, CD151, TSPAN17 |
| IPR018499: Tetraspanin/Peripherin | 7 | CD37, TSPAN31, TSPAN5, CD81, CD63, CD151, TSPAN17 |
| IPR008952: Tetraspanin, EC2 domain | 6 | CD37, TSPAN5, CD81, CD63, CD151, TSPAN17 |
| 73.Integrins_and_other_cell- | 4 | CD37, CD81, CD63, CD151 |
| surface_receptors | | |
| Enrichment Score: | | |
| 0.7789647933825644 | | |
| Steroid biosynthesis | 9 | HSD17B11, EBP, MSMO1, MVD, HINT2, HMGCS1, PRKAA1, PMVK, |
| | | HSD17B8 |
| Sterol biosynthesis | 6 | EBP, MSMO1, MVD, HMGCS1, PRKAA1, PMVK |
| Cholesterol biosynthesis | 5 | EBP, MVD, HMGCS1, PRKAA1, PMVK |
| Cholesterol | 10 | SOAT1, EBP, APOL1, NPC2, INSIG2, MVD, HMGCS1, PRKAA1, PMVK, |
| metabolism | | SREBF2 |
| GO: 0006695~cholesterol biosynthetic | 8 | EBP, MSMO1, G6PD, INSIG2, MVD, HMGCS1, PRKAA1, PMVK |
| process | | |
| Sterol metabolism | 11 | SOAT1, EBP, APOL1, MSM01, NPC2, INSIG2, MVD, HMGCS1, PRKAA1, |
| | | PMVK, SREBF2 |
| hsa00900: Terpenoid backbone | 5 | NUS1, MVD, HMGCS1, PMVK, ACAT2 |
| biosynthesis | | |
| Steroid metabolism | 11 | SOAT1, EBP, APOL1, MSMO1, NPC2, INSIG2, MVD, HMGCS1, PRKAA1, |
| | | PMVK, SREBF2 |
| GO: 0008203~cholesterol metabolic | 9 | STARD3, SOAT1, APOL2, EBP, APOL1, NPC2, INSIG2, CLN6, SREBF2 |
| process | | |
| Enrichment Score: | | |
| 0.7599731845015745 | | |
| zinc finger region: UBR-type | 3 | UBR7, UBR2, FBXO11 |
| SM00396: ZnF_UBR1 | 3 | UBR7, UBR2, FBXO11 |
| IPR003126: Zinc finger, N-recognin | 3 | UBR7, UBR2, FBX011 |
| Enrichment Score: | | |
| 0.7582256458667048 | | |
| GO: 0008654~phospholipid biosynthetic | 11 | CDIPT, CRLS1, PGS1, DGKE, LPGAT1, SERINC1, HEXB, MBOAT1, |
| process | | PCYT1A, PIP5K1A, PTDSS1 |
| Phospholipid | 9 | CDIPT, CRLS1, PGS1, LPGAT1, SERINC1, MBOAT1, ABHD3, PCYT1A, |
| metabolism | | PTDSS1 |
| GO: 0016780~phosphotransferase | 3 | CDIPT, CRLS1, PGS1 |
| activity, for other substituted | | |
| phosphate groups | | |
| Phospholipid biosynthesis | 8 | CDIPT, CRLS1, PGS1, LPGAT1, SERINC1, MBOAT1, PCYT1A, PTDSS1 |
| GO: 0047144~2-acylglycerol-3- | 3 | CRLS1, LPGAT1, MBOAT1 |
| phosphate O-acyltransferase activity | | |
| hsa00564: Glycerophospholipid | 13 | PLD3, CDIPT, CRLS1, PGS1, DGKE, LPGAT1, MBOAT1, DGKZ, GNPAT, |
| metabolism | | DGKH, PCYT1A, PTDSS1, LPIN1 |
| GO: 0003841~1-acylglycerol-3- | 3 | CRL Sl, LPGAT1, MBOAT1 |
| phosphate O-acyltransferase activity | | |
| Enrichment Score: | | |
| 0.7529768935912621 | | |
| GO: 0000123~histone acetyltransferase | 7 | ING4, ELP2, EP300, KANSL1, WDR5, CREBBP, TRRAP |
| complex | | |
| GO: 0043984~histone H4-K16 | 6 | MSL2, ING4, KANSL1, KMT2A, MSL1, WDR5 |
| acetylation | | |
| GO: 0043981~histone H4-K5 | 3 | ING4, KANSL1, WDR5 |
| acetylation | | |
| GO: 0043982~histone H4-K8 acetylation | 3 | ING4, KANSL1, WDR5 |
| Enrichment Score: | | |
| 0.7505920508733883 | | |
| GO: 0000724~double-strand break | 14 | RAD51C, HUS1, SMC5, INO80, SMC6, RAD50, ATM, WDR48, RPA1, |
| repair via homologous recombination | | RECQL, NABP1, NSMCE1, RNF138, NSMCE2 |
| GO: 0000722~telomere maintenance | 7 | RPA1, RAD51C, RFC2, SMC5, NSMCE2, SMC6, RAD50 |
| via recombination | | |
| DNA recombination | 11 | RPA1, RAD51C, NSMCE1, SMC5, INO80, NSMCE2, SMC6, ACTL6A, |
| | | INO80D, INO80C, INO80B |
| Enrichment Score: | | |
| 0.7363395749707299 | | |
| GO: 0030422~production of siRNA | 3 | PRKRA, DICER1, MRPL44 |
| involved in RNA interference | | |
| GO: 0031054~pre-miRNA processing | 4 | PRKRA, DICER1, AGO2, MRPL44 |
| IPR014720: Double-stranded RNA- | 6 | CDKN2AIP, PRKRA, DICER1, STAU2, STAU1, MRPL44 |
| binding-like domain | | |
| Enrichment Score: | | |
| 0.7343641660048325 | | |
| repeat: HEAT 5 | 8 | BTAF1, EIF4G3, NIPBL, KIAA0368, CANDI, PSWIE4, TNPO2, TNPO1 |
| repeat: HEAT 3 | 10 | HEATR6, BTAF1, EIF4G3, NIPBL, KIAA0368, TBCD, CANDI, PSME4, |
| | | TNPO2, TNPO1 |
| repeat: HEAT 4 | 9 | HEATR6, BTAF1, EIF4G3, NIPBL, KIAA0368, CAND1, PSME4, TNPO2, |
| | | TNPO1 |
| repeat: HEAT 2 | 11 | HEATR6, BTAF1, EIF4G3, NIPBL, KIAA0368, TBCD, CAND1, PSME4, |
| | | TNPO2, UTP20, TNPO1 |
| repeat: HEAT 1 | 11 | HEATR6, BTAF1, EIF4G3, NIPBL, KIAA0368, TBCD, CAND1, PSME4, |
| | | TNPO2, UTP20, TNPO1 |
| repeat: HEAT 8 | 5 | BTAF1, KIAA0368, CAND1, TNPO2, TNPO1 |
| repeat: HEAT 6 | 6 | BTAF1, KIAA0368, CAND1, PSME4, TNPO2, TNPO1 |
| repeat: HEAT 7 | 5 | BTAF1, KIAA0368, CAND1, TNPO2, TNPO1 |
| repeat: HEAT 13 | 3 | KIAA0368, CAND1, TNPO2 |
| repeat: HEAT 12 | 3 | KIAA0368, CAND1, TNPO2 |
| repeat: HEAT 11 | 3 | KIAA0368, CAND1, TNPO2 |
| repeat: HEAT 10 | 3 | KIAA0368, CAND1, TNPO2 |
| repeat: HEAT 9 | 3 | KIAA0368, CAND1, TNPO2 |
| Enrichment Score: | | |
| 0.7306880194943396 | | |
| GO: 0070555~response to interleukin-1 | 8 | IRAK1, RELA, IGBP1, CREBBP, ANXA1, PRKCI, RIPK2, LGALS9 |
| GO: 0034134~toll-like receptor 2 | 3 | IRAK1, RIPK2, LGALS9 |
| signaling pathway | | |
| GO: 0034142~toll-like receptor 4 | 4 | IRAK1, LY96, RIPK2, LGALS9 |
| signaling pathway | | |
| Enrichment Score: | | |
| 0.7265386581976017 | | |
| domain: JmjC | 8 | KDM2A, UTY, JMJD6, JMJD8, KDM4C, JMJD1C, KDM5B, KDM5C |
| IPR003347: JmjC domain | 8 | KDM2A, UTY, JMJD6, JMJD8, KDM4C, JMJD1C, KDM5B, KDM5C |
| SM00558: JmjC | 7 | KDM2A, UTY, JMJD6, KDM4C, JMJD1C, KDM5B, KDM5C |
| GO: 0032452~histone | 6 | KDM2A, UTY, JMJD6, KDM4C, KDM5B, KDM5C |
| demethylase activity | | |
| domain: JmjN | 3 | KDM4C, KDM5B, KDM5C |
| Dioxygenase | 12 | ADI1, ALKBH7, KDM2A, UTY, JMJD6, ETHE1, KDM4C, EGLN1, JMJD1C, |
| | | KDM5B, ALKBH5, KDM5C |
| SM00545: JmjN | 3 | KDM4C, KDM5B, KDM5C |
| IPR003349: Transcription | 3 | KDM4C, KDM5B, KDM5C |
| factor jumonji, JmjN | | |
| GO: 0051213~dioxy genase activity | 4 | ALKBH7, UTY, KDM4C, JMJD1C |
| metal ion-binding site: Iron; catalytic | 4 | KDM2A, JMJD6, KDM4C, JMJD1C |
| Enrichment Score: | | |
| 0.7246856105034599 | | |
| zinc finger region: SP-RING-type | 3 | PIAS4, NSMCE2, PIAS1 |
| IPR004181: Zinc finger, MIZ-type | 3 | PIAS4, NSMCE2, PIAS1 |
| GO: 0019789~SUM O transferase | 4 | PIAS4, NSMCE2, PIAS1, RANBP2 |
| activity | | |
| Enrichment Score: | | |
| 0.7213724303242848 | | |
| GO: 0002223~stimulatory | 21 | PSMB10, RELA, CREBBP, ICAM3, RAF1, MALT1, PRKCD, TAB3, NRAS, |
| C-type lectin receptor | | PSMD13, PSMC5, EP300, KRAS, PSMD12, PSMC2, PSMD3, PRKACB, |
| signaling pathway | | PSMD5, PSME4, FBXW11, CHUK |
| GO: 0022624~proteasome accessory | 6 | PSMD13, PSMC5, PSMD12, PSMC2, PSMD3, PSMD5 |
| complex | | |
| GO: 0033209~tumor necrosis factor- | 20 | PSMB10, TRAF2, TNFRSF10A, TNFRSF1A, BAG4, TNFRSF9, PSMD13, |
| mediated signaling pathway | | PSMC5, PSMD12, TNFSF13B, PSMC2, RIPK1, PYCARD, PSMD3, JAK2, |
| | | PSMD5, PSME4, MAP3K14, CD27, TRAF3 |
| GO: 0051436~negative regulation of | 13 | PSMB10, ANAPC4, CDC23, ANAPC10, CDC27, CDK2, PSMD13, PSMC5, |
| ubiquitin-protein ligase activity | | PSMD12, PSMC2, PSMD3, PSMD5, UBE2E1 |
| involved in mitotic cell cycle | | |
| GO: 0031145~anaphase-promoting | 14 | PSMB10, ANAPC4, CDC23, ANAPC10, CDC27, CUL3, PSMD13, PSMC5, |
| complex-dependent catabolic process | | PSMD12, PSMC2, PSMD3, PSMD5, PSME4, UBE2E1 |
| GO: 0038061~NIK/NF-kappaB | 12 | PSMB10, PSMD13, PSMC5, PSMD12, PSMC2, UBA3, PSMD3, PSME4, |
| signaling | | PSMD5, MAP3K14, FBXW11, CHUK |
| GO: 0000502~proteasome complex | 11 | PSMB10, PSMD13, PSMC5, PSMD12, KIAA0368, ZFAND2A, PSMC2, |
| | | HSPB1, PSMD3, PSME4, PSMD5 |
| GO: 0051437~positive regulation of | 13 | PSMB10, ANAPC4, CDC23, ANAPC10, CDC27, PSMD13, PSMC5, PSMD12, |
| ubiquitin-protein ligase activity | | PSMC2, PSMD3, PSMD5, PSME4, UBE2E1 |
| involved in regulation of mitotic | | |
| cell cycle transition | | |
| GO: 0008541~proteasome regulatory | 3 | PSMD13, PSMD12, PSMD3 |
| particle, lid subcomplex | | |
| GO: 0031595~nuclear proteasome | 3 | PSMC5, PSMD12, PSMC2 |
| complex | | |
| GO: 0006521~regulation of cellular | 9 | PSMB10, PSMD13, PSMC5, PSMD12, PSMC2, PSMD3, AZIN1, PSME4, |
| amino acid metabolic process | | PSMD5 |
| hsa03050: Proteasome | 8 | PSMB10, PSMD13, PSMC5, PSMD12, PSMC2, IFNG, PSMD3, PSME4 |
| Proteasome | 8 | PSMB10, PSMD13, PSMC5, PSMD12, KIAA0368, PSMC2, PSMD3, PSME4 |
| GO: 0060071~Wnt | 13 | PSMB10, PSMD13, PSMC5, PSMD12, TIAM1, AP2A1, PSMC2, RAC1, |
| signaling pathway, | | PSMD3, SMURF2, PSME4, PSMD5, CLTC |
| planar cell polarity pathway | | |
| GO: 0008540~proteasome regulatory | 3 | PSMC5, PSMC2, PSMD5 |
| particle, base subcomplex | | |
| GO: 0090263~positive regulation of | 16 | PSMB10, RNF220, XIAP, PSMD13, PSMC5, PSMD12, CSNK1D, CSNK1E, |
| canonical Wnt signaling pathway | | PSMC2, ILK, PSMD3, SMURF2, PSMD5, PSME4, USP34, RNF146 |
| GO: 0002479~antigen processing and | 8 | PSMB10, PSMD13, PSMC5, PSMD12, PSMC2, PSMD3, PSME4, PSMD5 |
| presentation of exogenous peptide | | |
| antigen via MHC class I, TAP-dependent | | |
| GO: 0090090~negative regulation of | 17 | CSNK1A1, PSMB10, EGR1, RGS19, LATS1, CUL3, PSMD13, PSMC5, |
| canonical Wnt signaling pathway | | PSMD12, GSK3B, PSMC2, KIAA0922, PSMD3, PSMD5, PSME4, RAPGEF1, |
| | | APC |
| Enrichment Score: | | |
| 0.7193769298441833 | | |
| domain: UBX | 4 | UBXN2A, UBXN2B, FAF2, FAF1 |
| IPR001012: UBX | 4 | UBXN2A, UBXN2B, FAF2, FAF1 |
| SM00166: UBX | 3 | UBXN2A, UBXN2B, FAF1 |
| Enrichment Score: | | |
| 0.7013692853684937 | | |
| GO: 0004843~thiol-dependent ubiquitin- | 15 | STAMBP, OTUD5, USP3, USPS, USP4, BAP1, USP47, USP36, UCHL3, USP22, |
| specific protease activity | | USP34, USP16, USP15, USP42, VCPIP1 |
| Thiol protease | 23 | CAPN7, OTUD5, USP40, USPL1, USP3, USPS, USP4, BAP1, CTSL, ATG4B, |
| | | CASP7, CASP8, USP47, USP36, CTSC, UCHL3, USP22, USP34, USP16, |
| | | USP24, USP15, USP42, VCPIP1 |
| IPR018200: Peptidase C19, ubiquitin | 12 | USP40, USP3, USPS, USP4, U5P47, U5P36, U5P22, USP16, U5P34, U5P24, |
| carboxyl-terminal hydrolase 2, | | U5P42, USP15 |
| conserved site | | |
| GO: 0016579~protein | 15 | STAMBP, OTUD5, USP40, USP3, USP5, USP4, BAP1, WDR48, UCHL3, |
| deubiquitination | | USP36, USP22, USP34, USP24, USP15, USP42 |
| IPR001394: Peptidase C19, ubiquitin | 12 | USP40, USP3, USP5, USP4, USP47, USP36, USP22, USP16, USP34, USP24, |
| carboxyl-terminal hydrolase 2 | | USP42, USP15 |
| GO: 0036459~thiol-dependent | 9 | USP40, USP3, USP4, USP36, USP22, USP34, USP24, USP42, USP15 |
| ubiquitinyl hydrolase activity | | |
| Enrichment Score: | | |
| 0.694228209931004 | | |
| Glucose metabolism | 5 | G6PD, PDK3, PGM1, DCXR, AKT2 |
| Carbohydrate | 14 | PHKA2, GNPDA2, PDK3, PPP1CB, GALM, PGP, G6PD, GSK3B, PGM1, |
| metabolism | | POFUT1, DCXR, YDJC, AKT2, PYGB |
| GO: 0006006~glucose metabolic | 11 | GALM, WDTC1, G6PD, GNPDA2, PDK3, PGM1, HECTD4, PRKAA1, OAS1, |
| process | | DCXR, AKT2 |
| Enrichment Score: | | |
| 0.6913577368544611 | | |
| zinc finger region: Phorbol- | 7 | DGKE, DGKZ, PRKCH, DGKH, PRKCD, PRKD3, PRKCB |
| ester/DAG-type 2 | | |
| zinc finger region: Phorbol- | 7 | DGKE, DGKZ, PRKCH, DGKH, PRKCD, PRKD3, PRKCB |
| ester/DAG-type 1 | | |
| SM00046: DAGKc | 4 | DGKE, DGKZ, DGKH, CERK |
| domain: DAGKc | 4 | DGKE, DGKZ, DGKH, CERK |
| IPR001206: Diacylglycerol | 4 | DGKE, DGKZ, DGKH, CERK |
| kinase, catalytic domain | | |
| IPR016064: ATP- | 4 | DGKE, DGKZ, DGKH, CERK |
| NAD kinase-like domain | | |
| SM00045: DAGKa | 3 | DGKE, DGKZ, DGKH |
| IPR000756: Diacylglycerol | 3 | DGKE, DGKZ, DGKH |
| kinase, accessory domain | | |
| hsa00561: Glycerolipid | 9 | DGKE, GLA, AKR1B1, MBOAT1, DGKZ, DGKH, LPIN1, ALDH3A2, |
| metabolism | | ALDH9A1 |
| GO: 0004143~diacyl glycerol kinase | 3 | DGKE, DGKZ, DGKH |
| activity | | |
| GO: 0046834~lipid phosphorylation | 3 | DGKE, DGKZ, CERK |
| GO: 0007205~protein kinase C- | 5 | DGKE, DGKZ, DGKH, PRKD3, IL2 |
| activating G-protein coupled | | |
| receptor signaling pathway | | |
| Enrichment Score: | | |
| 0.6727450347449508 | | |
| domain: Exonuclease | 4 | AEN, REXO1, ERI3, ISG20L2 |
| Exonuclease | 9 | DIS3, EXOSC10, RAD1, CNOT8, CNOT6L, AEN, REXO1, ERI3, ISG20L2 |
| SM00479: EXOIII | 4 | AEN, REXO1, ERI3, ISG20L2 |
| IPR013520: Exonuclease, | 4 | AEN, REXO1, ERI3, ISG20L2 |
| RNase T/DNA polymerase III | | |
| IPR012337: Ribonuclease H-like | 12 | EXOSC10, TEFM, CNOT8, KIAA1586, ZBED5, AEN, REXO1, AGO2, |
| domain | | RNASEH1, ERI3, ISG20L2, REV3L |
| GO: 0004527~exonuclease activity | 3 | AEN, REXO1, ERI3 |
| Enrichment Score: | | |
| 0.6697712403636995 | | |
| domain: BRCT 2 | 4 | MDC1, TP53BP1, MCPH1, BARD1 |
| domain: BRCT 1 | 4 | MDC1, TP53BP1, MCPH1, BARD1 |
| SM00292: BRCT | 4 | TP53BP1, MCPH1, PARP4, BARD1 |
| IPR001357: BRCT domain | 5 | MDC1, TP53BP1, MCPH1, PARP4, BARD1 |
| Enrichment Score: | | |
| 0.667787729765459 | | |
| GO: 0031146~SCF-dependent | 6 | FBXW7, FBXW5, FBXO6, FBXL5, FBXL15, FBXW11 |
| proteasomal ubiquitin-dependent | | |
| protein catabolic process | | |
| GO: 0019005~SCF | 9 | FBXW7, FBXW5, FBXO6, U5P47, FBXL5, FBXO25, FBXL15, FBXW11, |
| ubiquitin ligase complex | | SPOP |
| SM00256: FBOX | 7 | FBXW7, FBXW5, FBXO6, FBXL5, FBXW2, FBXW11, FBXO11 |
| domain: F-box | 11 | FBXW7, KDM2A, FBXW5, FBXO6, FBXL5, FBXO25, FBXW2, FBXO34, |
| | | FBXL15, FBXW11, FBXO11 |
| IPR001810: F-box | 11 | FBXW7, KDM2A, FBXW5, FBXO6, FBXL5, FBXO25, FBXW2, FBXO34, |
| domain, cyclin-like | | FBXL15, FBXW11, FBXO11 |
| Enrichment Score: | | |
| 0.6636972705479839 | | |
| GO: 1904354~negative regulation of | 3 | TERF2, ATM, RAD50 |
| telomere capping | | |
| Telomere | 8 | NSMCE1, SMC5, NSMCE2, SMC6, TINF2, TERF2, RAD50, TERF1 |
| GO: 0003691~double-stranded telomeric | 3 | TERF2, RAD50, TERF1 |
| DNA binding | | |
| GO: 0007004~telomere maintenance | 4 | TERF2, ATM, RAD50, TERF1 |
| via telomerase | | |
| GO: 0000723~telomere maintenance | 6 | RPA1, HSPA1A, TERF2, ATM, RAD50, TERF1 |
| Enrichment Score: | | |
| 0.6493097279002905 | | |
| domain: Deacetylase sirtuin-type | 3 | SIRT6, SIRT7, SIRT2 |
| IPR003000: Sirtuin family | 3 | SIRT6, SIRT7, SIRT2 |
| IPR026590: Sirtuin family, catalytic | 3 | SIRT6, SIRT7, SIRT2 |
| core domain | | |
| GO: 0070403~NAD + binding | 3 | SIRT6, SIRT7, SIRT2 |
| Enrichment Score: | | |
| 0.633804002670718 | | |
| domain: PI3K/PI4K | 5 | PIK3C2A, PI4K2B, TRRAP, PI4KB, ATM |
| IPR000403: Phosphatidylinositol | 5 | PIK3C2A, PI4K2B, TRRAP, PI4KB, ATM |
| 3-/4-kinase, catalytic domain | | |
| SM00146: PI3Kc | 4 | PIK3C2A, TRRAP, PI4KB, ATM |
| IPR018936: Phosphatidylinositol | 3 | PIK3C2A, PI4KB, ATM |
| 3/4-kinase, conserved site | | |
| Enrichment Score: | | |
| 0.6336349362349432 | | |
| SM00147: RasGEF | 7 | SH2D3C, SH2D3A, SOS1, RAPGEF6, RGL4, RAPGEF1, RALGDS |
| IPR023578: Ras guanine nucleotide | 7 | SH2D3C, SH2D3A, SOS1, RAPGEF6, RGL4, RAPGEF1, RALGDS |
| exchange factor, domain | | |
| IPR001895: Guanine-nucleotide | 7 | SH2D3C, SH2D3A, SOS1, RAPGEF6, RGL4, RAPGEF1, RALGDS |
| dissociation stimulator CDC25 | | |
| domain: Ras-GEF | 6 | SH2D3C, SOS1, RAPGEF6, RGL4, RAPGEF1, RALGDS |
| SM00229: RasGEFN | 4 | SOS1, RAPGEF6, RAPGEF1, RALGDS |
| domain.N-terminal Ras-GEF | 4 | SOS1, RAPGEF6, RAPGEF1, RALGDS |
| IPR000651: Ras-like guanine | 4 | SOS1, RAPGEF6, RAPGEF1, RALGDS |
| nucleotide exchange factor, | | |
| N-terminal | | |
| IPR019804: Ras guanine-nucleotide | 3 | SOS1, RAPGEF1, RALGDS |
| exchange factor, conserved site | | |
| Enrichment Score: | | |
| 0.6315415331478075 | | |
| GO: 0005086~ARF guanyl-nucleotide | 6 | NCK2, GBF1, ARF4, PSD4, CYTH2, ARFGEF2 |
| exchange factor activity | | |
| h_arapPathway: ADP-Ribosylation | 6 | COPA, GBF1, ASAP1, CYTH2, ARFGEF2, ARAP2 |
| Factor | | |
| IPR023394: SEC7-like, alpha | 4 | GBF1, PSD4, CYTH2, ARFGEF2 |
| orthogonal bundle | | |
| SM00222: Sec7 | 4 | GBF1, PSD4, CYTH2, ARFGEF2 |
| IPR000904: SEC7-like | 4 | GBF1, PSD4, CYTH2, ARFGEF2 |
| domain: SEC7 | 4 | GBF1, PSD4, CYTH2, ARFGEF2 |
| GO: 0032012~regulation of ARF | 4 | GBF1, PSD4, CYTH2, ARFGEF2 |
| protein signal transduction | | |
| Enrichment Score: | | |
| 0.6256109532042883 | | |
| repeat: MBT 3 | 3 | MBTD1, L3MBTL2, L3MBTL3 |
| repeat: MBT 2 | 3 | MBTD1, L3MBTL2, L3MBTL3 |
| repeat: MBT 1 | 3 | MBTD1, L3MBTL2, L3MBTL3 |
| SM00561: MBT | 3 | MBTD1, L3MBTL2, L3MBTL3 |
| IPR004092: Mbt repeat | 3 | MBTD1, L3MBTL2, L3MBTL3 |
| Enrichment Score: | | |
| 0.6246957617788174 | | |
| DNA-binding region: HMG box | 8 | TOX, TCF7, HMGXB4, BBX, HMGXB3, HMG20B, HBP1, TOX4 |
| SM00398: HMG | 9 | TOX, TCF7, HMGXB4, KMT2C, BBX, HMGXB3, HMG20B, HBP1, TOX4 |
| IPR009071: High mobility group | 9 | TOX, TCF7, HMGXB4, KMT2C, BBX, HMGXB3, HMG20B, HBP1, TOX4 |
| (HMG) box domain | | |
| Enrichment Score: | | |
| 0.6234949961410196 | | |
| GO: 0031588~nucleotide-activated | 5 | PRKAR2A, PRKAG2, PRKAB1, PRKAA1, SESN2 |
| protein kinase complex | | |
| hsa04710: Circadian rhythm | 8 | CSNK1D, CSNK1E, CREB1, PRKAG2, PRKAB1, PRKAA1, RORA, FBXW11 |
| GO: 0004679~AMP-activated protein | 3 | PRKAG2, PRKAB1, PRKAA1 |
| kinase activity | | |
| GO: 0006633~fatty | 9 | ELOVL1, MSMO1, PRKAG2, PRKAB1, FASN, PRKAA1, ACSL3, PCCB, |
| acid biosynthetic process | | HSD17B8 |
| h_chrebpPathway: C | 5 | PRKAR2A, PRKAG2, PRKAB1, PRKAA1, PRKACB |
| hREBP regulation by carbohydrates | | |
| and cAMP | | |
| h_leptinPathway: Reversal of Insulin | 3 | PRKAG2, PRKAB1, PRKAA1 |
| Resistance by Leptin | | |
| Fatty acid biosynthesis | 6 | ELOVL1, PRKAG2, PRKAB1, FASN, PRKAA1, HSD17B8 |
| hsa05410: Hypertrophic cardiomyopathy | 4 | PRKAG2, PRKAB1, PRKAA1, ITGB1 |
| (HCM) | | |
| Enrichment Score: | | |
| 0.6150311927361685 | | |
| Isomerase | 19 | ECI1, FUOM, ECI2, EBP, FKBP5, TMX3, PDIA4, PIN4, PMM2, PUS7, NKTR |
| | | PPIF, GALM, PPIG, PGM1, PPIL4, TOP2B, FKBP11, TRUB2 |
| Cyclosporin | 3 | PPIF, PPIG, NKTR |
| GO: 0016018~cyclosporin A binding | 3 | PPIF, PPIG, NKTR |
| Rotamase | 7 | PPIF, PPIG, FKBP5, PPIL4, PIN4, FKBP11, NKTR |
| GO: 0000413~protein peptidyl-prolyl | 8 | PPIF, PPIG, FKBP5, PPIL4, RANBP2, PIN4, FKBP11, NKTR |
| isomerization | | |
| domain: PPIase cyclophilin-type | 5 | PPIF, PPIG, PPIL4, RANBP2, NKTR |
| IPR002130: Cyclophilin-like peptidyl- | 5 | PPIF, PPIG, PPIL4, RANBP2, NKTR |
| prolyl cis-trans isomerase domain | | |
| GO: 0003755~peptidyl-prolyl cis-trans | 8 | PPIF, PPIG, FKBP5, PPIL4, RANBP2, PIN4, FKBP11, NKTR |
| isomerase activity | | |
| IPR020892: Cyclophilin-type peptidyl- | 4 | PPIF, PPIG, RANBP2, NKTR |
| prolyl cis-trans isomerase, | | |
| conserved site | | |
| IPR024936: Cyclophilin-type peptidyl- | 4 | PPIF, PPIG, PPIL4, NKTR |
| prolyl cis-trans isomerase | | |
| Enrichment Score: | | |
| 0.6145856221059713 | | |
| SM00666: PB1 | 4 | MAP3K3, NBR1, PRKCI, TFG |
| IPR000270: Phox/Bem1p | 4 | MAP3K3, NBR1, PRKCI, TFG |
| domain: OPR | 3 | MAP3K3, NBR1, PRKCI |
| Enrichment Score: | | |
| 0.6107555591477245 | | |
| GO: 0000159~protein phosphatase type | 6 | PPP2R5A, STRN3, PPP2CB, STRN, PPP2R5E, PPP2R2D |
| 2A complex | | |
| GO: 0008601~protein phosphatase type | 4 | PPP2R5A, IGBP1, PPP2R5E, PPP2R2D |
| 2A regulator activity | | |
| GO: 0034047~regulation of protein | 4 | PPP2R5A, IGBP1, PPP2R5E, PPP2R2D |
| phosphatase type 2A activity | | |
| Enrichment Score: | | |
| 0.6081273857570164 | | |
| domain: DRBM 3 | 3 | PRKRA, STAU2, STAU1 |
| IPR014720: Double-stranded RNA- | 6 | CDKN2AIP, PRKRA, DICER1, STAU2, STAU1, MRPL44 |
| binding-like domain | | |
| SM00358: DSRM | 4 | PRKRA, DICER1, STAU2, STAU1 |
| domain: DRBM 2 | 3 | PRKRA, STAU2, STAU1 |
| domain: DRBM 1 | 3 | PRKRA, STAU2, STAU1 |
| Enrichment Score: | | |
| 0.6034239543605878 | | |
| GO: 0070412~R-SMAD binding | 6 | FOS, TRIM33, JUN, PPM1A, SMAD3, LDLRAD4 |
| GO: 1902895~positive regulation of | 5 | FOS, RELA, JUN, SMAD3, SRF |
| pri-miRNA transcription from | | |
| RNA polymerase II promoter | | |
| GO: 0060395~SMAD protein signal | 6 | LNPEP, FOS, JUN, HIPK2, NUP93, SMAD3 |
| transduction | | |
| Enrichment Score: | | |
| 0.5981131911799106 | | |
| GO: 0008625~extrinsic apoptotic | 12 | TNFRSF10A, TNFRSF9, TNFRSF1A, MOAP1, CASP8AP2, BCL2, FADD, |
| signaling pathway via death domain | | BAD, DAXX, PIK3R1, DEDD2, CD27 |
| receptors | | |
| SM00208: TNFR | 4 | TNFRSF10A, TNFRSF9, TNFRSF1A, CD27 |
| repeat: TNFR-Cys 3 | 4 | TNFRSF10A, TNFRSF9, TNFRSF1A, CD27 |
| IPR001368: TNFR/NGFR | 4 | TNFRSF10A, TNFRSF9, TNFRSF1A, CD27 |
| cysteine-rich region | | |
| GO: 0005031~tumor necrosis factor- | 4 | TNFRSF10A, TNFRSF9, TNFRSF1A, CD27 |
| activated receptor activity | | |
| repeat: TNFR-Cys 2 | 4 | TNFRSF10A, TNFRSF9, TNFRSF1A, CD27 |
| repeat: TNFR-Cys 1 | 4 | TNFRSF10A, TNFRSF9, TNFRSF1A, CD27 |
| Enrichment Score: | | |
| 0.5972512359197256 | | |
| GO: 0006661~phosphatidylinositol | 12 | CDIPT, SH3YL1, MTMR14, PIK3C2A, INPP5D, PI4K2B, PIP5K1A, PI4KB, |
| biosynthetic process | | MTMR6, PIEN, PIK3R1, SACM1L |
| hsa04070: Phosphatidylinositol | 17 | CDIPT, PIK3C2A, PPIP5K2, DGKH, PI4K2B, PIP5K1A, PI4KB, PTEN, |
| signaling system | | TMEM55B, PRKCB, ITPR2, MTMR14, DGKE, DGKZ, INPP5D, MTMR6, |
| | | PIK3R1 |
| hsa00562: Inositol | 9 | CDIPT, MTMR14, PIK3C2A, INPP5D, PI4K2B, PIP5K1A, PI4KB, MTMR6, |
| phosphate metabolism | | PTEN |
| GO: 0046854~phosphatidylinositol | 6 | PIK3C2A, PI4K2B, PIP5K1A, PI4KB, VAV1, PIK3R1 |
| phosphorylation | | |
| Enrichment Score: | | |
| 0.5959966330149766 | | |
| IPR011249: Metalloenzyme, | 3 | UQCRC1, IDE, PITRM1 |
| LuxS/M16 peptidase-like | | |
| IPR011237: Peptidase M16 domain | 3 | UQCRC1, IDE, PITRM1 |
| IPR011765: Peptidase M16, N-terminal | 3 | UQCRC1, IDE, PITRM1 |
| IPR007863: Peptidase M16, C-terminal | 3 | UQCRC1, IDE, PITRM1 |
| domain | | |
| GO: 0004222~metalloendopeptidase | 6 | SPG7, UQCRC1, TRABD2A, IDE, PITRM1, NLN |
| activity | | |
| Enrichment Score: | | |
| 0.5695312810104746 | | |
| IPR003903: Ubiquitin | 6 | STAM2, ZFAND2B, HGS, DNAJB2, STAM, UIMC1 |
| interacting motif | | |
| SM00726: UIM | 4 | STAM2, ZFAND2B, DNAJB2, UIMC1 |
| repeat: UIM 2 | 3 | ZFAND2B, DNAJB2, UIMC1 |
| repeat: UIM 1 | 3 | ZFAND2B, DNAJB2, UIMC1 |
| Enrichment Score: | | |
| 0.5600728887088808 | | |
| Telomere | 8 | NSMCE1, SMC5, NSMCE2, SMC6, TINF2, TERF2, RAD50, TERF1 |
| GO: 0070187~telosome | 3 | TINF2, TERF2, TERF1 |
| GO: 0000783~nuclear telomere cap | 3 | TINF2, TERF2, TERF1 |
| complex | | |
| GO: 0016233~telomere capping | 4 | HIST4H4, TINF2, TERF2, TERF1 |
| GO: 0042162~telomeric DNA | 4 | SMG5, TINF2, TERF2, TERF1 |
| binding | | |
| Enrichment Score: | | |
| 0.5582412750967661 | | |
| 2.Cytokine_Receptors | 5 | MAPK1, SOS1, RAF1, VAV1, PIK3R1 |
| h_il2rbPathway: IL-2 | 12 | MAPK1, FOS, IL2RB, CRKL, SOCS3, BCL2, SOS1, SOCS1, CBL, RAF1, |
| Receptor Beta Chain | | BAD, PIK3R1 |
| in T cell Activation | | |
| h_ptenPathway: PTEN dependent | 7 | MAPK1, CDKN1B, SOS1, ILK, PTEN, ITGB1, PIK3R1 |
| cell cycle arrest and apoptosis | | |
| h_cdmacPathway: Cadmium induces | 6 | MAPK1, FOS, RELA, JUN, RAF1, PRKCB |
| DNA synthesis and proliferation | | |
| in macrophages | | |
| h_ghPathway: Growth Hormone | 8 | MAPK1, SOS1, SOCS1, RAF1, JAK2, SRF, PIK3R1, PRKCB |
| Signaling Pathway | | |
| 68.Mitogen_signaling_in_growth_con- | 4 | MAPK1, SOS1, MAP3K1, RAF1 |
| trol | | |
| h_igf1rPathway: Multiple antiapoptotic | 7 | MAPK1, PRKAR2A, SOS1, RAF1, PRKACB, BAD, PIK3R1 |
| pathways from IGF-1R signaling lead | | |
| to BAD phosphorylation | | |
| 82.TCR_and_Cap_or_SMAC | 5 | MAPK1, ZAP70, MAPK8, VAV1, WAS |
| h_ngfPathway: Nerve growth factor | 6 | FOS, JUN, SOS1, RAF1, MAPK8, PIK3R1 |
| pathway (NGF) | | |
| h_her2Pathway: Role of ERBB2 in | 6 | MAPK1, EP300, IL6ST, SOS1, RAF1, PIK3R1 |
| Signal Transduction and Oncology | | |
| 54.T-cell_anergy | 6 | MAPK1, SOS1, ZAP70, RAF1, MAPK8, IL2 |
| h_spryPathway: Sprouty regulation | 5 | MAPK1, SOS1, CBL, RAF1, RASA1 |
| of tyrosine kinase signals | | |
| h_cxcr4Pathway: CX CR4 Signaling | 6 | MAPK1, CXCR4, RELA, RAF1, PIK3R1, PRKCB |
| Pathway | | |
| 107.mRNA_translation- | 5 | MAPK1, EIF4E, RAF1, EIF2B 1, PIK3R1 |
| protein_synthesis | | |
| 63.LAT_couples_T-cell_receptor | 5 | MAPK1, SOS1, ZAP70, VAV1, PIK3R1 |
| h_ecmPathway: Erk and PI-3 Kinase | 5 | MAPK1, ROCK1, RAF1, ITGB1, PIK3R1 |
| Are Necessary for Collagen Binding | | |
| in Corneal Epithelia | | |
| 106.Glycogen_synthase-synthesis | 3 | MAPK1, RAF1, PIK3R1 |
| h_tffPathway: Trefoil Factors Initiate | 5 | MAPK1, SOS1, BAD, ITGB1, PIK3R1 |
| Mucosal Healing | | |
| h_sppaPathway: Aspirin Blocks Sig- | 4 | MAPK1, RAF1, ITGB1, PRKCB |
| naling Pathway Involved in Platelet | | |
| Activation | | |
| 105.Signaling_glucose_uptake | 3 | MAPK1, RAF1, PIK3R1 |
| h_erkPathway: Erk1/Erk2 Mapk | 5 | MAPK1, SOS1, MKNK2, RAF1, ITGB1 |
| Signaling pathway | | |
| h_ccr3Pathway: CCR3 signaling in | 4 | MAPK1, ROCK2, RAF1, PRKCB |
| Eosinophils | | |
| h_biopeptidesPathway: Bioactive | 6 | MAPK1, SOS1, RAF1, MAPK8, JAK2, PRKCB |
| Peptide Induced Signaling Pathway | | |
| 104.Insulin_signaling | 4 | MAPK1, SOS1, RAF1, PIK3R1 |
| GO: 0014066~regulation of | 5 | MAPK1, C3ORF58, PIP5K1A, VAV1, PIK3R1 |
| phosphatidylinositol 3-kinase signaling | | |
| Enrichment Score: | | |
| 0.5573531878002459 | | |
| zinc finger | 4 | ARIH2, CUL9, MIB2, RNF216 |
| region: RING-type 2 | | |
| IPR002867: Zinc | 4 | ARIH2, CUL9, RBCK1, RNF216 |
| finger, C6HC-type | | |
| zinc finger region: IBR-type | 3 | ARIH2, CUL9, RNF216 |
| SM00647: IBR | 3 | ARIH2, CUL9, RNF216 |
| Enrichment Score: | | |
| 0.5552661855962138 | | |
| h_crebPathway: Transcription factor | 8 | MAPK1, PRKAR2A, CREB1, SOS1, RAC1, PRKACB, PIK3R1, PRKCB |
| CREB and its extracellular signals | | |
| h_igf1rPathway: Multiple antiapoptotic | 7 | MAPK1, PRKAR2A, SOS1, RAF1, PRKACB, BAD, PIK3R1 |
| pathways from IGF-1R signaling | | |
| lead to BAD phosphorylation | | |
| h_badPathway: Regulation of | 6 | MAPK1, PRKAR2A, BCL2, PRKACB, BAD, PIK3R1 |
| BAD phosphorylation | | |
| h_mPRPathway: How Progesterone | 4 | ARPC1A, MAPK1, PRKAR2A, PRKACB |
| Initiates the Oocyte Maturation | | |
| Enrichment Score: | | |
| 0.5494432470362846 | | |
| SM00568: GRAM | 4 | SBF1, NSMAF, GRAMD1A, TBC1D9B |
| domain: GRAM | 4 | TSC22D3, SBF1, NSMAF, GRAMD1A |
| IPR004182: GRAM | 4 | SBF1, NSMAF, GRAMD1A, TBC1D9B |
| Enrichment Score: | | |
| 0.547117417296358 | | |
| GO: 1904885~beta-catenin destruction | 3 | CSNK1A1, GSK3B, APC |
| complex assembly | | |
| GO: 0030877~beta-catenin destruction | 4 | CSNK1A1, GSK3B, RGS19, APC |
| complex | | |
| h_wntPathway: WNT | 6 | CSNK1A1, CTBP1, CSNK1D, GSK3B, CREBBP, APC |
| Signaling Pathway | | |
| GO: 1904886~beta-catenin destruction | 3 | CSNK1A1, GSK3B, APC |
| complex disassembly | | |
| Enrichment Score: | | |
| 0.5364694676464031 | | |
| IPR013763: Cyclin-like | 9 | CCNT2, BRF1, BRF2, CCNH, CCNT1, CCNG1, CCNG2, GTF2B, CASD1 |
| GO: 0000079~regulation of cyclin- | 8 | CCNT2, CDKN1B, CCNT1, HERC5, CNPPD1, CDK7, CCNG1, PTEN |
| dependent protein serine/threonine | | |
| kinase activity | | |
| SM00385: CYCLIN | 7 | CCNT2, BRF1, CCNH, CCNT1, CCNG1, CCNG2, GTF2B |
| GO: 1901409~positive regulation of | 3 | CCNT2, CCNH, CCNT1 |
| phosphorylation of RNA polymerase II | | |
| C-terminal domain | | |
| Cyclin | 6 | CCNT2, CDKN1B, CCNH, CCNT1, CCNG1, CCNG2 |
| GO: 0016538~cyclin-dependent protein | 3 | CCNT2, CCNH, CCNT1 |
| serine/threonine kinase regulator | | |
| activity | | |
| IPR006671: Cyclin, N-terminal | 5 | CCNT2, CCNH, CCNT1, CCNG1, CCNG2 |
| GO: 0045737~positive regulation of | 4 | CCNT2, CDKN1B, CCNH, CCNT1 |
| cyclin-dependent protein | | |
| serine/threonine kinase activity | | |
| Enrichment Score: | | |
| 0.5349369033767776 | | |
| IPR016192: APOBEC/CMP deaminase, | 4 | DCTD, APOBEC3G, APOBEC3C, APOBEC3D |
| zinc-binding | | |
| GO: 0010529~negative regulation | 3 | APOBEC3G, APOBEC3C, APOBEC3D |
| of transposition | | |
| IPR016193: Cytidine 4 | | DCTD, APOBEC3G, APOBEC3C, APOBEC3D |
| deaminase-like | | |
| IPR002125: CMP/dCMP deaminase, | 4 | DCTD, APOBEC3G, APOBEC3C, APOBEC3D |
| zinc-binding | | |
| GO: 0016814~hydrolase activity, | 3 | APOBEC3G, APOBEC3C, APOBEC3D |
| acting on carbon-nitrogen (but not | | |
| peptide) bonds, in cyclic amidines | | |
| IPR013158: APOBE | 3 | APOBEC3G, APOBEC3C, APOBEC3D |
| C-like, N-terminal | | |
| Enrichment Score: | | |
| 0.5346599394830712 | | |
| SM00233: PH | 34 | OSBP, ASAP1, CYTH2, ARHGAP15, APBBlIP, TIAM1, SOS1, SNTB1, |
| | | RTKN2, IPCEF1, DOCK10, RASA1, RASA2, AKT2, DNM3, ARHGEF3, |
| | | OSBPL3, VAV3, ARHGEF1, ROCK1, ROCK2, PSD4, DGKH, VAV1, |
| | | PLEKHA3, PLEKHF2, SBF1, DEF6, ACAP1, ACAP2, OSBPL11, ARAP2, |
| | | PRKD3, PLEKHAl |
| IPR011993: Pleckstrin | 53 | OSBP, ARHGAP15, TIAM1, NECAP2, SNTB1, NECAP1, MSN, RANBP2, |
| homology-like | | NSMAF, DOCK10, AKT2, ARHGEF3, ANKS1A, ARHGEF1, ROCK1, ROCK2, |
| domain | | PSD4, WAS, MTMR12, SBF1, DEF6, ACAP1, FRMD4B, ACAP2, OSBPL11, |
| | | WASL, PRKD3, RABGAP1, LRBA, ASAP1, RABGAP1L, CYTH2, APBBlIP, |
| | | SOS1, RTKN2, TBC1D4, IPCEF1, TBC1D1, MTMR6, RASA1, RASA2, DNM3, |
| | | OSBPL3, VAV3, EVL, DGKH, VAV1, PLEKHA3, PLEKHF2, DCP1A, JAK2, |
| | | ARAP2, PLEKHAl |
| domain: PH | 30 | OSBP, ASAP1, CYTH2, ARHGAP15, APBBlIP, SOS1, RTKN2, IPCEF1, |
| | | DOCK10, RASA1, RASA2, AKT2, DNM3, ARHGEF3, OSBPL3, VAV3, |
| | | ARHGEF1, ROCK1, ROCK2, PSD4, DGKH, VAV1, PLEKHA3, PLEKHF2, |
| | | SBF1, DEF6, ACAP1, ACAP2, OSBPL11, PRKD3 |
| IPR001849: Pleckstrin | 34 | OSBP, ASAP1, CYTH2, ARHGAP15, APBBlIP, TIAM1, SOS1, SNTB1, |
| homology domain | | RTKN2, IPCEF1, DOCK10, RASA1, RASA2, AKT2, DNM3, ARHGEF3, |
| | | OSBPL3, VAV3, ARHGEF1, ROCK1, ROCK2, PSD4, DGKH, VAV1, |
| | | PLEKHA3, PLEKHF2, SBF1, DEF6, ACAP1, ACAP2, OSBPL11, ARAP2, |
| | | PRKD3, PLEKHAl |
| Enrichment Score: | | |
| 0.5310103373589384 | | |
| domain.Ubiquitin- | 9 | DDI2, HERPUD1, UHRF2, UBL4A, RBCK1, TMUB1, UBAC1, UBLCP1, |
| like | | HERPUD2 |
| IPR000626: Ubiquitin | 9 | DDI2, HERPUD1, UHRF2, UBL4A, SACS, RBCK1, TMUB1, UBLCP1, |
| | | HERPUD2 |
| SM00213: UBQ | 5 | HERPUD1, UHRF2, UBL4A, UBLCP1, HERPUD2 |
| Enrichment Score: | | |
| 0.5255766583646028 | | |
| GO: 0004386~helicase | 17 | BTAF1, DICER1, ANXA1, HELZ, CHD9, MOV10, CHD7, DDX23, DDX19A, |
| activity | | GTF2F2, DHX34, DDX50, DDX10, ERCC3, SMARCA2, DDX51, DDX42 |
| IPR011545: DNA/RNA helicase, | 12 | RECQL, DDX23, DHX29, DDX19A, DICER1, DHX34, DDX50, DHX16, |
| DEAD/DEAH box type, N-terminal | | SKIV2L2, DDX10, DDX51, DDX42 |
| IPR000629: RNA helicase, ATP- | 5 | DDX23, CETN2, DDX10, DDX51, DDX42 |
| dependent, DEAD-box, conserved site | | |
| GO: 0010501~RNA secondary | 7 | DDX23, DDX19A, DDX50, AGO2, DDX10, DDX51, DDX42 |
| structure unwinding | | |
| short sequence motif: Q motif | 6 | DDX23, DDX19A, DDX50, DDX10, DDX51, DDX42 |
| GO: 0004004~ATP-dependent RNA | 9 | DDX23, DHX29, DDX19A, DHX34, DDX50, DHX16, DDX10, DDX51, |
| helicase activity | | DDX42 |
| short sequence motif: DEAD box | 5 | DDX23, DDX19A, DDX10, DDX51, DDX42 |
| IPR014014: RNA helicase, DEAD-box | 5 | DDX23, DDX19A, DDX50, DDX10, DDX42 |
| type, Q motif | | |
| Enrichment Score: | | |
| 0.5235257150362578 | | |
| IPR003959: ATPase, | 11 | SPG7, LONP1, ATAD3A, PSMC5, RFC2, PSMC2, WRNIP1, ORC4, VPS4A, |
| AAA-type, core | | SPATA5, SPAST |
| IPR003960: ATPase, AAA-type, | 5 | PSMC5, PSMC2, VPS4A, SPATA5, SPAST |
| conserved site | | |
| SM00382: AAA | 15 | ABCF3, SPG7, WRNIP1, ABCB7, TOR2A, ATAD3A, LONP1, PSMC5, RFC2, |
| | | PSMC2, ORC4, VPS4A, DYNC1H1, SPATA5, SPAST |
| IPR003593: AAA + | 15 | ABCF3, SPG7, WRNIP1, ABCB7, TOR2A, ATAD3A, LONP1, PSMC5, RFC2, |
| ATPase domain | | PSMC2, ORC4, VPS4A, DYNC1H1, SPATA5, SPAST |
| Enrichment Score: | | |
| 0.5084367621409388 | | |
| GO: 0002230~positive regulation of | 20 | TMEM203, TNIK, CRNKL1, PML, PTPN22, MBD5, NUP93, APOBEC3G, |
| defense response to virus by host | | PEX3, FXR2, ANXA5, MRPS2, FAM13B, SIN3A, CD93, DNAAF2, RBM18, |
| | | PYCARD, ALKBH5, MDH1 |
| GO: 0098792~xenophagy | 16 | TMEM203, TNIK, CRNKL1, MBD5, NUP93, OPTN, PEX3, ANXA5, FXR2, |
| | | FAM13B, MRPS2, CD93, DNAAF2, RBM18, ALKBH5, MDH1 |
| GO: 0098779~mitophagy in response | 15 | TMEM203, CRNKL1, MBD5, NUP93, LARP1B, PEX3, ANXA5, FAM13B, |
| to mitochondrial depolarization | | MRPS2, CD93, DNAAF2, BLOC1S1, MEX3C, KRCC1, MDH1 |
| Enrichment Score: | | |
| 0.505808532604712 | | |
| domain: BAH | 3 | MTA2, ASH1L, RERE |
| SM00439: BAH | 3 | MTA2, ASH1L, RERE |
| IPR001025: Bromo adjacent | 3 | MTA2, ASH1L, RERE |
| homology (BAH) domain | | |
| Enrichment Score: | | |
| 0.4971762083345744 | | |
| GO: 0019706~protein-cysteine S- | 6 | GOLGA7, ZDHHC16, ZDHHC3, ZDHHC8, ZDHHC12, YKT6 |
| palmitoyltransfemse activity | | |
| zinc finger region: DHHC-type | 5 | ZDHHC16, ZDHHC3, KMT2C, ZDHHC8, ZDHHC12 |
| GO: 0016409~palmitoyltransferase | 4 | ZDHHC16, ZDHHC3, ZDHHC8, ZDHHC12 |
| activity | | |
| IPR001594: Zinc finger, DHHC-type, | 5 | ZDHHC16, ZDHHC3, KMT2C, ZDHHC8, ZDHHC12 |
| palmitoyltransferase | | |
| GO: 0018345~protein palmitoylation | 4 | ZDHHC16, ZDHHC3, ZDHHC8, ZDHHC12 |
| Enrichment Score: | | |
| 0.48722466637005296 | | |
| short sequence motif: TXY | 4 | MAPK1, MAPK6, MAPK13, MAPK8 |
| GO: 0004707~MAP | 4 | MAPK1, MAPK6, MAPK13, MAPK8 |
| kinase activity | | |
| IPR003527: Mitogen-activated protein | 3 | MAPK1, MAPK13, MAPK8 |
| (MAP) kinase, conserved site | | |
| hsa04723: Retrograde endocannabinoid | 7 | MAPK1, ADCY7, MAPK13, MAPK8, PRKACB, PRKCB, ITPR2 |
| signaling | | |
| Enrichment Score: | | |
| 0.4869638417073476 | | |
| h_cdmacPathway: Cadmium induces | 6 | MAPK1, FOS, RELA, JUN, RAF1, PRKCB |
| DNA synthesis and proliferation in | | |
| macrophages | | |
| GO: 1902895~positive | 5 | FOS, RELA, JUN, SMAD3, SRF |
| regulation of pri-miRNA transcription | | |
| from RNA polymerase II promoter | | |
| GO: 0035994~response to muscle | 4 | FOS, RELA, JUN, RAF1 |
| stretch | | |
| h_cardiacEGFPathway: Role of EGF | 4 | FOS, RELA, JUN, PRKCB |
| Receptor Transactivation by GPCRs | | |
| in Cardiac Hypertrophy | | |
| GO: 0051591~response to cAMP | 5 | FOS, BSG, RELA, JUN, CDK2 |
| Enrichment Score: | | |
| 0.48175618712662555 | | |
| GO: 0016791~phosphatase | 9 | DUSP4, DUSP28, DUSP16, DUSP23, CTDSP1, PTPN22, DUSP12, PPP1CB, |
| activity | | SACM1L |
| Protein phosphatase | 20 | PTPN7, PTPRE, PTPRA, STYX, DUSP23, PPM1A, DUSP22, PTPN22, |
| | | DUSP12, PPP1CB, PTEN, DUSP4, DUSP28, PGP, RPAP2, PPP2CB, DUSP16, |
| | | PPP3CB, CTDSP1, UBLCP1 |
| SM00195: DSPc | 7 | DUSP4, DUSP28, DUSP16, STYX, DUSP23, DUSP22, DUSP12 |
| IPR000340: Dual specificity | 8 | DUSP4, DUSP28, DUSP16, STYX, DUSP23, DUSP22, DUSP12, PTEN |
| phosphatase, catalytic domain | | |
| IPR020422: Dual specificity | 7 | DUSP4, DUSP28, DUSP16, STYX, DUSP23, DUSP22, DUSP12 |
| phosphatase, subgroup, catalytic | | |
| domain | | |
| GO: 0008138~protein tyrosine/serine/ | 7 | DUSP28, SBF1, STYX, DUSP23, DUSP22, DUSP12, PTEN |
| threonine phosphatase activity | | |
| IPR024950: Dual specificity | 6 | DUSP4, DUSP28, DUSP16, STYX, DUSP22, DUSP12 |
| phosphatase | | |
| active site: Phosphocysteine | 12 | PTPN7, DUSP4, DUSP28, PTPRE, PTPRA, DUSP16, DUSP23, DUSP22, |
| intermediate | | PTPN22, DUSP12, MTMR6, PEEN |
| domain: Tyrosine- | 9 | PTPN7, DUSP4, DUSP28, DUSP16, STYX, DUSP23, DUSP22, PTPN22, |
| protein phosphatase | | DUSP12 |
| GO: 0035335~peptidyl-tyrosine | 14 | PTPN7, PTPRE, PTPRA, DUSP23, PTPN22, DUSP22, DUSP12, PTEN, DUSP4, |
| dephosphorylation | | MTMR14, PGP, DUSP28, DUSP16, MTMR6 |
| GO: 0004725~protein tyrosine | 14 | PTPN7, PTPRE, PTPRA, DUSP23, PTPN22, DUSP22, DUSP12, PTEN, DUSP4, |
| phosphatase activity | | MTMR14, PGP, DUSP28, DUSP16, MTMR6 |
| IPR000387: Protein-tyrosine/Dual | 11 | PTPN7, DUSP4, DUSP28, PTPRE, PTPRA, DUSP16, STYX, DUSP23, DUSP22, |
| specificity phosphatase | | PTPN22, DUSP12 |
| IPR016130: Protein-tyrosine | 10 | PTPN7, DUSP4, MTMR14, PTPRE, PTPRA, DUSP16, DUSP23, PTPN22, |
| phosphatase, active site | | MTMR6, PEEN |
| SM00404: PTPc_motif | 8 | PTPN7, DUSP4, PTPRE, PTPRA, DUSP23, PTPN22, MTMR6, PTEN |
| IPR003595: Protein-tyrosine | 8 | PTPN7, DUSP4, PTPRE, PTPRA, DUSP23, PTPN22, MTMR6, PTEN |
| phosphatase, catalytic | | |
| GO: 0000188~inactivation of MAPK | 4 | DUSP4, DUSP16, DUSP22, GPS2 |
| activity | | |
| SM00194: PTPc | 4 | PTPN7, PTPRE, PTPRA, PTPN22 |
| IPR000242: Protein-tyrosine | 4 | PTPN7, PTPRE, PTPRA, PTPN22 |
| phosphatase, receptor/non- | | |
| receptor type | | |
| Enrichment Score: | | |
| 0.480513926399985 | | |
| IPR001715: Calponin | 14 | PARVG, VAV3, ACTN4, CEP95, UTRN, IQGAP2, VAV1, FLNA, SYNE2, |
| homology domain | | CAMSAP1, MAPRE2, CNN2, MAPRE1, PLEC |
| domain.CH 2 | 6 | PARVG, SYNE2, ACTN4, UTRN, FLNA, PLEC |
| domain.CH 1 | 6 | PARVG, SYNE2, ACTN4, UTRN, FLNA, PLEC |
| domain.Actin-binding | 5 | SYNE2, ACTN4, UTRN, FLNA, PLEC |
| IPR001589: Actinin-type, actin-binding, | 5 | SYNE2, ACTN4, UTRN, FLNA, PLEC |
| conserved site | | |
| SM00033: CH | 10 | PARVG, VAV3, SYNE2, ACTN4, UTRN, IQGAP2, CNN2, VAV1, FLNA, |
| | | PLEC |
| repeat: Spectrin 4 | 4 | SYNE2, ACTN4, UTRN, PLEC |
| repeat: Spectrin 3 | 4 | SYNE2, ACTN4, UTRN, PLEC |
| domain: CH | 6 | VAV3, IQGAP2, MAPRE2, CNN2, MAPRE1, VAV1 |
| repeat: Spectrin 2 | 4 | SYNE2, ACTN4, UTRN, PLEC |
| repeat: Spectrin 1 | 4 | SYNE2, ACTN4, UTRN, PLEC |
| SM00150: SPEC | 4 | SYNE2, ACTN4, UTRN, PLEC |
| IPR018159: Spectrin/alpha-actinin | 4 | SYNE2, ACTN4, UTRN, PLEC |
| IPR002017: Spectrin repeat | 3 | SYNE2, ACTN4, UTRN |
| Enrichment Score: | | |
| 0.469203568351973 | | |
| hsa04720: Long-term | 14 | CREBBP, RAF1, PPP1CB, PRKCB, ITPR2, NRAS, MAPK1, RPS6KA3, EP300, |
| potentiation | | KRAS, CAMK4, ARAF, PPP3CB, PRKACB |
| hsa05223: Non-small | 12 | MAPK1, NRAS, E2F3, KRAS, RXRB, SOS1, ARAF, RAF1, BAD, PIK3R1, |
| cell lung cancer | | PRKCB, AKT2 |
| hsa05214: Glioma | 11 | MAPK1, NRAS, E2F3, KRAS, SOS1, ARAF, RAF1, PTEN, PIK3R1, PRKCB, |
| | | AKT2 |
| 65.Integrin_affinity_modulation | 3 | MAPK1, NRAS, KRAS |
| hsa04730: Long-term depression | 9 | GNA13, MAPK1, NRAS, KRAS, PPP2CB, ARAF, RAF1, PRKCB, ITPR2 |
| hsa05218: Melanoma | 10 | MAPK1, NRAS, E2F3, KRAS, ARAF, RAF1, BAD, PTEN, PIK3R1, AKT2 |
| hsa04540: Gap | 12 | MAPK1, NRAS, KRAS, CSNK1D, ADCY7, SOS1, RAF1, PRKACB, TUBA1A, |
| junction | | TUBA1C, PRKCB, ITPR2 |
| hsa04921: Oxytocin | 20 | ROCK1, ADCY7, ROCK2, PRKAG2, PRKAB1, RAF1, PPP1CB, PRKCB, |
| signaling pathway | | ITPR2, FOS, NRAS, MAPK1, KRAS, CAMK4, JUN, PPP3CB, PRKAA1, |
| | | PRKACB, NFATC2, PIK3R1 |
| hsa05219: Bladder cancer | 6 | MAPK1, NRAS, E2F3, KRAS, ARAF, RAF1 |
| hsa04725: Cholinergic | 14 | ADCY7, CREB1, PRKCB, ITPR2, NRAS, MAPK1, FOS, KRAS, CAMK4, |
| synapse | | BCL2, JAK2, PRKACB, PIK3R1, AKT2 |
| hsa04916: Melanogenesis | 12 | MAPK1, NRAS, TCF7, KRAS, EP300, ADCY7, CREB1, GSK3B, CREBBP, |
| | | RAF1, PRKACB, PRKCB |
| hsa04726: Serotonergic synapse | 8 | MAPK1, NRAS, KRAS, ARAF, RAF1, PRKACB, PRKCB, ITPR2 |
| hsa05034: Alcoholism | 12 | MAPK1, NRAS, HIST4H4, KRAS, CAMK4, CREB1, SOS1, ARAF, RAF1, |
| | | H3F3A, PKIA, PPP1CB |
| Enrichment Score: | | |
| 0.4584026793393616 | | |
| domain: DHR-2 | 3 | DOCK2, DOCK8, DOCK10 |
| domain: DHR-1 | 3 | DOCK2, DOCK8, DOCK10 |
| IPR027357: DHR-2 domain | 3 | DOCK2, DOCK8, DOCK10 |
| IPR027007: DHR-1 domain | 3 | DOCK2, DOCK8, DOCK10 |
| IPR026791: Dedicator of cytokinesis | 3 | DOCK2, DOCK8, DOCK10 |
| IPR010703: Dedicator | 3 | DOCK2, DOCK8, DOCK10 |
| of cytokinesis C-terminal | | |
| Enrichment Score: | | |
| 0.45728497291694326 | | |
| Nucleotidyltransferase | 13 | POLK, FICD, CMAS, POLE3, POLR1A, OAS1, POLB, PCYT1A, PAPD5, |
| | | OAS2, ZCCHC6, POLR2B, REV3L |
| DNA-directed DNA polymemse | 5 | POLK, POLE3, POLB, PAPD5, REV3L |
| GO: 0003887~DNA-directed DNA | 5 | POLK, POLE3, POLB, PAPD5, REV3L |
| polymerase activity | | |
| GO: 0071897~DNA | 3 | POLE3, POLB, PAPD5 |
| biosynthetic process | | |
| Enrichment Score: | | |
| 0.44605748185239036 | | |
| Signal transduction inhibitor | 9 | RGS1, SOCS3, GSK3B, SOCS1, RGS19, SNX13, LDLRAD4, RGS14, SEC14L1 |
| GO: 0001965~G-protein alpha- | 5 | NUCB1, RGS1, IGF2R, RGS19, RGS14 |
| subunit binding | | |
| IPR016137: Regulator of G protein | 6 | ARHGEF1, RGS1, RGS19, AKAP10, SNX13, RGS14 |
| signalling superfamily | | |
| SM00315: RGS | 5 | RGS1, RGS19, AKAP10, SNX13, RGS14 |
| domain: RGS | 4 | RGS1, RGS19, SNX13, RGS14 |
| IPR024066: Regulator of G-protein | 3 | RGS1, RGS19, RGS14 |
| signaling, domain 1 | | |
| Enrichment Score: | | |
| 0.4417241398856797 | | |
| GO: 0000729~DNA double-strand | 4 | KAT5, ATM, RAD50, BARD1 |
| break processing | | |
| GO: 0000732~strand displacement | 5 | RAD51C, KAT5, ATM, RAD50, BARD1 |
| GO: 0000731~DNA | 6 | RAD51C, WRNIP1, KAT5, ATM, RAD50, BARD1 |
| synthesis involved in DNA repair | | |
| GO: 0007131~reciprocal meiotic | 5 | RAD51C, MSH6, MSH2, ATM, RAD50 |
| recombination | | |
| Enrichment Score: | | |
| 0.4412701245717094 | | |
| zinc finger region: CCHC-type 3 | 3 | ZCCHC3, ZCCHC6, ZCCHC7 |
| zinc finger region: CCHC-type 2 | 3 | ZCCHC3, ZCCHC6, ZCCHC7 |
| zinc finger region: CCHC-type 1 | 3 | ZCCHC3, ZCCHC6, ZCCHC7 |
| SM00343: ZnF_C2HC | 4 | ZCCHC3, CPSF4, ZCCHC6, ZCCHC7 |
| IPR001878: Zinc finger, CCHC-type | 5 | ZCCHC3, ZCCHC10, CPSF4, ZCCHC6, ZCCHC7 |
| Enrichment Score: | | |
| 0.42704739477485376 | | |
| h_crebPathway: Transcription factor | 8 | MAPK1, PRKAR2A, CREB1, SOS1, RAC1, PRKACB, PIK3R1, PRKCB |
| CREB and its extracellular signals | | |
| h_agpcrPathway: Attenuation of | 3 | PRKAR2A, PRKACB, PRKCB |
| GPCR Signaling | | |
| h_nos1Pathway: Nitric Oxide Signaling | 4 | PRKAR2A, PPP3CB, PRKACB, PRKCB |
| Pathway | | |
| Enrichment Score: | | |
| 0.42570578914927665 | | |
| h_gpcrPathway: Signaling Pathway | 10 | FOS, PRKAR2A, RPS6KA3, JUN, CREB1, PPP3CB, RAF1, PRKACB, |
| from G-Protein Families | | NFATC2, PRKCB |
| h_dreamPathway: Repression of Pain | 5 | FOS, PRKAR2A, JUN, CREB1, PRKACB |
| Sensation by the Transcriptional | | |
| Regulator DREAM | | |
| hsa05031: Amphetamine addiction | 8 | FOS, CAMK4, JUN, CREB1, PPP3CB, PRKACB, PPP1CB, PRKCB |
| hsa05030: Cocaine addiction | 4 | RELA, JUN, CREB1, PRKACB |
| hsa04713: Circadian entrainment | 6 | MAPK1, FOS, ADCY7, CREB1, PRKACB, PRKCB |
| Enrichment Score: | | |
| 0.4211948096599511 | | |
| GO: 0000132~establishment of mitotic | 6 | NUMA1, NDE1, NDEL1, MCPH1, PAFAH1B1, DYNLT1 |
| spindle orientation | | |
| GO: 2000574~regulation | 3 | NDE1, NDEL1, PAFAH1B1 |
| of microtubule motor activity | | |
| GO: 0047496~vesicle transport along | 3 | NDE1, NDEL1, PAFAH1B1 |
| microtubule | | |
| GO: 0001764~neuron migration | 9 | NDE1, NDEL1, CXCR4, CCR4, GATA3, PAFAH1B1, TOP2B, SRF, MARK2 |
| GO: 0005871~kinesin complex | 4 | NDE1, NDEL1, KLC1, PAFAH1B1 |
| Enrichment Score: | | |
| 0.4188790271143383 | | |
| domain: Ras-associating | 6 | RASSF3, RAPGEF6, MYO9B, APBB1IP, ARAP2, RALGDS |
| SM00314: RA | 5 | RASSF3, RAPGEF6, MYO9B, APBB1IP, RALGDS |
| IPR000159: Ras-association | 6 | RASSF3, RAPGEF6, MYO9B, APBB1IP, ARAP2, RALGDS |
| Enrichment Score: | | |
| 0.41763318415020556 | | |
| GO: 0030148~sphingolipid | 10 | ELOVL1, SPTLC2, CSNK1G2, VAPB, CERS2, CERS6, SPTSSA, KDSR, |
| biosynthetic process | | CERS4, ALDH3A2 |
| IPR016439: Longevity assurance, | 3 | CERS2, CERS6, CERS4 |
| LAG1/LAC1 | | |
| PIR5F005225: longevity assurance | 3 | CERS2, CERS6, CERS4 |
| protein LAG1/LAC1 | | |
| GO: 0046513~ceramide biosynthetic | 6 | SAMD8, SPTLC2, CERS2, CERS6, SPTSSA, CERS4 |
| process | | |
| GO: 0050291~sphingosine N- | 3 | CERS2, CERS6, CERS4 |
| acyltransferase activity | | |
| hsa00600: Sphingolipid metabolism | 8 | SPTLC2, GLA, CERS2, CERS6, KDSR, CERS4, CERK, ASAH1 |
| domain: TLC | 3 | CERS2, CERS6, CERS4 |
| SM00724: TLC | 3 | CERS2, CERS6, CERS4 |
| IPR006634: TRAM/LAG1/CLN8 | 3 | CERS2, CERS6, CERS4 |
| homology domain | | |
| Homeobox | 8 | HIPK1, CERS2, ZHX1, HIPK2, HOPX, CERS6, CERS4, ZEB1 |
| DNA-binding region: Homeobox | 3 | CERS2, CERS6, CERS4 |
| IPR001356: Homeodomain | 6 | CERS2, ZHX1, HOPX, CERS6, CERS4, ZEB1 |
| SM00389: HOX | 3 | ZHX1, HOPX, ZEB1 |
| Enrichment Score: | | |
| 0.41325609345633085 | | |
| IPR023214: HAD- | 15 | NT5C3A, CMAS, ATP11A, CECR5, LPIN1, PMM2, PGP, ATP13A1, ATP2B4, |
| like domain | | ATP2C1, ATP8B2, CTDSP1, ENOPH1, UBLCP1, NT5C |
| active site: 4- | 6 | ATP13A 1 , ATP2B4, ATP2C1, ATP11A, ATP8B2, CTDSP1 |
| aspartylphosphate intermediate | | |
| IPR018303: P-type ATPase, | 5 | ATP13A 1 , ATP2B4, ATP2C1, ATP11A, ATP8B2 |
| phosphorylation site | | |
| IPR023299: P-type ATPase, | 5 | ATP13A 1 , ATP2B4, ATP2C1, ATP11A, ATP8B2 |
| cytoplasmic domain N | | |
| IPR008250: P-type | 5 | ATP13A 1 , ATP2B4, ATP2C1, ATP11A, ATP8B2 |
| ATPase, A domain | | |
| IPR001757: Cation- | 5 | ATP13A 1 , ATP2B4, ATP2C1, ATP11A, ATP8B2 |
| transporting P-type ATPase | | |
| Enrichment Score: | | |
| 0.40955924999908333 | | |
| IPR016181: Acyl-CoA N- | 8 | NAT6, SAT2, MGEA5, NAT10, KAT6B, KAT5, NAT9, ATE1 |
| acyltransferase | | |
| GO: 0008080~N-acetyltransferase | 5 | ESCO1, NAT6, SAT2, NAT10, NAT9 |
| activity | | |
| domain: N-acetyltransferase | 4 | NAT6, SAT2, NAT10, NAT9 |
| IPR000182: GNAT domain | 4 | NAT6, SAT2, NAT10, NAT9 |
| Enrichment Score: | | |
| 0.4082289580528521 | | |
| IPR001180: Citron-like | 4 | TNIK, MAP4K1, VP539, WDR45 |
| SM00036: CNH | 3 | TNIK, MAP4K1, VPS39 |
| domain: CNH | 3 | TNIK, MAP4K1, VP539 |
| Enrichment Score: | | |
| 0.3922449877254785 | | |
| repeat: ANK 25 | 3 | ANKRD52, ANKRD17, ANKRD44 |
| repeat: ANK 24 | 3 | ANKRD52, ANKRD17, ANKRD44 |
| repeat: ANK 22 | 3 | ANKRD52, ANKRD17, ANKRD44 |
| repeat: ANK 23 | 3 | ANKRD52, ANKRD17, ANKRD44 |
| repeat: ANK 20 | 3 | ANKRD52, ANKRD17, ANKRD44 |
| repeat: ANK 21 | 3 | ANKRD52, ANKRD17, ANKRD44 |
| repeat: ANK 7 | 9 | ANKRD52, ANKRD17, ANKRD44, NFKBIZ, EHMT1, MIB2, BCL3, FEM1B, |
| | | FEM1A |
| repeat: ANK 17 | 3 | ANKRD52, ANKRD17, ANKRD44 |
| repeat: ANK 18 | 3 | ANKRD52, ANKRD17, ANKRD44 |
| repeat: ANK 19 | 3 | ANKRD52, ANKRD17, ANKRD44 |
| repeat: ANK 8 | 7 | ANKRD52, ANKRD17, ANKRD44, EHMT1, MIB2, FEM1B, FEM1A |
| repeat: ANK 16 | 3 | ANKRD52, ANKRD17, ANKRD44 |
| repeat: ANK 13 | 3 | ANKRD52, ANKRD17, ANKRD44 |
| repeat: ANK 14 | 3 | ANKRD52, ANKRD17, ANKRD44 |
| repeat: ANK 15 | 3 | ANKRD52, ANKRD17, ANKRD44 |
| repeat: ANK 12 | 3 | ANKRD52, ANKRD17, ANKRD44 |
| repeat: ANK 9 | 5 | ANKRD52, ANKRD17, ANKRD44, MIB2, FEM1A |
| repeat: ANK 11 | 3 | ANKRD52, ANKRD17, ANKRD44 |
| repeat: ANK 10 | 3 | ANKRD52, ANKRD17, ANKRD44 |
| Enrichment Score: | | |
| 0.38403808705196346 | | |
| domain: CRIB | 4 | CDC42SE1, WASL, CDC42EP3, WAS |
| IPR000095: PAK-box/P21-Rho- | 4 | CDC42SE1, WASL, CDC42EP3, WAS |
| binding | | |
| SM00285: PBD | 3 | WASL, CDC42EP3, WAS |
| Enrichment Score: | | |
| 0.37683206426825694 | | |
| Host cell receptor | 9 | ICAM1, LAMP1, CD55, CXCR4, SLC20A2, IDE, HSPA1A, SLC52A2, ITGB1 |
| for virus entry | | |
| GO: 0001618~virus | 10 | ICAM1, LAMP1, CD55, CXCR4, SLC20A2, IDE, HSPA1A, SLC52A2, ITGB1, |
| receptor activity | | DPP4 |
| GO: 0046718~vira1 | 11 | ICAM1, LAMP1, CD55, SLC20A2, IDE, CD81, DYNLT1, HSPA1A, SLC52A2, |
| entry into host cell | | ITGB1, DPP4 |
| Enrichment Score: | | |
| 0.37484614935515076 | | |
| SM00461: WH1 | 3 | EVL, WASL, WAS |
| IPR000697: EVH1 | 3 | EVL, WASL, WAS |
| domain: WH1 | 3 | EVL, WASL, WAS |
| GO: 0008154~actin polymerization or | 3 | EVL, WASL, WAS |
| depolymerization | | |
| GO: 0007015~actin filament | 9 | NCK2, BCL2, PRKCI, BIN3, EVL, WASL, RHOF, WAS, WHAMM |
| organization | | |
| Enrichment Score: | | |
| 0.3728731212996016 | | |
| h_eif4Pathway: Regulation of | 6 | MAPK1, EIF4G3, EIF4E, PTEN, PIK3R1, PRKCB |
| eIF4e and p70 S6 Kinase | | |
| h_igf1mtorPathway: Skeletal | 5 | EIF4E, GSK3B, PEEN, PIK3R1, EIF2B5 |
| muscle hypertrophy is regulated via | | |
| AKT/mTOR pathway | | |
| h_mtorPathway: mTOR Signaling | 5 | EIF4G3, EIF4E, TSC1, PTEN, PIK3R1 |
| Pathway | | |
| Enrichment Score: | | |
| 0.3673728728107017 | | |
| GO: 1902187~negative regulation of | 5 | CHMP3, PML, TRIM27, TRIM26, TRIM25 |
| viral release from host cell | | |
| GO: 0070206~protein trimerization | 3 | TRIM4, TRIM27, TRIM22 |
| SM00449: SPRY | 13 | TRIM4, TRIM38, BTN3A1, ASH2L, RSPRY1, TRIM69, SPSB3, TRIM27, |
| | | TRIM14, TRIM26, TRIM25, TRIM22, SPRYD4 |
| domain: B30.2/SPRY | 13 | TRIM4, TRIM38, BTN3A1, ASH2L, RSPRY1, TRIM69, SPSB3, TRIM27, |
| | | TRIM14, TRIM26, TRIM25, TRIM22, SPRYD4 |
| IPR003877: SPla/RY | 13 | TRIM4, TRIM38, BTN3A1, ASH2L, RSPRY1, TRIM69, SPSB3, TRIM27, |
| anodine receptor SPRY | | TRIM14, TRIM26, TRIM25, TRIM22, SPRYD4 |
| IPR003879: Butyrophylin-like | 10 | TRIM4, TRIM38, BTN3A1, TRIM69, TRIM14, TRIM27, TRIM26, TRIM25, |
| | | TRIM22, SPRYD4 |
| SM00589: PRY | 7 | TRIM38, BTN3A1, TRIM69, TRIM14, TRIM27, TRIM26, TRIM25 |
| IPR001870: B30.2/SPRY domain | 13 | TRIM4, TRIM38, BTN3A1, ASH2L, RSPRY1, TRIM69, SPSB3, TRIM27, |
| | | TRIM14, TRIM26, TRIM25, TRIM22, SPRYD4 |
| IPR006574: SPRY-associated | 7 | TRIM38, BTN3A1, TRIM69, TRIM14, TRIM27, TRIM26, TRIM25 |
| zinc finger region: B box-type | 8 | TRIM4, TRIM38, TRIM14, TRIM27, RBCK1, TRIM26, TRIM22, MYCBP2 |
| SM00336: BBOX | 8 | TRIM4, TRIM38, TRIM33, TRIM14, PML, TRIM27, TRIM26, TRIM22 |
| IPRO00315: Zinc | 9 | TRIM4, TRIM38, TRIM33, TRIM69, TRIM14, PML, TRIM27, TRIM26, |
| finger, B-box | | TRIM22 |
| IPR013320: Concanavalin A-like | 18 | TSPEAR, SPSB3, NELL2, CLSTN1, TRIM27, LRBA, TRIM14, TRIM26, |
| lectin/glucanase, subgroup | | TRIM25, TRIM22, SPRYD4, LGALS9, TRIM4, TRIM38, BTN3A1, ASH2L, |
| | | TRIM69, RSPRY1 |
| Enrichment Score: | | |
| 0.3661268358100964 | | |
| hsa04611: Platelet | 18 | GNA13, ORAI1, ARHGEF1, ROCK1, ADCY7, ROCK2, PRKCI, STIM1, |
| activation | | APBB1IP, PPP1CB, ITGB1, ITPR2, MAPK1, MAPK13, SNAP23, PRKACB, |
| | | PIK3R1, AKT2 |
| hsa05205: Proteoglycans in | 26 | ITGB1, KRAS, TIAM1, SOS1, RAC1, NUDT16L1, PRKACB, MSN, PIK3R1, |
| cancer | | AKT2, ARHGEF1, ROCK1, ROCK2, CBL, RAF1, CD63, PPP1CB, FLNA, |
| | | PRKCB, ITPR2, CTSL, NRAS, MAPK1, CBLB, MAPK13, ARAF |
| hsa04270: Vascular | 14 | GNA13, ARHGEF1, ROCK1, ADCY7, ROCK2, PRKCH, RAF1, PPP1CB, |
| smooth muscle contraction | | PRKCD, PRKCB, ITPR2, MAPK1, ARAF, PRKACB |
| Enrichment Score: | | |
| 0.3655181734902265 | | |
| IPR004088: K | 7 | ANKRD17, FMR1, KHSRP, MEX3C, EXOSC3, QKI, FXR2 |
| Homology domain, type 1 | | |
| SM00322: KH | 6 | ANKRD17, FMR1, KHSRP, MEX3C, QKI, FXR2 |
| IPR004087: K Homology domain | 6 | ANKRD17, FMR1, KHSRP, MEX3C, QKI, FXR2 |
| domain: KH 2 | 4 | FMR1, KHSRP, MEX3C, FXR2 |
| domain: KH 1 | 4 | FMR1, KHSRP, MEX3C, FXR2 |
| Enrichment Score: | | |
| 0.3643845176662146 | | |
| Electron transport | 17 | ENOX2, UQCRC1, NDUFB7, NDUFA9, TXN2, CYB5A, NDUFA10, |
| | | UQCRFS1, GLRX2, NDUFV3, SDHA, SDHC, NDUFV2, TXNRD1, NDUFS1, |
| | | ETFA, GLRX |
| hsa05016: Huntington′s | 27 | UQCRC1, NDUFB7, TBP, CLTC, UQCRFS1, POLR2B, TBPL2, SIN3A, |
| disease | | CASP8, ATP5H, NDUFS1, TBPL1, NDUFA9, CREB1, CREBBP, NDUFA10, |
| | | PPARGC1A, SDHA, PPIF, NDUFV3, NRF1, EP300, SP1, BBC3, AP2A1, |
| | | SDHC, NDUFV2 |
| hsa05010: Alzheimer′s | 24 | UQCRC1, APH1A, NDUFB7, NDUFA9, IDE, FADD, BAD, NDUFA10, |
| disease | | UQCRFS1, NAE1, ITPR2, NDUFV3, ATF6, SDHA, TNFRSF1A, MAPK1, |
| | | CASP7, GSK3B, SDHC, CASP8, NDUFV2, PPP3CB, ATP5H, NDUFS1 |
| GO: 0032981~mitochondrial respiratory | 10 | NDUFAF4, NDUFV3, TIMMDC1, NDUFB7, AIFM1, NDUFA9, NDUFV2, |
| chain complex I assembly | | ECSIT, NDUFA10, NDUFS1 |
| Respiratory chain | 8 | NDUFV3, UQCRC1, NDUFB7, NDUFA9, NDUFV2, UQCRFS1, NDUFA10, |
| | | NDUFS1 |
| hsa00190: Oxidative | 16 | COX11, UQCRC1, NDUFB7, NDUFA9, ATP6V1H, NDUFA10, UQCRFS1, |
| phosphorylation | | ATP6V1F, NDUFV3, SDHA, SDHC, ATP6V1E1, NDUFV2, ATP6V0D1, |
| | | ATP5H, NDUFS1 |
| GO: 0005747~mitochondrial | 6 | NDUFV3, NDUFB7, NDUFA9, NDUFV2, NDUFA10, NDUFS1 |
| respiratory chain complex I | | |
| GO: 0008137~NADH dehydrogenase | 6 | NDUFV3, NDUFB7, NDUFA9, NDUFV2, NDUFA10, NDUFS1 |
| (ubiquinone) activity | | |
| hsa05012: Parkinson′s | 16 | UQCRC1, NDUFB7, NDUFA9, UBE2G1, UBE2J1, UBE2J2, NDUFA10, |
| disease | | UQCRFS1, NDUFV3, PPIF, SDHA, SDHC, NDUFV2, PRKACB, ATP5H, |
| | | NDUFS1 |
| GO: 0006120~mitochondrial electron | 6 | NDUFV3, NDUFB7, NDUFA9, NDUFV2, NDUFA10, NDUFS1 |
| transport, NADH to ubiquinone | | |
| Ubiquinone | 3 | NDUFV2, NDUFA10, NDUFS1 |
| Enrichment Score: | | |
| 0.35390729447514874 | | |
| IPR000209: Peptidase S8/S53 domain | 4 | TPP1, TPP2, PCSK7, FURIN |
| IPR023828: Peptidase S8, subtilisin, | 3 | TPP2, PCSK7, FURIN |
| Ser-active site | | |
| IPR022398: Peptidase S8, subtilisin, | 3 | TPP2, PCSK7, FURIN |
| His-active site | | |
| IPR015500: Peptidase S8, subtilisin- | 3 | TPP2, PCSK7, FURIN |
| related | | |
| IPR009020: Proteinase inhibitor, | 3 | TPP1, PCSK7, FURIN |
| propeptide | | |
| Serine protease | 9 | LONP1, PARL, TPP1, TPP2, GZMB, PCSK7, RHBDD1, FURIN, DPP4 |
| active site: Charge | 11 | APEH, CES2, ABHD17B, TPP1, TPP2, ABHD3, ABHD2, GZMB, PCSK7, |
| relay system | | FURIN, DPP4 |
| GO: 0004252~serine-type | 14 | GZMB, RHBDD1, FURIN, RHBDD2, IMMP1L, CTSL, APEH, LONP1, PARL, |
| endopeptidase activity | | TPP1, TPP2, CTSC, PCSK7, DPP4 |
| Enrichment Score: | | |
| 0.3488604922902843 | | |
| SM00849: SM00849 | 3 | HAGH, ETHE1, CPSF3 |
| IPR001279: Beta-lactamase-like | 4 | HAGH, ELAC2, ETHE1, CPSF3 |
| metal ion-binding site: Zinc 1 | 8 | HAGH, EHMT1, ETHE1, ARAF, PML, RAF1, USP16, CPSF3 |
| Enrichment Score: | | |
| 0.3388633674945358 | | |
| GO: 0005851~eukaryotic translation | 3 | EIF2B1, EIF2B4, EIF2B5 |
| initiation factor 2B complex | | |
| GO: 0043434~response | 9 | CD55, BSG, CDKN1B, BTG2, SOCS1, ANXA1, EIF2B1, EIF2B4, EIF2B5 |
| to peptide hormone | | |
| h_vegfPathway: VEGF, Hypoxia, | 7 | VHL, ELAVL1, EIF2B1, PIK3R1, EIF2B4, PRKCB, EIF2B5 |
| and Angiogenesis | | |
| GO: 0014003~oligodendrocyte | 3 | EIF2B1, EIF2B4, EIF2B5 |
| development | | |
| Leukodystrophy | 3 | EIF2B1, EIF2B4, EIF2B5 |
| GO: 0009408~response to heat | 6 | SOC53, HSPA1A, MAP2K7, EIF2B1, EIF2B4, EIF2B5 |
| Enrichment Score: | | |
| 0.3332483915134039 | | |
| IPR020103: Pseudo-uridine synthase, | 3 | RPUSD3, TRUB2, PUS7 |
| catalytic domain | | |
| GO: 0009982~pseudo-uridine synthase | 3 | RPUSD3, TRUB2, PUS7 |
| activity | | |
| GO: 0001522~pseudo-uridine synthesis | 3 | RPUSD3, TRUB2, PUS7 |
| Enrichment Score: | | |
| 0.3283640348823858 | | |
| SM00312: PX | 7 | SNX19, SNX29, PIK3C2A, SNX2, SNX4, SNX13, SNX11 |
| GO: 0035091~phosphatidylinositol | 12 | SNX19, SH3YL1, SNX29, ING2, PIK3C2A, PASK, SNX2, SNX4, PITPNC1, |
| binding | | SNX13, SNX11, ITPR2 |
| domain: PX | 7 | SNX19, SNX29, PIK3C2A, SNX2, SNX4, SNX13, SNX11 |
| IPR001683: Phox | 7 | SNX19, SNX29, PIK3C2A, SNX2, SNX4, SNX13, SNX11 |
| homologous domain | | |
| GO: 0016050~vesicle organization | 4 | SNX2, SNX4, WASL, SNX11 |
| Enrichment Score: | | |
| 0.3248740734594386 | | |
| 72JAP_inter- | 5 | TNFRSF1A, XIAP, CASP7, CASP8, FADD |
| action_with_cell_death_pathways | | |
| h_mitochondriaPathway: Role of | 5 | XIAP, AIFM1, CASP7, BCL2, CASP8 |
| Mitochondria in Apoptotic Signaling | | |
| h_caspasePathway: | 5 | XIAP, LMNB2, CASP7, CASP8, GZMB |
| Caspase Cascade in Apoptosis | | |
| Enrichment Score: | | |
| 0.3159225008635777 | | |
| GO: 0004859~phospholipase | 3 | ANXA1, ANXA5, ANXA2 |
| inhibitor activity | | |
| Annexin | 3 | ANXA1, ANXA5, ANXA2 |
| SM00335: ANX | 3 | ANXA1, ANXA5, ANXA2 |
| IPR018502: Annexin repeat | 3 | ANXA1, ANXA5, ANXA2 |
| IPR018252: Annexin repeat, | 3 | ANXA1, ANXA5, ANXA2 |
| conserved site | | |
| IPR001464: Annexin | 3 | ANXA1, ANXA5, ANXA2 |
| Calcium/phospholipid-binding | 3 | ANXA1, ANXA5, ANXA2 |
| repeat: Annexin 1 | 3 | ANXA1, ANXA5, ANXA2 |
| repeat: Annexin 3 | 3 | ANXA1, ANXA5, ANXA2 |
| repeat: Annexin 2 | 3 | ANXA1, ANXA5, ANXA2 |
| repeat: Annexin 4 | 3 | ANXA1, ANXA5, ANXA2 |
| GO: 0005544~calcium-dependent | 7 | C2CD5, SYT11, ANXA1, CPNE1, SYTL3, ANXA5, ANXA2 |
| phospholipid binding | | |
| Enrichment Score: | | |
| 0.31228187414723185 | | |
| h_cxcr4Pathway: CX CR4 Signaling | 6 | MAPK1, CXCR4, RELA, RAF1, PIK3R1, PRKCB |
| Pathway | | |
| h_eif4Pathway: Regulation of eIF4e | 6 | MAPK1, EIF4G3, EIF4E, PTEN, PIK3R1, PRKCB |
| and p70 S6 Kinase | | |
| h_edg1Pathway: Phospholipids as | 5 | MAPK1, RAC1, PIK3R1, ASAH1, PRKCB |
| signalling intermediaries | | |
| hsa04960: Aldosterone-regulated | 4 | MAPK1, KRAS, PIK3R1, PRKCB |
| sodium reabsorption | | |
| Enrichment Score: | | |
| 0.3071399535752837 | | |
| SM00323: RasGAP | 3 | IQGAP2, RASA1, RASA2 |
| IPR023152: Ras GTPase-activating | 3 | IQGAP2, RASA1, RASA2 |
| protein, conserved site | | |
| domain: Ras-GAP | 3 | IQGAP2, RASA1, RASA2 |
| IPR001936: Ras | 3 | IQGAP2, RASA1, RASA2 |
| GTPase-activating protein | | |
| Enrichment Score: | | |
| 0.2948433234633509 | | |
| IPR000225: Armadillo | 7 | USO1, KPNA6, ARMCX3, ARMC6, KPNA1, ARMC1, APC |
| repeat: ARM 3 | 6 | USO1, KPNA6, ARMCX3, ARMC6, KPNA1, APC |
| repeat: ARM 2 | 6 | USO1, KPNA6, ARMCX3, ARMC6, KPNA1, APC |
| repeat: ARM 4 | 5 | USO1, KPNA6, ARMC6, KPNA1, APC |
| repeat: ARM 7 | 4 | USO1, KPNA6, KPNA1, APC |
| repeat: ARM 6 | 4 | USO1, KPNA6, KPNA1, APC |
| repeat: ARM 9 | 3 | USO1, KPNA6, KPNA1 |
| repeat: ARM 5 | 4 | USO1, KPNA6, KPNA1, APC |
| SM00185: ARM | 5 | USO1, KPNA6, ARMC6, KPNA1, APC |
| repeat: ARM 1 | 4 | USO1, ARMCX3, ARMC6, APC |
| repeat: ARM 8 | 3 | USO1, KPNA6, KPNA1 |
| Enrichment Score: | | |
| 0.29256414347139453 | | |
| IPR010920: Like-Sm (LSM) domain | 5 | LSM14A, LSM14B, LSM3, LSM10, LSM1 |
| SM00651: Sm | 3 | LSM3, LSM10, LSM1 |
| IPR001163: Ribonucleoprotein | 3 | LSM3, LSM10, LSM1 |
| LSM domain | | |
| Enrichment Score: | | |
| 0.28416826259197325 | | |
| SM00450: RHOD | 4 | DUSP4, DUSP16, TSTD1, MPST |
| IPR001763: Rhodanese-like domain | 4 | DUSP4, DUSP16, TSTD1, MPST |
| domain: Rhodanese | 3 | DUSP4, DUSP16, TSTD1 |
| Enrichment Score: | | |
| 0.2825610876287823 | | |
| hsa00061: Fatty acid biosynthesis | 4 | FASN, ACSL4, ACSL3, ACSL5 |
| GO: 0102391~decanoate--CoA | 3 | ACSL4, ACSL3, ACSL5 |
| ligase activity | | |
| hsa00071: Fatty acid | 8 | ECI1, ECI2, ACSL4, ACAT2, ACSL3, ALDH3A2, ALDH9A1, ACSL5 |
| degradation | | |
| GO: 0004467~long-chain fatty acid- | 3 | ACSL4, ACSL3, ACSL5 |
| CoA ligase activity | | |
| GO: 0035338~long-chain fatty-acyl- | 6 | ELOVL1, ACOT9, FASN, ACSL4, ACSL3, ACSL5 |
| CoA biosynthetic process | | |
| GO: 0001676~long- | 3 | ACSL4, ACSL3, ACSL5 |
| chain fatty acid metabolic process | | |
| hsa03320: PPAR signaling pathway | 7 | RXRB, ILK, ACSL4, PCK2, ACSL3, SCP2, ACSL5 |
| IPR020845: AMP-binding, conserved | 3 | ACSL4, ACSL3, ACSL5 |
| site | | |
| hsa01212: Fatty acid metabolism | 5 | FASN, ACSL4, ACAT2, ACSL3, ACSL5 |
| Fatty acid | 11 | ECI1, ELOVL1, PRKAG2, PRKAB1, FASN, PRKAA1, ACSL4, LPIN1, ACSL3, |
| metabolism | | ACSL5, HSD17B8 |
| IPR000873: AMP-dependent | 3 | ACSL4, ACSL3, ACSL5 |
| synthetase/ligase | | |
| Enrichment Score: | | |
| 0.2825385029515912 | | |
| GO: 0051056~regulation of small | 19 | ARHGEF3, VAV3, RALBP1, RALGAPB, ARHGAP17, MYO9B, ARHGAP15, |
| GTPase mediated | | VAV1, FAM13B, ARHGAP30, RALGAPA1, TIAM1, SOS1, SIPA1L1, RAC1, |
| signal transduction | | RHOT1, RHOT2, ARAP2, RHOF |
| SM00324: RhoGAP | 8 | ARHGAP30, RALBP1, MYO9B, ARHGAP17, ARHGAP15, ARAP2, PIK3R1, |
| | | FAM13B |
| IPR008936: Rho | 11 | ARHGAP30, RALBP1, IQGAP2, MYO9B, ARHGAP17, ARHGAP15, ARAP2, |
| GTPase activation protein | | PIK3R1, RASA1, FAM13B, RASA2 |
| domain: Rho-GAP | 8 | ARHGAP30, RALBP1, MYO9B, ARHGAP17, ARHGAP15, ARAP2, PIK3R1, |
| | | FAM13B |
| IPR000198: Rho | 8 | ARHGAP30, RALBP1, MYO9B, ARHGAP17, ARHGAP15, ARAP2, PIK3R1, |
| GTPase-activating protein domain | | FAM13B |
| Enrichment Score: | | |
| 0.27812245787260176 | | |
| repeat: ANK 7 | 9 | ANKRD52, ANKRD17, ANKRD44, NFKBIZ, EHMT1, MD32, BCL3, FEM1B, |
| | | FEM1A |
| repeat: ANK 3 | 25 | CAMTA2, OSTF1, NFKBID, NFKB113, FEM1B, FEM1A, RFXANK, |
| | | ANKRD36, ANKRD52, ANKRD17, GABPB1, ILK, ANKRD37, BCL3, |
| | | HECTD1, IBTK, NFKBIZ, ANKS1A, EHMT1, ANKRD44, ACAP1, KRIT1, |
| | | MIB2, ACAP2, BARD1 |
| repeat: ANK 1 | 29 | CAMTA2, OSTF1, NFKBID, NFKBIB, ASAP1, FEM1B, FEM1A, RFXANK, |
| | | ANKRD36, ANKRD52, ANKRD17, GABPB1, ILK, ANKRD37, BCL3, |
| | | HECTD1, IBTK, NFKBIZ, ANKS1A, EHMT1, GPANK1, ANKRD40, |
| | | ANKRD44, ACAP1, MIB2, KRIT1, ACAP2, DGKZ, BARD1 |
| repeat: ANK 2 | 29 | CAMTA2, OSTF1, NFKBID, NFKBIB, ASAP1, FEM1B, FEM1A, RFXANK, |
| | | ANKRD36, ANKRD52, ANKRD17, GABPB1, ILK, ANKRD37, BCL3, |
| | | HECTD1, IBTK, NFKBIZ, ANKS1A, EHMT1, GPANK1, ANKRD40, |
| | | ANKRD44, ACAP1, MIB2, KRIT1, ACAP2, DGKZ, BARD1 |
| repeat: ANK 6 | 12 | ANKRD52, ANKRD17, ANKRD44, NFKBIZ, ANKS1A, EHMT1, NFKBID, |
| | | MIB2, NFKBIB, BCL3, FEM1B, FEM1A |
| ANK repeat | 29 | CAMTA2, OSTF1, NFKBID, NFKBIB, ASAP1, FEM1B, FEM1A, RFXANK, |
| | | ANKRD36, ANKRD52, ANKRD17, GABPB1, ILK, ANKRD37, BCL3, |
| | | HECTD1, IBTK, NFKBIZ, ANKS1A, EHMT1, GPANK1, ANKRD40, |
| | | ANKRD44, ACAP1, MIB2, KRIT1, ACAP2, DGKZ, BARD1 |
| SM00248: ANK | 27 | OSTF1, NFKBID, NFKBIB, ASAP1, FEM1B, FEM1A, RFXANK, ANKRD36, |
| | | ANKRD52, ANKRD17, GABPB1, ILK, ANKRD37, BCL3, HECTD1, IBTK, |
| | | NFKBIZ, ANKS1A, EHMT1, ANKRD40, ANKRD44, ACAP1, MIB2, KRIT1, |
| | | ACAP2, DGKZ, BARD1 |
| IPR020683: Ankyrin | 29 | CAMTA2, OSTF1, NFKBID, NFKBIB, ASAP1, FEM1B, FEM1A, RFXANK, |
| repeat-containing | | ANKRD36, ANKRD52, ANKRD17, GABPB1, ILK, ANKRD37, BCL3, |
| domain | | HECTD1, IBTK, NFKBIZ, ANKS1A, EHMT1, GPANK1, ANKRD40, |
| | | ANKRD44, ACAP1, MIB2, KRIT1, ACAP2, DGKZ, BARD1 |
| repeat: ANK 4 | 18 | NFKBIZ, ANKS1A, EHMT1, NFKBID, NFKBIB, FEM1B, FEM1A, RFXANK, |
| | | ANKRD36, ANKRD52, ANKRD17, GABPB1, ANKRD44, MIB2, KRIT1, ILK, |
| | | BCL3, HECTD1 |
| repeat: ANK 5 | 15 | NFKBIZ, ANKS1A, EHMT1, NFKBID, NFKBIB, FEM1B, RFXANK, FEM1A, |
| | | ANKRD52, ANKRD17, ANKRD44, GABPB1, MIB2, ILK, BCL3 |
| IPR002110: Ankyrin | 27 | OSTF1, NFKBID, NFKBIB, ASAP1, FEM1B, FEM1A, RFXANK, ANKRD36, |
| repeat | | ANKRD52, ANKRD17, GABPB1, ILK, ANKRD37, BCL3, HECTD1, IBTK, |
| | | NFKBIZ, ANKS1A, EHMT1, ANKRD40, ANKRD44, ACAP1, MIB2, KRIT1, |
| | | ACAP2, DGKZ, BARD1 |
| Enrichment Score: | | |
| 0.2668097961653456 | | |
| GO: 0042276~error-prone translesion | 4 | RPA1, POLK, RFC2, REV3L |
| synthesis | | |
| DNA replication | 12 | RPA1, POLK, RBBP4, RFC2, WRNIP1, FAM111A, RRM1, CINP, ORC4, |
| | | POLB, MCM6, REV3L |
| GO: 0006297~nucleotide-excision | 4 | RPA1, POLK, RFC2, POLB |
| repair, DNA gap filling | | |
| GO: 0019985~translesion synthesis | 5 | RPA1, POLK, RFC2, TRIM25, REV3L |
| hsa03460: Fanconi anemia pathway | 5 | WDR48, RPA1, RAD51C, POLK, REV3L |
| Enrichment Score: | | |
| 0.25720288166374317 | | |
| GO: 0004114~3′,5′-cyclic-nucleotide | 5 | PDE6D, PDE7A, PDE4B, PDE4D, RUNX1 |
| phosphodiesterase activity | | |
| IPR002073: 3′,5′-cyclic nucleotide | 4 | PDE7A, PDE4B, PDE4D, RUNX1 |
| phosphodiesterase, catalytic domain | | |
| cAMP | 5 | PRKAR2A, PDE7A, PDE4B, PDE4D, PRKACB |
| GO: 0004115-3′,5′-cyclic- | 3 | PDE7A, PDE4B, PDE4D |
| AMP phosphodiesterase activity | | |
| GO: 0006198~cAMP catabolic process | 3 | PDE7A, PDE4B, PDE4D |
| metal ion-binding | 4 | PTER, PDE7A, PDE4B, PDE4D |
| site: Divalent metal cation 1 | | |
| metal ion-binding | 4 | PTER, PDE7A, PDE4B, PDE4D |
| site: Divalent metal cation 2 | | |
| IPR023088: 3′,5′- | 3 | PDE7A, PDE4B, PDE4D |
| cyclic nucleotide phosphodiesterase | | |
| IPRO23174: 3′,5′-cyclic nucleotide | 3 | PDE7A, PDE4B, PDE4D |
| phosphodiesterase, conserved site | | |
| hsa05032: Morphine addiction | 6 | ADCY7, PDE7A, PDE4B, PDE4D, PRKACB, PRKCB |
| Enrichment Score: | | |
| 0.24930966068454638 | | |
| SM00326: SH3 | 24 | FYB, DBNL, OSTF1, VAV3, STAM2, MPP6, ASAP1, VAV1, NCK2, DOCK2, |
| | | SH3YL1, CRKL, SH3GLB2, LASP1, PSTPIP1, STAM, UBASH3A, GRAP2, |
| | | BIN1, ABL2, RASA1, SASH3, PIK3R1, MATK |
| SH3 domain | 24 | FYB, DBNL, OSTF1, VAV3, STAM2, MPP6, ASAP1, VAV1, NCK2, DOCK2, |
| | | SH3YL1, CRKL, SH3GLB2, LASP1, PSTPIP1, STAM, UBASH3A, GRAP2, |
| | | BIN1, ABL2, RASA1, SASH3, PIK3R1, MATK |
| domain.SH3 | 19 | FYB, DBNL, OSTF1, STAM2, MPP6, ASAP1, DOCK2, SH3YL1, SH3GLB2, |
| | | LASP1, PSTPIP1, UBASH3A, STAM, BIN1, ABL2, RASA1, SASH3, PIK3R1, |
| | | MATK |
| IPR001452: Src | 24 | FYB, DBNL, OSTF1, VAV3, STAM2, MPP6, ASAP1, VAV1, NCK2, DOCK2, |
| homology-3 domain | | SH3YL1, CRKL, SH3GLB2, LASP1, PSTPIP1, STAM, UBASH3A, GRAP2, |
| | | BIN1, ABL2, RASA1, SASH3, PIK3R1, MATK |
| Enrichment Score: | | |
| 0.24872976470051114 | | |
| GO: 0004004~ATP- | 9 | DDX23, DHX29, DDX19A, DHX34, DDX50, DHX16, DDX10, DDX51, |
| dependent RNA helicase activity | | DDX42 |
| IPR011709: Domain | 3 | DHX29, DHX34, DHX16 |
| of unknown function DUF1605 | | |
| SM00847: SM00847 | 3 | DHX29, DHX34, DHX16 |
| IPR007502: Helicase-associated domain | 3 | DHX29, DHX34, DHX16 |
| Enrichment Score: | | |
| 0.23256970544677066 | | |
| binding site: NADP | 5 | G6PD, AKR7A2, IDH2, GRHPR, DCXR |
| nucleotide phosphate-binding | 9 | HSD17B11, HTATIP2, G6PD, AKR1B1, AKR7A2, IDH2, KDSR, GRHPR, |
| region: NADP | | DCXR |
| NADP | 15 | HSD17B11, HTATIP2, GLUD2, PYROXD1, GRHPR, FAR1, G6PD, AKR1B1, |
| | | AKR7A2, FASN, IDH2, KDSR, TXNRD1, DCXR, CRYZL1 |
| Enrichment Score: | | |
| 0.23055832452848027 | | |
| GO: 0070125~mitochondrial | 14 | MRPL42, MRPS14, MRPS23, MRPS25, MRPS11, GFM2, MRPS9, TSFM, |
| translational elongation | | GFM1, MRPL16, MRPL55, MRPL34, MRPL44, MRPL35 |
| GO: 0070126~mitochondrial | 13 | MRPL42, MRPS23, MRPS14, MRPS25, MRPS11, MRRF, GFM2, MRPS9, |
| translational termination | | MRPL16, MRPL55, MRPL34, MRPL44, MRPL35 |
| GO: 0005763~mitochondrial small | 5 | MRPL42, MRPS9, MRPS14, MRPS11, MRPS2 |
| ribosomal subunit | | |
| Ribonucleoprotein | 31 | RALY, RPL17, MRPL42, MRPS14, MRPS11, LARP1B, HNRNPLL, LSM14A, |
| | | LSM14B, MRPL16, MRPL55, AGO2, LSM3, L SM1, MRPL34, MRPL35, |
| | | MRPS23, RXRB, MRPS25, EFTUD2, FMR1, MRPS2, RPS6KA3, HNRNPH2, |
| | | MRPS9, LSM10, PARP4, CPSF3, METTL17, MVP, MRPL44 |
| GO: 0006412~translation | 25 | RPL17, MRPL42, MRPS14, MRPS11, HBS1L, EIF4EBP2, MRPL16, AGO2, |
| | | MRPL55, SLC25A28, MRPL34, MRPL35, MRPS23, EFTUD2, GTF2H3, |
| | | MRRF, MRPS2, SLC25A32, MRPS9, SLC25A38, FARSB, YARS2, SLC25A16, |
| | | SLC25A53, METTL17 |
| Ribosomal protein | 16 | RPL17, MRPL42, MRPS14, MRPS23, RXRB, MRPS25, MRPS11, MRPS2, |
| | | RPS6KA3, MRPS9, MRPL16, MRPL55, MRPL34, METTL17, MRPL44, |
| | | MRPL35 |
| GO: 0005840~ribosome | 15 | MRPL42, MRPS14, MRPS23, RXRB, MRPS25, MRPS11, MRPS2, RPS6KA3, |
| | | MRPS9, MRPL16, MRPL55, MRPL34, METTL17, MRPL44, MRPL35 |
| GO: 0003735~structural constituent | 17 | RPL17, MRPL42, MRPS14, MRPS23, MRPS25, MRPS11, MRPS2, SLC25A32, |
| of ribosome | | MRPS9, MRPL16, SLC25A38, SLC25A28, MRPL55, SLC25A16, MRPL34, |
| | | SLC25A53, MRPL35 |
| hsa03010: Ribosome | 8 | RPL17, MRPS9, MRPS14, MRPL16, MRPS11, MRPL34, MRPS2, MRPL35 |
| Enrichment Score: | | |
| 0.23040649502848873 | | |
| repeat: WD 8 | 9 | WDR48, PHIP, TBL1XR1, EML3, ELP2, WDR6, TBL1X, PWP2, GEMIN5 |
| repeat: WD 13 | 3 | ELP2, PWP2, GEMIN5 |
| repeat: WD 11 | 4 | ELP2, WDR6, PWP2, GEMIN5 |
| repeat: WD 10 | 4 | ELP2, WDR6, PWP2, GEMIN5 |
| repeat: WD 12 | 3 | ELP2, PWP2, GEMIN5 |
| repeat: WD 9 | 5 | EML3, ELP2, WDR6, PWP2, GEMIN5 |
| Enrichment Score: | | |
| 0.2297546707824956 | | |
| GO: 0033572~transferrin transport | 7 | SLC11A2, ATP6V1E1, RAB11B, ATP6V1H, CLTC, ATP6V0D1, ATP6V1F |
| GO: 0016241~regulation of | 7 | CAPNS1, EXOC7, ATP6V1E1, ATP6V1H, MAPK8, ATP6V0D1, VPS26A |
| macroautophagy | | |
| hsa04721: Synaptic | 9 | DNM3, AP2A1, ATP6V1E1, ATP6V1H, NAPA, VAMP2, CLTC, ATP6V0D1, |
| vesicle cycle | | ATP6V1F |
| GO: 0046961~proton-transporting | 4 | ATP6V1E1, ATP6V1H, ATP6V0D1, ATP6V1F |
| ATPase activity, rotational mechanism | | |
| hsa05110: Vibriocholerae infection | 7 | ATP6V1E1, ATP6V1H, PRKACB, PDIA4, ATP6V0D1, ATP6V1F, PRKCB |
| GO: 0090383-phagosome acidification | 4 | ATP6V1E1, ATP6V1H, ATP6V0D1, ATP6V1F |
| GO: 0015991-ATP hydrolysis coupled | 4 | ATP6V1E1, ATP6V1H, ATP6V0D1, ATP6V1F |
| proton transport | | |
| GO: 0015078~hydrogen ion | 4 | SLC11A2, ATP6V0D1, ATP5H, ATP6V1F |
| transmembrane transporter activity | | |
| Hydrogen ion transport | 5 | ATP6V1E1, ATP6V1H, ATP6V0D1, ATP5H, ATP6V1F |
| hsa04966: Collecting duct acid secretion | 3 | ATP6V1E1, ATP6V0D1, ATP6V1F |
| GO: 0015992~proton transport | 4 | ATP6V1E1, HVCN1, ATP6V0D1, ATP6V1F |
| Enrichment Score: | | |
| 0.22901571749480087 | | |
| SM00156: PP2Ac | 3 | PPP2CB, PPP3CB, PPP1CB |
| IPR006186: Serine/threonine-specific | 3 | PPP2CB, PPP3CB, PPP1CB |
| protein phosphatase/bis(5- | | |
| nucleosyl)-tetraphosphatase | | |
| IPR004843: Metallophosphoesterase | 4 | PPP2CB, PPP3CB, DBR1, PPP1CB |
| domain | | |
| GO: 0004721~phosphoprotein | 6 | PGP, PPP2CB, DUSP16, PPP3CB, PTEN, PPP1CB |
| phosphatase activity | | |
| metal ion-binding site: Iron | 4 | PPP2CB, PPP3CB, EGLN1, PPP1CB |
| Enrichment Score: | | |
| 0.219815162312881 | | |
| domain.G-patch | 4 | CHERP, SUGP1, GPANK1, RBM10 |
| SM00443: G_patch | 4 | CHERP, SUGP1, GPANK1, RBM10 |
| IPR000467: G-patch domain | 4 | CHERP, SUGP1, GPANK1, RBM10 |
| Enrichment Score: | | |
| 0.21462490830549383 | | |
| SM00516: SEC14 | 4 | TTPAL, GDAP2, BNIP2, SEC14L1 |
| domain.CRAL-TRIO | 4 | TTPAL, GDAP2, BNIP2, SEC14L1 |
| IPR001251: CRAL-TRIO domain | 4 | TTPAL, GDAP2, BNIP2, SEC14L1 |
| Enrichment Score: | | |
| 0.21380660245611388 | | |
| GO: 0005385~zinc ion transmembrane | 4 | SLC11A2, SLC30A5, SLC39A6, SLC39A3 |
| transporter activity | | |
| GO: 0071577~zinc II | 3 | SLC30A5, SLC39A6, SLC39A3 |
| ion transmembrane transport | | |
| Zinc transport | 3 | SLC30A5, SLC39A6, SLC39A3 |
| Enrichment Score: | | |
| 0.2125851179351233 | | |
| domain: BTB | 18 | BACH2, ZBTB10, ZBTB11, ZNF131, ZBTB40, KCTD20, KEAP1, KCTD2, |
| | | IVNS1ABP, KCTD6, ZBTB38, SHKBP1, KBTBD2, KLHL9, KCTD18, ZBTB2, |
| | | KLHL24, SPOP |
| SM00225: BTB | 19 | IBTK, BACH2, ZBTB10, ZBTB11, ZNF131, BTBD7, ZBTB40, KCTD20, |
| | | KEAP1, KCTD2, IVNS1ABP, KCTD6, ZBTB38, SHKBP1, KBTBD2, KLHL9, |
| | | ZBTB2, KLHL24, SPOP |
| GO: 0031463~Cul3- | 8 | CUL3, KBTBD2, BACH2, KLHL9, KEAP1, KLHL24, KCTD2, SPOP |
| RING ubiquitin ligase complex | | |
| IPR000210: BTB/POZ-like | 19 | IBTK, BACH2, ZBTB10, ZBTB11, ZNF131, BTBD7, ZBTB40, KCTD20, |
| | | KEAP1, KCTD2, IVNS1ABP, KCTD6, ZBTB38, SHKBP1, KBTBD2, KLHL9, |
| | | ZBTB2, KLHL24, SPOP |
| IPR011333: BTB/POZ fold | 20 | IBTK, BACH2, ZBTB10, ZBTB11, ZNF131, BTBD7, ZBTB40, KCTD20, |
| | | KEAP1, KCTD2, IVNS1ABP, ZBTB38, KCTD6, SHKBP1, KBTBD2, KLHL9, |
| | | KCTD18, ZBTB2, KLHL24, SPOP |
| Enrichment Score: | | |
| 0.20376403779805952 | | |
| Aminopeptidase | 5 | LNPEP, TPP2, ERAP1, DPP4, DNPEP |
| GO: 0004177~amino peptidase activity | 4 | LNPEP, TPP2, ERAP1, DNPEP |
| GO: 0070006~metalloaminopeptidase | 3 | LNPEP, ERAP1, DNPEP |
| activity | | |
| GO: 0008237~metallopeptidase | 7 | STAMBP, LNPEP, CHMP1A, CNDP2, STAMBPL1, ERAP1, DNPEP |
| activity | | |
| Enrichment Score: | | |
| 0.195467663648754 | | |
| h_cdc42racPathway: Role of PI3K | 4 | ARPC1A, RAC1, WASL, PIK3R1 |
| subunit p85 in regulation of Actin | | |
| Organization and Cell Migration | | |
| h_salmonellaPathway: | 3 | ARPC1A, RAC1, WASL |
| How does salmonella hijack a cell | | |
| h_actinYPathway: Y branching of | 3 | ARPC1A, RAC1, WASL |
| actin filaments | | |
| Enrichment Score: | | |
| 0.19234790118499914 | | |
| GO: 0030676~Rac guanyl-nucleotide | 4 | DOCK2, VAV3, TIAM1, VAV1 |
| exchange factor activity | | |
| IPR001331: Guanine-nucleotide | 4 | ARHGEF3, VAV3, TIAM1, VAV1 |
| dissociation stimulator, CDC24, | | |
| conserved site | | |
| GO: 0035023~regulation of | 8 | ARHGEF3, VAV3, ARHGEF1, TIAM1, SOS1, RAF1, MYO9B, VAV1 |
| Rho protein signal transduction | | |
| domain: DH | 6 | ARHGEF3, VAV3, ARHGEF1, TIAM1, SOS1, VAV1 |
| SM00325: RhoGEF | 6 | ARHGEF3, VAV3, ARHGEF1, TIAM1, SOS1, VAV1 |
| IPR000219: Dbl homology (DH) | 6 | ARHGEF3, VAV3, ARHGEF1, TIAM1, SOS1, VAV1 |
| domain | | |
| GO: 0005089~Rho guanyl-nucleotide | 6 | ARHGEF3, VAV3, ARHGEF1, TIAM1, SOS1, VAV1 |
| exchange factor activity | | |
| Enrichment Score: | | |
| 0.1850282990952521 | | |
| region of interest: | 5 | NR1H2, RXRB, NR4A1, RORA, MR1 |
| Ligand-binding | | |
| IPR013088: Zinc | 8 | NR1H2, ESRRA, RXRB, GATA3, GATAD2A, NR4A1, RORA, RERE |
| finger, NHR/GATA-type | | |
| GO: 0030522~intracellular receptor | 6 | ESRRA, NCOA1, NCOA2, NR4A1, RORA, BRD8 |
| signaling pathway | | |
| GO: 0004879~RNA polymerase II | 5 | NR1H2, ESRRA, RXRB, NR4A1, RORA |
| transcription factor activity, ligand- | | |
| activated sequence-specific DNA | | |
| binding | | |
| GO: 0003707~steroid | 7 | NR1H2, ESRRA, RXRB, PGRMC2, NR4A1, ABHD2, RORA |
| hormone receptor activity | | |
| GO: 0043401~steroid | 7 | NR1H2, ESRRA, RXRB, PGRMC2, NR4A1, ABHD2, RORA |
| hormone mediated signaling pathway | | |
| DNA-binding | 5 | NR1H2, ESRRA, RXRB, NR4A1, RORA |
| region: Nuclear receptor | | |
| zinc finger region: NR C4-type | 5 | NR1H2, ESRRA, RXRB, NR4A1, RORA |
| SM00399: ZnF_C4 | 5 | NR1H2, ESRRA, RXRB, NR4A1, RORA |
| IPR001628: Zinc finger, nuclear | 5 | NR1H2, ESRRA, RXRB, NR4A1, RORA |
| hormone receptor-type | | |
| SM00430: HOLI | 5 | NR1H2, ESRRA, RXRB, NR4A1, RORA |
| IPR001723: Steroid | 5 | NR1H2, ESRRA, RXRB, NR4A1, RORA |
| hormone receptor | | |
| IPR000536: Nuclear hormone receptor, | 5 | NR1H2, ESRRA, RXRB, NR4A1, RORA |
| ligand-binding, core | | |
| Enrichment Score: | | |
| 0.18471285738684817 | | |
| IPR008984: SMAD/FHA domain | 7 | MDC1, SLMAP, FOXK2, APTX, TIFA, SMAD3, IRF3 |
| IPR000253: Forkhead- | 5 | MDC1, SLMAP, FOXK2, APTX, TIFA |
| associated (FHA) domain | | |
| SM00240: FHA | 3 | MDC1, SLMAP, FOXK2 |
| domain: FHA | 4 | MDC1, SLMAP, FOXK2, TIFA |
| Enrichment Score: | | |
| 0.18296343468740298 | | |
| GO: 2000503~positive regulation of | 3 | CCL3, XCL1, CCL4 |
| natural killer cell chemotaxis | | |
| GO: 0030593~neutrophil | 11 | CCL3, VAV3, GBF1, PDE4B, CKLF, IFNG, PDE4D, XCL1, VAV1, CCL4, |
| chemotaxis | | XCL2 |
| Chemotaxis | 11 | CCL3, IL16, CKLF, CMTM7, PLGRKT, CXCR3, XCL1, CMTM3, CCL4, |
| | | XCL2, LGALS9 |
| GO: 0002548~monocyte chemotaxis | 5 | CCL3, ANXA1, XCL1, CCL4, XCL2 |
| GO: 0050729~positive regulation of | 8 | TNFRSF1A, CCL3, MAPK13, JAK2, XCL1, CCL4, XCL2, IL2 |
| inflammatory response | | |
| GO: 0071346~cellular response to | 6 | CCL3, HLA-DPA1, XCL1, CCL4, XCL2, LGALS9 |
| interferon-gamma | | |
| GO: 0008009~chemokine activity | 5 | CCL3, CKLF, XCL1, CCL4, XCL2 |
| GO: 0070098~chemokine-mediated | 7 | CCL3, CXCR4, CCR4, CXCR3, XCL1, CCL4, XCL2 |
| signaling pathway | | |
| GO: 0071347~cellular response to | 8 | ICAM1, CCL3, RELA, PYCARD, RORA, XCL1, CCL4, XCL2 |
| interleukin-1 | | |
| SM00199: SCY | 4 | CCL3, XCL1, CCL4, XCL2 |
| IPR001811: Chemokine | 4 | CCL3, XCL1, CCL4, XCL2 |
| interleukin-8-like domain | | |
| GO: 0070374~positive regulation of | 13 | ICAM1, FBXW7, CCL3, JUN, PYCARD, RIPK2, DSTYK, PTPN22, XCL1, |
| ERK1 and ERK2 cascade | | PTEN, CCL4, XCL2, LGALS9 |
| Enrichment Score: | | |
| 0.17552852657218571 | | |
| Pharmaceutical | 9 | LIF, CSF2, GLA, SOCS3, IFNG, MS4A1, ANXA1, CTLA4, IL2 |
| h_stemPathway: Regulation of | 4 | CSF2, CD8A, CSF1, IL2 |
| hematopoiesis by cytokines | | |
| 88.Alternatively_Activated_APC | 3 | CSF2, CSF1, IFNG |
| h_inflamPathway: Cytokines and | 4 | CSF2, CSF1, IFNG, IL2 |
| Inflammatory Response | | |
| IPR009079: Four- | 5 | LIF, CSF2, CSF1, IFNG, IL2 |
| helical cytokine-like, core | | |
| IPR012351: Four- | 4 | LIF, CSF2, IFNG, IL2 |
| helical cytokine, core | | |
| Growth factor | 7 | LIF, GMFB, CSF2, CD320, CSF1, GFER, IL2 |
| GO: 0005125~cytokine | 11 | LIF, CSF2, IL16, TNFSF13B, FAM3C, CSF1, IFNG, CMTM7, CMTM3, CCL4, |
| activity | | IL2 |
| GO: 0008083~growth factor activity | 8 | LIF, GMFB, CSF2, CD320, CSF1, GFER, NENF, IL2 |
| Enrichment Score: | | |
| 0.1745910687704443 | | |
| domain: LIM zinc-binding 4 | 3 | ABLIM1, LPXN, LIMS1 |
| domain: LIM zinc-binding 3 | 4 | ABLIM1, LPXN, LIMS1, ZYX |
| LIM domain | 7 | ABLIM1, LIMA1, LPXN, LIMS1, LASP1, CSRP1, ZYX |
| domain: LIM zinc-binding 1 | 5 | ABLIM1, LPXN, LIMS1, CSRP1, ZYX |
| domain: LIM zinc-binding 2 | 5 | ABLIM1, LPXN, LIMS1, CSRP1, ZYX |
| SM00132: LIM | 7 | ABLIM1, LIMA1, LPXN, LIMS1, LASP1, CSRP1, ZYX |
| IPR001781: Zinc finger, LIM-type | 7 | ABLIM1, LIMA1, LPXN, LIMS1, LASP1, CSRP1, ZYX |
| Enrichment Score: | | |
| 0.16053907483471863 | | |
| GO: 0051603~proteolysis involved in | 7 | PSMB10, CTSL, LONP1, CASP8, IDE, CTSA, CTSC |
| cellular protein catabolic process | | |
| GO: 0008234~cysteine-type | 5 | CTSL, ATG4B, CASP7, CASP8, CTSC |
| peptidase activity | | |
| Zymogen | 11 | PSMB10, CTSL, TPP1, CASP7, CASP8, HEXB, CTSA, CTSC, GZMB, PCSK7, |
| | | FURIN |
| Enrichment Score: | | |
| 0.15875242349997273 | | |
| domain: EF-hand 5 | 5 | PEF1, CAPNS1, NIN, SDF4, RCN2 |
| calcium-binding | 13 | NUCB1, EFHD2, PEF1, CAPNS1, ACTN4, MCFD2, NUCB2, RHOT1, RHOT2, |
| region: 2 | | CETN2, CHP1, SDF4, RCN2 |
| domain: EF-hand 4 | 7 | PEF1, CAPNS1, NIN, CETN2, CHP1, SDF4, RCN2 |
| IPR011992: EF- | 30 | S100A4, MCL1, REPS1, SPOCK2, UTRN, CETN2, ZZEF1, EFHD2, STAT6, |
| hand-like domain | | PEF1, EHD1, SDF4, EHD4, TBC1D9B, SYNRG, CAPNS1, ACTN4, NIN, CBL, |
| | | S100A11, S100A10, CHP1, NUCB1, CBLB, DEF6, NUCB2, MCFD2, RHOT1, |
| | | RHOT2, RCN2 |
| calcium-binding | 13 | NUCB1, EFHD2, PEF1, CAPNS1, ACTN4, MCFD2, NUCB2, RHOT1, RHOT2, |
| region: 1 | | CETN2, CHP1, SDF4, RCN2 |
| IPR018247: EF- | 17 | S100A4, CAPNS1, ACTN4, REPS1, S100A11, CETN2, NUCB1, PEF1, |
| Hand 1, calcium-binding site | | GNPTAB, NUCB2, MCFD2, RHOT1, RHOT2, EHD1, SDF4, RCN2, EHD4 |
| IPR002048: EF-hand | 22 | S100A4, CAPNS1, ACTN4, NIN, REPS1, S100A11, CHP1, CETN2, ZZEF1, |
| domain | | EFHD2, NUCB1, PEF1, GNPTAB, NUCB2, MCFD2, RHOT1, RHOT2, EHD1, |
| | | SDF4, RCN2, TBC1D9B, EHD4 |
| domain: EF-hand 2 | 16 | S100A4, CAPNS1, NIN, ACTN4, S100A11, CHP1, CETN2, EFHD2, NUCB1, |
| | | PEF1, MCFD2, NUCB2, RHOT1, RHOT2, SDF4, RCN2 |
| SM00054: EFh | 12 | S100A4, EFHD2, PEF1, ACTN4, NUCB2, RHOT1, S100A11, CETN2, CHP1, |
| | | ZZEF1, SDF4, RCN2 |
| domain: EF-hand 1 | 15 | S100A4, NIN, ACTN4, S100A11, CHP1, CETN2, EFHD2, NUCB1, PEF1, |
| | | MCFD2, NUCB2, RHOT1, RHOT2, SDF4, RCN2 |
| domain: EF-hand 3 | 7 | PEF1, CAPNS1, NIN, CETN2, CHP1, SDF4, RCN2 |
| GO: 0005509-calcium | 46 | S100A4, PGS1, ME2, SPOCK2, REPS1, MGMT, CLSTN1, NELL2, CETN2, |
| ion binding | | ZZEF1, EFHD2, PEF1, CD93, GNPTAB, PPP3CB, RUNX1, EHD1, SDF4, |
| | | EHD4, TBC1D9B, CAPNS1, NIN, ACTN4, C2CD5, SYT11, CBL, ANXA1, |
| | | S100A11, S100A10, CHP1, STIM1, ANXA5, ANXA2, ITPR2, NUCB1, |
| | | PLSCR1, CBLB, BNIP2, ATP2C1, MCFD2, NUCB2, RHOT1, CPNE1, RHOT2, |
| | | SYTL3, RCN2 |
| Calcium | 49 | S100A4, ORAIl, SPOCK2, REPS1, ITGAE, CLSTN1, NELL2, UTRN, |
| | | TMEM63A, CETN2, OGDH, ITGB1, EFHD2, PEF1, ATP2B4, GNPTAB, TPP1, |
| | | SNTB1, ENTPD6, CERK, EHD1, SDF4, EHD4, CAPNS1, ACTN4, C2CD5, |
| | | SYT11, CBL, ANXA1, S100A11, CHP1, STIM1, ANXA5, FURIN, ANXA2, |
| | | PRKCB, ITPR2, NUCB1, PLSCR1, CBLB, HSPB11, CAMK4, ATP2C1, |
| | | MCFD2, NUCB2, RHOT1, RHOT2, TMCO1, RCN2 |
| Enrichment Score: | | |
| 0.1459899659776129 | | |
| GO: 1904355~positive regulation of | 3 | MAPK1, MAPKAPK5, MAP2K7 |
| telomere capping | | |
| GO: 0032212~positive regulation of | 4 | MAPK1, MAPKAPK5, MAP2K7, ATM |
| telomere maintenance via telomerase | | |
| GO: 0051973~positive regulation of | 3 | MAPK1, MAPKAPK5, MAP2K7 |
| telomerase activity | | |
| Enrichment Score: | | |
| 0.14095903672022314 | | |
| hsa05332: Graft-versus-host disease | 5 | IFNG, GZMB, HLA-DPA1, HLA-DPB1, IL2 |
| hsa05330: Allograft rejection | 5 | IFNG, GZMB, HLA-DPA1, HLA-DPB1, IL2 |
| hsa04672: Intestinal immune network | 6 | TNFSF13B, CXCR4, HLA-DPA1, HLA-DPB1, MAP3K14, IL2 |
| for IgA production | | |
| hsa04940: Type I diabetes mellitus | 5 | IFNG, GZMB, HLA-DPA1, HLA-DPB1, IL2 |
| hsa05320: Autoimmune thyroid disease | 5 | CTLA4, GZMB, HLA-DPA1, HLA-DPB1, IL2 |
| hsa05322: Systemic lupus erythematosus | 6 | HIST4H4, ACTN4, IFNG, H3F3A, HLA-DPA1, HLA-DPB1 |
| Enrichment Score: | | |
| 0.1385599701644631 | | |
| hsa00330: Arginine and proline | 6 | CNDP2, SAT2, AGMAT, SMS, ALDH3A2, ALDH9A1 |
| metabolism | | |
| hsa00410: beta-Alanine metabolism | 4 | CNDP2, SMS, ALDH3A2, ALDH9A1 |
| hsa00340: Histidine metabolism | 3 | CNDP2, ALDH3A2, ALDH9A1 |
| Enrichment Score: | | |
| 0.13496059793813844 | | |
| 15.T-cell_polarization- | 5 | CXCR4, CCR4, IFNG, CXCR3, TSN |
| chemokine_receptors | | |
| IPR000355: Chemokine receptor family | 3 | CXCR4, CCR4, CXCR3 |
| 14.chemokine_receptor-ligand | 3 | CXCR4, CCR4, CXCR3 |
| GO: 0070098~chemokine-mediated | 7 | CCL3, CXCR4, CCR4, CXCR3, XCL1, CCL4, XCL2 |
| signaling pathway | | |
| IPR000276: G protein-coupled | 9 | P2RY8, P2RY10, RABGAP1, HRH2, CXCR4, LPAR6, CCR4, LHCGR, CXCR3 |
| receptor, rhodopsin-like | | |
| IPR017452: GPCR, | 9 | P2RY8, P2RY10, RABGAP1, HRH2, CXCR4, LPAR6, CCR4, LHCGR, CXCR3 |
| rhodopsin-like, 7TM | | |
| Enrichment Score: | | |
| 0.12648159925240066 | | |
| domain: SAM | 9 | SAMD8, L3MBTL3, SAMD9, STIM1, SAMHD1, DGKH, ARAP2, SAMD9L, |
| | | SASH3 |
| SM00454: SAM | 9 | SAMD8, ANKS1A, L3MBTL3, SAMD9, STIM1, SAMHD1, DGKH, ARAP2, |
| | | SASH3 |
| IPR001660: Sterile | 10 | SAMD8, ANKS1A, L3MBTL3, SAMD9, STIM1, SAMHD1, DGKH, ARAP2, |
| alpha motif domain | | SAMD9L, SASH3 |
| IPR013761: Sterile | 11 | SAMD8, ETV7, ANKS1A, L3MBTL3, SAMD9, STIM1, SAMHD1, DGKH, |
| alpha motif/pointed domain | | ARAP2, SAMD9L, SASH3 |
| Enrichment Score: | | |
| 0.12568460664505718 | | |
| SM00513: SAP | 3 | PIAS4, PIAS1, SAFB2 |
| domain.SAP | 3 | PIAS4, PIAS1, SAFB2 |
| IPR003034: SAP | 3 | PIAS4, PIAS1, SAFB2 |
| domain | | |
| Enrichment Score: | | |
| 0.12346097305495686 | | |
| domain: C2 | 9 | C2CD5, PIK3C2A, PKN2, PRKCH, WWC2, SMURF2, PRKCD, RASA1, |
| | | PRKCB |
| IPR000008: C2 | 17 | CEP120, C2CD5, PIK3C2A, SYT11, PKN2, WWC2, PRKCH, PTEN, PRKCD, |
| calcium-dependent | | PRKCB, GAK, UNC13D, CPNE1, SMURF2, SYTL3, RASA1, RASA2 |
| membrane targeting | | |
| SM00239: C2 | 12 | UNC13D, C2CD5, PIK3C2A, SYT11, PKN2, CPNE1, PRKCH, SMURF2, |
| | | SYTL3, RASA1, RASA2, PRKCB |
| Enrichment Score: | | |
| 0.1227848794008889 | | |
| repeat: 2-1 | 4 | HNRNPH2, PRRC2A, CSTF2T, COIL |
| repeat: 2-2 | 4 | HNRNPH2, PRRC2A, CSTF2T, COIL |
| repeat: 1-1 | 4 | HNRNPH2, PRRC2A, CSTF2T, COIL |
| repeat: 1-2 | 3 | HNRNPH2, PRRC2A, COIL |
| Enrichment Score: | | |
| 0.1141619803729114 | | |
| SM00253: SOCS | 3 | WSB1, SOCS3, SOCS1 |
| domain.SOCS box | 4 | WSB1, SOCS3, SPSB3, SOCS1 |
| SM00969: SM00969 | 4 | WSB1, SOCS3, SPSB3, SOCS1 |
| IPR001496: SOCS | 4 | WSB1, SOCS3, SPSB3, SOCS1 |
| protein, C-terminal | | |
| Enrichment Score: | | |
| 0.1035300297726019 | | |
| GO: 0050660~flavin | 8 | SDHA, IVD, AIFM1, GFER, TXNRD1, DUS1L, ETFA, DUS3L |
| adenine dinucleotide binding | | |
| Flavoprotein | 12 | SDHA, SQRDL, IVD, AlFM1, NDUFA9, PYROXD1, GFER, TXNRD1, |
| | | NDUFA10, DUS1L, ETFA, DUS3L |
| nucleotide phosphate-binding | 6 | SDHA, TXNDC12, IVD, AIFM1, TXNRD1, ETFA |
| region: FAD | | |
| FAD | 10 | SDHA, SQRDL, IVD, AlFM1, NDUFA9, PYROXD1, GFER, TXNRD1, |
| | | NDUFA10, ETFA |
| IPR023753: Pyridine nucleotide- | 5 | SDHA, SQRDL, AIFM1, PYROXD1, TXNRD1 |
| disulphide oxidoreductase, FAD/ | | |
| NAD(P)-binding domain | | |
| Enrichment Score: | | |
| 0.09911308677931861 | | |
| IPR001202: WW domain | 6 | UTRN, FNBP4, WWC2, IQGAP2, SMURF2, CEP164 |
| SM00456: WW | 5 | UTRN, FNBP4, WWC2, SMURF2, CEP164 |
| domain: WW 2 | 3 | FNBP4, WWC2, SMURF2 |
| domain: WW 1 | 3 | FNBP4, WWC2, SMURF2 |
| Enrichment Score: | | |
| 0.09869704499668493 | | |
| IPR014352: FERM/acyl-CoA-binding | 6 | ECI2, FRMD8, KRIT1, FRMD4B, MSN, ACBD5 |
| protein, 3-helical bundle | | |
| domain: FERM | 5 | FRMD8, KRIT1, FRMD4B, JAK2, MSN |
| SM00295: B41 | 5 | FRMD8, KRIT1, FRMD4B, JAK2, MSN |
| IPR000299: FERM domain | 5 | FRMD8, KRIT1, FRMD4B, JAK2, MSN |
| IPR019749: Band 4.1 domain | 5 | FRMD8, KRIT1, FRMD4B, JAK2, MSN |
| IPR019748: FERM central domain | 5 | FRMD8, KRIT1, FRMD4B, JAK2, MSN |
| Enrichment Score: | | |
| 0.09404515348769645 | | |
| GO: 0004623~phospholipase A2 activity | 5 | RARRES3, PNPLA8, ABHD3, PAFAH1B1, PAFAH1B2 |
| GO: 0016042~lipid catabolic process | 7 | PLD3, RARRES3, TBL1XR1, DDHD1, PAFAH1B1, PAFAH1B2, IAH1 |
| Lipid degradation | 8 | PLD3, RARRES3, PNPLA8, DDHD1, ABHD2, PAFAH1B1, PAFAH1B2, IAH1 |
| hsa00565: Ether lipid metabolism | 3 | PLD3, PAFAH1B1, PAFAH1B2 |
| Enrichment Score: | | |
| 0.09224012967461757 | | |
| GO: 0006418~tRNA | 5 | EEF1E1, FARS2, FARSB, WARS2, YARS2 |
| aminoacylation for protein translation | | |
| Aminoacyl-tRNA synthetase | 4 | FARS2, FARSB, WARS2, YARS2 |
| hsa00970: Aminoacy | 5 | FARS2, FARSB, WARS2, YARS2, MTFMT |
| 1-tRNA biosynthesis | | |
| Enrichment Score: | | |
| 0.09104106578512094 | | |
| domain: BACK | 4 | KLHL9, KEAP1, KLHL24, IVNS1ABP |
| IPR015916: Galactose | 4 | KLHL9, KEAP1, KLHL24, IVNS1ABP |
| oxidase, beta-propeller | | |
| GO: 0031463~Cul3- | 8 | CUL3, KBTBD2, BACH2, KLHL9, KEAP1, KLHL24, KCTD2, SPOP |
| RING ubiquitin ligase complex | | |
| IPR017096: Kelch-like protein, | 5 | KBTBD2, KLHL9, KEAP1, KLHL24, IVNS1ABP |
| gigaxonin | | |
| PIR5F037037: kelch-like protein, | 5 | KBTBD2, KLHL9, KEAP1, KLHL24, IVNS1ABP |
| gigaxonin type | | |
| SM00875: SM00875 | 6 | KBTBD2, KLHL9, BTBD7, KEAP1, KLHL24, IVNS1ABP |
| IPR011705: BTB/Kelch-associated | 6 | KBTBD2, KLHL9, BTBD7, KEAP1, KLHL24, IVNS1ABP |
| repeat: Kelch 6 | 5 | MKLN1, KLHL9, KEAP1, KLHL24, IVNS1ABP |
| repeat: Kelch 5 | 6 | MKLN1, KBTBD, KLHL9, KEAP1, KLHL24, IVNS1ABP |
| IPR006652: Kelch repeat type 1 | 6 | MKLN1, KBTBD2, KLHL9, KEAP1, KLHL24, IVNS1ABP |
| repeat: Kelch 4 | 6 | MKLN1, KBTBD2, KLHL9, KEAP1, KLHL24, IVNS1ABP |
| SM00612: Kelch | 5 | KBTBD2, KLHL9, KEAP1, KLHL24, IVNS1ABP |
| Kelch repeat | 6 | MKLN1, KBTBD2, KLHL9, KEAP1, KLHL24, IVNS1ABP |
| repeat: Kelch 2 | 6 | MKLN1, KBTBD2, KLHL9, KEAP1, KLHL24, IVNS1ABP |
| repeat: Kelch 3 | 6 | MKLN1, KBTBD2, KLHL9, KEAP1, KLHL24, IVNS1ABP |
| repeat: Kelch 1 | 6 | MKLN1, KBTBD2, KLHL9, KEAP1, KLHL24, IVNS1ABP |
| IPR015915: Kelch-type beta propeller | 3 | MKLN1, KBTBD2, KEAP1 |
| Enrichment Score: | | |
| 0.09096561156622553 | | |
| GO: 0008037~cell recognition | 4 | TIGIT, CD5, CD200, CD226 |
| GO: 0050839~cell adhesion | 5 | TIGIT, MSN, CD200, ITGB1, CD226 |
| molecule binding | | |
| GO: 0007157~heterophilic cell-cell | 4 | TIGIT, ICAM1, CD200, CD226 |
| adhesion via plasma membrane | | |
| cell adhesion molecules | | |
| GO: 0007156~homophilic cell | 7 | CD84, TIGIT, ME2, CLSTN1, CD200, ITGB1, CD226 |
| adhesion via plasma membrane | | |
| adhesion molecules | | |
| Enrichment Score: | | |
| 0.08637800045583548 | | |
| GO: 0005544~calcium-dependent | 7 | C2CD5, SYT11, ANXA1, CPNE1, SYTL3, ANXA5, ANXA2 |
| phospholipid binding | | |
| SM00239: C2 | 12 | UNC13D, C2CD5, PIK3C2A, SYT11, PKN2, CPNE1, PRKCH, SMURF2, |
| | | SYTL3, RASA1, RASA2, PRKCB |
| domain.C2 2 | 5 | UNC13D, SYT11, CPNE1, SYTL3, RASA2 |
| domain.C2 1 | 5 | UNC13D, SYT11, CPNE1, SYTL3, RASA2 |
| Enrichment Score: | | |
| 0.08611979646447755 | | |
| IPR001751: S100/ | 3 | S100A4, S100A11, S100A10 |
| Calbindin-D9k, conserved site | | |
| SM01394: SM01394 | 3 | S100A4, S100A11, S100A10 |
| IPR013787: S100/CaBP-9k-type, | 3 | S100A4, S100A11, S100A10 |
| calcium binding, subdomain | | |
| Enrichment Score: | | |
| 0.08562305074820027 | | |
| GO: 0034199~activation of protein | 3 | PRKAR2A, ADCY7, PRKACB |
| kinase A activity | | |
| GO: 0003091~renal water homeostasis | 3 | PRKAR2A, ADCY7, PRKACB |
| GO: 0071377~cellular response to | 3 | PRKAR2A, ADCY7, PRKACB |
| glucagon stimulus | | |
| Enrichment Score: | | |
| 0.0850459219054058 | | |
| DNA-binding region: ETS | 3 | ETV7, ELF2, ELK3 |
| SM00413: ETS | 3 | ETV7, ELF2, ELK3 |
| IPR000418: Ets domain | 3 | ETV7, ELF2, ELK3 |
| Enrichment Score: | | |
| 0.08339481617003926 | | |
| GO: 0004715~non-membrane spanning | 7 | DYRK1A, ZAP70, RIPK2, JAK2, ABL2, PRKCD, MATK |
| protein tyrosine kinase activity | | |
| IPR001245: Serine- | 15 | IRAK1, RYK, DSTYK, RAF1, GAK, IRAK4, RIPK1, ARAF, ILK, ZAP70, |
| threonine/tyrosine-protein kinase | | RIPK2, JAK2, ABL2, MAP3K13, MATK |
| catalytic domain | | |
| Tyrosine-protein | 11 | CLK2, RYK, DYRK1A, CLK4, MAP2K4, ZAP70, DSTYK, JAK2, MAP2K7, |
| kinase | | ABL2, MATK |
| GO: 0031234~extrinsic component of | 7 | KRAS, RGS1, TIAM1, ZAP70, JAK2, ABL2, MATK |
| cytoplasmic side of plasma membrane | | |
| GO: 0038083~peptid | 4 | ZAP70, JAK2, ABL2, MATK |
| yl-tyrosine autophosphorylation | | |
| GO: 0004713~protein | 13 | CSF2, ZMYM2, CLK2, RYK, DYRK1A, CLK4, MAP2K4, ZAP70, DSTYK, |
| tyrosine kinase activity | | JAK2, MAP2K7, ABL2, MATK |
| GO: 0018108~peptid | 15 | CSF2, ZMYM2, RYK, MAP2K4, TRIM27, DSTYK, PRKCD, CLK2, CLK4, |
| yl-tyrosine phosphorylation | | DYRK1A, ZAP70, RIPK2, JAK2, MAP2K7, ABL2 |
| GO: 0007169~transmembrane | 7 | CD8A, CD8B, CSF1, ZAP70, RAPGEF1, ABL2, MATK |
| receptor protein tyrosine kinase | | |
| signaling pathway | | |
| SM00219: TyrKc | 5 | RYK, ZAP70, JAK2, ABL2, MATK |
| IPR020635: Tyrosine-protein kinase, | 5 | RYK, ZAP70, JAK2, ABL2, MATK |
| catalytic domain | | |
| IPR008266: Tyrosine-protein kinase, | 5 | RYK, ZAP70, JAK2, ABL2, MATK |
| active site | | |
| Enrichment Score: | | |
| 0.07308177427976495 | | |
| GO: 0004553~hydrolase activity, | 4 | CHID1, GLA, HEXB, HEXDC |
| hydrolyzing O-glycosyl compounds | | |
| IPR017853: Glycoside hydrolase, | 5 | CHID1, GLA, HEXB, MGEA5, HEXDC |
| superfamily | | |
| Glycosidase | 7 | GLA, NEIL2, HEXB, MGEA5, MOGS, OGG1, HEXDC |
| IPR013781: Glycoside hydrolase, | 3 | CHID1, HEXB, HEXDC |
| catalytic domain | | |
| Enrichment Score: | | |
| 0.07228030757809481 | | |
| repeat: Solcar 3 | 5 | SLC25A32, SLC25A38, SLC25A28, SLC25A16, SLC25A53 |
| repeat: Solcar 1 | 5 | SLC25A32, SLC25A38, SLC25A28, SLC25A16, SLC25A53 |
| repeat: Solcar 2 | 5 | SLC25A32, SLC25A38, SLC25A28, SLC25A16, SLC25A53 |
| IPR023395: Mitochondrial | 5 | SLC25A32, SLC25A38, SLC25A28, SLC25A16, SLC25A53 |
| carrier domain | | |
| IPR018108: Mitochondrial | 5 | SLC25A32, SLC25A38, SLC25A28, SLC25A16, SLC25A53 |
| substrate/solute carrier | | |
| Enrichment Score: | | |
| 0.07048632895684756 | | |
| Viral nucleoprotein | 3 | EFTUD2, LARP1B, HNRNPLL |
| GO: 0019013~viral nucleocapsid | 3 | EFTUD2, LARP1B, HNRNPLL |
| Virion | 4 | ERVK13-1, EFTUD2, LARP1B, HNRNPLL |
| Enrichment Score: | | |
| 0.07022853299749594 | | |
| zinc finger region: FYVE-type | 4 | PLEKHF2, HGS, EEA1, RFFL |
| SM00064: FYVE | 3 | PLEKHF2, HGS, EEA1 |
| IPR000306: Zinc | 3 | PLEKHF2, HGS, EEA1 |
| finger, FYVE-type | | |
| IPR017455: Zinc | 3 | PLEKHF2, HGS, EEA1 |
| finger, FYVE-related | | |
| Enrichment Score: | | |
| 0.06811532390904307 | | |
| zinc finger region: | 11 | ZNF43, ZNF529, ZNF44, ZNF28, ZNF121, ZNF675, ZNF766, ATMIN, ZNF586, |
| C2H2-type 2; degenerate | | ZBTB38, ZNF37A |
| zinc finger region: | 5 | ZNF43, ZNF28, ZNF131, ZNF675, ZNF493 |
| C2H2-type 4; degenerate | | |
| zinc finger region: | 4 | ZNF43, ZNF721, ZNF268, ZNF493 |
| C2H2-type 22 | | |
| zinc finger | 4 | ZNF43, ZNF268, ZNF780B, ZNF493 |
| region: C2H2-type 21 | | |
| GO: 0003676~nucleic | 112 | RALY, ZNF583, CNOT8, RNASEH1, ZNF638, SART3, DDX23, ZFP90, TIA1, |
| acid binding | | TARDBP, DHX34, RBM10, ZNF101, ZNF43, R3HCC1, ZNF44, ZNF644, |
| | | ZNF814, ZNF7, DHX29, ZNF587, ZNF586, ZNF430, SETD1A, ZNF131, RAC1, |
| | | PPIL4, DDX42, ZNF529, IKZF2, KIAA1586, KLF13, SREK1, ZNF121, |
| | | ZBTB40, MTHFSD, ENDOD1, SAFB2, JAZFL PPRC1, HIVEP1, RNPC3, |
| | | POP? |
| | | HNRNPLL, ZEB1, ZBTB38, ZNF148, AEN, RBAK-RBAKDN, ZNF721, |
| | | ZNF720, TSEN2, ZCCHC6, ZNF493, ZCCHC7, ZCCHC10, RBM42, ZFX, |
| | | SPEN, TTF2, RECQL, POGK, RBMX2, ZNF746, CPSF4, ZNF740, ZNF276, |
| | | ZNF275, ZNF274, RBM33, ZBTB10, ZBTB11, CBLL1, ERI3, ZNF780B, |
| | | ZNF780A, SF3B4, SF3B3, CHD1L, DDX19A, REX01, AGO2, DHX16, |
| | | ZSCAN25, MSI2, THAP1, ZNF268, RBM28, EHD4, TTC14, RBM23, ZNF28, |
| | | ALYREF, TRIM27, RAF1, GPANK1, SAMHD1, SF3A2, ZNF664, ZNF672, |
| | | POLDIP3, RBM19, ZNF764, ZNF766, RBM15 |
| zinc finger region: | 4 | ZNF43, ZNF268, ZNF780B, ZNF493 |
| C2H2-type 20 | | |
| zinc finger region: | 12 | ZNF529, ZNF44, ZNF28, ZNF121, ZNF800, ZNF675, ZNF746, ZNF721, |
| C2H2-type 1; degenerate | | ZNF766, ZNF493, ZNF586, ZNF37A |
| zinc finger | 8 | ZNF43, ZNF44, ZNF28, ZNF721, ZNF268, ZNF780B, ZNF780A, ZNF493 |
| region: C2H2-type 17 | | |
| zinc finger region: | 6 | ZNF43, ZNF28, ZNF721, ZNF268, ZNF780B, ZNF493 |
| C2H2-type 18 | | |
| zinc finger region: | 5 | ZNF43, ZNF721, ZNF268, ZNF780B, ZNF493 |
| C2H2-type 19 | | |
| zinc finger region: | 11 | ZNF43, ZNF44, ZNF534, ZNF28, ZNF675, ZNF7, ZNF721, ZNF268, ZNF780B, |
| C2H2-type 15 | | ZNF780A, ZNF493 |
| zinc finger region: | 8 | ZNF43, ZNF44, ZNF28, ZNF721, ZNF268, ZNF780B, ZNF780A, ZNF493 |
| C2H2-type 16 | | |
| zinc finger region: | 12 | ZNF43, ZNF44, ZNF672, ZNF534, ZNF28, ZNF675, ZNF7, ZNF721, ZNF268, |
| C2H2-type 14 | | ZNF780B, ZNF780A, ZNF493 |
| zinc finger | 49 | ZNF276, ZNF275, ZNF430, ZNF274, ZNF292, ZNF534, ZNF583, ZBTB10, |
| region: C2H2-type 1 | | ZBTB11, ZNF131, ZNF511, CTCF, ZKSCAN1, ZEB1, ZNF780B, ZNF780A, |
| | | ZBTB38, ZNF148, ZFP90, ZSCAN25, ZNF394, ZNF268, ZNF101, PLAGL2, |
| | | IKZF5, ZNF43, EGR1, IKZF2, ZNF644, KLF13, KLF10, ZHX1, ZFX, ZBTB40, |
| | | ZNF7, ATMIN, TRERF1, ZNF664, ZNF672, SP1, ZNF277, JAZFL HIVEP2, |
| | | PRDM2, ZBTB2, HIVEP1, ZNF764, ZNF587, ZNF740 |
| zinc finger | 28 | ZNF275, ZNF430, ZNF583, ZNF534, ZBTB11, ZNF675, CTCF, ZNF780B, |
| region: C2H2-type | | ZNF780A, ZBTB38, ZFP90, ZNF721, ZNF268, ZNF493, ZNF101, ZNF43, |
| 10 | | ZNF529, ZNF44, ZNF28, ZNF121, ZFX, ZBTB40, ZNF7, ZNF37A, ZNF672, |
| | | ZNF587, ZNF766, ZNF586 |
| zinc finger | 31 | ZNF275, ZNF292, ZNF430, ZNF583, ZNF534, ZBTB11, ZNF675, CTCF, |
| region: C2H2-type 9 | | ZNF780B, ZNF780A, ZBTB38, ZFP90, ZNF721, ZNF268, ZNF493, ZNF101, |
| | | ZNF43, ZNF529, ZNF44, ZNF28, POGZ, ZNF121, ZFX, ZBTB40, ZNF7, |
| | | ZNF37A, ZNF664, ZNF672, ZNF587, ZNF766, ZNF586 |
| zinc finger | 23 | ZNF43, ZNF275, ZNF529, ZNF430, ZNF44, ZNF583, ZNF534, ZNF28, |
| region: C2H2-type | | ZNF121, ZBTB11, ZFX, ZNF675, ZNF7, ZNF780B, ZNF780A, ZNF37A, |
| 11 | | ZNF672, ZFP90, ZNF721, ZNF268, ZNF587, ZNF493, ZNF586 |
| zinc finger | 19 | ZNF43, ZNF44, ZNF583, ZNF534, ZNF28, ZBTB11, ZFX, ZBTB40, ZNF675, |
| region: C2H2-type | | ZNF7, ZNF780B, ZNF780A, ZNF37A, ZNF672, ZFP90, ZNF721, ZNF268, |
| 12 | | ZNF587, ZNF493 |
| zinc finger | 52 | ZNF292, ZNF583, ZNF534, ZKSCAN1, CTCF, ZEB1, ZNF148, ZFP90, |
| region: C2H2-type 2 | | ZNF394, ZNF721, ZNF493, ZNF101, EGR1, ZNF44, ZNF644, POGZ, ZHX1, |
| | | ZFX, ZNF7, TRERF1, ZNF277, PRDM2, ZNF746, ZNF587, ZNF740, ZNF276, |
| | | ZNF275, ZNF274, ZNF430, ZBTB10, ZBTB11, ZNF131, ZNF800, ZNF511, |
| | | ZNF780B, ZNF780A, ZSCAN25, ZNF268, PLAGL2, IKZF5, IKZF2, KLF13, |
| | | KLF10, ZBTB40, ZNF664, ZNF672, SP1, JAZFL HIVEP2, HIVEP1, ZBTB2, |
| | | ZNF764 |
| zinc finger | 41 | ZNF275, ZNF430, ZNF292, ZNF583, ZNF534, ZBTB11, ZNF131, ZNF800, |
| region: C2H2-type 6 | | ZNF675, ZKSCAN1, CTCF, ZEB1, ZNF780B, ZNF780A, ZBTB38, ZFP90, |
| | | ZSCAN25, ZNF721, ZNF394, ZNF268, ZNF101, PLAGL2, ZNF43, ZNF529, |
| | | ZNF44, IKZF2, ZNF644, ZNF28, POGZ, ZNF121, ZFX, ZBTB40, ZNF7, |
| | | ZNF664, ZNF37A, ZNF672, PRDM2, ZNF764, ZNF587, ZNF766, ZNF586 |
| IPRO07087: Zinc | 72 | ZNF292, ZNF534, ZNF583, ZNF675, CTCF, ZKSCAN1, ZEB1, ZNF638, |
| finger, C2H2 | | ZBTB38, BRPF1, ZNF148, ZFP90, ZNF106, ZNF721, ZNF394, RBM10, |
| | | ZNF493, ZNF101, ZNF43, EGR1, ZNF44, ZNF644, POGZ, ZFX, ZHX1, |
| | | ZNF814, APTX, ZNF7, TRERF1, ZNF37A, ZNF277, PRDM2, ZNF746, |
| | | ZNF587, ZNF740, ZNF586, ZNF276, DPF2, ZNF275, ZNF430, ZNF274, |
| | | ZBTB10, ZBTB11, ZNF131, ZNF800, ZNF511, DUSP12, EEA1, ZNF780B, |
| | | ZNF780A, ZSCAN25, ZNF268, PLAGL2, IKZF5, ZNF529, IKZF2, ZNF28, |
| | | KLF13, ZNF121, KLF10, ZBTB40, FOXP3, ATMIN, ZNF664, ZNF672, SP1, |
| | | JAZFL, HIVEP2, HIVEP1, ZBTB2, ZNF764, ZNF766 |
| SM00355: ZnF_C2H | 66 | ZNF292, ZNF534, ZNF583, ZNF675, CTCF, ZKSCAN1, ZEB1, ZNF638, |
| 2 | | ZBTB38, ZNF148, ZFP90, ZNF106, ZNF721, ZNF394, ZN1F493, ZNF101, |
| | | ZNF43, EGR1, ZNF44, ZNF644, POGZ, ZFX, ZHX1, ZNF814, ZNF7, TRERF1, |
| | | ZNF37A, ZNF277, PRDM2, ZNF746, ZNF587, ZNF740, ZNF586, ZNF276, |
| | | DPF2, ZNF275, ZNF430, ZNF274, ZBTB10, ZBTB11, ZNF131, ZNF800, |
| | | ZNF511, ZNF780B, ZNF780A, ZSCAN25, ZNF268, PLAGL2, IKZF5, ZNF529, |
| | | IKZF2, ZNF28, KLF13, ZNF121, KLF10, ZBTB40, ATMIN, ZNF664, ZNF672, |
| | | SP1, JAZFL HIVEP2, HIVEP1, ZBTB2, ZNF764, ZNF766 |
| zinc finger | 14 | ZNF43, ZNF44, ZNF534, ZNF28, ZFX, ZNF675, ZNF7, ZNF780B, ZNF780A, |
| region: C2H2-type 13 | | ZNF672, ZFP90, ZNF268, ZNF587, ZNF493 |
| zinc finger | 44 | ZNF276, ZNF275, ZNF430, ZNF274, ZNF292, ZNF583, ZNF534, ZBTB11, |
| region: C2H2-type 5 | | ZNF131, ZNF800, ZNF675, ZKSCAN1, CTCF, ZEB1, ZNF780B, ZNF780A, |
| | | ZBTB38, ZFP90, ZSCAN25, ZNF394, ZNF268, ZNF493, ZNF101, PLAGL2, |
| | | IKZF5, ZNF43, ZNF529, ZNF44, IKZF2, ZNF644, ZNF28, POGZ, ZNF121, |
| | | ZFX, ZBTB40, ZNF7, ZNF664, ZNF37A, PRDM2, HIVEP1, ZNF764, ZNF587, |
| | | ZNF766, ZNF586 |
| domain: KRAB | 25 | ZNF275, ZNF430, ZNF534, ZNF583, ZNF675, ZKSCAN1, ZNF780B, |
| | | ZNF780A, ZFP90, ZNF394, ZNF268, ZNF720, ZNF101, ZNF43, ZNF529, |
| | | ZNF44, ZNF28, ZNF7, ZNF37A, POGK, ZNF764, ZNF746, ZNF587, ZNF766, |
| | | ZNF586 |
| IPR015880: Zinc | 66 | ZNF292, ZNF534, ZNF583, ZNF675, CTCF, ZKSCAN1, ZEB1, ZNF638, |
| finger, C2H2-like | | ZBTB38, ZNF148, ZFP90, ZNF106, ZNF721, ZNF394, ZN1F493, ZNF101, |
| | | ZNF43, EGR1, ZNF44, ZNF644, POGZ, ZFX, ZHX1, ZNF814, ZNF7, TRERF1, |
| | | ZNF37A, ZNF277, PRDM2, ZNF746, ZNF587, ZNF740, ZNF586, ZNF276, |
| | | DPF2, ZNF275, ZNF430, ZNF274, ZBTB10, ZBTB11, ZNF131, ZNF800, |
| | | ZNF511, ZNF780B, ZNF780A, ZSCAN25, ZNF268, PLAGL2, IKZF5, ZNF529, |
| | | IKZF2, ZNF28, KLF13, ZNF121, KLF10, ZBTB40, ATMIN, ZNF664, ZNF672, |
| | | SP1, JAZFL HIVEP2, HIVEP1, ZBTB2, ZNF764, ZNF766 |
| SM00349: KRAB | 29 | ZNF274, ZNF430, ZNF583, ZNF534, ZNF675, ZKSCAN1, ZNF780B, |
| | | ZNF780A, ZFP90, RBAK-RBAKDN, ZNF394, ZNF721, ZNF268, ZNF720, |
| | | ZNF493, ZNF101, ZNF43, ZNF529, ZNF44, ZNF28, ZNF814, ZNF7, ZNF37A, |
| | | POGK, ZNF764, ZNF746, ZNF587, ZNF766, ZNF586 |
| IPR013087: Zinc | 60 | ZNF292, ZNF583, ZNF534, ZNF675, ZKSCAN1, CTCF, ZEB1, ZBTB38, |
| finger C2H2- | | ZNF148, ZFP90, ZNF721, ZNF394, ZNF493, ZNF101, ZNF43, EGR1, ZNF44, |
| type/integrase DNA- | | ZNF644, ZFX, ZNF814, ZNF7, ZNF37A, PRDM2, ZNF746, ZNF587, ZNF740, |
| binding domain | | ZNF586, ZNF276, ZNF275, ZNF430, ZNF274, ZBTB10, ZBTB11, ZNF131, |
| | | ZNF800, ZNF511, CBLL1, ZNF780B, ZNF780A, ZSCAN25, ZNF268, PLAGL2, |
| | | IKZF5, ZNF529, IKZF2, ZNF28, KLF13, ZNF121, KLF10, ZBTB40, FOXP3, |
| | | ZNF664, ZNF672, SP1, JAZFL HIVEP2, HIVEP1, ZBTB2, ZNF764, ZNF766 |
| zinc finger | 50 | ZNF292, ZNF583, ZNF534, ZKSCAN1, CTCF, ZEB1, ZBTB38, ZNF148, |
| region: C2H2-type 3 | | ZFP90, ZNF394, ZNF493, EGR1, ZNF44, ZNF644, POGZ, ZFX, ZNF7, |
| | | TRERF1, ZNF37A, PRDM2, ZNF746, ZNF587, ZNF276, ZNF275, ZNF274, |
| | | ZNF430, ZBTB11, ZNF131, ZNF800, ZNF511, ZNF780B, ZNF780A, |
| | | ZSCAN25, ZNF268, PLAGL2, IKZF5, ZNF529, IKZF2, ZNF28, KLF13, |
| | | ZNF121, KLF10, ZBTB40, ZNF664, ZNF672, SP1, HIVEP2, HIVEP1, ZNF764, |
| | | ZNF766 |
| IPR001909: Krueppe | 30 | ZNF274, ZNF430, ZNF583, ZNF534, ZNF675, ZKSCAN1, ZNF780B, |
| 1-associated box | | ZNF780A, ZFP90, ZSCAN25, RBAK-RBAKDN, ZNF394, ZNF721, ZNF268, |
| | | ZNF720, ZNF493, ZNF101, ZNF43, ZNF529, ZNF44, ZNF28, ZNF814, ZNF7, |
| | | ZNF37A, POGK, ZNF746, ZNF764, ZNF587, ZNF766, ZN1F586 |
| zinc finger | 34 | ZNF275, ZNF430, ZNF292, ZNF583, ZNF534, ZBTB11, ZNF800, ZNF675, |
| region: C2H2-type 7 | | CTCF, ZNF780B, ZNF780A, ZBTB38, ZFP90, ZNF721, ZNF394, ZNF268, |
| | | ZNF101, ZNF43, ZNF529, ZNF44, ZNF644, ZNF28, POGZ, ZNF121, ZFX, |
| | | ZBTB40, ZNF7, ZNF37A, ZNF664, ZNF672, ZNF764, ZN1F587, ZNF766, |
| | | ZNF586 |
| zinc finger | 30 | ZNF275, ZNF292, ZNF430, ZNF583, ZNF534, ZBTB11, ZNF675, CTCF, |
| region: C2H2-type 8 | | ZNF780B, ZNF780A, ZBTB38, ZFP90, ZNF721, ZNF268, ZNF101, ZNF43, |
| | | ZNF529, ZNF44, ZNF28, POGZ, ZNF121, ZFX, ZBTB40, ZNF7, ZNF37A, |
| | | ZNF664, ZNF672, ZNF587, ZNF766, ZNF586 |
| zinc finger | 43 | ZNF276, ZNF275, ZNF430, ZNF274, ZNF292, ZNF583, ZNF534, ZBTB11, |
| region: C2H2-type 4 | | ZNF800, ZKSCAN1, CTCF, ZNF780B, ZNF780A, ZBTB38, ZNF148, ZFP90, |
| | | ZSCAN25, ZNF721, ZNF394, ZNF268, ZNF101, PLAGL2, IKZF5, ZNF529, |
| | | ZNF44, IKZF2, ZNF644, POGZ, ZNF121, ZFX, ZBTB40, ZNF7, ZNF37A, |
| | | ZNF664, ZNF672, HIVEP2, PRDM2, HIVEP1, ZNF746, ZNF764, ZNF587, |
| | | ZNF766, ZNF586 |
| Enrichment Score: | | |
| 0.06745858636176846 | | |
| short sequence | 3 | IL2RB, IL6ST, IL4R |
| motif: Box 1 motif | | |
| short sequence | 3 | IL2RB, IL6ST, IL4R |
| motif: WSXWS motif | | |
| IPR003961: Fibronectin, | 9 | ATF7IP, IFNAR2, IL2RB, IL6ST, IL4R, LRRN3, IFNGR2, ATF7IP2, IFNAR1 |
| type III | | |
| Enrichment Score: | | |
| 0.06738194626763314 | | |
| domain: Ig-like C1-type | 4 | HLA-DPA1, MR1, HLA-DPB1, TAPBPL |
| region of | 4 | HLA-DPA1, MR1, HLA-DPB1, CRYBB2 |
| interest: Connecting peptide | | |
| GO: 0042605~peptide antigen binding | 3 | HLA-DPA1, MR1, HLA-DPB1 |
| IPR003597: Immuno globulin C1-set | 6 | HLA-DPA1, MR1, HLA-DPB1, TRDC, TAPBPL, IGHM |
| IPR003006: Immuno globulin/major | 5 | HLA-DPA1, MR1, HLA-DPB1, TAPBPL, IGHM |
| histocompatibility complex, | | |
| conserved site | | |
| SM00407: IGc 1 | 5 | HLA-DPA1, MR1, HLA-DPB1, TAPBPL, IGHM |
| IPR011162: MHC classes I/II-like | 3 | HLA-DPA1, MR1, HLA-DPB1 |
| antigen recognition protein | | |
| Enrichment Score: | | |
| 0.0547491368027546 | | |
| IPR023415: Low-density lipoprotein | 4 | DGCR2, CD320, LRP10, LDLRAD4 |
| (LDL) receptor class A, conserved site | | |
| SM00192: LDLa | 4 | DGCR2, CD320, LRP10, LDLRAD4 |
| IPR002172: Low-density lipoprotein | 4 | DGCR2, CD320, LRP10, LDLRAD4 |
| (LDL) receptor class A repeat | | |
| Enrichment Score: | | |
| 0.05398774065645578 | | |
| metal ion-binding | 3 | SYT11, PMF1, PRKCB |
| site: Calcium 3 | | |
| metal ion-binding | 5 | ATP2C1, SYT11, PMF1, FURIN, PRKCB |
| site: Calcium 2 | | |
| metal ion-binding | 4 | SYT11, PMF1, FURIN, PRKCB |
| site: Calcium 1 | | |
| Enrichment Score: | | |
| 0.05304803073201943 | | |
| Thiol protease inhibitor | 4 | XIAP, CARD16, CSTB, BIRC6 |
| GO: 0004869~cysteine-type | 3 | CARD16, CSTB, BIRC6 |
| endopeptidase inhibitor activity | | |
| GO: 0010951~negative regulation of | 8 | SERPINB9, CARD16, SPOCK2, CSTB, SERPINB1, BIRC6, FURIN, APLP2 |
| endopeptidase activity | | |
| Protease inhibitor | 7 | SERPINB9, XIAP, CARD16, CSTB, SERPINB1, BIRC6, APLP2 |
| Serine protease inhibitor | 3 | SERPINB9, SERPINB1, APLP2 |
| GO: 0004867~serine-type endopeptidase | 4 | SERPINB9, SERPINB1, FURIN, APLP2 |
| inhibitor activity | | |
| Enrichment Score: | | |
| 0.04732472305636867 | | |
| GO: 0046983~protein dimerization | 16 | E2F3, E2F4, AIFM1, PEX3, MXI1, ATM, MXD4, SREBF2, PEF1, NCOA1, |
| activity | | NCOA2, HES4, NUP210, GATA3, PPP3CB, FBXW11 |
| DNA-binding | 16 | BACH2, CREBZF, CREB1, MXI1, MXD4, SREBF2, ATF6, ATF5, FOS, |
| region: Basic motif | | NCOA1, NCOA2, HES4, JUN, NFE2L2, NFE2L3, TCF3 |
| SM00353: HLH | 7 | NCOA1, NCOA2, HES4, MXI1, TCF3, MXD4, SREBF2 |
| domain: Helix-loop-helix motif | 7 | NCOA1, NCOA2, HES4, MXI1, TCF3, MXD4, SREBF2 |
| IPR011598: Myc-type, basic helix- | 7 | NCOA1, NCOA2, HES4, MXI1, TCF3, MXD4, SREBF2 |
| loop-helix (bHLH) domain | | |
| Enrichment Score: | | |
| 0.04017008442387107 | | |
| hsa04918: Thyroid hormone synthesis | 9 | GPX1, ADCY7, CREB1, PRKACB, PDIA4, GPX7, TTF2, PRKCB, ITPR2 |
| hsa04925: Aldosterone synthesis and | 9 | ORAIl, CAMK4, ADCY7, CREB1, NR4A1, PRKACB, PRKD3, PRKCB, ITPR2 |
| secretion | | |
| hsa04750: Inflammatory mediator | 10 | ADCY7, MAPK13, PRKCH, MAPK8, PRKACB, PRKCD, PPP1CB, PIK3R1, |
| regulation of TRP channels | | PRKCB, ITPR2 |
| hsa04971: Gastric acid secretion | 5 | ADCY7, HRH2, PRKACB, PRKCB, ITPR2 |
| hsa04970: Salivary secretion | 6 | ATP2B4, ADCY7, PRKACB, VAMP2, PRKCB, ITPR2 |
| hsa04723: Retrograde endocannabinoid | 7 | MAPK1, ADCY7, MAPK13, MAPK8, PRKACB, PRKCB, ITPR2 |
| signaling | | |
| hsa04724: Glutamate rgic synapse | 8 | MAPK1, GRM4, GLUL, ADCY7, PPP3CB, PRKACB, PRKCB, ITPR2 |
| hsa05032: Morphine addiction | 6 | ADCY7, PDE7A, PDE4B, PDE4D, PRKACB, PRKCB |
| hsa04713: Circadian entminment | 6 | MAPK1, FOS, ADCY7, CREB1, PRKACB, PRKCB |
| hsa04020: Calcium | 13 | PHKA2, PPIF, ORAIl, ATP2B4, CAMK4, ADCY7, HRH2, LHCGR, PPP3CB, |
| signaling pathway | | STIM1, PRKACB, PRKCB, ITPR2 |
| hsa04911: Insulin secretion | 5 | ADCY7, CREB1, PRKACB, VAMP2, PRKCB |
| hsa04727: GABAergic synapse | 5 | GABARAPL2, GLUL, ADCY7, PRKACB, PRKCB |
| hsa04972: Pancreatic secretion | 5 | ATP2B4, ADCY7, RAC1, PRKCB, ITPR2 |
| Enrichment Score: | | |
| 0.03512913972098621 | | |
| domain: IQ 1 | 3 | CAMTA2, IQGAP2, MYO9B |
| domain: IQ 2 | 3 | CAMTA2, IQGAP2, MYO9B |
| IPR000048: IQ motif, EF-hand | 3 | CAMTA2, IQGAP2, MYO9B |
| binding site | | |
| Enrichment Score: | | |
| 0.03283703716156655 | | |
| GO: 0035025~positive regulation of Rho | 4 | P2RY8, P2RY10, LPAR6, RAC1 |
| protein signal transduction | | |
| GO: 0051482~positive regulation of | 3 | P2RY8, P2RY10, LPAR6 |
| cytosolic calcium ion concentration | | |
| involved in phospholipase C-activating | | |
| G-protein coupled signaling pathway | | |
| hsa04080: Neuroactive ligand-receptor | 7 | P2RY8, GRM4, TSPO, P2RY10, HRH2, LPAR6, LHCGR |
| interaction | | |
| IPR000276: G protein-coupled | 9 | P2RY8, P2RY10, RABGAP1, HRH2, CXCR4, LPAR6, CCR4, LHCGR, CXCR3 |
| receptor, rhodopsin-like | | |
| G-protein coupled receptor | 10 | P2RY8, GRM4, P2RY10, RABGAP1, HRH2, CXCR4, LPAR6, CCR4, LHCGR, |
| | | CXCR3 |
| IPR017452: GPCR, | 9 | P2RY8, P2RY10, RABGAP1, HRH2, CXCR4, LPAR6, CCR4, LHCGR, CXCR3 |
| rhodopsin-like, 7TM | | |
| GO: 0004930~G-protein coupled | 9 | P2RY8, GRM4, P2RY10, RABGAP1, TM2D1, CXCR4, LPAR6, IGF2R, TPRA1 |
| receptor activity | | |
| Transducer | 11 | GNA13, P2RY8, GRM4, P2RY10, RABGAP1, HRH2, CXCR4, LPAR6, CCR4, |
| | | LHCGR, CXCR3 |
| Enrichment Score: | | |
| 0.015490657362452302 | | |
| domain: Ig-like V- | 13 | TIGIT, BTLA, BSG, CD8A, CD8B, CTLA4, CD79B, TAPBPL, CD200, |
| type | | BTN3A2, LSR, LAG3, PDCD1 |
| Immunoglobulin | 29 | CD8A, CD8B, IL6ST, IGHM, LSR, PDCD1, EMB, MR1, LAG3, ICAM1, |
| domain | | IL18R1, C100RF54, BSG, LRRN3, ICAM3, CTLA4, MALT1, SLAMF7, TIGIT, |
| | | BTLA, CD84, IGSF8, BTN3A1, CD79B, MFAP3, TAPBPL, BTN3A2, CD200, |
| | | CD226 |
| SM00409: IG | 27 | CD8A, CD8B, IGHM, LSR, PDCD1, EMB, LAG3, ICAM1, IL18R1, C10ORF54, |
| | | BSG, LRRN3, ICAM3, CTLA4, MALT1, TIGIT, BTLA, CD84, IGSF8, |
| | | BTN3A1, SP1, CD79B, MFAP3, TAPBPL, CD200, BTN3A2, CD226 |
| IPR003599: Immuno | 27 | CD8A, CD8B, IGHM, LSR, PDCD1, EMB, LAG3, ICAM1, IL18R1, C10ORF54, |
| globulin subtype | | BSG, LRRN3, ICAM3, CTLA4, MALT1, TIGIT, BTLA, CD84, IGSF8, |
| | | BTN3A1, SP1, CD79B, MFAP3, TAPBPL, CD200, BTN3A2, CD226 |
| SM00406: IGv | 8 | BTN3A1, CD8A, CD8B, CTLA4, IGHM, BTN3A2, CD200, PDCD1 |
| IPR013106: Immuno | 16 | C100RF54, CD8A, CD8B, CTLA4, SLAMF7, IGHM, PDCD1, TIGIT, BTN3A1, |
| globulin V-set | | IGSF8, SP1, CD79B, TAPBPL, CD200, CD226, BTN3A2 |
| IPR007110: Immuno | 33 | CD8A, IL6ST, CD8B, TRDC, IGHM, LSR, PDCD1, HLA-DPB1, EMB, MR1, |
| globulin-like domain | | LAG3, ICAM1, IL18R1, C10ORF54, BSG, ICAM3, LRRN3, CTLA4, MALT1, |
| | | SLAMF7, TIGIT, BTLA, CD84, IGSF8, BTN3A1, SP1, CD79B, HLA-DPA1, |
| | | MFAP3, TAPBPL, BTN3A2, CD200, CD226 |
| IPR013783: Immuno | 43 | CD8A, IL6ST, CD8B, TRDC, IGHM, LSR, PDCD1, MYCBP2, IL4R, HLA- |
| globulin-like fold | | DPB1, EMB, MR1, NFATC2, IFNGR2, LAG3, ATF7IP, ICAM1, C100RF54, |
| | | IL18R1, IL2RB, BSG, RELA, ICAM3, LRRN3, CTLA4, MALT1, SLAMF7, |
| | | FLNA, IFNAR1, CD84, TIGIT, BTLA, IFNAR2, BTN3A1, IGSF8, SP1, CD79B, |
| | | HLA-DPA1, MFAP3, TAPBPL, BTN3A2, CD200, CD226 |
| Enrichment Score: | | |
| 0.012624733221056823 | | |
| SM00431: SCAN | 4 | ZNF274, ZSCAN25, ZKSCAN1, ZNF394 |
| domain SCAN box | 4 | ZNF274, ZSCAN25, ZKSCAN1, ZNF394 |
| IPR003309: Transcription regulator | 4 | ZNF274, ZSCAN25, ZKSCAN1, ZNF394 |
| SCAN | | |
| IPR008916: Retrovirus capsid, C- | 4 | ZNF274, ZSCAN25, ZKSCAN1, ZNF394 |
| terminal | | |
| Enrichment Score: | | |
| 0.01039742026464367 | | |
| domain.VWFA | 5 | ITGAE, INTS6, CPNE1, PARP4, ITGB1 |
| IPR002035: von | 5 | ITGAE, INTS6, CPNE1, PARP4, ITGB1 |
| Willebrand factor, type A | | |
| SM00327: VWA | 3 | ITGAE, CPNE1, PARP4 |
| Enrichment Score: | | |
| 0.010098512996503222 | | |
| SM00339: FH | 3 | FOXK2, FOXJ3, FOXP3 |
| DNA-binding region: Fork-head | 3 | FOXK2, FOXJ3, FOXP3 |
| IPR001766: Transcription factor, | 3 | FOXK2, FOXJ3, FOXP3 |
| fork head | | |
| Enrichment Score: | | |
| 0.009453656970068324 | | |
| GO: 0014069~postsynaptic density | 15 | DBNL, LZTS3, DNM3, SYT11, FMR1, RGS19, STRN, DTNBP1, RGS14, PJA2, |
| | | NCOA2, SIPA1L1, SOS1, PDE4B, GOPC |
| Postsynaptic cell | 11 | PJA2, PRR7, LZTS3, SIPA1L1, FMR1, UTRN, CL STN1, GOPC, TMUB1, |
| membrane | | DTNBP1, RGS14 |
| Cell junction | 51 | LZTS3, LIMA1, UTRN, CL STN1, DSTYK, ARHGAP17, ZNRF1, MIT′, |
| | | CXCR4, TIAM1, GOPC, ILK, SNTB1, TMUB1, ZYX, DPP4, PARVG, SYMPK, |
| | | DBNL, ACTN4, FMR1, PKN2, VEZT, GAK, PJA2, SIPA1L1, KRIT1, TBCD, |
| | | ASH1L, TCHP, SDCBP, VAMP2, MPST, LIMS1, ARFGEF2, ITGB1, APBB lIP, |
| | | DTNBP1, PRR7, LPXN, RAB11B, SNAP23, EMB, PLEC, APC, FYB, ICA1, |
| | | SYT11, SNAPIN, RGS14, SYNE2 |
| GO: 0045211~postsynaptic | 13 | LZTS3, FMR1, CL STN1, UTRN, STRN, PIEN, DTNBP1, RGS14, PRR7, PJA2, |
| membrane | | SIPA1L1, GOPC, TMUB1 |
| Synapse | 23 | LZTS3, DBNL, ICA1, SYT11, FMR1, UTRN, CL STN1, SNAPIN, ZNRF1, |
| | | ARFGEF2, DTNBP1, RGS14, PJA2, PRR7, MFF, SIPA1L1, GOPC, RAB11B, |
| | | TMUB1, VAMP2, SNAP23, EMB, MPST |
| GO: 0030054~cell | 32 | LZTS3, OSBP, UTRN, CL STN1, DSTYK, ZNRF1, ARFGEF2, DTNBP1, PRR7, |
| junction | | MFF, CXCR4, GOPC, ILK, RAB11B, TMUB1, EMB, FYB, ICA1, SYT11, |
| | | FMR1, PKN2, SMC5, SNAPIN, RGS14, NRIP1, PJA2, DCP2, GTF2F1, |
| | | SIPA1L1, DDB2, VAMP2, MPST |
| Enrichment Score: | | |
| 0.004733778950614591 | | |
| GO: 0005244~voltage-gated ion channel | 3 | TMEM109, CLIC1, HVCN1 |
| activity | | |
| GO: 0034765~regulation of ion | 3 | TMEM109, CLIC1, HVCN1 |
| transmembrane transport | | |
| Voltage-gated channel | 3 | TMEM109, CLIC1, HVCN1 |
| Enrichment Score: | | |
| 0.0031554828769724845 | | |
| domain: PDZ | 7 | PDZD8, TIAM1, SIPA1L1, GOPC, RAPGEF6, SNTB1, MPP6 |
| SM00228: PDZ | 9 | PDZD8, IL16, TIAM1, SIPA1L1, GOPC, RAPGEF6, SNTB1, SDCBP, MPP6 |
| IPR001478: PDZ domain | 9 | PDZD8, IL16, TIAM1, SIPA1L1, GOPC, RAPGEF6, SNTB1, SDCBP, MPP6 |
| Enrichment Score: | | |
| 0.0017938227712904558 | | |
| repeat: LRR 12 | 6 | NLRC5, SYNE2, LRRC8B, LRRN3, LRRC8D, SHOC2 |
| repeat: LRR 11 | 6 | NLRC5, SYNE2, LRRC8B, LRRN3, LRRC8D, SHOC2 |
| repeat: LRR 10 | 7 | NLRC5, SYNE2, PPP1R7, LRRC8B, LRRN3, LRRC8D, SHOC2 |
| repeat: LRR 13 | 4 | NLRC5, SYNE2, LRRC8B, SHOC2 |
| repeat: LRR 9 | 7 | NLRC5, SYNE2, PPP1R7, LRRC8B, LRRN3, LRRC8D, SHOC2 |
| repeat: LRR 7 | 10 | NLRC5, RSUl, SYNE2, PPP1R7, LRRC8B, LRRC41, LRRN3, LRRC8D, |
| | | LHCGR, SHOC2 |
| repeat: LRR 6 | 12 | NLRC5, RSUl, SYNE2, PPP1R7, LRRC8B, LRRC41, LRRN3, LRRC8D, |
| | | LHCGR, SHOC2, RANGAP1, XRRA1 |
| repeat: LRR 8 | 7 | NLRC5, SYNE2, PPP1R7, LRRC8B, LRRN3, LRRC8D, SHOC2 |
| repeat: LRR 5 | 13 | NLRC5, RSUl, SYNE2, PPP1R7, LRRC8B, LRRC41, LRRN3, LRRC8D, |
| | | LRRC59, LHCGR, SHOC2, RANGAP1, XRRA1 |
| SM00369: LRR_TYP | 9 | RSUl, PPP1R7, LRRC8B, CNOT6L, LRRN3, LRRC8D, LRRC59, SHOC2, |
| | | XRRA1 |
| IPR003591: Leucine-rich repeat, typical | 9 | RSUl, PPP1R7, LRRC8B, CNOT6L, LRRN3, LRRC8D, LRRC59, SHOC2, |
| subtype | | XRRA1 |
| repeat: LRR 4 | 14 | NLRC5, RSUl, SYNE2, PPP1R7, LRRC8B, LRRC41, LRRN3, LRRC8D, |
| | | LRRC59, LHCGR, FBXL5, SHOC2, RANGAP1, XRRA1 |
| repeat: LRR 3 | 16 | RSUl, LRRC8B, LRRC41, LRRC8D, LHCGR, LRRN3, SHOC2, RANGAP1, |
| | | XRRA1, NLRC5, SYNE2, PPP1R7, KDM2A, CNOT6L, LRRC59, FBXL5 |
| Leucine-rich repeat | 16 | RSUl, LRRC8B, LRRC41, LRRC8D, LHCGR, LRRN3, SHOC2, RANGAP1, |
| | | XRRA1, NLRC5, PPP1R7, KDM2A, CNOT6L, LRRC59,1413XL5, FBXL15 |
| repeat: LRR 1 | 17 | RSUl, LRRC8B, LRRC41, LRRC8D, LHCGR, LRRN3, SHOC2, RANGAP1, |
| | | XRRA1, NLRC5, SYNE2, KDM2A, PPP1R7, CNOT6L, LRRC59, FBXL5, |
| | | HECTD4 |
| repeat: LRR 2 | 17 | RSUl, LRRC8B, LRRC41, LRRC8D, LHCGR, LRRN3, SHOC2, RANGAP1, |
| | | XRRA1, NLRC5, SYNE2, KDM2A, PPP1R7, CNOT6L, LRRC59, FBXL5, |
| | | HECTD4 |
| IPR001611: Leucine- | 13 | RSUl, LRRC8B, LRRN3, LRRC8D, SHOC2, RANGAP1, XRRA1, NLRC5, |
| rich repeat | | PPP1R7, CNOT6L, LRRC59, FBXL5, FBXL15 |
| Enrichment Score: | | |
| 5.610490263279422E−4 | | |
| GO: 0006814~sodium ion transport | 4 | NDUFA9, COMMD3, SLC38A10, COMMD9 |
| Sodium | 5 | SLC20A2, COMMD3, SLC38A10, POLB, COMMD9 |
| Sodium transport | 4 | SLC20A2, COMMD3, SLC38A10, COMMD9 |
| Enrichment Score: | | |
| 2.1717103654966732E−4 | | |
| domain: Ig-like C2-type 4 | 3 | ICAM1, IGSF8, ICAM3 |
| domain: Ig-like C2-type 3 | 5 | ICAM1, IL18R1, IGSF8, ICAM3, LAG3 |
| domain: Ig-like C2-type 1 | 7 | ICAM1, IL18R1, IGSF8, ICAM3, MALT1, CD226, LAG3 |
| domain: Ig-like C2-type 2 | 7 | ICAM1, IL18R1, IGSF8, ICAM3, MALT1, CD226, LAG3 |
| Enrichment Score: | | |
| 1.8372944146090797E−4 | | |
| domain.C-type lectin | 3 | DGCR2, CD93, KLRD1 |
| SM00034: CLECT | 3 | DGCR2, CD93, KLRD1 |
| IPR016186: C-type lectin-like | 4 | DGCR2, CLECL1, CD93, KLRD1 |
| IPR001304: C-type lectin | 3 | DGCR2, CD93, KLRD1 |
| IPR016187: C-type lectin fold | 4 | DGCR2, CLECL1, CD93, KLRD1 |
| Lectin | 7 | GALNT2, BSG, DGCR2, CLECL1, CD93, KLRD1, LGALS9 |
| Enrichment Score: | | |
| 1.3260097487500437E−4 | | |
| repeat: 1 | 16 | BRF1, BRF2, VHL, TBP, GTF2B, IWS1, GLTP, PEF1, CCDC6, IGF2R, |
| | | KHSRP, HIVEP2, PCYT1A, PIAS1, RANBP2, NFATC2 |
| repeat: 2 | 15 | BRF1, BRF2, VHL, TBP, GTF2B, GLTP, IWS1, PEF1, CCDC6, IGF2R, |
| | | KHSRP, HIVEP2, RANBP2, PIAS1, NFATC2 |
| repeat: 8 | 4 | PEF1, VHL, IGF2R, HIVEP2 |
| repeat: 9 | 3 | PEF1, IGF2R, HIVEP2 |
| repeat: 7 | 4 | PEF1, VHL, IGF2R, HIVEP2 |
| repeat: 5 | 5 | PEF1, CCDC6, VHL, IGF2R, HIVEP2 |
| repeat: 6 | 4 | PEF1, VHL, IGF2R, HIVEP2 |
| repeat: 3 | 7 | PEF1, CCDC6, VHL, IGF2R, KHSRP, HIVEP2, PCYT1A |
| repeat: 4 | 5 | PEF1, VHL, IGF2R, KHSRP, HIVEP2 |
| Enrichment Score: | | |
| 3.460852670649958E−5 | | |
| Vision | 4 | UNC119, PDE6D, MKKS, CRYBB2 |
| GO: 0007601~visual | 8 | ABLIM1, ATF6, UNC119, DRAM2, PDE6D, MKKS, CLN6, CRYBB2 |
| perception | | |
| Sensory transduction | 5 | UNC119, PDE6D, MKKS, DTNBP1, CRYBB2 |
| Enrichment Score: | | |
| 8.483999996378984E−6 | | |
| Membrane | 712 | CCZ1B, CDIPT, USE1, VPS53, STRN, MPV17, RANGAP1, SLC52A2, IGHM, |
| | | FAM210A, TESPA1, ACBD5, TMEM140, TMEM147, ILK, VPS4A, JAGN1, |
| | | TMEM14B, GOLGA8A, KLRD1, IBTK, TIMMDC1, BSG, ROCK1, ROCK2, |
| | | UBE2J1, VPS41, UBE2J2, PIK3IP1, ERGIC1, BCL2L11, MARK2, TMEM131, |
| | | UNC13D, CD320, TMEM138, SIPA1L1, HLA-DPA1, ARL8B, CD226, ORAI1, |
| | | KIAA1109, MOB4, C16ORF91, RER1, CCDC91, ARF6, ARFGEF2, SLC29A1, |
| | | TMEM50B, SYPL1, RAC1, CKLF, ZAP70, HLA-DPB1, CDC42EP3, MGAT4A, |
| | | LAPTM4A, ATP11A, SPTSSA, MPC1, MPC2, APOL2, TMEM115, SYNE2, |
| | | APOL1, RGS1, ERVK13-1, ARF4, HGS, SMURF2, SYTL3, TMEM41A, |
| | | TMEM41B, C19ORF12, CD200, RTN4, LZTS3, ENOX2, SLC20A2, TSG101, |
| | | BNIP3, AP3S1, FAM169A, RTN3, OFD1, LNPEP, AP1S3, FAM168B, |
| | | TMEM109, SMAP1, SLMAP, IL4R, SPG21, CLDND1, MYB, LAG3, DPP4, |
| | | BRD8, PARVG, SYMPK, FMNL3, ZDHHC3, ARHGEF1, RAP2C, PIK3C2A, |
| | | AIFM1, ZDHHC8, KCTD20, GLCCI1, CLIC1, PI4KB, PJA2, BNIP1, BNIP2, |
| | | RIPK1, RRM1, TCHP, LRMP, OSBPL11, CHSY1, EIF5A2, ARL4C, |
| | | GRAMD1A, TMCO1, WDR45, ARL4A, CLN6, SNAP29, MFSD6, RABGAP1, |
| | | EXOC7, REPS1, CSF1, MFSD5, PML, ABHD3, EEA1, ABHD2, APBB1IP, |
| | | DGKE, DDX19A, NUP210, MPDU1, RAB11B, ILVBL, ARMCX3, CERK, |
| | | SNAP23, PCSK7, ACSL4, TRIP11, ACSL3, C1GALT1, ACSL5, SOAT1, |
| | | NBPF10, VTA1, CTLA4, DGKH, TMEM5, TTC17, TMEM2, NAE1, SDHA, |
| | | PLSCR1, MPHOSPH9, NRAS, P2RY10, PLEKHF2, CSNK1D, GSK3B, SDHC, |
| | | BNIP3L, CMTM7, DGKZ, TAPBPL, YKT6, CMTM3, NCLN, GNA13, AP1G1, |
| | | ATP6AP2, UNC50, HBS1L, TSPAN5, IDE, SLC7A6, ELOVL1, ST3GAL1, |
| | | MFF, PRKAR2A, SMIM7, PGRMC1, AAK1, SMIM8, PGRMC2, LRRC59, |
| | | RALB, DNAJC5, RNF149, SAR1B, DDOST, MATK, TMEM205, TMEM203, |
| | | SPTLC2, FMR1, STIM1, PSD4, ZNF7, PDE4D, TMEM208, HCST, PNPLA8, |
| | | TMEM106B, RNF139, VAMPS, PCMTD1, VAMP2, MFAP3, SLFN12L, |
| | | DCUN1D5, PHKA2, ARFGAP2, LITAF, IFITM1, TMEM214, IFITM2, STK10, |
| | | STAM2, LRBA, CERS6, TMEM219, SLC38A10, CDC42SE1, CERS4, TRDC, |
| | | P2RY8, DOCK2, FICD, PDE6D, CERS2, PCYT1A, RNF167, FKRP, SDF4, |
| | | TMEM30A, LYSMD3, TMEM223, HERPUD1, B4GALT3, PHACTR2, PRAF2, |
| | | TMEM222, MSMO1, SYT11, MYO1G, NIPA2, SNAPIN, PLGRKT, WIPI2, |
| | | ABCB7, FURIN, HERPUD2, NDUFV3, ATF6, TIGIT, LAMP1, LAMP2, TSC1, |
| | | NDUFV2, TRAF3IP3, PTTGlIP, NDUFAF4, LRRC8B, ATL3, LRRC8D, |
| | | CLSTN1, TMEM237, MFSD2A, PEX3, PIP5K1A, MBP, PEX2, SNTB1, VPS16, |
| | | AP5M1, CCDC107, PTDSS1, RHOF, NDUFS1, EBAG9, TBL1XR1, SIT1, |
| | | AVL9, C17ORF62, PKN2, STXBP2, TMEM248, BANP, NUP85, LPIN1, M6PR, |
| | | TMEM245, TIMM22, TMEM243, ARL3, SH2D3C, TNFSF13B, DEF6, TBCD, |
| | | IGF2R, GINM1, SLC35E1, SLC41A1, MFSD10, EMC1, SNX13, SNX11, |
| | | SNX19, GALNT2, TAPT1, NUP98, APH1A, TPRA1, PPM1A, NUP93, |
| | | TMEM259, ALDH3A2, APLP2, RNF125, NDC1, AP3M2, AP3M1, HRH2, |
| | | TSPAN31, SHISA5, SLC35B4, TMED1, SEC22B, PAFAH1B1, CCS, INPP5D, |
| | | EHD1, CD5, VP539, EHD4, GTF3C3, EBP, VHL, ATRAID, CBL, ANXA1, |
| | | RAF1, DPYSL2, BAD, TSPAN17, TMEM55B, ADI1, CLPTM1, LMBR1L, |
| | | IFIT5, JAK2, FAF1, GGA1, GGA3, VP525, RARRES3, SEC31B, TMEM19, |
| | | MRPL42, CHMP3, SEC31A, B3GALT6, ADCY7, IL6ST, VAPB, CHMP6, |
| | | NELL2, LHCGR, UTRN, CNPPD1, TMEM11, ZNRF1, PSKH1, ATP2B4, |
| | | VPS13C, GBF1, INSIG2, RAPGEF6, ERAP1, TMUB1, STAM, C60RF136, |
| | | SAYSD1, TBC1D9B, PLD3, DBNL, GPR137, CRLS1, CAPNS1, STRN3, |
| | | TRABD2A, RINT1, PIM1, NKTR, MGAT1, MGAT2, CD37, BTN3A1, RAB18, |
| | | ATP2C1, CCR4, LPAR6, ACAP1, ACAP2, CDCA7L, AMFR, TMEM184C, |
| | | ADD3, BTN3A2, ADD1, SUCO, TMX2, GPATCH2L, GLG1, CLCN3, LMNB2, |
| | | TMX3, BROX, NPIPB4, ASAP1, SFXN4, AKAP10, RFFL, PPAT, FAM65B, |
| | | SERINC3, SLC11A2, KRAS, SERINC1, STX17, EMB, STX10, GPR155, |
| | | ST6GAL1, OSBPL3, TM2D1, S100A10, SLAMF7, CD63, CDC27, BTLA, |
| | | CD55, ATP13A1, RNF4, CD59, KIAA0922, CNIH4, BET1L, RIT1, SPG7, |
| | | COX11, RAB5B, RAB5C, VPS37B, HELZ, CD151, HVCN1, ATG2B, FAR1, |
| | | EFHD2, ATAD3A, DNAJC15, GOLGA7, DNAJC16, PARL, GNPAT, |
| | | ATP6V0D1, ATP5H, C10ORF54, ICAM1, LPGAT1, JKAMP, ICAM3, VEZT, |
| | | CYB5A, FAM76B, RHBDD1, METTL2B, MIEN1, RHBDD2, TNFRSF10A, |
| | | GRM4, TRAP1, CHMP1A, ZDHHC16, NUS1, ZDHHC12, RAB5A, CNEP1R1, |
| | | ORMDL3, MOSPD1, FKBP11, MAP3K13, LIMS1, BET1, DNAJC30, SFT2D2, |
| | | LPXN, STT3A, PEX19, GNPTAB, TYW1, BCL2, PEX16, IPCEF1, CD27, |
| | | IL18R1, IMMT, LRRN3, BIRC6, ITPR2, SAMD8, COG3, COGS, DRAM2, |
| | | RAB30, SLC16A7, AP2A1, EEF1E1, MTFP1, RAB35, MBOAT1, RBM15, |
| | | FAM126B, GOLGB1, TSPO, CHMP4A, DSTYK, PI4K2B, STARD3, GLT8D1, |
| | | TIAM1, CPDX, MS4A1, SLC25A28, PRKACB, LEPROT, MCOLN2, MX1, |
| | | SPN, TOR1AIP2, SCAMP3, SMIM15, MADD, C2CD5, TOR1AIP1, CHP1, |
| | | FAM118A, MOGS, TIMM8A, BCAP31, SLC25A32, IGSF8, LRP10, USO1, |
| | | SLC25A38, SDCBP, KDSR, SMIM20, VPS26A, GBP3, DERL1, MCL1, ITGAE, |
| | | CEP95, TMEM63A, UNC93B1, SNX2, RABGAP1L, NAPA, SNX4, MUM1, |
| | | ITGB1, CASD1, PRR7, PEF1, FIS1, SLC30A5, ENTPD6, HECTD4, B3GNT2, |
| | | RUNX1, YIPF6, RYK, RAB33A, SREBF2, RAB33B, REEP5, PLEKHA3, |
| | | TXNDC11, RHOT1, CPNE1, RHOT2, CD79B, AHCYL1, SLC25A16, IFI6, |
| | | DCXR, PLEKHAl, COPA, OSBP, UQCRC1, CD8A, CD8B, UTY, ECHDC1, |
| | | ARHGAP17, CXCR3, CLTC, UQCRFS1, ARHGAP15, LSR, WHAMM, |
| | | PDCD1, DGCR2, CD93, PIGF, CXCR4, GOPC, NECAP2, NECAP1, ATP8B2, |
| | | MKKS, RANBP2, MSN, TM9SF4, IFNGR2, AKT2, TM9SF2, CCDC88B, |
| | | SYNRG, RALBP1, ELP6, PRKCI, MPP6, PIGS, TMEM189, LDLRAD4, |
| | | PRKCD, PRKCB, SACM1L, IFNAR1, IMMP1L, CD84, FAM134B, NUCB1, |
| | | TNFRSF9, FAM134C, IFNAR2, C5ORF15, CLECL1, KRIT1, TMEM69, CD81, |
| | | NUCB2, UBE2W, COMMD1, C16ORF54, SPAST, PRKD3, C7ORF73, GPR108, |
| | | FAM173B, FAM173A, NDUFB7, CD247, RSAD2, CYTH2, DTNBP1, |
| | | TNFRSF1A, PSTPIP1, TMEM87A, SLC39A6, APMAP, MR1, CCZ1, SLC39A3, |
| | | APC, STAMBP, IL2RB, ICA1, PTPRE, GIMAP5, PTPRA, SUN2, RGS19, |
| | | TMBIM1, NUP155, SLC10A3, RGS14, SIRT2, CYSTM1, GIMAP1, MPG, |
| | | TMEM43, SLC6A6, SLC25A53, C9ORF69, COMTD1 |
| Transmembrane | 455 | CDIPT, USE1, MPV17, SLC52A2, IGHM, FAM210A, ACBD5, TMEM140, |
| | | TMEM147, JAGN1, TMEM14B, KLRD1, TIMMDC1, BSG, UBE2J1, UBE2J2, |
| | | PIK3IP1, ERGIC1, TMEM131, CD320, TMEM138, HLA-DPA1, CD226, |
| | | ORAIl, KIAA1109, C16ORF91, RER1, SLC29A1, TMEM50B, SYPL1, CKLF, |
| | | HLA-DPB1, MGAT4A, LAPTM4A, ATP11A, MPC1, SPTSSA, MPC2, APOL2, |
| | | TMEM115, SYNE2, APOL1, ERVK13-1, ARF4, SMURF2, TMEM41A, |
| | | C19ORF12, TMEM41B, CD200, RTN4, SLC20A2, BNIP3, RTN3, FAM168B, |
| | | LNPEP, OFD1, TMEM109, IL4R, SLMAP, CLDND1, MYB, LAG3, DPP4, |
| | | BRD8, ZDHHC3, AIFM1, ZDHHC8, KCTD20, CLIC1, GLCCI1, BNIP1, |
| | | BNIP2, RRM1, LRMP, CHSY1, TMCO1, GRAMD1A, WDR45, CLN6, MFSD6, |
| | | RABGAP1, CSF1, MFSD5, ABHD3, ABHD2, DGKE, NUP210, MPDUL |
| | | ILVBL, ARMCX3, SNAP23, PCSK7, ACSL4, ACSL3, C1GALT1, ACSL5, |
| | | SOAT1, NBPF10, CTLA4, TMEM5, TMEM2, PLSCR1, P2RY10, SDHC, |
| | | BNIP3L, CMTM7, TAPBPL, CMTM3, NCLN, ATP6AP2, UNC50, TSPAN5, |
| | | HBS1L, SLC7A6, ST3GAL1, ELOVL1, MFF, SMIM7, PGRMC1, SMIM8, |
| | | LRRC59, PGRMC2, DNAJC5, RNF149, DDOST, TMEM205, TMEM203, |
| | | SPTLC2, STIM1, ZNF7, HCST, TMEM208, TMEM106B, PNPLA8, RNF139, |
| | | VAMPS, MFAP3, VAMP2, SLFN12L, DCUN1D5, TMEM214, IFITM1, LITAF, |
| | | IFITM2, LRBA, CERS6, TMEM219, SLC38A10, CERS4, TRDC, P2RY8, FICD, |
| | | CERS2, RNF167, FKRP, TMEM30A, LYSMD3, TMEM223, HERPUD1, |
| | | B4GALT3, PRAF2, MSMO1, TMEM222, SYT11, NIPA2, PLGRKT, ABCB7, |
| | | HERPUD2, FURIN, TIGIT, ATF6, LAMP1, LAMP2, TRAF3IP3, PTTGHP, |
| | | LRRC8B, ATL3, LRRC8D, CLSTN1, TMEM237, MFSD2A, PEX3, PEX2, |
| | | PTDSS1, CCDC107, EBAG9, TBL1XR1, SIT1, AVL9, C17ORF62, PKN2, |
| | | STXBP2, TMEM248, BANP, TMEM245, M6PR, TMEM243, TIMM22, |
| | | TNFSF13B, IGF2R, GINM1, SLC35E1, SLC41A1, MFSD10, EMC1, SNX13, |
| | | TAPT1, GALNT2, APH1A, TPRA1, PPM1A, TMEM259, APLP2, ALDH3A2, |
| | | NDC1, HRH2, TSPAN31, SLC35B4, SHISA5, TMED1, SEC22B, CCS, CD5, |
| | | GTF3C3, EBP, ATRAID, TSPAN17, TMEM55B, CLPTM1, LMBR1L, FAF1, |
| | | RARRES3, MRPL42, TMEM19, ADCY7, B3GALT6, IL6ST, VAPB, NELL2, |
| | | LHCGR, CNPPD1, TMEM11, ATP2B4, INSIG2, TMUB1, ERAP1, C6ORF136, |
| | | SAYSD1, TBC1D9B, PLD3, GPR137, CRLS1, TRABD2A, MGAT1, MGAT2, |
| | | CD37, BTN3A1, ATP2C1, LPAR6, CCR4, CDCA7L, AMFR, TMEM184C, |
| | | BTN3A2, SUCO, GPATCH2L, GLG1, TMX2, CLCN3, TMX3, NPIPB4, |
| | | ASAP1, SFXN4, PPAT, SERINC3, SLC11A2, STX17, SERINC1, EMB, STX10, |
| | | GPR155, ST6GAL1, TM2D1, S100A10, SLAMF7, CD63, CDC27, BTLA, |
| | | ATP13A1, RNF4, KIAA0922, CNIH4, BET1L, SPG7, COX11, HELZ, CD151, |
| | | HVCN1, FAR1, DNAJC15, ATAD3A, DNAJC16, PARL, C10ORF54, ICAM1, |
| | | JKAMP, LPGAT1, ICAM3, CYB5A, VEZT, FAM76B, METTL2B, RHBDD1, |
| | | RHBDD2, TNFRSF10A, GRM4, ZDHHC16, NUS1, ZDHHC12, CNEP1R1, |
| | | ORMDL3, MOSPD1, FKBP11, BET1, DNAJC30, SFT2D2, STT3A, GNPTAB, |
| | | BCL2, TYW1, PEX16, CD27, IL18R1, IMMT, LRRN3, ITPR2, SAMD8, |
| | | DRAM2, SLC16A7, MTFP1, EEF1E1, MBOAT1, GOLGB1, TSPO, STARD3, |
| | | GLT8D1, CPDX, MS4A1, SLC25A28, MCOLN2, LEPROT, SPN, TOR1AIP2, |
| | | SCAMP3, SMIM15, TOR1AIP1, FAM118A, MOGS, BCAP31, SLC25A32, |
| | | IGSF8, LRP10, SLC25A38, SDCBP, KDSR, SMIM20, DERL1, MCL1, CEP95, |
| | | ITGAE, TMEM63A, UNC93B1, RABGAP1L, MUM1, ITGB1, CASD1, PRR7, |
| | | FIS1, SLC30A5, ENTPD6, HECTD4, B3GNT2, RUNX1, YIPF6, RYK, SREBF2, |
| | | REEP5, TXNDC11, RHOT1, RHOT2, CD79B, SLC25A16, IF16, CD8A, CD8B, |
| | | UTY, ECHDC1, CXCR3, UQCRFS1, LSR, PDCD1, DGCR2, CD93, PIGF, |
| | | CXCR4, MKKS, ATP8B2, TM9SF4, IFNGR2, TM9SF2, ELP6, PIGS, |
| | | TMEM189, LDLRAD4, IFNAR1, SACM1L, FAM134B, CD84, IFNAR2, |
| | | FAM134C, C5ORF15, TNFRSF9, CLECL1, TMEM69, CD81, UBE2W, |
| | | C16ORF54, SPAST, C7ORF73, GPR108, FAM173B, FAM173A, CD247, |
| | | TNFRSF1A, TMEM87A, SLC39A6, MR1, APMAP, SLC39A3, IL2RB, ICA1, |
| | | PTPRE, GIMAP5, PTPRA, SUN2, TMBIM1, SLC10A3, CYSTM1, GIMAP1, |
| | | MPG, TMEM43, SLC6A6, C9ORF69, SLC25A53, COMTD1 |
| Transmembrane | 453 | CDIPT, USE1, MPV17, SLC52A2, FAM210A, ACBD5, TMEM140, TMEM147, |
| helix | | JAGN1, TMEM14B, KLRD1, TIMMDC1, BSG, UBE2J1, UBE2J2, PIK3IP1, |
| | | ERGIC1, TMEM131, CD320, TMEM138, HLA-DPA1, CD226, ORAIl, |
| | | KIAA1109, C16ORF91, RER1, SLC29A1, TMEM50B, SYPL1, CKLF, HLA- |
| | | DPB1, MGAT4A, LAPTM4A, ATP11A, MPC1, SPTSSA, MPC2, APOL2, |
| | | TMEM115, SYNE2, APOL1, ERVK13-1, ARF4, SMURF2, TMEM41A, |
| | | C19ORF12, TMEM41B, CD200, RTN4, SLC20A2, BNIP3, RTN3, FAM168B, |
| | | LNPEP, OFD1, TMEM109, IL4R, SLMAP, CLDND1, MYB, LAG3, DPP4, |
| | | BRD8, ZDHHC3, AIFM1, ZDHHC8, KCTD20, CLIC1, GLCCI1, BNIP1, |
| | | BNIP2, RRM1, LRMP, CHSY1, TMCO1, GRAMD1A, WDR45, CLN6, MFSD6, |
| | | RABGAP1, CSF1, MFSD5, ABHD3, ABHD2, DGKE, NUP210, MPDUL |
| | | ILVBL, ARMCX3, SNAP23, PCSK7, ACSL4, ACSL3, C1GALT1, ACSL5, |
| | | SOAT1, NBPF10, CTLA4, TMEM5, TMEM2, PLSCR1, P2RY10, SDHC, |
| | | BNIP3L, CMTM7, TAPBPL, CMTM3, NCLN, ATP6AP2, UNC50, TSPAN5, |
| | | HBS1L, SLC7A6, ST3GAL1, ELOVL1, MFF, SMIM7, PGRMC1, SMIM8, |
| | | LRRC59, PGRMC2, DNAJC5, RNF149, DDOST, TMEM205, TMEM203, |
| | | SPTLC2, STIM1, ZNF7, HCST, TMEM208, TMEM106B, PNPLA8, RNF139, |
| | | VAMPS, MFAP3, VAMP2, SLFN12L, DCUN1D5, TMEM214, IFITM1, LITAF, |
| | | IFITM2, LRBA, CERS6, TMEM219, SLC38A10, CERS4, TRDC, P2RY8, FICD, |
| | | CERS2, RNF167, FKRP, TMEM30A, LYSMD3, TMEM223, HERPUD1, |
| | | B4GALT3, PRAF2, MSM01, TMEM222, SYT11, NIPA2, PLGRKT, ABCB7, |
| | | HERPUD2, FURIN, TIGIT, ATF6, LAMP1, LAMP2, TRAF3IP3, PTTGHP, |
| | | LRRC8B, ATL3, LRRC8D, CLSTN1, TMEM237, MFSD2A, PEX3, PEX2, |
| | | PTDSS1, CCDC107, EBAG9, TBL1XR1, SIT1, AVL9, C17ORF62, PKN2, |
| | | STXBP2, TMEM248, BANP, TMEM245, M6PR, TMEM243, TIMM22, |
| | | TNFSF13B, IGF2R, GINM1, SLC35E1, SLC41A1, MFSD10, EMC1, SNX13, |
| | | TAPT1, GALNT2, APH1A, TPRA1, PPM1A, TMEM259, APLP2, ALDH3A2, |
| | | NDC1, HRH2, TSPAN31, SLC35B4, SHISA5, TMED1, SEC22B, CCS, CD5, |
| | | GTF3C3, EBP, ATRAID, TSPAN17, TMEM55B, CLPTM1, LMBR1L, FAF1, |
| | | RARRES3, MRPL42, TMEM19, ADCY7, B3GALT6, IL6ST, VAPB, NELL2, |
| | | LHCGR, CNPPD1, TMEM11, ATP2B4, INSIG2, TMUB1, ERAP1, C6ORF136, |
| | | SAYSD1, TBC1D9B, PLD3, GPR137, CRLS1, TRABD2A, MGAT1, MGAT2, |
| | | CD37, BTN3A1, ATP2C1, LPAR6, CCR4, CDCA7L, AMFR, TMEM184C, |
| | | BTN3A2, SUCO, GPATCH2L, GLG1, TMX2, CLCN3, TMX3, NPIPB4, |
| | | ASAP1, SFXN4, PPAT, SERINC3, SLC11A2, STX17, SERINCL EMB, STX10, |
| | | GPR155, ST6GAL1, TM2D1, S100A10, SLAMF7, CD63, CDC27, BTLA, |
| | | ATP13A1, RNF4, KIAA0922, CNIH4, BET1L, SPG7, COX11, HELZ, CD151, |
| | | HVCN1, FAR1, DNAJC15, ATAD3A, DNAJC16, PARL, C10ORF54, ICAM1, |
| | | JKAMP, LPGAT1, ICAM3, CYB5A, VEZT, FAM76B, METTL2B, RHBDD1, |
| | | RHBDD2, TNFRSF10A, GRM4, ZDHHC16, NUS1, ZDHHC12, CNEP1R1, |
| | | ORMDL3, MOSPD1, FKBP11, BET1, DNAJC30, SFT2D2, STT3A, GNPTAB, |
| | | BCL2, TYW1, PEX16, CD27, IL18R1, IMMT, LRRN3, ITPR2, SAMD8, |
| | | DRAM2, SLC16A7, MTFP1, EEF1E1, MBOAT1, GOLGB1, TSPO, STARD3, |
| | | GLT8D1, CPDX, MS4A1, SLC25A28, MCOLN2, LEPROT, SPN, TOR1AIP2, |
| | | SCAMP3, SMIM15, TOR1AIP1, FAM118A, MOGS, BCAP31, SLC25A32, |
| | | IGSF8, LRP10, SLC25A38, SDCBP, KDSR, SMIM20, DERL1, MCL1, CEP95, |
| | | ITGAE, TMEM63A, UNC93B1, RABGAP1L, MUM1, ITGB1, CASD1, PRR7, |
| | | FIS1, SLC30A5, ENTPD6, HECTD4, B3GNT2, YIPF6, RYK, SREBF2, REEP5, |
| | | TXNDC11, RHOT1, RHOT2, CD79B, SLC25A16, IF16, CD8A, CD8B, UTY, |
| | | ECHDC1, CXCR3, UQCRFS1, LSR, PDCD1, DGCR2, CD93, PIGF, CXCR4, |
| | | MKKS, ATP8B2, TM9SF4, IFNGR2, TM9SF2, ELP6, PIGS, TMEM189, |
| | | LDLRAD4, IFNAR1, SACM1L, FAM134B, CD84, IFNAR2, FAM134C, |
| | | C5ORF15, TNFRSF9, CLECL1, TMEM69, CD81, UBE2W, C160RF54, SPAST, |
| | | C7ORF73, GPR108, FAM173B, FAM173A, CD247, TNFRSF1A, TMEM87A, |
| | | SLC39A6, MR1, APMAP, SLC39A3, IL2RB, ICA1, PTPRE, GIMAP5, PTPRA, |
| | | SUN2, TMBIM1, SLC10A3, CYSTM1, GIMAP1, MPG, TMEM43, SLC6A6, |
| | | C9ORF69, SLC25A53, COMTD1 |
| GO: 0016021~integral | 431 | CDIPT, USE1, MPV17, VPS51, IGHM, FAM210A, ACBD5, TMEM140, |
| component of | | TMEM147, JAGN1, TMEM14B, KLRD1, TIMMDC1, BSG, UBE2J1, UBE2J2, |
| membrane | | PIK3IP1, ERGIC1, TMEM131, CD320, TMEM138, HLA-DPA1, CD226, |
| | | ORAIl, KIAA1109, C16ORF91, RER1, TMEM50B, SYPL1, CKLF, HLA- |
| | | DPB1, MGAT4A, LAPTM4A, ATP11A, MPC1, SPTSSA, MPC2, APOL2, |
| | | TMEM115, SYNE2, APOL1, ERVK13-1, ARF4, SMURF2, TMEM41A, |
| | | C19ORF12, TMEM41B, CD200, RTN4, SLC20A2, BNIP3, RTN3, FAM168B, |
| | | OFD1, TMEM109, IL4R, SLMAP, CLDND1, MYB, LAG3, DPP4, BRD8, |
| | | ZDHHC3, AIFM1, ZDHHC8, KCTD20, GLCCI1, BNIP2, RRM1, LRMP, |
| | | CHSY1, TMCO1, GRAMD1A, WDR45, CLN6, MFSD6, RABGAP1, MFSD5, |
| | | CSF1, ABHD3, ABHD2, DGKE, NUP210, MPDU1, ILVBL, ARMCX3, PCSK7, |
| | | SNAP23, CERK, ACSL4, ACSL3, C1GALT1, ACSL5, SOAT1, NBPF10, |
| | | CTLA4, TMEM5, TMEM2, P2RY10, SDHC, BNIP3L, CMTM7, TAPBPL, |
| | | CMTM3, YKT6, NCLN, ATP6AP2, UNC50, TSPAN5, HBS1L, SLC7A6, |
| | | ST3GAL1, ELOVL1, MFF, SMIM7, PGRMC1, LRRC59, PGRMC2, SMIM8, |
| | | DNAJC5, RNF149, DDOST, TMEM205, TMEM203, SPTLC2, ZNF7, HCST, |
| | | TMEM208, TMEM106B, PNPLA8, RNF139, MFAP3, VAMP2, SLFN12L, |
| | | DCUN1D5, TMEM214, IFITM1, LITAF, IFITM2, LRBA, CERS6, TMEM219, |
| | | SLC38A10, CERS4, TRDC, P2RY8, FICD, CERS2, RNF167, FKRP, |
| | | TMEM30A, LYSMD3, TMEM223, HERPUD1, B4GALT3, PRAF2, MSMO1, |
| | | TMEM222, NIPA2, ABCB7, HERPUD2, FURIN, TIGIT, LAMP1, LAMP2, |
| | | TRAF3IP3, PTTGlIP, LRRC8B, ATL3, LRRC8D, CLSTN1, TMEM237, |
| | | MFSD2A, PEX2, PTDSS1, CCDC107, EBAG9, TBL1XR1, SIT1, AVL9, |
| | | C17ORF62, PKN2, STXBP2, TMEM248, BANP, TMEM245, M6PR, |
| | | TMEM243, TIMM22, TNFSF13B, IGF2R, GINM1, SLC35E1, MFSD10, |
| | | SLC41A1, EMC1, SNX13, TAPT1, GALNT2, APH1A, TPRA1, PPM1A, |
| | | TMEM259, APLP2, ALDH3A2, NDC1, TSPAN31, SLC35B4, SHISA5, TMED1, |
| | | SEC22B, CCS, CD5, GTF3C3, EBP, ATRAID, TSPAN17, TMED8, TMEM55B, |
| | | CLPTM1, LMBR1L, FAF1, RARRES3, MRPL42, TMEM19, ADCY7, |
| | | B3GALT6, IL6ST, VAPB, NELL2, LHCGR, CNPPD1, ATP2B4, INSIG2, |
| | | TMUB1, ERAP1, C6ORF136, SAYSD1, TBC1D9B, PLD3, GPR137, CRLS1, |
| | | MGAT1, MGAT2, BTN3A1, CD37, ATP2C1, LPAR6, CCR4, CDCA7L, AMFR, |
| | | TMEM184C, BTN3A2, SUCO, GPATCH2L, GLG1, TMX2, CLCN3, TMX3, |
| | | NPIPB4, SFXN4, ASAP1, PPAT, SERINC3, SLC11A2, STX17, SERINC1, |
| | | EMB, STX10, GPR155, ST6GAL1, TM2D1, S100A10, SLAMF7, CD63, |
| | | CDC27, BTLA, ATP13A1, RNF4, KIAA0922, CNIH4, BET1L, COX11, SPG7, |
| | | HELZ, CD151, HVCN1, FAR1, DNAJC15, ATAD3A, DNAJC16, PARL, |
| | | C10ORF54, ICAM1, JKAMP, LPGAT1, ICAM3, CYB5A, VEZT, FAM76B, |
| | | METTL2B, RHBDD1, RHBDD2, TNFRSF10A, GRM4, ZDHHC16, NUS1, |
| | | ZDHHC12, CNEP1R1, ORMDL3, MOSPD1, FKBP11, BET1, SEC14L1, |
| | | DNAJC30, SFT2D2, STT3A, GNPTAB, PEX19, BCL2, TYVV1, IL18R1, IMMT, |
| | | LRRN3, ITPR2, SAMD8, DRAM2, MTFP1, EEF1E1, MBOAT1, GOLGB1, |
| | | TSPO, STARD3, GLT8D1, CPDX, SLC25A28, MCOLN2, LEPROT, SPN, |
| | | TOR1AIP2, SCAMP3, SMIM15, MADD, TOR1AIP1, FAM118A, MOGS, |
| | | BCAP31, SLC25A32, IGSF8, LRP10, SLC25A38, SDCBP, KDSR, SMIM20, |
| | | DERL1, MCL1, CEP95, TMEM63A, UNC93B1, RABGAP1L, MUM1, CASD1, |
| | | PRR7, SLC30A5, ENTPD6, HECTD4, B3GNT2, RUNX1, YIPF6, RYK, REEP5, |
| | | TXNDC11, RHOT1, SLC25A16, IFI6, CD8A, CD8B, UTY, ECHDC1, CXCR3, |
| | | LSR, PDCD1, DGCR2, PIGF, CD93, CXCR4, MKKS, ATP8B2, TM9SF4, |
| | | IFNGR2, TM9SF2, ELP6, TMEM189, LDLRAD4, SACM1L, IFNAR2, |
| | | FAM134C, C5ORF15, TNFRSF9, CLECL1, SBF1, TMEM69, CD81, UBE2W, |
| | | C16ORF54, SPAST, C7ORF73, GPR108, FAM173B, FAM173A, CD247, |
| | | TNFRSF1A, TMEM87A, SLC39A6, MR1, APMAP, SLC39A3, ICA1, PTPRE, |
| | | GIMAP5, PTPRA, TMBIM1, SLC10A3, CYSTM1, GIMAP1, MPG, TMEM43, |
| | | SLC6A6, C9ORF69, SLC25A53, COMTD1 |
| transmembrane | 387 | CDIPT, USE1, MPV17, SLC52A2, FAM210A, ACBD5, TMEM140, TMEM147, |
| region | | JAGN1, TMEM14B, KLRD1, TIMMDC1, BSG, UBE2J1, UBE2J2, PIK3IP1, |
| | | ERGIC1, TMEM131, CD320, TMEM138, HLA-DPA1, CD226, ORAIl, |
| | | KIAA1109, C16ORF91, RER1, TMEM50B, SLC29A1, SYPL1, CKLF, HLA- |
| | | DPB1, MGAT4A, LAPTM4A, SPTSSA, ATP11A, TMEM115, SYNE2, |
| | | TMEM41A, C19ORF12, TMEM41B, CD200, RTN4, SLC20A2, BNIP3, RTN3, |
| | | LNPEP, TMEM109, IL4R, SLMAP, CLDND1, DPP4, LAG3, ZDHHC3, |
| | | ZDHHC8, CLIC1, BNIP1, LRMP, CHSY1, TMCO1, GRAMD1A, CLN6, |
| | | MFSD6, MFSD5, CSF1, ABHD3, ABHD2, DGKE, NUP210, MPDU1, |
| | | ARMCX3, ILVBL, PCSK7, ACSL4, ACSL3, C1GALT1, ACSL5, SOAT1, |
| | | CTLA4, TMEM5, TMEM2, PLSCR1, P2RY10, SDHC, BNIP3L, CMTM7, |
| | | TAPBPL, CMTM3, NCLN, ATP6AP2, TSPAN5, UNC50, SLC7A6, ST3GAL1, |
| | | ELOVL1, MFF, SMIM7, PGRMC1, LRRC59, PGRMC2, SMIM8, RNF149, |
| | | DDOST, TMEM205, TMEM203, SPTLC2, STIM1, HCST, TMEM208, |
| | | TMEM106B, PNPLA8, VAMPS, RNF139, MFAP3, VAMP2, TMEM214, |
| | | IFITM1, IFITM2, CERS6, LRBA, SLC38A10, TMEM219, CERS4, P2RY8, |
| | | FICD, CERS2, RNF167, TMEM30A, LYSMD3, TMEM223, HERPUD1, PRAF2, |
| | | B4GALT3, MSMO1, TMEM222, SYT11, NIPA2, PLGRKT, ABCB7, |
| | | HERPUD2, FURIN, TIGIT, ATF6, LAMP1, LAMP2, TRAF3IP3, PTTGlIP, |
| | | LRRC8B, CLSTN1, LRRC8D, ATL3, TMEM237, MFSD2A, PEX3, PEX2, |
| | | PTDSS1, CCDC107, EBAG9, SIT1, AVL9, C17ORF62, TMEM248, TMEM245, |
| | | M6PR, TMEM243, TIMM22, TNFSF13B, GINM1, IGF2R, SLC35E1, MFSD10, |
| | | SLC41A1, EMC1, TAPT1, GALNT2, APH1A, TPRA1, TMEM259, APLP2, |
| | | ALDH3A2, NDC1, TSC22D3, HRH2, TSPAN31, TMED1, SLC35B4, SHISA5, |
| | | SEC22B, CD5, EBP, ATRAID, TSPAN17, TMEM55B, CLPTM1, LMBR1L, |
| | | ALKBH5, TMEM19, CHMP3, ADCY7, B3GALT6, VAPB, IL6ST, LHCGR, |
| | | CNPPD1, TMEM11, ATP2B4, INSIG2, TMUB1, ERAP1, SAYSD1, TBC1D9B, |
| | | PLD3, GPR137, CRLS1, TRABD2A, MGAT1, MGAT2, BTN3A1, CD37, |
| | | LPAR6, CCR4, ATP2C1, TMEM184C, AMFR, BTN3A2, SUCO, GPATCH2L, |
| | | GLG1, TMX2, CLCN3, TMX3, SFXN4, SERINC3, SLC11A2, STX17, |
| | | SERINC1, EMB, STX10, GPR155, ST6GAL1, TM2D1, SLAMF7, CD63, BTLA, |
| | | ATP13A1, CNIH4, KIAA0922, BET1L, COX11, SPG7, HVCN1, CD151, FAR1, |
| | | DNAJC15, DNAJC16, PARL, ICAM1, C10ORF54, JKAMP, LPGAT1, ICAM3, |
| | | CYB5A, VEZT, RHBDD1, RHBDD2, TNFRSF10A, GRM4, ZDHHC16, NUS1, |
| | | ZDHHC12, CNEP1R1, MOSPD1, ORMDL3, FKBP11, BET1, SFT2D2, STT3A, |
| | | GNPTAB, BCL2, PEX16, CD27, IL18R1, EPB41L4A-AS1, IMMT, LRRN3, |
| | | ITPR2, SAMD8, DRAM2, MBOAT1, GOLGB1, TSPO, STARD3, GLT8D1, |
| | | MS4A1, SLC25A28, MCOLN2, LEPROT, SPN, TOR1AIP2, SCAMP3, |
| | | SMIM15, MADD, TOR1AIP1, FAM118A, MOGS, BCAP31, SLC25A32, IGSF8, |
| | | LRP10, SLC25A38, KDSR, SMIM20, GBP3, DERL1, MCL1, ITGAE, |
| | | UNC93B1, TMEM63A, ITGB1, CASD1, PRR7, FIS1, ENTPD6, SLC30A5, |
| | | HECTD4, B3GNT2, YIPF6, RYK, REEP5, SREBF2, TXNDC11, RHOT1, |
| | | RHOT2, CD79B, SLC25A16, IFI6, CD8A, CD8B, CXCR3, LSR, PDCD1, |
| | | DGCR2, PIGF, CD93, CXCR4, ATP8B2, TM9SF4, IFNGR2, TM9SF2, PIGS, |
| | | TMEM189, LDLRAD4, IFNAR1, SACM1L, FAM134B, CD84, IFNAR2, |
| | | FAM134C, C5ORF15, TNFRSF9, CLECL1, TMEM69, CD81, C160RF54, |
| | | SPAST, GPR108, FAM173B, FAM173A, CD247, TNFRSF1A, SLC39A6, |
| | | TMEM87A, MR1, APMAP, SLC39A3, IL2RB, GIMAP5, PTPRE, PTPRA, |
| | | SUN2, TMBIM1, SLC10A3, GIMAP1, RPAP2, TMEM43, SLC6A6, SLC25A53, |
| | | COMTD1 |
| topological | 213 | B3GALT6, ADCY7, VAPB, IL6ST, LHCGR, USE1, TMEM140, ATP2B4, |
| domain: Cytoplasmic | | ERAP1, JAGN1, KLRD1, PLD3, GPR137, BSG, TRABD2A, UBE2J1, UBE2J2, |
| | | PIK3IP1, ERGIC1, MGAT1, MGAT2, CD37, BTN3A1, CD320, ATP2C1, CCR4, |
| | | LPAR6, HLA-DPA1, AMFR, CD226, BTN3A2, GLG1, TMX2, ORAIl, CLCN3, |
| | | C16ORF91, TMX3, SLC11A2, SERINC3, SLC29A1, SYPL1, STX17, SERINC1, |
| | | HLA-DPB1, EMB, STX10, MGAT4A, ST6GAL1, SPTSSA, ATP11A, SLAMF7, |
| | | CD63, BTLA, ATP13A1, SYNE2, KIAA0922, BET1L, CD200, RTN4, |
| | | SLC20A2, CD151, HVCN1, LNPEP, DNAJC16, IL4R, SLMAP, LAG3, DPP4, |
| | | ICAM1, C10ORF54, ZDHHC3, JKAMP, ZDHHC8, ICAM3, TNFRSF10A, |
| | | GRM4, ZDHHC16, BNIP1, NUS1, LRMP, CHSY1, CSF1, BET1, SFT2D2, |
| | | STT3A, NUP210, PEX16, PCSK7, ACSL4, ACSL3, C1GALT1, CD27, ACSL5, |
| | | IL18R1, EPB41L4A-AS1, LRRN3, CTLA4, TMEM5, ITPR2, SAMD8, PLSCR1, |
| | | P2RY10, TAPBPL, NCLN, GOLGB1, ATP6AP2, UNC50, TSPAN5, SLC7A6, |
| | | ST3GAL1, GLT8D1, STARD3, MFF, SMIM7, LRRC59, MS4A1, DDOST, SPN, |
| | | SCAMP3, STIM1, MOGS, HCST, BCAP31, IGSF8, LRP10, VAMPS, KDSR, |
| | | VAMP2, MFAP3, DERL1, ITGAE, CERS6, ITGB1, PRR7, P2RY8, FIS1, |
| | | CERS2, ENTPD6, SLC30A5, B3GNT2, LYSMD3, PRAF2, B4GALT3, |
| | | TMEM222, RYK, SYT11, NIPA2, SREBF2, ATF6, TIGIT, LAMP1, LAMP2, |
| | | TRAF3IP3, PTTGlIP, RHOT1, RHOT2, CD79B, CD8A, CD8B, ATL3, |
| | | CLSTN1, PEX3, CXCR3, LSR, PDCD1, DGCR2, CD93, CXCR4, ATP8B2, |
| | | IFNGR2, EBAG9, TM9SF2, SIT1, PIGS, M6PR, LDLRAD4, IFNAR1, CD84, |
| | | TNFRSF9, C5ORF15, IFNAR2, TNFSF13B, CLECL1, GINM1, IGF2R, CD81, |
| | | EMC1, GALNT2, CD247, ALDH3A2, APLP2, NDC1, TNFRSF1A, HRH2, |
| | | TSPAN31, TMED1, SHISA5, SEC22B, SLC39A6, MR1, APMAP, SLC39A3, |
| | | CD5, IL2RB, PTPRE, GIMAP5, ATRAID, PTPRA, TSPAN17, GIMAP1, |
| | | CLPTM1, LMBR1L, SLC6A6 |
| Enrichment Score: | | |
| 6.831574760707297E−6 | | |
| IPR003961: Fibronectin, type III | 9 | ATF7IP, IFNAR2, IL2RB, IL6ST, IL4R, LRRN3, IFNGR2, ATF7IP2, IFNAR1 |
| domain: Fibronectin type-III 2 | 3 | IL6ST, IFNGR2, IFNAR1 |
| domain: Fibronectin type-III 1 | 3 | IL6ST, IFNGR2, IFNAR1 |
| Enrichment Score: | | |
| 1.9767078533785107E−10 | | |
| IPR013032: EGF-like, conserved site | 3 | CD93, ATRAID, NELL2 |
| EGF-like domain | 3 | CD93, ATRAID, NELL2 |
| IPR000742: Epidermal | 3 | CD93, ATRAID, NELL2 |
| growth factor-like domain | | |
| Enrichment Score: | | |
| 0.0 | | |
| disulfide bond | 121 | IL6ST, LHCGR, NELL2, GEER, IGHM, ST3GAL1, MS4A1, KLRD1, BSG, |
| | | PMCH, PIK3IP1, TIMM8A, IGSF8, BTN3A1, NPC2, CD320, LRP10, LPAR6, |
| | | CCR4, HLA-DPA1, MFAP3, CD226, BTN3A2, CCL3, TXN2, ITGAE, TMX3, |
| | | CTSA, CCL4, ITGB1, LIF, BLOC1S5, ENTPD6, PCYT1A, HLA-DPB1, EMB, |
| | | ST6GAL1, B4GALT3, S100A11, MALT1, SLAMF7, PMF1, FURIN, TIGIT, |
| | | DNASE2, BTLA, TXNDC12, LAMP1, CD55, TXNDC11, LAMP2, CD59, |
| | | CD79B, XCL1, CD200, XCL2, CD8A, FAM3C, CD8B, HEXB, PDIA4, CXCR3, |
| | | UQCRFS1, LSR, PDCD1, DGCR2, CD93, CXCR4, IL4R, LAG3, DPP4, ICAM1, |
| | | C10ORF54, SIT1, AIFM1, LY96, ICAM3, GZMB, CLIC1, LDLRAD4, MIEN1, |
| | | IFNAR1, CD84, TNFRSF10A, TNFRSF9, IFNAR2, CTSL, TNFSF13B, IGF2R, |
| | | CD81, RRM1, MGEA5, TXNRD1, CTSC, CSF2, GALNT2, SPOCK2, CSF1, |
| | | CD247, APLP2, GLRX2, TNFRSF1A, GNPTAB, HRH2, PITRM1, CCS, MR1, |
| | | CD5, CD27, GLRX, IL18R1, CES2, IL2RB, ATRAID, LRRN3, CTLA4, |
| | | P2RY10, GLA, IRF3, TAPBPL, IL2 |
| Disulfide bond | 145 | IL6ST, LHCGR, NELL2, PTPN22, GFER, IGHM, SLC7A6, ST3GAL1, MS4A1, |
| | | ERAP1, KLRD1, BSG, PMCH, PIK3IP1, TIMM8A, HCST, MGAT1, IGSF8, |
| | | BTN3A1, UHRF2, NPC2, CD320, LRP10, LPAR6, CCR4, HLA-DPA1, MFAP3, |
| | | CD226, BTN3A2, MPST, CCL3, TXN2, ITGAE, TMX3, CTSA, CHCHD4, |
| | | TRDC, HEXDC, CCL4, ITGB1, LIF, RAC1, ENTPD6, HLA-DPB1, EMB, |
| | | FKRP, ST6GAL1, B4GALT3, S100A11, MALT1, SLAMF7, FURIN, BTLA, |
| | | TIGIT, DNASE2, TXNDC12, LAMP1, LAMP2, CD55, TXNDC11, ERVK13-1, |
| | | CD59, CD79B, XCL1, CD200, XCL2, BACH2, CD8A, FAM3C, CD8B, HEXB, |
| | | CXCR3, PDIA4, UQCRFS1, LSR, PDCD1, DGCR2, CD93, TPP1, CXCR4, |
| | | IL4R, RANBP2, LAG3, DPP4, AKT2, ICAM1, C10ORF54, SIT1, LY96, |
| | | ICAM3, GZMB, CLIC1, LDLRAD4, MIEN1, IFNAR1, CD84, TNFRSF10A, |
| | | TNFRSF9, GRM4, CTSL, IFNAR2, TNFSF13B, IGF2R, CD81, RRM1, |
| | | TXNRD1, CTSC, CSF2, GALNT2, NDUFB7, SPOCK2, CSF1, USPS, CD247, |
| | | APLP2, GLRX2, MYCBP2, TNFRSF1A, GNPTAB, HRH2, PITRM1, CCS, |
| | | MR1, CD5, C1GALT1, CD27, GLRX, SOAT1, IL18R1, CES2, IL2RB, |
| | | ATRAID, LRRN3, ANXA1, CTLA4, RAF1, NUP155, P2RY10, GLA, IRF3, |
| | | TAPBPL, POFUT1,I,EZ2, GOLGB1, IL2 |
| signal peptide | 165 | ATP6AP2, IL6ST, NELL2, LHCGR, SLC52A2, IGHM, SMIM7, IFNG, RNF149, |
| | | DDOST, IZUM04, SPN, GPR137, BSG, PMCH, TRABD2A, STIM1, PIK3IP1, |
| | | CECR5, HCST, IGSF8, ABHD17B, BTN3A1, CHID1, NPC2, LRP10, CD320, |
| | | HLA-DPA1, KDSR, MFAP3, AMFR, CD226, UGGT1, BTN3A2, SUCO, |
| | | HSD17B11, GLG1, TMX2, CCL3, C16ORF91, ITGAE, TMX3, CTSA, CCL4, |
| | | ITGB1, CASD1, LIF, BLOC1S5, C1ORF56, HLA-DPB1, RNF167, EMB, SDF4, |
| | | FKRP, SDF2, TM2D1, RYK, ENDOD1, SLAMF7, PMF1, FURIN, BTLA, |
| | | DNASE2, TIGIT, LAMP1, TXNDC12, LAMP2, CD55, APOL1, CD59, |
| | | KIAA0922, PTTGlIP, CD79B, TMEM41A, XCL1, CD200, XCL2, IFI6, CD8A, |
| | | CD8B, FAM3C, HEXB, CLSTN1, PDIA4, DHRSX, ASAH1, PDCD1, |
| | | TMEM109, DGCR2, DNAJC16, CD93, TPP1, RSPRY1, IL4R, GPX7, TM9SF4, |
| | | CCDC107, IFNGR2, LAG3, ICAM1, TM9SF2, C100RF54, SIT1, AIFM1, LY96, |
| | | ICAM3, GZMB, M6PR, DHRS7, IFNAR1, KIAA0100, CD84, TNFRSF10A, |
| | | NUCB1, TOR2A, TNFRSF9, GRM4, CTSL, IFNAR2, C5ORF15, NUS1, |
| | | GINM1, IGF2R, NUCB2, OXNAD1, EMC1, CTSC, FKBP11, CSF2, GPR108, |
| | | TSPEAR, SPOCK2, CSF1, CD247, COLGALT1, APLP2, TNFRSF1A, |
| | | C12ORF49, NUP210, TMED1, SHISA5, TMEM87A, SLC39A6, PCSK7, MR1, |
| | | NENF, CD5, CD27, IL18R1, ALKBH7, IL2RB, CES2, PTPRE, ATRAID, |
| | | PTPRA, LRRN3, CTLA4, C3ORF58, GLA, MCFD2, TAPBPL, POFUT1, |
| | | NCLN, IL2, RCN2 |
| Enrichment Score: - | | |
| 0.0 | | |
| Glycoprotein | 208 | B3GALT6, ADCY7, IL6ST, LHCGR, NELL2, IGHM, TMEM140, IFNG, |
| | | ERAP1, KLRD1, IZUMO4, PLD3, GPR137, BSG, TRABD2A, PIK3IP1, |
| | | ERGIC1, TMEM131, MGAT2, CD37, BTN3A1, NPC2, CAMK4, CD320, CCR4, |
| | | TMEM138, LPAR6, HLA-DPA1, CD226, BTN3A2, SUCO, GLG1, ORAI1, |
| | | CLCN3, TMX3, OAS2, SLC11A2, SERINC3, LIF, SLC29A1, SYPL1, |
| | | SERINC1, RAC1, HLA-DPB1, EMB, MGAT4A, GPR155, ST6GAL1, TM2D1, |
| | | SLAMF7, CD63, BTLA, CD55, APOL1, ATP13A1, CD59, KIAA0922, |
| | | TMEM41A, CD200, ENOX2, SLC20A2, AP1AR, CD151, ASAH1, LNPEP, |
| | | DNAJC16, IL4R, CLDND1, DPP4, LAG3, ICAM1, C10ORF54, LY96, ICAM3, |
| | | GZMB, TNFRSF10A, GRM4, MTMR14, NUS1, CHSY1, CSF2, CSF1, ABHD2, |
| | | COLGALT1, NUP214, NUMA1, STT3A, GNPTAB, C12ORF49, NUP210, |
| | | PCSK7, CD27, IL18R1, CES2, LRRN3, CTLA4, TMEM2, P2RY10, GLA, |
| | | NCLN, IL2, TSPAN5, ST3GAL1, GLT8D1, RNF149, SPN, TOR1AIP2, |
| | | TOR1AIP1, STIM1, MOGS, HCST, PNPLA8, TMEM106B, IGSF8, LRP10, |
| | | MFAP3, UGGT1, TMEM214, ITGAE, CERS6, UNC93B1, TMEM219, CTSA, |
| | | TRDC, ITGB1, SRF, P2RY8, CERS2, ENTPD6, RNF167, B3GNT2, FKRP, |
| | | SDF4, TMEM30A, LYSMD3, B4GALT3, YIPF6, RYK, FURIN, ATF6, |
| | | DNASE2, TIGIT, LAMP1, LAMP2, PTTGlIP, CD79B, LRRC8B, CD8B, |
| | | HEXB, CLSTN1, MFSD2A, CXCR3, PDCD1, DGCR2, CD93, TPP1, RSPRY1, |
| | | CXCR4, IFNGR2, AKT2, SIT1, PFKL, PIGS, M6PR, TMEM245, MCM6, |
| | | IFNAR1, KIAA0100, CD84, NUCB1, TOR2A, TNFRSF9, C5ORF15, IFNAR2, |
| | | CTSL, TNFSF13B, CLECL1, GINM1, IGF2R, EMC1, CTSC, C16ORF54, |
| | | GPR108, FAM173A, TSPEAR, SPOCK2, TPRA1, TMEM259, APLP2, |
| | | TNFRSF1A, HRH2, TSPAN31, TMEM87A, SLC39A6, MR1, APMAP, CD5, |
| | | IL2RB, PTPRE, ATRAID, PTPRA, SUN2, NUP155, TSPAN17, CLPTM1, SP1, |
| | | SLC6A6, POFUT1 |
| signal peptide | 165 | ATP6AP2, IL6ST, NELL2, LHCGR, SLC52A2, IGHM, SMIM7, IFNG, RNF149, |
| | | DDOST, IZUMO4, SPN, GPR137, BSG, PMCH, TRABD2A, STIM1, PIK3IP1, |
| | | CECR5, HCST, IGSF8, ABHD17B, BTN3A1, CHID1, NPC2, LRP10, CD320, |
| | | HLA-DPA1, KDSR, MFAP3, AMFR, CD226, UGGT1, BTN3A2, SUCO, |
| | | HSD17B11, GLG1, TMX2, CCL3, C16ORF91, ITGAE, TMX3, CTSA, CCL4, |
| | | ITGB1, CASD1, LIF, BL0C155, C1ORF56, HLA-DPB1, RNF167, EMB, SDF4, |
| | | FKRP, SDF2, TM2D1, RYK, ENDOD1, SLAMF7, PMF1, FURIN, BTLA, |
| | | DNASE2, TIGIT, LAMP1, TXNDC12, LAMP2, CD55, APOL1, CD59, |
| | | KIAA0922, PTTGlIP, CD79B, TMEM41A, XCL1, CD200, XCL2, IFI6, CD8A, |
| | | CD8B, FAM3C, HEXB, CLSTN1, PDIA4, DHRSX, ASAH1, PDCD1, |
| | | TMEM109, DGCR2, DNAJC16, CD93, TPP1, RSPRY1, IL4R, GPX7, TM9SF4, |
| | | CCDC107, IFNGR2, LAG3, ICAM1, TM9SF2, C10ORF54, SIT1, AIFM1, LY96, |
| | | ICAM3, GZMB, M6PR, DHRS7, IFNAR1, KIAA0100, CD84, TNFRSF10A, |
| | | NUCB1, TOR2A, TNFRSF9, GRM4, CTSL, IFNAR2, C5ORF15, NUS1, |
| | | GINM1, IGF2R, NUCB2, OXNAD1, EMC1, CTSC, FKBP11, CSF2, GPR108, |
| | | TSPEAR, SPOCK2, CSF1, CD247, COLGALT1, APLP2, TNFRSF1A, |
| | | C120RF49, NUP210, TMED1, SHISA5, TMEM87A, SLC39A6, PCSK7, MR1, |
| | | NENF, CD5, CD27, IL18R1, ALKBH7, IL2RB, CES2, PTPRE, ATRAID, |
| | | PTPRA, LRRN3, CTLA4, C3ORF58, GLA, MCFD2, TAPBPL, POFUT1, |
| | | NCLN, IL2, RCN2 |
| glycosylation site: N- | 189 | ADCY7, B3GALT6, IL6ST, NELL2, TSPAN5, LHCGR, IGHM, TMEM140, |
| linked (GlcNAc . . . ) | | ST3GAL1, GLT8D1, IFNG, ERAP1, RNF149, KLRD1, IZUM04, SPN, |
| | | TOR1AIP2, PLD3, GPR137, BSG, TRABD2A, TOR1AIP1, STIM1, MOGS, |
| | | ERGIC1, TMEM131, MGAT2, PNPLA8, TMEM106B, BTN3A1, CD37, IGSF8, |
| | | NPC2, LRP10, CD320, CCR4, LPAR6, TMEM138, HLA-DPA1, MFAP3, |
| | | AMFR, BTN3A2, CD226, UGGT1, SUCO, GLG1, ORAI1, CLCN3, TMEM214, |
| | | ITGAE, TMX3, TMEM63A, CERS6, UNC93B1, TMEM219, CTSA, ITGB1, |
| | | SLC29A1, SERINC3, SLC11A2, P2RY8, LIF, SYPL1, CERS2, SERINC1, |
| | | ENTPD6, HLA-DPB1, EMB, RNF167, B3GNT2, FKRP, SDF4, TMEM30A, |
| | | LYSMD3, MGAT4A, GPR155, ST6GAL1, B4GALT3, YIPF6, TM2D1, RYK, |
| | | SLAMF7, CD63, FURIN, BTLA, ATF6, DNASE2, TIGIT, LAMP 1, LAMP2, |
| | | CD55, APOL1, ATP13A1, CD59, KIAA0922, PTTGlIP, CD79B, TMEM41A, |
| | | CD200, LRRC8B, CD8B, SLC20A2, AP1AR, HEXB, LRRC8D, CLSTN1, |
| | | CXCR3, CD151, ASAH1, PDCD1, LNPEP, DGCR2, DNAJC16, CD93, TPP1, |
| | | RSPRY1, CXCR4, IL4R, CLDND1, IFNGR2, LAG3, DPP4, ICAM1, |
| | | C100RF54, SIT1, LY96, ICAM3, PIGS, GZMB, TMEM245, M6PR, IFNAR1, |
| | | KIAA0100, CD84, TNFRSF10A, TOR2A, TNFRSF9, GRM4, CTSL, IFNAR2, |
| | | C5ORF15, MTMR14, CLECL1, TNFSF13B, GINM1, IGF2R, EMC1, CHSY1, |
| | | CTSC, CSF2, GPR108, FAM173A, TSPEAR, SPOCK2, CSF1, TPRA1, |
| | | TMEM259, COLGALT1, TNFRSF1A, STT3A, GNPTAB, C120RF49, HRH2, |
| | | NUP210, TSPAN31, TMEM87A, SLC39A6, PCSK7, MR1, APMAP, CD5, |
| | | CD27, IL18R1, IL2RB, CES2, PTPRE, ATRAID, PTPRA, LRRN3, CTLA4, |
| | | SUN2, TSPAN17, TMEM2, CLPTM1, P2RY10, GLA, SLC6A6, POFUT1, |
| | | NCLN |
| Signal | 235 | CDIPT, TMEM19, SEC31A, IL6ST, LHCGR, NELL2, MPV17, SLC52A2, |
| | | HIBADH, SHKBP1, PGP, IFNG, ERAP1, IZUMO4, TIMMDC1, BSG, CAPNS1, |
| | | PMCH, TRABD2A, UBR2, PIK3IP1, TMEM131, BTN3A1, NPC2, CD320, |
| | | HLA-DPA1, FAM177A1, CD226, BTN3A2, SUCO, TMX2, HSD17B11, GLG1, |
| | | C16ORF91, TMX3, NPIPB4, AKAP10, LIF, C1ORF56, RAC1, HLA-DPB1, |
| | | EMB, GPR155, LAPTM4A, TM2D1, SLAMF7, BTLA, APOL3, CD55, APOL1, |
| | | ATP13A1, CD59, KIAA0922, TMEM41A, CD200, METTL17, FAM96A, |
| | | FAM3C, VPS37B, PDIA4, ASAH1, TMEM109, DNAJC16, IL4R, GPX7, |
| | | CLDND1, LAG3, ICAM1, C10ORF54, LY96, ICAM3, GZMB, ECSIT, |
| | | TNFRSF10A, GRM4, MTMR14, FKBP11, CSF2, EXOC7, USP3, CSF1, |
| | | COLGALT1, DNAJC30, STAU1, NUP214, C12ORF49, NUP210, PCSK7, |
| | | NENF, CD27, IL18R1, CES2, LRRN3, CTLA4, DNPEP, SDHA, FAM78A, |
| | | GLA, MCFD2, JMJD8, PHGDH, TAPBPL, OGG1, NCLN, GOLGB1, IL2, |
| | | RALY, TSPO, ATP6AP2, HBS1L, RGL4, SMIM7, RNF149, DDOST, SPN, |
| | | TMEM205, STIM1, CECR5, HCST, RALGAPA1, IGSF8, ABHD17B, CHID1, |
| | | LRP10, KDSR, MFAP3, SMARCA2, UGGT1, PCCB, CCL3, ITGAE, |
| | | TMEM219, MUM1, CTSA, ITGB1, CCL4, CASD1, FBXW7, C12ORF10, |
| | | RNF167, SDF4, SDF2, RYK, C21ORF33, ENDOD1, ABCB7, FURIN, DNASE2, |
| | | TIGIT, TXNDC12, LAMP1, LAMP2, TXNDC11, DNAJB9, PDE7A, PTTGlIP, |
| | | CD79B, XCL1, XCL2, IF16, CD8A, CD8B, YLPM1, HEXI3, CLSTN1, DHRSX, |
| | | LSR, ZZEF1, PDCD1, DGCR2, CD93, RSPRY1, TPP1, CCDC107, TM9SF4, |
| | | IFNGR2, EBAG9, TM9SF2, ELP2, CCDC88B, SIT1, DYNLT3, M6PR, |
| | | IFNAR1, DHRS7, KIAA0100, NUCB1, CD84, TOR2A, CTSL, C5ORF15, |
| | | TNFRSF9, IFNAR2, PANK2, HIPK1, GINM1, IGF2R, CD81, HIPK2, DDT, |
| | | NUCB2, OXNAD1, CTSC, EMC1, KPNA1, C7ORF73, GPR108, TSPEAR, |
| | | SPOCK2, CD247, APLP2, TNFRSF1A, NUDT9, TSPAN31, TMED1, SHISA5, |
| | | TMEM87A, SLC39A6, MR1, CD5, IL2RB, PTPRE, ATRAID, PTPRA, CBL, |
| | | RAF1, SLC10A3, GORAB, WSB1, ADI1, C3ORF58, TDP2, SLC6A6, POFUT1, |
| | | RCN2 |
| topological | 213 | B3GALT6, ADCY7, VAPB, IL6ST, LHCGR, USE1, TMEM140, ATP2B4, |
| domain: Cytoplasmic | | ERAP1, JAGN1, KLRD1, PLD3, GPR137, BSG, TRABD2A, UBE2J1, UBE2J2, |
| | | PIK3IP1, ERGIC1, MGAT1, MGAT2, CD37, BTN3A1, CD320, ATP2C1, CCR4, |
| | | LPAR6, HLA-DPA1, AMFR, CD226, BTN3A2, GLG1, TMX2, ORAIl, CLCN3, |
| | | C16ORF91, TMX3, SLC11A2, SERINC3, SLC29A1, SYPL1, STX17, SERINC1, |
| | | HLA-DPB1, EMB, STX10, MGAT4A, ST6GAL1, SPTSSA, ATP11A, SLAMF7, |
| | | CD63, BTLA, ATP13A1, SYNE2, KIAA0922, BET1L, CD200, RTN4, |
| | | SLC20A2, CD151, HVCN1, LNPEP, DNAJC16, IL4R, SLMAP, LAG3, DPP4, |
| | | ICAM1, C10ORF54, ZDHHC3, JKAMP, ZDHHC8, ICAM3, TNFRSF10A, |
| | | GRM4, ZDHHC16, BNIP1, NUS1, LRMP, CHSY1, CSF1, BET1, SFT2D2, |
| | | STT3A, NUP210, PEX16, PCSK7, ACSL4, ACSL3, C1GALT1, CD27, ACSL5, |
| | | IL18R1, EPB41L4A-AS1, LRRN3, CTLA4, TMEM5, ITPR2, SAMD8, PLSCR1, |
| | | P2RY10, TAPBPL, NCLN, GOLGB1, ATP6AP2, UNC50, TSPAN5, SLC7A6, |
| | | ST3GAL1, GLT8D1, STARD3, MFF, SMIM7, LRRC59, MS4A1, DDOST, SPN, |
| | | SCAMP3, STIM1, MOGS, HCST, BCAP31, IGSF8, LRP10, VAMPS, KDSR, |
| | | VAMP2, MFAP3, DERL1, ITGAE, CERS6, ITGB1, PRR7, P2RY8, FIS1, |
| | | CERS2, ENTPD6, SLC30A5, B3GNT2, LYSMD3, PRAF2, B4GALT3, |
| | | TMEM222, RYK, SYT11, NIPA2, SREBF2, ATF6, TIGIT, LAMP1, LAMP2, |
| | | TRAF3IP3, PTTGlIP, RHOT1, RHOT2, CD79B, CD8A, CD8B, ATL3, |
| | | CLSTN1, PEX3, CXCR3, LSR, PDCD1, DGCR2, CD93, CXCR4, ATP8B2, |
| | | IFNGR2, EBAG9, TM9SF2, SIT1, PIGS, M6PR, LDLRAD4, IFNAR1, CD84, |
| | | TNFRSF9, C5ORF15, IFNAR2, TNFSF13B, CLECL1, GINM1, IGF2R, CD81, |
| | | EMC1, GALNT2, CD247, ALDH3A2, APLP2, NDC1, TNFRSF1A, HRH2, |
| | | TSPAN31, TMED1, SHISA5, SEC22B, SLC39A6, MR1, APMAP, SLC39A3, |
| | | CD5, IL2RB, PTPRE, GIMAP5, ATRAID, PTPRA, TSPAN17, GIMAP1, |
| | | CLPTM1, LMBR1L, SLC6A6 |
| topological | 130 | IL6ST, ATP6AP2, LHCGR, TSPAN5, SLC7A6, TMEM140, STARD3, ATP2B4, |
| domain: Extracellular | | SMIM7, KLRD1, SPN, GPR137, BSG, TRABD2A, STIM1, PIK3IP1, HCST, |
| | | IGSF8, CD37, BTN3A1, CD320, LRP10, LPAR6, CCR4, HLA-DPA1, AMFR, |
| | | MFAP3, CD226, BTN3A2, GLG1, TMX2, ORAI1, C16ORF91, ITGAE, ITGB1, |
| | | P2RY8, SERINC3, SLC11A2, SLC29A1, PRR7, SERINC1, SLC30A5, HLA- |
| | | DPB1, EMB, LYSMD3, PRAF2, TMEM222, RYK, ATP11A, NIPA2, SLAMF7, |
| | | CD63, BTLA, TIGIT, ATP13A1, KIAA0922, TRAF3IP3, PTTGlIP, CD79B, |
| | | CD200, CD8A, SLC20A2, CD8B, CLSTN1, CXCR3, CD151, HVCN1, LSR, |
| | | PDCD1, LNPEP, DGCR2, DNAJC16, CD93, CXCR4, IL4R, SLMAP, ATP8B2, |
| | | IFNGR2, LAG3, DPP4, EBAG9, ICAM1, C100RF54, SIT1, ICAM3, |
| | | LDLRAD4, IFNAR1, CD84, TNFRSF10A, TNFRSF9, GRM4, C5ORF15, |
| | | IFNAR2, NUS1, TNFSF13B, CLECL1, GINM1, CD81, EMC1, CD247, APLP2, |
| | | TNFRSF1A, HRH2, TSPAN31, TMED1, SHISA5, SLC39A6, PCSK7, APMAP, |
| | | MR1, SLC39A3, CD5, CD27, IL18R1, IL2RB, PTPRE, GIMAP5, ATRAID, |
| | | PTPRA, LRRN3, CTLA4, TMEM5, TSPAN17, GIMAP1, ITPR2, CLPTM1, |
| | | LMBR1L, PLSCR1, P2RY10, SLC6A6 |

### Example 2. Human-Derived Macrophages Infected with M. tuberculosis

Tuberculosis (TB) is an infectious disease caused by bacterium Mycobacterium tuberculosis (MTB). Currently, TB remains one of the most significant infectious disease worldwide, causing 1.8 million deaths annually (WHO. Global tuberculosis report 2016). MTB preferably infects macrophages in lungs, replicates within invaded macrophages, and induces cytokines that initiate inflammatory responses, resulting in granuloma in lungs (and potentially other organs). In MTB infected patients, infectious states and symptoms can vary greatly within the same organ or even tissues. In the same infected lobe, granuloma states can range from resolved to cavitary lesion and caseating.

The standard tests to detect active TB include detecting lung lesion with X-ray images and MTB specific antibody detection. Previous studies have identified biomarkers for diagnosis of active TB and to monitor TB therapy efficiency. However, these biomarkers rely on limited numbers of antigen recognition, or suffer from insensitivity under certain conditions (Friedrich S O et al., Lancet Respir Med. 2013 August; 1(6):462-70; Coppola et al., Tuberculosis 2017, 106: 25-32). A sensitive test that allows for diagnosis with easily obtainable specimens and one that responds to MTB load largely remains in need (Goletti et al., Infectious Disease Reports 2016; 8(2): 6568). More importantly, a comprehensive understanding of immune response and pathogenesis in different states of TB infection is still missing. Thus, it remains unclear whether intrinsic factors or extrinsic micro-environment, or both contribute to the different behavior of infected macrophages at these different states and how macrophage phenotype is related to the quantity of intracellular MTB (particularly, MTB multiplicity of infection, MOI).

Human monocytes derived macrophages are infected with an Mycobatcterium tuberculosis expressing RFP for ˜72 hours. Single cells are collected and sorted into 96 well plates using a tet-reporter system in the bacteria by flow cytometry. MTB infected cells indexed with fluorescence intensity of intracellular bacteria as well as uninfected bystanders were collected and single cell RNA-Sequencing was performed.

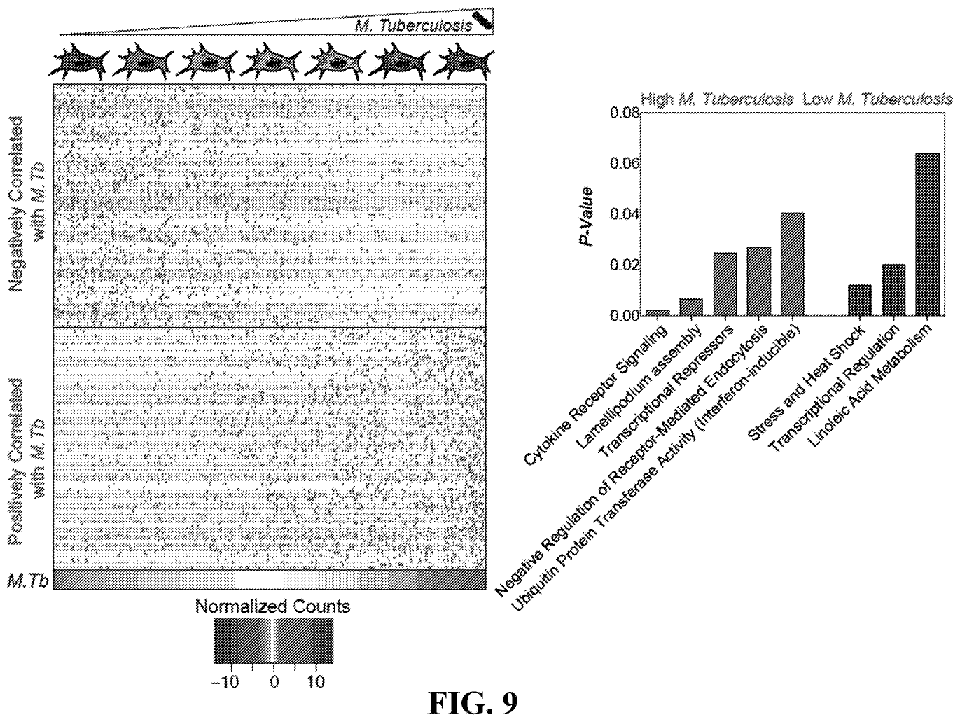

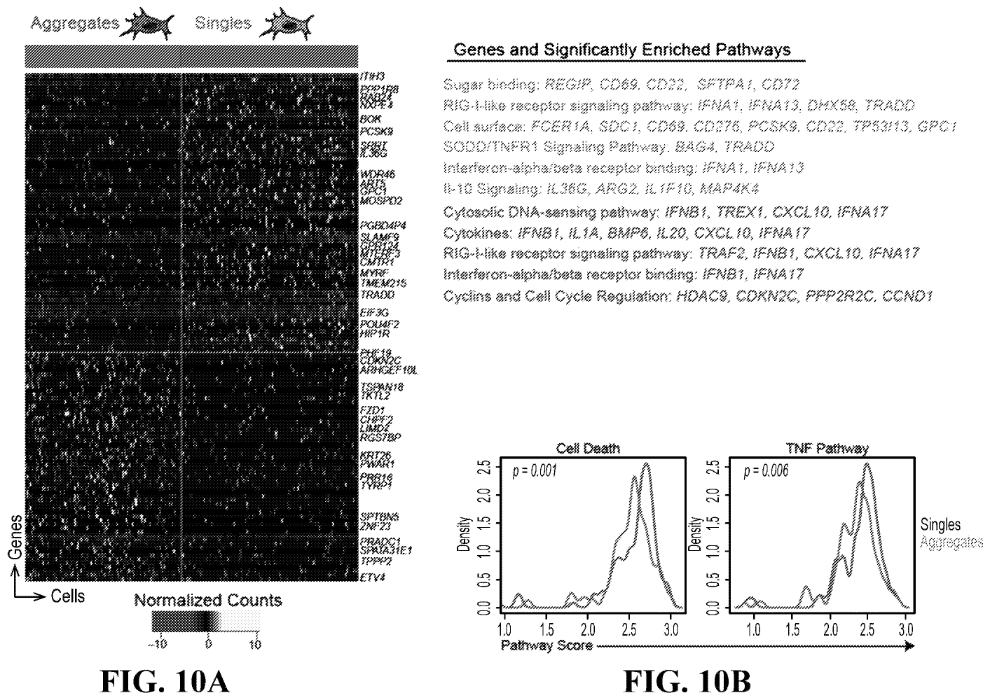

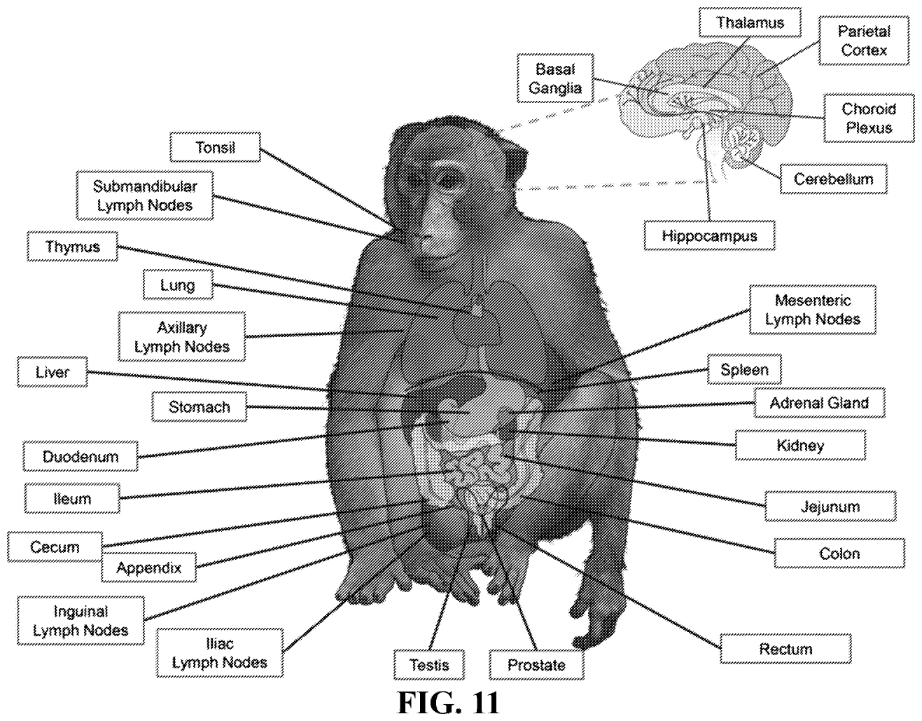

Based on single cell whole transcriptome amplification, genes whose expression are positively (Table 4) or negatively (Table 5) correlated with MTB infection have been identified. The correlated genes are clustered according to their function(s) (Tables 4 and 5). Further, genes and pathways that are correlated with low MOI (FIG. 8 E, top, FIGS. 9-10) and high MOI (FIG. 8E, bottom, FIGS. 9-10) are also identified. Particularly, the TNFR2 pathway, NOS1 pathway, ETS pathway, NTHI pathway, IL1R pathway, NFkB pathway, MAL pathway, ALK pathway, RAC1 pathway, CCR3 pathway, GCR pathway, PPAR signaling pathway, PPARA activation of gene expression, PPARA pathway, amyotrophic lateral sclerosis pathway, activation of BH3 proteins, SNARE interaction for vesicular transport, Golgi associated vesicle biogenesis, TGF beta signaling pathway, TGF beta receptor signaling activates SMADS, signaling by TGF beta receptor complex, Hematopietic cell lineage, leishmanial infection, glycosaminoglycan biosynthesis and chondroitin sulfate biosynthesis, metabolism of vitamins and cofactors, fatty Acyl CoA biosynthesis, synthesis, secretion, and inactivation of GLP1, incretin synthesis, secretion, and inactivation, fatty acid triacylglycerol and ketone body metabolism, glycosaminoglycan metabolism, triglyceride biosynthesis, cytokine receptor signaling, lamellipodium assembly, transcriptional repressors, negative regulation of receptor mediated endocytosis, and ubiquitin protein transfer activity, IFNB1, TREX1, CXCL10, IFNA17 in cytosolic DNA sensing pathway, HDAC9, CDKN2C, PPP2R2C, CCND1 in cicins and cell cycle regulation appear to correlate with high MTB MOI. The PARRXR pathway, transcription, RNA Pol I/III Transcription, PI3k Events in ERBB2 signaling, signaling by ERBB2, signaling by EGFR in cancer, chromosome maintenance, telomere maintenance, Acyl chain remodeling, N glycan biosynthesis and protein transfer, respiratory electron transport, chemiosmotic coupling, glycine, serine, and threonine metabolism, TCA cycle, glycolysis and gluconeogenesis, phenylalanine metabolism, purine metabolism, stress and heat shock, transcriptional regulation, linoleic acid metabolism, REGIP, CD69, CD22, SFTPA1, CD72 involved in sugar binding, IFNA1, IFNA13, DHX58, TRADD in RIG-I-like receptor singling pathway, FCER1A, SDC1, CD69, CD276, PCSK9, CD22, TP53I13, GPC1 in cell surface recognition, BAG4 in SODD/TNFR1 signaling pathway, IFNA1 and IFNA13 interferon alpha/beta receptor binding; IL36 G, ARG2, IL1F10, MAP4K4 in Il-10 signaling in appear to correlate with low MTB MOI (FIGS. 8-10).

TABLE 4
Genes Positively Correlated with TB

| | | |
|---|---|---|
| Term | Count | Genes |

Enrichment Score: 1.6292421548153069

| | | |
|---|---|---|
| GO:0045806~negative regulation | 3 | RAC1, APOC1, NR1H3 |
| of endocytosis | | |
| GO:0060627~regulation of vesicle- | 4 | RAB3C, RAC1, APOC1, NR1H3 |
| mediated transport | | |
| GO:0045834~positive regulation of | 3 | RAC1, APOC1, NR1H3 |
| lipid metabolic process | | |
| GO:0030100~regulation of | 3 | RAC1, APOC1, NR1H3 |
| endocytosis | | |
| GO:0019216~regulation of lipid | 3 | RAC1, APOC1, NR1H3 |
| metabolic process | | |
| GO:0051051~negative regulation of | 3 | RAC1, APOC1, NR1H3 |
| transport | | |
| GO:0051129~negative regulation of | 3 | RAC1, APOC1, NR1H3 |
| cellular component organization | | |

Enrichment Score: 1.0362232095437491

| | | |
|---|---|---|
| GO:0065003~macromolecular | 8 | TARBP2, TSPYL2, MED27, RAC1, APOC1, H2AFY, NAP1L3, |
| complex assembly | | ZW10 |
| GO:0043933~macromolecular | 8 | TARBP2, TSPYL2, MED27, RAC1, APOC1, H2AFY, NAP1L3, |
| complex subunit organization | | ZW10 |
| GO:0034622~cellular | 5 | TARBP2, TSPYL2, RAC1, H2AFY, NAP1L3 |
| macromolecular complex assembly | | |
| GO:0006334~nucleosome assembly | 3 | TSPYL2, H2AFY, NAP1L3 |
| GO:0031497~chromatin assembly | 3 | TSPYL2, H2AFY, NAP1L3 |
| GO:0065004-protein-DNA complex | 3 | TSPYL2, H2AFY, NAP1L3 |
| assembly | | |
| GO:0034728-nucleosome | 3 | TSPYL2, H2AFY, NAP1L3 |
| organization | | |
| GO:0034621-cellular | 5 | TARBP2, TSPYL2, RAC1, H2AFY, NAP1L3 |
| macromolecular complex subunit | | |
| organization | | |
| GO:0006323~DNA packaging | 3 | TSPYL2, H2AFY, NAP1L3 |
| GO:0006333~chromatin assembly | 3 | TSPYL2, H2AFY, NAP1L3 |
| or disassembly | | |
| GO:0051276~chromosome | 5 | TSPYL2, H2AFY, NAP1L3, RNF20, ZW10 |
| organization | | |
| chromatin regulator | 3 | TSPYL2, H2AFY, RNF20 |
| GO:0006325~chromatin | 4 | TSPYL2, H2AFY, NAP1L3, RNF20 |
| organization | | |
| GO:0016568~chromatin | 3 | TSPYL2, H2AFY, RNF20 |
| modification | | |
| GO:0006461~protein complex | 3 | MED27, RAC1, ZW10 |
| assembly | | |
| GO:0070271~protein complex | 3 | MED27, RAC1, ZW10 |
| biogenesis | | |

Enrichment Score: 0.9651359055052946

| | | |
|---|---|---|
| ubl conjugation pathway | 6 | HERC6, UCHL5, KLHL12, TRIM63, KLHL3, RNF20 |
| GO:0006508~proteolysis | 9 | CAPN11, HERC6, UCHL5, KLHL12, MMP16, TRIM63, |
| | | KLHL3, RNF20, DHCR24 |
| GO:0043632~modification- | 6 | HERC6, UCHL5, KLHL12, TRIM63, KLHL3, RNF20 |
| dependent macromolecule catabolic | | |
| process | | |
| GO:0019941~modification- | 6 | HERC6, UCHL5, KLHL12, TRIM63, KLHL3, RNF20 |
| dependent protein catabolic process | | |
| GO:0051603~proteolysis involved | 6 | HERC6, UCHL5, KLHL12, TRIM63, KLHL3, RNF20 |
| in cellular protein catabolic | | |
| process | | |
| GO:0009057-macromolecule | 7 | HERC6, UCHL5, KLHL12, TRIM63, KLHL3, RNF20, DHCR24 |
| catabolic process | | |
| ligase | 4 | HERC6, ACSL4, TRIM63, RNF20 |
| GO:0044257~cellular protein | 6 | HERC6, UCHL5, KLHL12, TRIM63, KLHL3, RNF20 |
| catabolic process | | |
| GO:0030163~protein catabolic | 6 | HERC6, UCHL5, KLHL12, TRIM63, KLHL3, RNF20 |
| process | | |
| GO:0044265~cellular | 6 | HERC6, UCHL5, KLHL12, TRIM63, KLHL3, RNF20 |
| macromolecule catabolic process | | |
| GO:0016881~acid-amino acid ligase | 3 | HERC6, TRIM63, RNF20 |
| activity | | |
| GO:0016879~ligase activity, | 3 | HERC6, TRIM63, RNF20 |
| forming carbon-nitrogen bonds | | |
| GO:0006511~ubiquitin-dependent | 3 | UCHL5, TRIM63, RNF20 |
| protein catabolic process | | |

Enrichment Score: 0.8195579938188856

| | | |
|---|---|---|
| hsa04010:MAPK signaling pathway | 4 | DUSP1, RAC1, PPP3R1, IL1A |
| GO:0006915~apoptosis | 6 | RAC1, PPP3R1, LGALS12, IL1A, DHCR24, FGD4 |
| GO:0043065~positive regulation | 5 | DUSP1, RAC1, PPP3R1, LGALS12, FGD4 |
| of apoptosis | | |
| GO:0043068~positive regulation of | 5 | DUSP1, RAC1, PPP3R1, LGALS12, FGD4 |
| programmed cell death | | |
| GO:0010942~positive regulation of | 5 | DUSP1, RAC1, PPP3R1, LGALS12, FGD4 |
| cell death | | |
| GO:0012501~programmed cell | 6 | RAC1, PPP3R1, LGALS12, IL1A, DHCR24, FGD4 |
| death | | |
| GO:0042981~regulation of | 7 | DUSP1, RAC1, PPP3R1, LGALS12, IL1A, DHCR24, FGD4 |
| apoptosis | | |
| GO:0043067~regulation of | 7 | DUSP1, RAC1, PPP3R1, LGALS12, IL1A, DHCR24, FGD4 |
| programmed cell death | | |
| GO:0010941~regulation of cell | 7 | DUSP1, RAC1, PPP3R1, LGALS12, IL1A, DHCR24, FGD4 |
| death | | |
| GO:0008219~cell death | 6 | RAC1, PPP3R1, LGALS12, IL1A, DHCR24, FGD4 |
| GO:0016265~death | 6 | RAC1, PPP3R1, LGALS12, IL1A, DHCR24, FGD4 |
| GO:0019899~enzyme binding | 4 | RAC1, RNF20, DHCR24, FGD4 |
| GO:0006917~induction of | 3 | RAC1, LGALS12, FGD4 |
| apoptosis | | |
| GO:0012502~induction of | 3 | RAC1, LGALS12, FGD4 |
| programmed cell death | | |
| GO:0005829~cytosol | 6 | RAB3C, APLF, HERC6, RAC1, PPP3R1, FGD4 |

Enrichment Score: 0.7891679915209755

| | | |
|---|---|---|
| lipoprotein | 7 | RAB3C, RAPSN, RAC1, APOC1, PPP3R1, RHOD, IL1A |
| GO:0009898~internal side of | 5 | VEPH1, RAB3C, RAPSN, RAC1, RHOD |
| plasma membrane | | |
| short sequence motif:Effector region | 3 | RAB3C, RAC1, RHOD |
| lipid moiety-binding region:S- | 3 | RAB3C, RAC1, RHOD |
| geranylgeranyl cysteine | | |
| methylation | 4 | RAB3C, RAC1, H2AFY, RHOD |
| IPR013753:Ras | 3 | RAB3C, RAC1, RHOD |
| nucleotide phosphate-binding | 4 | RAB3C, RAC1, RHOD, RRAGD |
| region:GTP | | |
| IPR001806:Ras GTPase | 3 | RAB3C, RAC1, RHOD |
| prenylation | 3 | RAB3C, RAC1, RHOD |
| gtp-binding | 4 | RAB3C, RAC1, RHOD, RRAGD |
| GO:0007264~small GTPase | 4 | RAB3C, RAC1, RHOD, DHCR24 |
| mediated signal transduction | | |
| IPR005225:Small GTP-binding | 3 | RAB3C, RAC1, RHOD |
| protein | | |
| GO:0005525~GTP binding | 4 | RAB3C, RAC1, RHOD, RRAGD |
| GO:0019001~guanyl nucleotide | 4 | RAB3C, RAC1, RHOD, RRAGD |
| binding | | |
| GO:0032561~guanyl ribonucleotide | 4 | RAB3C, RAC1, RHOD, RRAGD |
| binding | | |
| GO:0007242~intracellular signaling | 7 | RAB3C, DUSP1, RAC1, RHOD, DHCR24, MTNR1A, FGD4 |
| cascade | | |
| nucleotide-binding | 6 | RAB3C, APLF, RAC1, RHOD, ACSL4, RRAGD |
| GO:0017076~purine nucleotide | 7 | RAB3C, RAC1, ZNF12, RHOD, ACSL4, RRAGD, DHCR24 |
| binding | | |
| GO:0000166~nucleotide binding | 8 | RAB3C, APLF, RAC1, ZNF12, RHOD, ACSL4, RRAGD, |
| | | DHCR24 |
| GO:0032553~ribonucleotide | 6 | RAB3C, RAC1, ZNF12, RHOD, ACSL4, RRAGD |
| binding | | |
| GO:0032555~purine ribonucleotide | 6 | RAB3C, RAC1, ZNF12, RHOD, ACSL4, RRAGD |
| binding | | |

Enrichment Score: 0.7538123661108601

| | | |
|---|---|---|
| GO:0034622~cellular | 5 | TARBP2, TSPYL2, RAC1, H2AFY, NAP1L3 |
| macromolecular complex assembly | | |
| GO:0034621~cellular | 5 | TARBP2, TSPYL2, RAC1, H2AFY, NAP1L3 |
| macromolecular complex subunit | | |
| organization | | |
| GO:0043549~regulation of kinase | 4 | TARBP2, TSPYL2, RAC1, FGD4 |
| activity | | |
| GO:0051338~regulation of | 4 | TARBP2, TSPYL2, RAC1, FGD4 |
| transferase activity | | |
| GO:0042325~regulation of | 4 | TARBP2, TSPYL2, RAC1, FGD4 |
| phosphorylation | | |
| GO:0019220~regulation of | 4 | TARBP2, TSPYL2, RAC1, FGD4 |
| phosphate metabolic process | | |
| GO:0051174~regulation of | 4 | TARBP2, TSPYL2, RAC1, FGD4 |
| phosphorus metabolic process | | |
| GO:0045859~regulation of protein | 3 | TARBP2, TSPYL2, FGD4 |
| kinase activity | | |

Enrichment Score: 0.7376548894910318

| | | |
|---|---|---|
| zinc-finger | 14 | PRDM8, ASXL3, SNAI3, APLF, RAPSN, ZDHHC7, GLIS1, |
| | | ZNF24, ZNF12, HNF4G, TRIM63, RNF20, FGD4, NR1H3 |
| zinc | 15 | PRDM8, ASXL3, SNAI3, APLF, RAPSN, ZDHHC7, GLIS1, |
| | | ZNF24, ZNF12, MMP16, HNF4G, TRIM63, RNF20, FGD4, |
| | | NR1H3 |
| GO:0008270~zinc ion binding | 15 | PRDM8, ASXL3, SNAI3, APLF, RAPSN, ZDHHC7, GLIS1, |
| | | ZNF24, ZNF12, MMP16, HNF4G, TRIM63, RNF20, FGD4, |
| | | NR1H3 |
| repressor | 5 | ASXL3, SNAI3, GLIS1, ZNF24, MSC |
| nucleus | 22 | ASXL3, APLF, GLIS1, LGALS12, ZNF24, ZNF12, NAP1L3, |
| | | HNF4G, TRIM63, RRAGD, MSC, NOP10, PRDM8, TARBP2, |
| | | SNAI3, MEIS2, TSPYL2, DUSP26, MED27, H2AFY, RNF20, |
| | | NR1H3 |
| GO:0043169~cation binding | 22 | ASXL3, PCDHGA10, APLF, CRTAC1, TRPV2, ZDHHC7, |
| | | GLIS1, APOC1, PPP3R1, ZNF24, ZNF12, MMP16, HNF4G, |
| | | TRIM63, PRDM8, SNAI3, CAPN11, RAPSN, ACSL4, RNF20, |
| | | NR1H3, FGD4 |
| GO:0043167~ion binding | 22 | ASXL3, PCDHGA10, APLF, CRTAC1, TRPV2, ZDHHC7, |
| | | GLIS1, APOC1, PPP3R1, ZNF24, ZNF12, MMP16, HNF4G, |
| | | TRIM63, PRDM8, SNAI3, CAPN11, RAPSN, ACSL4, RNF20, |
| | | NR1H3, FGD4 |
| metal-binding | 15 | PRDM8, ASXL3, SNAI3, APLF, RAPSN, ZDHHC7, GLIS1, |
| | | ZNF24, ZNF12, MMP16, HNF4G, TRIM63, RNF20, FGD4, |
| | | NR1H3 |
| transcription regulation | 11 | PRDM8, ASXL3, SNAI3, TSPYL2, GLIS1, MED27, ZNF24, |
| | | ZNF12, HNF4G, MSC, NR1H3 |
| zinc finger region:C2H2-type 3 | 5 | PRDM8, SNAI3, GLIS1, ZNF24, ZNF12 |
| GO:0046872~metal ion binding | 21 | ASXL3, PCDHGA10, APLF, CRTAC1, TRPV2, ZDHHC7, |
| | | GLIS1, PPP3R1, ZNF24, ZNF12, MMP16, HNF4G, TRIM63, |
| | | PRDM8, SNAI3, CAPN11, RAPSN, ACSL4, RNF20, NR1H3, |
| | | FGD4 |
| Transcription | 11 | PRDM8, ASXL3, SNAI3, TSPYL2, GLIS1, MED27, ZNF24, |
| | | ZNF12, HNF4G, MSC, NR1H3 |
| IPR007087:Zinc finger, C2H2-type | 6 | PRDM8, SNAI3, GLIS1, ZNF24, ZNF12, NR1H3 |
| GO:0046914~transition metal ion | 15 | PRDM8, ASXL3, SNAI3, APLF, RAPSN, ZDHHC7, GLIS1, |
| binding | | ZNF24, ZNF12, MMP16, HNF4G, TRIM63, RNF20, FGD4, |
| | | NR1H3 |
| dna-binding | 10 | PRDM8, SNAI3, MEIS2, GLIS1, ZNF24, H2AFY, ZNF12, |
| | | HNF4G, MSC, NR1H3 |
| GO:0045449~regulation of | 14 | ASXL3, GLIS1, ZNF24, ZNF12, HNF4G, MSC, PRDM8, |
| transcription | | TARBP2, SNAI3, MEIS2, TSPYL2, MED27, RNF20, NR1H3 |
| GO:0030528-transcription regulator | 9 | SNAI3, MEIS2, GLIS1, MED27, ZNF24, HNF4G, MSC, |
| activity | | RNF20, NR1H3 |
| GO:0006355~regulation of | 10 | TARBP2, SNAI3, MEIS2, GLIS1, MED27, ZNF24, ZNF12, |
| transcription, DNA-dependent | | HNF4G, RNF20, NR1H3 |
| zinc finger region:C2H2-type 1 | 4 | SNAI3, GLIS1, ZNF24, ZNF12 |
| GO:0051252~regulation of RNA | 10 | TARBP2, SNAI3, MEIS2, GLIS1, MED27, ZNF24, ZNF12, |
| metabolic process | | HNF4G, RNF20, NR1H3 |
| GO:0003677~DNA binding | 12 | PRDM8, SNAI3, MEIS2, TSPYL2, GLIS1, ZNF24, H2AFY, |
| | | ZNF12, HNF4G, MSC, ZW10, NR1H3 |
| zinc finger region:C2H2-type 4 | 4 | SNAI3, GLIS1, ZNF24, ZNF12 |
| GO:0006350~transcription | 11 | PRDM8, ASXL3, SNAI3, TSPYL2, GLIS1, MED27, ZNF24, |
| | | ZNF12, HNF4G, MSC, NR1H3 |
| GO:0003700~transcription factor | 6 | SNAI3, MEIS2, ZNF24, HNF4G, MSC, NR1H3 |
| activity | | |
| zinc finger region:C2H2-type 2 | 4 | PRDM8, SNAI3, ZNF24, ZNF12 |
| IPR015880:Zinc finger, C2H2-like | 5 | PRDM8, SNAI3, GLIS1, ZNF24, ZNF12 |
| IPR013087:Zinc finger, C2H2- | 4 | SNAI3, GLIS1, ZNF24, ZNF12 |
| type/integrase, DNA-binding | | |
| SM00355:ZnF_C2H2 | 5 | PRDM8, SNAI3, GLIS1, ZNF24, ZNF12 |

Enrichment Score: 0.695896167722095

| | | |
|---|---|---|
| GO:0007610~behavior | 6 | CCL3, SLC1A3, CCL3L3, RAC1, ACSL4, MTNR1A |
| hsa04062:Chemokine signaling | 3 | CCL3, CCL3L3, RAC1 |
| pathway | | |
| cytokine | 3 | CCL3, CCL3L3, IL1A |
| GO:0006935~chemotaxis | 3 | CCL3, CCL3L3, RAC1 |
| GO:0042330~taxis | 3 | CCL3, CCL3L3, RAC1 |
| GO:0006954~inflammatory | 4 | CCL3, CCL3L3, RAC1, IL1A |
| response | | |
| GO:0009611~response to wounding | 5 | CCL3, SLC1A3, CCL3L3, RAC1, IL1A |
| GO:0005125~cytokine activity | 3 | CCL3, CCL3L3, IL1A |
| hsa04060:Cytokine-cytokine | 3 | CCL3, CCL3L3, IL1A |
| receptor interaction | | |
| GO:0006952~defense response | 5 | CCL3, IL27RA, CCL3L3, RAC1, IL1A |
| GO:0007626~locomotory behavior | 3 | CCL3, CCL3L3, RAC1 |
| GO:0005615~extracellular space | 4 | CCL3, CCL3L3, APOC1, IL1A |
| GO:0006955~immune response | 4 | CCL3, IL27RA, CCL3L3, IL1A |
| GO:0042592~homeostatic process | 3 | CCL3, RAC1, IL1A |

Enrichment Score: 0.6357971532121958

| | | |
|---|---|---|
| GO:0044421~extracellular region | 9 | CCL3, SLC1A3, HAPLN3, CRTAC1, CCL3L3, APOC1, |
| part | | MMP16, COL11A1, IL1A |
| GO:0005578~proteinaceous | 5 | SLC1A3, HAPLN3, CRTAC1, MMP16, COL11A1 |
| extracellular matrix | | |
| GO:0031012~extracellular matrix | 5 | SLC1A3, HAPLN3, CRTAC1, MMP16, COL11A1 |
| extracellular matrix | 4 | HAPLN3, CRTAC1, MMP16, COL11A1 |
| Secreted | 9 | CCL3, HAPLN3, CRTAC1, CCL3L3, APOC1, MMP16, |
| | | SERPINI1, COL11A1, IL1A |
| GO:0005576~extracellular region | 10 | CCL3, SLC1A3, HAPLN3, CRTAC1, CCL3L3, APOC1, |
| | | MMP16, SERPINI1, COL11A1, IL1A |
| signal | 12 | PCDHGA10, CCL3, HAPLN3, IL27RA, CRTAC1, CCL3L3, |
| | | APOC1, MMP16, SERPIN11, COL11A1, SDC2, DHCR24 |
| signal peptide | 12 | PCDHGA10, CCL3, HAPLN3, IL27RA, CRTAC1, CCL3L3, |
| | | APOC1, MMP16, SERPINI1, COL11A1, SDC2, DHCR24 |
| GO:0007186~G-protein coupled | 3 | CCL3, OR5K3, MTNR1A |
| receptor protein signaling pathway | | |
| disulfide bond | 7 | CCL3, HAPLN3, OR5K3, CRTAC1, CCL3L3, MMP16, |
| | | MTNR1A |
| disulfide bond | 7 | CCL3, HAPLN3, OR5K3, CRTAC1, CCL3L3, MMP16, |
| | | MTNR1A |

Enrichment Score: 0.5695523811903586

| | | |
|---|---|---|
| GO:0005730~nucleolus | 6 | MEIS2, TSPYL2, GLIS1, HNF4G, NOP10, RNF20 |
| GO:0051172~negative regulation of | 5 | MEIS2, TSPYL2, GLIS1, ZNF24, APOC1 |
| nitrogen compound metabolic | | |
| process | | |
| GO:0031327~negative regulation of | 5 | MEIS2, TSPYL2, GLIS1, ZNF24, APOC1 |
| cellular biosynthetic process | | |
| GO:0009890~negative regulation of | 5 | MEIS2, TSPYL2, GLIS1, ZNF24, APOC1 |
| biosynthetic process | | |
| GO:0010629~negative regulation of | 4 | TARBP2, MEIS2, GLIS1, ZNF24 |
| gene expression | | |
| GO:0045934~negative regulation of | 4 | MEIS2, TSPYL2, GLIS1, ZNF24 |
| nucleobase, nucleoside, nucleotide | | |
| and nucleic acid metabolic process | | |
| GO:0010605~negative regulation of | 5 | TARBP2, MEIS2, TSPYL2, GLIS1, ZNF24 |
| macromolecule metabolic process | | |
| GO:0010558~negative regulation of | 4 | MEIS2, TSPYL2, GLIS1, ZNF24 |
| macromolecule biosynthetic process | | |
| GO:0016481~negative regulation of | 3 | MEIS2, GLIS1, ZNF24 |
| transcription | | |

Enrichment Score: 0.5511302619302495

| | | |
|---|---|---|
| zinc finger region:RING-type | 3 | RAPSN, TRIM63, RNF20 |
| IPR018957:Zinc finger, C3HC4 | 3 | RAPSN, TRIM63, RNF20 |
| RING-type | | |
| IPR017907:Zinc finger, RING-type, | 3 | RAPSN, TRIM63, RNF20 |
| conserved site | | |
| IPR001841:Zinc finger, RING-type | 3 | RAPSN, TRIM63, RNF20 |
| SM00184:RING | 3 | RAPSN, TRIM63, RNF20 |

Enrichment Score: 0.5214545487219902

| | | |
|---|---|---|
| GO:0009628~response to abiotic | 5 | SLC1A3, DUSP1, TRPV2, RAC1, COL11A1 |
| stimulus | | |
| GO:0050890~cognition | 6 | CNNM4, SLC1A3, OR5K3, TRPV2, ACSL4, COL11A1 |
| GO:0050877~neurologica1 system | 7 | CNNM4, SLC1A3, OR5K3, RAPSN, TRPV2, ACSL4, |
| process | | COL11A1 |
| GO:0007600~sensory perception | 5 | CNNM4, SLC1A3, OR5K3, TRPV2, COL11A1 |
| GO:0006811~ion transport | 3 | CNNM4, SLC1A3, TRPV2 |

Enrichment Score: 0.5182365429181518

| | | |
|---|---|---|
| GO:0006366~transcription from | 3 | MED27, HNF4G, MSC |
| RNA polymerase II promoter | | |
| GO:0006351~transcription, DNA- | 3 | MED27, HNF4G, MSC |
| dependent | | |
| GO:0032774~RNA biosynthetic | 3 | MED27, HNF4G, MSC |
| process | | |

Enrichment Score: 0.5017372401227738

| | | |
|---|---|---|
| GO:0016337~cell-cell adhesion | 3 | PCDHGA10, CLDN6, COL11A1 |
| GO:0007155~cell adhesion | 5 | PCDHGA10, HAPLN3, CLDN6, RAC1, COL11A1 |
| GO:0022610~biological adhesion | 5 | PCDHGA10, HAPLN3, CLDN6, RAC1, COL11A1 |

Enrichment Score: 0.474737606246611

| | | |
|---|---|---|
| GO:0003712~transcription cofactor | 5 | MEIS2, MED27, MSC, RNF20, NR1H3 |
| activity | | |
| GO:0005730-nucleolus | 6 | MEIS2, TSPYL2, GLIS1, HNF4G, NOP10, RNF20 |
| GO:0008134~transcription factor | 5 | MEIS2, MED27, MSC, RNF20, NR1H3 |
| binding | | |
| GO:0006357~regulation of | 6 | TARBP2, MEIS2, GLIS1, MED27, HNF4G, NR1H3 |
| transcription from RNA polymerase | | |
| II promoter | | |
| GO:0003713~transcription | 3 | MED27, RNF20, NR1H3 |
| coactivator activity | | |
| GO:0016563~transcription activator | 4 | MED27, HNF4G, RNF20, NR1H3 |
| activity | | |
| GO:0045449~regulation of | 14 | ASXL3, GLIS1, ZNF24, ZNF12, HNF4G, MSC, PRDM8, |
| transcription | | TARBP2, SNAI3, MEIS2, TSPYL2, MED27, RNF20, NR1H3 |
| GO:0030528~transcription regulator | 9 | SNAI3, MEIS2, GLIS1, MED27, ZNF24, HNF4G, MSC, |
| activity | | RNF20, NR1H3 |
| GO:0010557~positive regulation of | 5 | MEIS2, GLIS1, RNF20, IL1A, NR1H3 |
| macromolecule biosynthetic process | | |
| GO:0006355~regulation of | 10 | TARBP2, SNAI3, MEIS2, GLIS1, MED27, ZNF24, ZNF12, |
| transcription, DNA-dependent | | HNF4G, RNF20, NR1H3 |
| GO:0031328~positive regulation of | 5 | MEIS2, GLIS1, RNF20, IL1A, NR1H3 |
| cellular biosynthetic process | | |
| GO:0009891~positive regulation of | 5 | MEIS2, GLIS1, RNF20, IL1A, NR1H3 |
| biosynthetic process | | |
| GO:0051252-regulation of RNA | 10 | TARBP2, SNAI3, MEIS2, GLIS1, MED27, ZNF24, ZNF12, |
| metabolic process | | HNF4G, RNF20, NR1H3 |
| GO:0045941~positive regulation of | 4 | MEIS2, GLIS1, RNF20, NR1H3 |
| transcription | | |
| GO:0010628~positive regulation of | 4 | MEIS2, GLIS1, RNF20, NR1H3 |
| gene expression | | |
| GO:0043228~non-membrane- | 12 | MEIS2, TSPYL2, RAPSN, GLIS1, H2AFY, HNF4G, TRIM63, |
| bounded organelle | | NOP10, KLHL3, RNF20, ZW10, FGD4 |
| GO:0043232~intracellular non- | 12 | MEIS2, TSPYL2, RAPSN, GLIS1, H2AFY, HNF4G, TRIM63, |
| membrane-bounded organelle | | NOP10, KLHL3, RNF20, ZW10, FGD4 |
| GO:0045944~positive regulation of | 3 | MEIS2, GLIS1, NR1H3 |
| transcription from RNA polymerase | | |
| II promoter | | |
| GO:0010604~positive regulation of | 5 | MEIS2, GLIS1, RNF20, IL1A, NR1H3 |
| macromolecule metabolic process | | |
| GO:0045935~positive regulation of | 4 | MEIS2, GLIS1, RNF20, NR1H3 |
| nucleobase, nucleoside, nucleotide | | |
| and nucleic acid metabolic process | | |
| GO:0051173~positive regulation of | 4 | MEIS2, GLIS1, RNF20, NR1H3 |
| nitrogen compound metabolic | | |
| process | | |
| GO:0031981~nuclear lumen | 7 | SNAI3, MEIS2, TSPYL2, GLIS1, HNF4G, NOP10, RNF20 |
| GO:0045893~positive regulation of | 3 | MEIS2, GLIS1, NR1H3 |
| transcription, DNA-dependent | | |
| GO:0051254~positive regulation of | 3 | MEIS2, GLIS1, NR1H3 |
| RNA metabolic process | | |
| GO:0070013~intracellular organelle | 7 | SNAI3, MEIS2, TSPYL2, GLIS1, HNF4G, NOP10, RNF20 |
| lumen | | |
| GO:0043233~organelle lumen | 7 | SNAI3, MEIS2, TSPYL2, GLIS1, HNF4G, NOP10, RNF20 |
| GO:0031974~membrane-enclosed | 7 | SNAI3, MEIS2, TSPYL2, GLIS1, HNF4G, NOP10, RNF20 |
| lumen | | |

Enrichment Score: 0.465581027382082

| | | |
|---|---|---|
| IPR001849:Pleckstrin homology | 3 | VEPH1, OSBPL9, FGD4 |
| IPR011993:Pleckstrin homology- | 3 | VEPH1, OSBPL9, FGD4 |
| type | | |
| SM00233:PH | 3 | VEPH1, OSBPL9, FGD4 |

Enrichment Score: 0.4066995149813719

| | | |
|---|---|---|
| GO:0007610~behavior | 6 | CCL3, SLC1A3, CCL3L3, RAC1, ACSL4, MTNR1A |
| GO:0000267~cell fraction | 4 | CCL3, SLC1A3, RAC1, ACSL4 |
| GO:0005624~membrane fraction | 3 | SLC1A3, RAC1, ACSL4 |
| GO:0005626~insoluble fraction | 3 | SLC1A3, RAC1, ACSL4 |

Enrichment Score: 0.3739835083948733

| | | |
|---|---|---|
| GO: 0016023~cytoplasmic | 4 | RAB3C, CAPN11, TRPV2, RAC1 |
| membrane-bounded vesicle | | |
| GO:0031988~membrane-bounded | 4 | RAB3C, CAPN11, TRPV2, RAC1 |
| vesicle | | |
| GO:0031410~cytoplasmic vesicle | 4 | RAB3C, CAPN11, TRPV2, RAC1 |
| GO:0031982~vesicle | 4 | RAB3C, CAPN11, TRPV2, RAC1 |

Enrichment Score: 0.20190763005512594

| | | |
|---|---|---|
| cell membrane | 12 | VEPH1, PCDHGA10, CNNM4, RAB3C, OR5K3, RAPSN, |
| | | CLDN6, TRPV2, RAC1, MMP16, RHOD, MTNR1A |
| GO:0044459~plasma membrane | 12 | VEPH1, SLC1A3, RAB3C, IL27RA, RAPSN, CLDN6, TRPV2, |
| part | | RAC1, MMP16, RHOD, SDC2, MTNR1A |
| GO:0005886~plasma membrane | 16 | PCDHGA10, CNNM4, RAB3C, IL27RA, OR5K3, CLDN6, |
| | | TRPV2, MMP16, SDC2, VEPH1, SLC1A3, RAPSN, RAC1, |
| | | RHOD, ACSL4, MTNR1A |
| topological domain:Extracellular | 10 | PCDHGA10, SLC1A3, IL27RA, OR5K3, CLDN6, TRPV2, |
| | | GGT2, MMP16, SDC2, MTNR1A |
| membrane | 22 | PCDHGA10, CNNM4, RAB3C, IL27RA, OR5K3, CLDN6, |
| | | TRPV2, ZDHHC7, GGT2, MMP16, SDC2, SVOPL, VEPH1, |
| | | SLC1A3, RAPSN, RAC1, FAM162A, RHOD, ACSL4, |
| | | MTNR1A, ZW10, DHCR24 |
| GO:0005887~integral to plasma | 5 | IL27RA, TRPV2, MMP16, SDC2, MTNR1A |
| membrane | | |
| glycoprotein | 15 | PCDHGA10, CNNM4, HAPLN3, IL27RA, OR5K3, CRTAC1, |
| | | TRPV2, GGT2, MMP16, SERPINI1, SDC2, SLC1A3, |
| | | COL11A1, IL1A, MTNR1A |
| GO:0031226~intrinsic to plasma | 5 | IL27RA, TRPV2, MMP16, SDC2, MTNR1A |
| membrane | | |
| topological domain:Cytoplasmic | 11 | PCDHGA10, SLC1A3, IL27RA, OR5K3, CLDN6, TRPV2, |
| | | GGT2, MMP16, ACSL4, SDC2, MTNR1A |
| transmembrane region | 16 | PCDHGA10, CNNM4, IL27RA, OR5K3, CLDN6, TRPV2, |
| | | ZDHHC7, GGT2, MMP16, SDC2, SVOPL, SLC1A3, |
| | | FAM162A, ACSL4, DHCR24, MTNR1A |
| transmembrane | 16 | PCDHGA10, CNNM4, IL27RA, OR5K3, CLDN6, TRPV2, |
| | | ZDHHC7, GGT2, MMP16, SDC2, SVOPL, SLC1A3, |
| | | FAM162A, ACSL4, DHCR24, MTNR1A |
| glycosylation site:N-linked | 11 | PCDHGA10, CNNM4, IL27RA, OR5K3, TRPV2, GGT2, |
| (GlcNAc . . . ) | | MMP16, SERPINI1, COL11A1, IL1A, MTNR1A |
| GO:0016021~integral to membrane | 16 | PCDHGA10, CNNM4, IL27RA, OR5K3, CLDN6, TRPV2, |
| | | ZDHHC7, GGT2, MMP16, SDC2, SVOPL, SLC1A3, |
| | | FAM162A, ACSL4, DHCR24, MTNR1A |
| GO:0031224~intrinsic to membrane | 16 | PCDHGA10, CNNM4, IL27RA, OR5K3, CLDN6, TRPV2, |
| | | ZDHHC7, GGT2, MMP16, SDC2, SVOPL, SLC1A3, |
| | | FAM162A, ACSL4, DHCR24, MTNR1A |

Enrichment Score: 0.1812991043816715

| | | |
|---|---|---|
| Protease | 3 | CAPN11, UCHL5, MMP16 |
| GO:0070011~peptidase activity, | 3 | CAPN11, UCHL5, MMP16 |
| acting on L-amino acid peptides | | |
| GO:0008233~peptidase activity | 3 | CAPN11, UCHL5, MMP16 |
| hydrolase | 5 | DUSP26, DUSP1, CAPN11, UCHL5, MMP16 |

Enrichment Score: 0.03908551349344777

| | | |
|---|---|---|
| GO:0017076~purine nucleotide | 7 | RAB3C, RAC1, ZNF12, RHOD, ACSL4, RRAGD, DHCR24 |
| binding | | |
| GO:0000166~nucleotide binding | 8 | RAB3C, APLF, RAC1, ZNF12, RHOD, ACSL4, RRAGD, |
| | | DHCR24 |
| GO:0030554~adenyl nucleotide | 3 | ZNF12, ACSL4, DHCR24 |
| binding | | |
| GO:0001883~purine nucleoside | 3 | ZNF12, ACSL4, DHCR24 |
| binding | | |
| GO:0001882~nucleoside binding | 3 | ZNF12, ACSL4, DHCR24 |

TABLE 5
Genes Negatively Correlated with TB

| | | |
|---|---|---|
| Term | Count | Genes |

Enrichment Score: 1.2865881937081116

| | | |
|---|---|---|
| golgi apparatus | 7 | PGAP3, DPY30, PNPLA8, ARHGEF2, SCYL1, SVIP, RTN3 |
| endoplasmic reticulum | 7 | PGAP3, PNPLA8, ELOVL3, UBE2V1P2, LMF1, SVIP, RTN3 |
| GO:0005794~Golgi apparatus | 7 | PGAP3, PNPLA8, ARHGEF2, SCYL1, SNX17, SVIP, RTN3 |
| GO:0005783~endoplasmic | 7 | PGAP3, PNPLA8, ELOVL3, UBE2V1P2, LMF1, SVIP, RTN3 |
| reticulum | | |

Enrichment Score: 1.193461933188953

| | | |
|---|---|---|
| cytoskeleton | 7 | ARHGEF2, SCYL1, DLGAP5, PPL, DMD, AKAP12, |
| | | CDC42SE1 |
| GO:0043228~non-membrane- | 14 | TUBBP5, ARHGEF2, SCYL1, DLGAP5, PPL, DMD, AKAP12, |
| bounded organelle | | CDC42SE1, TUBB8, MRPL45, CYB561, RPA4, TOP3B, RTN3 |
| GO:0043232~intracellular non- | 14 | TUBBP5, ARHGEF2, SCYL1, DLGAP5, PPL, DMD, AKAP12, |
| membrane-bounded organelle | | CDC42SE1, TUBB8, MRPL45, CYB561, RPA4, TOP3B, RTN3 |
| GO:0005856~cytoskeleton | 9 | TUBBP5, ARHGEF2, SCYL1, DLGAP5, PPL, DMD, AKAP12, |
| | | CDC42SE1, TUBB8 |

Enrichment Score: 1.1139583061715095

| | | |
|---|---|---|
| GO:0065003~macromolecular | 7 | SNAPC5, TUBBP5, BCS1L, TUBB8, GTF2B, NAP1L4, |
| complex assembly | | GCHFR |
| GO:0006461~protein complex | 6 | SNAPC5, TUBBP5, BCS1L, TUBB8, GTF2B, GCHFR |
| assembly | | |
| GO:0070271~protein complex | 6 | SNAPC5, TUBBP5, BCS1L, TUBB8, GTF2B, GCHFR |
| biogenesis | | |
| GO:0043933~macromolecular | 7 | SNAPC5, TUBBP5, BCS1L, TUBB8, GTF2B, NAP1L4, |
| complex subunit organization | | GCHFR |
| GO:0034622~cellular | 4 | TUBBP5, BCS1L, TUBB8, NAP1L4 |
| macromolecular complex assembly | | |
| GO:0043623~cellular protein | 3 | TUBBP5, BCS1L, TUBB8 |
| complex assembly | | |
| GO:0034621~cellular | 4 | TUBBP5, BCS1L, TUBB8, NAP1L4 |
| macromolecular complex subunit | | |
| organization | | |

Enrichment Score: 0.9686068589505084

| | | |
|---|---|---|
| GO:0006886~intracellular protein | 5 | ARHGEF2, SNX17, MGEA5, AKAP12, MRPL45 |
| transport | | |
| GO:0034613~cellular protein | 5 | ARHGEF2, SNX17, MGEA5, AKAP12, MRPL45 |
| localization | | |
| GO:0070727~cellular | 5 | ARHGEF2, SNX17, MGEA5, AKAP12, MRPL45 |
| macromolecule localization | | |
| GO:0046907~intracellular transport | 6 | ARHGEF2, SCYL1, SNX17, MGEA5, AKAP12, MRPL45 |
| GO:0008104~protein localization | 7 | ARHGEF2, SNX17, DMD, LMF1, MGEA5, AKAP12, MRPL45 |
| GO:0015031~protein transport | 6 | ARHGEF2, SNX17, LMF1, MGEA5, AKAP12, MRPL45 |
| GO:0045184~establishment of | 6 | ARHGEF2, SNX17, LMF1, MGEA5, AKAP12, MRPL45 |
| protein localization | | |

Enrichment Score: 0.7602205187662866

| | | |
|---|---|---|
| GO:0005856~cytoskeleton | 9 | TUBBP5, ARHGEF2, SCYL1, DLGAP5, PPL, DMD, AKAP12, |
| | | CDC42SE1, TUBB8 |
| GO:0015630~microtubule | 5 | TUBBP5, ARHGEF2, SCYL1, DLGAP5, TUBB8 |
| cytoskeleton | | |
| GO:0005874~microtubule | 3 | TUBBP5, ARHGEF2, TUBB8 |
| GO:0044430~cytoskeletal part | 5 | TUBBP5, ARHGEF2, SCYL1, DLGAP5, TUBB8 |

Enrichment Score: 0.6295977322358206

| | | |
|---|---|---|
| GO:0005525~GTP binding | 5 | TUBBP5, RABL3, RAB28, TUBB8, PCK2 |
| GO:0032561~guanyl ribonucleotide | 5 | TUBBP5, RABL3, RAB28, TUBB8, PCK2 |
| binding | | |
| GO:0019001~guanyl nucleotide | 5 | TUBBP5, RABL3, RAB28, TUBB8, PCK2 |
| binding | | |
| nucleotide phosphate-binding | 4 | RABL3, RAB28, TUBB8, PCK2 |
| region: GTP | | |
| gtp-binding | 4 | RABL3, RAB28, TUBB8, PCK2 |
| GO:0032553~ribonucleotide | 11 | TUBBP5, PNPLA8, RABL3, SCYL1, RAB28, HSPA14, |
| binding | | BCS1L, TUBB8, PCK2, CDKL2, TOP3B |
| GO:0032555~purine ribonucleotide | 11 | TUBBP5, PNPLA8, RABL3, SCYL1, RAB28, HSPA14, |
| binding | | BCS1L, TUBB8, PCK2, CDKL2, TOP3B |
| GO:0017076~purine nucleotide | 11 | TUBBP5, PNPLA8, RABL3, SCYL1, RAB28, HSPA14, |
| binding | | BCS1L, TUBB8, PCK2, CDKL2, TOP3B |
| GO:0003924~GTPase activity | 3 | TUBBP5, RAB28, TUBB8 |
| GO:0000166~nucleotide binding | 12 | HNRNPL, TUBBP5, PNPLA8, RABL3, SCYL1, RAB28, |
| | | HSPA14, BCS1L, TUBB8, PCK2, CDKL2, TOP3B |
| nucleotide-binding | 8 | RABL3, RAB28, HSPA14, BCS1L, TUBB8, PCK2, CDKL2, |
| | | TOP3B |
| GO:0005524~ATP binding | 6 | PNPLA8, SCYL1, HSPA14, BCS1L, CDKL2, TOP3B |
| GO:0032559~adenyl ribonucleotide | 6 | PNPLA8, SCYL1, HSPA14, BCS1L, CDKL2, TOP3B |
| binding | | |
| GO:0030554~adenyl nucleotide | 6 | PNPLA8, SCYL1, HSPA14, BCS1L, CDKL2, TOP3B |
| binding | | |
| GO:0001883~purine nucleoside | 6 | PNPLA8, SCYL1, HSPA14, BCS1L, CDKL2, TOP3B |
| binding | | |
| GO:0001882~nucleoside binding | 6 | PNPLA8, SCYL1, HSPA14, BCS1L, CDKL2, TOP3B |
| atp-binding | 4 | HSPA14, BCS1L, CDKL2, TOP3B |

Enrichment Score: 0.4766169112180347

| | | |
|---|---|---|
| GO:0031090~organelle membrane | 7 | IMMP1L, PGAP3, PNPLA8, SCYL1, BCS1L, MRPL45, |
| | | GCHFR |
| GO:0005743~mitochondrial inner | 3 | IMMP1L, BCS1L, MRPL45 |
| membrane | | |
| GO:0005739~mitochondrion | 6 | IMMP1L, SIVA1, PPL, BCS1L, PCK2, MRPL45 |
| GO:0019866-organelle inner | 3 | IMMP1L, BCS1L, MRPL45 |
| membrane | | |
| GO:0031967~organelle envelope | 4 | IMMP1L, BCS1L, MRPL45, GCHFR |
| GO:0031975~envelope | 4 | IMMP1L, BCS1L, MRPL45, GCHFR |
| mitochondrion | 5 | IMMP1L, PPL, BCS1L, PCK2, MRPL45 |
| GO:0031966~mitochondrial | 3 | IMMP1L, BCS1L, MRPL45 |
| membrane | | |
| GO:0005740~mitochondrial | 3 | IMMP1L, BCS1L, MRPL45 |
| envelope | | |
| GO:0044429~mitochondrial part | 3 | IMMP1L, BCS1L, MRPL45 |

Enrichment Score: 0.4269912862695477

| | | |
|---|---|---|
| GO:0008219~cell death | 5 | SIVA1, ARHGEF2, PDCD10, MGEA5, RTN3 |
| GO:0016265~death | 5 | SIVA1, ARHGEF2, PDCD10, MGEA5, RTN3 |
| Apoptosis | 3 | SIVA1, PDCD10, RTN3 |
| GO:0006915~apoptosis | 4 | SIVA1, ARHGEF2, PDCD10, RTN3 |
| GO: 0012501~programmed cell | 4 | SIVA1, ARHGEF2, PDCD10, RTN3 |
| death | | |

Enrichment Score: 0.3835607614553488

| | | |
|---|---|---|
| GO:0043065~positive regulation of | 4 | SIVA1, ARHGEF2, SSTR3, RARB |
| apoptosis | | |
| GO:0043068~positive regulation of | 4 | SIVA1, ARHGEF2, SSTR3, RARB |
| programmed cell death | | |
| GO:0010942~positive regulation of | 4 | SIVA1, ARHGEF2, SSTR3, RARB |
| cell death | | |
| GO:0006917~induction of apoptosis | 3 | SIVA1, ARHGEF2, SSTR3 |
| GO:0012502~induction of | 3 | SIVA1, ARHGEF2, SSTR3 |
| programmed cell death | | |
| GO:0042981~regulation of | 4 | SIVA1, ARHGEF2, SSTR3, RARB |
| apoptosis | | |
| GO:0043067~regulation of | 4 | SIVA1, ARHGEF2, SSTR3, RARB |
| programmed cell death | | |
| GO:0010941~regulation of cell | 4 | SIVA1, ARHGEF2, SSTR3, RARB |
| death | | |
| GO:0042127~regulation of cell | 3 | ARHGEF2, SSTR3, RARB |
| proliferation | | |

Enrichment Score: 0.2971377682178453

| | | |
|---|---|---|
| GO:0046914~transition metal ion | 14 | SIVA1, ZNF831, ARHGEF2, ZNF92, PCK2, RNF181, CYB561, |
| binding | | GTF2B, FAM90A24P, APOBEC3A, DMD, ZNF426, RARB, CP |
| GO:0008270~zinc ion binding | 11 | SIVA1, ZNF831, APOBEC3A, ARHGEF2, ZNF92, DMD, |
| | | ZNF426, RARB, RNF181, GTF2B, FAM90A24P |
| metal-binding | 13 | SIVA1, ZNF831, ARHGEF2, ZNF92, PCK2, RNF181, CYB561, |
| | | GTF2B, APOBEC3A, DMD, ZNF426, RARB, CP |
| zinc | 10 | SIVA1, ZNF831, APOBEC3A, ARHGEF2, ZNF92, DMD, |
| | | ZNF426, RARB, RNF181, GTF2B |
| zinc-finger | 8 | ZNF831, ARHGEF2, ZNF92, DMD, ZNF426, RARB, RNF181, |
| | | GTF2B |
| GO:0046872~metal ion binding | 14 | SIVA1, ZNF831, ARHGEF2, ZNF92, PCK2, RNF181, CYB561, |
| | | GTF2B, FAM90A24P, APOBEC3A, DMD, ZNF426, RARB, CP |
| GO:0043169~cation binding | 14 | SIVA1, ZNF831, ARHGEF2, ZNF92, PCK2, RNF181, CYB561, |
| | | GTF2B, FAM90A24P, APOBEC3A, DMD, ZNF426, RARB, CP |
| GO:0043167~ion binding | 14 | SIVA1, ZNF831, ARHGEF2, ZNF92, PCK2, RNF181, CYB561, |
| | | GTF2B, FAM90A24P, APOBEC3A, DMD, ZNF426, RARB, CP |

Enrichment Score: 0.2186893313759546

| | | |
|---|---|---|
| G protein-coupled receptor | 3 | MCHR2, SSTR3, OPN1LW |
| PIRSF800006:rhodopsin-like G | 4 | MCHR2, OR51I2, SSTR3, OPN1LW |
| protein-coupled receptors | | |
| GO:0007186~G-protein coupled | 6 | MCHR2, OR51I2, SSTR3, OR2B3, OPN1LW, AKAP12 |
| receptor protein signaling pathway | | |
| IPR017452:GPCR, rhodopsin-like | 4 | MCHR2, OR51I2, SSTR3, OPN1LW |
| superfamily | | |
| IPR000276:7TM GPCR, rhodopsin- | 4 | MCHR2, OR51I2, SSTR3, OPN1LW |
| like | | |
| GO:0050877~neurologica1 system | 6 | OR51I2, 0R2B3, OPN1LW, DMD, IL1RAPL1, GCHFR |
| process | | |
| receptor | 7 | MCHR2, OR51I2, SSTR3, OPN1LW, PLXNB2, RARB, |
| | | IL1RAPL1 |
| g-protein coupled receptor | 4 | MCHR2, OR51I2, SSTR3, OPN1LW |
| GO:0031224~intrinsic to membrane | 19 | PGAP3, MCHR2, OR2B3, OPN1LW, PLXNB2, LMF1, |
| | | CDC42SE1, BCS1L, CYB561, RTN3, PNPLA8, SSTR3, |
| | | OR51I2, 5LC26A7, ELOVL3, UBE2V1P2, DNAJC4, CP, |
| | | IL1RAPL1 |
| transducer | 4 | MCHR2, OR51I2, SSTR3, OPN1LW |
| membrane | 23 | PGAP3, MCHR2, OPN1LW, PLXNB2, LMF1, CDC42SE1, |
| | | BCS1L, CYB561, RTN3, IMMP1L, PNPLA8, SSTR3, OR51I2, |
| | | RAB28, SLC26A7, PPL, ELOVL3, DMD, UBE2V1P2, SVIP, |
| | | DNAJC4, IL1RAPL1, GCHFR |
| transmembrane protein | 3 | MCHR2, SSTR3, OPN1LW |
| GO:0050890~cognition | 4 | OR51I2, 0R2B3, OPN1LW, IL1RAPL1 |
| GO:0016021~integral to membrane | 17 | PGAP3, MCHR2, OR2B3, OPN1LW, PLXNB2, LMF1, BCS1L, |
| | | CYB561, RTN3, PNPLA8, SSTR3, OR51I2, SLC26A7, |
| | | ELOVL3, UBE2V1P2, DNAJC4, IL1RAPL1 |
| GO:0007600~sensory perception | 3 | OR5112, OR2B3, OPN1LW |
| transmembrane region | 16 | PGAP3, MCHR2, OPN1LW, PLXNB2, LMF1, BCS1L, |
| | | CYB561, RTN3, PNPLA8, SSTR3, OR51I2, SLC26A7, |
| | | ELOVL3, UBE2V1P2, DNAJC4, IL1RAPL1 |
| transmembrane | 16 | PGAP3, MCHR2, OPN1LW, PLXNB2, LMF1, BCS1L, |
| | | CYB561, RTN3, PNPLA8, SSTR3, OR51I2, 5LC26A7, |
| | | ELOVL3, UBE2V1P2, DNAJC4, IL1RAPL1 |
| GO:0007166~cell surface receptor | 6 | MCHR2, OR5112, SSTR3, OR2B3, OPN1LW, AKAP12 |
| linked signal tmnsduction | | |
| topological domain:Cytoplasmic | 9 | PGAP3, MCHR2, OR51I2, SSTR3, OPN1LW, SLC26A7, |
| | | PLXNB2, CYB561, IL1RAPL1 |
| topological domain:Extracellular | 7 | MCHR2, OR51I2, SSTR3, OPN1LW, SLC26A7, PLXNB2, |
| | | IL1RAPL1 |
| glycosylation site:N-linked | 9 | PGAP3, PNPLA8, MCHR2, OR51I2, SSTR3, OPN1LW, |
| (GlcNAc . . . ) | | PLXNB2, CP, IL1RAPL1 |
| glycoprotein | 9 | PGAP3, PNPLA8, MCHR2, OR51I2, SSTR3, OPN1LW, |
| | | PLXNB2, CP, IL1RAPL1 |

Enrichment Score: 0.1710474073325014

| | | |
|---|---|---|
| zinc-finger | 8 | ZNF831, ARHGEF2, ZNF92, DMD, ZNF426, RARB, RNF181, |
| | | GTF2B |
| IPR013087:Zinc finger, C2H2- | 3 | ZNF831, ZNF92, ZNF426 |
| type/integrase, DNA-binding | | |
| SM00355:ZnF_C2H2 | 3 | ZNF831, ZNF92, ZNF426 |
| IPR007087:Zinc finger, C2H2-type | 3 | ZNF831, ZNF92, ZNF426 |
| IPR015880:Zinc finger, C2H2-like | 3 | ZNF831, ZNF92, ZNF426 |

Enrichment Score: 0.16076605592743434

| | | |
|---|---|---|
| SM00349:KRAB | 3 | ZNF92, SSX6, ZNF426 |
| IPR001909:Krueppel-associated | 3 | ZNF92, SSX6, ZNF426 |
| box | | |
| transcription regulation | 8 | SNAPC5, DPY30, SCYL1, ZNF92, SSX6, ZNF426, RARB, |
| | | GTF2B |
| Transcription | 8 | SNAPC5, DPY30, SCYL1, ZNF92, SSX6, ZNF426, RARB, |
| | | GTF2B |
| GO:0045449~regulation of | 10 | SIVA1, SNAPC5, SCYL1, ZNF92, DMD, UBE2V1P2, SSX6, |
| transcription | | ZNF426, RARB, GTF2B |
| GO:0003677~DNA binding | 8 | SNAPC5, SCYL1, ZNF92, ZNF426, RARB, GTF2B, RPA4, |
| | | TOP3B |
| GO:0030528~transcription regulator | 5 | SNAPC5, ZNF92, UBE2V1P2, RARB, GTF2B |
| activity | | |
| GO:0006355~regulation of | 6 | ZNF92, UBE2V1P2, SSX6, ZNF426, RARB, GTF2B |
| transcription, DNA-dependent | | |
| GO:0006350~transcription | 7 | SNAPC5, SCYL1, ZNF92, SSX6, ZNF426, RARB, GTF2B |
| GO:0051252~regulation of RNA | 6 | ZNF92, UBE2V1P2, SSX6, ZNF426, RARB, GTF2B |
| metabolic process | | |
| GO:0003700~transcription factor | 3 | SNAPC5, ZNF92, RARB |
| activity | | |
| dna-binding | 5 | SCYL1, ZNF92, ZNF426, RARB, TOP3B |

Enrichment Score: 0.1572294185208537

| | | |
|---|---|---|
| host-virus interaction | 3 | SIVA1, GTF2B, RTN3 |
| GO:0031981~nuclear lumen | 5 | HNRNPL, SIVA1, GTF2B, CYB561, RTN3 |
| GO:0005654~nucleoplasm | 3 | HNRNPL, SIVA1, GTF2B |
| GO:0070013~intracellular organelle | 5 | HNRNPL, SIVA1, GTF2B, CYB561, RTN3 |
| lumen | | |
| GO:0043233~organelle lumen | 5 | HNRNPL, SIVA1, GTF2B, CYB561, RTN3 |
| GO:0031974~membrane-enclosed | 5 | HNRNPL, SIVA1, GTF2B, CYB561, RTN3 |
| lumen | | |

Enrichment Score: 0.12193299079639833

| | | |
|---|---|---|
| GO:0000267~cell fraction | 4 | PNPLA8, DMD, DNAJC4, GCHFR |
| GO:0005624~membrane fraction | 3 | PNPLA8, DMD, DNAJC4 |
| GO:0005626~insoluble fmction | 3 | PNPLA8, DMD, DNAJC4 |

Enrichment Score: 0.031540863257107116

| | | |
|---|---|---|
| disulfide bond | 9 | AADACL2, OR51I2, RNASE11, SSTR3, OPN1LW, MGEA5, |
| | | CP, IGKC, IL1RAPL1 |
| disulfide bond | 9 | AADACL2, OR51I2, RNASE11, SSTR3, OPN1LW, MGEA5, |
| | | CP, IGKC, IL1RAPL1 |
| GO:0005576~extracellular region | 5 | AADACL2, RNASE11, CP, IGKC, RTN3 |
| signal | 7 | AADACL2, PGAP3, RNASE11, PLXNB2, CP, IGKC, |
| | | IL1RAPL1 |
| signal peptide | 7 | AADACL2, PGAP3, RNASE11, PLXNB2, CP, IGKC, |
| | | IL1RAPL1 |
| Secreted | 3 | AADACL2, RNASE11, CP |

### Example 3. A Comprehensive Single Cell Atlas of Non-Human Primate Cell During Homeostasis and Pathogenic Infection

Immune systems play an essential role in ensuring our health. From decades of laboratory and clinical work, there has been a basic understanding of immune balance and its importance for a healthy immune system. For example, hyperactivity can lead to allergy, inflammation, tissue damage, autoimmune disease and excessive cellular death. On the other hand, immunodeficiency can lead to outgrowth of cancers and the inability to kill or suppress external invaders. The immune system has evolved multiple modalities and redundancies that balance the system, including but not limited to memory, exhaustion, anergy, and senescence.

As the gene-expression program of a given cell closely reflects both its identity and function (Heinz et al., 2015), a systematic atlas of single-cell RNA profiles can help address many questions about immune regulations, their networks and molecular processes, and the response to pathogenic stimuli. Given the importance of the immune system, a systematic understanding of immune regulations on cell, tissue, and organism levels is crucial for clinicians and researchers to efficiently diagnose and develop treatments for immune system related disease.

Here, using scRNA-seq, this study identified gene signatures involved in SHIV-infection and immune responses, characterized cellular heterogeneity within specific cell-types, and demonstrated how these cell types and states change dynamically at different states of infection. More importantly, this study provides a resourceful pan-tissue database of expression profiles of healthy non-human primate that serves as a detailed reference data set for follow up studies regarding HIV as well as more disease and pathogenic states. Given the resemblance between HIV and SHIV, and the kinship between human and non-human primates, the atlas disclosed by this study also allows for parallel comparison and identifications of specific sub cell types as well as differentially regulated genes involved in human HIV infection.

Four Rhesus Macaques were sacrificed with full necropsy. Single cells from 12 distinct tissues were collected and single cell RNA-Sequencing was performed on these cells. Three Rhesus Macaques were infected with SHIV for 6 months, initiated anti-retroviral therapy for 6 months, and then sacrificed with full necropsy. Eight distinct tissue per SHIV+ animal was collected for single cell RNA-Sequencing. Tissues were collected as population controls in multiple forms, including RNALater, paraffin embedded, live cells frozen, lysed post dissociation and saved for control experiments and validations.

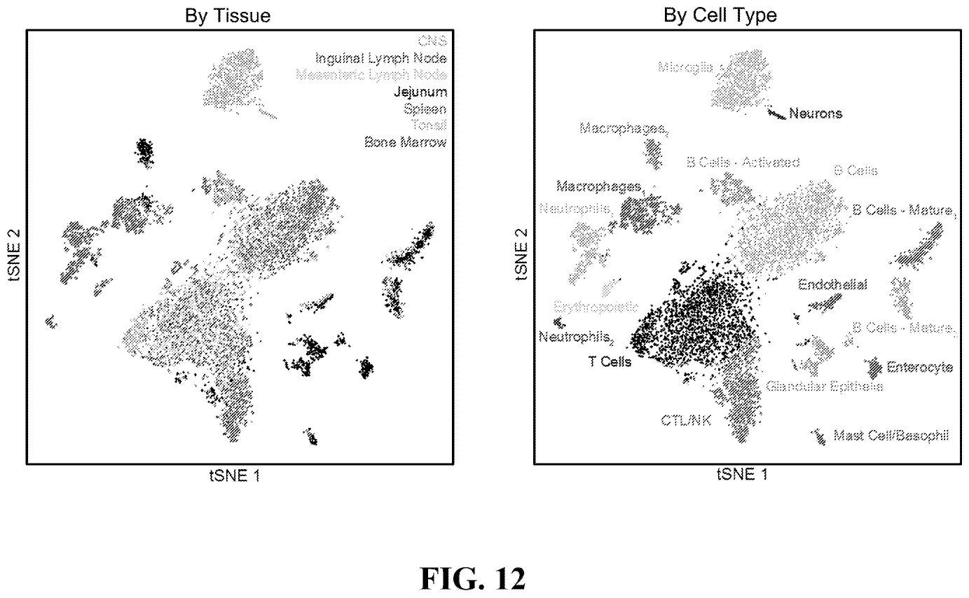

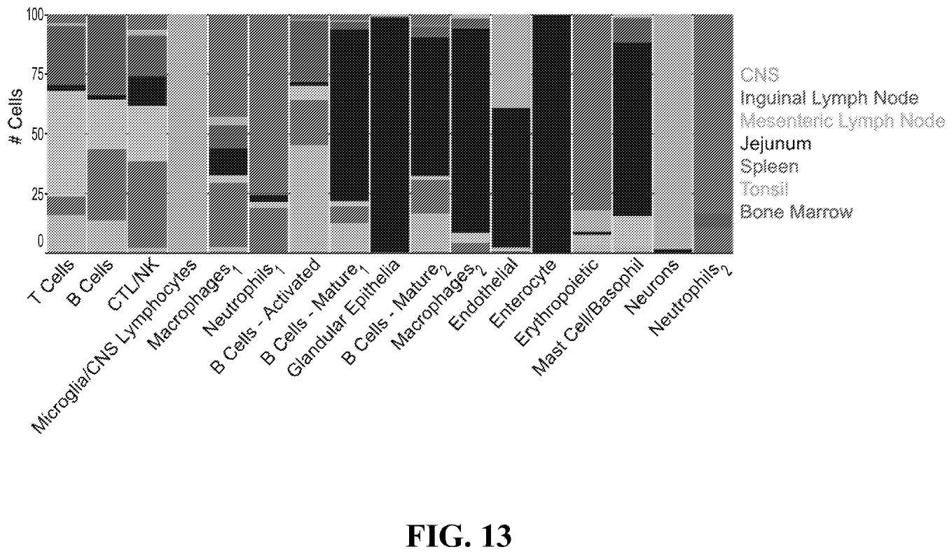

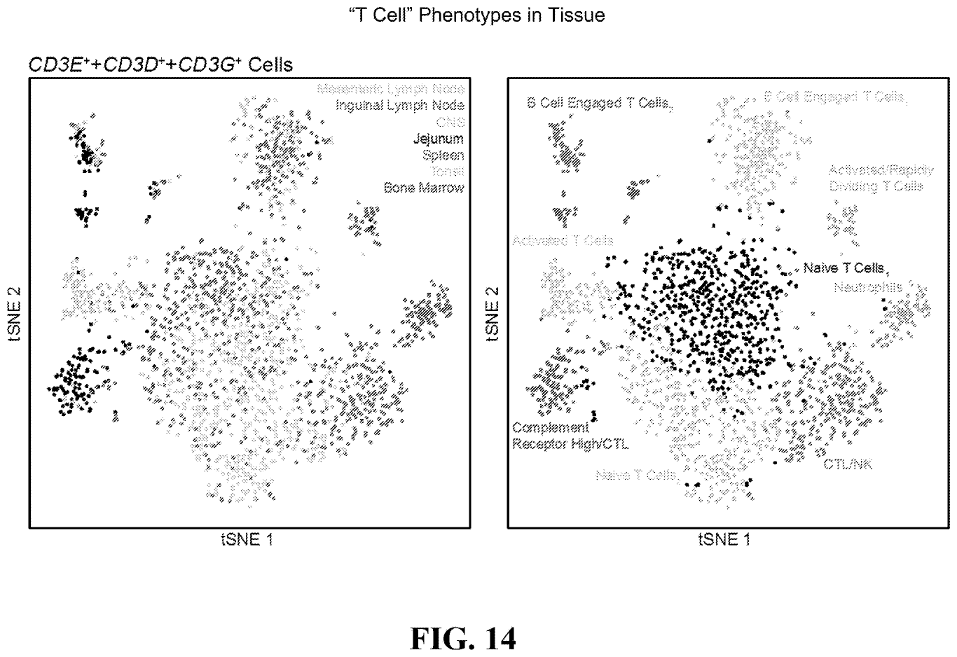

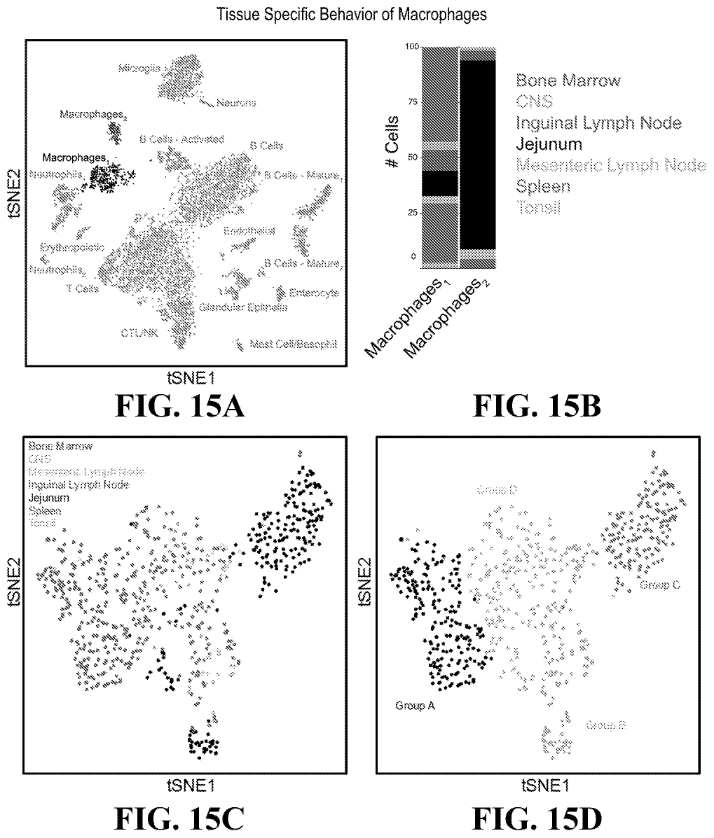

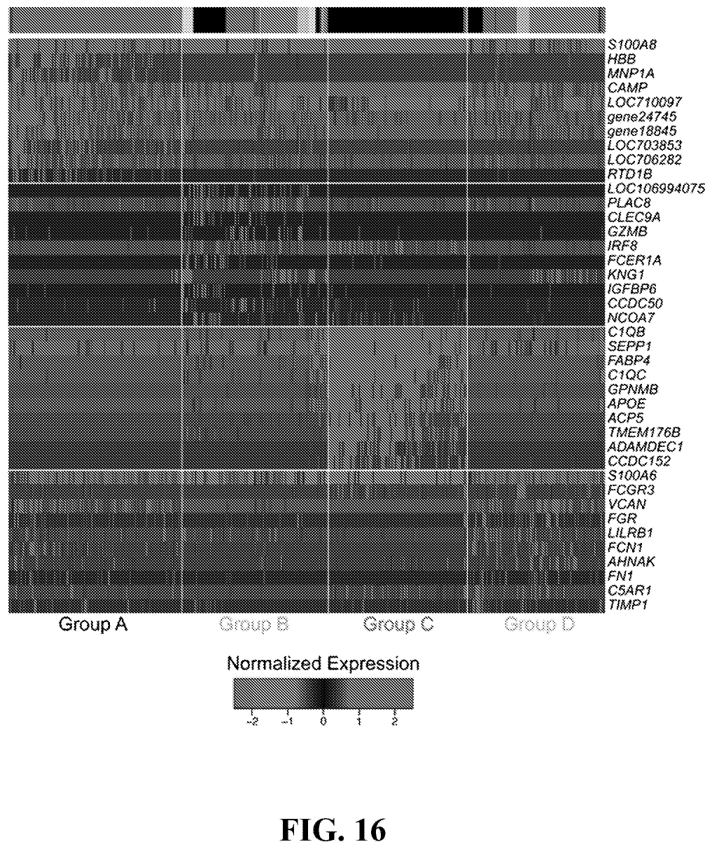

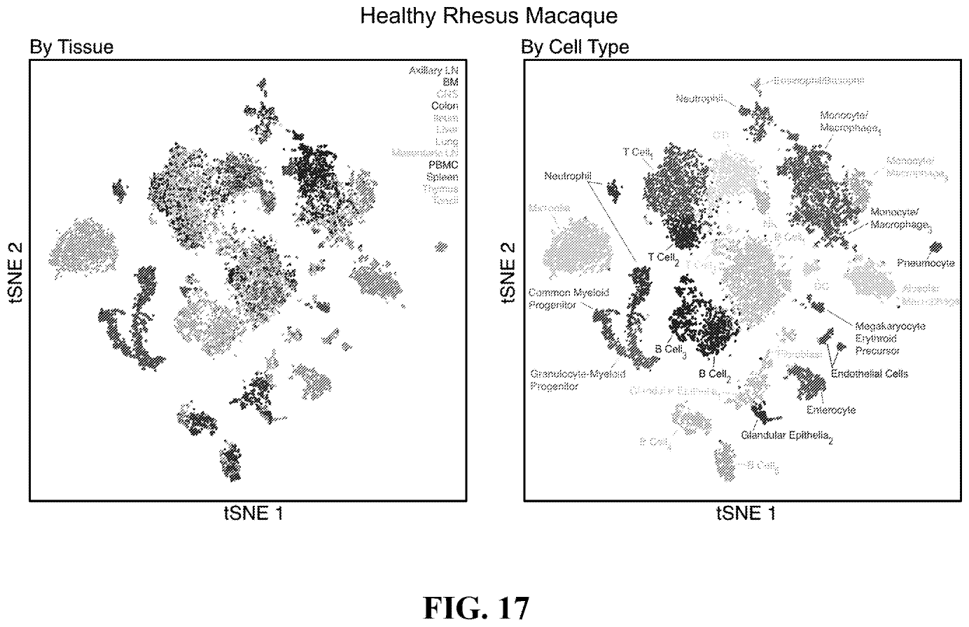

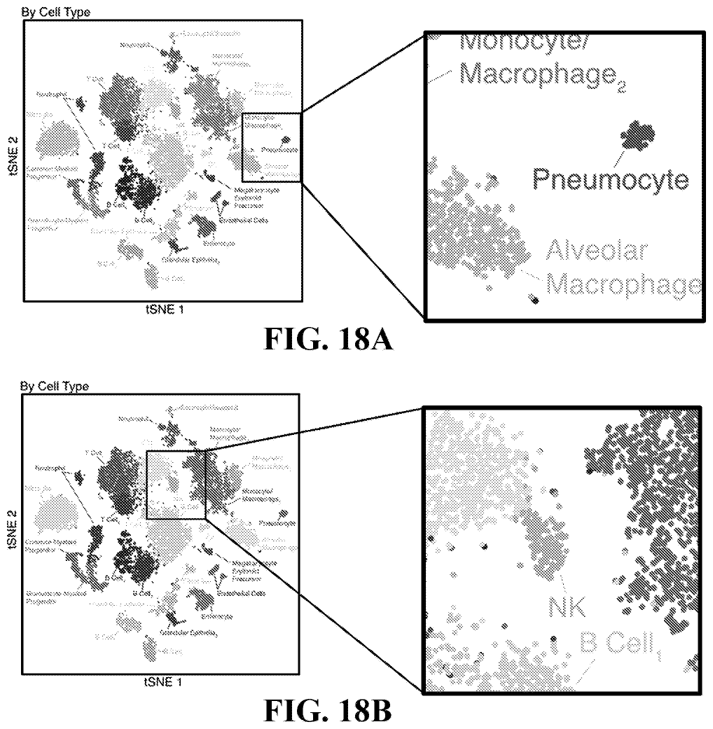

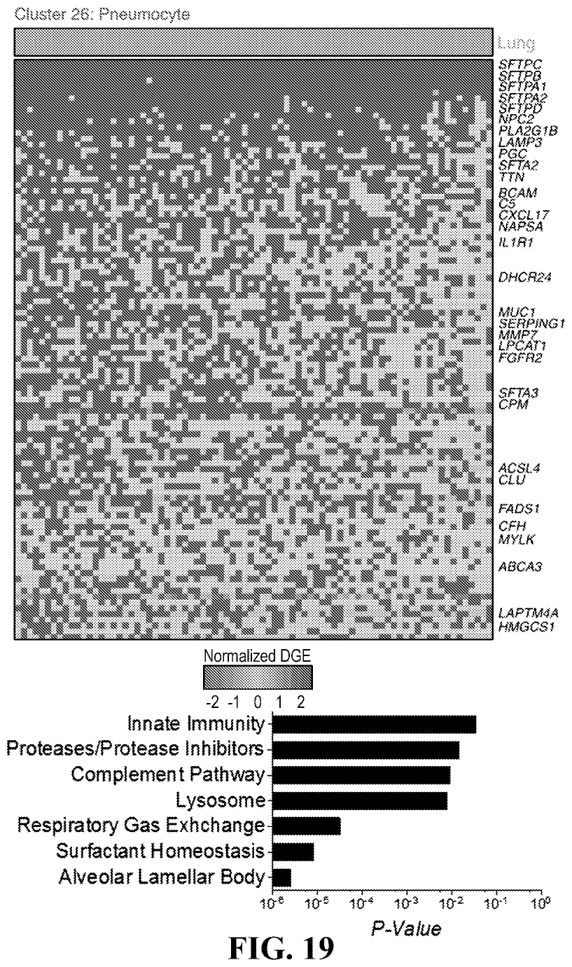

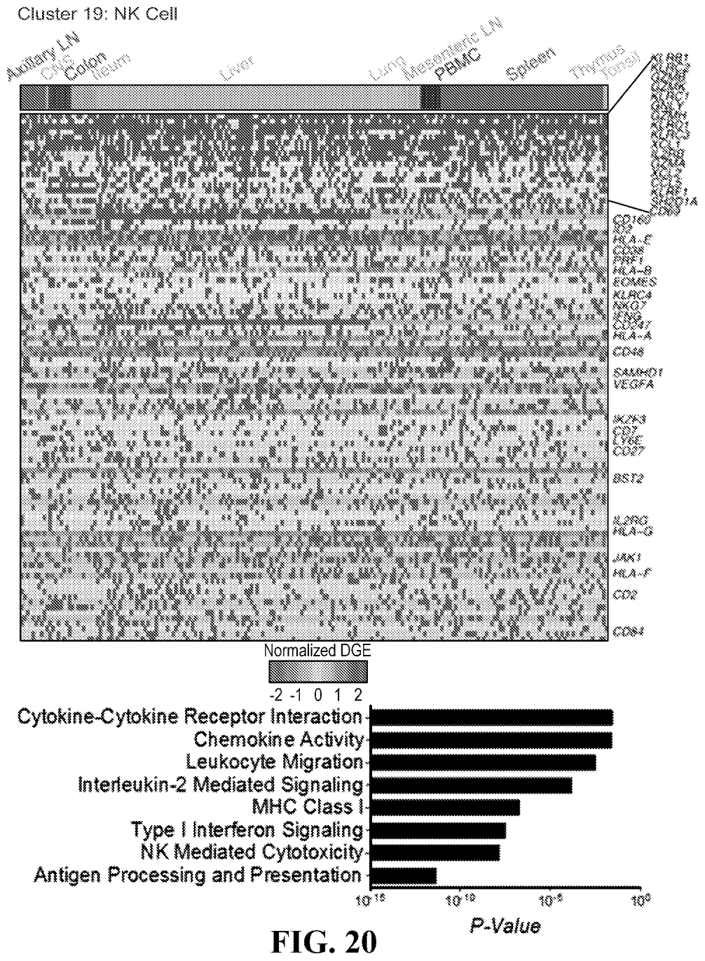

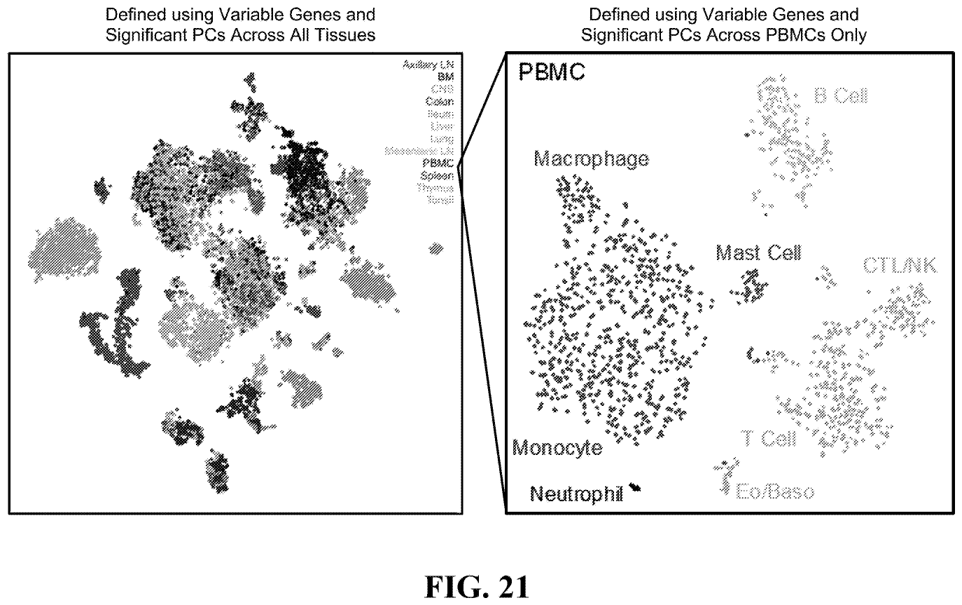

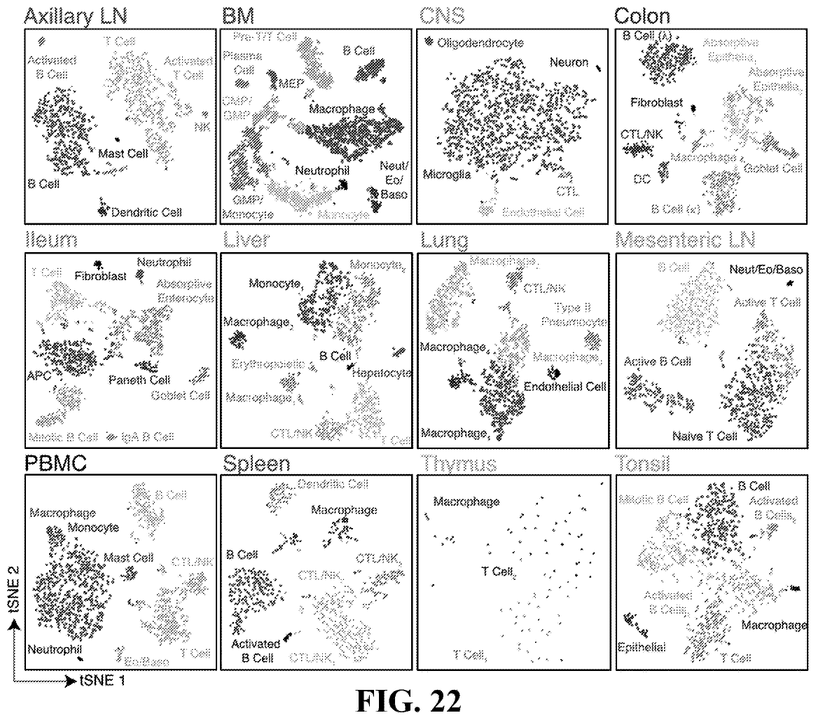

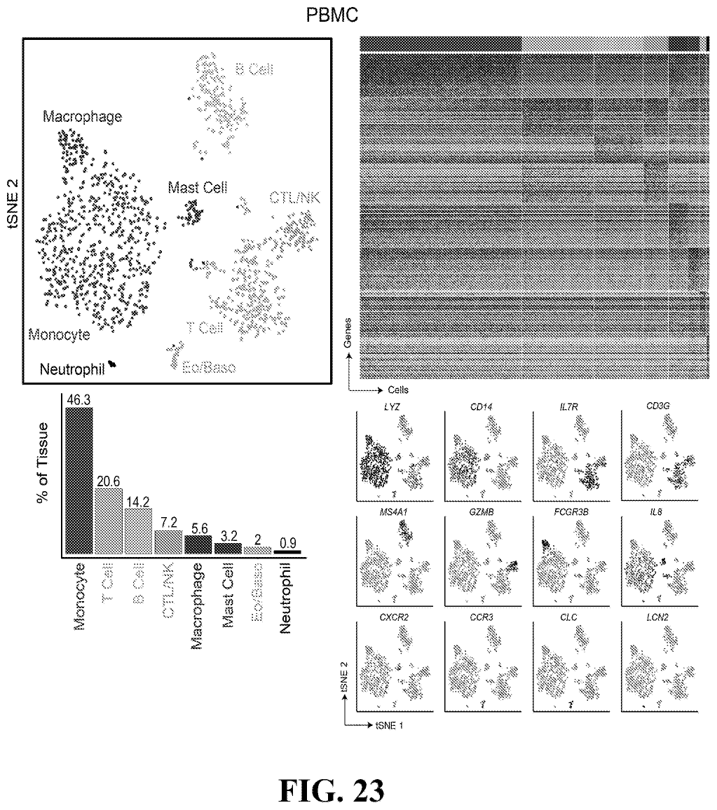

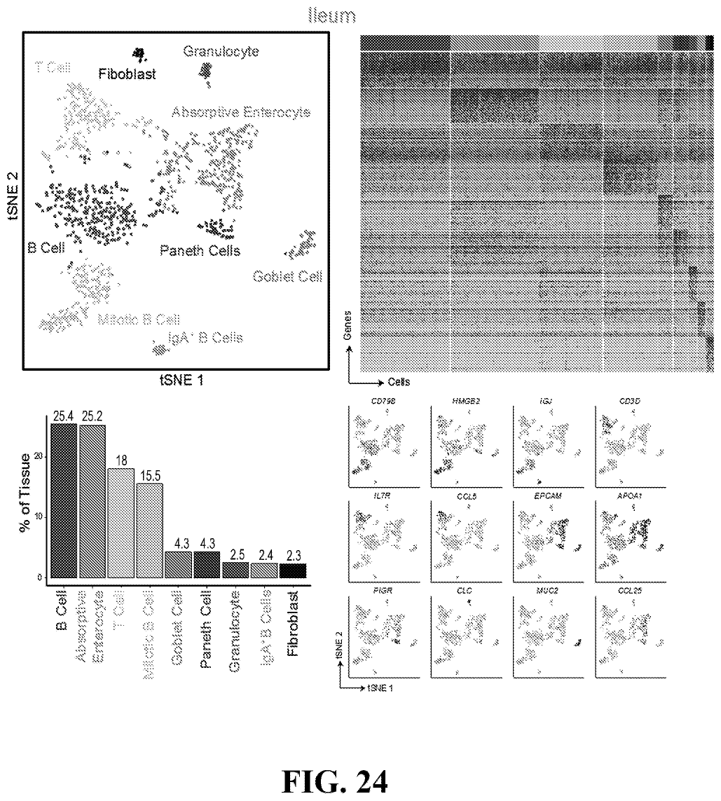

Single cell sequencing data was partitioned and annotated with supervised clustering, the results of which were visualized using tSNE (Amir el et al., 2013; Shekhar et al., 2016; van der Maaten and Hinton, 2008a). Based on expression profiles, individual cells are clustered and defined by tissues and cell types (FIG. 12-13). Particularly, this study identifies tissue specific phenotypes and behaviors of T cells (CD3E+, CD3D+, and CD3G+ cells), neutrophils, microglia, B cells, glandular epithelia, enterocytes, fibroblasts, megakaryocytes, erythroid precursor, DC, NK, macrophages, pneumocytes, eosinophil, and basophil cells are differentiated by expression profiles in axillary lymph nodes, central nerve system, colon, ileum, liver, lung, mesenteric lymph nodes, blood, spleen, thymus, and tonsil tissues, as illustrated in FIGS. 14-24. Specifically, in macrophages from different tissues, gene expression (S100A8, HBB, MNP1A, CAMP, LOC710097, gene 24745, gene 18845, LOC703853, LOC706282, RTD1B, LOC106994075, PLAC8, CLEC9A, GZMB, IRF8, FCER1A, KNG1, IGFBP6, CCDC50, NCOA7, C1QB, SEPP1, FABP4, C1QC, GPNMB, APOE, ACP5, YMRM176B, ADAMDEC1, CCDC 152, S100A6, FCGR3, VCAN, FGR, LILRB1, FCN1, AHNAK, FN1, C5AR1, TIMP1) distinguishes individual cells by their tissue of origins (FIG. 16).

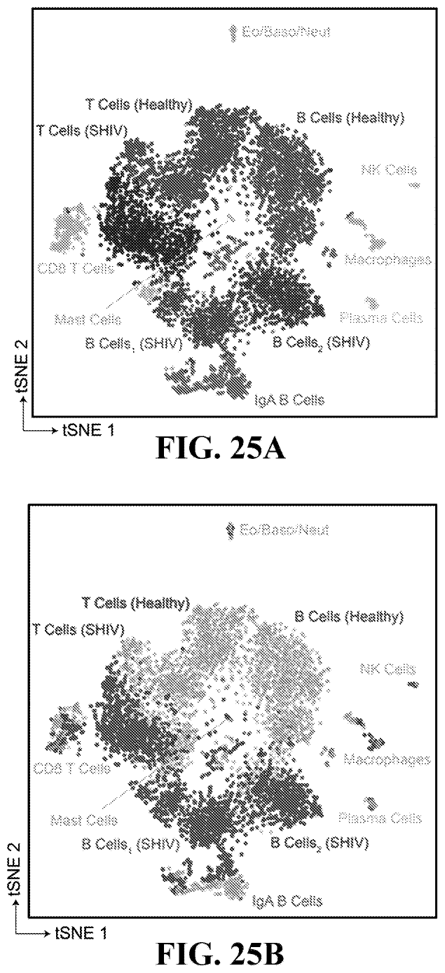

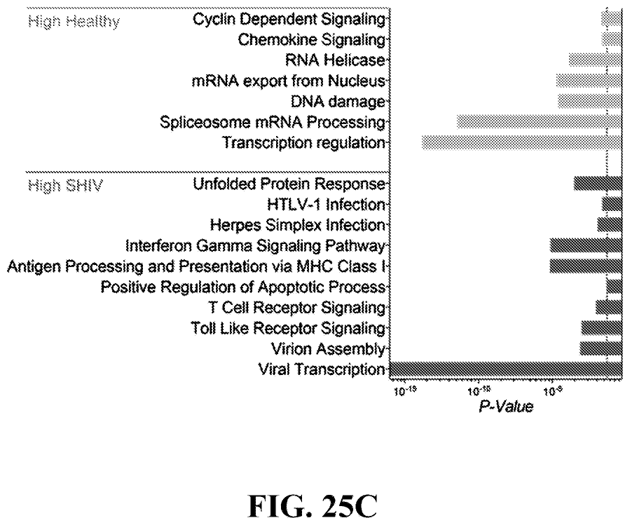

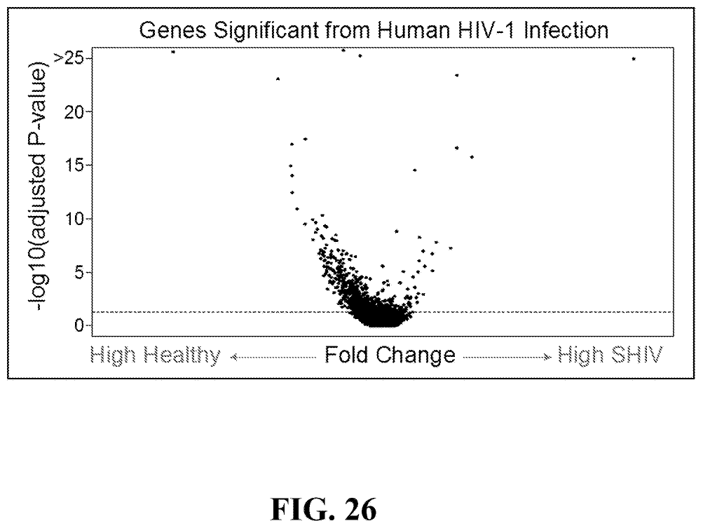

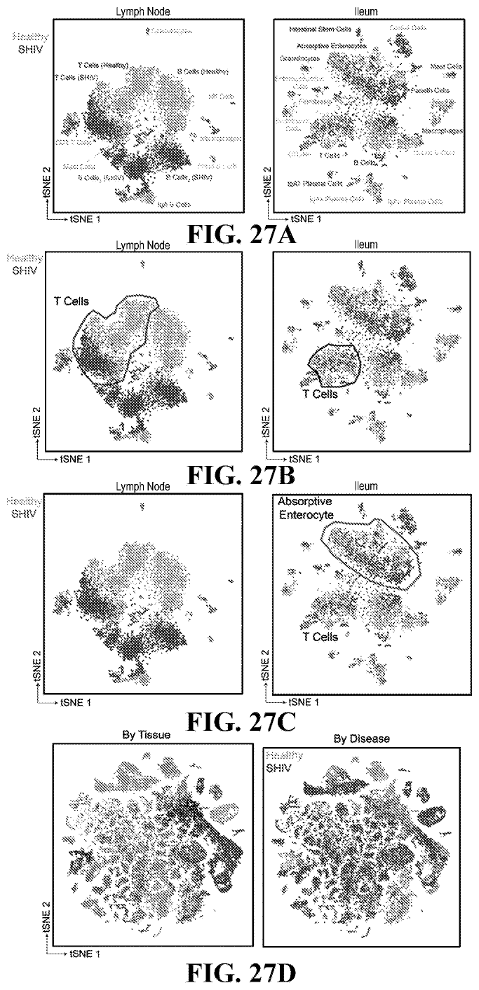

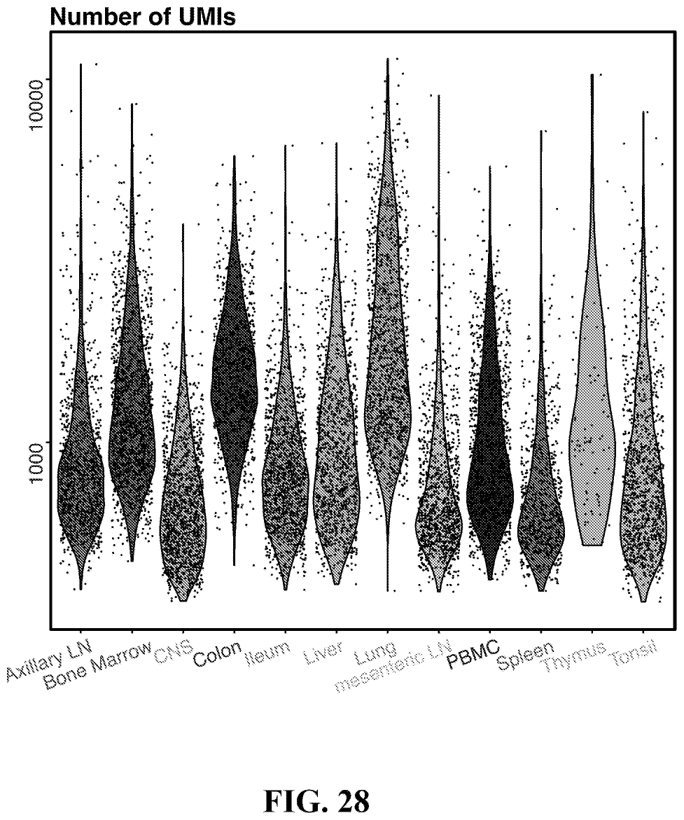

By comparing single cell profiles of healthy subjects with SHIV infected ones, this study identified subsets of cells in specific tissues differentially respond to SHIV infection (FIGS. 25, A and B). In lymphoid tissue, certain immune cells such as CD8 T cells and macrophages appear to be equally represented in both healthy and SHIV infected cells, while other cells such as CD4 T cells and B cells show marked difference between the two states. The comparison further identifies pathways and genes that are differentially expressed in healthy and SHIV infected cells. In CD4 T cells, genes involved in cyclin dependent signaling, chemokine signaling, RNA helicase, mRNA export from nucleus, DNA damage, spliceosome mRNA processing, and transcription regulation are identified as correlated with healthy cells, and genes involved in unfolded protein response, HTLV-1 infection, herpes simplex infection, interferon gamma signaling pathway, antigen processing and presentation via MEW class I, positive regulation of apoptotic process, T cell receptor signaling, virion assembly, and vial transcription are associated with HIV infection (FIGS. 25, C and D). More comprehensively, this study identified gene markers that are differentially expressed in SHIV infected cells. This study also validated the close relationship between SHIV and HIV infection in non-human primate and human cells, by comparing differentially expressed genes between HIV infected and healthy human lymph node cells with SHIV infected and healthy T cells in non-human primates. The significant overlap of the two sets of differentially expressed genes (FIG. 26) confirm that biomarkers identified in this study can further be used in diagnosis, monitoring, and treatment of human HIV related disease.

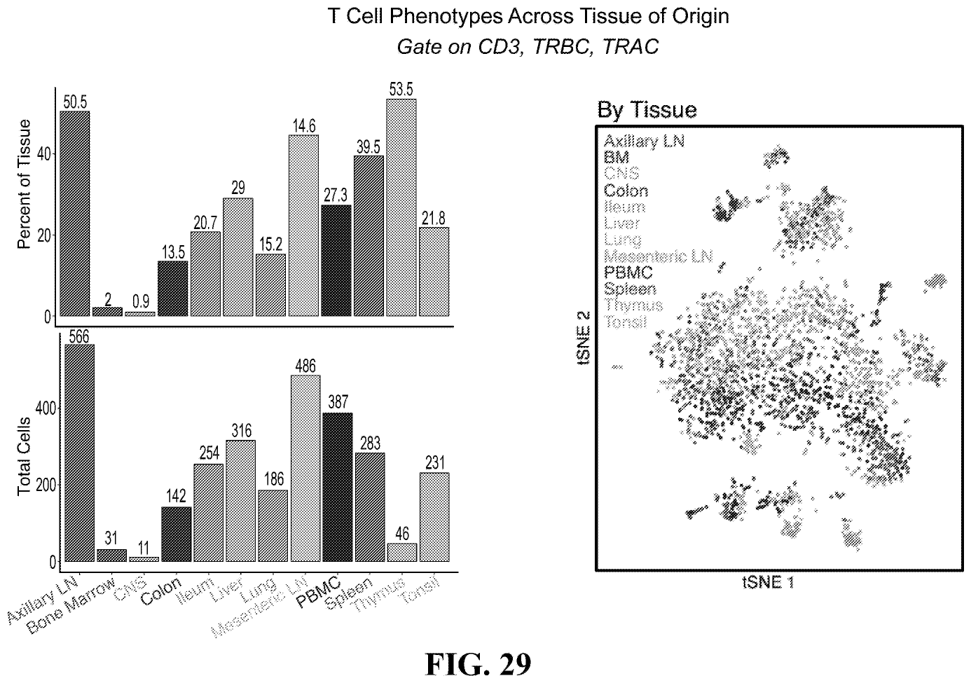

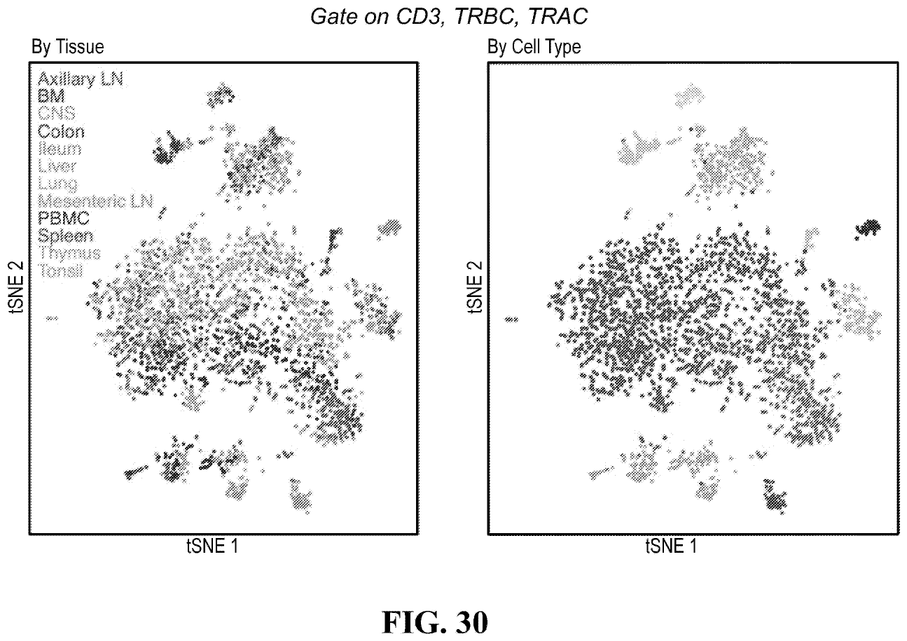

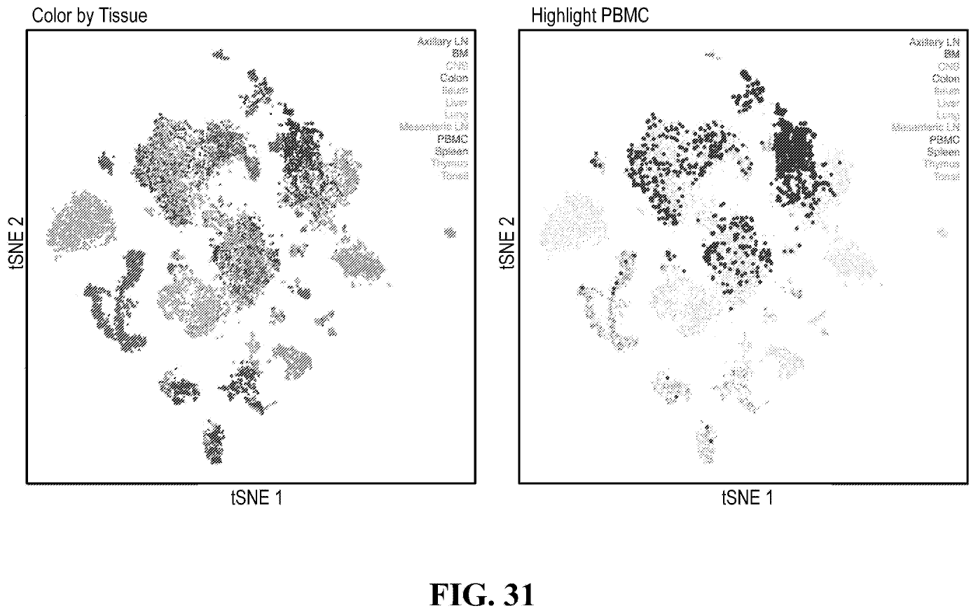

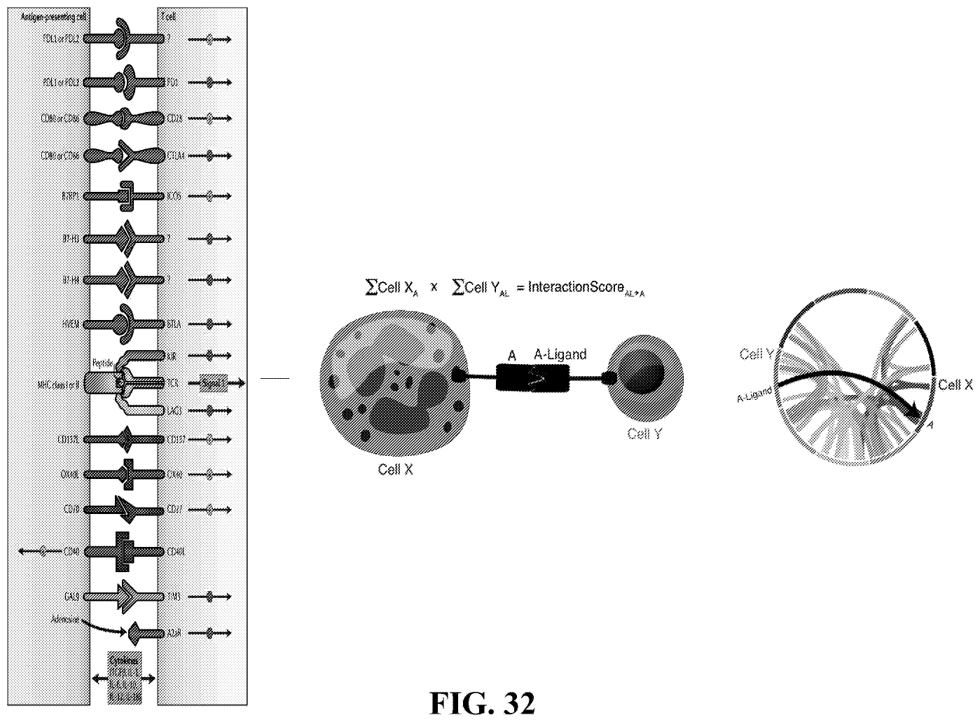

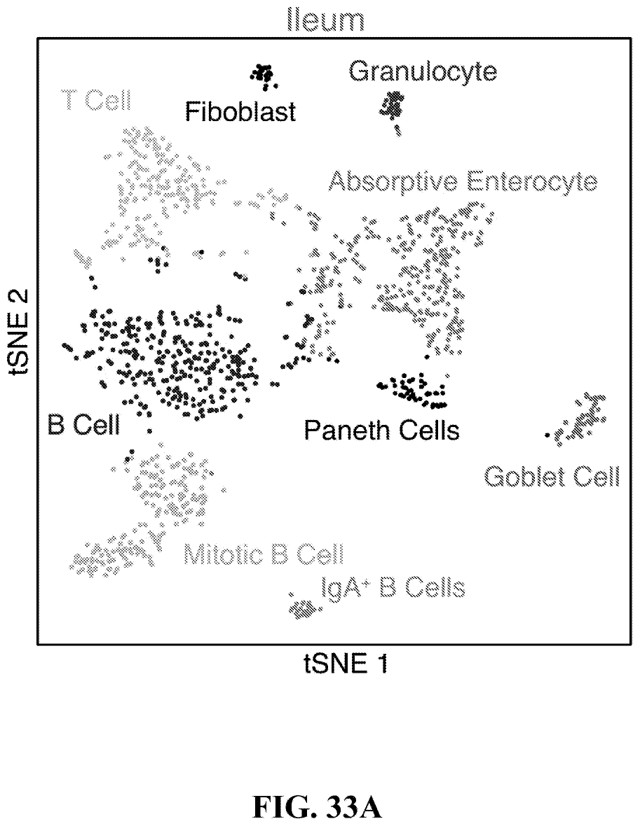

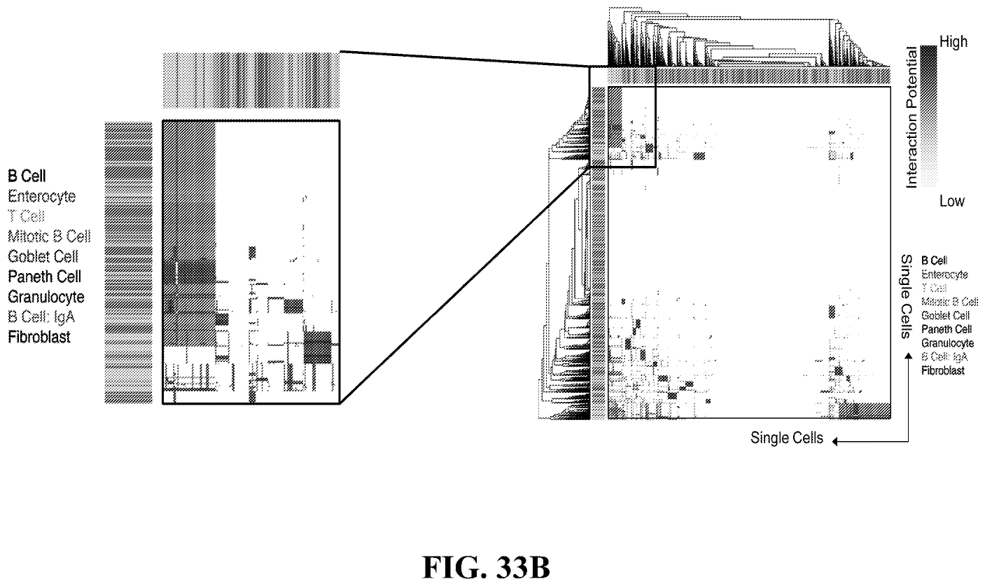

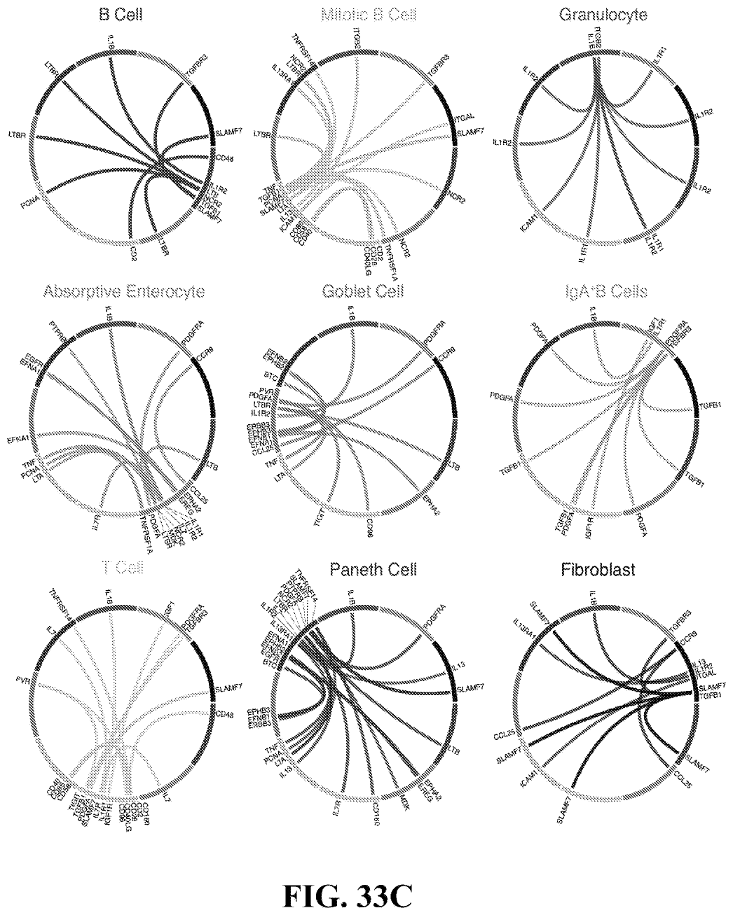

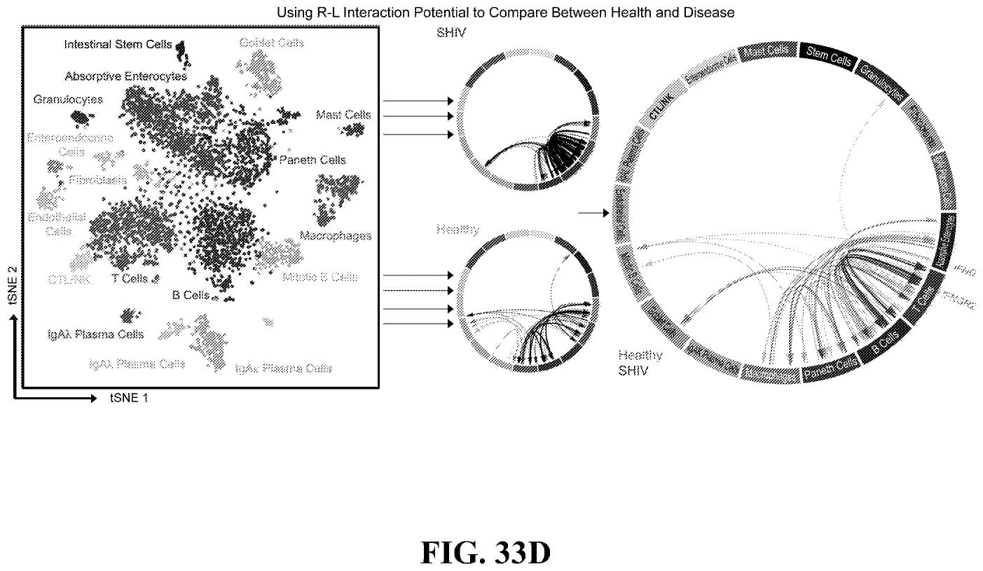

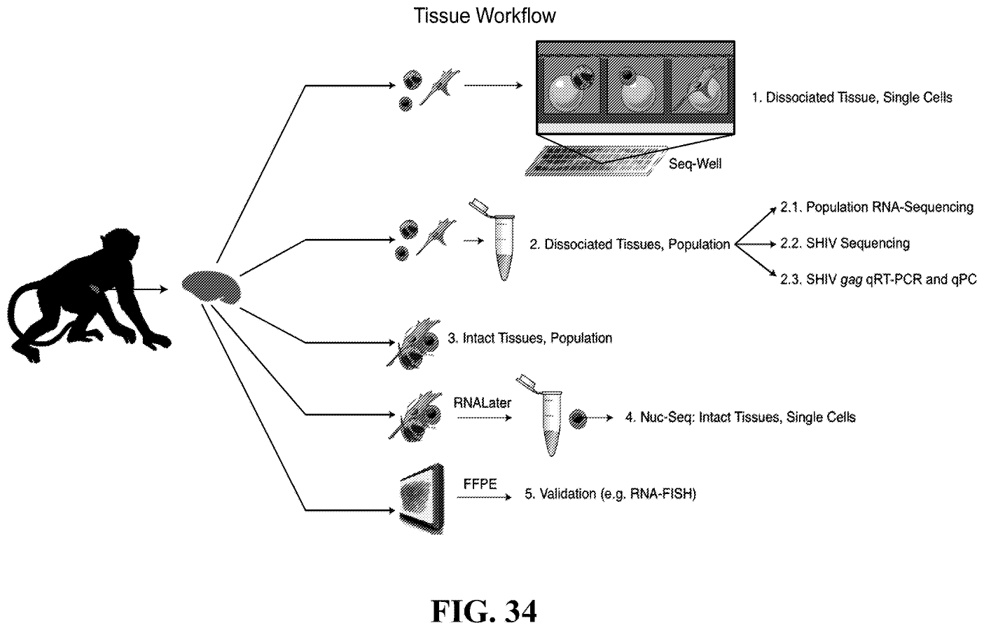

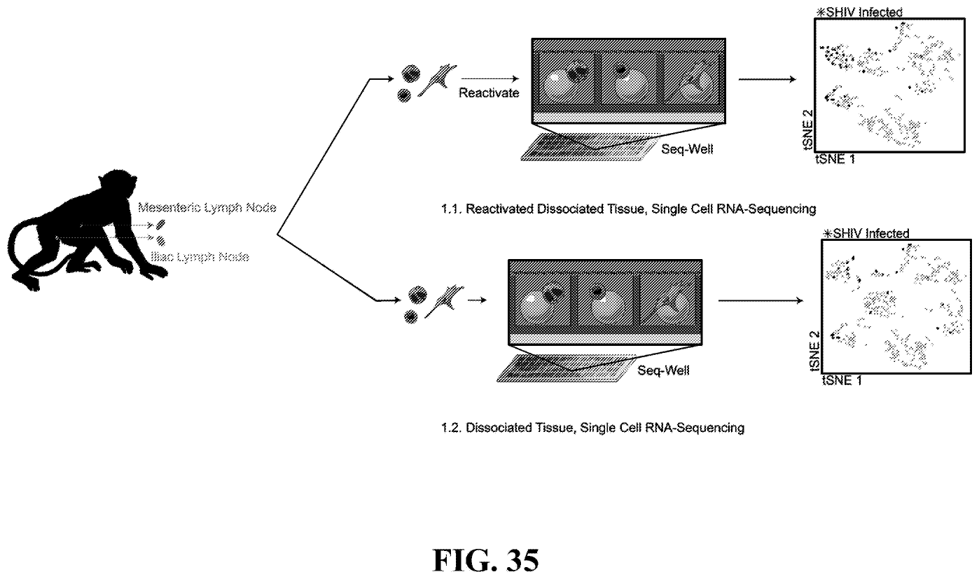

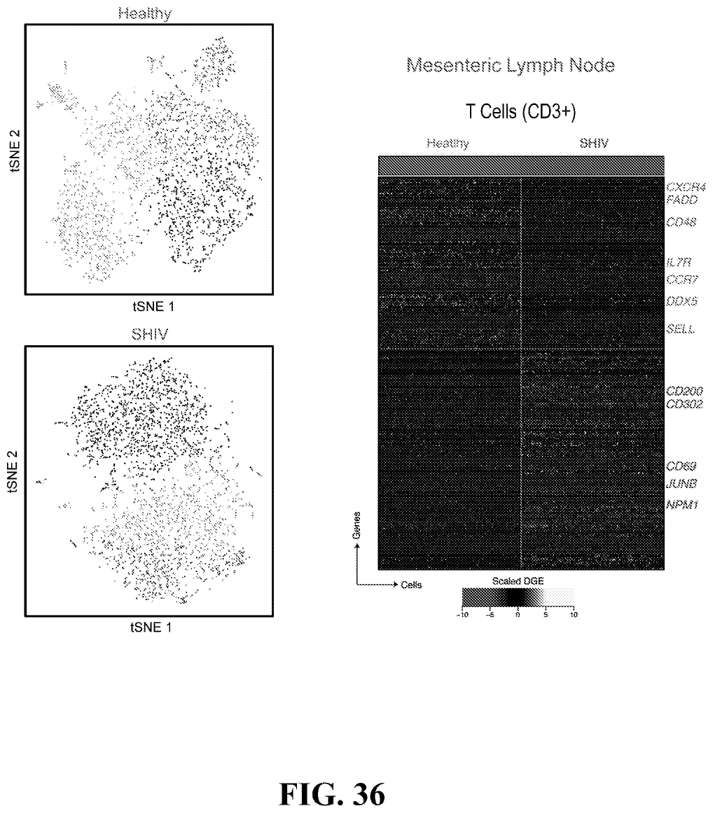

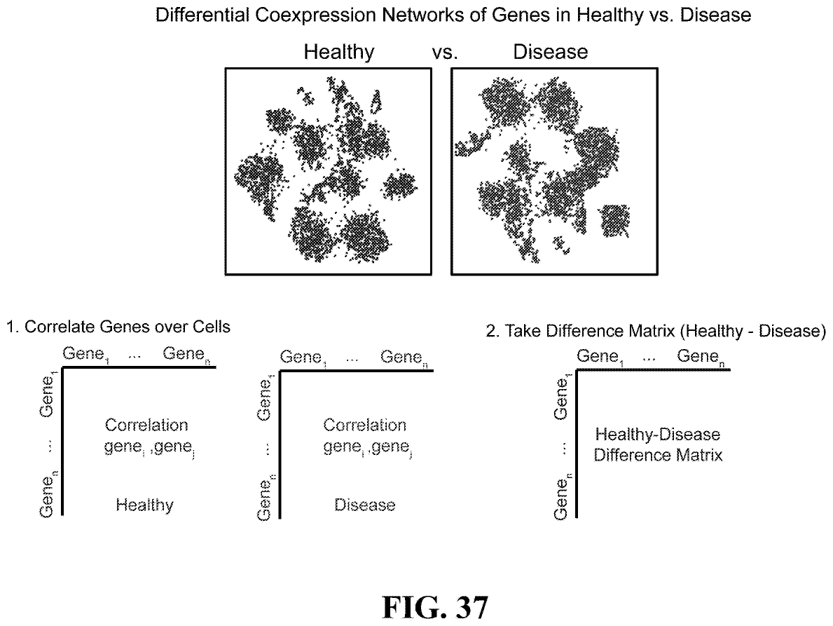

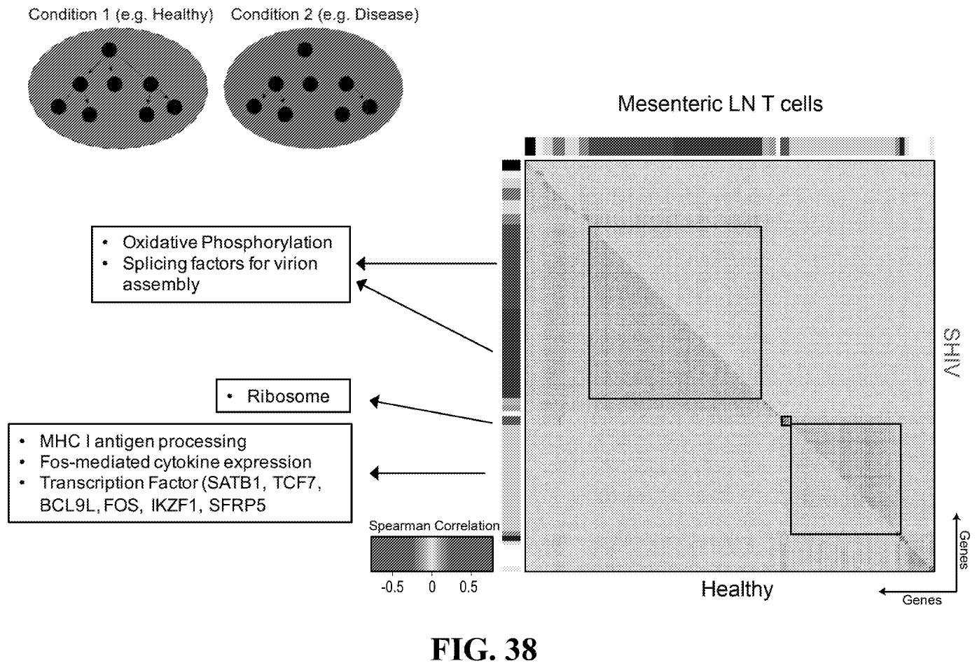

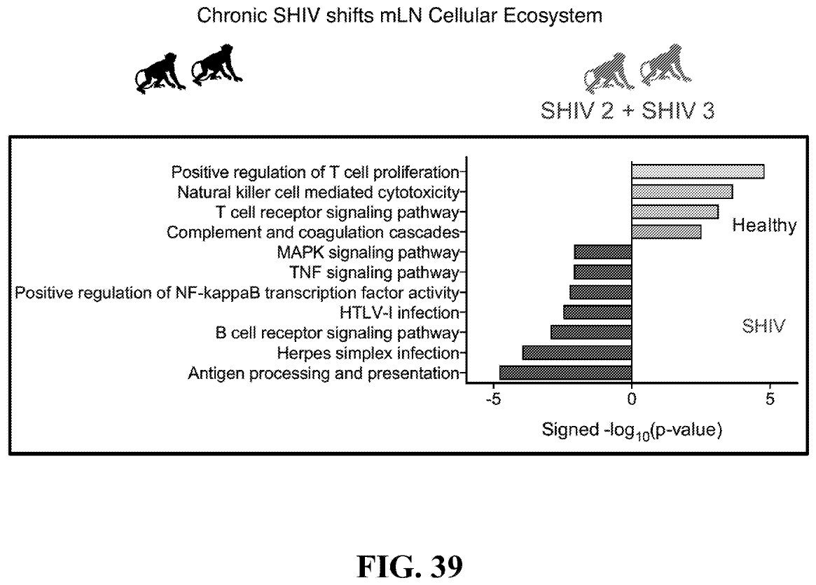

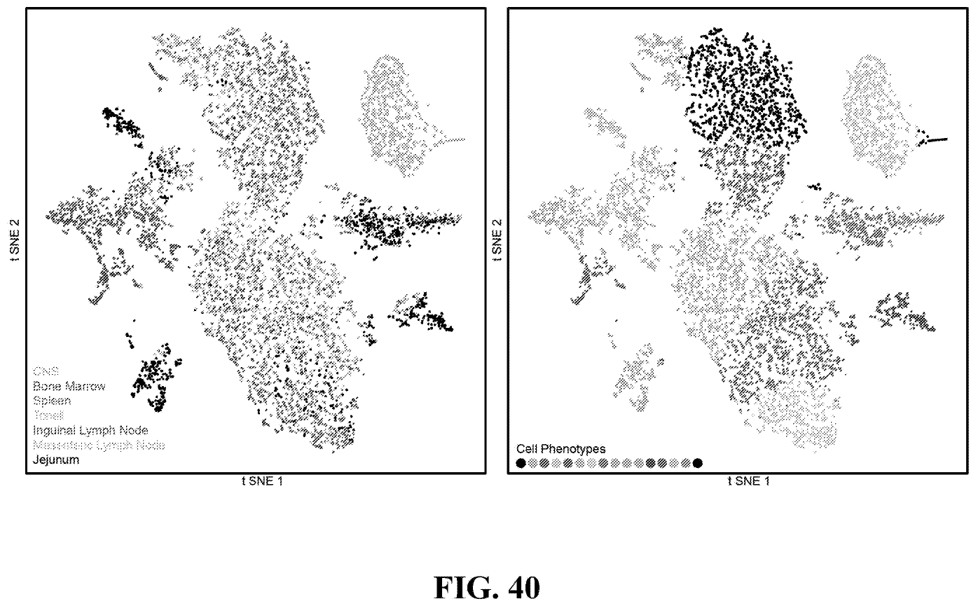

Applicants determined T cell phenotypes across tissue of origins (FIGS. 29 and 30). Applicants observed PBMCs across many tissues and determined gene expression profiles for the PBMCs (FIG. 31). Applicants determined cell-cell interactions based on an interaction score determined by expression of ligand-receptor pairs (FIGS. 32 and 33). Applicants identified genes differentially expressed between healthy and SHIV/ART+ animals (FIGS. 36 and 39). Applicants identified differential coexpression of genes in healthy and SHIV animals (FIG. 38). The diseased samples co-expressed genes related to MEW I antigen processing, Fos-mediated cytokine expression, and transcription factors (SATB1, TCF7, BCL9L, FOS, IKZF1, SFRP5. This cluster of genes was strongly co-expressed in SHIV single T cells and rarely co-expressed in healthy T cells. Applicants constructed a healthy “atlas” of lymphoid tissue.

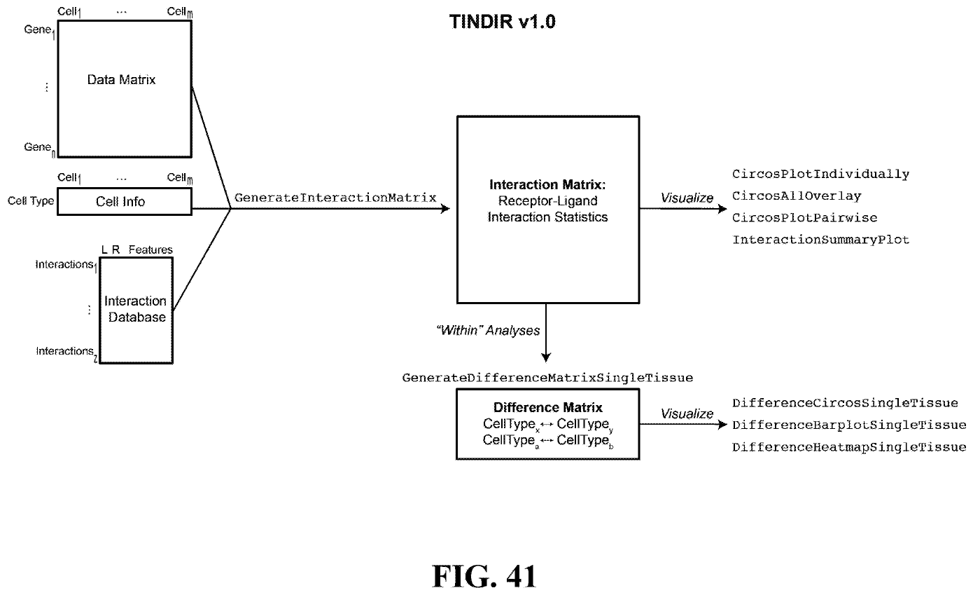

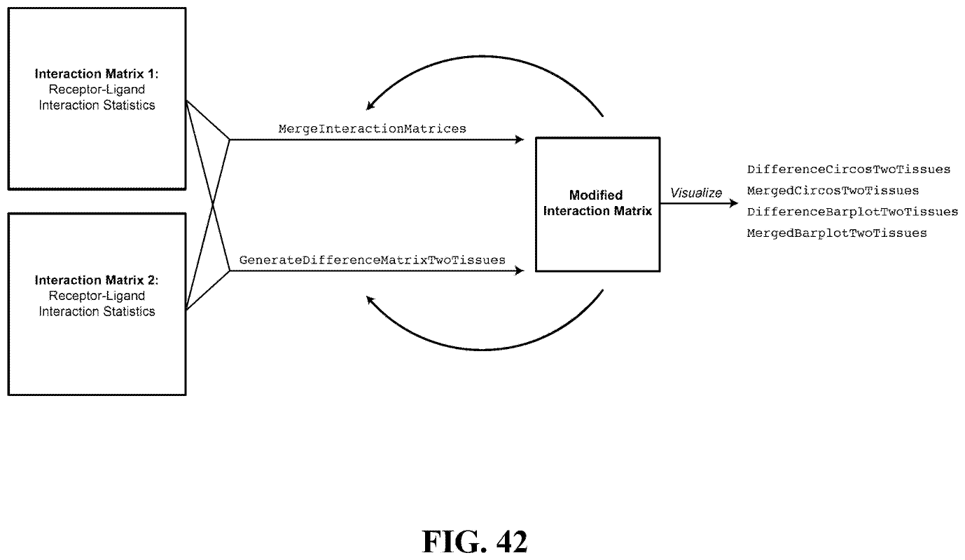

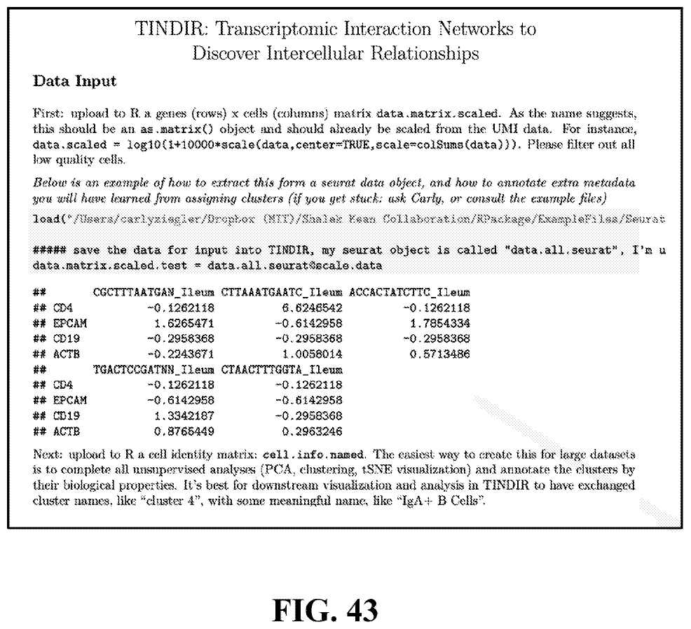

Applicants developed a computer program called Transcriptomic Interaction Networks (TINDIR) to discover intercellular relationships (FIGS. 33, 41-43). TINDIR subsets down the transcriptome to receptor ligand pairs that enable communication. TINDIR is a novel tool to mine single cell data for possible cell-cell interactions, including novel interactions. TINDIR provides for experiential comparison in samples of health and samples of disease. TINDIR can weight these potentials, either on a single cell to single cell level or cell type to cell type level by looking for evidence of a reaction, or a meaningful change in related pathways as a result of both having some receptor, and having nearby cells with ligands to engage. For instance, two cells will have a productive interaction if they have high receptor ligand co-expression. TINDIR can re-classify single cells by their interaction potentials and identify differential properties associated with cell interaction changes (e.g. how do T cells behave when “interacting” with B cells compared to when interacting with Enterocytes)

TINDIR uses an R package, which specifically takes inputs as a data matrix, cell info, and an interaction database (e.g., DIP). TINDIR uses various functions that allow a user to mine for receptor ligand interactions, visualize these, and complete complex analyses on the interaction data

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of determining a physiological state of a first cell or tissue in a subject, the method comprising:
measuring a physiological state of a second cell or tissue in the subject that is correlated with the physiological state of the first cell or tissue,
wherein the correlation comprises a correlation between tissue types, cell types, or tissue types and cell types.

2. The method of claim 1, further comprising contacting the first cell or tissue in the subject with a modulating agent; and
measuring the effect of the modulating agent on a second cell or tissue in the subject, wherein the physiological state of the second cell or tissue is correlated with the effect of the modulating agent on the first cell or tissue,
wherein the correlation comprises a correlation between tissue types, cell types, or tissue types and cell types,
preferably, wherein the modulating agent is an immune modulating agent.

3. The method of claim 1, wherein the composition and/or quantity of cell types in different tissues is correlated, or
wherein the same cell types in different tissues are correlated, or
wherein different cell types are correlated.

4. The method of claim 1, wherein the second cell or tissue is correlated with the first cell or tissue in another organism, whereby the correlation is used as a proxy to determine the physiological state of the first cell or tissue in the subject, preferably,
wherein the organism is a non-human primate, more preferably, wherein the non-human primate is a Rhesus macaque; and/or
wherein the correlation is determined by measuring gene expression profiles in two or more cells or tissues obtained from the organism.

5-7. (canceled)

8. The method of claim 1, wherein the correlated physiological states of the first and second cells or tissues are the same physiological states; or
wherein the correlated physiological states of the first and second cells or tissues are different physiological states; and/or
wherein the physiological state of the second cell or tissue is measured by a gene expression profile comprising one or more genes, preferably, wherein the gene expression profile comprises single cell expression profiles; and/or
wherein the physiological state of the second cell or tissue is measured by a gene expression profile comprising one or more gene clusters, preferably,
wherein the gene clusters comprise one or more principle component genes, or
wherein the one or more gene clusters comprise genes having similar function, or
wherein the one or more gene clusters comprise genes that are co-regulated, or
wherein the genes are co-regulated in the tissue or cell during disease, or
wherein the one or more gene clusters comprise genes of a pathway.

9-17. (canceled)

18. The method of claim 1,
wherein the cell type is an immune cell or the tissue type is an immune tissue type, preferably, wherein the cells comprise T cells from mesenteric lymph node, inguinal lymph node, CNS, jejunun, spleen, tonsil, or bone marrow; or wherein the cells comprise macrophages; or
wherein the cells comprise pneumocytes or NK cells; or
wherein the cells comprise cells of axillary lymph node, colon, ileum, liver, spleen, or thymus; or
wherein the cell or tissue type is a diseased cell or tissue type.

19-24. (canceled)

25. The method of claim 1, wherein the physiological state comprises a disease state or an immunological state; or
wherein the physiologic state indicates resistance or sensitivity to a therapy; or
wherein the second cell is a circulating immune cell and the physiological state is an immune state in a tissue.

26-27. (canceled)

28. A method of identifying a biomarker as a proxy for a physiological state of a cell or tissue, the method comprising determining the expression profile of one or more genes in a test cell or tissue obtained from an organism, and identifying the expression profile in the test cell or tissue as a proxy for the physiological state of a second cell or tissue if the expression profile in the test cell or tissue is correlated with the expression profile in the second cell or tissue obtained from the organism; or
determining an expression profile of one or more genes in a test cell or tissue obtained from an organism that correlates with the expression profile in a second cell or tissue obtained from the organism, preferably,
wherein the expression profile comprises one or more single cell expression profiles and the single cell expression profiles in the test cell or tissue correlates to the single cell expression profiles in the second cell or tissue; and/or
wherein the test cell or tissue is from the same species as the second cell or tissue, more preferably, wherein the test cell or tissue and the second cell or tissue are from a non-human primate, more preferably, wherein the test cell or tissue and the second cell or tissue are from a Rhesus macaque; and/or
wherein the expression profile determined in the test cell or tissue is a proxy for the physiological state of the second cell in a different species, preferably a related species, more preferably wherein the test cell or tissue and the second cell or tissue are from different non-human primates, more preferably, wherein the test cell or tissue is from a human and the second cell or tissue is from a non-human primate.

29-36. (canceled)

37. The method of claim 28, wherein the biomarker identified in the non-human primate is used to determine the physiological state of a second cell or tissue in a human subject by detection or measuring the biomarker in the first cell or tissue in the human subject.

38. The method of claim 28,
wherein the physiological state comprises a disease state or an immunological state; or
wherein the physiologic state indicates resistance or sensitivity to a therapy.

39. (canceled)

40. The method of claim 1, wherein the method is for diagnosing the physiological state of a cell or tissue in a subject, the method comprising measuring the expression of a biomarker in a test cell or tissue of the subject, wherein the biomarker was identified as a proxy for the physiological state of the diagnosed cell or tissue by determining the expression profile of the biomarker in a first cell or tissue, and identifying the expression profile in the first cell or tissue as a proxy for the physiological state of a second cell or tissue if the expression profile in the first cell or tissue is correlated with the expression profile in the second cell or tissue.

41. The method of claim 40, wherein the first cell or tissue is from the same species as the second cell or tissue, preferably, wherein the first cell or tissue and the second cell or tissue are from a non-human primate, more preferably, wherein the first cell or tissue and the second cell or tissue are from a Rhesus macaque.

42-43. (canceled)

44. The method of claim 28, wherein the method is for identifying a biomarker as a proxy for determining the effect of a modulating agent on a cell or tissue in a subject, the method comprising determining an expression profile of one or more genes in a test cell or tissue obtained from an organism treated with the modulating agent that correlates with the expression profile in a second cell or tissue obtained from the treated organism.

45. A method of identifying cell interactions comprising:
providing single cell gene expression profiles obtained from sequencing single cells from one or more tissues from a subject;
determining expression of receptor/ligand pairs on the single cells from the one or more tissues; and
determining cells that express a receptor and cells that express the ligand for the receptor, preferably, wherein cell interactions are determined in a diseased non-human primate.

46. (canceled)

47. A method of identifying biomarkers of tissue homing comprising:
generating single cell expression profiles of PBMC's obtained from two or more tissues of a non-human primate; and
identifying tissue specific markers expressed by the PBMCs, preferably,
using the PBMCs originating from a tissue of interest as a proxy for the physiological state of the tissue of interest.

48. A method of identifying the tissue of origin of macrophages comprising detecting in a population of cells comprising macrophages one or markers selected from one or more groups consisting of:
a. S100A8, HBB, MNP1A, CAMP, LOC710097, gene 24745, gene 18845, LOC703853, LOC706282 and RTD1B;
b. LOC106994075, PLAC8, CLEC9A, GZMB, IRF8, FCER1A, KNG1, IGFBP6, CCDC50 and NCOA7;
c. C1QB, SEPP1, FABP4, C1QC, GPNMB, APOE, ACP5, YMRM176B, ADAMDEC1 and CCDC152; and/or
d. S100A6, FCGR3, VCAN, FGR, LILRB1, FCN1, AHNAK, FN1, C5AR1, TIMP1.

49. (canceled)

50. A method of identifying tissues and cells that are reservoirs for HIV comprising determining expression of SHIV genes in tissues and/or single cells obtained from a non-human primate infected with SHIV and treated with antiretroviral therapy, preferably, wherein SHIV is reactivated in the tissues and/or single cells before determining expression; or
determining expression of HIV genes in tissues and/or single cells obtained from a subject infected with HIV and treated with antiretroviral therapy, preferably,
wherein HIV is reactivated in the tissues and/or single cells before determining expression; and/or
wherein the tissues and/or single cells are obtained from lymph nodes.

51-54. (canceled)

55. The method of claim 18, wherein the diseased cell or tissue type is infected with HIV, preferably, wherein the physiological state comprises an immunological state associated with HIV infection.

56. (canceled)

57. The method of claim 18, wherein the diseased cell or tissue type is infected with MTB, preferably, wherein the physiological state comprises an immunological state associated with MTB infection.

58. (canceled)


SEQUENCE LISTING (18 sequences)

SEQ ID NO: 1 - protein, 7 aa, Simian virus 40
PKKKRKV

SEQ ID NO: 2 - protein, 16 aa, Artificial Sequence
  Synthetic
KRPAATKKAGQAKKKK

SEQ ID NO: 3 - protein, 9 aa, Artificial Sequence
  Synthetic
PAAKRVKLD

SEQ ID NO: 4 - protein, 11 aa, Artificial Sequence
  Synthetic
RQRRNELKRSP

SEQ ID NO: 5 - protein, 38 aa, Artificial Sequence
  Synthetic
NQSSNFGPMKGGNFGGRSSGPYGGGGQYFAKPRNQGGY

SEQ ID NO: 6 - protein, 42 aa, Artificial Sequence
  Synthetic
RMRIZFKNKGKDTAELRRRRVEVSVELRKAKKDEQILKRRNV

SEQ ID NO: 7 - protein, 8 aa, Artificial Sequence
  Synthetic
VSRKRPRP

SEQ ID NO: 8 - protein, 8 aa, Artificial Sequence
  Synthetic
PPKKARED

SEQ ID NO: 9 - protein, 8 aa, Homo sapiens
  MISC_FEATURE (2)..(2): Xaa a position 2 represents pyrrolysine
PXPKKKPL

SEQ ID NO: 10 - protein, 12 aa, Mus sp.
SALIKKKKKMAP

SEQ ID NO: 11 - protein, 5 aa, Influenza virus
DRLRR

SEQ ID NO: 12 - protein, 7 aa, Influenza virus
PKQKKRK

SEQ ID NO: 13 - protein, 10 aa, Hepatitis D virus
RKLKKKIKKL

SEQ ID NO: 14 - protein, 10 aa, Mus sp.
REKKKFLKRR

SEQ ID NO: 15 - protein, 20 aa, Homo sapiens
KRKGDEVDGVDEVAKKKSKK

SEQ ID NO: 16 - protein, 17 aa, Homo sapiens
RKCLQAGMNLEARKTKK

SEQ ID NO: 17 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 18 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS